(19) United States
(12) Patent Application Publication
(10) Pub. No.: US 2024/0425512 A1
(43) Pub. Date: Dec. 26, 2024

(54) NITROGEN-CONTAINING HETEROCYCLIC COMPOUND, PHARMACEUTICAL COMPOSITION THEREOF AND APPLICATION THEREOF

(71) Applicant: 280 Bio, Inc., South San Francisco, CA (US)

(72) Inventors: Zusheng XU, Shanghai (CN); Yangtong LOU, Shanghai (CN); Tiegang XIE, Shanghai (CN); Jian SHEN, Shanghai (CN); Qingrui SUN, Shanghai (CN); Xin JIN, Shanghai (CN); Kun ZENG, Shanghai (CN)

(73) Assignee: 280 Bio, Inc., South San Francisco, CA (US)

(21) Appl. No.: 18/737,785

(22) Filed: Jun. 7, 2024

(30) Foreign Application Priority Data
Jun. 7, 2023 (CN) 2023106668256
Aug. 22, 2023 (CN) 2023110594857
Nov. 21, 2023 (CN) 2023115555083
Feb. 29, 2024 (CN) 202410227922X

(51) Int. Cl. C07D 487/04; A61K 31/519; A61K 31/551; A61K 31/553; A61P 35/00; C07D 471/04; C07D 519/00

(52) U.S. Cl. CPC C07D 487/04; A61K 31/519; A61K 31/551; A61K 31/553; A61P 35/00; C07D 471/04; C07D 519/00

(57) ABSTRACT

Disclosed is a nitrogen-containing heterocyclic compound, pharmaceutical composition thereof and application thereof. The present disclosure provides a nitrogen-containing heterocyclic compound of formula I, a pharmaceutically acceptable salt thereof, a prodrug thereof, a stereoisomer thereof, a tautomer thereof or an isotopically labeled compound thereof. The nitrogen-containing heterocyclic compounds as represented in Formula I in the present disclosure have good inhibitory effects on cells of various mutations such as KRAS G12C, KRAS G12D, KRAS G12V, KRAS Q61H and other mutations, as well as on KRAS-amplified wild-type cells, and are expected to treat and/or prevent a wide range of diseases associated with Ras.

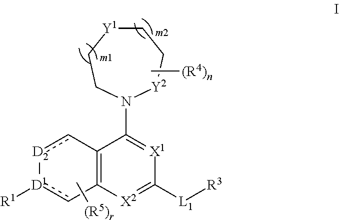

### CROSS-REFERENCE TO RELATED APPLICATIONS

This application claims priority under 35 U.S.C. § 119(b) to Chinese Application No. 2023106668256, filed Jun. 7, 2023, Chinese Application No. 2023110594857, filed Aug. 22, 2023, Chinese Application No. 2023115555083, filed Nov. 21, 2023, and Chinese Application No. 202410227922X, filed Feb. 29, 2024, the disclosures of which are incorporated herein by reference in their entirety.

### TECHNICAL FIELD

The present disclosure relates to a nitrogen-containing heterocyclic compound, pharmaceutical composition thereof and application thereof.

### BACKGROUND

Ras (Rat sarcoma viral oncogene) was first found in rat sarcoma. There are three members in the ras gene family of mammals, namely H-ras (HRAS), K-ras (KRAS) and N-ras (NRAS), where the fourth exon of K-ras has variants A and B. Ras gene is widely found in various eukaryotes such as mammals, fruit flies, fungi, nematodes and yeasts, and is expressed at various levels in different tissues, where H-Ras is mainly expressed in skin and skeletal muscle, K-Ras is mainly expressed in colon and thymus, and N-Ras is expressed at a high level in testis. Ras protein regulates and controls signal transduction by switching the binding to GTP/GDP as a molecular switch in cell signal transduction, thereby regulating the life processes such as proliferation, differentiation, senescence and apoptosis of cells.

The mutated forms of RAS are closely associated with the occurrence and development of human tumors, and are present in about 30% of human tumors. KRAS mutation is most common and accounts for approximately 85% of cases, and NRAS and HRAS account for 12% and 3%, respectively. KRAS mutation is mainly found in pancreatic, colorectal and lung cancers, NRAS mutation is common in melanoma and acute myelogenous leukemia, and HRAS mutation is common in bladder and head and neck cancers. Ras proto-oncogene mutation occurs mainly by point mutation. More than 150 different Ras point mutations have been found, with mutations in the glycine at positions 12 and 13 and glutamine at position 61 being the most common.

For decades, efforts have been made to develop small molecule inhibitors targeting Ras. Scientists have been hoping to develop competitive inhibitors of GTP that act directly on the Ras protein. However, this has not been successful because of the strong affinity between GTP and Ras (pmol/L level), the high concentration of GTP in cells (0.5 mM), the lack of a pocket in the RAS protein structure that facilitates the binding of small molecules, and the like. In recent years, some advances have been made in drug development using the allosteric site of KRasG^{G12C} mutant. In 2013, a team of researchers reported the discovery about K-Ras^{G12C} small molecule inhibitors (Nature, 2013, 503, 548-551). They identified a novel binding pocket located below the molecular switch II region from K-Ras G12C mutant. Those inhibitors bind to the allosteric pocket and covalently bind to nearby Cys12, thereby selectively inhibiting the activation of K-Ras G12C. Other researchers have reported KRAS inhibitors with cellular activity (Science, 2016, 351, 604-608).

As one of the most important cancer driver genes, the mutation of KRAS G12C provides the possibility of irreversible covalent small molecule drug design, and the research and development of the corresponding drugs have become the hotspot of the global anti-tumor drug research and development after the breakthrough of related scientific research work. In May 2021, Amgen's KRAS G12C inhibitor Lumakras received accelerated approval from the U.S. FDA, achieving a major breakthrough in the “non-druggable” nature of KRAS.

Despite the major breakthrough in KRAS G12C inhibitor research, current clinical studies of KRAS G12C inhibitors have shown that they are not effective for long periods of time and are prone to drug resistance. Meanwhile, numerous other mutations, including cancers harboring KRAS G12D, G12V, G13D, G12R, G12S, G12A and other mutations, as well as KRAS wild-type amplification, lack effective drugs. Therefore, there is an urgent need to find drugs that target KRAS mutations.

In conclusion, after decades of unremitting efforts, the understanding of Ras has been gradually improved, but only one drug for KRAS G12C mutation has been marketed so far, and there is no particularly effective treatment for other different mutations. The search for compounds with good inhibitory effects on Ras remains a hot and difficult research area in new drug development.

### Content of the Present Invention

The technical problem to be solved by the present disclosure is the lack of effective drugs serving as Ras inhibitors for clinical treatment in the prior art. Therefore, the present disclosure provides a nitrogen-containing heterocyclic compound, pharmaceutical composition thereof and application thereof, and the nitrogen-containing heterocyclic compound is expected to treat or prevent various RAS-related diseases.

The present disclosure solves the above problem by the following technical schemes.

The present disclosure provides a nitrogen-containing heterocyclic compound of formula I, a pharmaceutically acceptable salt thereof, a prodrug thereof, a stereoisomer thereof, a tautomer thereof or an isotopically labeled compound thereof:

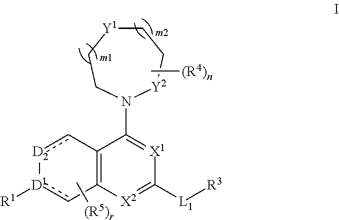

wherein [symbol] represents a single or double bond;

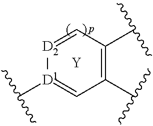

is phenyl, “5- to 7-membered heterocycloalkenyl containing 1-3 heteroatoms independently selected from O, S and N”, “5- to 7-membered heteroaryl containing 1-3 heteroatoms independently selected from O, S and N” or 5- to 7-membered cycloalkenyl; wherein D¹ is C, CH or N; D₂ is

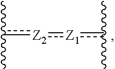

wherein Z₁ and Z₂ are independently a bond, CH, CH₂, O, S, N or NH;
r is 0, 1, 2, 3 or 4;
R⁵ is independently deuterium, halogen, C₁₋₆ alkyl, C₁₋₆ alkyl substituted with one or more halogen, C₃₋₁₀ cycloalkyl, hydroxyl, C₁₋₆ alkyl-O—, C₁₋₆ alkyl-S—, amino, C₁₋₆ alkyl-NH—, C₂₋₆ alkenyl, nitro or C₃₋₁₀ cycloalkyl-O—;
X¹ and X² are independently CR^{b} or N, and X¹ and X² are not both CR^{b};
Y¹ is O, S, NH, CH₂ or

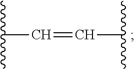 [SMILES: *C=C*]

Y² is NR², O or S;
m1, m2 and n are independently 0, 1, 2, 3 or 4;
R^{b} is hydrogen, halogen or cyano;
R¹ is C₆₋₂₀ aryl, “5- to 12-membered heteroaryl containing 1-4 heteroatoms independently selected from O, S and N”, C₆₋₂₀ aryl substituted with one or more R¹⁻¹ or “5- to 12-membered heteroaryl containing 1-4 heteroatoms independently selected from O, S and N” substituted with one or more R¹⁻²; provided that when multiple substituents are present, the substituents are the same or different;
R¹⁻¹ and R¹⁻² are independently deuterium, halogen, —OR^{c}, cyano, azide, —C(═O)R¹¹, —NR¹²R¹³, —C(═O)OR¹⁴, —C(═O)NR¹⁵R¹⁶, C₁₋₆ alkyl, C₂₋₆ alkenyl, C₂₋₆ alkynyl, C₃₋₁₀ cycloalkyl, —B(OR^{x}) (OR^{y}), “4- to 7-membered heterocycloalkyl containing 1 or 2 heteroatoms independently selected from O, S and N”, C₆₋₂₀ aryl, “5- to 7-membered heteroaryl containing 1 or 2 heteroatoms independently selected from O, S, and N”, C₁₋₆ alkyl substituted by one or more R¹⁻¹⁻¹, C₁₋₆ alkyl-O— substituted by one or more R¹⁻¹⁻², C₃₋₁₀ cycloalkyl substituted by one or more R¹⁻¹⁻³, “4- to 7-membered heterocyclic cycloalkyl containing 1 or 2 heteroatoms independently selected from O, S, and N” substituted by one or more R¹⁻¹⁻⁴, C₆₋₂₀ aryl substituted with one or more R¹⁻¹⁻⁵, or “5- to 7-membered heteroaryl containing one or two heteroatoms independently selected from O, S, and N” substituted with one or more R¹⁻¹⁻⁶; provided that when multiple substituents are present, the substituents are the same or different; or, when the number of R¹⁻¹ or R¹⁻² is more than one, two optional R¹⁻¹ or R¹⁻² are connected, together with the atoms on the ring to which they are attached, independently form 3- to 8-membered cyclic olefin;
R², R^{c}, R¹² and R¹³ are independently hydrogen, C₁₋₆ alkyl, —C(═O)R^{c1}, —C(═O)OR^{c2}, —C(═O)NR^{c3}R^{c4}, —SO₂R^{c5},

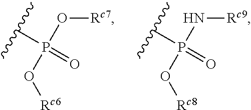

or C₁₋₆ alkyl substituted with one or more R^{c10}; R^{c1}, R^{c2}, R^{c3}, R^{c4}, R^{c5}, R^{c6}, R^{c7}, R^{c8} and R^{c9} are independently hydrogen, C₁₋₂₀ alkyl, C₃₋₁₀ cycloalkyl, “4- to 7-membered heterocycloalkyl containing 1 or 2 heteroatoms independently selected from O, S and N”, C₆₋₂₀ aryl, “5- to 7-membered heteroaryl containing 1 or 2 heteroatoms independently selected from O, S and N”, C₁₋₂₀ alkyl substituted with one or more R⁴⁻¹⁻¹, C₃₋₁₀ cycloalkyl substituted with one or more R⁴⁻¹⁻², “4- to 7-membered heterocycloalkyl containing 1 or 2 heteroatoms independently selected from O, S and N” substituted with one or more R⁴⁻¹⁻³, C₆₋₂₀ aryl substituted with one or more R⁴⁻¹⁻⁴, “5- to 7-membered heteroaryl containing 1 or 2 heteroatoms independently selected from O, S and N”, “5- to 7-membered heteroaryl containing 1 or 2 heteroatoms independently selected from O, S and N” substituted with one or more R⁴⁻¹⁻⁵, —NR^{c51}R^{c52}, —(C₁₋₆ alkylene-O)ₚ— C₁₋₆ alkyl,

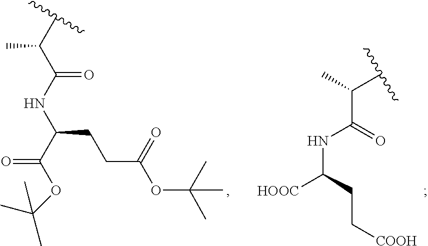 [SMILES: *[C@@H](C)C(=O)N[C@@H](CCC(=O)OC(C)(C)C)C(=O)OC(C)(C)C ; *[C@@H](C)C(=O)N[C@@H](CCC(=O)O)C(=O)O]

p is 1, 2,3 or 4; R¹⁻¹⁻¹, R¹⁻¹⁻², R¹⁻¹⁻³, R¹⁻¹⁻⁴, R¹⁻¹⁻⁵, R¹⁻¹⁻⁶, R⁴⁻¹⁻², R⁴⁻¹⁻³, R⁴⁻¹⁻⁴ and R⁴⁻¹⁻⁵ are independently cyano, halogen, hydroxyl, nitro, C₁₋₆ alkyl-O—, C₁₋₆ alkyl, C₁₋₆ alkyl substituted with one or more R³¹, C₃₋₁₀ cycloalkyl, “4- to 7-membered heterocycloalkyl containing 1 or 2 heteroatoms independently selected from O, S, and N”, C₆₋₂₀ aryl, “5- to 7-membered heteroaryl containing 1 or 2 heteroatoms independently selected from O, S, and N”, —B(OR^{x}) (OR^{y}), —C(═O)R²¹, —NR²²R²³, —C(═O)OR²⁴, OC(═O)R³², —C(═O)NR²⁵R²⁶, —COOH, —C(═O)OR²⁷ or —OC(═O)OR²⁸; R^{c10} is —OC₁₋₆ alkyl, hydroxyl,

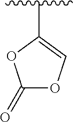 [SMILES: *c1coc(=O)o1]

substituted with C₁₋₆ alkyl, C₆₋₂₀ aryl substituted with one or more —B(OR^{x})(OR^{y}), “5- to 7-membered heteroaryl containing 1 or 2 heteroatoms independently selected from O, S, and N” substituted with one or more R¹⁰⁻¹,

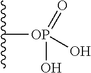

or —OC(═O)R²⁹; R¹⁰⁻¹ is independently C₁₋₆ alkyl or nitro;
or, optional R² and R^{b}, optional R² and R⁵ together with the atoms to which they are attached, independently form “5- to 12-membered heterocyclic alkenyl containing 2 to 4 heteroatoms, two of which are N, and the other heteroatoms independently selected from O, S, and N”, or “5- to 12-membered heterocyclic alkenyl containing 2 to 4 heteroatoms, two of which are N, and the other heteroatoms independently selected from O, S, and N,” substituted with one or more R^{a};
R^{x} and R^{y} are independently hydrogen, C₁₋₆ alkyl, or R^{x} and R^{y} each with the attached oxygen atom form five-membered ring substituted with one or more C₁₋₆ alkyl;
R⁴ is independently deuterium, halogen, cyano, hydroxyl, C₁₋₆ alkyl, C₁₋₆ alkyl substituted with one or more R⁴⁻¹, C₂₋₆ alkenyl, C₂₋₆ alkynyl, C₃₋₁₀ cycloalkyl, C₁₋₆ alkyl-O—, —O—C₁₋₆ alkyl substituted with one or more R⁴⁻², —S—C₁₋₆ alkyl, —S—C₁₋₆ alkyl substituted with one or more R⁴⁻³, O═,

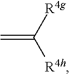

O—C₃₋₁₀ cycloalkyl, —C(═O)R^{4a}, —NR^{4b1}R^{4b2}, —C(═O)OR^{4c} or —C(═O)NR^{4d1}R^{4d2}; or, when n is 2, 3 or 4, two optional R⁴ are connected, together with the atoms to which they are attached form 3- to 8-membered carbocyclic ring, 3- to 8-membered carbocyclic ring substituted with one or more R⁴⁻⁴, “4- to 8-membered heterocyclic ring containing 1 to 3 heteroatoms independently selected from O, S, and N”, or, “4- to 8-membered heterocyclic ring containing 1 to 3 heteroatoms independently selected from O, S, and N” substituted with one or more R⁴⁻⁵;
R⁴⁻¹, R⁴⁻², R⁴⁻³, R⁴⁻⁴, R⁴⁻⁵ and R^{a} are independently halogen, C₁₋₆ alkyl, C₂₋₆ alkenyl, cyano, hydroxyl, C₁₋₆ alkyl-O—, —NR⁴ⁱR^{4j}, —C(═O)OR^{4e} or —C(═O)NR^{4f1}R^{4f2}; or, R⁴⁻⁵ is O═;
R^{4a}, R^{4b1}, R^{4b2}, R^{4c}, R^{4d1}, R^{4d2}, R^{4e}, R^{4f1}, R^{4f2}, R⁴ⁱ and R^{4j} are independently hydrogen, C₁₋₆ alkyl or C₂₋₆ alkenyl;
L₁ is a bond, C₁₋₆ alkylene, —C(═O)—, —O(R^{L-1})ₙ₁—, —S(R^{L-2})ₙ₂— or —NR^{L-3}(R^{L-4})ₙ₃—; R^{L-1}, R^{L-2} and R^{L-4} are independently C₁₋₆ alkylene or C₁₋₆ alkylene substituted with one or more deuterium; R^{L-3} is hydrogen or C₁₋₆ alkyl; n1, n2 and n3 are independently 0 or 1;
R³ is C₃₋₁₂ cycloalkyl, C₃₋₁₂ cycloalkyl substituted with one or more R³⁻¹, “4- to 12-membered heterocycloalkyl containing 1-3 heteroatoms independently selected from O, S and N”, “4- to 12-membered heterocycloalkyl containing 1-3 heteroatoms independently selected from O, S and N” substituted with one or more R³⁻², C₁₋₆ alkyl, C₁₋₆ alkyl substituted with one or more R³⁻³, C₂₋₆ alkenyl substituted with one or more R³⁻⁴, —OR^{d}, —SR^{d1}, —NR^{e1}R^{e2} or —C(═O)NR^{e3}R^{e4}; provided that when multiple substituents are present, the substituents are the same or different;
R³⁻¹, R³⁻², R³⁻³ and R³⁻⁴ are independently C₁₋₆ alkyl, C₁₋₆ alkyl substituted with one or more R³⁻¹⁻¹ C₆₋₂₀ aryl substituted with one or more R³⁻¹⁻⁵, hydroxyl, azide, Ar, C₁₋₆ alkyl-O—, —CH₂—O—Ar, halogen, O═, —NR^{e5}R^{e6}, —C(═O)NR^{e7}R^{e8} or

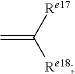

Ar is “5-12-membered heteroaryl containing 1-3 heteroatoms independently selected from O, N and S” substituted with one or more R³⁻¹⁻⁴; or, R³⁻¹ and R³⁻² are independently -L₂-M; L₂ is -L₂₁-L₂₂-L₂₃-L₂₄-L₂₅-L₂₆; L₂₁, L₂₃ and L₂₅ are independently a bond, O, S, NH, C(O)NH or C₁₋₆ alkylene; L₂₂ and L₂₄ are independently a bond, C₃₋₁₀ cycloalkyl, “4- to 7-membered heterocycloalkyl containing 1 or 2 heteroatoms independently selected from O, S and N”, C₆₋₂₀ aryl, or, “5- to 7-membered heteroaryl containing 1 or 2 heteroatoms independently selected from O, S, and N”; L₂₆ is a bond, O, S, NH or C(O)NH;
M is

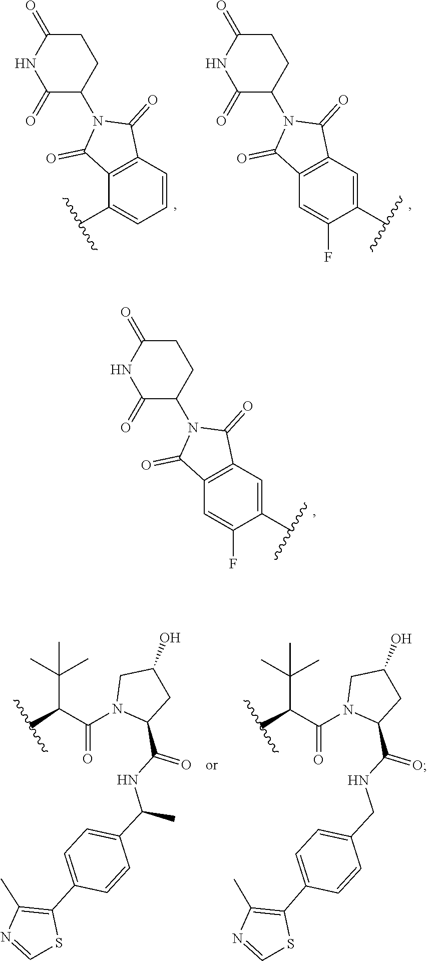 [SMILES: *c1cccc2c1C(=O)N(C1CCC(=O)NC1=O)C2=O ; *c1cc2c(cc1F)C(=O)N(C1CCC(=O)NC1=O)C2=O ; *c1cc2c(cc1F)C(=O)N(C1CCC(=O)NC1=O)C2=O ; *[C@H](C(=O)N1C[C@H](O)C[C@H]1C(=O)N[C@@H](C)c1ccc(-c2scnc2C)cc1)C(C)(C)C ; *[C@H](C(=O)N1C[C@H](O)C[C@H]1C(=O)NCc1ccc(-c2scnc2C)cc1)C(C)(C)C]

R^{4g}R^{4h}, R^{e17} and R^{e18} are independently hydrogen, halogen, C₁₋₆ alkyl, C₁₋₆ alkyl substituted with one or more halogen, cyano, —C(═O)R^{e171}, —C(═O)OR^{e172} or —C(═O)NR^{e173}R^{e174}; or, R^{4g} and R^{4h}, R^{e17} and R^{e18}, together with the atom to which they are attached form 3- to 8-membered carbocyclic ring, or “4- to 8-membered heterocyclic ring containing 1 to 3 heteroatoms independently selected from O, S, and N”;
R^{d}, R^{d1}, R^{e1}, R^{e2}, R^{e3} and R^{e4} are independently hydrogen, C₁₋₆ alkyl, C₃₋₁₀ cycloalkyl, “4- to 10-membered heterocycloalkyl containing 1-3 heteroatoms independently selected from O, S and N”, or, C₁₋₆ alkyl substituted with one or more R³⁻¹⁻²;
R³⁻¹⁻¹, R³⁻¹⁻², R³⁻¹⁻⁵ and R³⁻¹⁻⁴ are independently deuterium, cyano, halogen, hydroxyl, C₁₋₆ alkyl, C₂₋₆ alkenyl, C₁₋₆ alkyl-O—, C₁₋₆ alkyl-O— substituted with one or more halogen, C₁₋₆ alkyl substituted with one or more halogen, —C(═O)R^{e9}, —NR^{e10}R^{e11}, —C(═O)OR^{e12}, —C(═O)NR^{e13}R^{e14}, —OC(═O)NR^{e15}R^{e16}, “4- to 10-membered heterocycloalkyl containing 1-3 heteroatoms independently selected from O, S and N”, or, “4- to 10-membered heterocycloalkyl containing 1-3 heteroatoms independently selected from O, S and N” substituted with one or more R³⁻¹⁻¹⁻¹; or, two optional R³⁻¹⁻⁵ are connected, together with the atoms to which they are attached form “4- to 10-membered heterocycloalkyl containing 1-3 heteroatoms independently selected from O, S and N” substituted with one or more R³⁻¹⁻¹⁻²;
R³⁻¹⁻¹⁻¹ and R³⁻¹⁻¹⁻² are independently

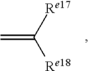

halogen, C₁₋₆ alkyl or C₆₋₂₀ aryl substituted with one or more R³⁻¹⁻¹⁻¹⁻¹; R³⁻¹⁻¹⁻¹⁻¹ is halogen or C₁₋₆ alkyl substituted with one or more halogen; R^{e5}, R^{e6}, R^{e7}, R^{e8}, R^{e9}, R^{e10}, R^{e11}, R^{e12}, R^{e13}, R^{e14}, R^{e15}, R^{e16}, R^{e171}, R^{e172}, R^{e173} and R^{e174} are independently hydrogen or C₁₋₆ alkyl;
or, R¹² and R¹³, R¹⁵ and R¹⁶, R^{c3} and R^{c4}, R²² and R²³, R²⁵ and R²⁶, R^{4b1} and R^{4b2}, R^{4d1} and R^{4d2}, R^{4f1} and R^{4f2}, R⁴ⁱ and R^{4j}, R^{e1} and R^{e2}, R^{e3} and R^{e4}, R^{e5} and R^{e6}, R^{e7} and R^{e8}, R^{e10} and R^{e11}, R^{e13} and R^{e14}, R^{e15} and R^{e16}, R^{e173} and R^{e174} together with the atom N to which they are attached form “4- to 12-membered heterocycloalkyl containing 1-3 heteroatoms, of which one is N, the other independently selected from O, S and N”, or, “4- to 12-membered heterocycloalkyl containing 1-3 heteroatoms, of which one is N, the other independently selected from O, S and N” substituted with one or more R³⁻¹⁻³; R³⁻¹⁻³ is independently deuterium, cyano, halogen, hydroxyl, C₁₋₆ alkyl-O—, C₁₋₆ alkyl, C₁₋₆ alkyl substituted with one or more R³³, “4- to 7-membered heterocycloalkyl containing 1 or 2 heteroatoms independently selected from O, S and N” or —C(═O)OR³⁰.
R^{c51}, R^{c52}, R¹¹, R²¹, R¹⁴, R¹⁵, R¹⁶, R²⁴, R²⁵, R²⁶, R²⁷, R²⁸ and R³⁰ are independently hydrogen or C₁₋₆ alkyl;
R²⁹ is hydrogen, C₁₋₆ alkyl or —NR^{m}Rⁿ;
R³¹ is hydrogen, C₁₋₆ alkyl, —OC₁₋₆ alkyl, —NR^{m}Rⁿ, —OC(═O)R³² or —C(═O)OC₁₋₆ alkyl;
R³² and R³³ are independently —OC₁₋₆ alkyl or C₁₋₆ alkyl substituted with one or more —NR^{m}Rⁿ; R²², R²³, R^{m} and Rⁿ are independently hydrogen, C₁₋₆ alkyl or Boc.

In a certain embodiment, the nitrogen-containing heterocyclic compound of formula I, the pharmaceutically acceptable salt thereof, a prodrug thereof, a stereoisomer thereof, a tautomer thereof or an isotopically labeled compound thereof as described above, wherein, [symbol] represents a single or double bond;

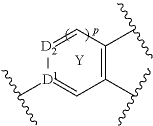

is phenyl, “5- to 7-membered heterocycloalkenyl containing 1-3 heteroatoms independently selected from O, S and N” or “5- to 7-membered heteroaryl containing 1-3 heteroatoms independently selected from O, S and N”; wherein D¹ is C, CH or N; D₂ is

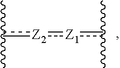

(wherein Z₁ and Z₂ are independently a bond, CH, CH₂, O, S, N or NH;
r is 0, 1 or 2;
R⁵ is independently halogen, C₁₋₆ alkyl, C₁₋₆ alkyl substituted with one or more halogen, hydroxyl, C₁₋₆ alkyl-O—, C₂₋₆ alkenyl or nitro;
Y¹ is O, NH, CH₂ or;

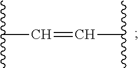 [SMILES: *C=C*]

Y² is NR² or 0;
m1, m2 and n are independently 0, 1, 2, 3 or 4;
X¹ and X² are N;
R¹ is C₆₋₂₀ aryl, “5- to 12-membered heteroaryl containing 1-4 heteroatoms independently selected from O, S and N”, C₆₋₂₀ aryl substituted with one or more R¹⁻¹ or “5- to 12-membered heteroaryl containing 1-4 heteroatoms independently selected from O, S and N” substituted with one or more R¹⁻²; provided that when multiple substituents are present, the substituents are the same or different;
R¹⁻¹ and R¹⁻² are independently halogen, —OR^{c}, cyano, —NR¹²R¹³, C₁₋₆ alkyl, C₂₋₆ alkenyl, C₂₋₆ alkynyl, —B(OR^{x}) (OR^{y}) or C₁₋₆ alkyl substituted by one or more R¹⁻¹⁻¹; provided that when multiple substituents are present, the substituents are the same or different;
R², R^{c}, R¹² and R¹³ are independently hydrogen, C₁₋₆ alkyl, —C(═O)R^{c}, —C(═O)OR^{c2}, -C(═O)NR^{c3}R^{c4}, —SO₂R^{c5},

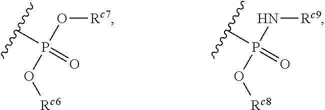

or C₁₋₆ alkyl substituted with one or more R^{c10} R^{c1}, R^{c2}, R^{c3}, R^{c4}, R^{c5}, R^{c6}, R^{c7}, R^{c8} and R^{c9} are independently hydrogen, C₁₋₂₀ alkyl, C₃₋₁₀ cycloalkyl, C₁₋₂₀ alkyl substituted with one or more R⁴⁻¹⁻¹, C₆₋₂₀ aryl substituted with one or more R⁴⁻¹⁻⁴, “5- to 7-membered heteroaryl containing 1 or 2 heteroatoms independently selected from O, S and N”, “5- to 7-membered heteroaryl containing 1 or 2 heteroatoms independently selected from O, S and N” substituted with one or more R⁴⁻¹⁻⁵, —NR^{c51}R^{c52}, —(C₁₋₆ alkylene-O)ₚ—C₁₋₆ alkyl,

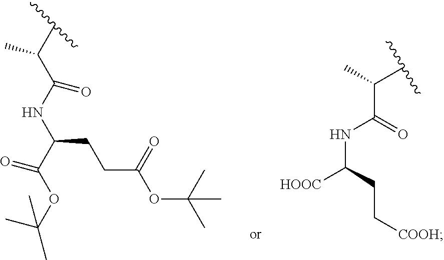 [SMILES: *[C@@H](C)C(=O)N[C@@H](CCC(=O)OC(C)(C)C)C(=O)OC(C)(C)C ; *[C@@H](C)C(=O)N[C@@H](CCC(=O)O)C(=O)O]

p is 1, 2, 3 or 4; provided that when multiple substituents are present, the substituents are the same or different;
R¹⁻¹⁻¹, R⁴⁻¹⁻¹, R⁴⁻¹⁻⁴ and R⁴⁻¹⁻⁵ are independently halogen, hydroxyl, C₁₋₆ alkyl-O—, C₁₋₆ alkyl substituted with one or more R³¹, “4- to 7-membered heterocycloalkyl containing 1 or 2 heteroatoms independently selected from O, S, and N”, C₆₋₂₀ aryl, —C(═O)R²¹, —NR²²R²³, —C(═O)OR²⁴, OC(═O)R³², —COOH, —C(═O)OR²⁷ or —OC(═O)OR²⁸;
R^{c10} is —OC₁₋₆ alkyl, hydroxyl,

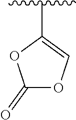 [SMILES: *c1coc(=O)o1]

substituted with C₁₋₆ alkyl, C₆₋₂₀ aryl substituted with one or more —B(OR^{x})(OR^{y}), “5- to 7-membered heteroaryl containing 1 or 2 heteroatoms independently selected from O, S, and N” substituted with one or more R^{c10-1},

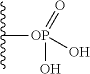

or —OC(═O)R²⁹; R^{c10-1} is independently C₁₋₆ alkyl or nitro;
or, optional R² and R⁵ together with the atoms to which they are attached, independently form “5- to 12-membered heterocyclic alkenyl containing 2 to 4 heteroatoms, two of which are N, and the other heteroatoms independently selected from O, S, and N”;
R^{x} and R^{y} are independently hydrogen or C₁₋₆ alkyl, or R^{x} and R^{y} each with the attached oxygen atom form five-membered ring substituted with one or more C₁₋₆ alkyl;
R⁴ is independently deuterium, halogen, hydroxyl, C₁₋₆ alkyl, O═ or —C(═O)R^{4a}; or, when n is 2, 3 or 4, two optional R⁴ are connected, together with the atoms to which they are attached form “4- to 8-membered heterocyclic ring containing 1 to 3 heteroatoms independently selected from O, S, and N” substituted with one or more R⁴⁻⁵;
R⁴⁻⁵ is O═;
R^{4a} is C₁₋₆ alkyl or C₂₋₆ alkenyl;
L₁ is a bond or —O(R^{L-1})ₙ₁—; R^{L-1} is C₁₋₆ alkylene or C₁₋₆ alkylene substituted with one or more deuterium; n1 is 1;
R³ is C₃₋₁₂ cycloalkyl substituted with one or more R³⁻¹, “4- to 12-membered heterocycloalkyl containing 1-3 heteroatoms independently selected from O, S and N”, “4- to 12-membered heterocycloalkyl containing 1-3 heteroatoms independently selected from O, S and N” substituted with one or more R³⁻² or C₂₋₆ alkenyl substituted with one or more R³⁻⁴; provided that when multiple substituents are present, the substituents are the same or different;
R³⁻¹, R³⁻² and R³⁻⁴ are independently C₁₋₆ alkyl, C₁₋₆ alkyl substituted with one or more R³⁻¹⁻¹, C₆₋₂₀ aryl substituted with one or more R³⁻¹⁻⁵, Ar, C₁₋₆ alkyl-O—, —CH₂—O—Ar, halogen or

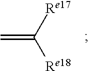

Ar is “5-12-membered heteroaryl containing 1-3 heteroatoms independently selected from O, N and S” substituted with one or more R³⁻¹⁻⁴;
R³⁻¹⁻¹, R³⁻¹⁻⁵ and R³⁻¹⁻⁴ are independently halogen, C₁₋₆ alkyl, C₂₋₆ alkenyl, C₁₋₆ alkyl-O— substituted with one or more halogen, C₁₋₆ alkyl substituted with one or more halogen, —C(═O)R^{e9}, —NR^{e10}R^{e11}, —OC(═O)NR^{e15}R^{e16}, “4- to 10-membered heterocycloalkyl containing 1-3 heteroatoms independently selected from O, S and N”, or, “4- to 10-membered heterocycloalkyl containing 1-3 heteroatoms independently selected from O, S and N” substituted with one or more R³⁻¹⁻¹⁻¹; or, two optional R³⁻¹⁻⁵ are connected, together with the atoms to which they are attached form “4- to 10-membered heterocycloalkyl containing 1-3 heteroatoms independently selected from O, S and N” substituted with one or more R³⁻¹⁻¹⁻²;
R³⁻¹⁻¹⁻¹ and R³⁻¹⁻¹⁻² are independently

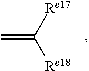

halogen, C₁₋₆ alkyl or C₆₋₂₀ aryl substituted with one or more R³⁻¹⁻¹⁻¹⁻¹; R³⁻¹⁻¹⁻¹⁻¹ is independently halogen or C₁₋₆ alkyl substituted with one or more halogen;
or, R³⁻¹ and R³⁻² are independently -L₂-M; L₂ is -L₂₁-L₂₂-L₂₃-L₂₄-L₂₅-L₂₆; L₂₁, L₂₃ and L₂₅ are independently a bond, O, S, NH, C(O)NH or C₁₋₆ alkylene; L₂₂ and L₂₄ are independently a bond, C₃₋₁₀ cycloalkyl, “4- to 7-membered heterocycloalkyl containing 1 or 2 heteroatoms independently selected from O, S and N”, C₆₋₂₀ aryl, or, “5- to 7-membered heteroaryl containing 1 or 2 heteroatoms independently selected from O, S, and N”; L₂₆ is a bond, O, S, NH or C(O)NH;
M is

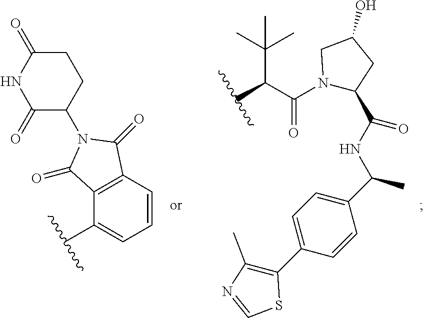 [SMILES: *c1cccc2c1C(=O)N(C1CCC(=O)NC1=O)C2=O ; *[C@H](C(=O)N1C[C@H](O)C[C@H]1C(=O)N[C@@H](C)c1ccc(-c2scnc2C)cc1)C(C)(C)C]

R^{e17} and R^{e18} are independentlyhydrogen or halogen;
R^{e9}, R^{e10}, R^{e11}, R^{e15} and R^{e16} are independently hydrogen or C₁₋₆ alkyl;
or, R^{c3} and R^{c4} together with the atom N to which they are attached form “4- to 12-membered heterocycloalkyl containing 1-3 heteroatoms, of which one is N, the other independently selected from O, S and N”, or, “4- to 12-membered heterocycloalkyl containing 1-3 heteroatoms, of which one is N, the other independently selected from O, S and N” substituted with one or more R³⁻¹⁻³; R³⁻¹⁻³ is independently C₁₋₆ alkyl substituted with one or more R³³, “4- to 7-membered heterocycloalkyl containing 1 or 2 heteroatoms independently selected from O, S and N” or —C(═O)OR³⁰.

R^{c51}, R^{c52}, R²⁴, R²⁷, R²⁸ and R³⁰ are independently hydrogen or C₁₋₆ alkyl;
R²⁹ is hydrogen, C₁₋₆ alkyl or —NR^{m}Rⁿ;
R³¹ is hydrogen, C₁₋₆ alkyl, —OC₁₋₆ alkyl, —NR^{m}Rⁿ, —OC(═O)R³² or —C(═O)OC₁₋₆ alkyl;
R³² and R³³ are independently-OC₁₋₆ alkyl or C₁₋₆ alkyl substituted with one or more —NR^{m}Rⁿ; R²¹, R²², R²³, R^{m} and Rⁿ are independentlyhydrogen, C₁₋₆ alkyl or Boc.

In a certain embodiment, the nitrogen-containing heterocyclic compound of formula I, the pharmaceutically acceptable salt thereof, a prodrug thereof, a stereoisomer thereof, a tautomer thereof or an isotopically labeled compound thereof as described above, wherein, “ [symbol] ” represents a single or double bond;

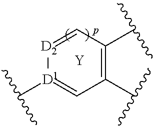

is phenyl, “5- to 7-membered heterocycloalkenyl containing 1-3 heteroatoms independently selected from O, S and N” or “5- to 7-membered heteroaryl containing 1-3 heteroatoms independently selected from O, S and N”; wherein D¹ is C or N; D₂ is

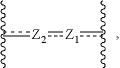

wherein Z₁ and Z₂ are independently a bond, CH, CH₂N or NH;
r is 0, 1 or 2;
R⁵ is independently halogen, C₁₋₆ alkyl, C₁₋₆ alkyl substituted with one or more halogen, hydroxyl or C₂₋₆ alkenyl;
Y¹ is O, NH, CH₂ or

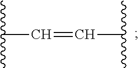 [SMILES: *C=C*]

Y² is NR² or O; R² is hydrogen, C₁₋₆ alkyl, —C(═O)R^{c1} or —C(═O)OR^{c2}; R^{c1} is C₁₋₂₀ alkyl substituted with one or more R⁴⁻¹⁻¹; R^{c2} is C₁₋₆ alkyl; R⁴⁻¹⁻¹ is independently halogen;
m1 and m2 are independently 0 or 1;
n is 0, 1 or 2;
R⁴ is halogen, hydroxyl or C₁₋₆ alkyl;
X¹ and X² are N;
R¹ is C₆₋₂₀ aryl, “5- to 12-membered heteroaryl containing 1-4 heteroatoms independently selected from O, S and N”, C₆₋₂₀ aryl substituted with one or more R¹⁻¹ or “5- to 12-membered heteroaryl containing 1-4 heteroatoms independently selected from O, S and N” substituted with one or more R¹⁻²; provided that when multiple substituents are present, the substituents are the same or different;
R¹⁻¹ and R¹⁻² are independently halogen, —OR^{c}, cyano, —NR¹²R¹³, C₁₋₆ alkyl, C₂₋₆ alkenyl, C₂₋₆ alkynyl, —B(OR^{x}) (OR^{y}) or C₁₋₆ alkyl substituted by one or more R¹⁻¹⁻¹; provided that when multiple substituents are present, the substituents are the same or different;
R^{c} is hydrogen, —C(═O)R^{c1}, —C(═O)NR^{c3}R^{c4} or C₁₋₆ alkyl substituted with one or more R^{c10}; R^{c10} is independently —OC(═O)R²⁹; R²⁹ is —NR^{m}Rⁿ; R^{m} and Rⁿ are C₁₋₆ alkyl;
R^{c1}, R^{c3} and R^{c4} are independently hydrogen, C₁₋₂₀ alkyl, C₃₋₁₀ cycloalkyl, —(C₁₋₆ alkylene-O)ₚ—C₁₋₆ alkyl, C₁₋₂₀ alkyl substituted with one or more R⁴⁻¹⁻¹ or C₆₋₂₀ aryl substituted with one or more R⁴⁻¹⁻⁴; p is 1,2 or 3;
R⁴⁻¹⁻¹ is independently C₁₋₆ alkyl-O—, —C(═O)R²¹, —NR²²R²³, —COOH or “4- to 7-membered heterocycloalkyl containing 1 or 2 heteroatoms independently selected from O, S, and N”; R²¹ is C₁₋₆ alkyl; R²² and R²³ are independently hydrogen, C₁₋₆ alkyl or Boc;
R⁴⁻¹⁻⁴ is halogen;
or, R^{c3} and R^{c4} together with the atom N to which they are attached form “4- to 12-membered heterocycloalkyl containing 1-3 heteroatoms, of which one is N, the other independently selected from O, S and N”, or, “4- to 12-membered heterocycloalkyl containing 1-3 heteroatoms, of which one is N, the other independently selected from O, S and N” substituted with one or more R³⁻¹⁻³; R³⁻¹⁻³ is independently C₁₋₆ alkyl substituted with one or more R³³ or “4- to 7- membered heterocycloalkyl containing 1 or 2 heteroatoms independently selected from O, S and N”; R³³ is independently —OC₁₋₆ alkyl;
R¹² and R¹³ are independently hydrogen;
R^{x} and R^{y} are independently hydrogen;
R¹⁻¹⁻¹ is halogen;
L₁ is a bond or —O(R^{L-1})ₙ₁—; R^{L-1} is C₁₋₆ alkylene or C₁₋₆ alkylene substituted with one or more deuterium; n1 is 1;
R³ is C₃₋₁₂ cycloalkyl substituted with one or more R³⁻¹, “4- to 12-membered heterocycloalkyl containing 1-3 heteroatoms independently selected from O, S and N”, “4- to 12-membered heterocycloalkyl containing 1-3 heteroatoms independently selected from O, S and N” substituted with one or more R³⁻² or C₂₋₆ alkenyl substituted with one or more R³⁻⁴; provided that when multiple substituents are present, the substituents are the same or different;
R³⁻¹ is independently C₁₋₆ alkyl substituted with one or more R³⁻¹⁻¹; R³⁻¹⁻¹ is independently —NR^{e10}R^{e11}, “4- to 10-membered heterocycloalkyl containing 1-3 heteroatoms independently selected from O, S and N”, or, “4- to 10-membered heterocycloalkyl containing 1-3 heteroatoms independently selected from O, S and N” substituted with one or more R³⁻¹⁻¹⁻¹; R^{e10} and R^{e11} are independently C₁₋₆ alkyl; R³⁻¹⁻¹⁻¹ is independently

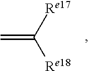

C₁₋₆ alkyl or C₆₋₂₀ aryl substituted with one or more R³⁻¹⁻¹⁻¹⁻¹; R³⁻¹⁻¹⁻¹⁻¹ is independently halogen;
R³⁻² is independently halogen,

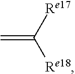

C₁₋₆ alkyl, C₁₋₆ alkyl substituted with one or more R³⁻¹⁻¹ Ar, —CH₂—O—Ar or C₆₋₂₀ aryl substituted with one or more R³⁻¹⁻⁵; Ar is “5-12-membered heteroaryl containing 1-3 heteroatoms independently selected from O, N and S” substituted with one or more R³⁻¹⁻⁴; R³⁻¹⁻¹ is halogen, C₂₋₆ alkenyl or —OC(═O)NR^{e15}R^{e16}; R^{e15} and R^{e16} are independently C₁₋₆ alkyl; R³⁻¹⁻⁵ and R³⁻¹⁻⁴ are independently C₁₋₆ alkyl, halogen, C₁₋₆ alkyl-O— substituted with one or more halogen or C₁₋₆ alkyl substituted with one or more halogen; or, two optional R³⁻¹⁻⁵ are connected, together with the atoms to which they are attached form “4- to 10-membered heterocycloalkyl containing 1-3 heteroatoms independently selected from O, S and N” substituted with one or more R³⁻¹⁻¹⁻²; R³⁻¹⁻¹⁻² is independently halogen;
or, R³⁻¹ is -L₂-M; L₂ is -L₂₁-L₂₂-L₂₃-L₂₄-L₂₅-L₂₆; L₂₁, L₂₃ and L₂₅ are independently a bond, O, S, NH, C(O)NH or C₁₋₆ alkylene; L₂₂ and L₂₄ are independently a bond, C₃₋₁₀ cycloalkyl, “4- to 7-membered heterocycloalkyl containing 1 or 2 heteroatoms independently selected from O, S and N”, C₆₋₂₀ aryl, or, “5- to 7-membered heteroaryl containing 1 or 2 heteroatoms independently selected from O, S, and N”; L₂₆ is a bond, O, S, NH or C(O)NH;
M is

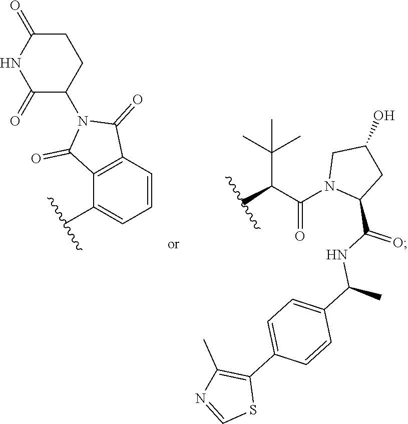 [SMILES: *c1cccc2c1C(=O)N(C1CCC(=O)NC1=O)C2=O ; *[C@H](C(=O)N1C[C@H](O)C[C@H]1C(=O)N[C@@H](C)c1ccc(-c2scnc2C)cc1)C(C)(C)C]

R³⁻⁴ is independently halogen or C₁₋₆ alkyl substituted with one or more R³⁻¹⁻¹; R³⁻¹⁻¹ is independently —NR^{e10}R^{e11} or “4- to 10-membered heterocycloalkyl containing 1-3 heteroatoms independently selected from O, S and N”; R^{e10} and R^{e11} are independently C₁₋₆ alkyl;
R^{e17} and R^{e18} are independently hydrogen or halogen.

In a certain embodiment, with regard to a nitrogen-containing heterocyclic compound of formula I, a pharmaceutically acceptable salt thereof, a prodrug thereof, a stereoisomer thereof, a tautomer thereof or an isotopically labeled compound thereof, some groups are as defined as follows, and the unmentioned group definitions are described in any one of the embodiments of the present disclosure (this content is hereinafter referred to simply as “in a certain embodiment”).

In a certain embodiment, two R⁴ on the same carbon atom form

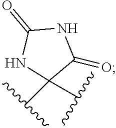 [SMILES: *C1(*)NC(=O)NC1=O]

or, two R⁴ optional form 3- to 8-membered carbonyl ring.

In a certain embodiment, D¹ is C or N.

In a certain embodiment, in D₂, one of Z₁ and Z₂ is CH, CH₂ or N; the other one is a bond.

In a certain embodiment, r is 0, 1 or 2.

In a certain embodiment, R⁵ is independently halogen, C₁₋₆ alkyl, C₁₋₆ alkyl substituted with one or more halogen, hydroxyl, C₁₋₆ alkyl-O—, C₂₋₆ alkenyl or nitro.

In a certain embodiment, when r is 1,

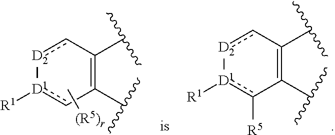

In a certain embodiment, X¹ and X² are N.

In a certain embodiment, R¹⁻¹ is independently halogen, —OR^{c}, C₁₋₆ alkyl, —NR¹²R¹³, C₂₋₆ alkynyl or C₁₋₆ alkyl substituted by one or more R¹⁻¹⁻¹; provided that when multiple substituents are present, the substituents are the same or different; R¹⁻¹⁻¹ is independently halogen.

In a certain embodiment, R¹⁻² is independently halogen, cyano, C₂₋₆ alkenyl or —NR¹²R¹³.

In a certain embodiment, R¹² and R¹³ are independently hydrogen, —SO₂R^{c5} or —C(═O)OR^{c2}; R^{c2} is C₁₋₆ alkyl.

In a certain embodiment, R^{c} is hydrogen, —C(═O)R^{c1}, —C(═O)OR^{c2}, —C(═O)NR^{c3}R^{c4}, —SO₂R^{c5},

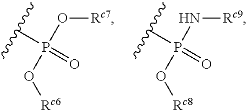

or C₁₋₆ alkyl substituted with one or more R^{c10}; R^{c10} is independently —OC₁₋₆ alkyl,

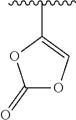 [SMILES: *c1coc(=O)o1]

substituted with C₁₋₆ alkyl, C₆₋₂₀ aryl substituted with one or more —B(OR^{x})(OR^{y}), “5- to 7-membered heteroaryl containing 1 or 2 heteroatoms independently selected
from O, S, and N” substituted with one or more R^{c10-1},

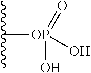

or —OC(═O)R²⁹R¹ independently C₁₋₆ alkyl or nitro; R^{c1}, R^{c2}, R^{c3}, R^{c4}, R^{c5}, R^{c6}, R^{c7}, R^{d8} and R^{d9} are independently hydrogen, C₁₋₂₀ alkyl, C₃₋₁₀ cycloalkyl, C₁₋₂₀ alkyl substituted with one or more R⁴⁻¹⁻¹, C₆₋₂₀ aryl substituted with one or more R⁴⁻¹⁻⁴, “5- to 7-membered heteroaryl containing 1 or 2 heteroatoms independently selected from O, S and N”, “5- to 7-membered heteroaryl containing 1 or 2 heteroatoms independently selected from O, S and N” substituted with one or more R⁴⁻¹⁻⁵, -NR^{c51}R^{c52}, —(C₁₋₆ alkylene-O)ₚ—C₁₋₆ alkyl,

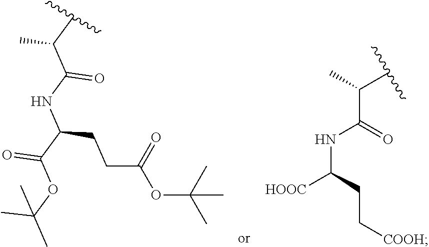 [SMILES: *[C@@H](C)C(=O)N[C@@H](CCC(=O)OC(C)(C)C)C(=O)OC(C)(C)C ; *[C@@H](C)C(=O)N[C@@H](CCC(=O)O)C(=O)O]

p is 1, 2,3 or 4;
provided that when multiple substituents are present, the substituents are the same or different; R¹⁻¹⁻¹R⁴⁻¹⁻¹, R⁴⁻¹⁻⁴ and R⁴⁻¹⁻⁵ are independently halogen, hydroxyl, C₁₋₆ alkyl-O—, C₁₋₆ alkyl, C₁₋₆ alkyl substituted with one or more R³¹, “4- to 7-membered heterocycloalkyl containing 1 or 2 heteroatoms independently selected from O, S, and N”, C₆₋₂₀ aryl, “5- to 7-membered heteroaryl containing 1 or 2 heteroatoms independently selected from O, S and N”, —NR²²R²³, —C(═O)OR²⁴, —OC(═O)R³², —COOH, —C(═O)OR²⁷ or —OC(═O)OR²⁸; R^{x} and R^{y} are independently hydrogen, or R^{x} and R^{y} each with the attached oxygen atom form five-membered ring substituted with one or more C₁₋₆ alkyl.

In a certain embodiment, when p is more than 1, each C₁₋₆ alkylene is the same or different.

In a certain embodiment, R^{c3} and R^{c4} together with the atom N to which they are attached form “4- to 12-membered heterocycloalkyl containing 1-3 heteroatoms, of which one is N, the other independently selected from O, S and N”, or, “4- to 12-membered heterocycloalkyl containing 1-3 heteroatoms, of which one is N, the other independently selected from O, S and N” substituted with one or more R³⁻¹⁻³; R³⁻¹⁻³ is independently C₁₋₆ alkyl substituted with one or more R³³, “4- to 7-membered heterocycloalkyl containing 1 or 2 heteroatoms independently selected from O, S and N” or —C(═O)OR³⁰; R³⁰ is hydrogen or C₁₋₆ alkyl; R³³ is independently-OC₁₋₆ alkyl or C₁₋₆ alkyl substituted with one or more —NR^{m}Rⁿ; R^{m} and Rⁿ are independently hydrogen, C₁₋₆ alkyl or Boc.

In a certain embodiment, when R¹ is C₆₋₂₀ aryl substituted with one or more R¹⁻¹, the more is two or three.

In a certain embodiment, Y¹ is O, NH, CH₂ or

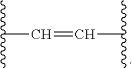 [SMILES: *C=C*]

In a certain embodiment, m1, m2 and n are independently 0, 1 or 2.

In a certain embodiment, Y² is O or NR².

In a certain embodiment, R² is hydrogen, C₁₋₆ alkyl, —C(═O)R^{c1} or —C(═O)OR^{c2}; R^{c1} is C₁₋₂₀ alkyl or C₁₋₂₀ alkyl substituted with one or more R⁴⁻¹⁻¹; R^{c2} is C₁₋₆ alkyl or C₁₋₆ alkyl substituted with one or more R⁴⁻¹⁻¹; R⁴⁻¹⁻¹ is independently halogen, C₆₋₂₀ aryl or —OC(═O)R²⁴; or, optional R² and R⁵ together with the atoms to which they are attached, independently form “5- to 12-membered heterocyclic alkenyl containing 2 to 4 heteroatoms, two of which are N, and the other heteroatoms independently selected from O, S, and N”.

In a certain embodiment, in R^{c}, R⁴⁻¹⁻¹ is independently halogen.

In a certain embodiment, in R^{c2}, R⁴⁻¹⁻¹ is independently C₆₋₂₀ aryl or —OC(═O)R²⁴.

In a certain embodiment, R²⁴ is C₁₋₆ alkyl.

In a certain embodiment, R⁴ is independently halogen, hydroxyl, deuterium, —C(═O)R^{4a}, O═ or C₁₋₆ alkyl; R^{4a} is C₁₋₆ alkyl or C₂₋₆ alkenyl.

In a certain embodiment, L₁ is a bond or —O(R^{L-1})ₙ₁—; R^{L-1} is C₁₋₆ alkylene or C₁₋₆ alkylene substituted with one or more deuterium; n1 is 1.

In a certain embodiment, R³ is C₃₋₁₂ cycloalkyl substituted with one or more R³⁻¹, “4- to 12-membered heterocycloalkyl containing 1-3 heteroatoms independently selected from O, S and N”, “4- to 12-membered heterocycloalkyl containing 1-3 heteroatoms independently selected from O, S and N” substituted with one or more R³⁻² or C₂₋₆ alkenyl substituted with one or more R³⁻⁴; provided that when multiple substituents are present, the substituents are the same or different.

In a certain embodiment, R³⁻¹ is independently C₁₋₆ alkyl substituted with one or more R³⁻¹⁻¹; R³⁻¹⁻¹ is independently —NR^{e10}R^{e}ii, “4- to 10-membered heterocycloalkyl containing 1-3 heteroatoms independently selected from O, S and N”, or, “4- to 10-membered heterocycloalkyl containing 1-3 heteroatoms independently selected from O, S and N” substituted with one or more R³⁻¹⁻¹⁻¹; R^{e10} and R^{e11} are independently C₁₋₆ alkyl; R³⁻¹⁻¹⁻¹ is independently

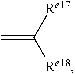

C₁₋₆ alkyl or C₆₋₂₀ aryl substituted with one or more R³⁻¹⁻¹⁻¹⁻¹; R³⁻¹⁻¹⁻¹⁻¹ is independently halogen or C₁₋₆ alkyl substituted with one or more halogen; R^{e7} and R^{e8} are independently hydrogen or halogen; or, R³⁻¹ is -L₂-M; L₂ is -L₂₁-L₂₂-L₂₃-L₂₄-L₂₅-L₂₆; L₂₁, L₂₃ and L₂₅ are independently a bond, O, S, NH, C(O)NH or C₁₋₆ alkylene; L₂₂ and L₂₄ are independently a bond, C₃₋₁₀ cycloalkyl, “4- to 7-membered heterocycloalkyl containing 1 or 2 heteroatoms independently selected from O, S and N”, C₆₋₂₀ aryl, or, “5- to 7-membered heteroaryl containing 1 or 2 heteroatoms independently selected from O, S, and N”; L₂₆ is a bond, O, S, NH or C(O)NH;
M is

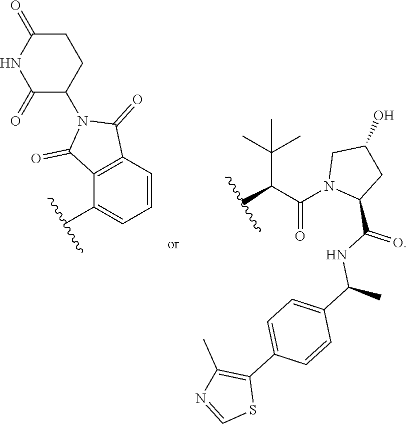 [SMILES: *c1cccc2c1C(=O)N(C1CCC(=O)NC1=O)C2=O ; *[C@H](C(=O)N1C[C@H](O)C[C@H]1C(=O)N[C@@H](C)c1ccc(-c2scnc2C)cc1)C(C)(C)C]

In a certain embodiment, R³⁻² is independently halogen, C₁₋₆ alkyl,

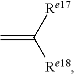

C₁₋₆ alkyl substituted with one or more R³⁻¹⁻¹, Ar, —CH₂—O—Ar or C₆₋₂₀ aryl substituted with one or more R³⁻¹⁻⁵; Ar is “5-12-membered heteroaryl containing 1-3 heteroatoms independently selected from O, N and S” substituted with one or more R³⁻¹⁻⁴; R³⁻¹⁻¹ is halogen, C₂₋₆ alkenyl or —OC(═O)NR^{e15}R^{e16}; R^{e15} and R^{e16} are independently C₁₋₆ alkyl; R^{e17} and R^{e18} are independently hydrogen or halogen; R³⁻¹⁻⁵ and R³⁻¹⁻⁴ are independently C₁₋₆ alkyl, halogen, C₁₋₆ alkyl-O— substituted with one or more halogen or C₁₋₆ alkyl substituted with one or more halogen; or, two optional R³⁻¹⁻⁵ are connected, together with the atoms to which they are attached form “4- to 10-membered heterocycloalkyl containing 1-3 heteroatoms independently selected from O, S and N” substituted with one or more R³⁻¹⁻¹⁻²; R³⁻¹⁻¹⁻² is independently halogen.

In a certain embodiment, R³⁻⁴ is independently halogen or C₁₋₆ alkyl substituted with one or more R³⁻¹⁻¹; R³⁻¹⁻¹ is independently —NR^{e10}R^{e11} or “4- to 10-membered heterocycloalkyl containing 1-3 heteroatoms independently selected from O, S and N”; R^{e10} and R^{e11} are independently C₁₋₆ alkyl.

In a certain embodiment, R^{e17} and R^{e18} are independently hydrogen or halogen.

In a certain embodiment, when the definition of R⁵, R^{b}, R¹⁻¹, R¹⁻², R¹⁻¹⁻¹, R¹⁻¹⁻², R¹⁻¹⁻³, R¹⁻¹⁻⁴, R¹⁻¹⁻⁵, R¹⁻¹⁻⁶, R⁴⁻¹⁻¹, R⁴⁻¹⁻², R⁴⁻¹⁻³, R⁴⁻¹⁻⁴, R⁴⁻¹⁻⁵, R⁴, R⁴⁻¹, R⁴⁻², R⁴⁻³, R⁴⁻⁴, R⁴⁻⁵, R^{a}, R³⁻¹, R³⁻², R³⁻³, R³⁻⁴, R³⁻¹⁻¹, R³⁻¹⁻², R³⁻¹⁻³, R³⁻¹⁻⁵, R³⁻¹⁻⁴, R^{4g}, R^{4h}, R^{e17}, R^{e18}, R³⁻¹⁻¹⁻¹ and R³⁻¹⁻¹⁻¹⁻¹ mentions halogen, the halogen is fluorine, chlorine, bromine or iodine.

In a certain embodiment, when the definition of R¹, R¹⁻¹, R¹⁻², R^{c1}, R^{c2}, R^{c3}, R^{c4}, R^{c5}, R^{c6}, R^{c7}, R^{c8}, R^{c9}, R¹⁻¹⁻¹, R¹⁻¹⁻², R¹⁻¹⁻³, R¹⁻¹⁻⁴, R¹⁻¹⁻⁵, R¹⁻¹⁻⁶, R⁴⁻¹⁻¹, R⁴⁻¹⁻², R⁴⁻¹⁻³, R⁴⁻¹⁻⁴, R⁴⁻¹⁻⁵, R^{c10}, R³⁻¹, R³⁻², R³⁻³, R³⁻⁴ and R³⁻¹⁻¹⁻¹ mentions C₆₋₂₀ aryl, the C₆₋₂₀ aryl is phenyl or naphthyl.

In a certain embodiment, when the definition of R⁵, R¹⁻¹, R¹⁻², R², R^{c}, R¹², R¹³, R^{c10}, R^{c10-1}, R¹⁻¹⁻¹, R¹⁻¹⁻², R¹⁻¹⁻³, R¹⁻¹⁻⁴, R¹⁻¹⁻⁵R¹⁻¹⁻⁶, R⁴⁻¹⁻¹R⁴⁻¹⁻²R⁴⁻¹⁻³, R⁴⁻¹⁻⁴, R⁴⁻¹⁻⁵, R^{c51}, R^{c52}, R¹¹, R²¹, R²², R²³, R¹⁴, R²⁴, R¹⁵, R², R¹⁶, R²⁶, R²⁷, R²⁸, R²⁹, R³⁰, R³¹, R³², R³³, R^{x}, R^{y}, R⁴, R⁴⁻¹, R⁴⁻², R⁴⁻³, R⁴⁻⁴, R⁴⁻⁵, R^{a}, R^{4a}, R^{4b1}, R^{4b2}, R^{4c}, R^{4d1}, R^{4d2}, R^{4e}, R^{4f1}, R^{4f2}, R⁴ⁱ, R^{4j}, R^{L-3}, R³, R³⁻¹, R³⁻², R³⁻³, R³⁻⁴, R^{4g}, R^{4h}, R^{e17}, R^{e18}, R^{d}, R^{d1}, R^{e1}, R^{e2}, R^{e3}, R^{e4}, R³⁻¹⁻¹, R³⁻¹⁻², R³⁻¹⁻⁵, R³⁻¹⁻⁴, R³⁻¹⁻¹⁻¹, R³⁻¹⁻¹⁻¹⁻¹, R^{e5}, R^{e6}, R^{e7}, R^{e8}, R^{e9}, R^{e10}, R^{e11}, R^{e12}, R^{e13}, Re¹⁴, R^{e15}, R^{e16}, R^{e171}, R^{e172}, R^{e173}, R^{e174} and R³⁻¹⁻³ mentions C₁₋₆ alkyl, the C₁₋₆ alkyl is C₁₋₄ alkyl, for example, methyl, ethyl, n-propyl, isopropyl, n-butyl, sec-butyl, isobutyl or tert-butyl.

In a certain embodiment, when the definition of R^{c1}, R^{c2}, R^{c3}, R^{c4}, R^{c5}, R^{c6}, R^{c7}, R^{c8} and R^{d9} independently is C₁₋₂₀ alkyl, the C₁₋₂₀ alkyl is C₁₋₁₂ alkyl, for example, methyl, ethyl, n-propyl, isopropyl, n-butyl, sec-butyl, isobutyl or tert-butyl.

In a certain embodiment, when the definition of R¹⁻¹, R¹⁻² and R⁴ mentions C₂₋₆ alkynyl, the C₂₋₆ alkynyl is C₂₋₃ alkynyl, for example, ethynyl, propynyl or propargyl.

In a certain embodiment, when the definition of R⁵, R¹⁻¹, R¹⁻², R⁴, R⁴⁻¹, R⁴⁻², R⁴⁻³, R⁴⁻⁴, R⁴-⁵, R^{a}, R^{4a}, R^{4b1}, R^{4b2}, R^{4c}, R^{4d1}, R^{4d2}, R^{4e}, R^{4f1}, R^{4f2}, R⁴ⁱ, R^{4j}, R³, R³⁻¹⁻¹, R³⁻¹⁻², R³⁻¹⁻⁵ and R³⁻¹⁻⁴ mentions C₂₋₆ alkenyl, the C₂₋₆ alkenyl is C₂₋₃ alkenyl, for example, vinyl, propenyl or allyl.

In a certain embodiment, when m1 is 0, m2 is 1, or m1 is 1, m2 is 0, n is 1 or 2.

In a certain embodiment, when m1 and m2 are 1, n is 0.

In a certain embodiment, when R¹ is “5- to 12-membered heteroaryl containing 1-4 heteroatoms independently selected from O, S and N” substituted with one or more R¹⁻², the “5- to 12-membered heteroaryl containing 1-4 heteroatoms independently selected from O, S and N” is “8- to 10-membered heteroaryl containing 1 heteroatom selected from O, S and N”, for example, benzothiophenyl.

In a certain embodiment, when Ar is “5-12-membered heteroaryl containing 1-3 heteroatoms independently selected from O, N and S” substituted with one or more R³⁻¹⁻⁴, the “5-12-membered heteroaryl containing 1-3 heteroatoms independently selected from O, N and S” is “5-10-membered heteroaryl containing 1-2 heteroatoms being N”, for example, pyridazinyl or pyrimidinyl.

In a certain embodiment, when R³⁻¹⁻¹, R³⁻¹⁻⁵ and R³⁻¹⁻⁴ are independently C₁₋₆ alkyl substituted with one or more halogen, the C₁₋₆ alkyl substituted with one or more halogen is C₁₋₂ alkyl substituted with two or three fluorine.

In a certain embodiment, R⁴ is independently methyl, F, D, oxo

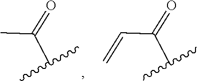

hydroxyl.

In a certain embodiment,

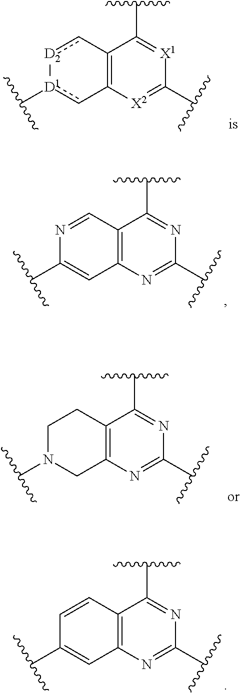

In a certain embodiment, R⁵ is independently —F, —OH, —Cl, —CF₃,

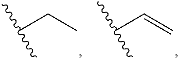

—NO₂, -OEt or —OMe, or, R² and R⁵ form

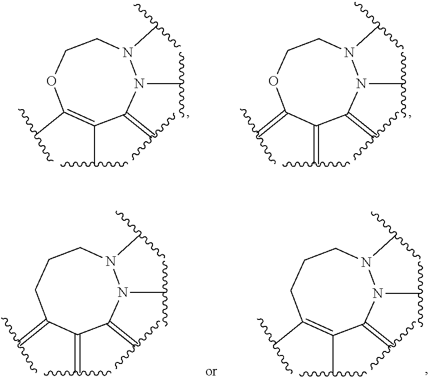 [SMILES: */C1=C(\*)C(=*)N(*)N(*)CCO1 ; *N1CCOC(=*)C(=*)C(=*)N1* ; *N1CCCC(=*)C(=*)C(=*)N1* ; */C1=C(\*)C(=*)N(*)N(*)CCC1]

In a certain embodiment, R¹ is

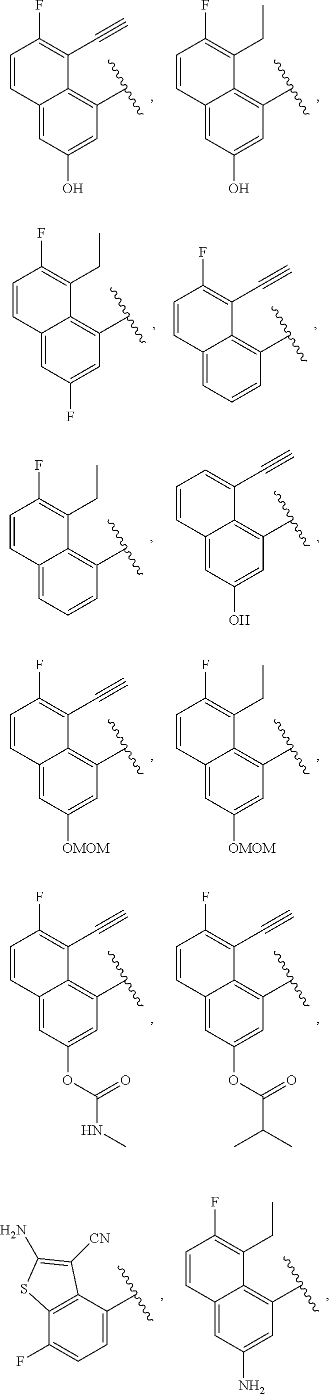

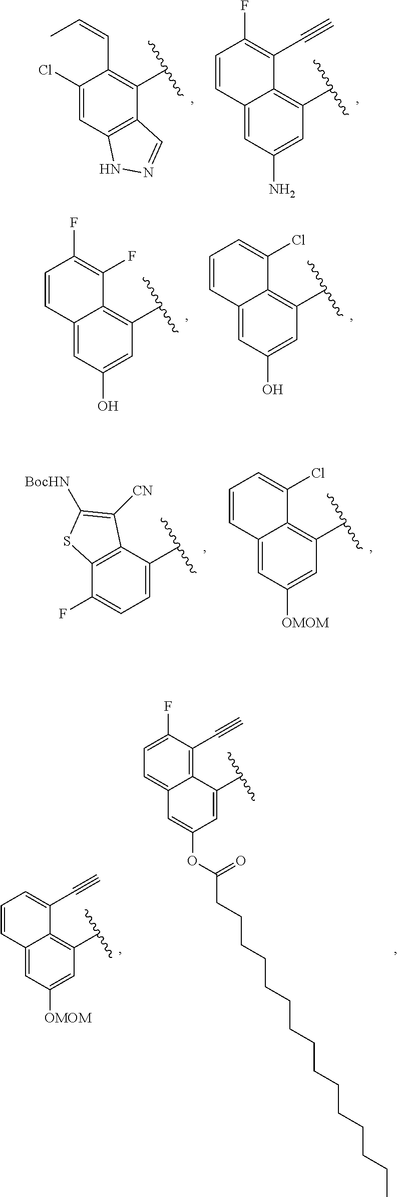

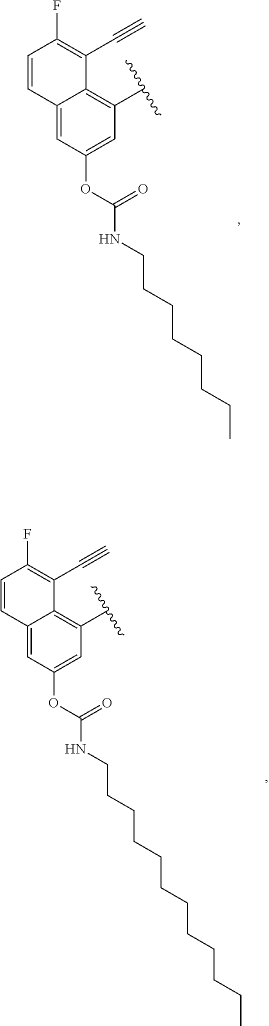 [SMILES: *c1cc(OC(=O)NCCCCCCCC)cc2ccc(F)c(C#C)c12 ; *c1cc(OC(=O)NCCCCCCCCCCCC)cc2ccc(F)c(C#C)c12]

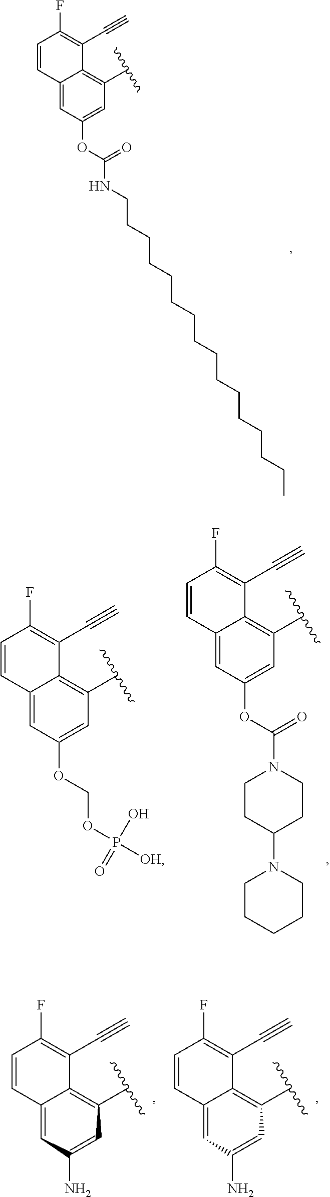

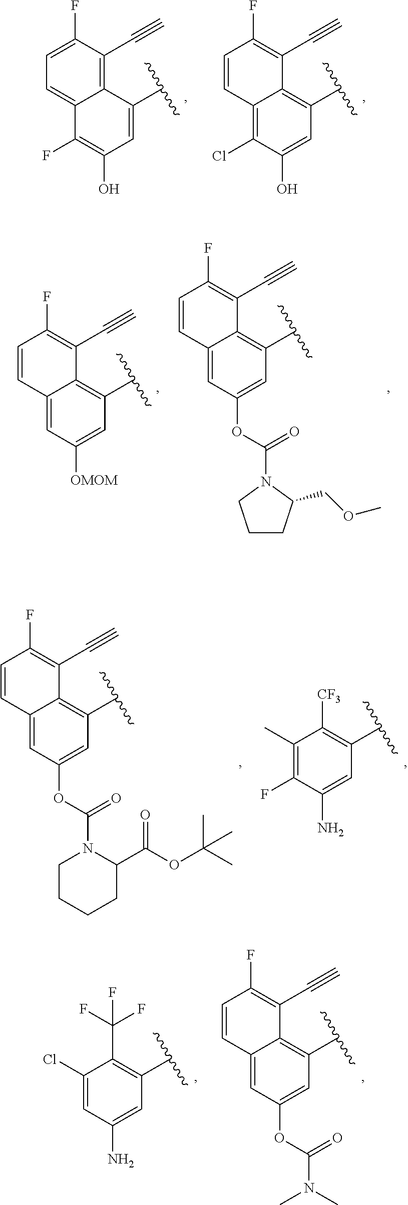

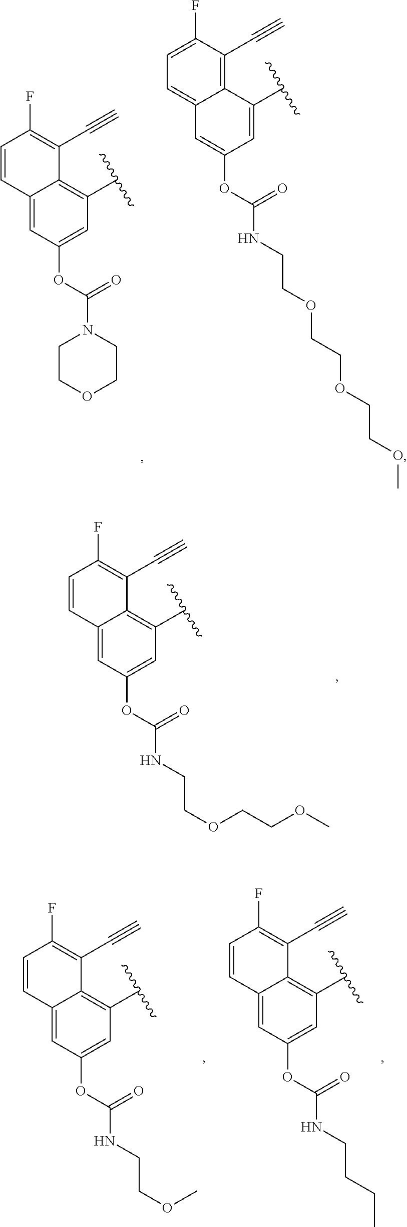 [SMILES: *c1cc(OC(=O)N2CCOCC2)cc2ccc(F)c(C#C)c12 ; *c1cc(OC(=O)NCCOCCOCCOC)cc2ccc(F)c(C#C)c12 ; *c1cc(OC(=O)NCCOCCOC)cc2ccc(F)c(C#C)c12 ; *c1cc(OC(=O)NCCOC)cc2ccc(F)c(C#C)c12 ; *c1cc(OC(=O)NCCCC)cc2ccc(F)c(C#C)c12]

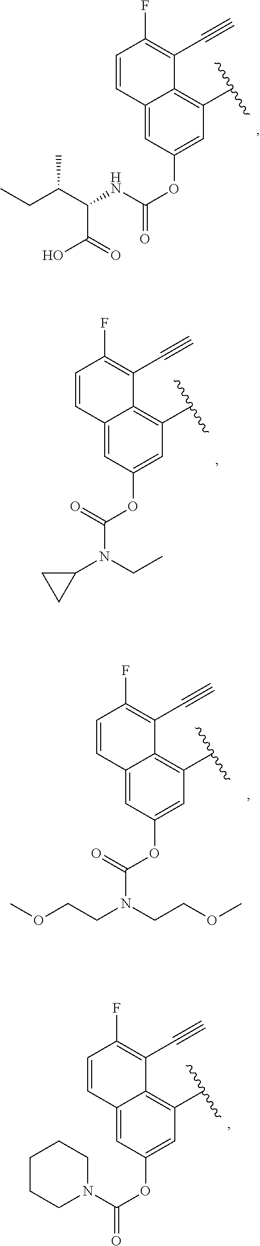

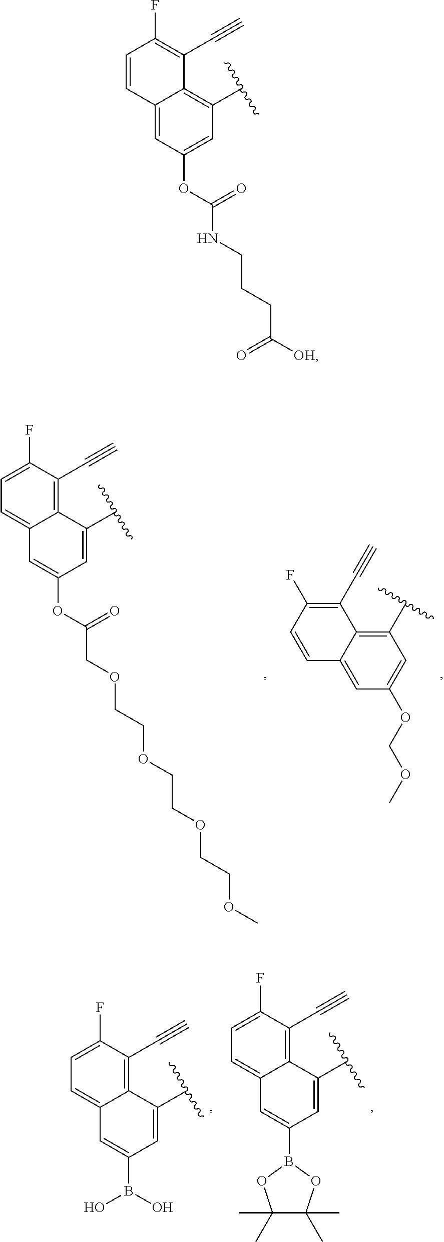

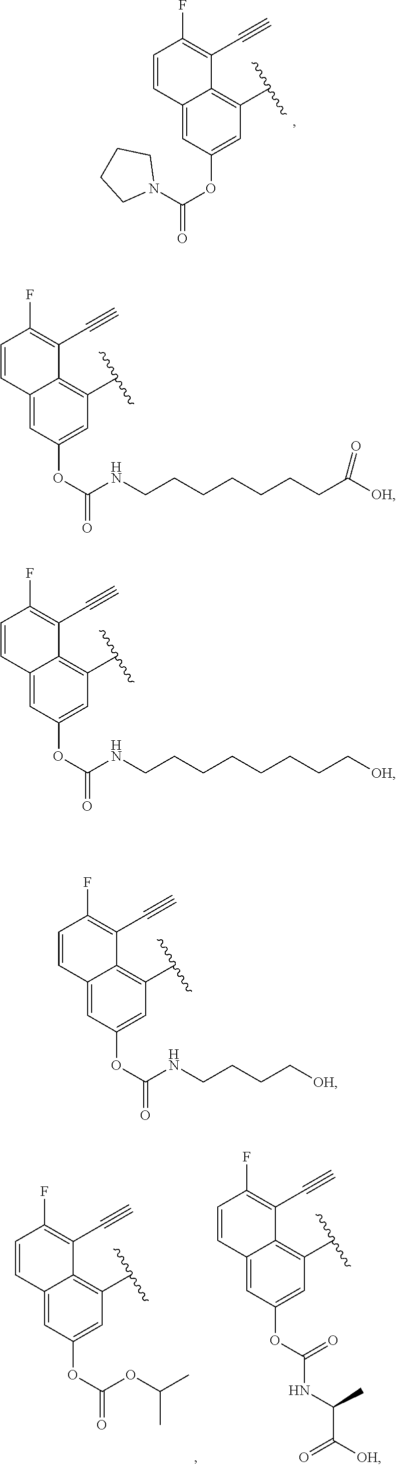 [SMILES: *c1cc(OC(=O)N2CCCC2)cc2ccc(F)c(C#C)c12 ; *c1cc(OC(=O)NCCCCCCCC(=O)O)cc2ccc(F)c(C#C)c12 ; *c1cc(OC(=O)NCCCCCCCCO)cc2ccc(F)c(C#C)c12 ; *c1cc(OC(=O)NCCCCO)cc2ccc(F)c(C#C)c12 ; *c1cc(OC(=O)OC(C)C)cc2ccc(F)c(C#C)c12 ; *c1cc(OC(=O)N[C@@H](C)C(=O)O)cc2ccc(F)c(C#C)c12]

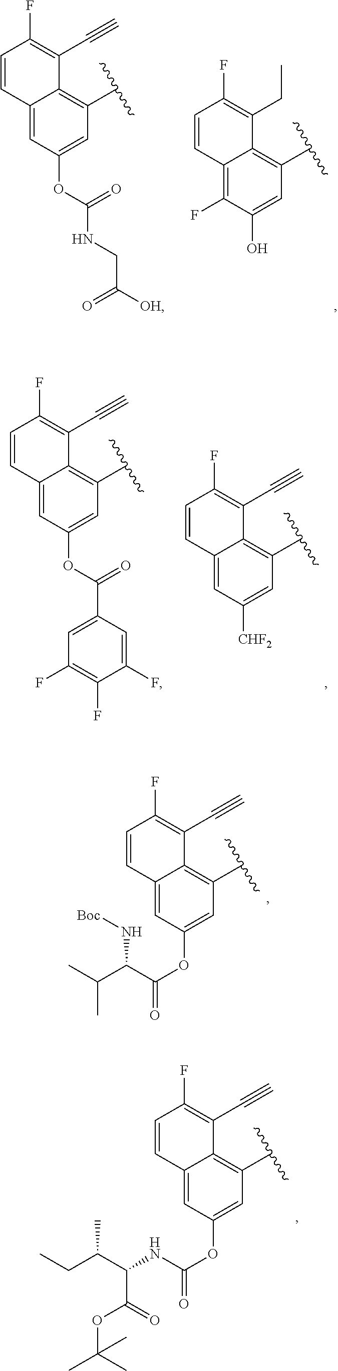 [SMILES: *c1cc(OC(=O)NCC(=O)O)cc2ccc(F)c(C#C)c12 ; *c1cc(O)c(F)c2ccc(F)c(CC)c12 ; *c1cc(OC(=O)c2cc(F)c(F)c(F)c2)cc2ccc(F)c(C#C)c12 ; *c1cc(C(F)F)cc2ccc(F)c(C#C)c12 ; *c1cc(OC(=O)[C@@H](NC(=O)OC(C)(C)C)C(C)C)cc2ccc(F)c(C#C)c12 ; *c1cc(OC(=O)N[C@H](C(=O)OC(C)(C)C)[C@@H](C)CC)cc2ccc(F)c(C#C)c12]

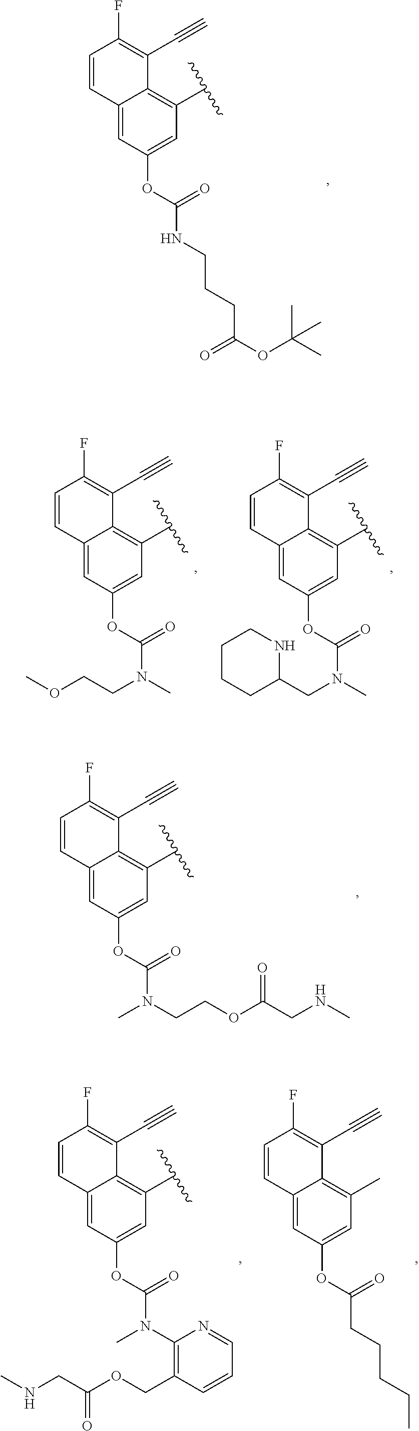

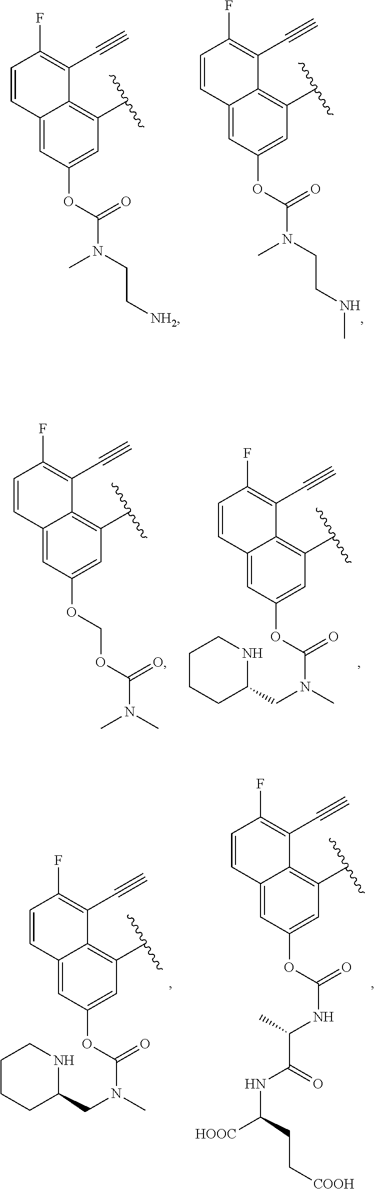

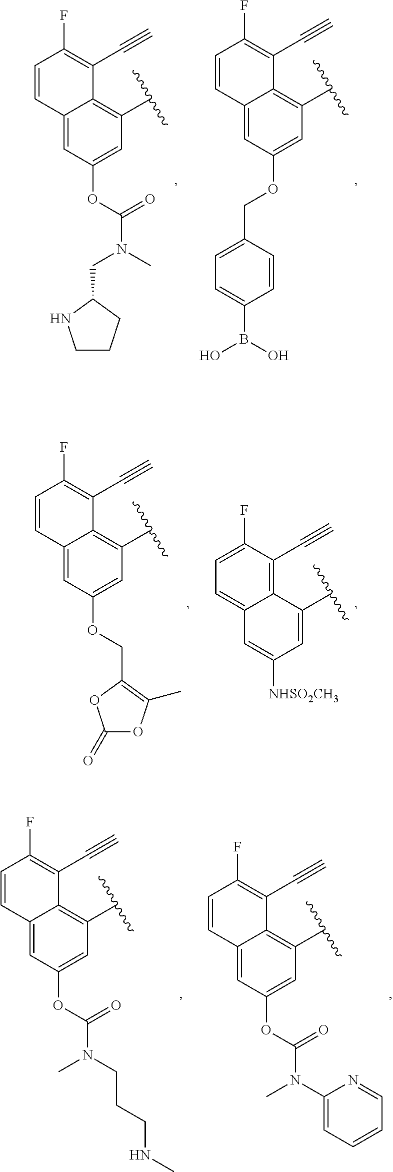

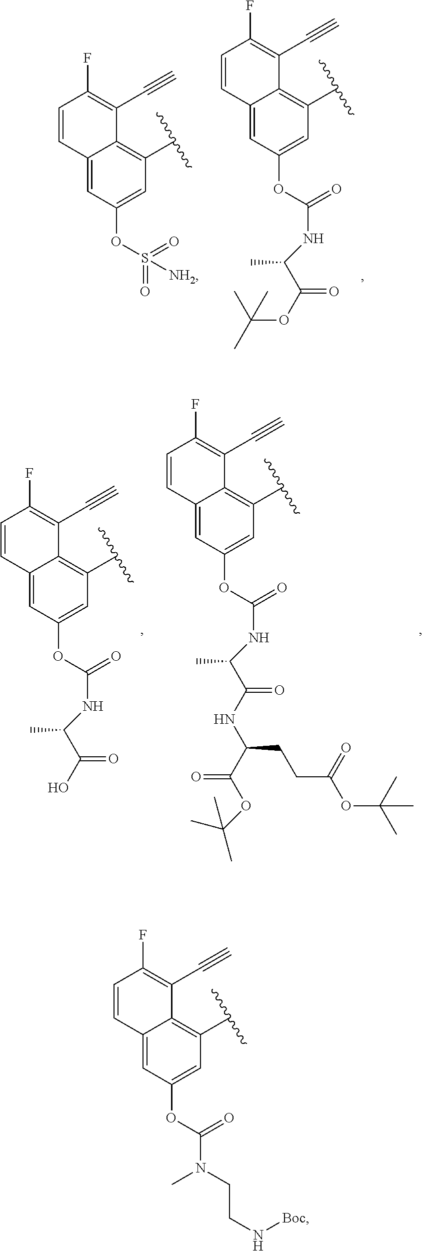 [SMILES: *c1cc(OS(N)(=O)=O)cc2ccc(F)c(C#C)c12 ; *c1cc(OC(=O)N[C@@H](C)C(=O)OC(C)(C)C)cc2ccc(F)c(C#C)c12 ; *c1cc(OC(=O)N[C@@H](C)C(=O)O)cc2ccc(F)c(C#C)c12 ; *c1cc(OC(=O)N[C@@H](C)C(=O)N[C@@H](CCC(=O)OC(C)(C)C)C(=O)OC(C)(C)C)cc2ccc(F)c(C#C)c12 ; *c1cc(OC(=O)N(C)CCNC(=O)OC(C)(C)C)cc2ccc(F)c(C#C)c12]

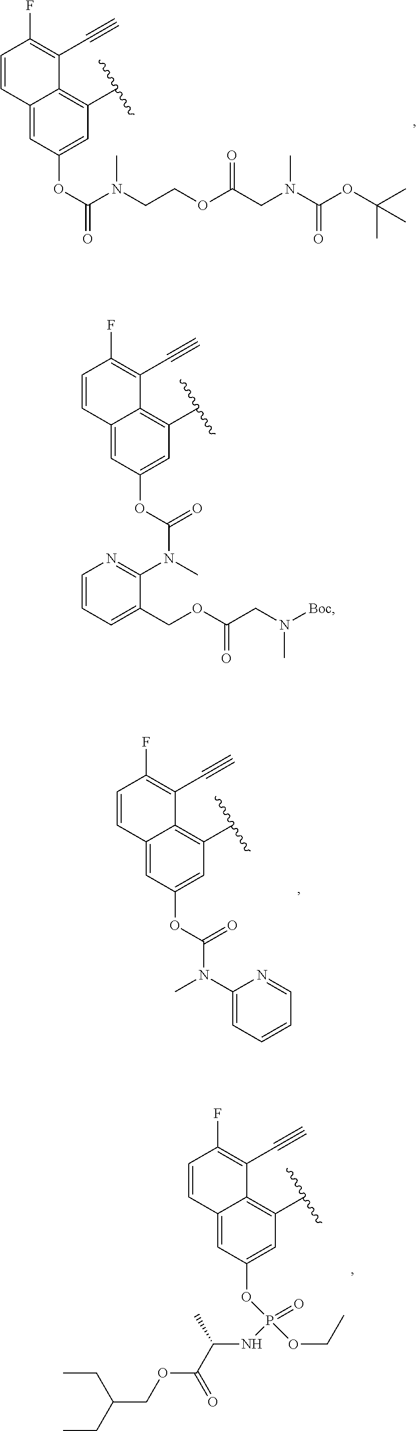

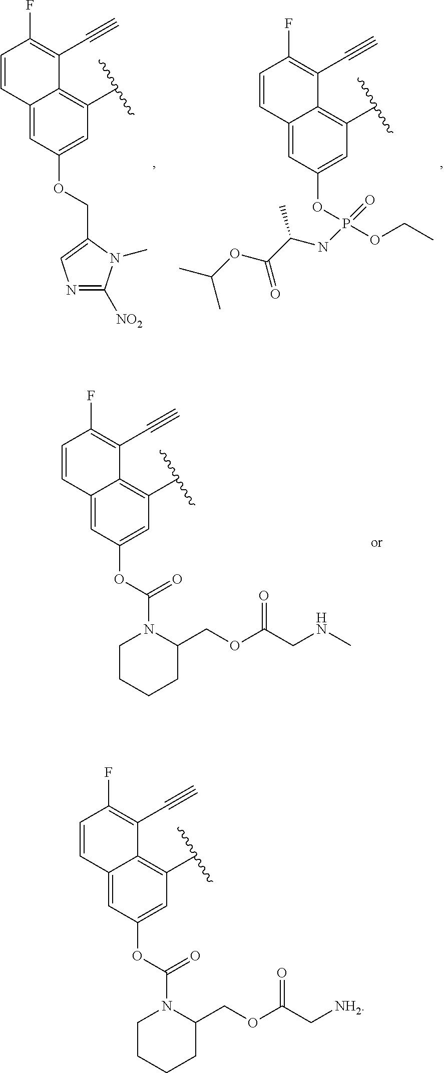

In a certain embodiment, L₁ is a bond, —OCH₂— or -OCD₂-.

In a certain embodiment, R³ is

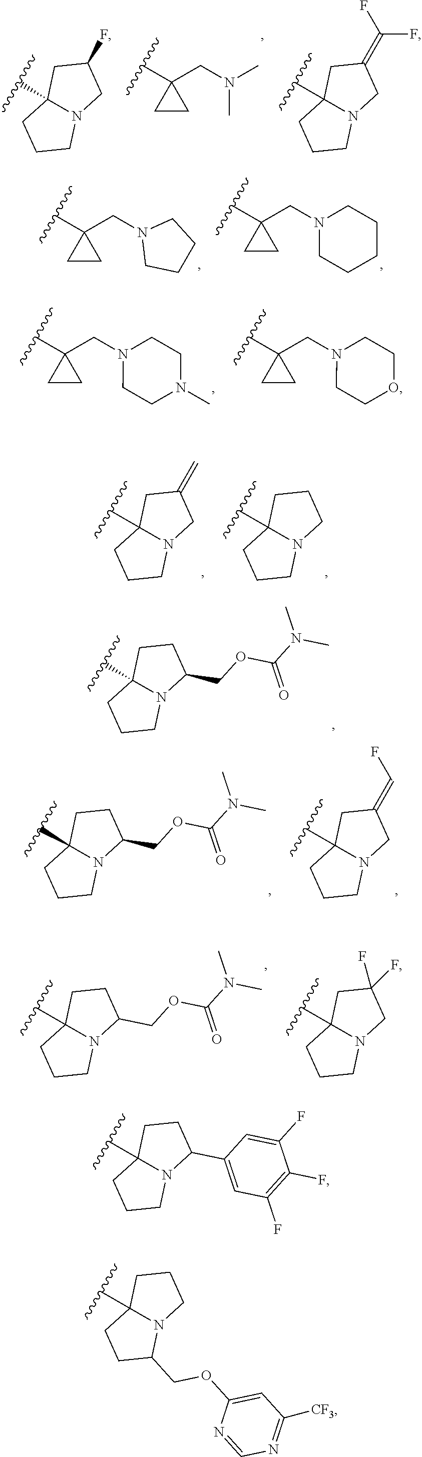

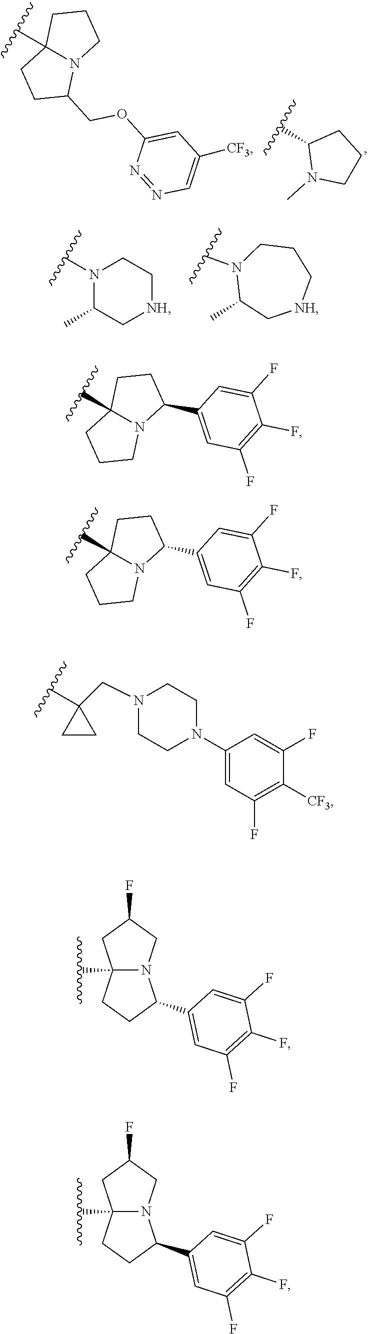

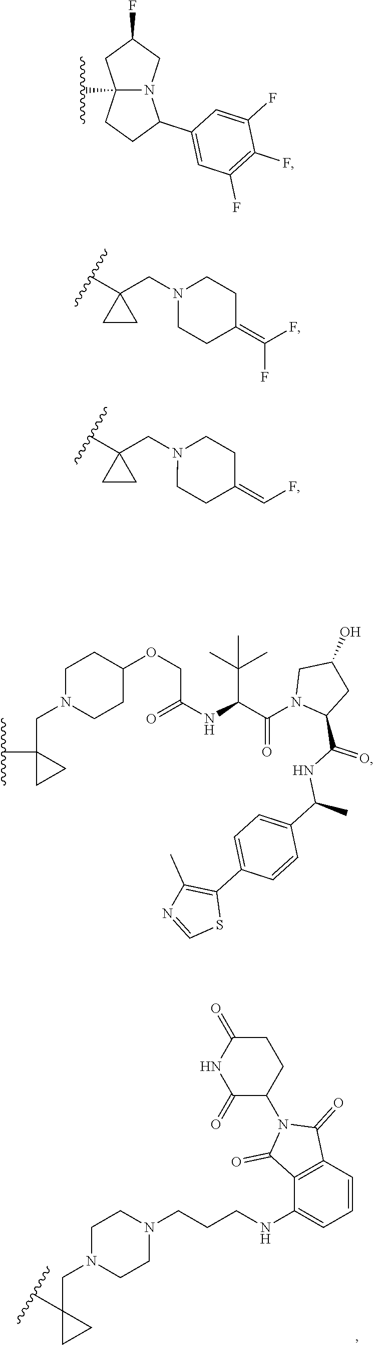 [SMILES: *[C@@]12CCC(c3cc(F)c(F)c(F)c3)N1C[C@H](F)C2 ; *C1(CN2CCC(=C(F)F)CC2)CC1 ; *C1(CN2CCC(=CF)CC2)CC1 ; *C1(CN2CCC(OCC(=O)N[C@H](C(=O)N3C[C@H](O)C[C@H]3C(=O)N[C@@H](C)c3ccc(-c4scnc4C)cc3)C(C)(C)C)CC2)CC1 ; *C1(CN2CCN(CCCNc3cccc4c3C(=O)N(C3CCC(=O)NC3=O)C4=O)CC2)CC1]

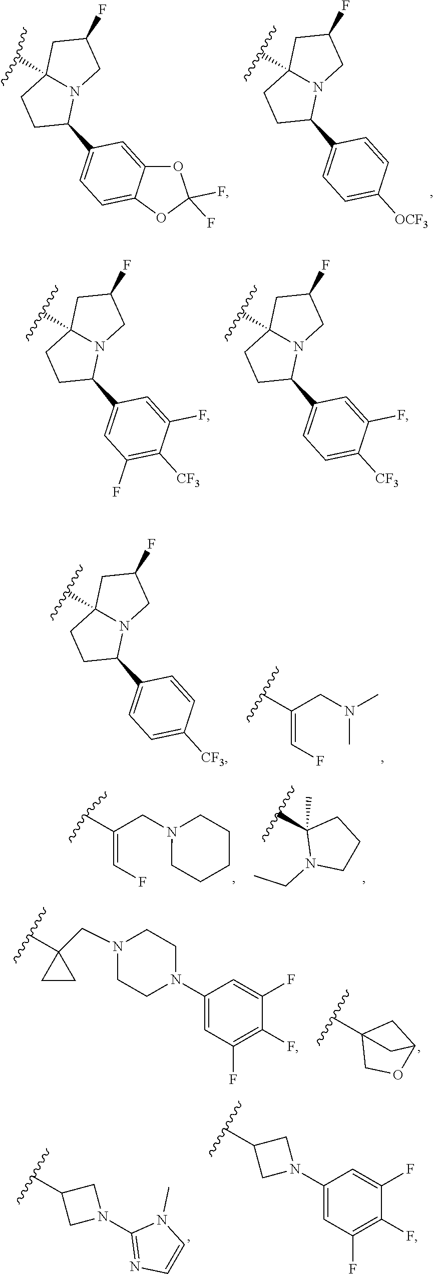

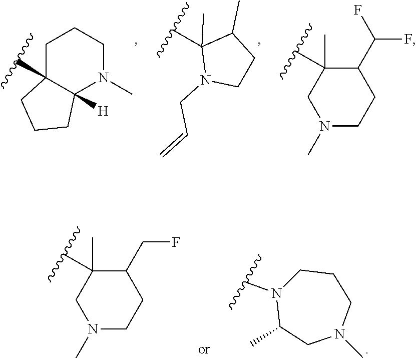

In a certain embodiment,

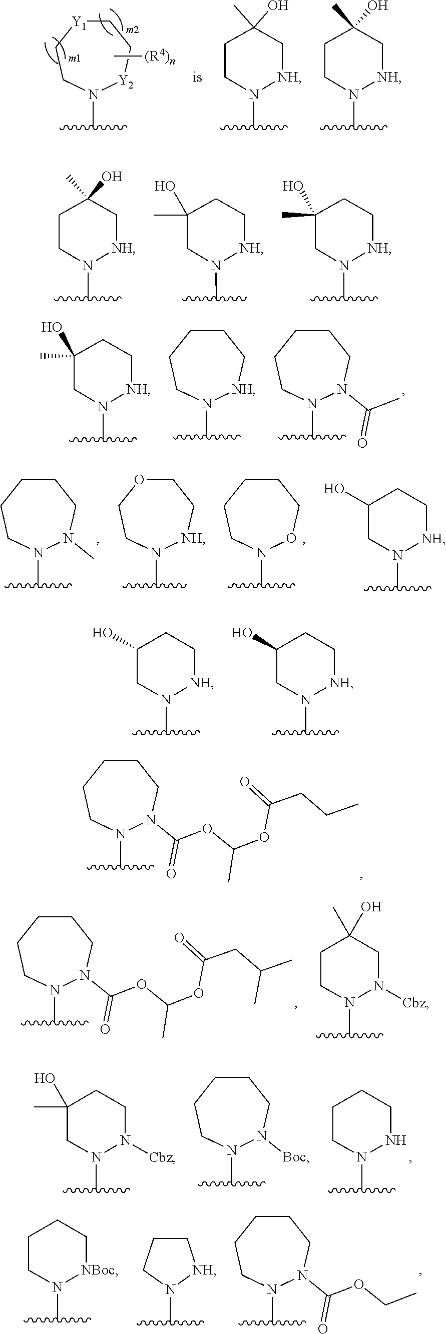

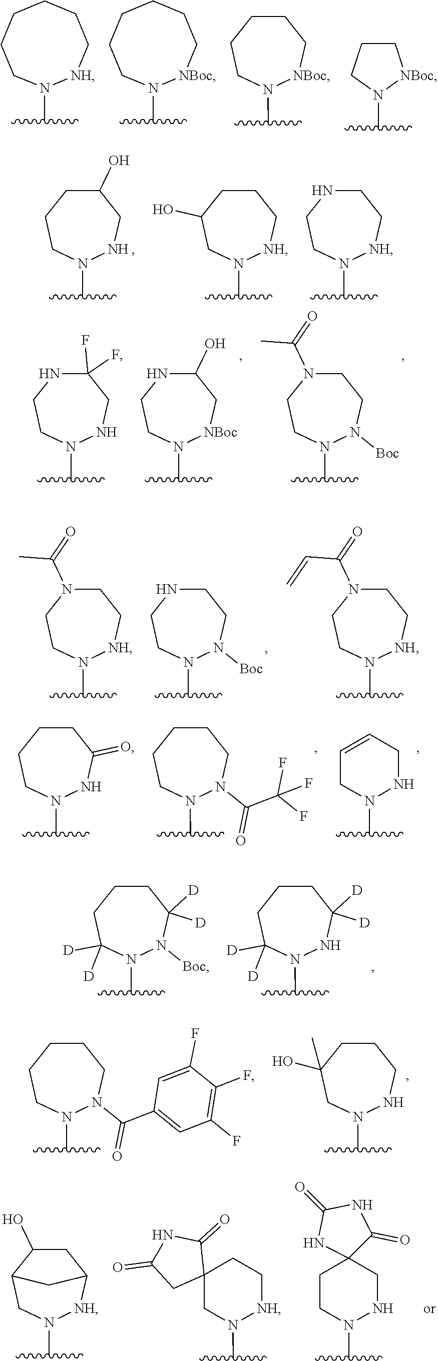

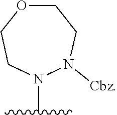 [SMILES: *N1CCOCCN1C(=O)OCc1ccccc1]

In a certain embodiment,

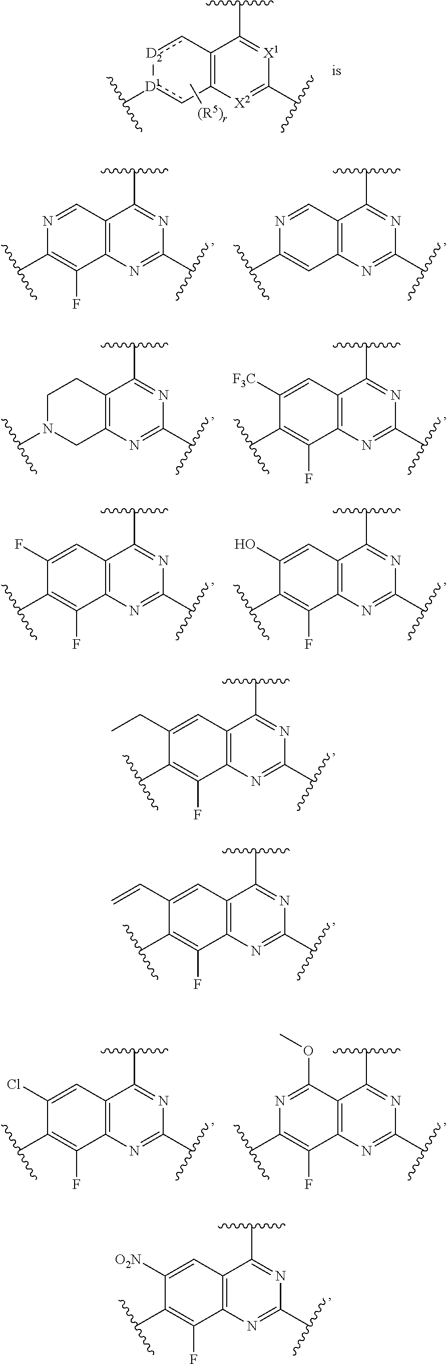

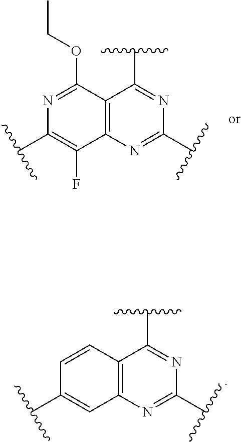 [SMILES: *c1nc(*)c2c(OCC)nc(*)c(F)c2n1 ; *c1ccc2c(*)nc(*)nc2c1]

In a certain embodiment, is

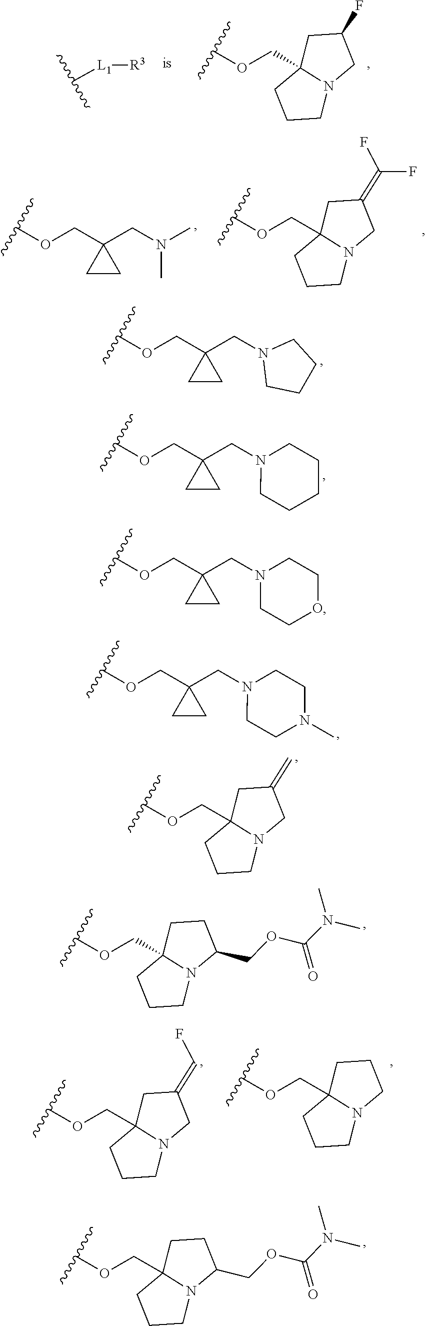

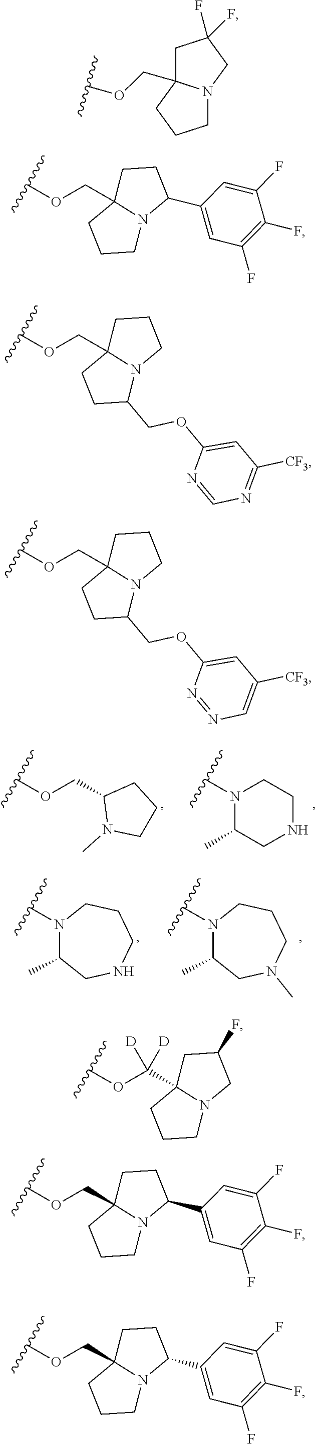

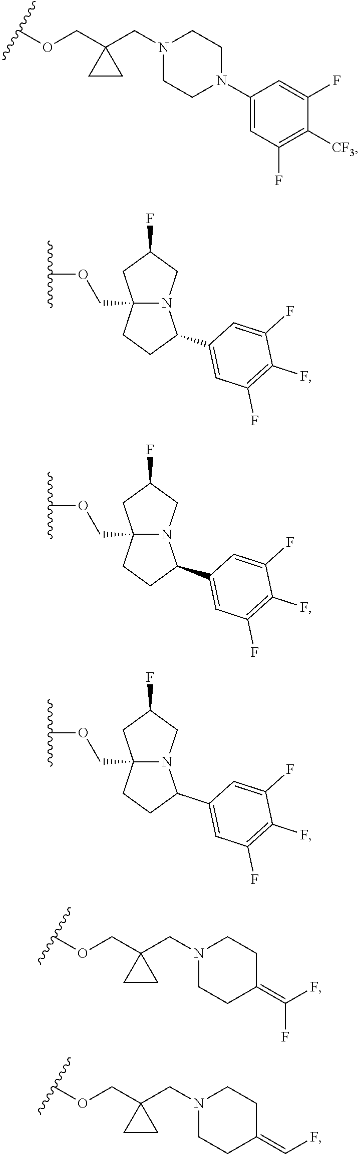 [SMILES: *OCC1(CN2CCN(c3cc(F)c(C(F)(F)F)c(F)c3)CC2)CC1 ; *OC[C@@]12CC[C@@H](c3cc(F)c(F)c(F)c3)N1C[C@H](F)C2 ; *OC[C@@]12CC[C@H](c3cc(F)c(F)c(F)c3)N1C[C@H](F)C2 ; *OC[C@@]12CCC(c3cc(F)c(F)c(F)c3)N1C[C@H](F)C2 ; *OCC1(CN2CCC(=C(F)F)CC2)CC1 ; *OCC1(CN2CCC(=CF)CC2)CC1]

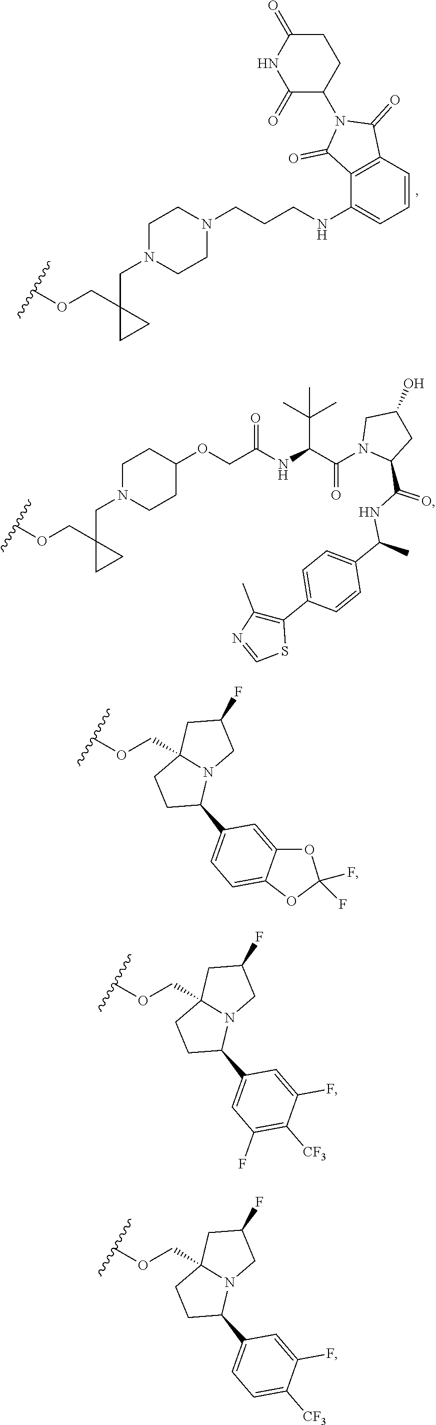

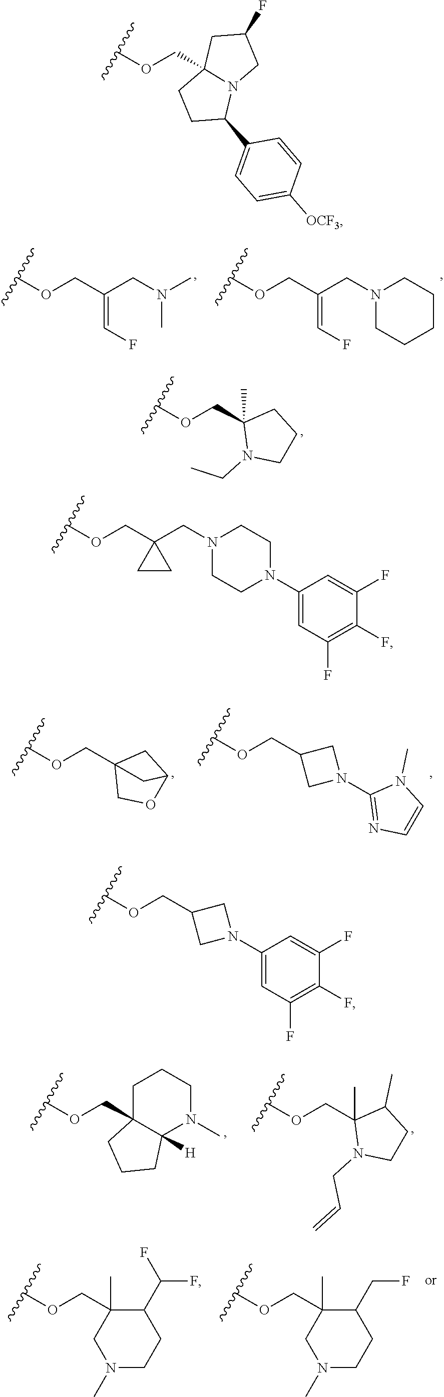

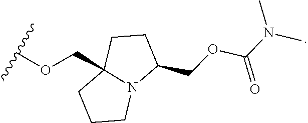 [SMILES: *OC[C@]12CCCN1[C@H](COC(=O)N(C)C)CC2]

In a certain embodiment, the nitrogen-containing heterocyclic compound of formula I has any of the following structures:

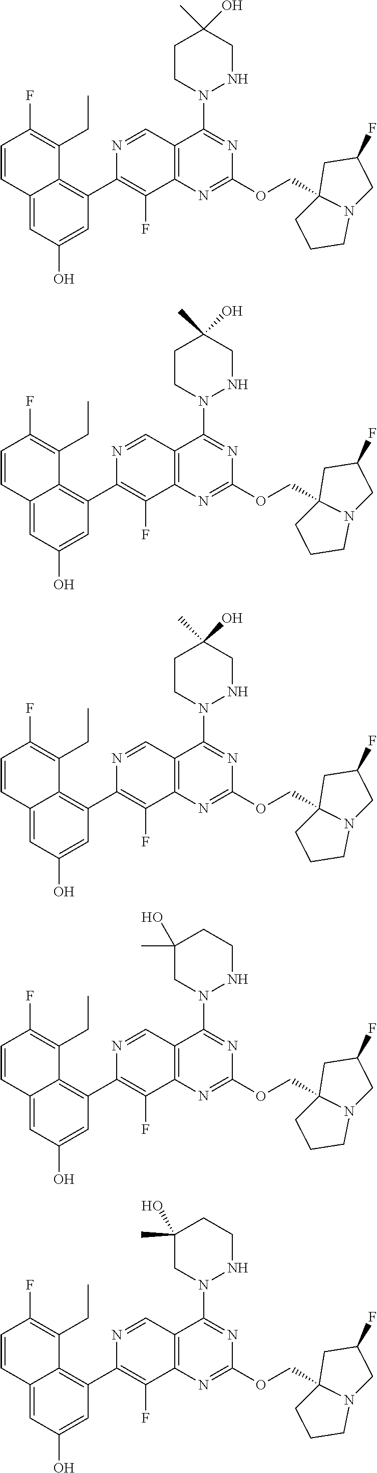 [SMILES: CCc1c(F)ccc2cc(O)cc(-c3ncc4c(N5CCC(C)(O)CN5)nc(OC[C@@]56CCCN5C[C@H](F)C6)nc4c3F)c12 ; CCc1c(F)ccc2cc(O)cc(-c3ncc4c(N5CC[C@](C)(O)CN5)nc(OC[C@@]56CCCN5C[C@H](F)C6)nc4c3F)c12 ; CCc1c(F)ccc2cc(O)cc(-c3ncc4c(N5CC[C@@](C)(O)CN5)nc(OC[C@@]56CCCN5C[C@H](F)C6)nc4c3F)c12 ; CCc1c(F)ccc2cc(O)cc(-c3ncc4c(N5CC(C)(O)CCN5)nc(OC[C@@]56CCCN5C[C@H](F)C6)nc4c3F)c12 ; CCc1c(F)ccc2cc(O)cc(-c3ncc4c(N5C[C@](C)(O)CCN5)nc(OC[C@@]56CCCN5C[C@H](F)C6)nc4c3F)c12]

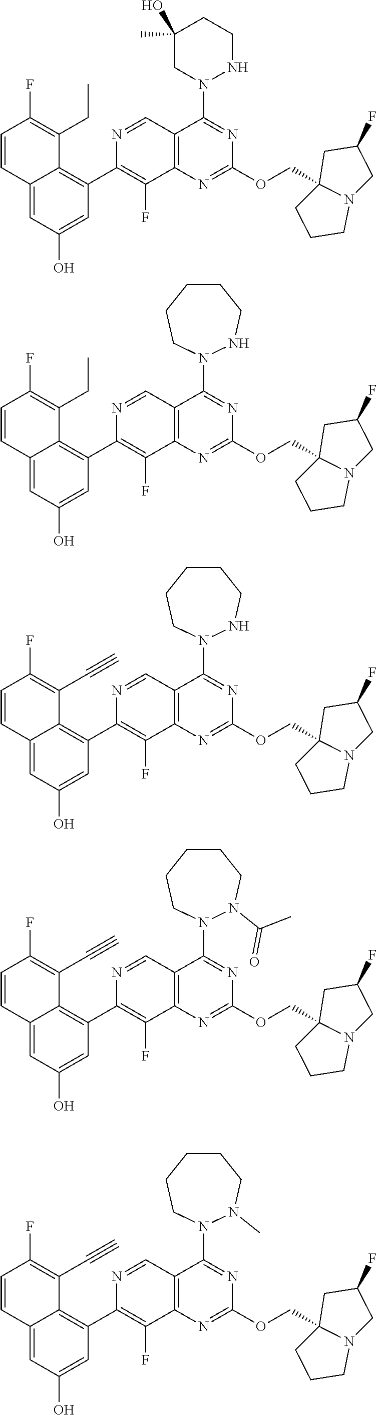 [SMILES: CCc1c(F)ccc2cc(O)cc(-c3ncc4c(N5C[C@@](C)(O)CCN5)nc(OC[C@@]56CCCN5C[C@H](F)C6)nc4c3F)c12 ; CCc1c(F)ccc2cc(O)cc(-c3ncc4c(N5CCCCCN5)nc(OC[C@@]56CCCN5C[C@H](F)C6)nc4c3F)c12 ; C#Cc1c(F)ccc2cc(O)cc(-c3ncc4c(N5CCCCCN5)nc(OC[C@@]56CCCN5C[C@H](F)C6)nc4c3F)c12 ; C#Cc1c(F)ccc2cc(O)cc(-c3ncc4c(N5CCCCCN5C(C)=O)nc(OC[C@@]56CCCN5C[C@H](F)C6)nc4c3F)c12 ; C#Cc1c(F)ccc2cc(O)cc(-c3ncc4c(N5CCCCCN5C)nc(OC[C@@]56CCCN5C[C@H](F)C6)nc4c3F)c12]

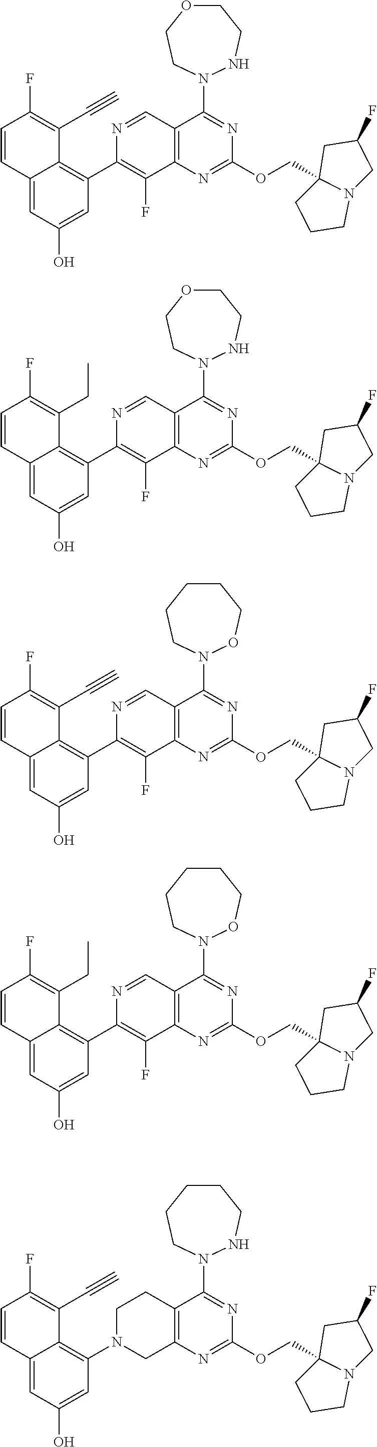 [SMILES: C#Cc1c(F)ccc2cc(O)cc(-c3ncc4c(N5CCOCCN5)nc(OC[C@@]56CCCN5C[C@H](F)C6)nc4c3F)c12 ; CCc1c(F)ccc2cc(O)cc(-c3ncc4c(N5CCOCCN5)nc(OC[C@@]56CCCN5C[C@H](F)C6)nc4c3F)c12 ; C#Cc1c(F)ccc2cc(O)cc(-c3ncc4c(N5CCCCCO5)nc(OC[C@@]56CCCN5C[C@H](F)C6)nc4c3F)c12 ; CCc1c(F)ccc2cc(O)cc(-c3ncc4c(N5CCCCCO5)nc(OC[C@@]56CCCN5C[C@H](F)C6)nc4c3F)c12 ; C#Cc1c(F)ccc2cc(O)cc(N3CCc4c(nc(OC[C@@]56CCCN5C[C@H](F)C6)nc4N4CCCCCN4)C3)c12]

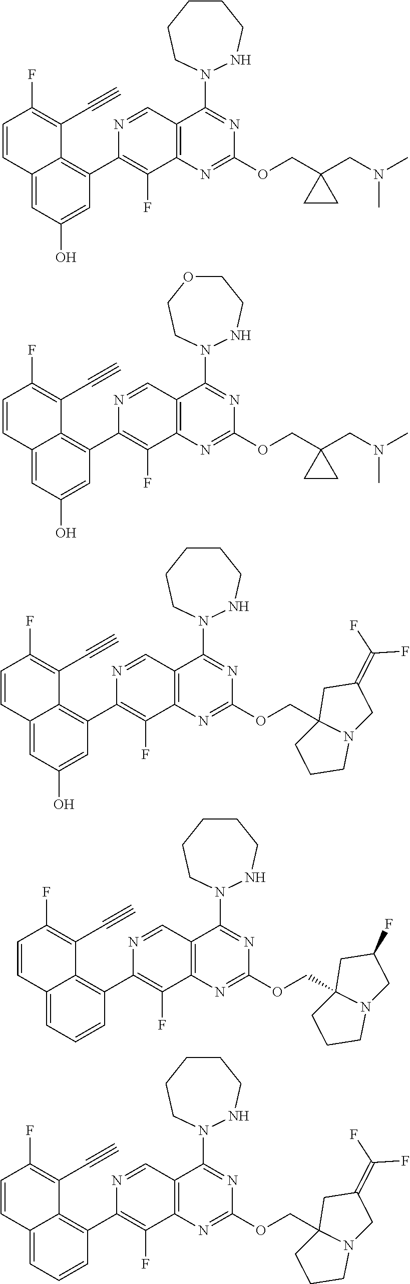

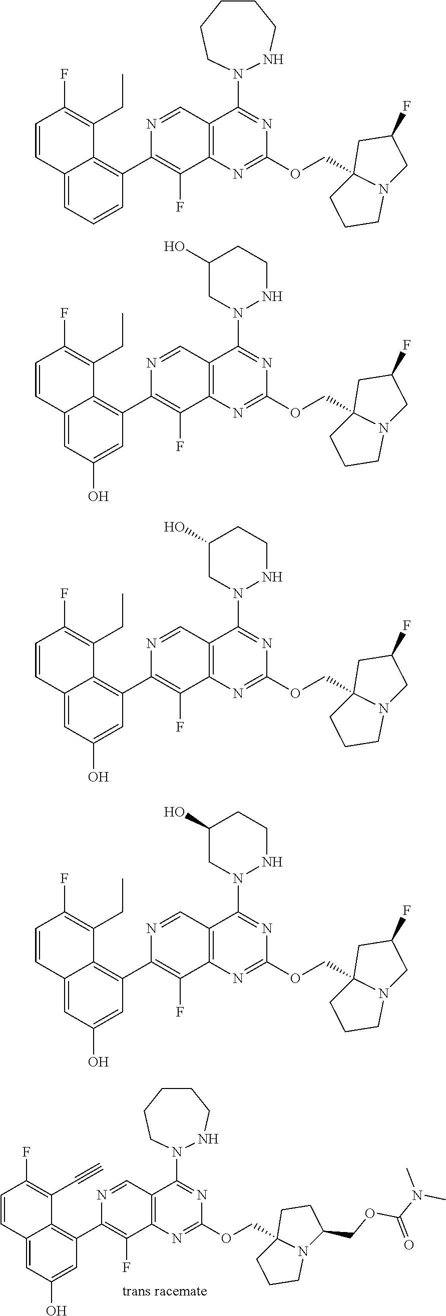

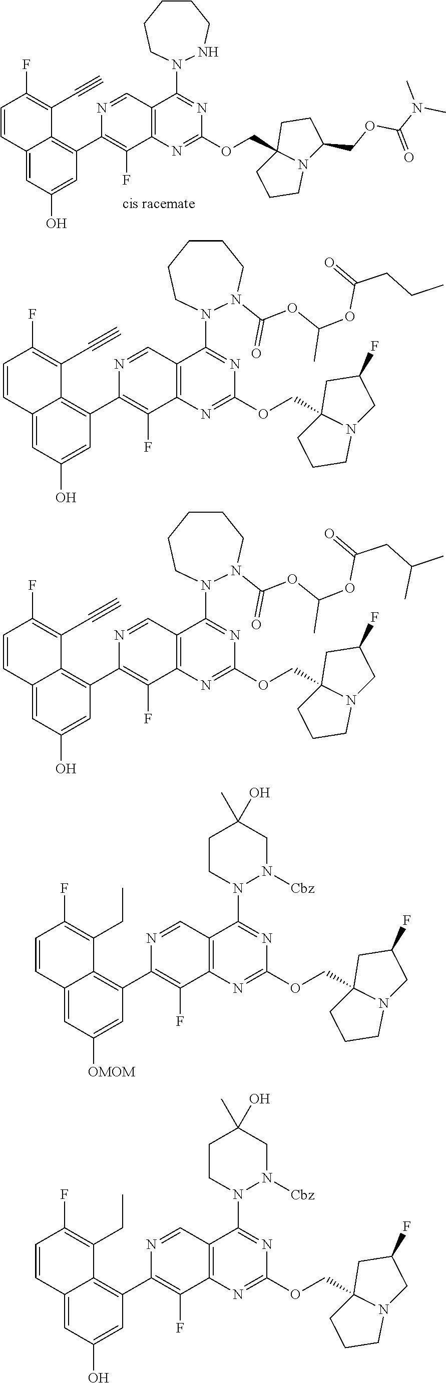

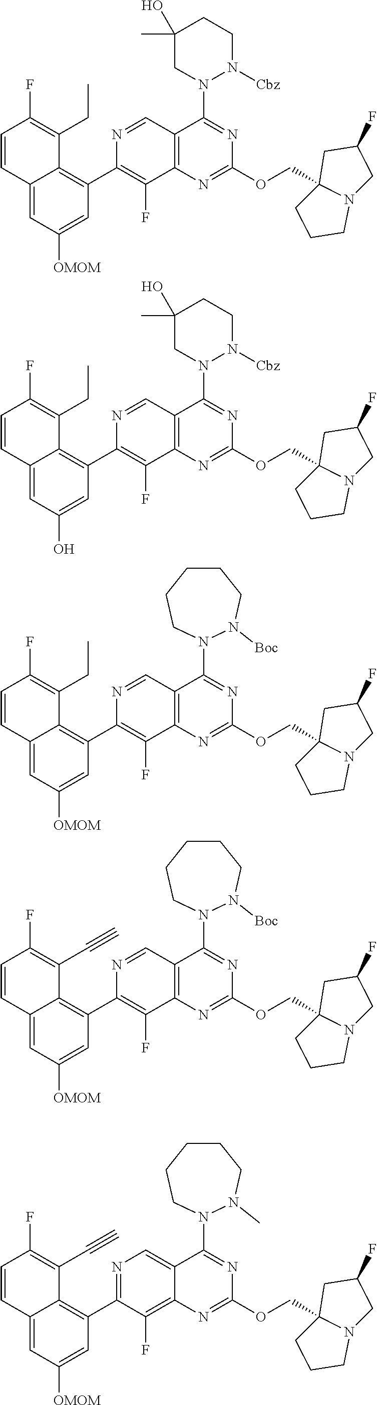

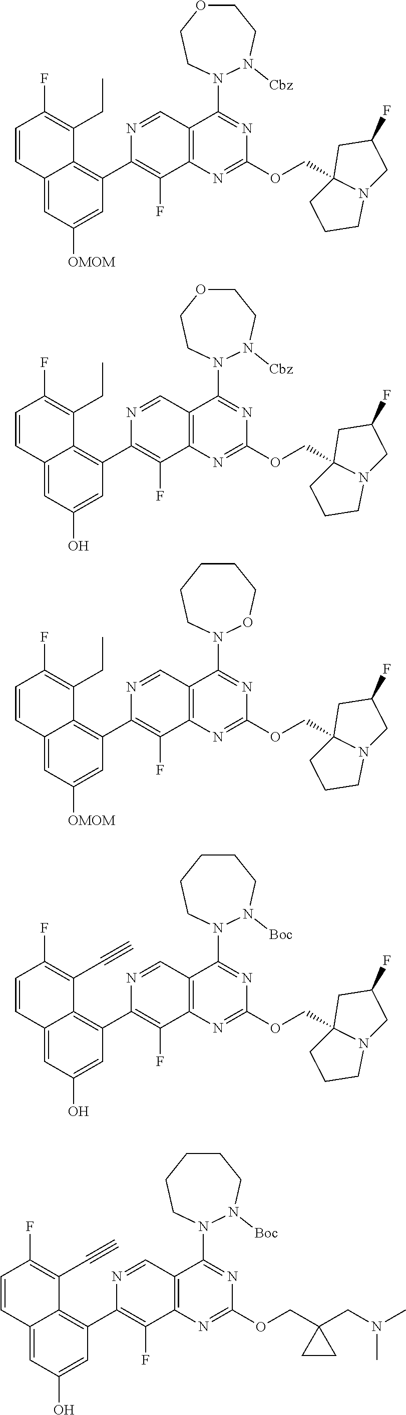

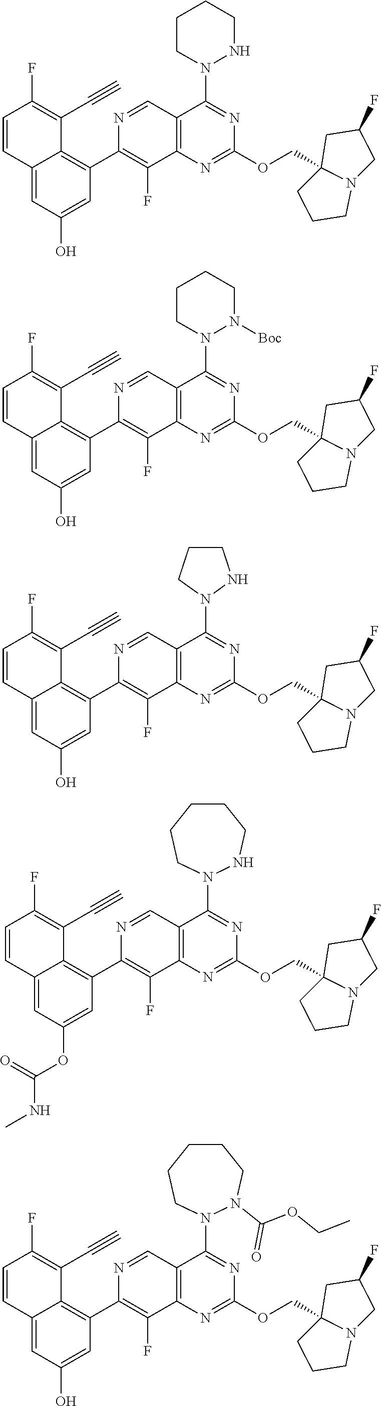 [SMILES: C#Cc1c(F)ccc2cc(O)cc(-c3ncc4c(N5CCCCN5)nc(OC[C@@]56CCCN5C[C@H](F)C6)nc4c3F)c12 ; C#Cc1c(F)ccc2cc(O)cc(-c3ncc4c(N5CCCCN5C(=O)OC(C)(C)C)nc(OC[C@@]56CCCN5C[C@H](F)C6)nc4c3F)c12 ; C#Cc1c(F)ccc2cc(O)cc(-c3ncc4c(N5CCCN5)nc(OC[C@@]56CCCN5C[C@H](F)C6)nc4c3F)c12 ; C#Cc1c(F)ccc2cc(OC(=O)NC)cc(-c3ncc4c(N5CCCCCN5)nc(OC[C@@]56CCCN5C[C@H](F)C6)nc4c3F)c12 ; C#Cc1c(F)ccc2cc(O)cc(-c3ncc4c(N5CCCCCN5C(=O)OCC)nc(OC[C@@]56CCCN5C[C@H](F)C6)nc4c3F)c12]

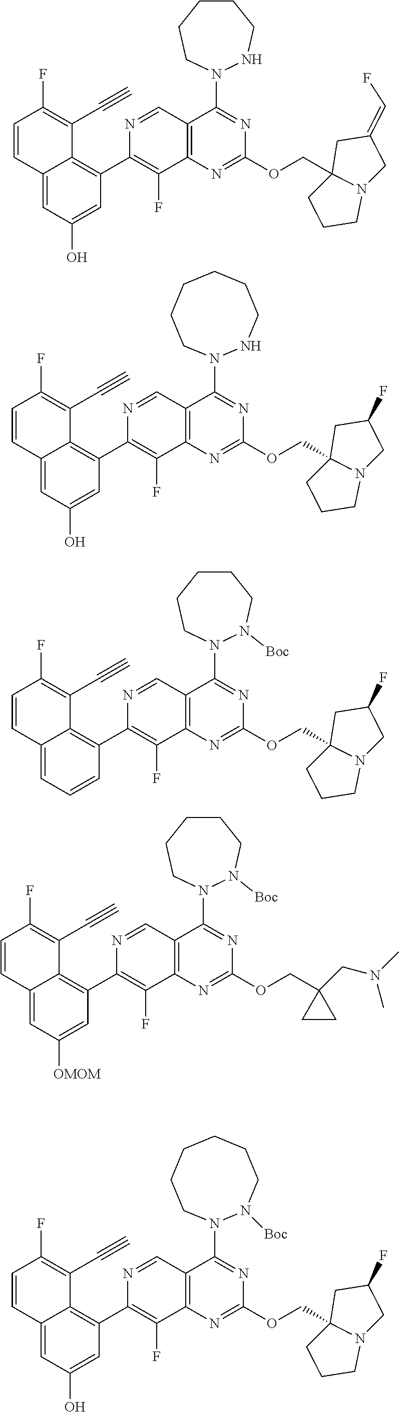

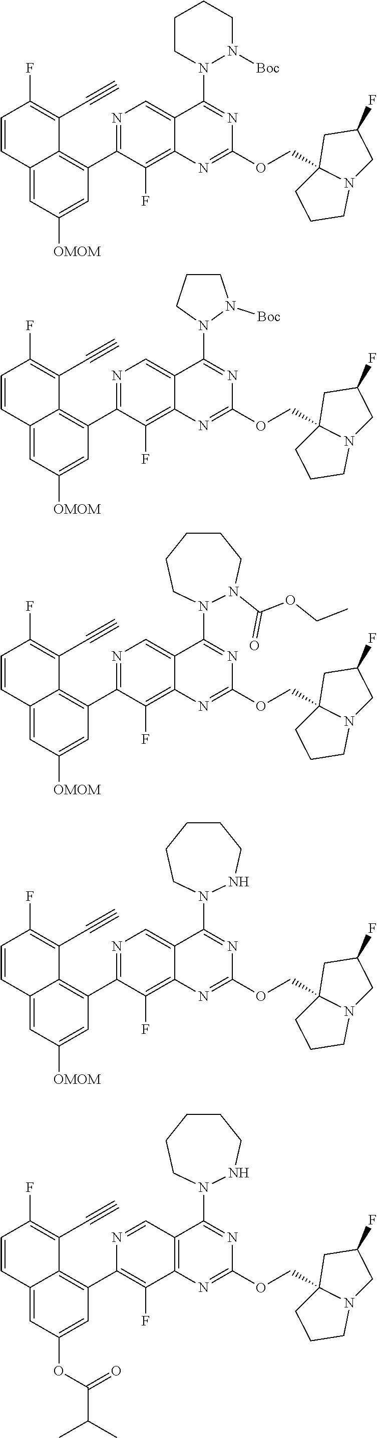

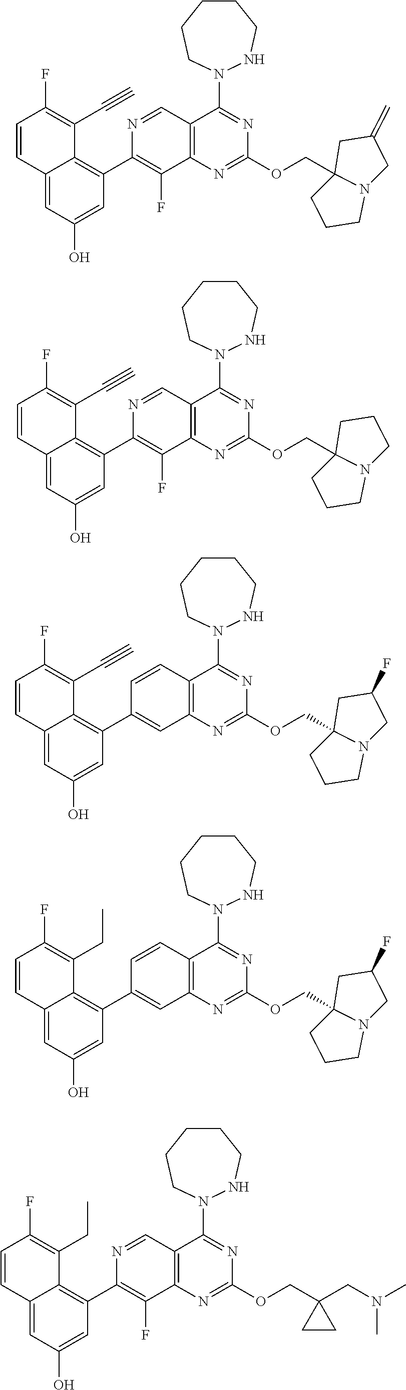 [SMILES: C#Cc1c(F)ccc2cc(O)cc(-c3ncc4c(N5CCCCCN5)nc(OCC56CCCN5CC(=C)C6)nc4c3F)c12 ; C#Cc1c(F)ccc2cc(O)cc(-c3ncc4c(N5CCCCCN5)nc(OCC56CCCN5CCC6)nc4c3F)c12 ; C#Cc1c(F)ccc2cc(O)cc(-c3ccc4c(N5CCCCCN5)nc(OC[C@@]56CCCN5C[C@H](F)C6)nc4c3)c12 ; CCc1c(F)ccc2cc(O)cc(-c3ccc4c(N5CCCCCN5)nc(OC[C@@]56CCCN5C[C@H](F)C6)nc4c3)c12 ; CCc1c(F)ccc2cc(O)cc(-c3ncc4c(N5CCCCCN5)nc(OCC5(CN(C)C)CC5)nc4c3F)c12]

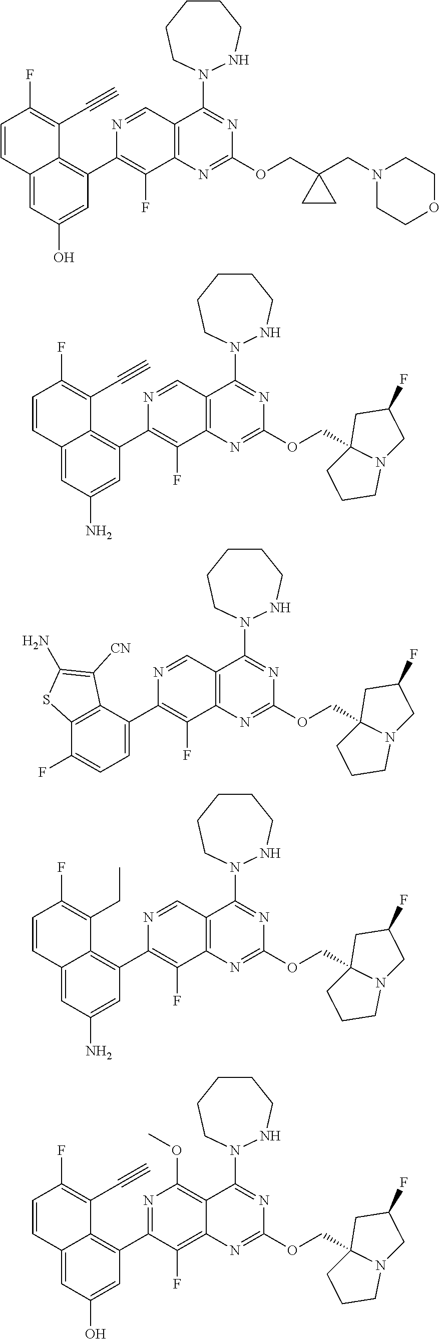 [SMILES: C#Cc1c(F)ccc2cc(O)cc(-c3ncc4c(N5CCCCCN5)nc(OCC5(CN6CCOCC6)CC5)nc4c3F)c12 ; C#Cc1c(F)ccc2cc(N)cc(-c3ncc4c(N5CCCCCN5)nc(OC[C@@]56CCCN5C[C@H](F)C6)nc4c3F)c12 ; N#Cc1c(N)sc2c(F)ccc(-c3ncc4c(N5CCCCCN5)nc(OC[C@@]56CCCN5C[C@H](F)C6)nc4c3F)c12 ; CCc1c(F)ccc2cc(N)cc(-c3ncc4c(N5CCCCCN5)nc(OC[C@@]56CCCN5C[C@H](F)C6)nc4c3F)c12 ; C#Cc1c(F)ccc2cc(O)cc(-c3nc(OC)c4c(N5CCCCCN5)nc(OC[C@@]56CCCN5C[C@H](F)C6)nc4c3F)c12]

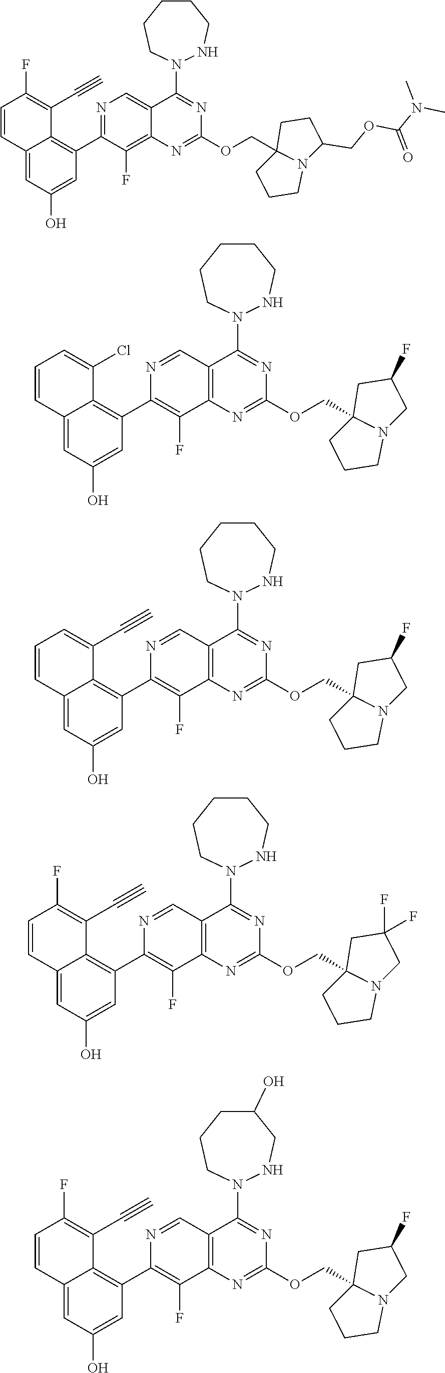 [SMILES: C#Cc1c(F)ccc2cc(O)cc(-c3ncc4c(N5CCCCCN5)nc(OCC56CCCN5C(COC(=O)N(C)C)CC6)nc4c3F)c12 ; Oc1cc(-c2ncc3c(N4CCCCCN4)nc(OC[C@@]45CCCN4C[C@H](F)C5)nc3c2F)c2c(Cl)cccc2c1 ; C#Cc1cccc2cc(O)cc(-c3ncc4c(N5CCCCCN5)nc(OC[C@@]56CCCN5C[C@H](F)C6)nc4c3F)c12 ; C#Cc1c(F)ccc2cc(O)cc(-c3ncc4c(N5CCCCCN5)nc(OC[C@@]56CCCN5CC(F)(F)C6)nc4c3F)c12 ; C#Cc1c(F)ccc2cc(O)cc(-c3ncc4c(N5CCCC(O)CN5)nc(OC[C@@]56CCCN5C[C@H](F)C6)nc4c3F)c12]

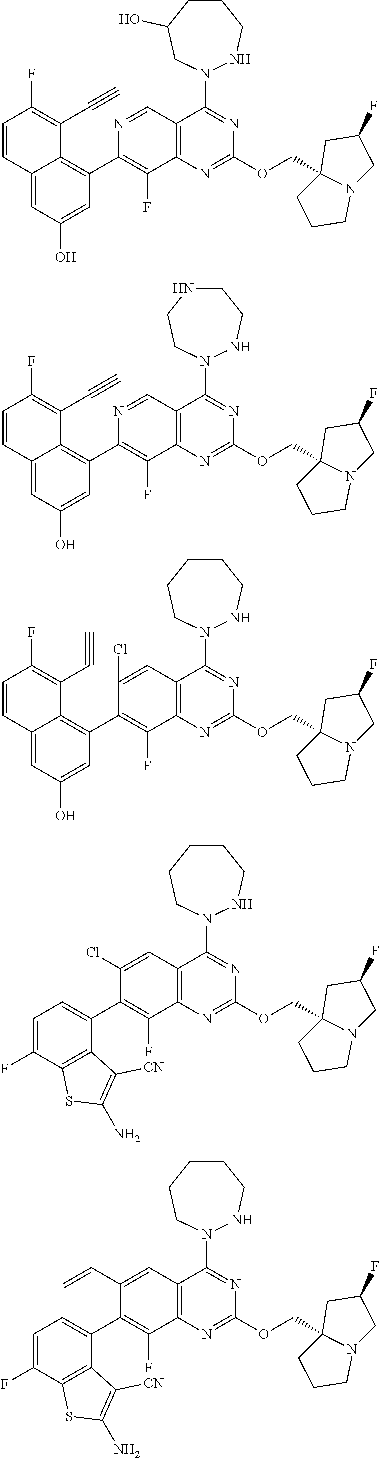 [SMILES: C#Cc1c(F)ccc2cc(O)cc(-c3ncc4c(N5CC(O)CCCN5)nc(OC[C@@]56CCCN5C[C@H](F)C6)nc4c3F)c12 ; C#Cc1c(F)ccc2cc(O)cc(-c3ncc4c(N5CCNCCN5)nc(OC[C@@]56CCCN5C[C@H](F)C6)nc4c3F)c12 ; C#Cc1c(F)ccc2cc(O)cc(-c3c(Cl)cc4c(N5CCCCCN5)nc(OC[C@@]56CCCN5C[C@H](F)C6)nc4c3F)c12 ; N#Cc1c(N)sc2c(F)ccc(-c3c(Cl)cc4c(N5CCCCCN5)nc(OC[C@@]56CCCN5C[C@H](F)C6)nc4c3F)c12 ; C=Cc1cc2c(N3CCCCCN3)nc(OC[C@@]34CCCN3C[C@H](F)C4)nc2c(F)c1-c1ccc(F)c2sc(N)c(C#N)c12]

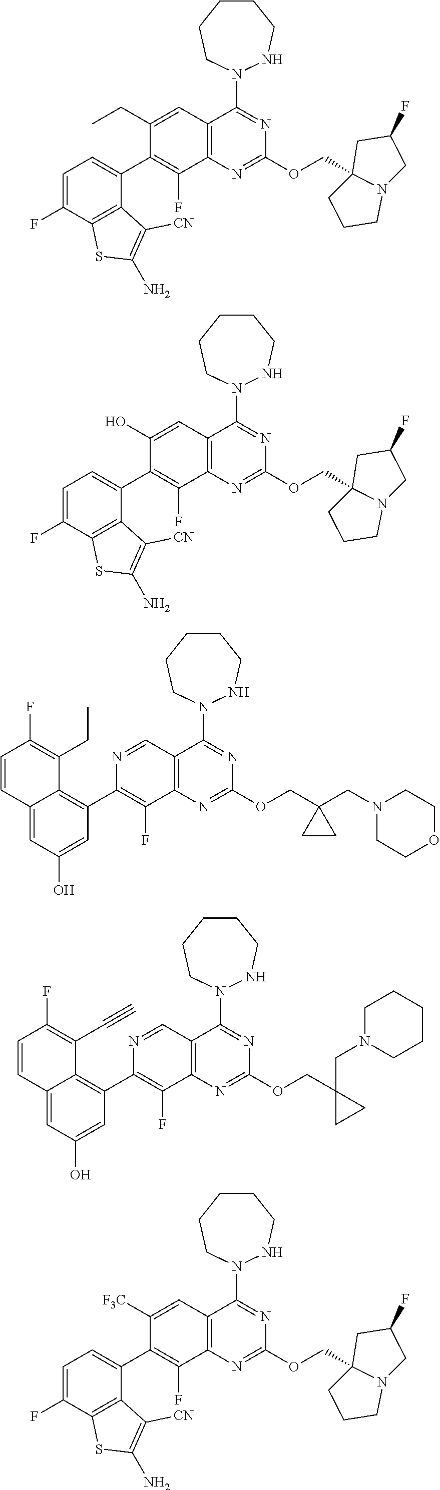 [SMILES: CCc1cc2c(N3CCCCCN3)nc(OC[C@@]34CCCN3C[C@H](F)C4)nc2c(F)c1-c1ccc(F)c2sc(N)c(C#N)c12 ; N#Cc1c(N)sc2c(F)ccc(-c3c(O)cc4c(N5CCCCCN5)nc(OC[C@@]56CCCN5C[C@H](F)C6)nc4c3F)c12 ; CCc1c(F)ccc2cc(O)cc(-c3ncc4c(N5CCCCCN5)nc(OCC5(CN6CCOCC6)CC5)nc4c3F)c12 ; C#Cc1c(F)ccc2cc(O)cc(-c3ncc4c(N5CCCCCN5)nc(OCC5(CN6CCCCC6)CC5)nc4c3F)c12 ; N#Cc1c(N)sc2c(F)ccc(-c3c(C(F)(F)F)cc4c(N5CCCCCN5)nc(OC[C@@]56CCCN5C[C@H](F)C6)nc4c3F)c12]

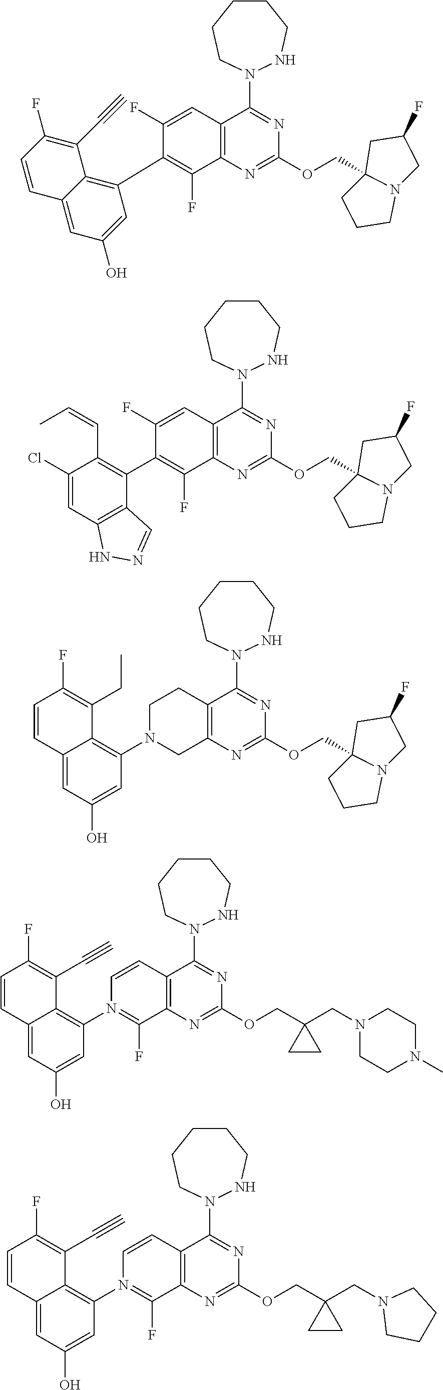

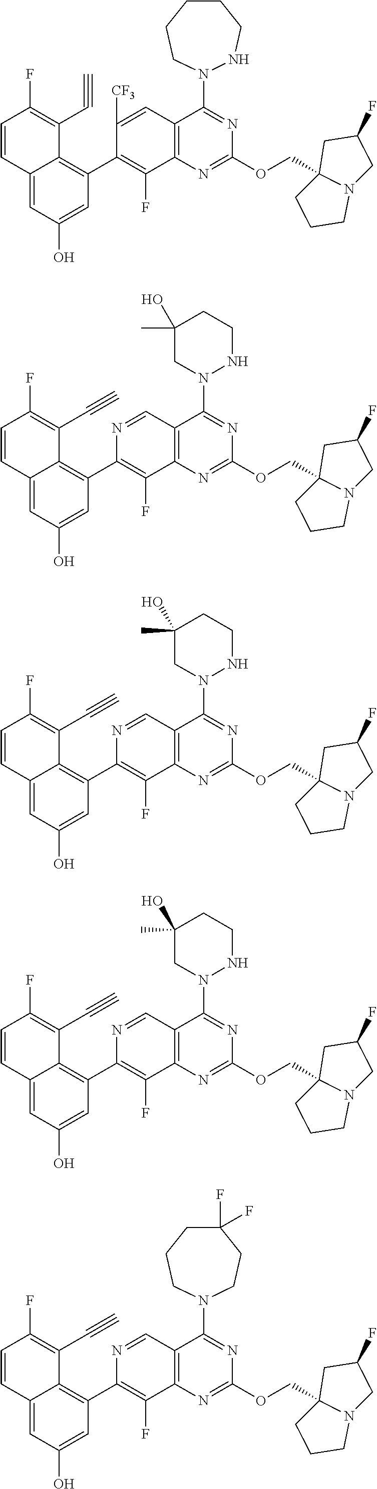 [SMILES: C#Cc1c(F)ccc2cc(O)cc(-c3c(C(F)(F)F)cc4c(N5CCCCCN5)nc(OC[C@@]56CCCN5C[C@H](F)C6)nc4c3F)c12 ; C#Cc1c(F)ccc2cc(O)cc(-c3ncc4c(N5CC(C)(O)CCN5)nc(OC[C@@]56CCCN5C[C@H](F)C6)nc4c3F)c12 ; C#Cc1c(F)ccc2cc(O)cc(-c3ncc4c(N5C[C@](C)(O)CCN5)nc(OC[C@@]56CCCN5C[C@H](F)C6)nc4c3F)c12 ; C#Cc1c(F)ccc2cc(O)cc(-c3ncc4c(N5C[C@@](C)(O)CCN5)nc(OC[C@@]56CCCN5C[C@H](F)C6)nc4c3F)c12 ; C#Cc1c(F)ccc2cc(O)cc(-c3ncc4c(N5CCCC(F)(F)CC5)nc(OC[C@@]56CCCN5C[C@H](F)C6)nc4c3F)c12]

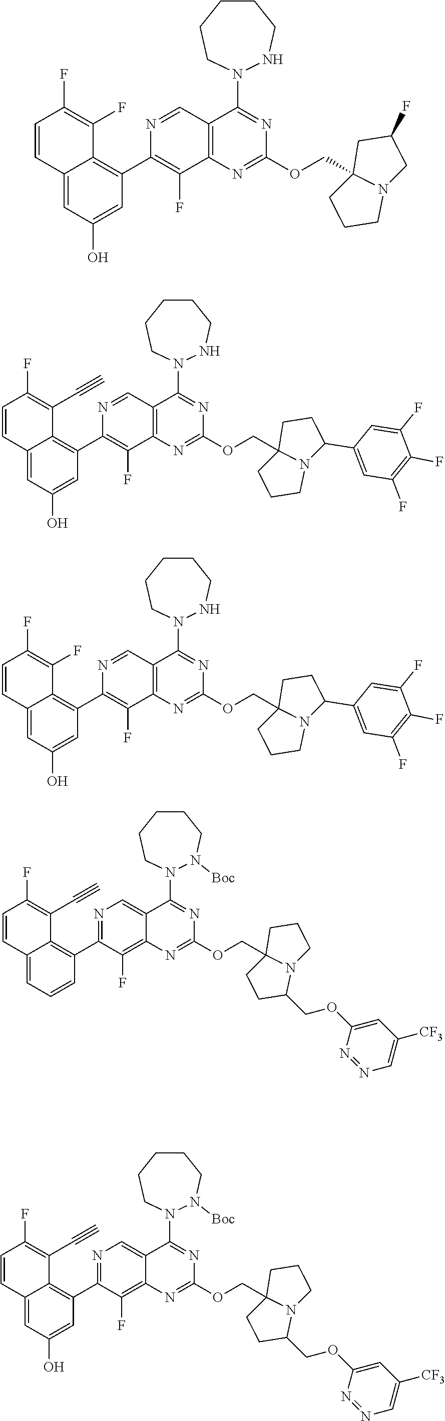 [SMILES: Oc1cc(-c2ncc3c(N4CCCCCN4)nc(OC[C@@]45CCCN4C[C@H](F)C5)nc3c2F)c2c(F)c(F)ccc2c1 ; C#Cc1c(F)ccc2cc(O)cc(-c3ncc4c(N5CCCCCN5)nc(OCC56CCCN5C(c5cc(F)c(F)c(F)c5)CC6)nc4c3F)c12 ; Oc1cc(-c2ncc3c(N4CCCCCN4)nc(OCC45CCCN4C(c4cc(F)c(F)c(F)c4)CC5)nc3c2F)c2c(F)c(F)ccc2c1 ; C#Cc1c(F)ccc2cccc(-c3ncc4c(N5CCCCCN5C(=O)OC(C)(C)C)nc(OCC56CCCN5C(COc5cc(C(F)(F)F)cnn5)CC6)nc4c3F)c12 ; C#Cc1c(F)ccc2cc(O)cc(-c3ncc4c(N5CCCCCN5C(=O)OC(C)(C)C)nc(OCC56CCCN5C(COc5cc(C(F)(F)F)cnn5)CC6)nc4c3F)c12]

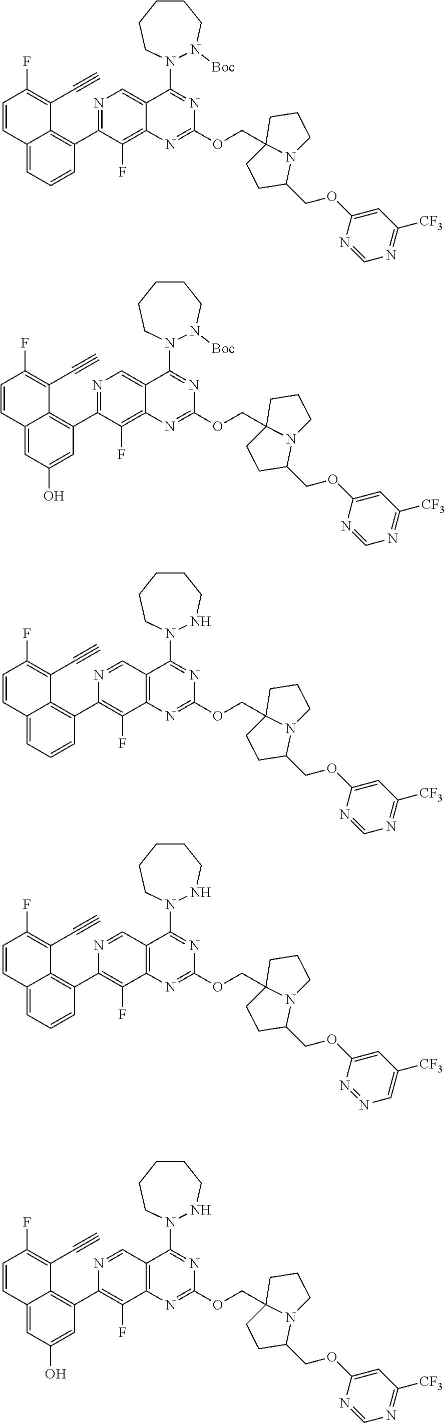 [SMILES: C#Cc1c(F)ccc2cccc(-c3ncc4c(N5CCCCCN5C(=O)OC(C)(C)C)nc(OCC56CCCN5C(COc5cc(C(F)(F)F)ncn5)CC6)nc4c3F)c12 ; C#Cc1c(F)ccc2cc(O)cc(-c3ncc4c(N5CCCCCN5C(=O)OC(C)(C)C)nc(OCC56CCCN5C(COc5cc(C(F)(F)F)ncn5)CC6)nc4c3F)c12 ; C#Cc1c(F)ccc2cccc(-c3ncc4c(N5CCCCCN5)nc(OCC56CCCN5C(COc5cc(C(F)(F)F)ncn5)CC6)nc4c3F)c12 ; C#Cc1c(F)ccc2cccc(-c3ncc4c(N5CCCCCN5)nc(OCC56CCCN5C(COc5cc(C(F)(F)F)cnn5)CC6)nc4c3F)c12 ; C#Cc1c(F)ccc2cc(O)cc(-c3ncc4c(N5CCCCCN5)nc(OCC56CCCN5C(COc5cc(C(F)(F)F)ncn5)CC6)nc4c3F)c12]

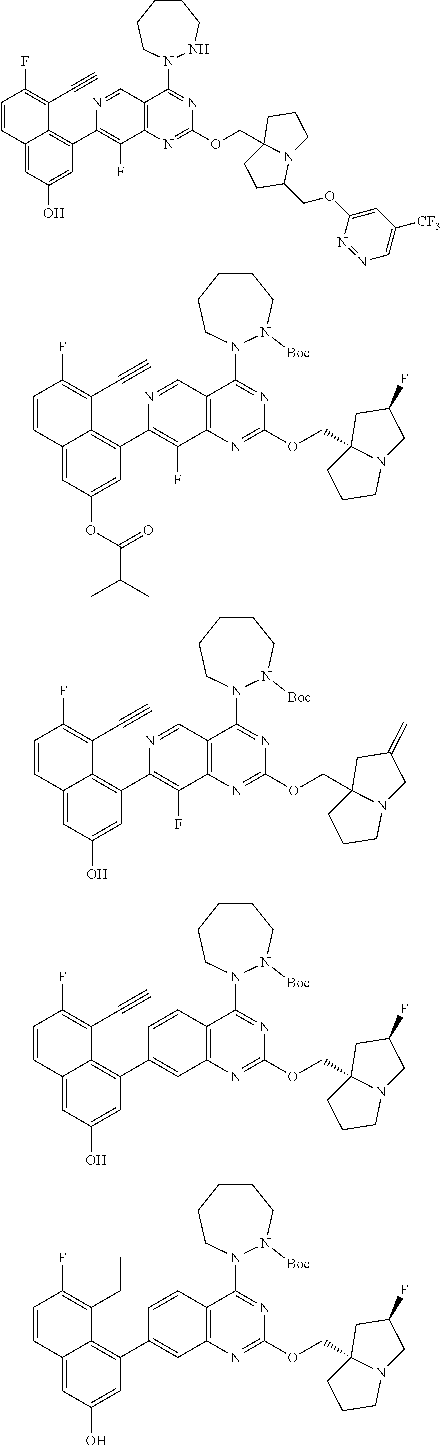 [SMILES: C#Cc1c(F)ccc2cc(O)cc(-c3ncc4c(N5CCCCCN5)nc(OCC56CCCN5C(COc5cc(C(F)(F)F)cnn5)CC6)nc4c3F)c12 ; C#Cc1c(F)ccc2cc(OC(=O)C(C)C)cc(-c3ncc4c(N5CCCCCN5C(=O)OC(C)(C)C)nc(OC[C@@]56CCCN5C[C@H](F)C6)nc4c3F)c12 ; C#Cc1c(F)ccc2cc(O)cc(-c3ncc4c(N5CCCCCN5C(=O)OC(C)(C)C)nc(OCC56CCCN5CC(=C)C6)nc4c3F)c12 ; C#Cc1c(F)ccc2cc(O)cc(-c3ccc4c(N5CCCCCN5C(=O)OC(C)(C)C)nc(OC[C@@]56CCCN5C[C@H](F)C6)nc4c3)c12 ; CCc1c(F)ccc2cc(O)cc(-c3ccc4c(N5CCCCCN5C(=O)OC(C)(C)C)nc(OC[C@@]56CCCN5C[C@H](F)C6)nc4c3)c12]

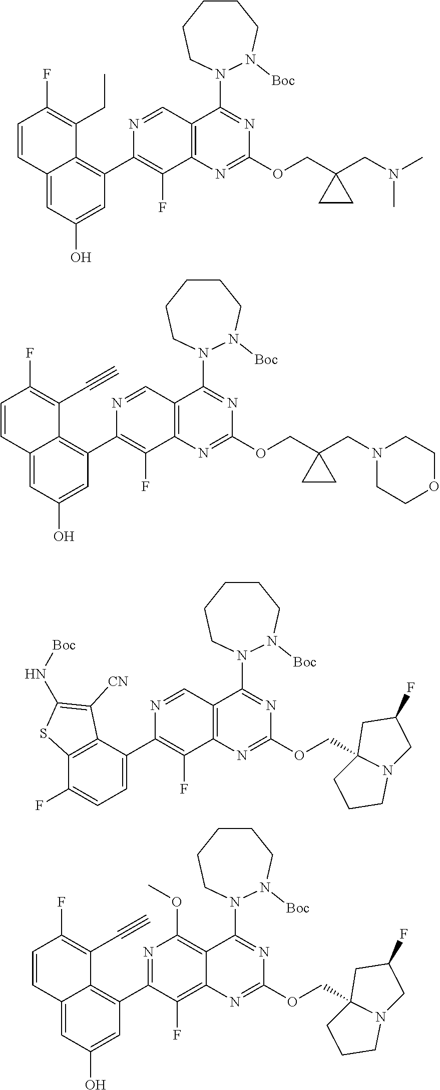 [SMILES: CCc1c(F)ccc2cc(O)cc(-c3ncc4c(N5CCCCCN5C(=O)OC(C)(C)C)nc(OCC5(CN(C)C)CC5)nc4c3F)c12 ; C#Cc1c(F)ccc2cc(O)cc(-c3ncc4c(N5CCCCCN5C(=O)OC(C)(C)C)nc(OCC5(CN6CCOCC6)CC5)nc4c3F)c12 ; CC(C)(C)OC(=O)Nc1sc2c(F)ccc(-c3ncc4c(N5CCCCCN5C(=O)OC(C)(C)C)nc(OC[C@@]56CCCN5C[C@H](F)C6)nc4c3F)c2c1C#N ; C#Cc1c(F)ccc2cc(O)cc(-c3nc(OC)c4c(N5CCCCCN5C(=O)OC(C)(C)C)nc(OC[C@@]56CCCN5C[C@H](F)C6)nc4c3F)c12]

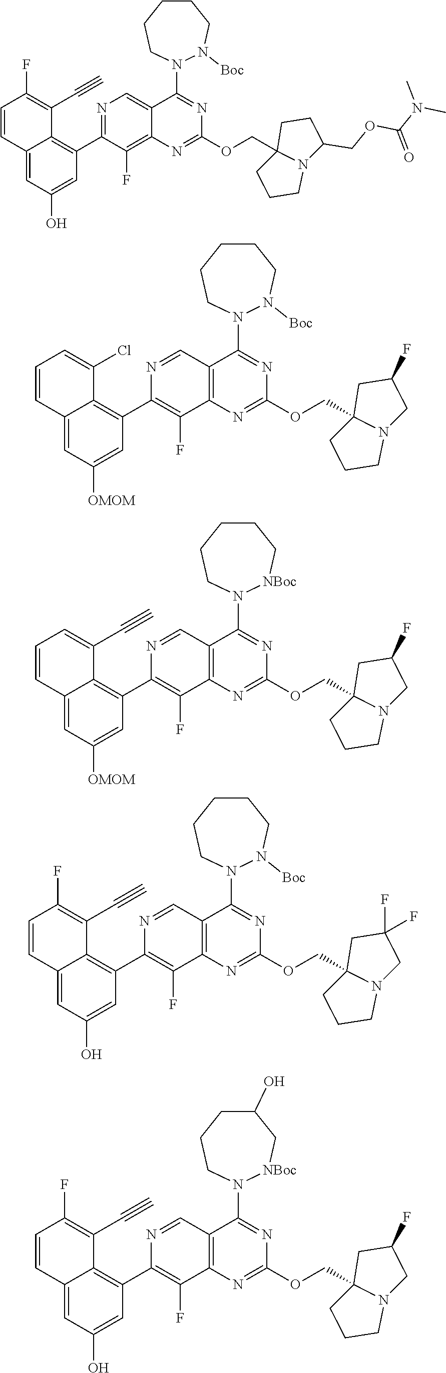

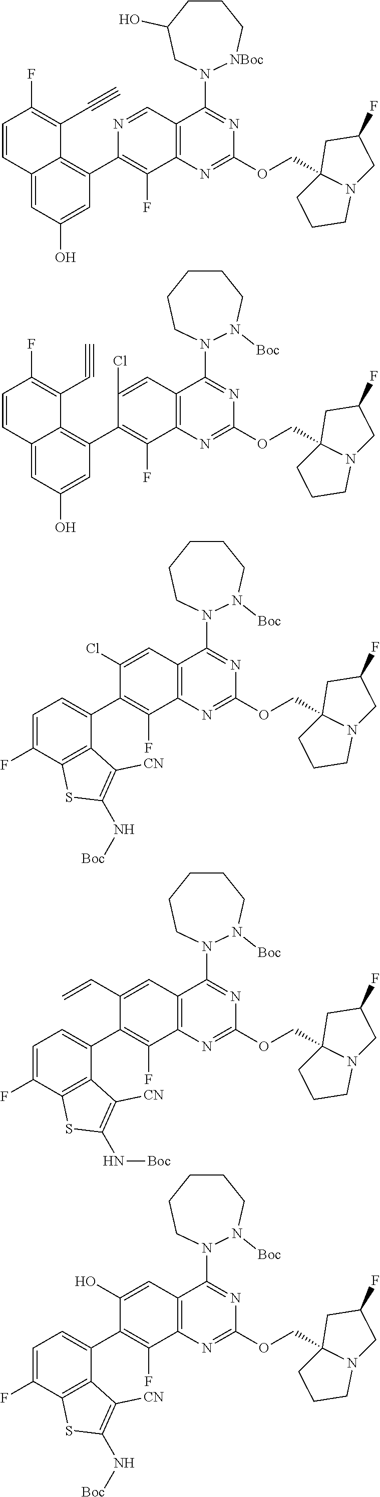

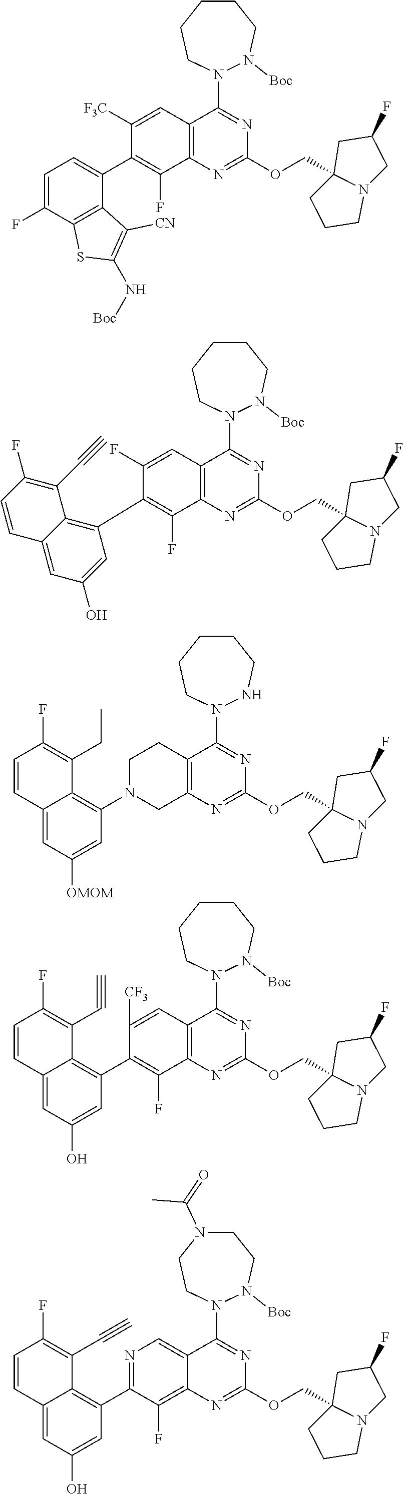

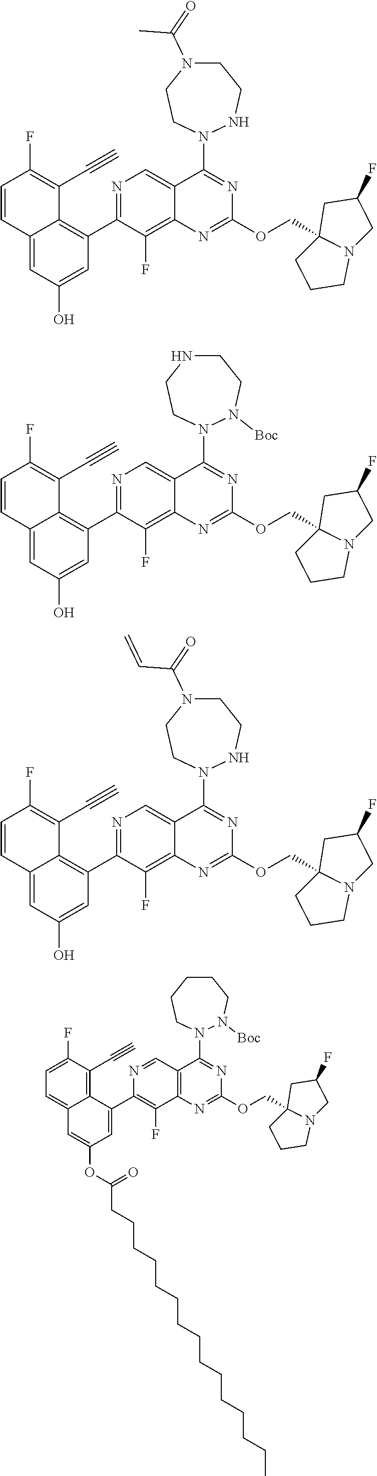 [SMILES: C#Cc1c(F)ccc2cc(O)cc(-c3ncc4c(N5CCN(C(C)=O)CCN5)nc(OC[C@@]56CCCN5C[C@H](F)C6)nc4c3F)c12 ; C#Cc1c(F)ccc2cc(O)cc(-c3ncc4c(N5CCNCCN5C(=O)OC(C)(C)C)nc(OC[C@@]56CCCN5C[C@H](F)C6)nc4c3F)c12 ; C#Cc1c(F)ccc2cc(O)cc(-c3ncc4c(N5CCN(C(=O)C=C)CCN5)nc(OC[C@@]56CCCN5C[C@H](F)C6)nc4c3F)c12 ; C#Cc1c(F)ccc2cc(OC(=O)CCCCCCCCCCCCCCC)cc(-c3ncc4c(N5CCCCCN5C(=O)OC(C)(C)C)nc(OC[C@@]56CCCN5C[C@H](F)C6)nc4c3F)c12]

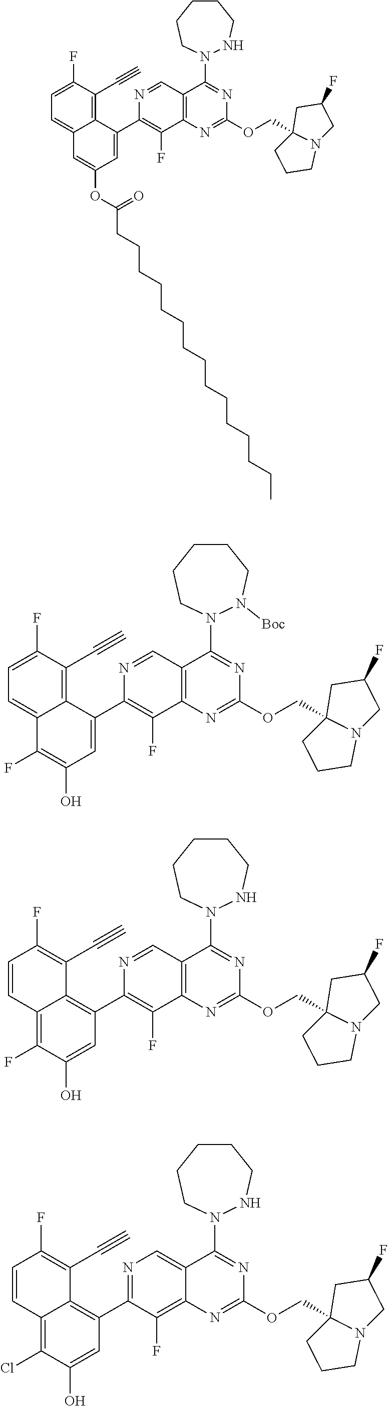 [SMILES: C#Cc1c(F)ccc2cc(OC(=O)CCCCCCCCCCCCCCC)cc(-c3ncc4c(N5CCCCCN5)nc(OC[C@@]56CCCN5C[C@H](F)C6)nc4c3F)c12 ; C#Cc1c(F)ccc2c(F)c(O)cc(-c3ncc4c(N5CCCCCN5C(=O)OC(C)(C)C)nc(OC[C@@]56CCCN5C[C@H](F)C6)nc4c3F)c12 ; C#Cc1c(F)ccc2c(F)c(O)cc(-c3ncc4c(N5CCCCCN5)nc(OC[C@@]56CCCN5C[C@H](F)C6)nc4c3F)c12 ; C#Cc1c(F)ccc2c(Cl)c(O)cc(-c3ncc4c(N5CCCCCN5)nc(OC[C@@]56CCCN5C[C@H](F)C6)nc4c3F)c12]

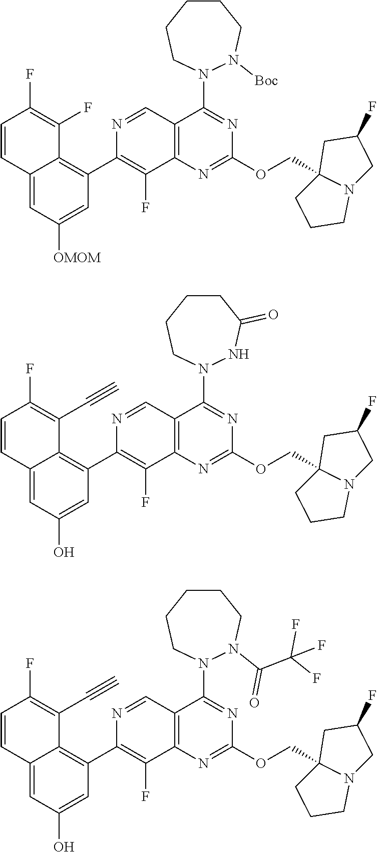

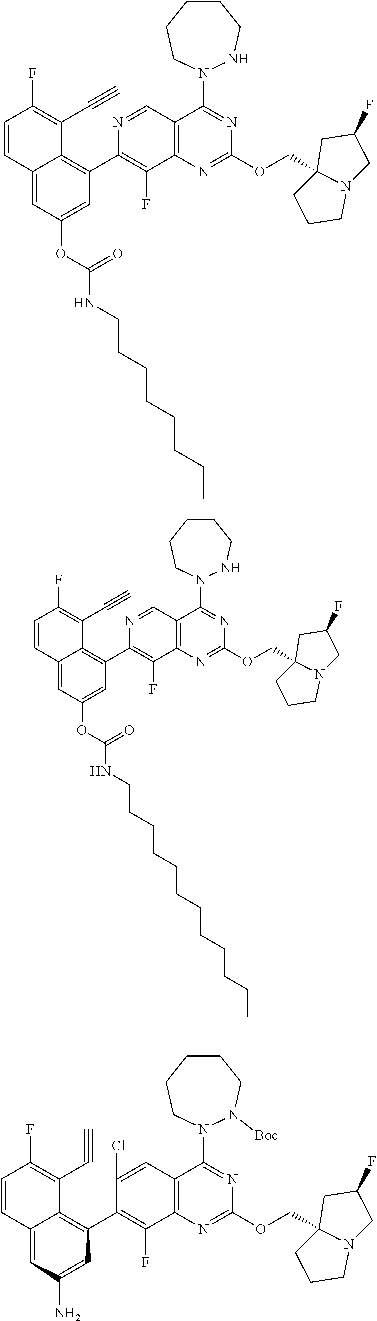

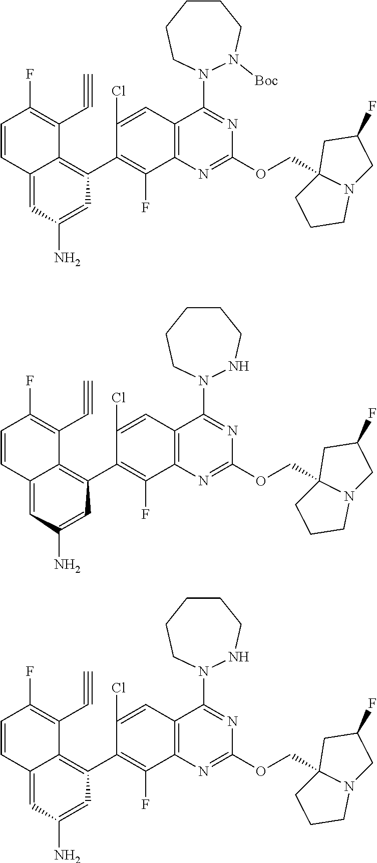

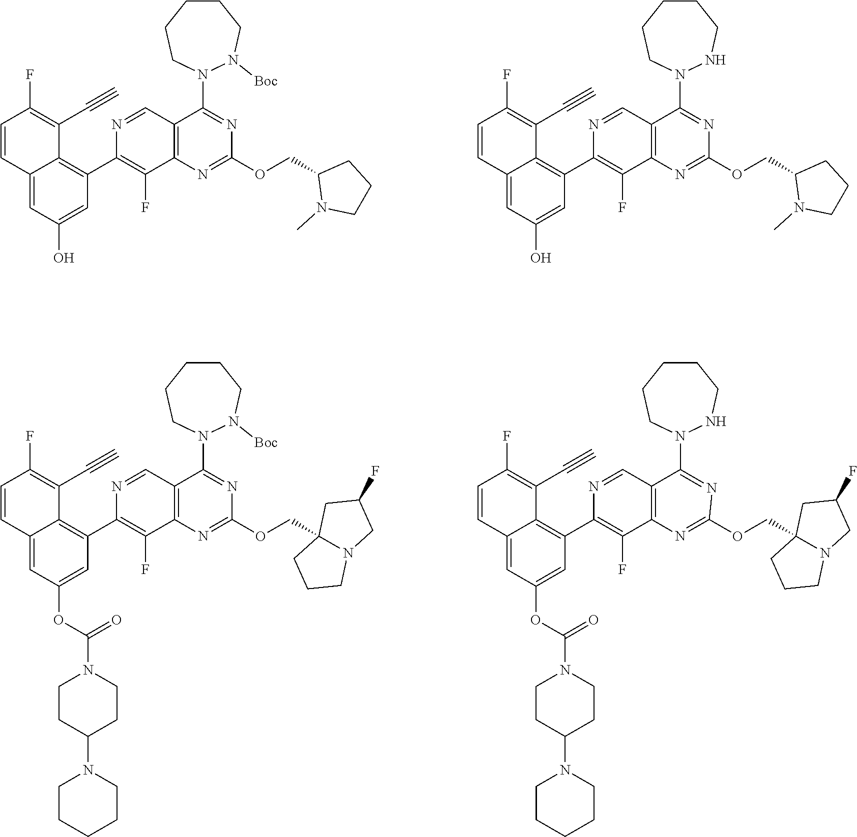 [SMILES: C#Cc1c(F)ccc2cc(O)cc(-c3ncc4c(N5CCCCCN5C(=O)OC(C)(C)C)nc(OC[C@@H]5CCCN5C)nc4c3F)c12 ; C#Cc1c(F)ccc2cc(O)cc(-c3ncc4c(N5CCCCCN5)nc(OC[C@@H]5CCCN5C)nc4c3F)c12 ; C#Cc1c(F)ccc2cc(OC(=O)N3CCC(N4CCCCC4)CC3)cc(-c3ncc4c(N5CCCCCN5C(=O)OC(C)(C)C)nc(OC[C@@]56CCCN5C[C@H](F)C6)nc4c3F)c12 ; C#Cc1c(F)ccc2cc(OC(=O)N3CCC(N4CCCCC4)CC3)cc(-c3ncc4c(N5CCCCCN5)nc(OC[C@@]56CCCN5C[C@H](F)C6)nc4c3F)c12]

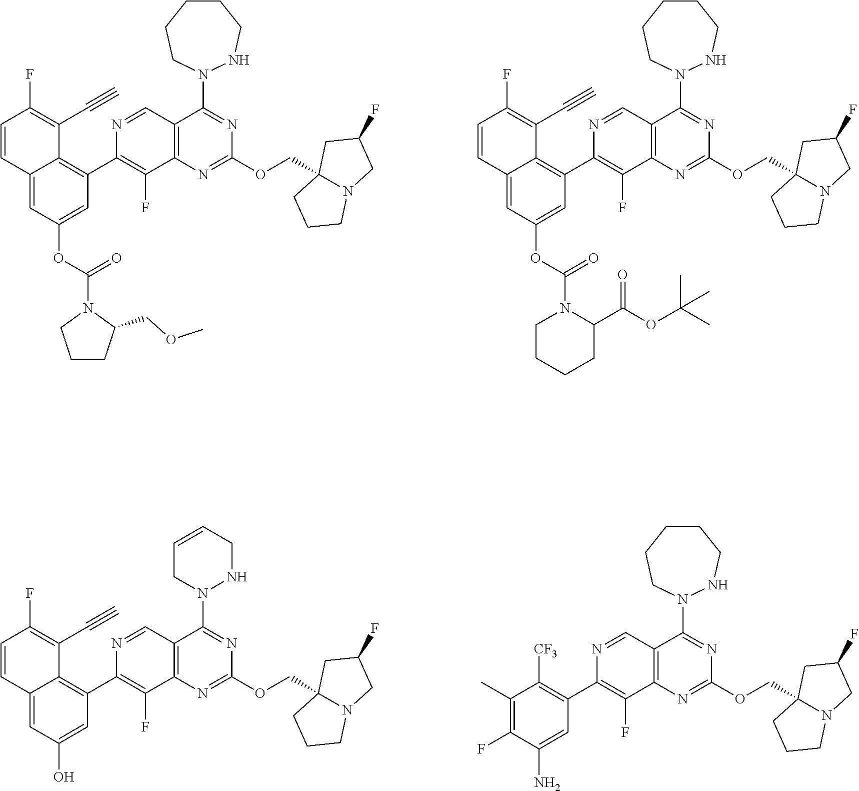 [SMILES: C#Cc1c(F)ccc2cc(OC(=O)N3CCC[C@H]3COC)cc(-c3ncc4c(N5CCCCCN5)nc(OC[C@@]56CCCN5C[C@H](F)C6)nc4c3F)c12 ; C#Cc1c(F)ccc2cc(OC(=O)N3CCCCC3C(=O)OC(C)(C)C)cc(-c3ncc4c(N5CCCCCN5)nc(OC[C@@]56CCCN5C[C@H](F)C6)nc4c3F)c12 ; C#Cc1c(F)ccc2cc(O)cc(-c3ncc4c(N5CC=CCN5)nc(OC[C@@]56CCCN5C[C@H](F)C6)nc4c3F)c12 ; Cc1c(F)c(N)cc(-c2ncc3c(N4CCCCCN4)nc(OC[C@@]45CCCN4C[C@H](F)C5)nc3c2F)c1C(F)(F)F]

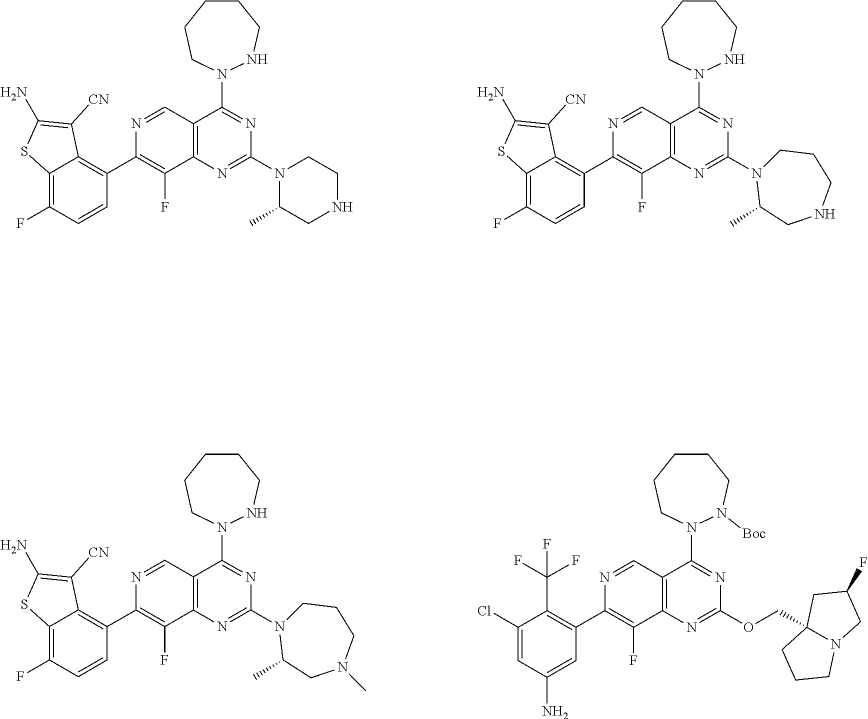 [SMILES: C[C@H]1CNCCN1c1nc(N2CCCCCN2)c2cnc(-c3ccc(F)c4sc(N)c(C#N)c34)c(F)c2n1 ; C[C@H]1CNCCCN1c1nc(N2CCCCCN2)c2cnc(-c3ccc(F)c4sc(N)c(C#N)c34)c(F)c2n1 ; C[C@H]1CN(C)CCCN1c1nc(N2CCCCCN2)c2cnc(-c3ccc(F)c4sc(N)c(C#N)c34)c(F)c2n1 ; CC(C)(C)OC(=O)N1CCCCCN1c1nc(OC[C@@]23CCCN2C[C@H](F)C3)nc2c(F)c(-c3cc(N)cc(Cl)c3C(F)(F)F)ncc12]

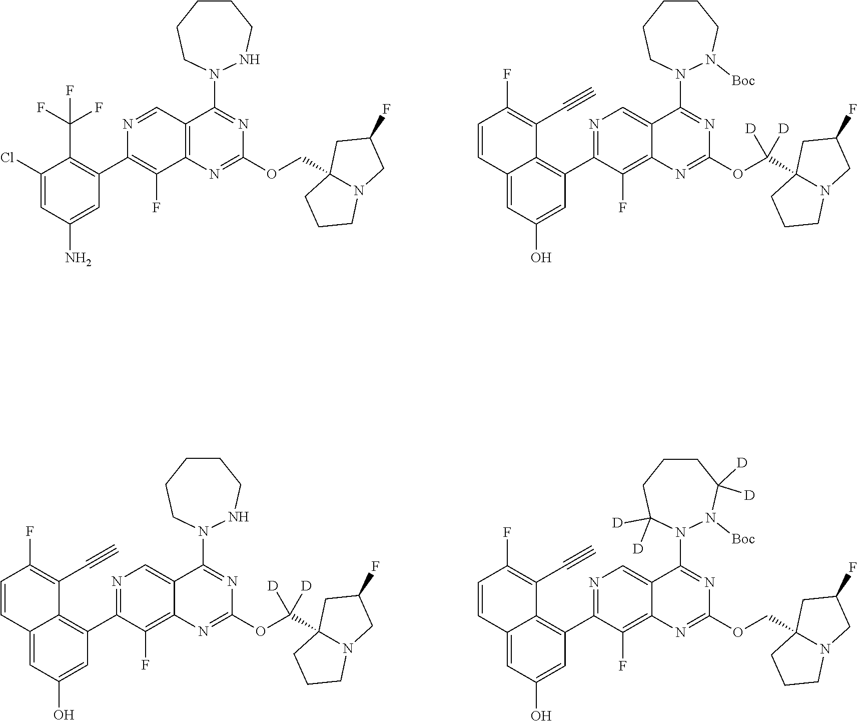 [SMILES: Nc1cc(Cl)c(C(F)(F)F)c(-c2ncc3c(N4CCCCCN4)nc(OC[C@@]45CCCN4C[C@H](F)C5)nc3c2F)c1 ; [2H]C([2H])(Oc1nc(N2CCCCCN2C(=O)OC(C)(C)C)c2cnc(-c3cc(O)cc4ccc(F)c(C#C)c34)c(F)c2n1)[C@@]12CCCN1C[C@H](F)C2 ; [2H]C([2H])(Oc1nc(N2CCCCCN2)c2cnc(-c3cc(O)cc4ccc(F)c(C#C)c34)c(F)c2n1)[C@@]12CCCN1C[C@H](F)C2 ; [2H]C1([2H])CCCC([2H])([2H])N(c2nc(OC[C@@]34CCCN3C[C@H](F)C4)nc3c(F)c(-c4cc(O)cc5ccc(F)c(C#C)c45)ncc23)N1C(=O)OC(C)(C)C]

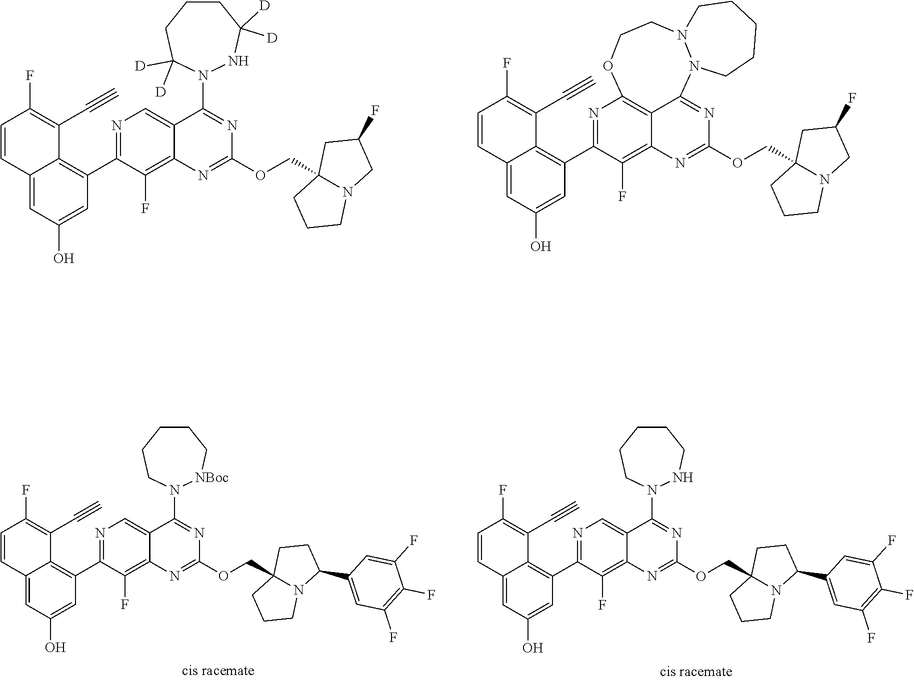 [SMILES: [2H]C1([2H])CCCC([2H])([2H])N(c2nc(OC[C@@]34CCCN3C[C@H](F)C4)nc3c(F)c(-c4cc(O)cc5ccc(F)c(C#C)c45)ncc23)N1 ; C#Cc1c(F)ccc2cc(O)cc(-c3nc4c5c(nc(OC[C@@]67CCCN6C[C@H](F)C7)nc5c3F)N3CCCCCN3CCO4)c12 ; C#Cc1c(F)ccc2cc(O)cc(-c3ncc4c(N5CCCCCN5C(=O)OC(C)(C)C)nc(OC[C@]56CCCN5[C@H](c5cc(F)c(F)c(F)c5)CC6)nc4c3F)c12 ; C#Cc1c(F)ccc2cc(O)cc(-c3ncc4c(N5CCCCCN5)nc(OC[C@]56CCCN5[C@H](c5cc(F)c(F)c(F)c5)CC6)nc4c3F)c12]

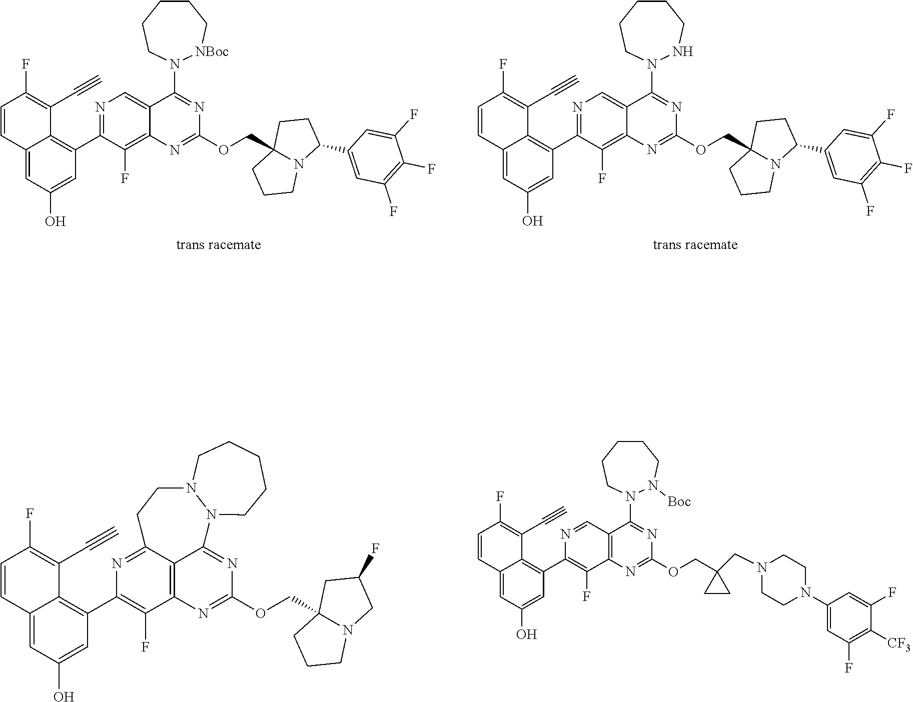 [SMILES: C#Cc1c(F)ccc2cc(O)cc(-c3ncc4c(N5CCCCCN5C(=O)OC(C)(C)C)nc(OC[C@]56CCCN5[C@@H](c5cc(F)c(F)c(F)c5)CC6)nc4c3F)c12 ; C#Cc1c(F)ccc2cc(O)cc(-c3ncc4c(N5CCCCCN5)nc(OC[C@]56CCCN5[C@@H](c5cc(F)c(F)c(F)c5)CC6)nc4c3F)c12 ; C#Cc1c(F)ccc2cc(O)cc(-c3nc4c5c(nc(OC[C@@]67CCCN6C[C@H](F)C7)nc5c3F)N3CCCCCN3CC4)c12 ; C#Cc1c(F)ccc2cc(O)cc(-c3ncc4c(N5CCCCCN5C(=O)OC(C)(C)C)nc(OCC5(CN6CCN(c7cc(F)c(C(F)(F)F)c(F)c7)CC6)CC5)nc4c3F)c12]

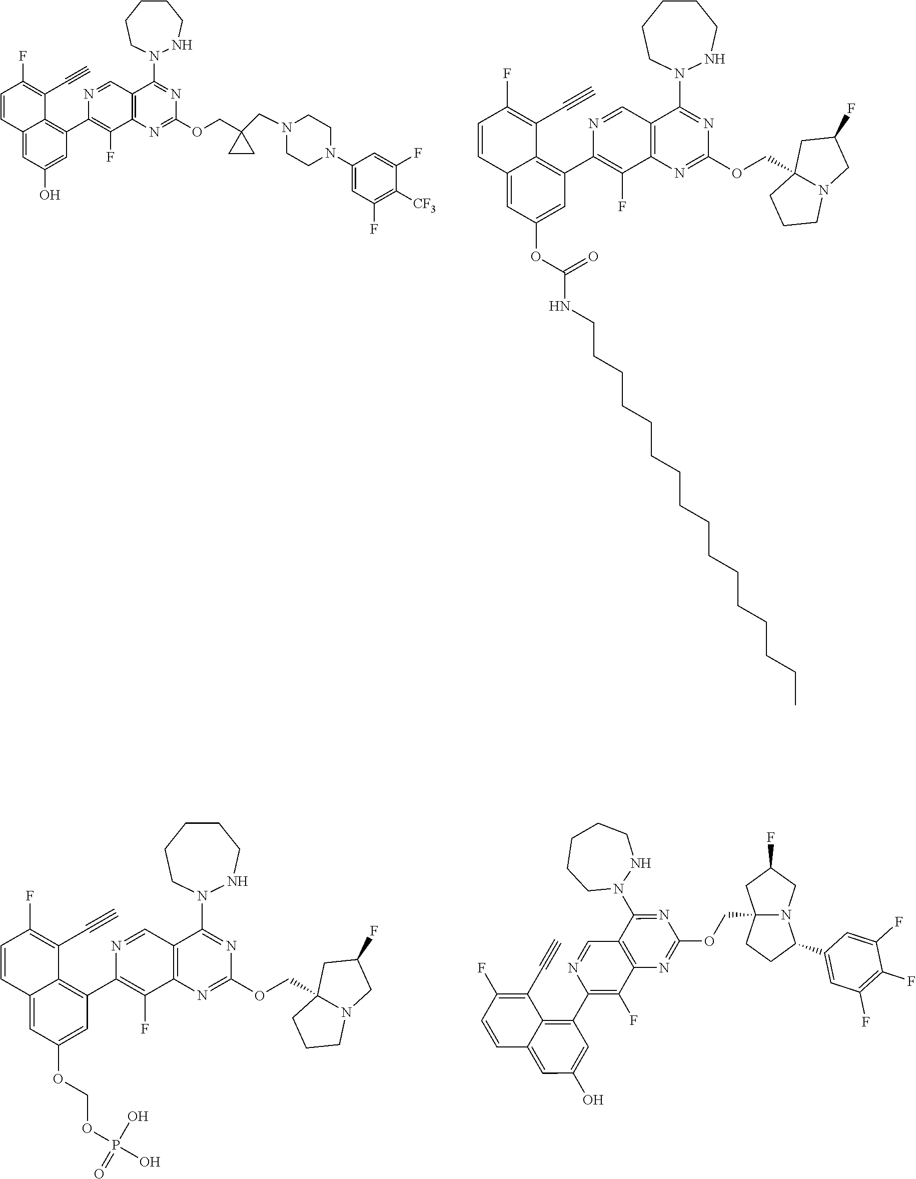 [SMILES: C#Cc1c(F)ccc2cc(O)cc(-c3ncc4c(N5CCCCCN5)nc(OCC5(CN6CCN(c7cc(F)c(C(F)(F)F)c(F)c7)CC6)CC5)nc4c3F)c12 ; C#Cc1c(F)ccc2cc(OC(=O)NCCCCCCCCCCCCCCCC)cc(-c3ncc4c(N5CCCCCN5)nc(OC[C@@]56CCCN5C[C@H](F)C6)nc4c3F)c12 ; C#Cc1c(F)ccc2cc(OCOP(=O)(O)O)cc(-c3ncc4c(N5CCCCCN5)nc(OC[C@@]56CCCN5C[C@H](F)C6)nc4c3F)c12 ; C#Cc1c(F)ccc2cc(O)cc(-c3ncc4c(N5CCCCCN5)nc(OC[C@@]56CC[C@@H](c7cc(F)c(F)c(F)c7)N5C[C@H](F)C6)nc4c3F)c12]

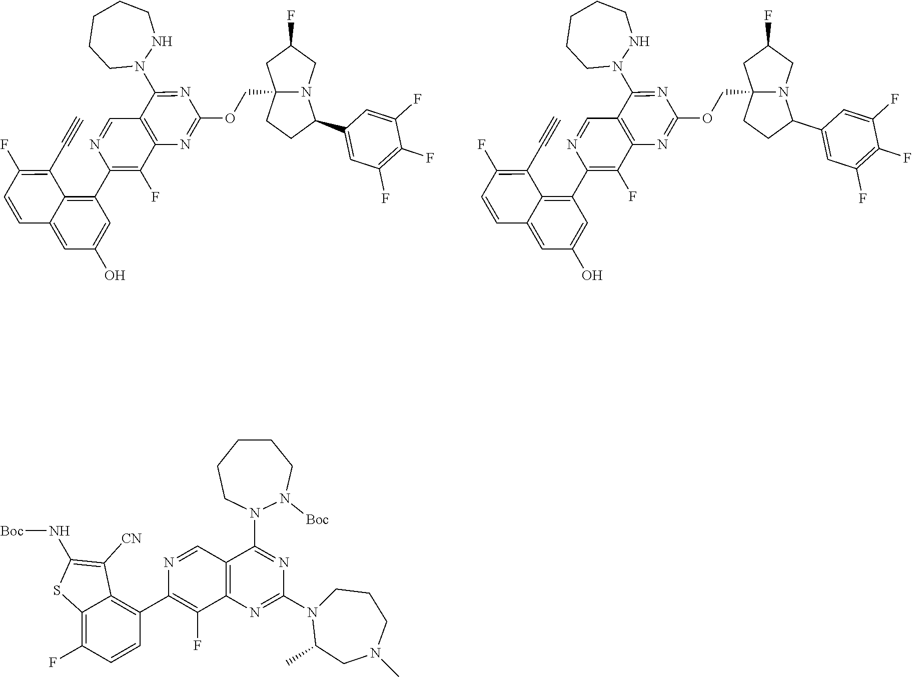 [SMILES: C#Cc1c(F)ccc2cc(O)cc(-c3ncc4c(N5CCCCCN5)nc(OC[C@@]56CC[C@H](c7cc(F)c(F)c(F)c7)N5C[C@H](F)C6)nc4c3F)c12 ; C#Cc1c(F)ccc2cc(O)cc(-c3ncc4c(N5CCCCCN5)nc(OC[C@@]56CCC(c7cc(F)c(F)c(F)c7)N5C[C@H](F)C6)nc4c3F)c12 ; C[C@H]1CN(C)CCCN1c1nc(N2CCCCCN2C(=O)OC(C)(C)C)c2cnc(-c3ccc(F)c4sc(NC(=O)OC(C)(C)C)c(C#N)c34)c(F)c2n1]

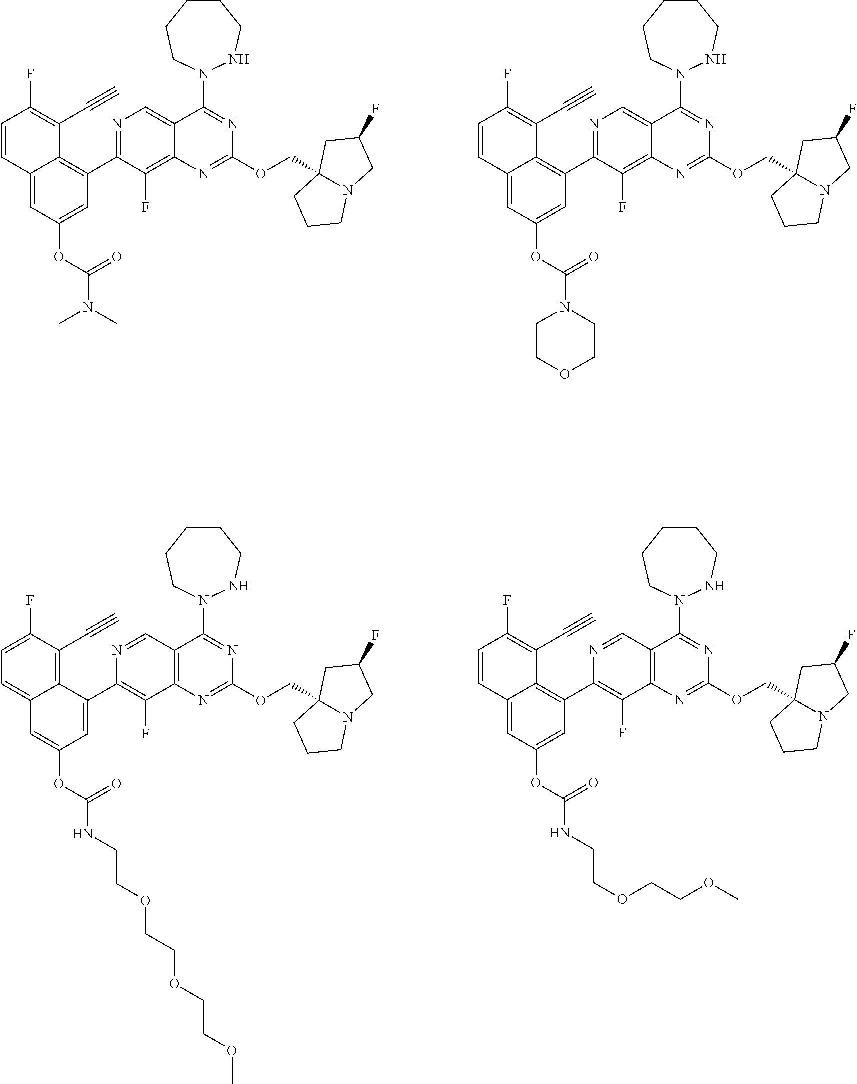 [SMILES: C#Cc1c(F)ccc2cc(OC(=O)N(C)C)cc(-c3ncc4c(N5CCCCCN5)nc(OC[C@@]56CCCN5C[C@H](F)C6)nc4c3F)c12 ; C#Cc1c(F)ccc2cc(OC(=O)N3CCOCC3)cc(-c3ncc4c(N5CCCCCN5)nc(OC[C@@]56CCCN5C[C@H](F)C6)nc4c3F)c12 ; C#Cc1c(F)ccc2cc(OC(=O)NCCOCCOCCOC)cc(-c3ncc4c(N5CCCCCN5)nc(OC[C@@]56CCCN5C[C@H](F)C6)nc4c3F)c12 ; C#Cc1c(F)ccc2cc(OC(=O)NCCOCCOC)cc(-c3ncc4c(N5CCCCCN5)nc(OC[C@@]56CCCN5C[C@H](F)C6)nc4c3F)c12]

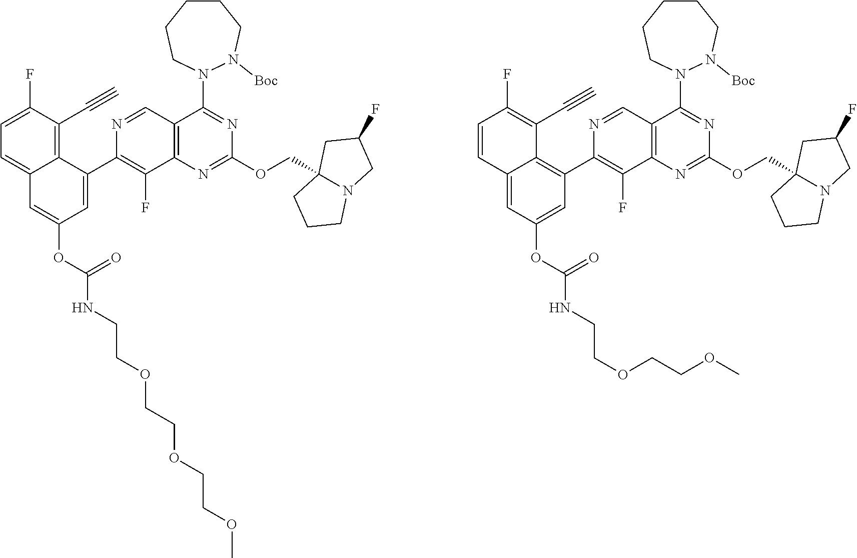 [SMILES: C#Cc1c(F)ccc2cc(OC(=O)NCCOCCOCCOC)cc(-c3ncc4c(N5CCCCCN5C(=O)OC(C)(C)C)nc(OC[C@@]56CCCN5C[C@H](F)C6)nc4c3F)c12 ; C#Cc1c(F)ccc2cc(OC(=O)NCCOCCOC)cc(-c3ncc4c(N5CCCCCN5C(=O)OC(C)(C)C)nc(OC[C@@]56CCCN5C[C@H](F)C6)nc4c3F)c12]

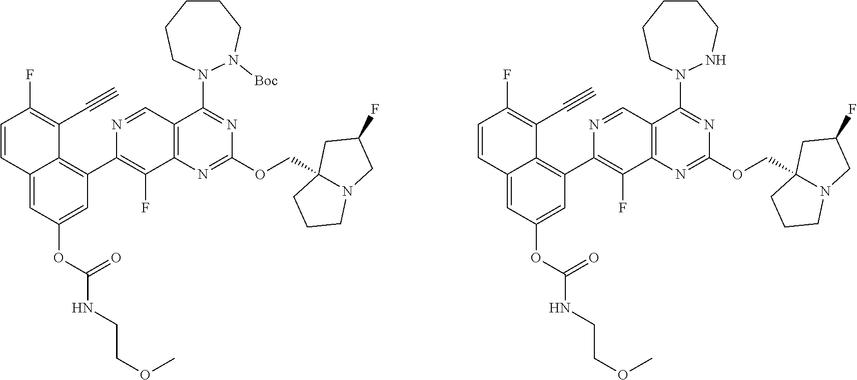 [SMILES: C#Cc1c(F)ccc2cc(OC(=O)NCCOC)cc(-c3ncc4c(N5CCCCCN5C(=O)OC(C)(C)C)nc(OC[C@@]56CCCN5C[C@H](F)C6)nc4c3F)c12 ; C#Cc1c(F)ccc2cc(OC(=O)NCCOC)cc(-c3ncc4c(N5CCCCCN5)nc(OC[C@@]56CCCN5C[C@H](F)C6)nc4c3F)c12]

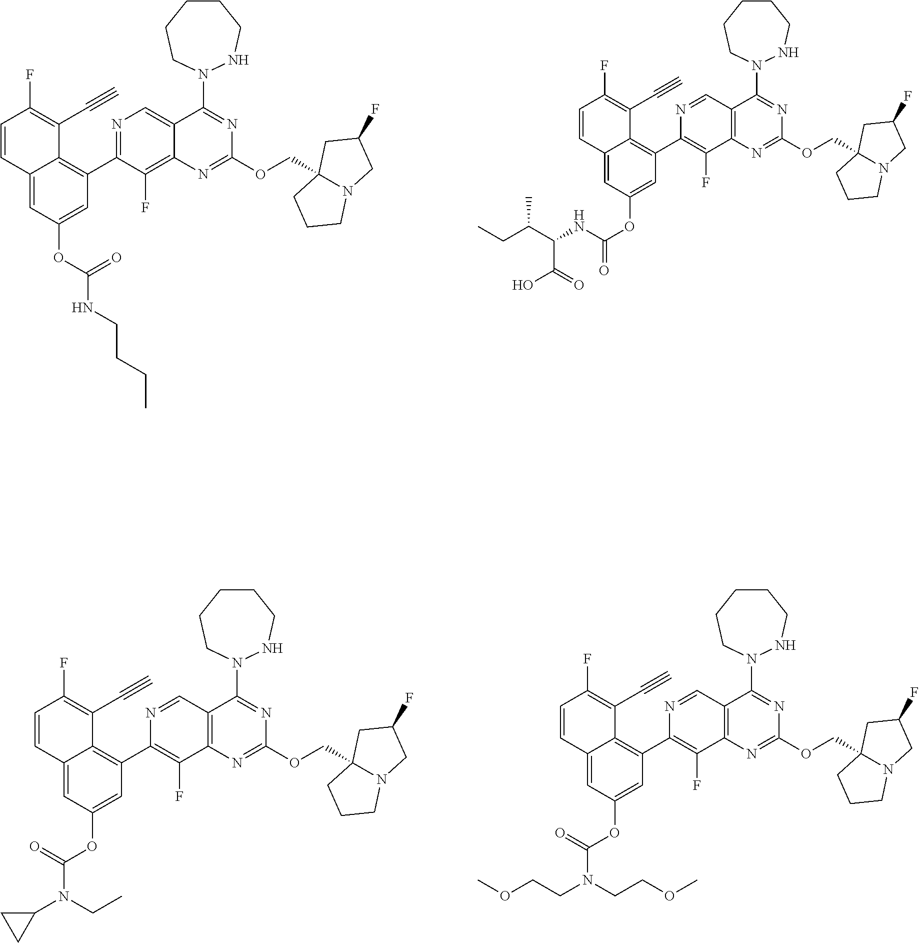 [SMILES: C#Cc1c(F)ccc2cc(OC(=O)NCCCC)cc(-c3ncc4c(N5CCCCCN5)nc(OC[C@@]56CCCN5C[C@H](F)C6)nc4c3F)c12 ; C#Cc1c(F)ccc2cc(OC(=O)N[C@H](C(=O)O)[C@@H](C)CC)cc(-c3ncc4c(N5CCCCCN5)nc(OC[C@@]56CCCN5C[C@H](F)C6)nc4c3F)c12 ; C#Cc1c(F)ccc2cc(OC(=O)N(CC)C3CC3)cc(-c3ncc4c(N5CCCCCN5)nc(OC[C@@]56CCCN5C[C@H](F)C6)nc4c3F)c12 ; C#Cc1c(F)ccc2cc(OC(=O)N(CCOC)CCOC)cc(-c3ncc4c(N5CCCCCN5)nc(OC[C@@]56CCCN5C[C@H](F)C6)nc4c3F)c12]

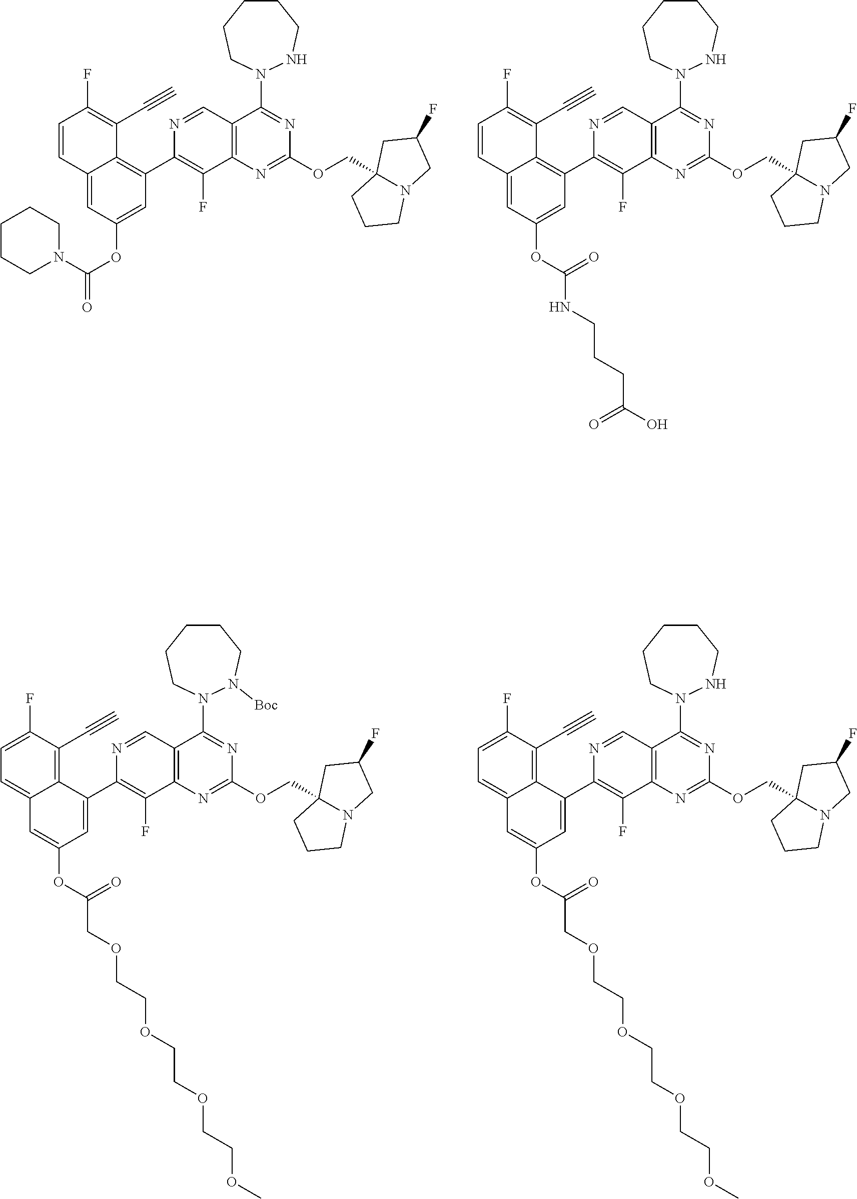 [SMILES: C#Cc1c(F)ccc2cc(OC(=O)N3CCCCC3)cc(-c3ncc4c(N5CCCCCN5)nc(OC[C@@]56CCCN5C[C@H](F)C6)nc4c3F)c12 ; C#Cc1c(F)ccc2cc(OC(=O)NCCCC(=O)O)cc(-c3ncc4c(N5CCCCCN5)nc(OC[C@@]56CCCN5C[C@H](F)C6)nc4c3F)c12 ; C#Cc1c(F)ccc2cc(OC(=O)COCCOCCOCCOC)cc(-c3ncc4c(N5CCCCCN5C(=O)OC(C)(C)C)nc(OC[C@@]56CCCN5C[C@H](F)C6)nc4c3F)c12 ; C#Cc1c(F)ccc2cc(OC(=O)COCCOCCOCCOC)cc(-c3ncc4c(N5CCCCCN5)nc(OC[C@@]56CCCN5C[C@H](F)C6)nc4c3F)c12]

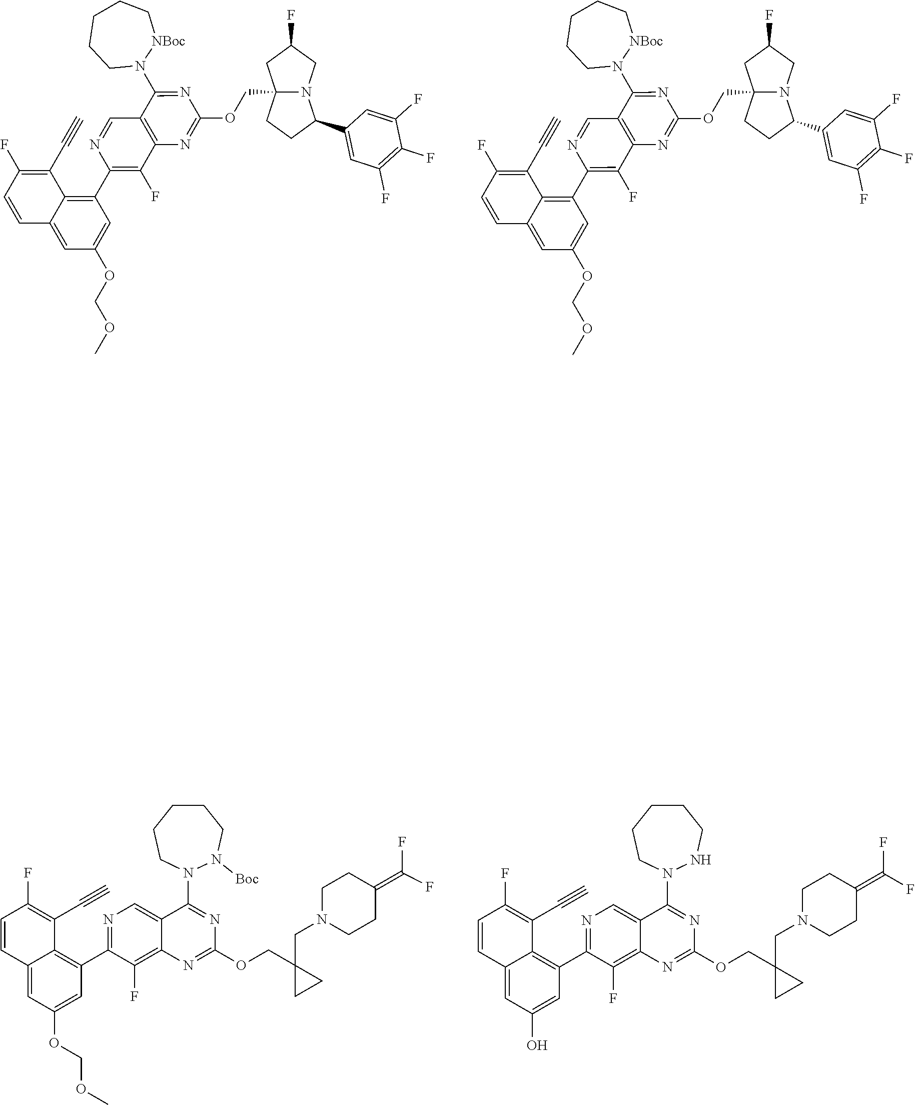 [SMILES: C#Cc1c(F)ccc2cc(OCOC)cc(-c3ncc4c(N5CCCCCN5C(=O)OC(C)(C)C)nc(OC[C@@]56CC[C@H](c7cc(F)c(F)c(F)c7)N5C[C@H](F)C6)nc4c3F)c12 ; C#Cc1c(F)ccc2cc(OCOC)cc(-c3ncc4c(N5CCCCCN5C(=O)OC(C)(C)C)nc(OC[C@@]56CC[C@@H](c7cc(F)c(F)c(F)c7)N5C[C@H](F)C6)nc4c3F)c12 ; C#Cc1c(F)ccc2cc(OCOC)cc(-c3ncc4c(N5CCCCCN5C(=O)OC(C)(C)C)nc(OCC5(CN6CCC(=C(F)F)CC6)CC5)nc4c3F)c12 ; C#Cc1c(F)ccc2cc(O)cc(-c3ncc4c(N5CCCCCN5)nc(OCC5(CN6CCC(=C(F)F)CC6)CC5)nc4c3F)c12]

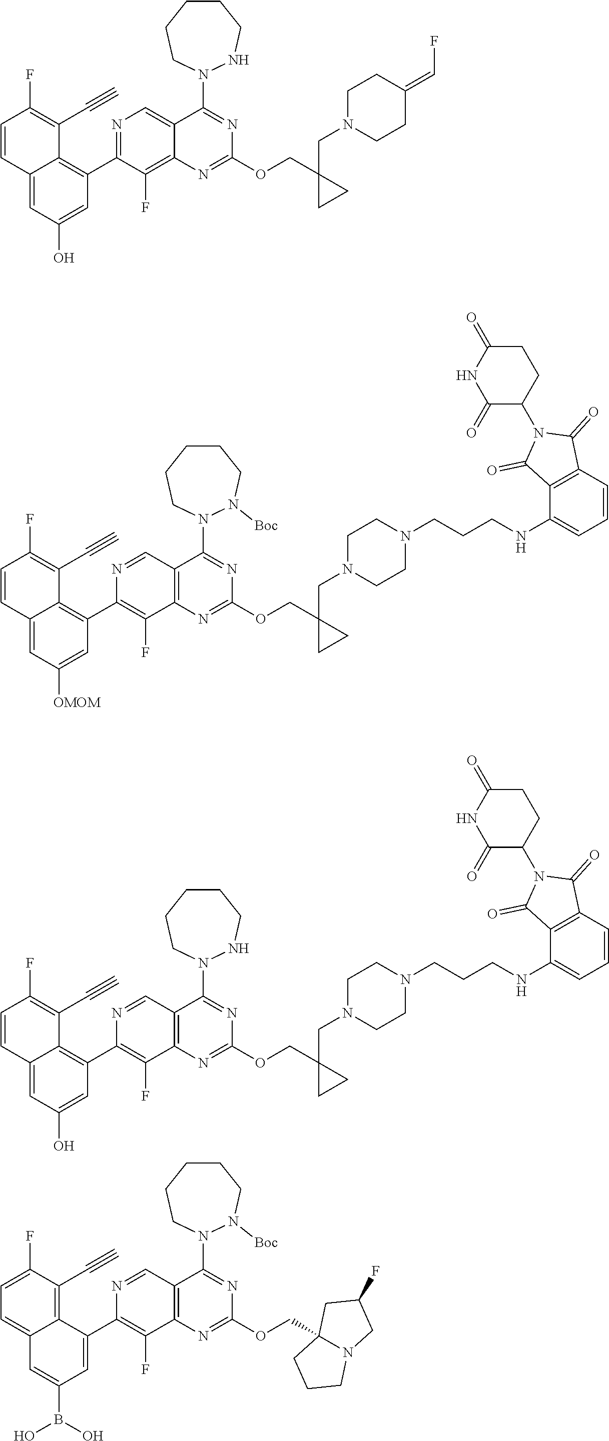

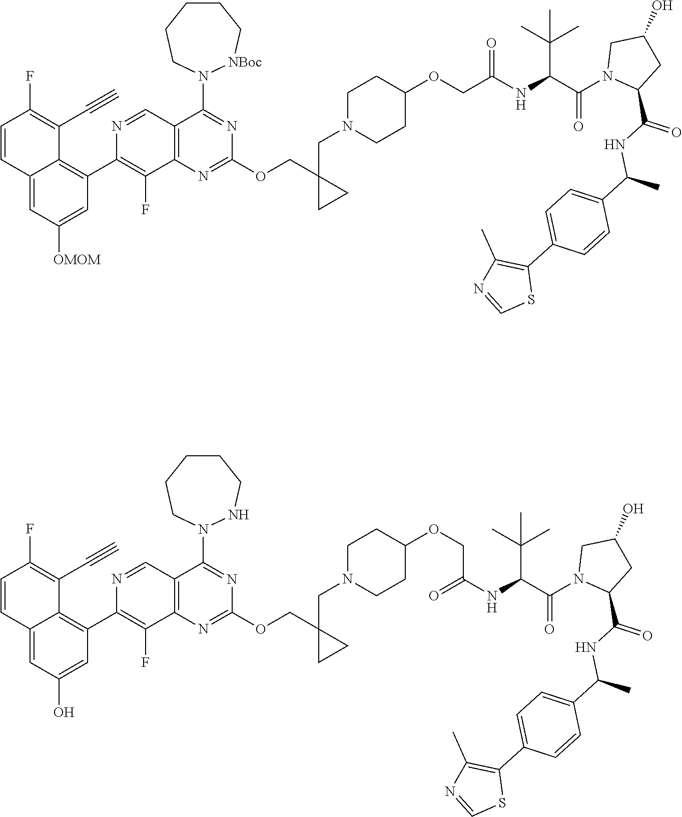

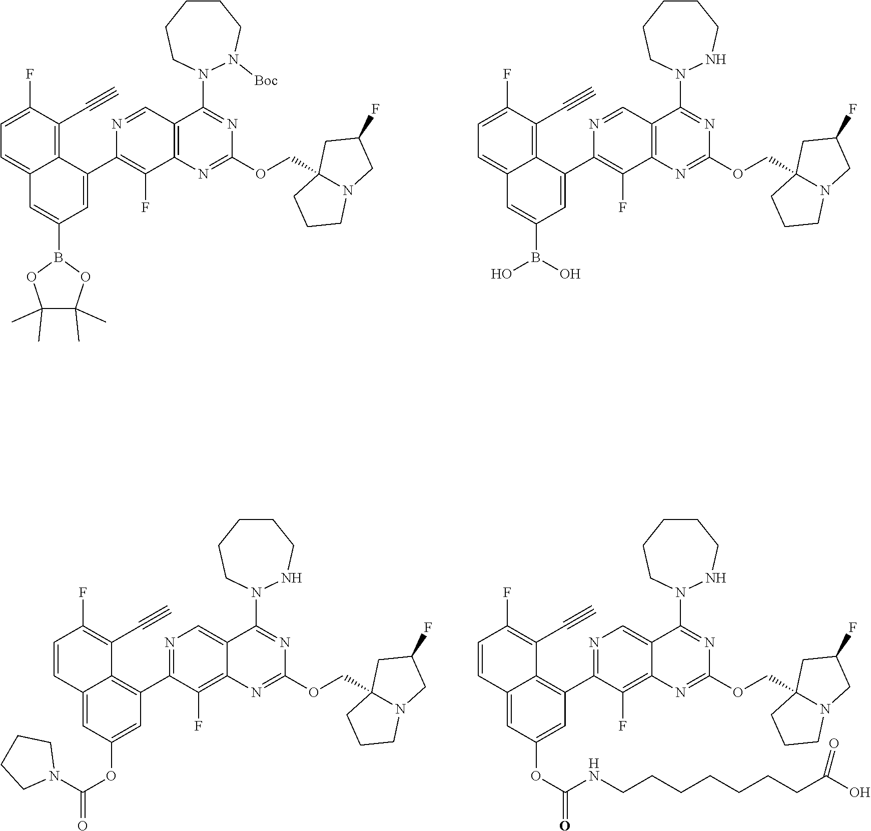 [SMILES: C#Cc1c(F)ccc2cc(B3OC(C)(C)C(C)(C)O3)cc(-c3ncc4c(N5CCCCCN5C(=O)OC(C)(C)C)nc(OC[C@@]56CCCN5C[C@H](F)C6)nc4c3F)c12 ; C#Cc1c(F)ccc2cc(B(O)O)cc(-c3ncc4c(N5CCCCCN5)nc(OC[C@@]56CCCN5C[C@H](F)C6)nc4c3F)c12 ; C#Cc1c(F)ccc2cc(OC(=O)N3CCCC3)cc(-c3ncc4c(N5CCCCCN5)nc(OC[C@@]56CCCN5C[C@H](F)C6)nc4c3F)c12 ; C#Cc1c(F)ccc2cc(OC(=O)NCCCCCCCC(=O)O)cc(-c3ncc4c(N5CCCCCN5)nc(OC[C@@]56CCCN5C[C@H](F)C6)nc4c3F)c12]

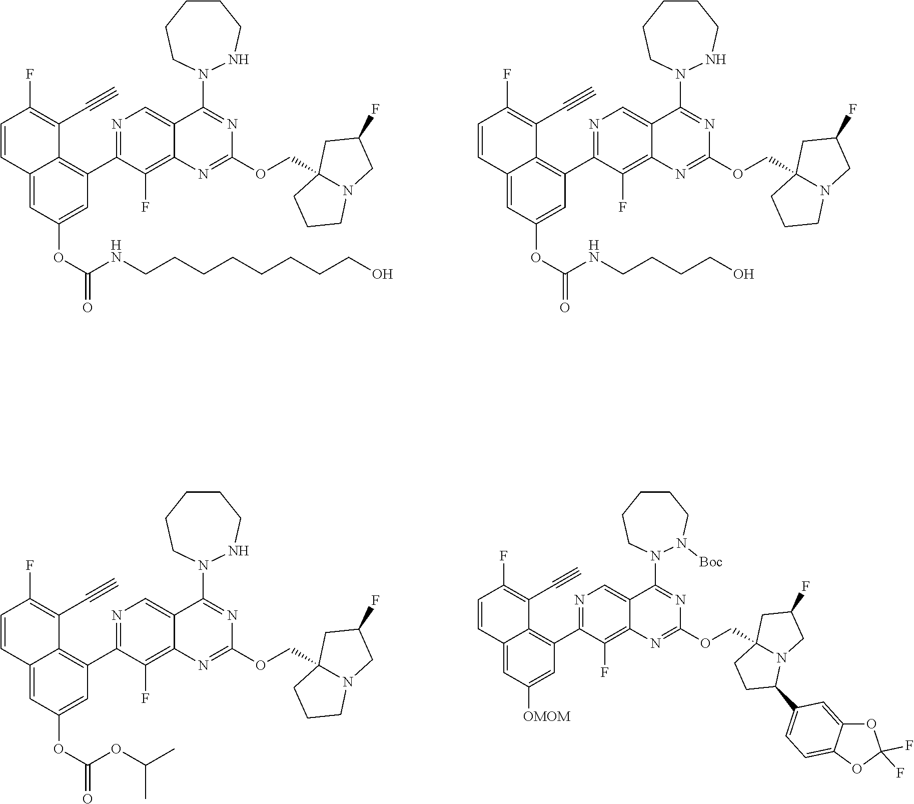

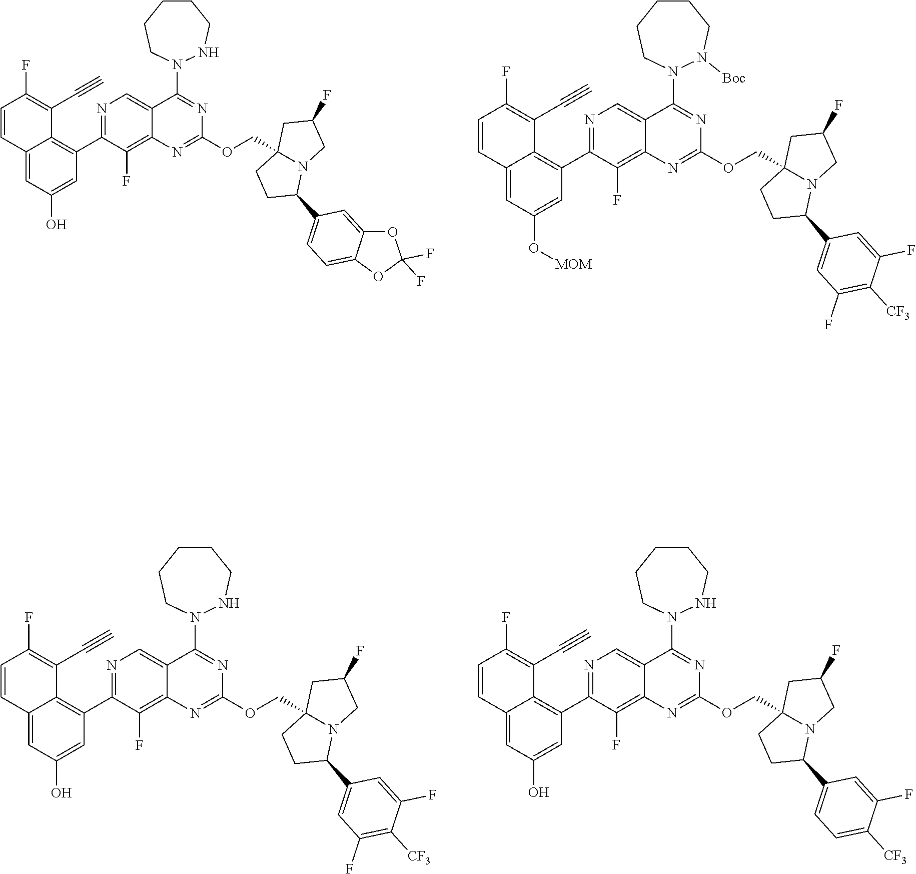

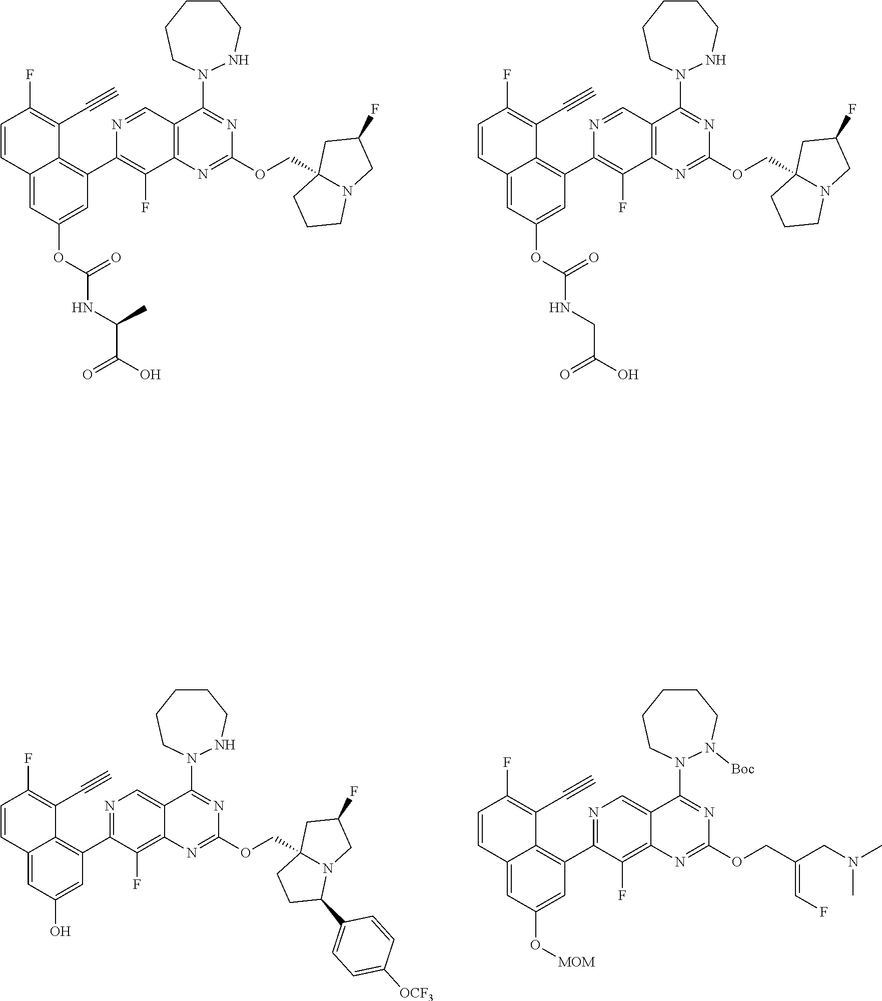

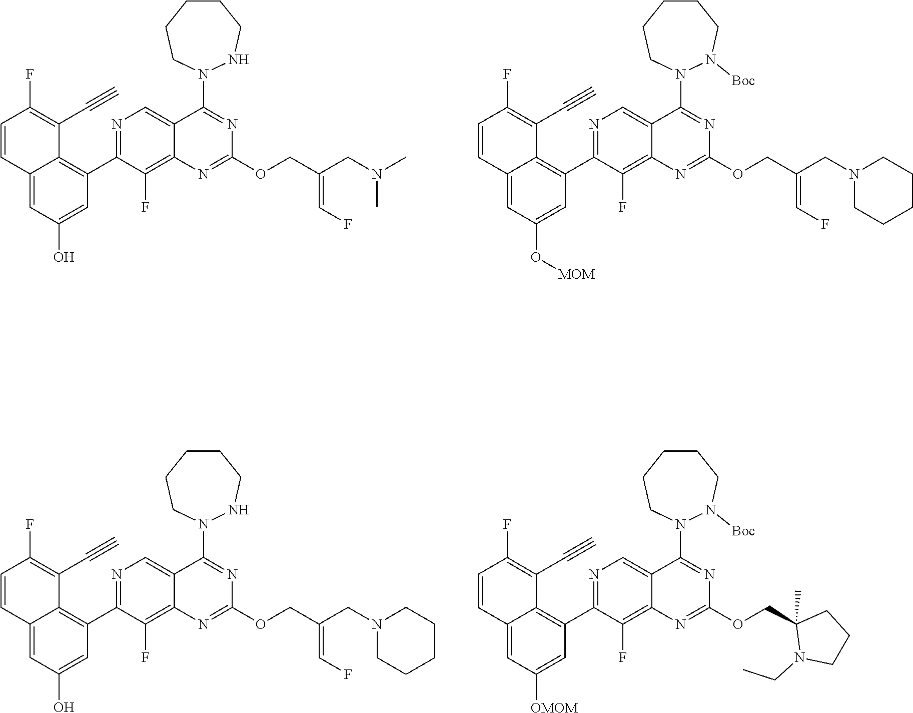

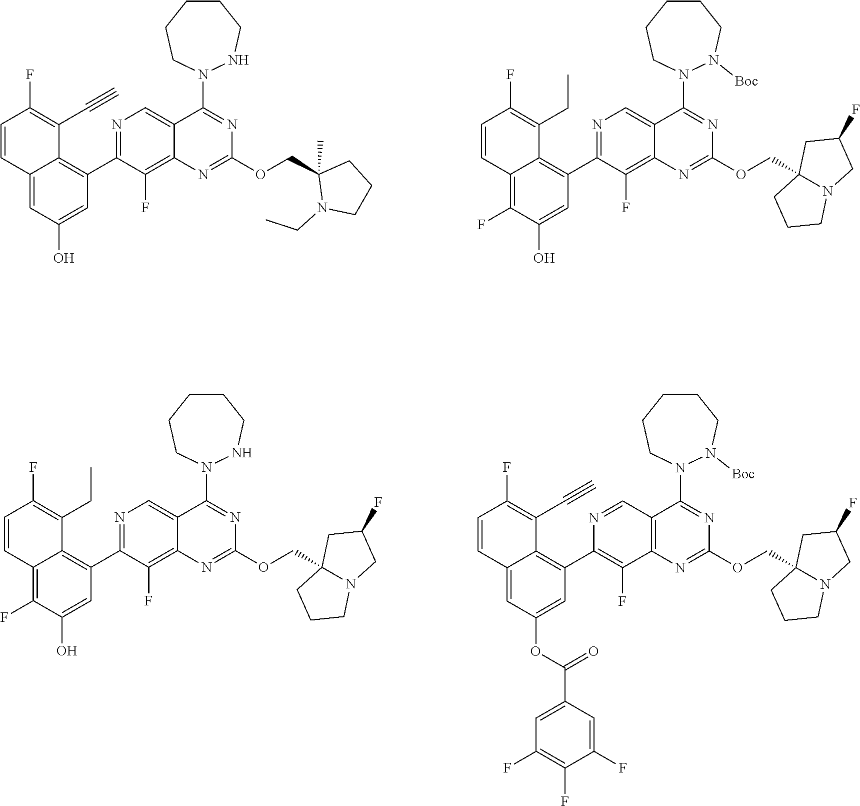 [SMILES: C#Cc1c(F)ccc2cc(O)cc(-c3ncc4c(N5CCCCCN5)nc(OC[C@@]5(C)CCCN5CC)nc4c3F)c12 ; CCc1c(F)ccc2c(F)c(O)cc(-c3ncc4c(N5CCCCCN5C(=O)OC(C)(C)C)nc(OC[C@@]56CCCN5C[C@H](F)C6)nc4c3F)c12 ; CCc1c(F)ccc2c(F)c(O)cc(-c3ncc4c(N5CCCCCN5)nc(OC[C@@]56CCCN5C[C@H](F)C6)nc4c3F)c12 ; C#Cc1c(F)ccc2cc(OC(=O)c3cc(F)c(F)c(F)c3)cc(-c3ncc4c(N5CCCCCN5C(=O)OC(C)(C)C)nc(OC[C@@]56CCCN5C[C@H](F)C6)nc4c3F)c12]

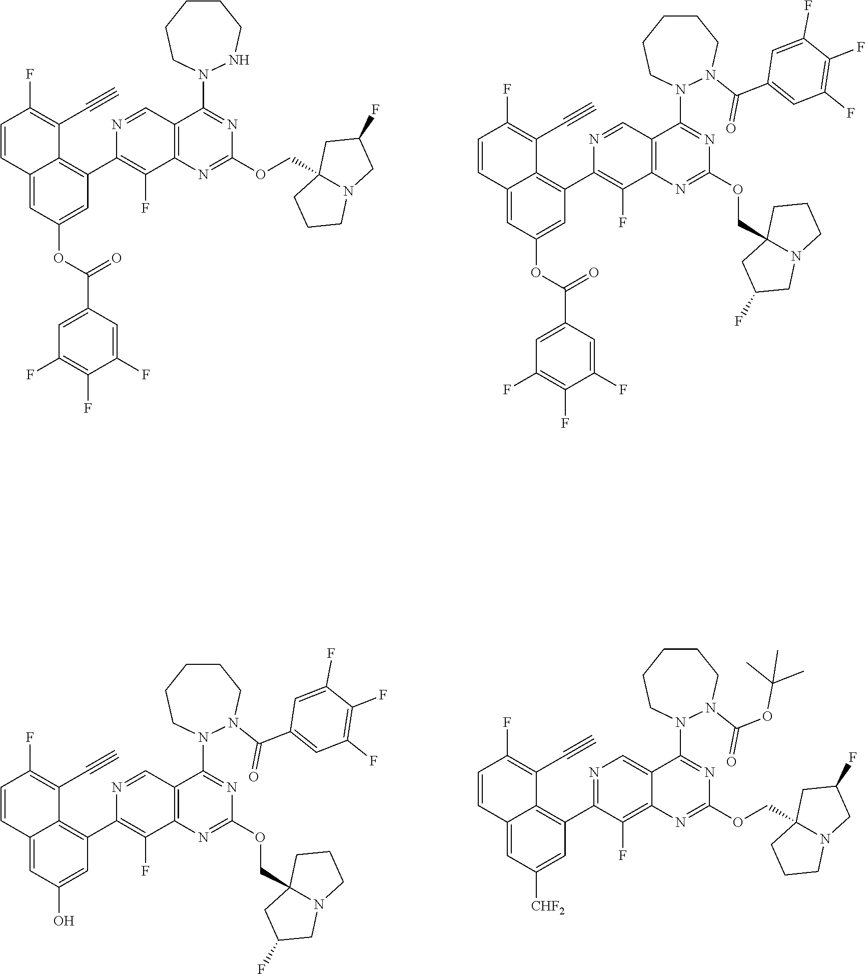 [SMILES: C#Cc1c(F)ccc2cc(OC(=O)c3cc(F)c(F)c(F)c3)cc(-c3ncc4c(N5CCCCCN5)nc(OC[C@@]56CCCN5C[C@H](F)C6)nc4c3F)c12 ; C#Cc1c(F)ccc2cc(OC(=O)c3cc(F)c(F)c(F)c3)cc(-c3ncc4c(N5CCCCCN5C(=O)c5cc(F)c(F)c(F)c5)nc(OC[C@@]56CCCN5C[C@H](F)C6)nc4c3F)c12 ; C#Cc1c(F)ccc2cc(O)cc(-c3ncc4c(N5CCCCCN5C(=O)c5cc(F)c(F)c(F)c5)nc(OC[C@@]56CCCN5C[C@H](F)C6)nc4c3F)c12 ; C#Cc1c(F)ccc2cc(C(F)F)cc(-c3ncc4c(N5CCCCCN5C(=O)OC(C)(C)C)nc(OC[C@@]56CCCN5C[C@H](F)C6)nc4c3F)c12]

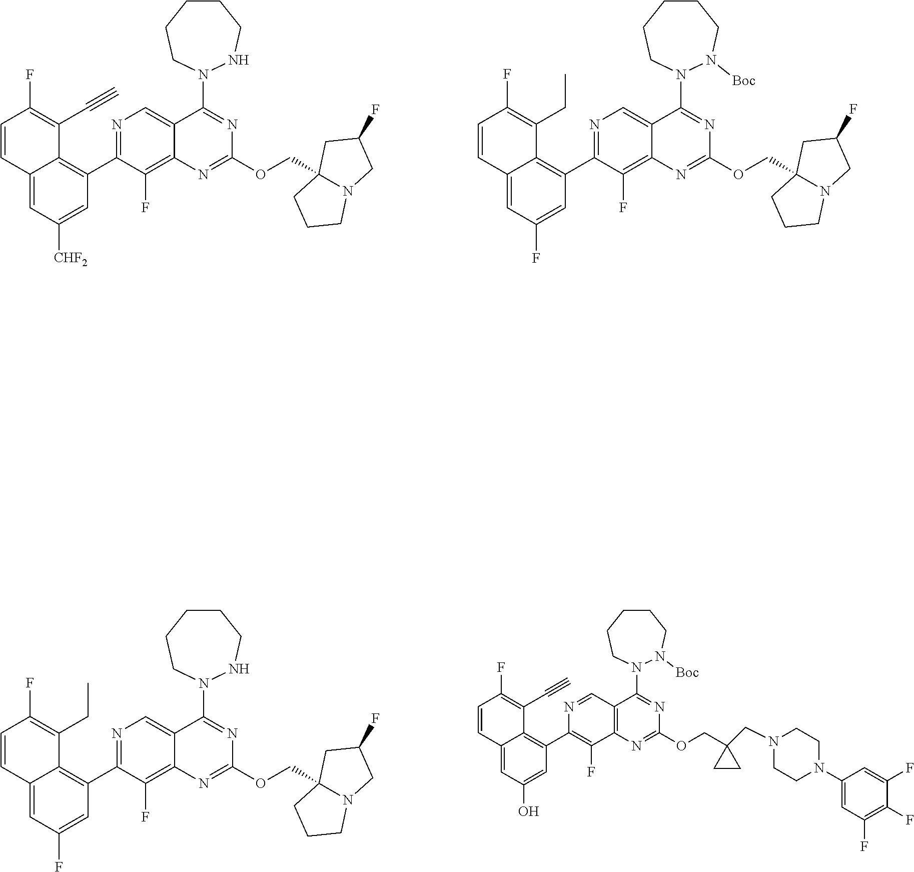 [SMILES: C#Cc1c(F)ccc2cc(C(F)F)cc(-c3ncc4c(N5CCCCCN5)nc(OC[C@@]56CCCN5C[C@H](F)C6)nc4c3F)c12 ; CCc1c(F)ccc2cc(F)cc(-c3ncc4c(N5CCCCCN5C(=O)OC(C)(C)C)nc(OC[C@@]56CCCN5C[C@H](F)C6)nc4c3F)c12 ; CCc1c(F)ccc2cc(F)cc(-c3ncc4c(N5CCCCCN5)nc(OC[C@@]56CCCN5C[C@H](F)C6)nc4c3F)c12 ; C#Cc1c(F)ccc2cc(O)cc(-c3ncc4c(N5CCCCCN5C(=O)OC(C)(C)C)nc(OCC5(CN6CCN(c7cc(F)c(F)c(F)c7)CC6)CC5)nc4c3F)c12]

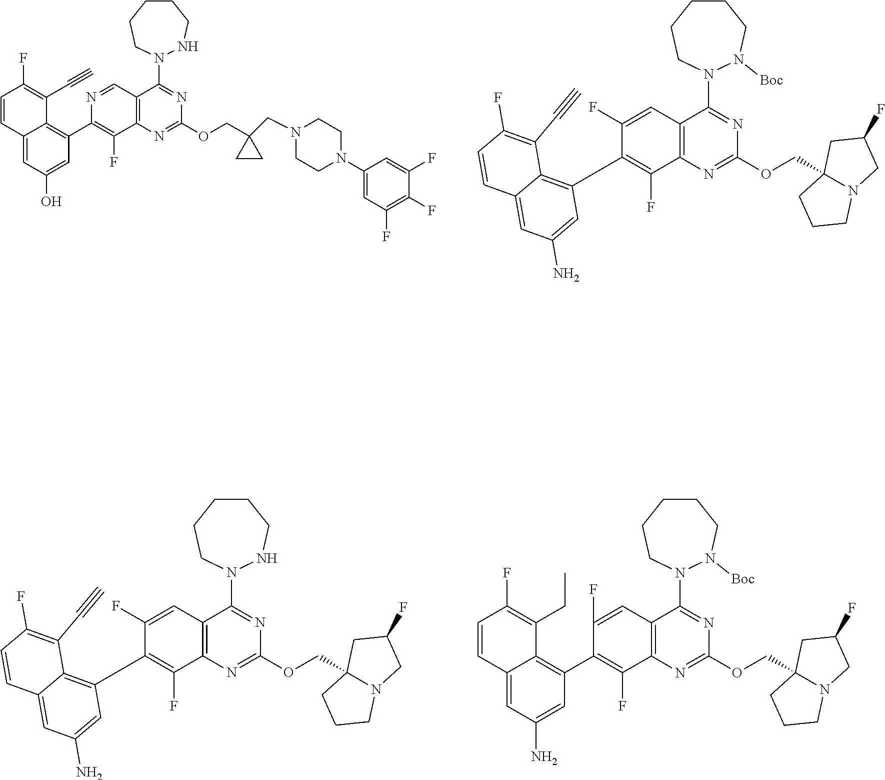 [SMILES: C#Cc1c(F)ccc2cc(O)cc(-c3ncc4c(N5CCCCCN5)nc(OCC5(CN6CCN(c7cc(F)c(F)c(F)c7)CC6)CC5)nc4c3F)c12 ; C#Cc1c(F)ccc2cc(N)cc(-c3c(F)cc4c(N5CCCCCN5C(=O)OC(C)(C)C)nc(OC[C@@]56CCCN5C[C@H](F)C6)nc4c3F)c12 ; C#Cc1c(F)ccc2cc(N)cc(-c3c(F)cc4c(N5CCCCCN5)nc(OC[C@@]56CCCN5C[C@H](F)C6)nc4c3F)c12 ; CCc1c(F)ccc2cc(N)cc(-c3c(F)cc4c(N5CCCCCN5C(=O)OC(C)(C)C)nc(OC[C@@]56CCCN5C[C@H](F)C6)nc4c3F)c12]

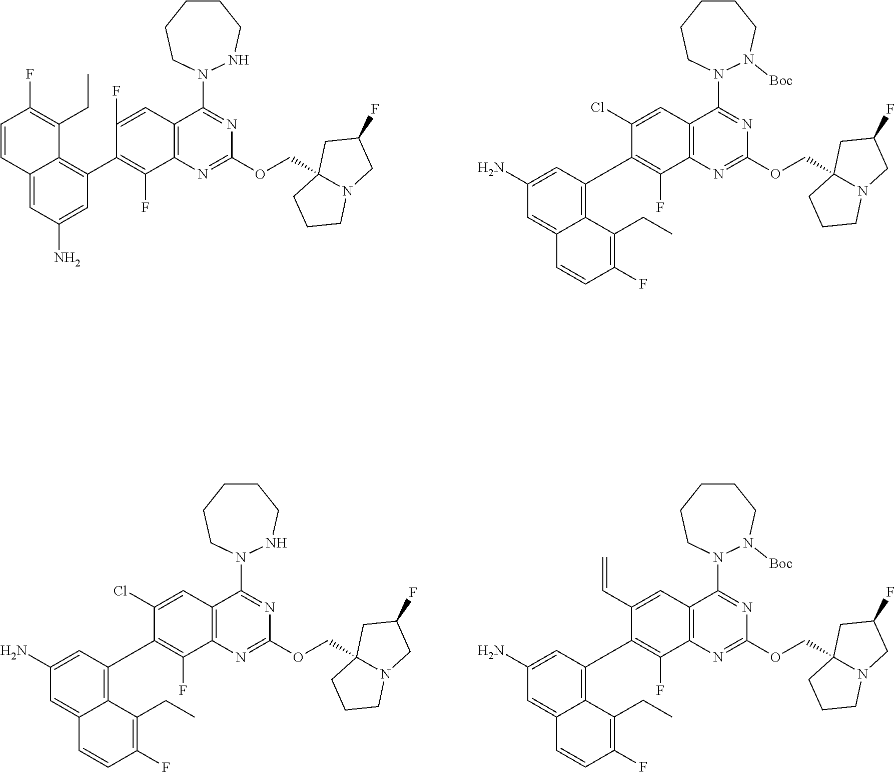 [SMILES: CCc1c(F)ccc2cc(N)cc(-c3c(F)cc4c(N5CCCCCN5)nc(OC[C@@]56CCCN5C[C@H](F)C6)nc4c3F)c12 ; CCc1c(F)ccc2cc(N)cc(-c3c(Cl)cc4c(N5CCCCCN5C(=O)OC(C)(C)C)nc(OC[C@@]56CCCN5C[C@H](F)C6)nc4c3F)c12 ; CCc1c(F)ccc2cc(N)cc(-c3c(Cl)cc4c(N5CCCCCN5)nc(OC[C@@]56CCCN5C[C@H](F)C6)nc4c3F)c12 ; C=Cc1cc2c(N3CCCCCN3C(=O)OC(C)(C)C)nc(OC[C@@]34CCCN3C[C@H](F)C4)nc2c(F)c1-c1cc(N)cc2ccc(F)c(CC)c12]

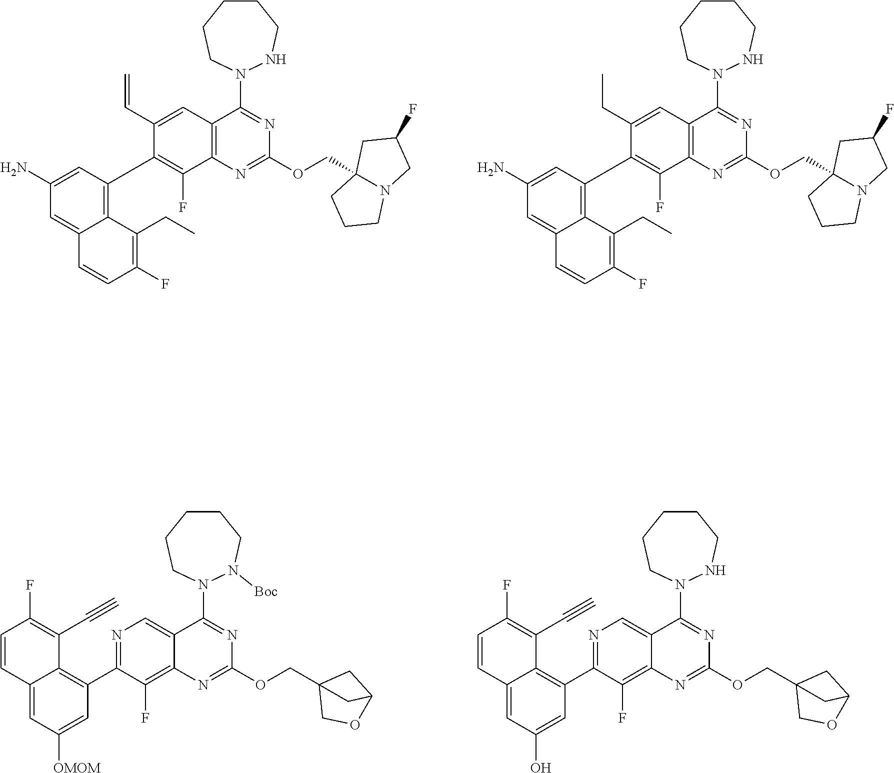

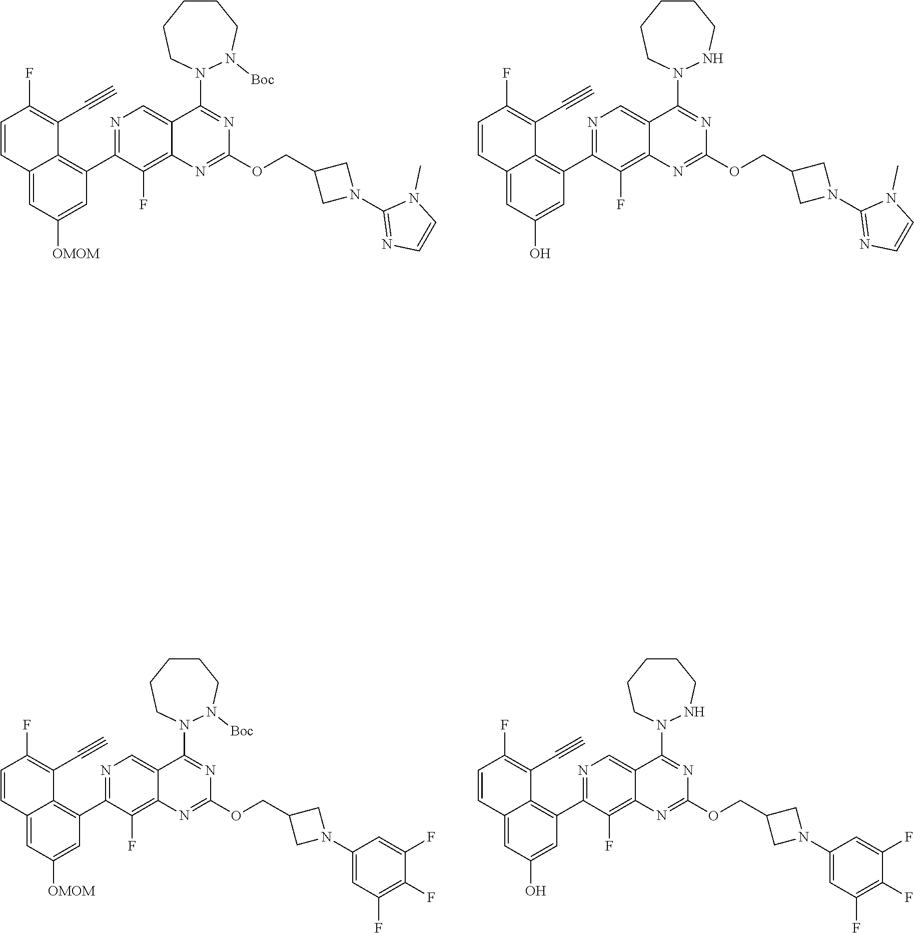

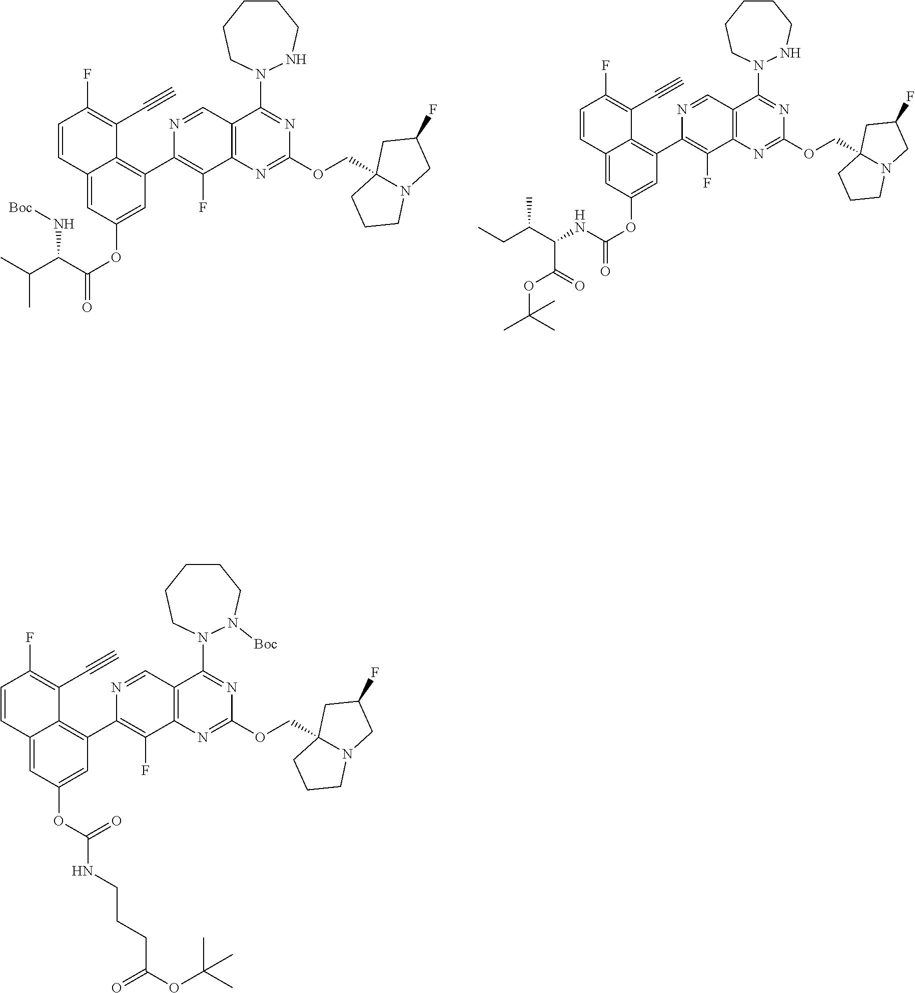 [SMILES: C#Cc1c(F)ccc2cc(OC(=O)[C@@H](NC(=O)OC(C)(C)C)C(C)C)cc(-c3ncc4c(N5CCCCCN5)nc(OC[C@@]56CCCN5C[C@H](F)C6)nc4c3F)c12 ; C#Cc1c(F)ccc2cc(OC(=O)N[C@H](C(=O)OC(C)(C)C)[C@@H](C)CC)cc(-c3ncc4c(N5CCCCCN5)nc(OC[C@@]56CCCN5C[C@H](F)C6)nc4c3F)c12 ; C#Cc1c(F)ccc2cc(OC(=O)NCCCC(=O)OC(C)(C)C)cc(-c3ncc4c(N5CCCCCN5C(=O)OC(C)(C)C)nc(OC[C@@]56CCCN5C[C@H](F)C6)nc4c3F)c12]

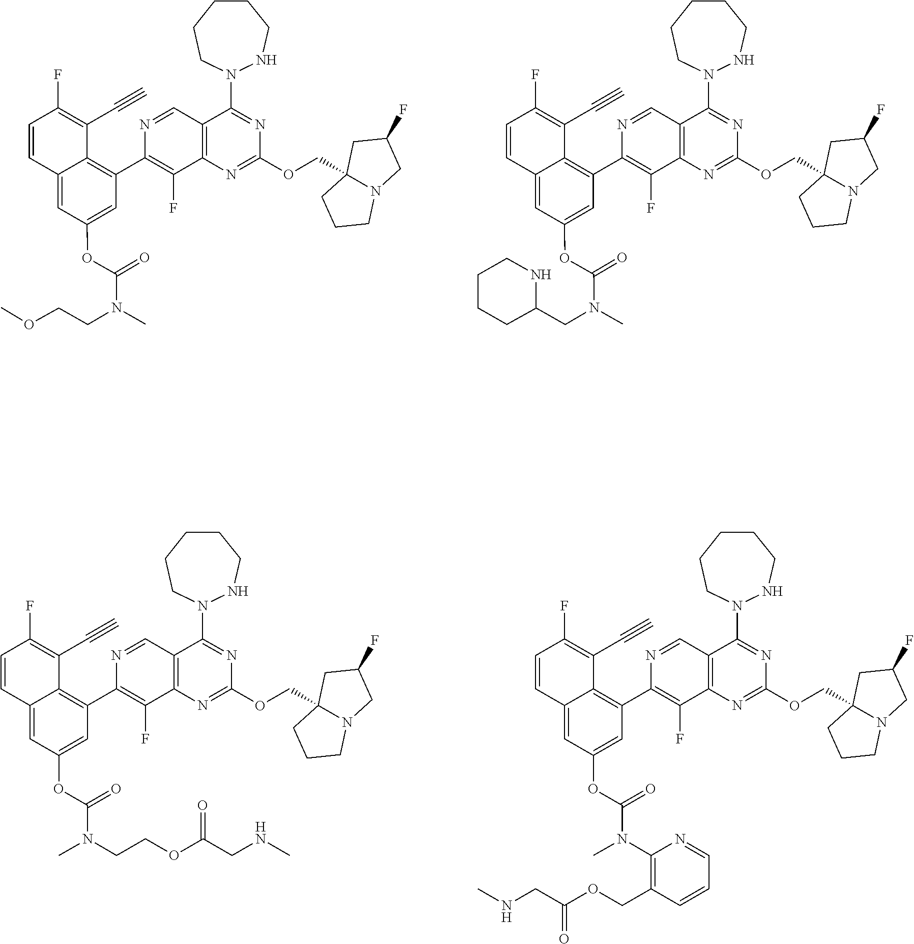 [SMILES: C#Cc1c(F)ccc2cc(OC(=O)N(C)CCOC)cc(-c3ncc4c(N5CCCCCN5)nc(OC[C@@]56CCCN5C[C@H](F)C6)nc4c3F)c12 ; C#Cc1c(F)ccc2cc(OC(=O)N(C)CC3CCCCN3)cc(-c3ncc4c(N5CCCCCN5)nc(OC[C@@]56CCCN5C[C@H](F)C6)nc4c3F)c12 ; C#Cc1c(F)ccc2cc(OC(=O)N(C)CCOC(=O)CNC)cc(-c3ncc4c(N5CCCCCN5)nc(OC[C@@]56CCCN5C[C@H](F)C6)nc4c3F)c12 ; C#Cc1c(F)ccc2cc(OC(=O)N(C)c3ncccc3COC(=O)CNC)cc(-c3ncc4c(N5CCCCCN5)nc(OC[C@@]56CCCN5C[C@H](F)C6)nc4c3F)c12]

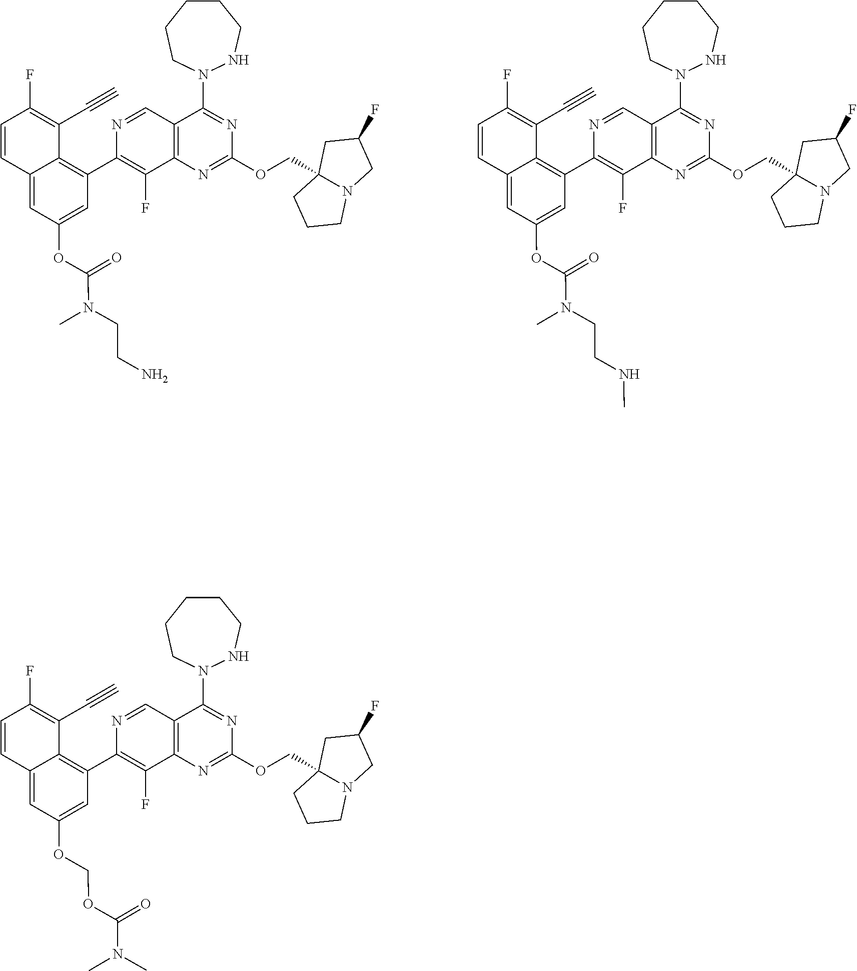 [SMILES: C#Cc1c(F)ccc2cc(OC(=O)N(C)CCN)cc(-c3ncc4c(N5CCCCCN5)nc(OC[C@@]56CCCN5C[C@H](F)C6)nc4c3F)c12 ; C#Cc1c(F)ccc2cc(OC(=O)N(C)CCNC)cc(-c3ncc4c(N5CCCCCN5)nc(OC[C@@]56CCCN5C[C@H](F)C6)nc4c3F)c12 ; C#Cc1c(F)ccc2cc(OCOC(=O)N(C)C)cc(-c3ncc4c(N5CCCCCN5)nc(OC[C@@]56CCCN5C[C@H](F)C6)nc4c3F)c12]

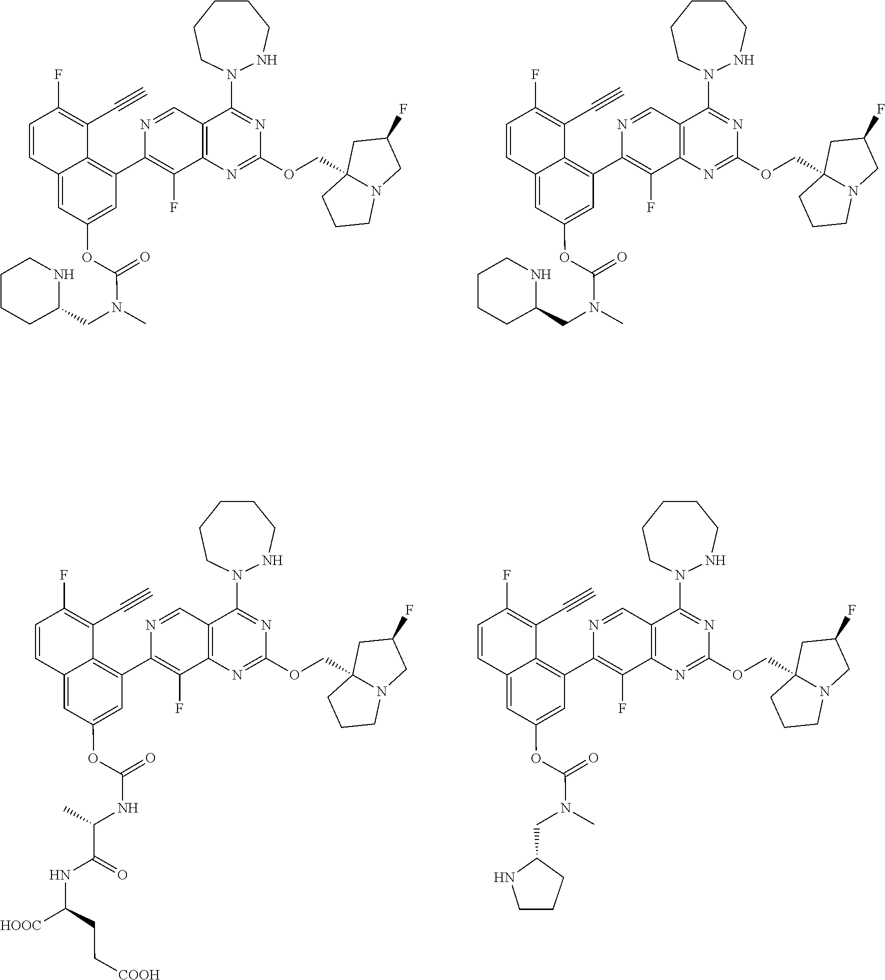 [SMILES: C#Cc1c(F)ccc2cc(OC(=O)N(C)C[C@@H]3CCCCN3)cc(-c3ncc4c(N5CCCCCN5)nc(OC[C@@]56CCCN5C[C@H](F)C6)nc4c3F)c12 ; C#Cc1c(F)ccc2cc(OC(=O)N(C)C[C@H]3CCCCN3)cc(-c3ncc4c(N5CCCCCN5)nc(OC[C@@]56CCCN5C[C@H](F)C6)nc4c3F)c12 ; C#Cc1c(F)ccc2cc(OC(=O)N[C@@H](C)C(=O)N[C@@H](CCC(=O)O)C(=O)O)cc(-c3ncc4c(N5CCCCCN5)nc(OC[C@@]56CCCN5C[C@H](F)C6)nc4c3F)c12 ; C#Cc1c(F)ccc2cc(OC(=O)N(C)C[C@@H]3CCCN3)cc(-c3ncc4c(N5CCCCCN5)nc(OC[C@@]56CCCN5C[C@H](F)C6)nc4c3F)c12]

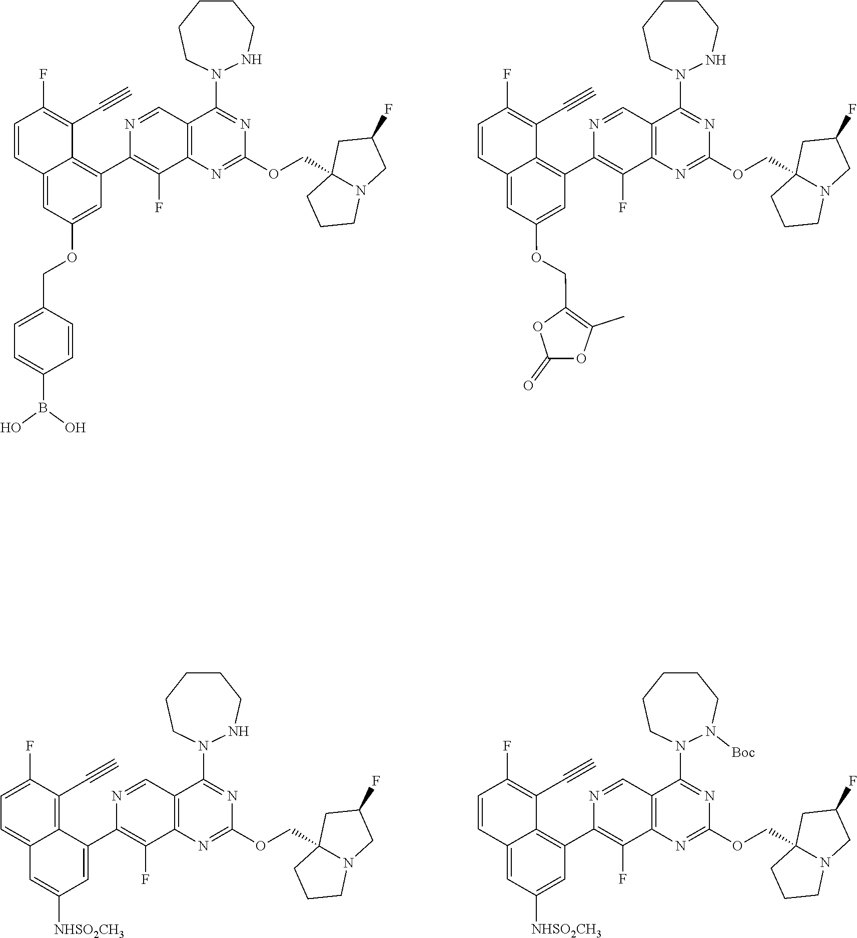

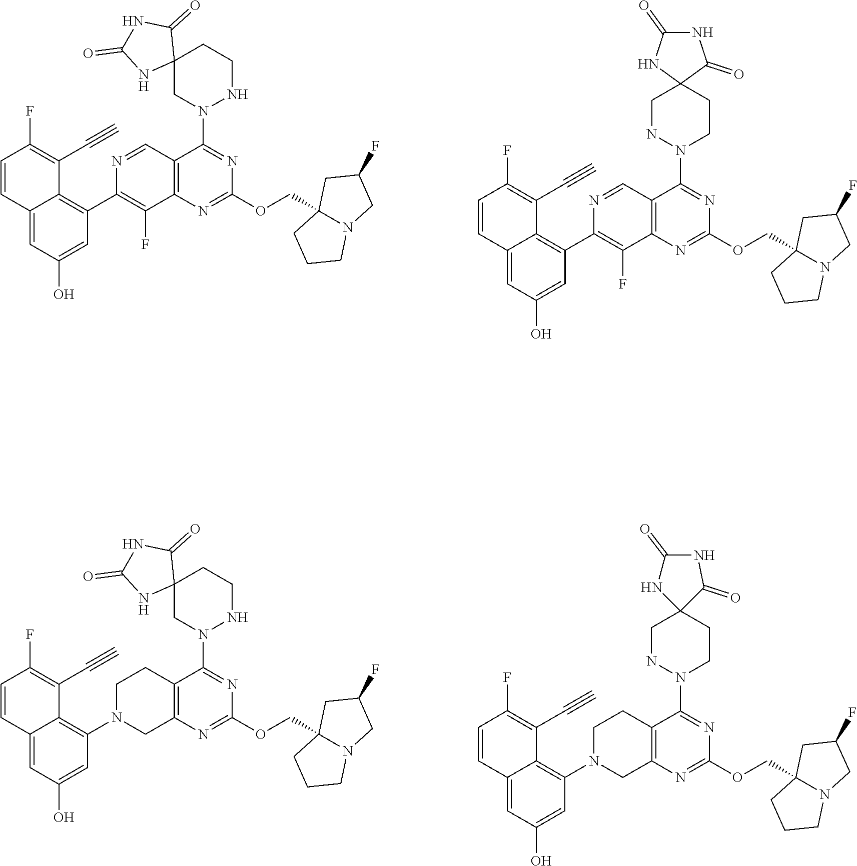 [SMILES: C#Cc1c(F)ccc2cc(O)cc(-c3ncc4c(N5CC6(CCN5)NC(=O)NC6=O)nc(OC[C@@]56CCCN5C[C@H](F)C6)nc4c3F)c12 ; C#Cc1c(F)ccc2cc(O)cc(-c3ncc4c(N5CCC6(CN5)NC(=O)NC6=O)nc(OC[C@@]56CCCN5C[C@H](F)C6)nc4c3F)c12 ; C#Cc1c(F)ccc2cc(O)cc(N3CCc4c(nc(OC[C@@]56CCCN5C[C@H](F)C6)nc4N4CC5(CCN4)NC(=O)NC5=O)C3)c12 ; C#Cc1c(F)ccc2cc(O)cc(N3CCc4c(nc(OC[C@@]56CCCN5C[C@H](F)C6)nc4N4CCC5(CN4)NC(=O)NC5=O)C3)c12]

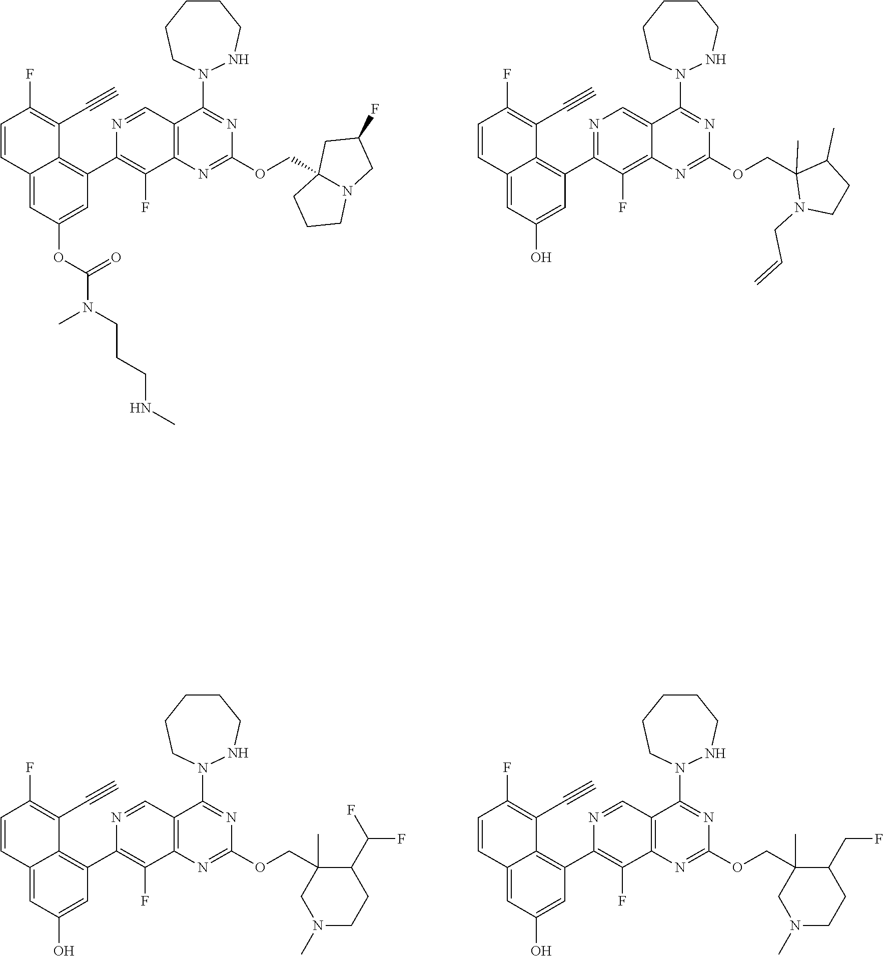 [SMILES: C#Cc1c(F)ccc2cc(OC(=O)N(C)CCCNC)cc(-c3ncc4c(N5CCCCCN5)nc(OC[C@@]56CCCN5C[C@H](F)C6)nc4c3F)c12 ; C#Cc1c(F)ccc2cc(O)cc(-c3ncc4c(N5CCCCCN5)nc(OCC5(C)C(C)CCN5CC=C)nc4c3F)c12 ; C#Cc1c(F)ccc2cc(O)cc(-c3ncc4c(N5CCCCCN5)nc(OCC5(C)CN(C)CCC5C(F)F)nc4c3F)c12 ; C#Cc1c(F)ccc2cc(O)cc(-c3ncc4c(N5CCCCCN5)nc(OCC5(C)CN(C)CCC5CF)nc4c3F)c12]

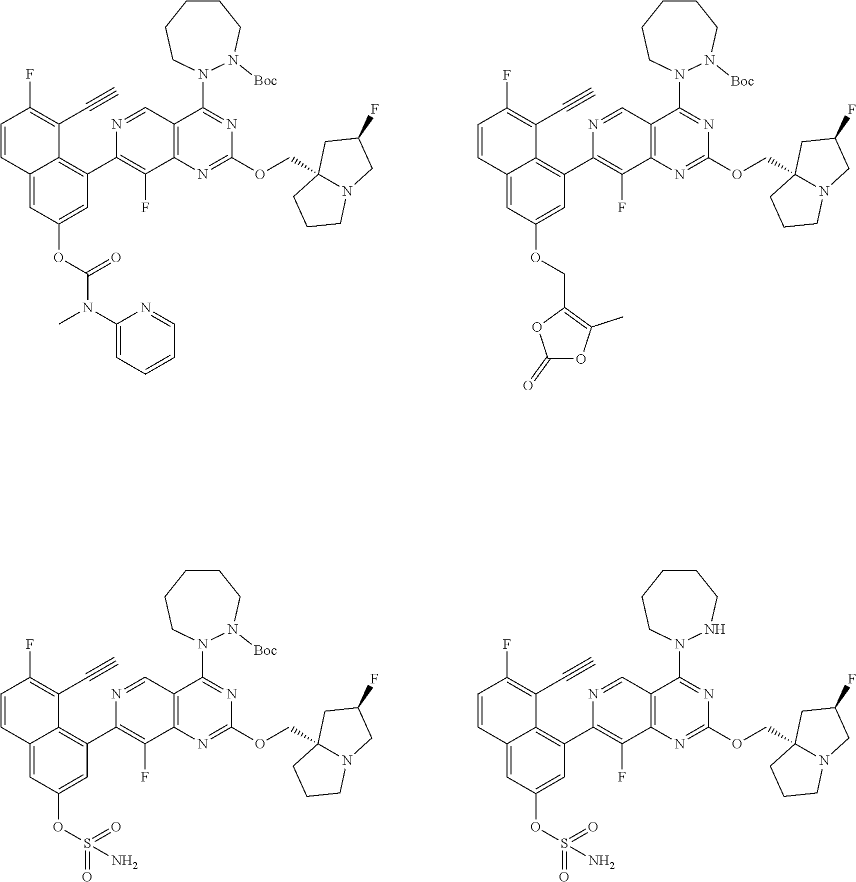 [SMILES: C#Cc1c(F)ccc2cc(OC(=O)N(C)c3ccccn3)cc(-c3ncc4c(N5CCCCCN5C(=O)OC(C)(C)C)nc(OC[C@@]56CCCN5C[C@H](F)C6)nc4c3F)c12 ; C#Cc1c(F)ccc2cc(OCc3oc(=O)oc3C)cc(-c3ncc4c(N5CCCCCN5C(=O)OC(C)(C)C)nc(OC[C@@]56CCCN5C[C@H](F)C6)nc4c3F)c12 ; C#Cc1c(F)ccc2cc(OS(N)(=O)=O)cc(-c3ncc4c(N5CCCCCN5C(=O)OC(C)(C)C)nc(OC[C@@]56CCCN5C[C@H](F)C6)nc4c3F)c12 ; C#Cc1c(F)ccc2cc(OS(N)(=O)=O)cc(-c3ncc4c(N5CCCCCN5)nc(OC[C@@]56CCCN5C[C@H](F)C6)nc4c3F)c12]

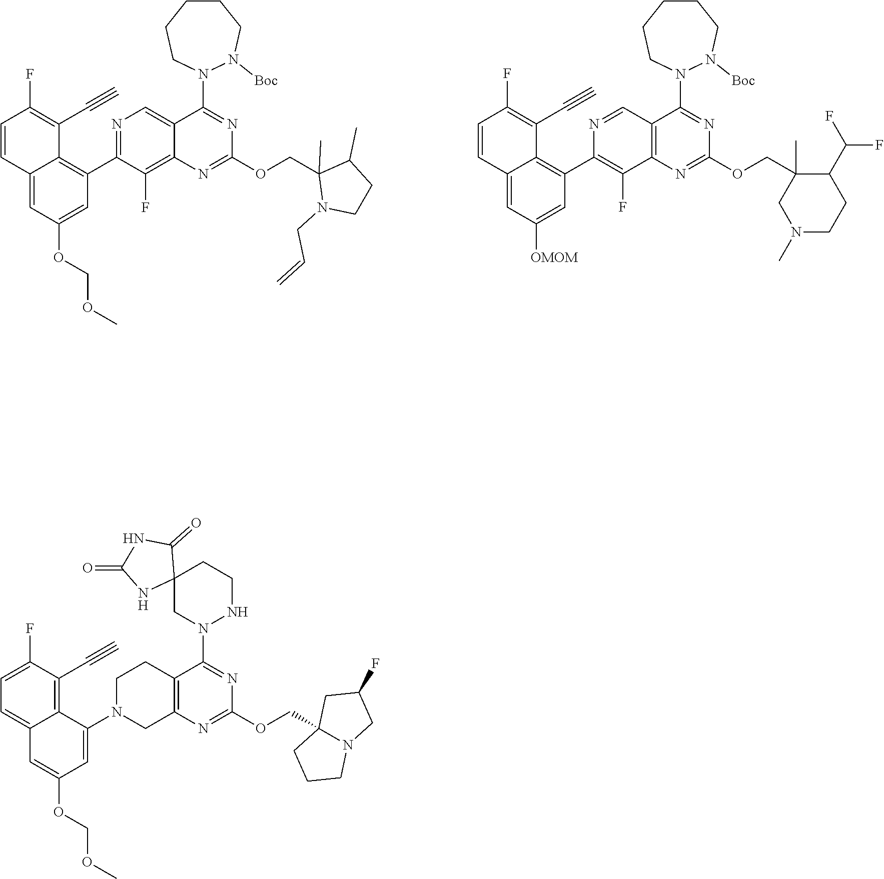

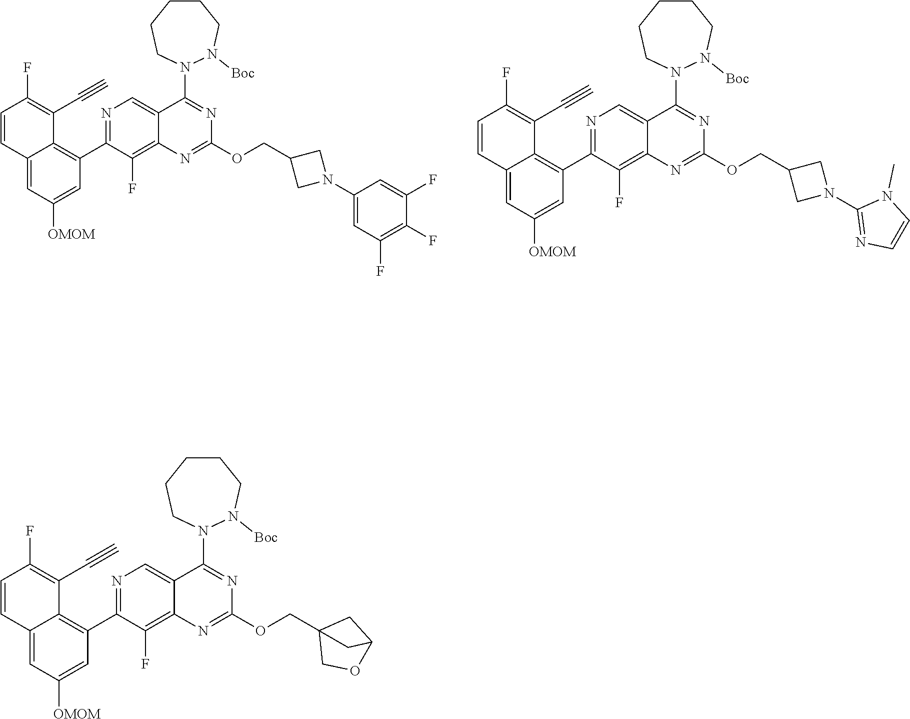

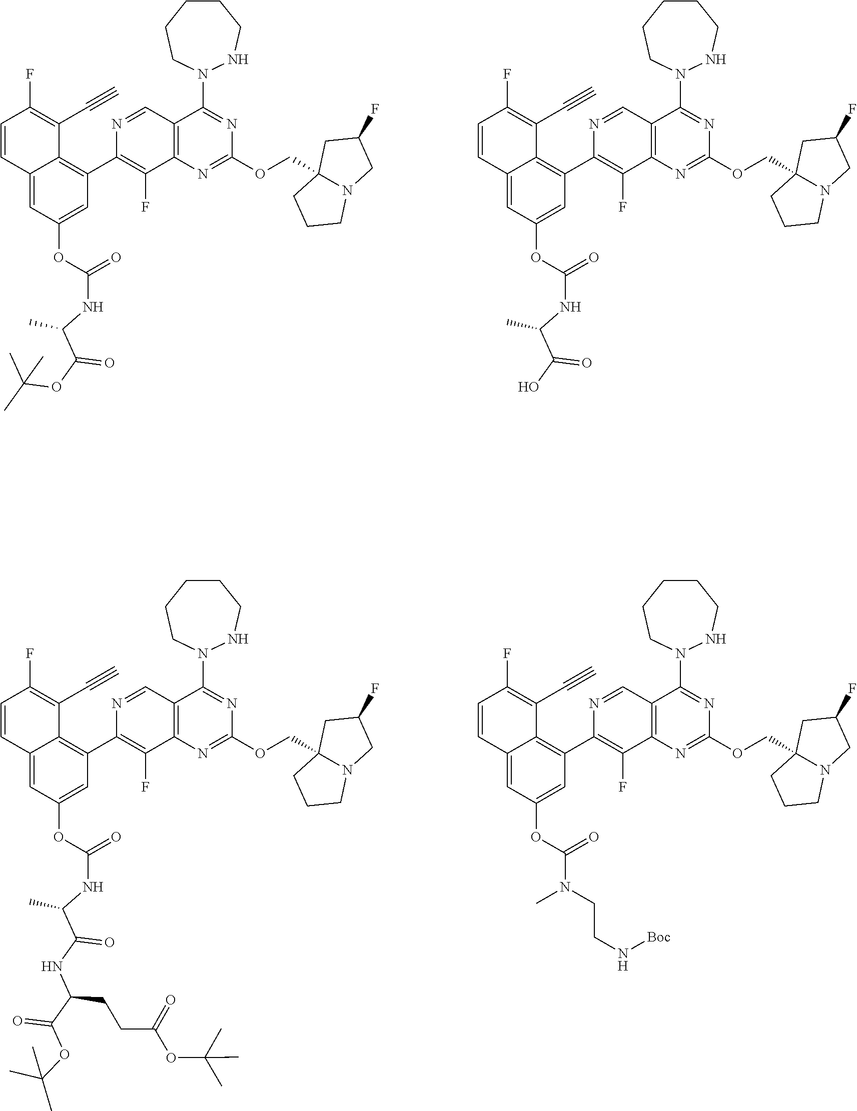 [SMILES: C#Cc1c(F)ccc2cc(OC(=O)N[C@@H](C)C(=O)OC(C)(C)C)cc(-c3ncc4c(N5CCCCCN5)nc(OC[C@@]56CCCN5C[C@H](F)C6)nc4c3F)c12 ; C#Cc1c(F)ccc2cc(OC(=O)N[C@@H](C)C(=O)O)cc(-c3ncc4c(N5CCCCCN5)nc(OC[C@@]56CCCN5C[C@H](F)C6)nc4c3F)c12 ; C#Cc1c(F)ccc2cc(OC(=O)N[C@@H](C)C(=O)N[C@@H](CCC(=O)OC(C)(C)C)C(=O)OC(C)(C)C)cc(-c3ncc4c(N5CCCCCN5)nc(OC[C@@]56CCCN5C[C@H](F)C6)nc4c3F)c12 ; C#Cc1c(F)ccc2cc(OC(=O)N(C)CCNC(=O)OC(C)(C)C)cc(-c3ncc4c(N5CCCCCN5)nc(OC[C@@]56CCCN5C[C@H](F)C6)nc4c3F)c12]

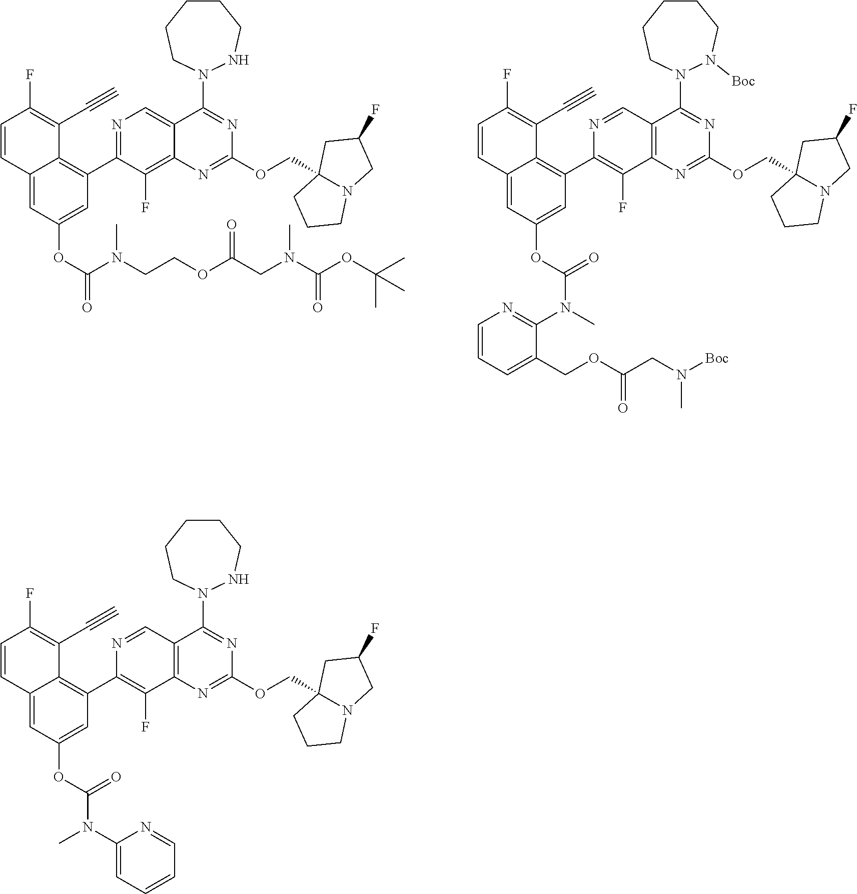 [SMILES: C#Cc1c(F)ccc2cc(OC(=O)N(C)CCOC(=O)CN(C)C(=O)OC(C)(C)C)cc(-c3ncc4c(N5CCCCCN5)nc(OC[C@@]56CCCN5C[C@H](F)C6)nc4c3F)c12 ; C#Cc1c(F)ccc2cc(OC(=O)N(C)c3ncccc3COC(=O)CN(C)C(=O)OC(C)(C)C)cc(-c3ncc4c(N5CCCCCN5C(=O)OC(C)(C)C)nc(OC[C@@]56CCCN5C[C@H](F)C6)nc4c3F)c12 ; C#Cc1c(F)ccc2cc(OC(=O)N(C)c3ccccn3)cc(-c3ncc4c(N5CCCCCN5)nc(OC[C@@]56CCCN5C[C@H](F)C6)nc4c3F)c12]

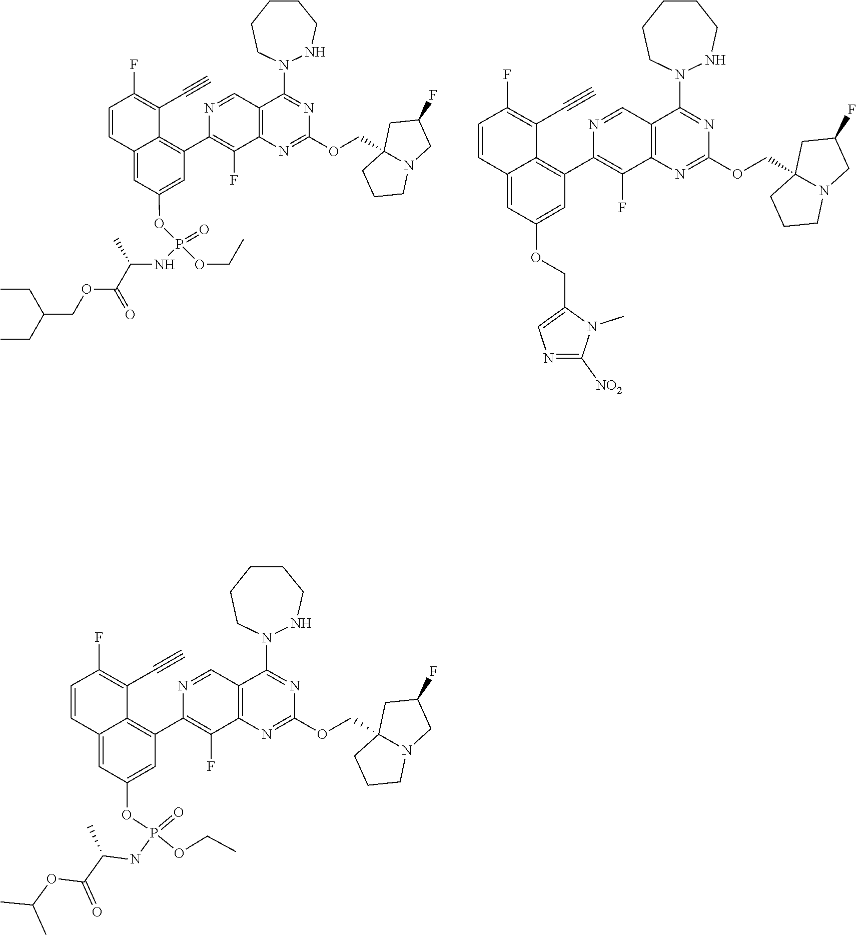 [SMILES: C#Cc1c(F)ccc2cc(OP(=O)(N[C@@H](C)C(=O)OCC(CC)CC)OCC)cc(-c3ncc4c(N5CCCCCN5)nc(OC[C@@]56CCCN5C[C@H](F)C6)nc4c3F)c12 ; C#Cc1c(F)ccc2cc(OCc3cnc([N+](=O)[O-])n3C)cc(-c3ncc4c(N5CCCCCN5)nc(OC[C@@]56CCCN5C[C@H](F)C6)nc4c3F)c12 ; C#Cc1c(F)ccc2cc(OP(=O)(N[C@@H](C)C(=O)OC(C)C)OCC)cc(-c3ncc4c(N5CCCCCN5)nc(OC[C@@]56CCCN5C[C@H](F)C6)nc4c3F)c12]

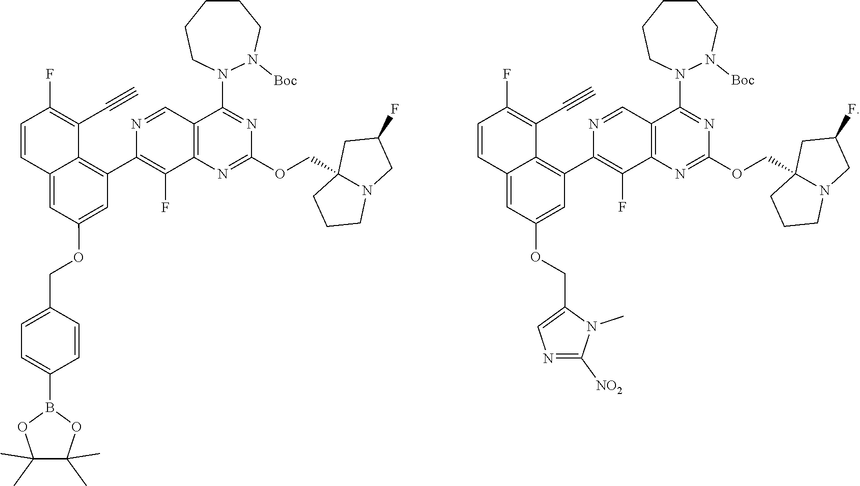 [SMILES: C#Cc1c(F)ccc2cc(OCc3ccc(B4OC(C)(C)C(C)(C)O4)cc3)cc(-c3ncc4c(N5CCCCCN5C(=O)OC(C)(C)C)nc(OC[C@@]56CCCN5C[C@H](F)C6)nc4c3F)c12 ; C#Cc1c(F)ccc2cc(OCc3cnc([N+](=O)[O-])n3C)cc(-c3ncc4c(N5CCCCCN5C(=O)OC(C)(C)C)nc(OC[C@@]56CCCN5C[C@H](F)C6)nc4c3F)c12]

The present disclosure also provides a compound having any one of the following structures:

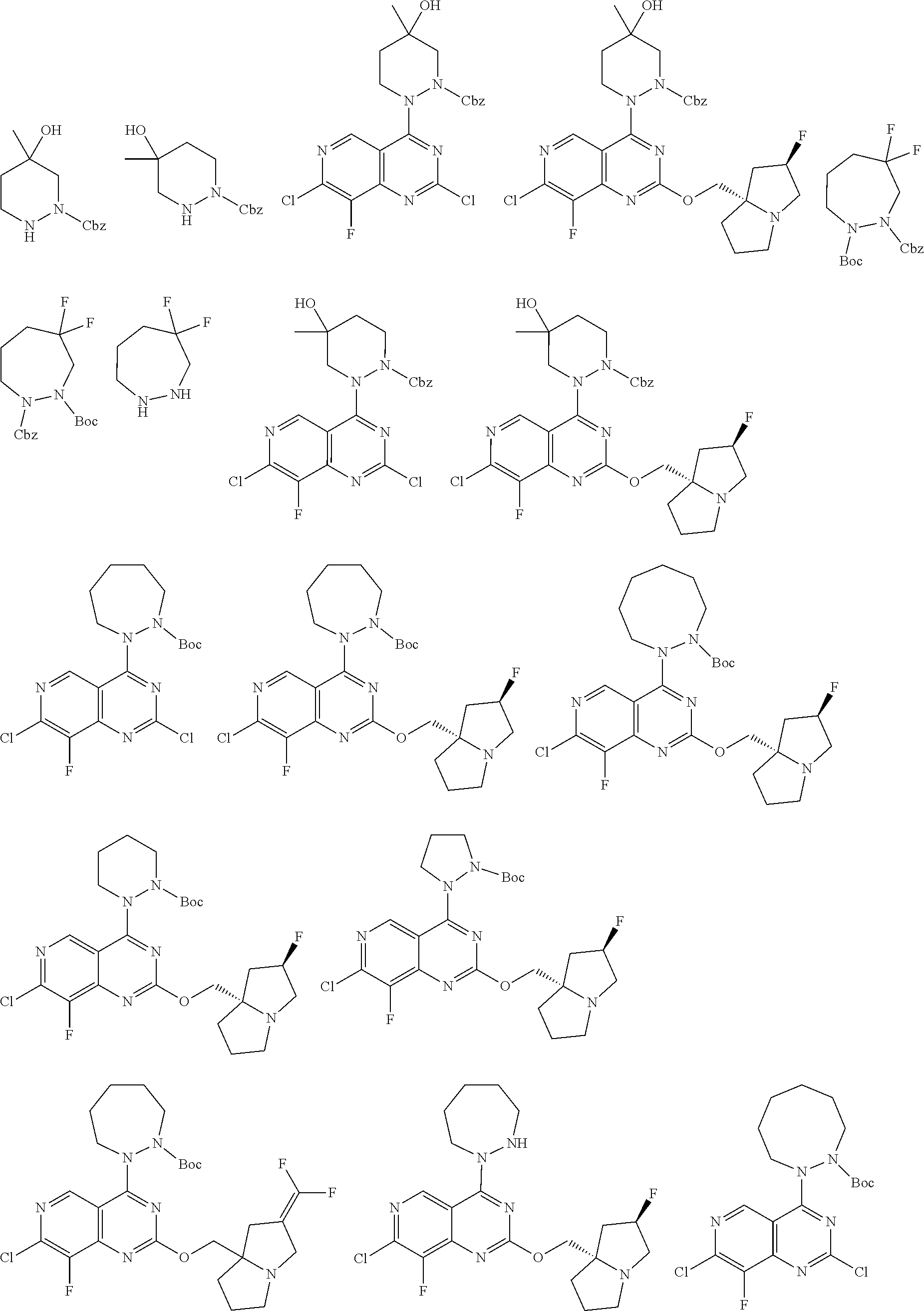

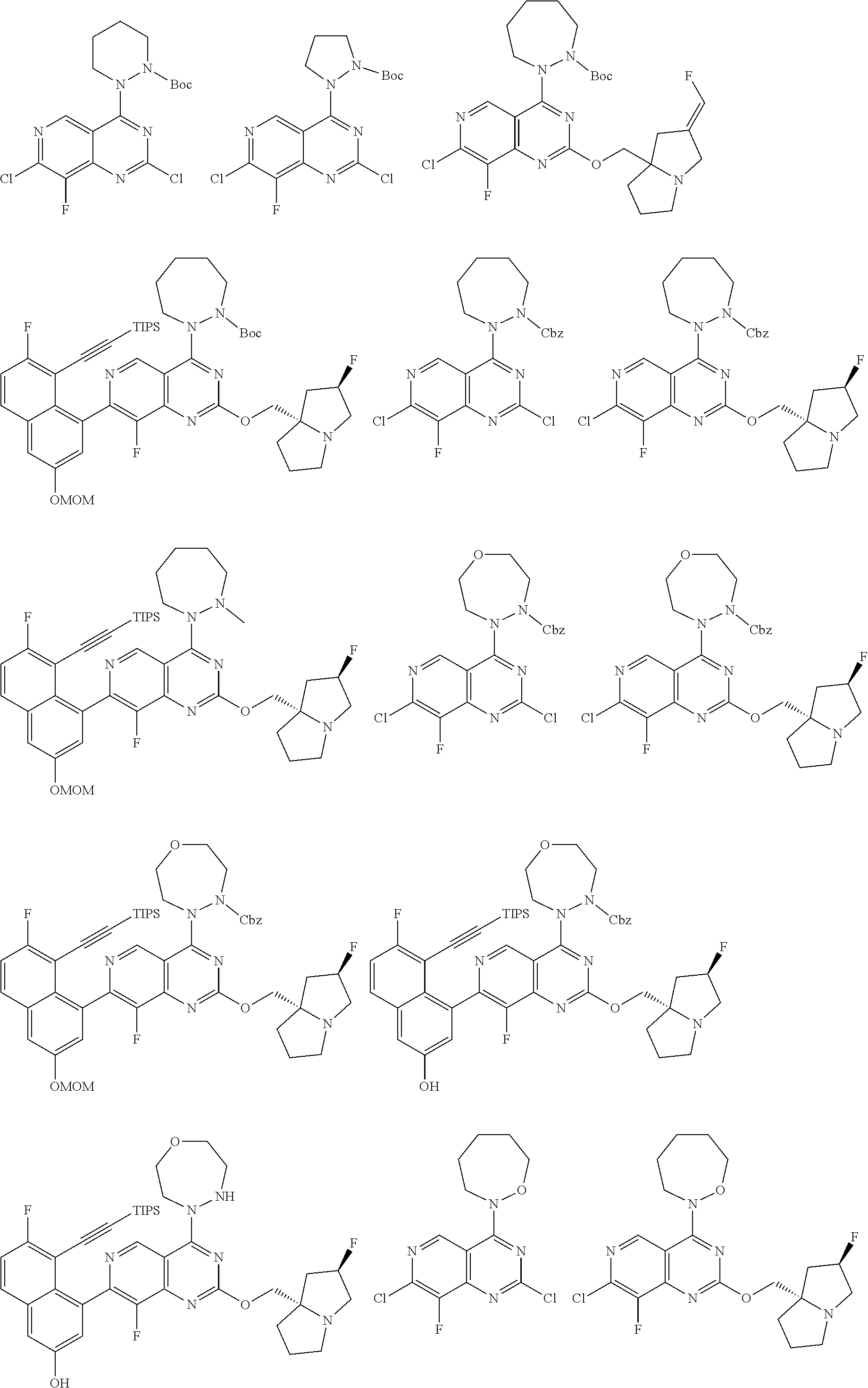

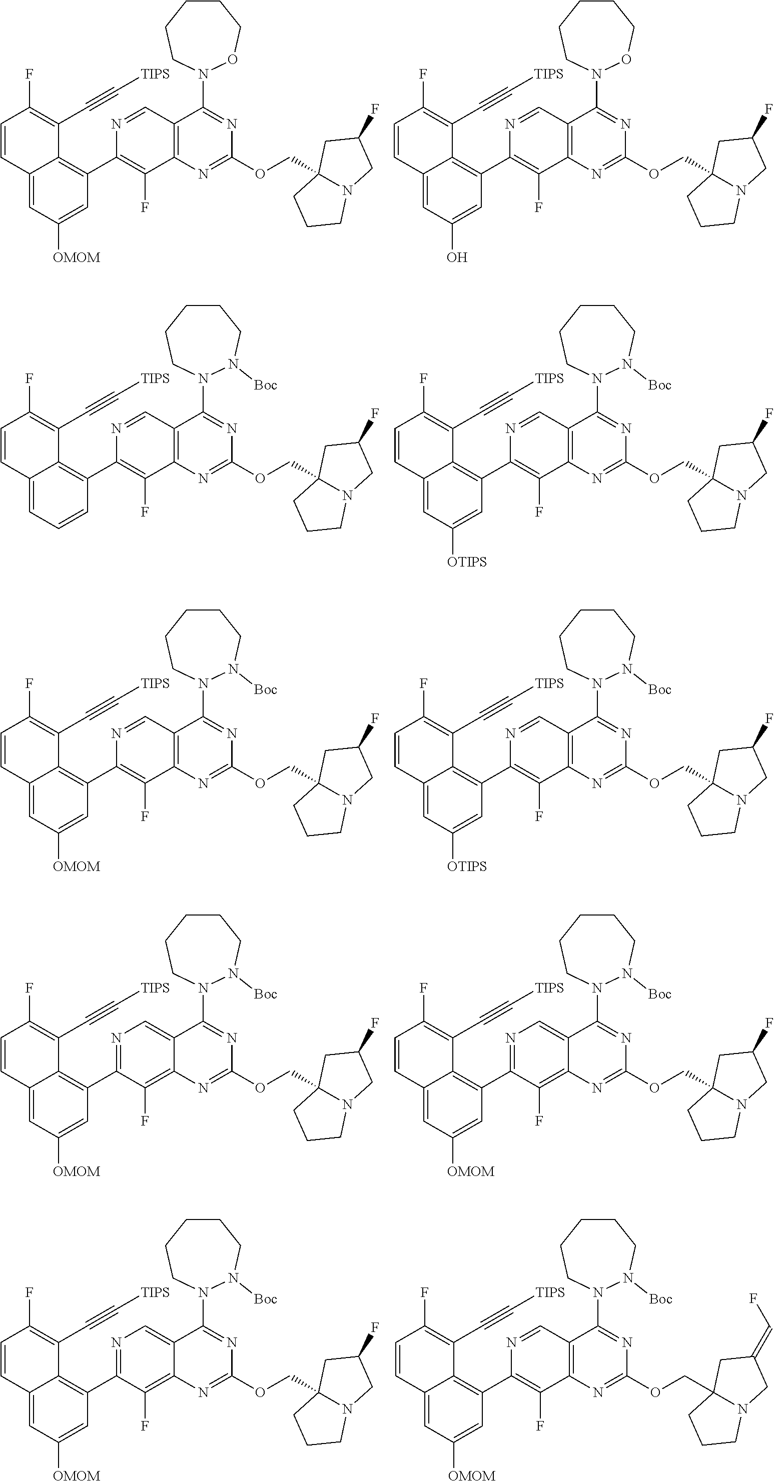

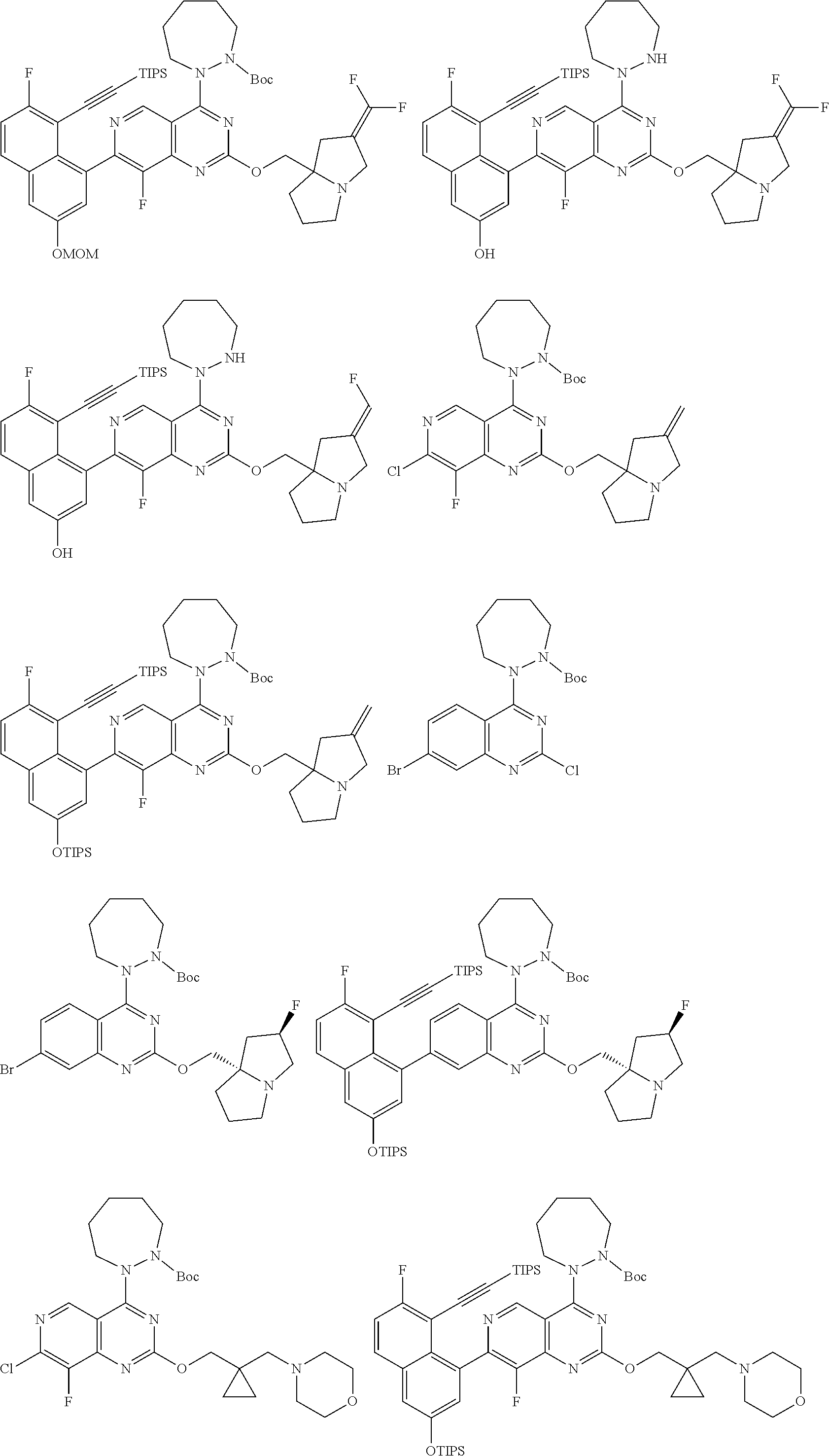

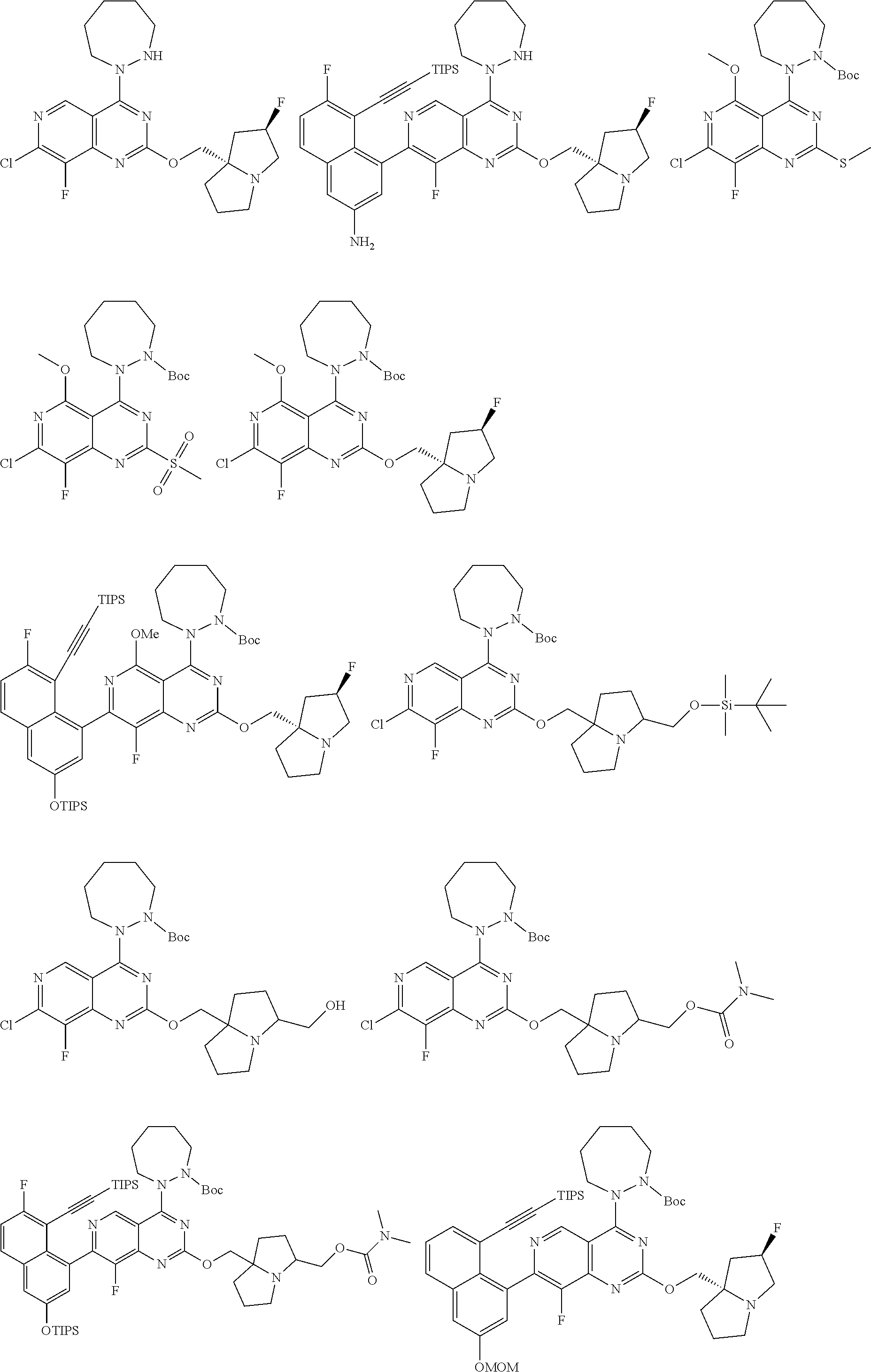

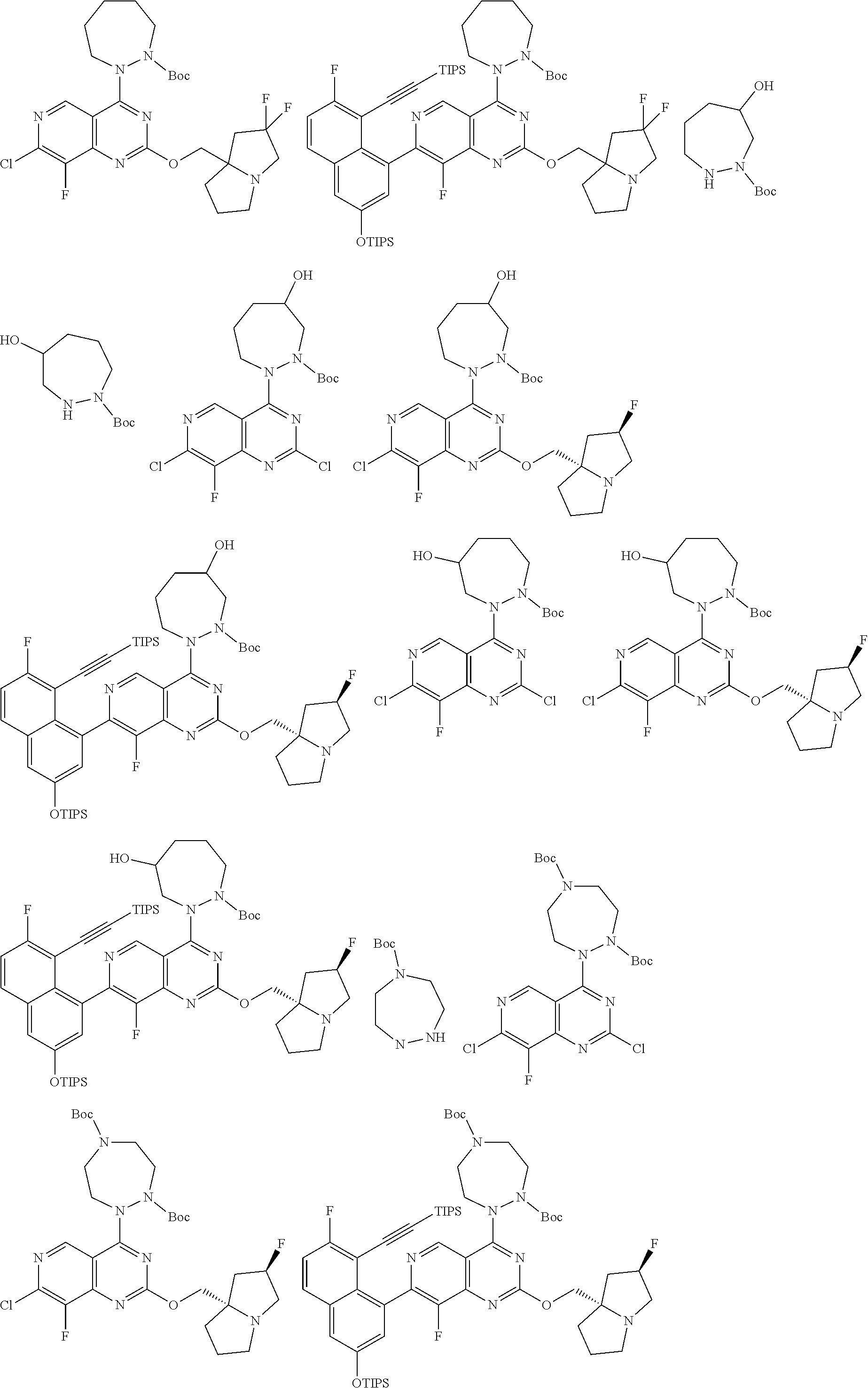

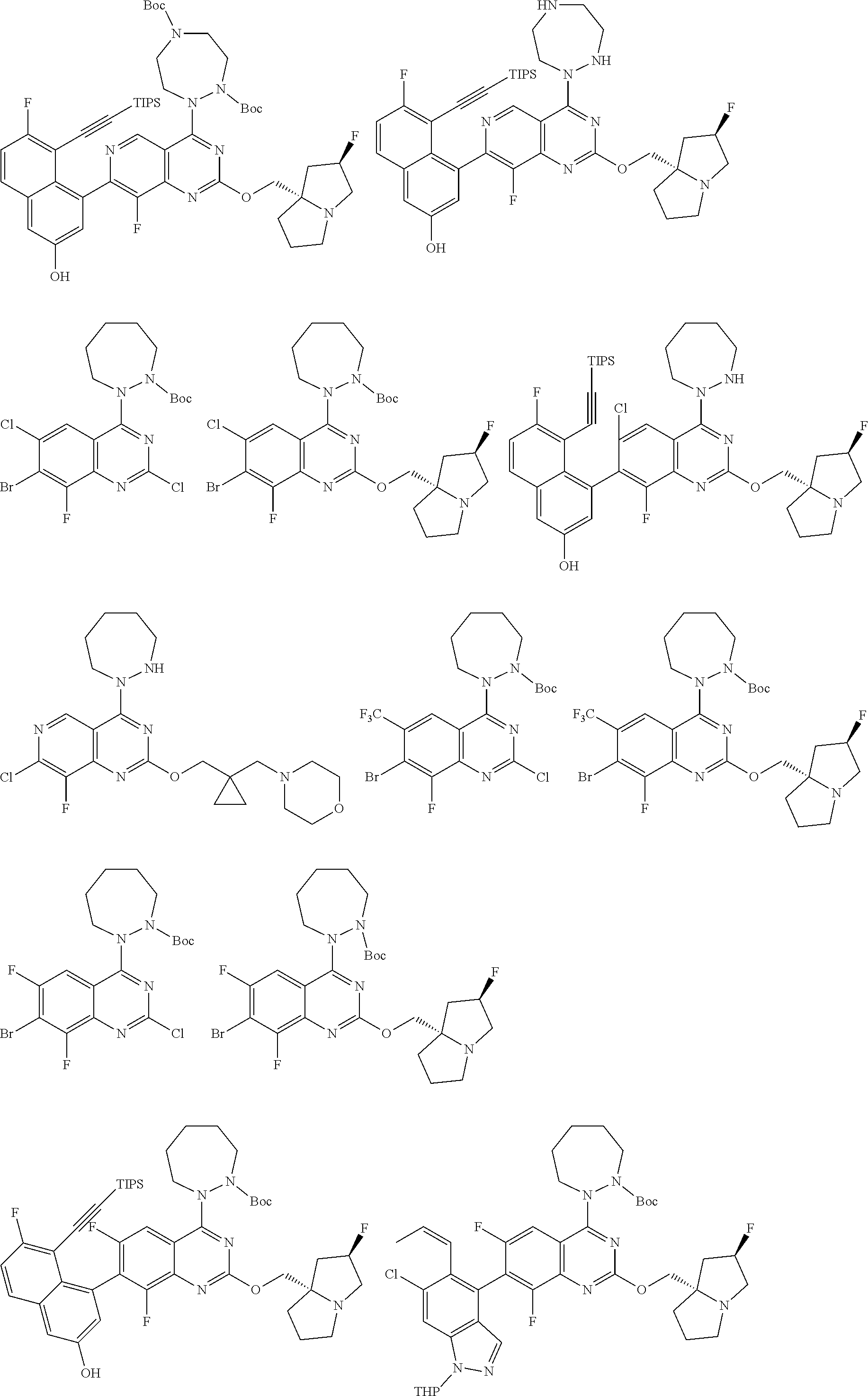

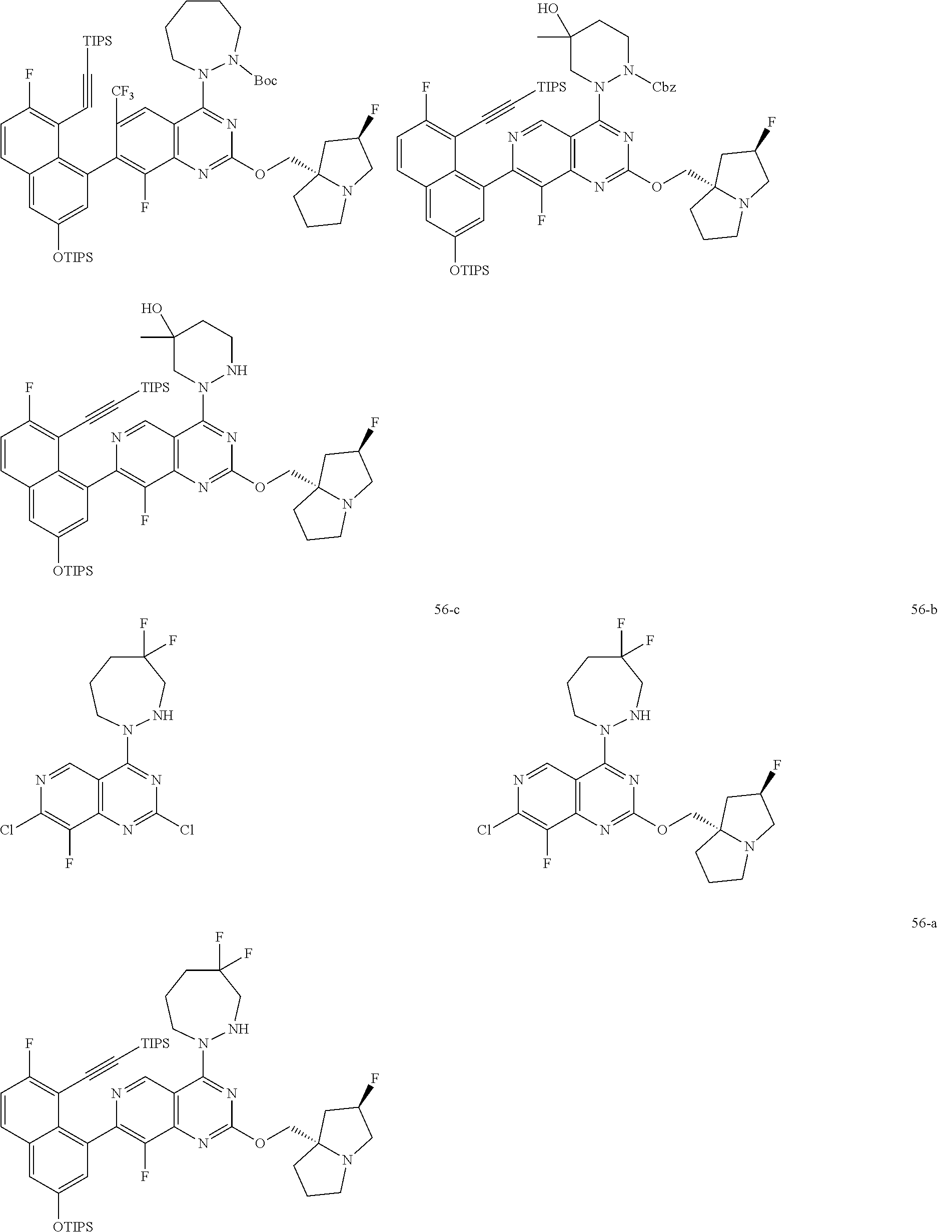

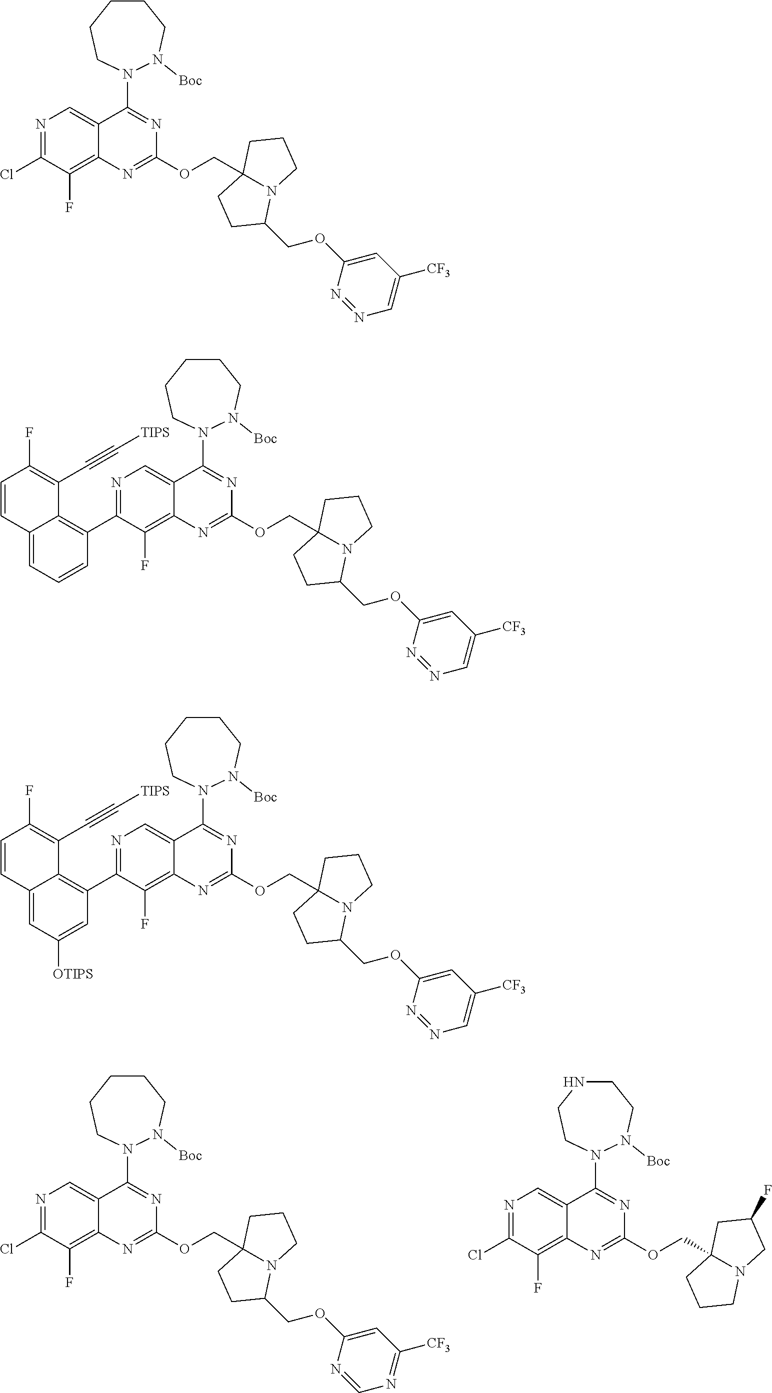

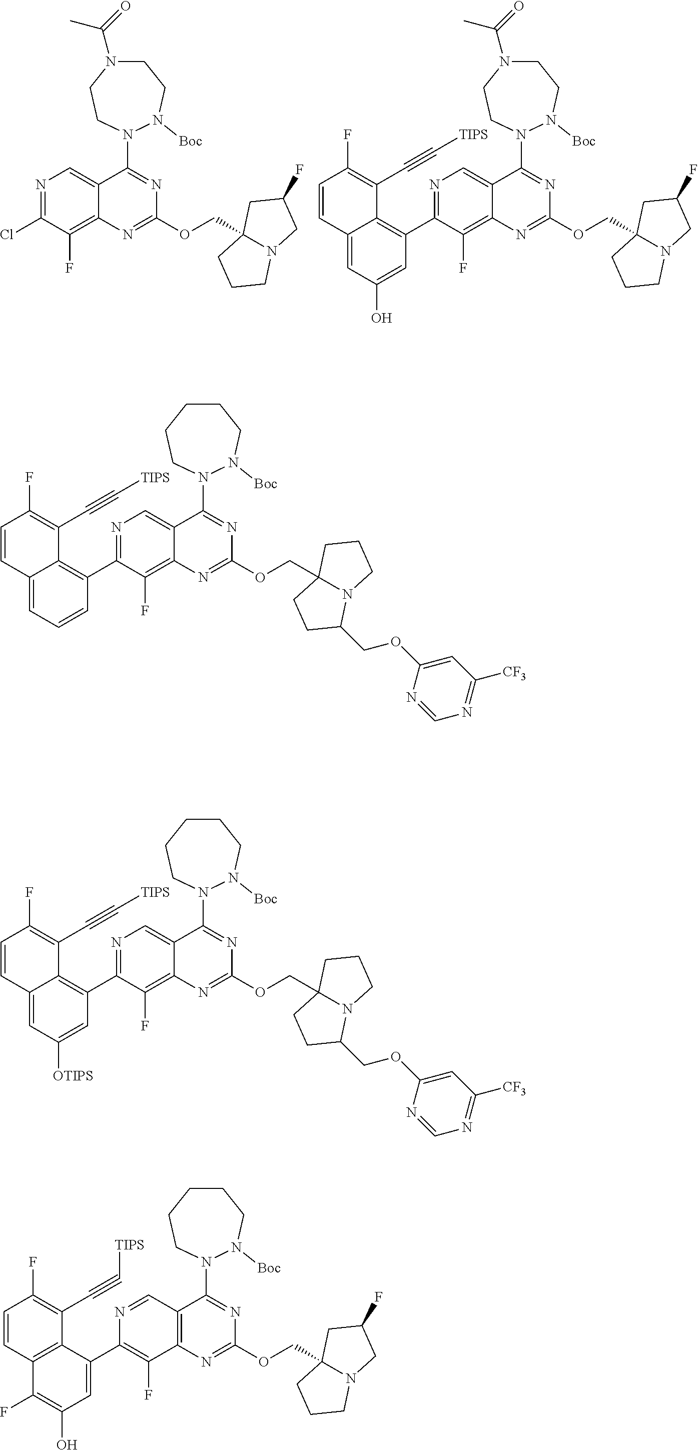

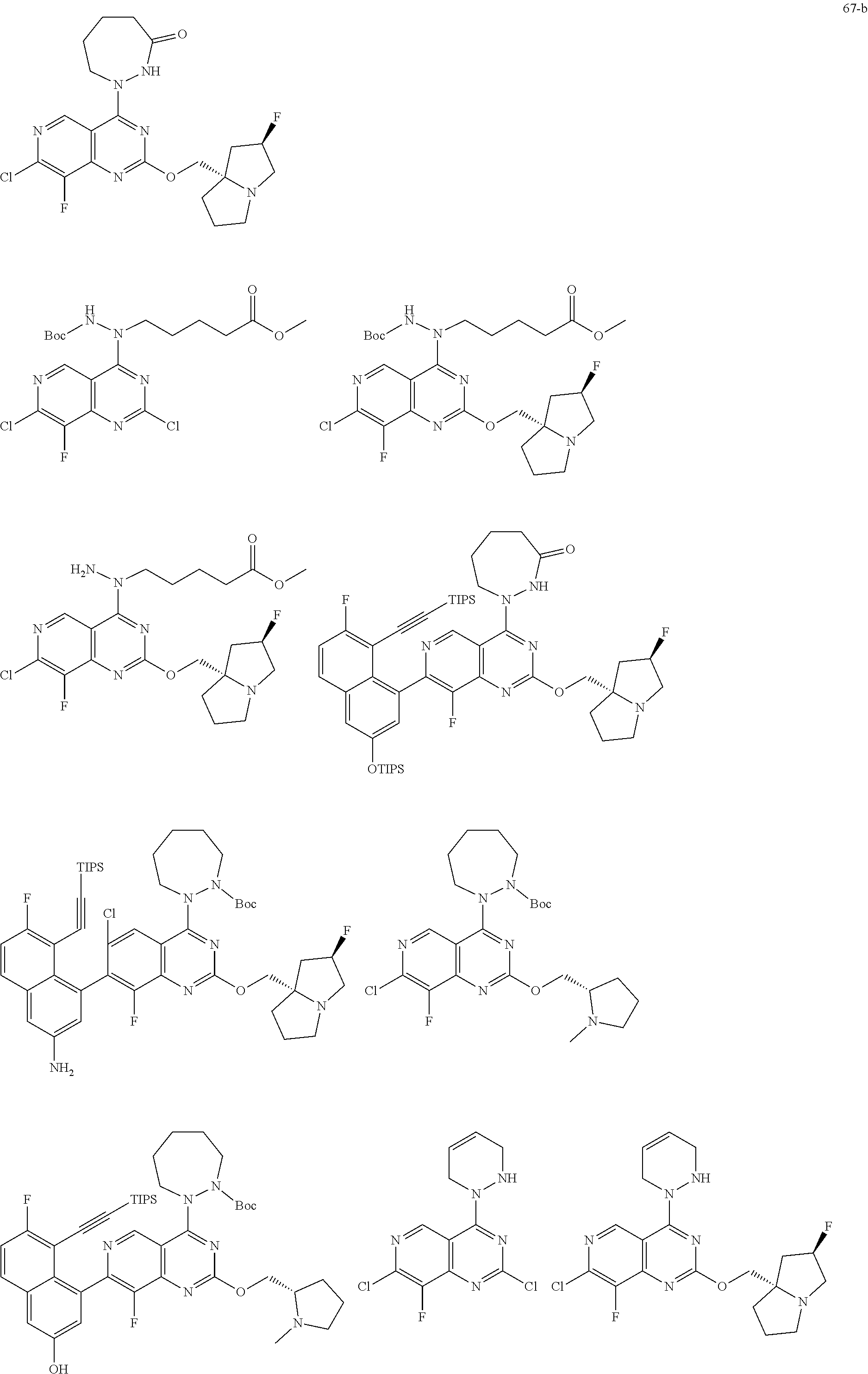

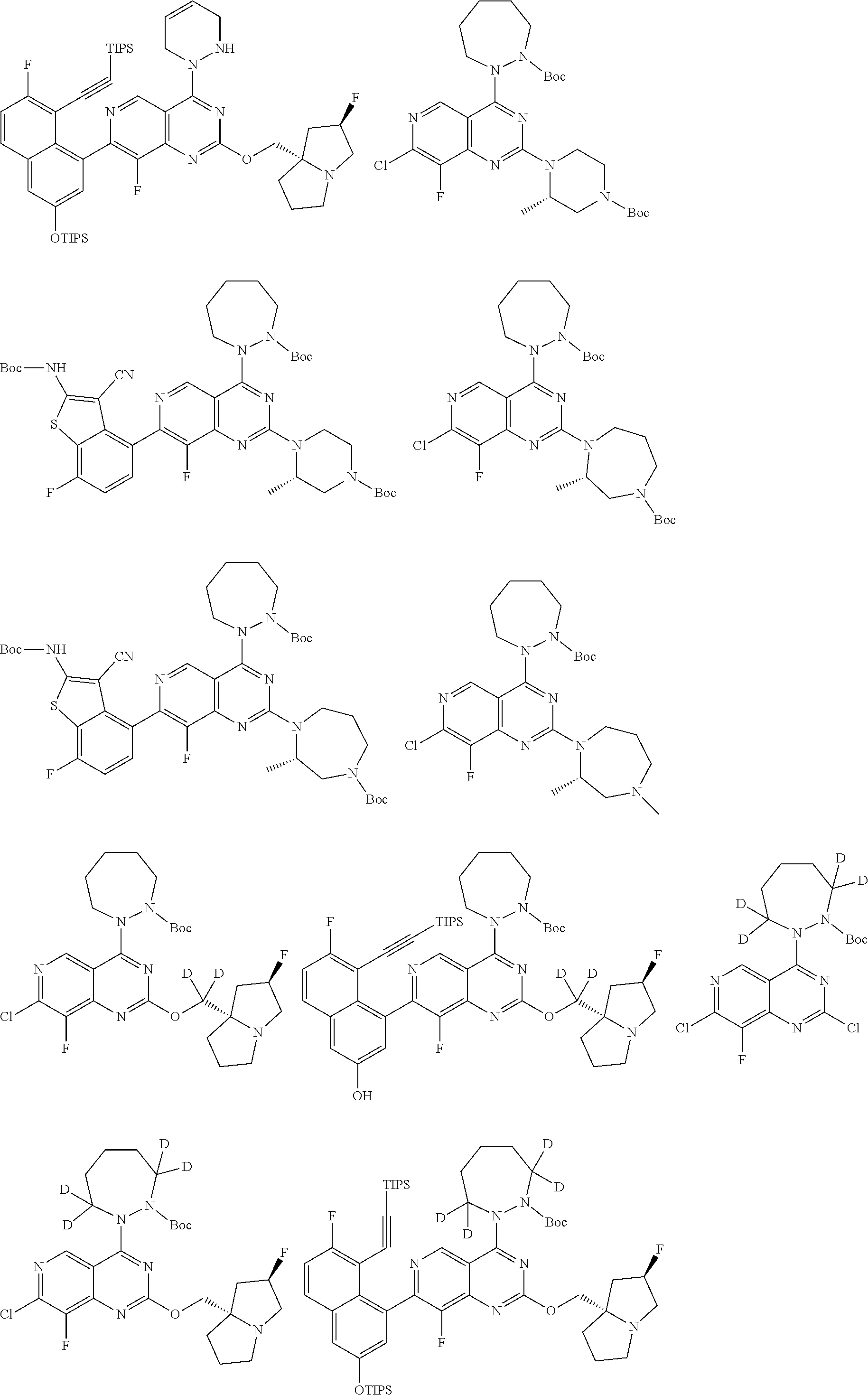

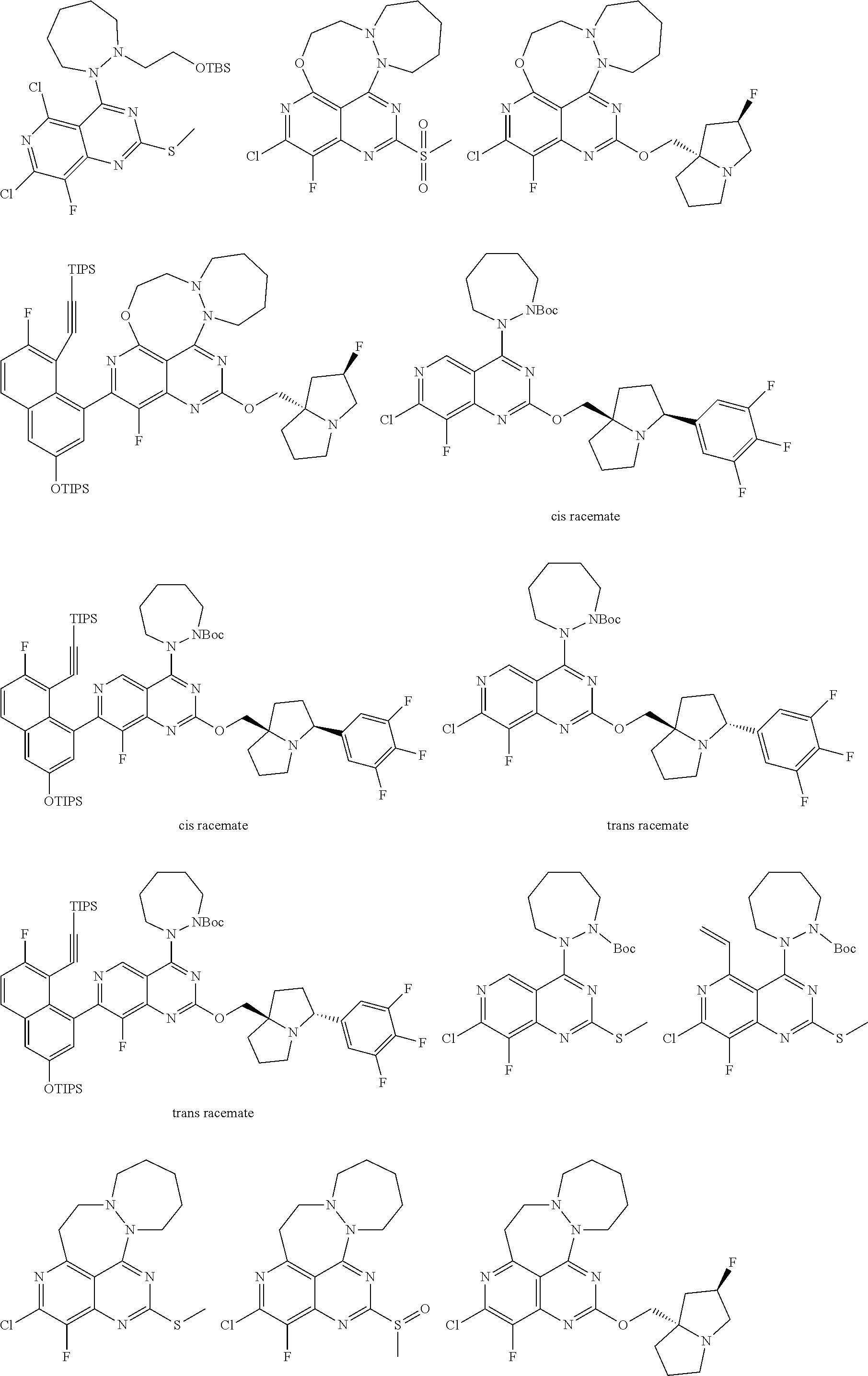

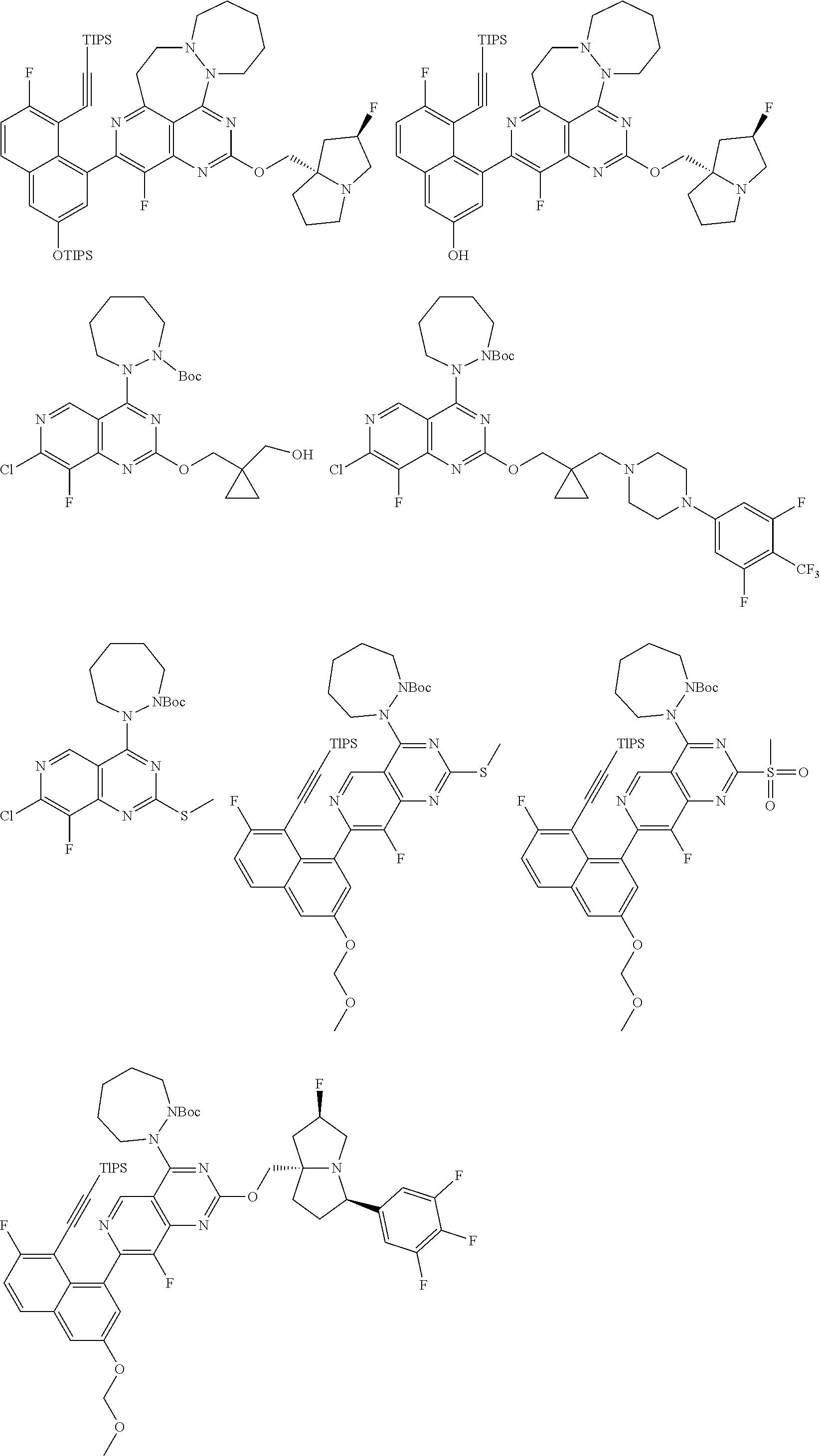

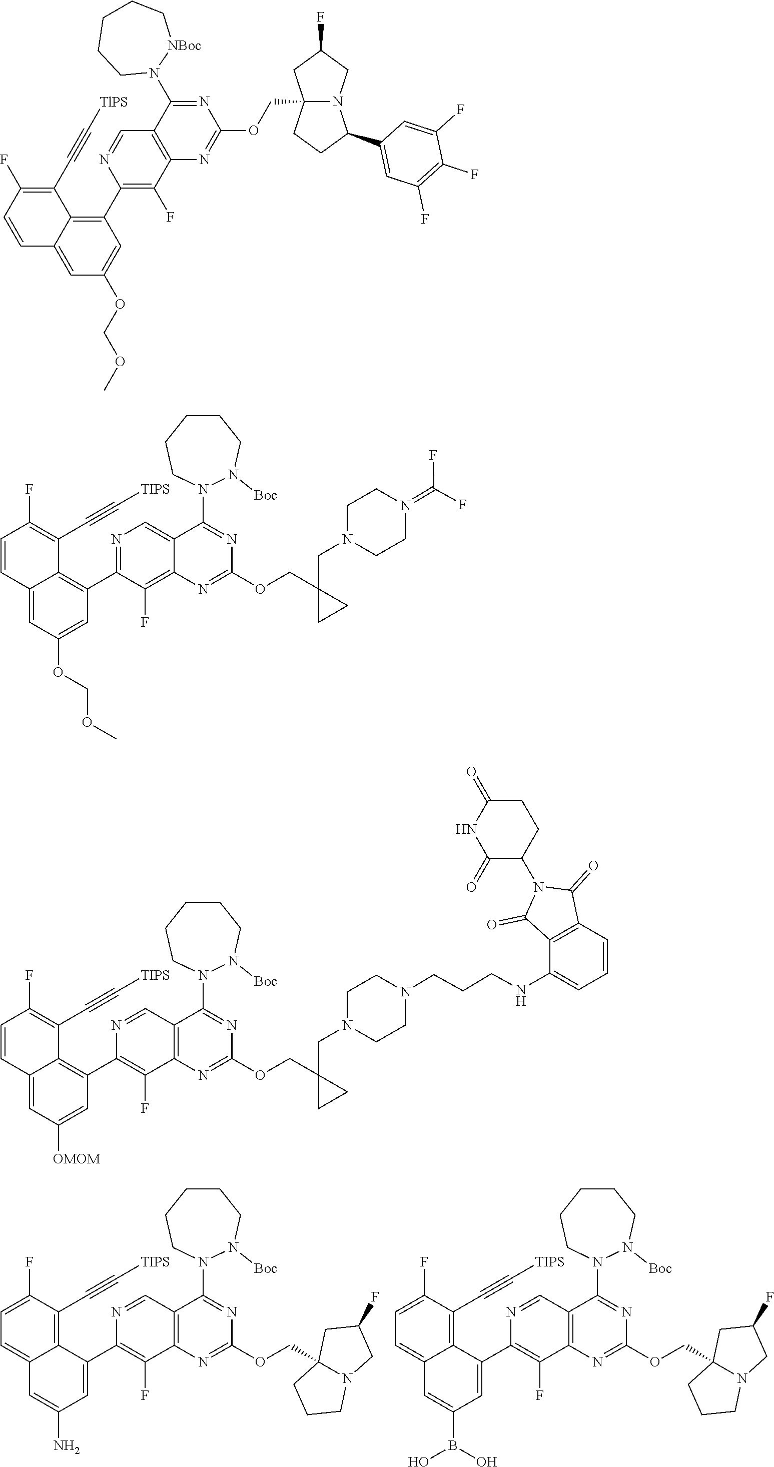

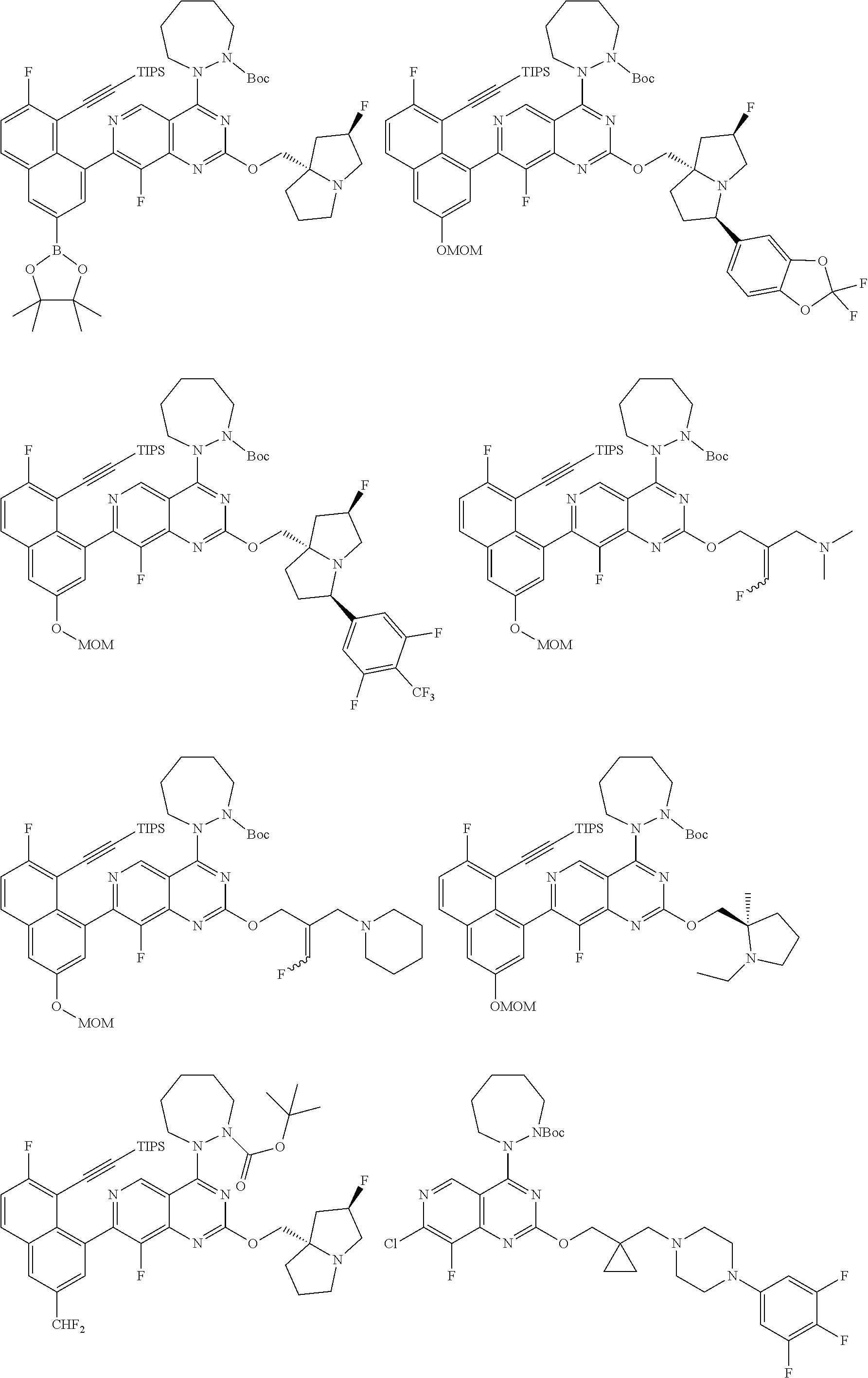

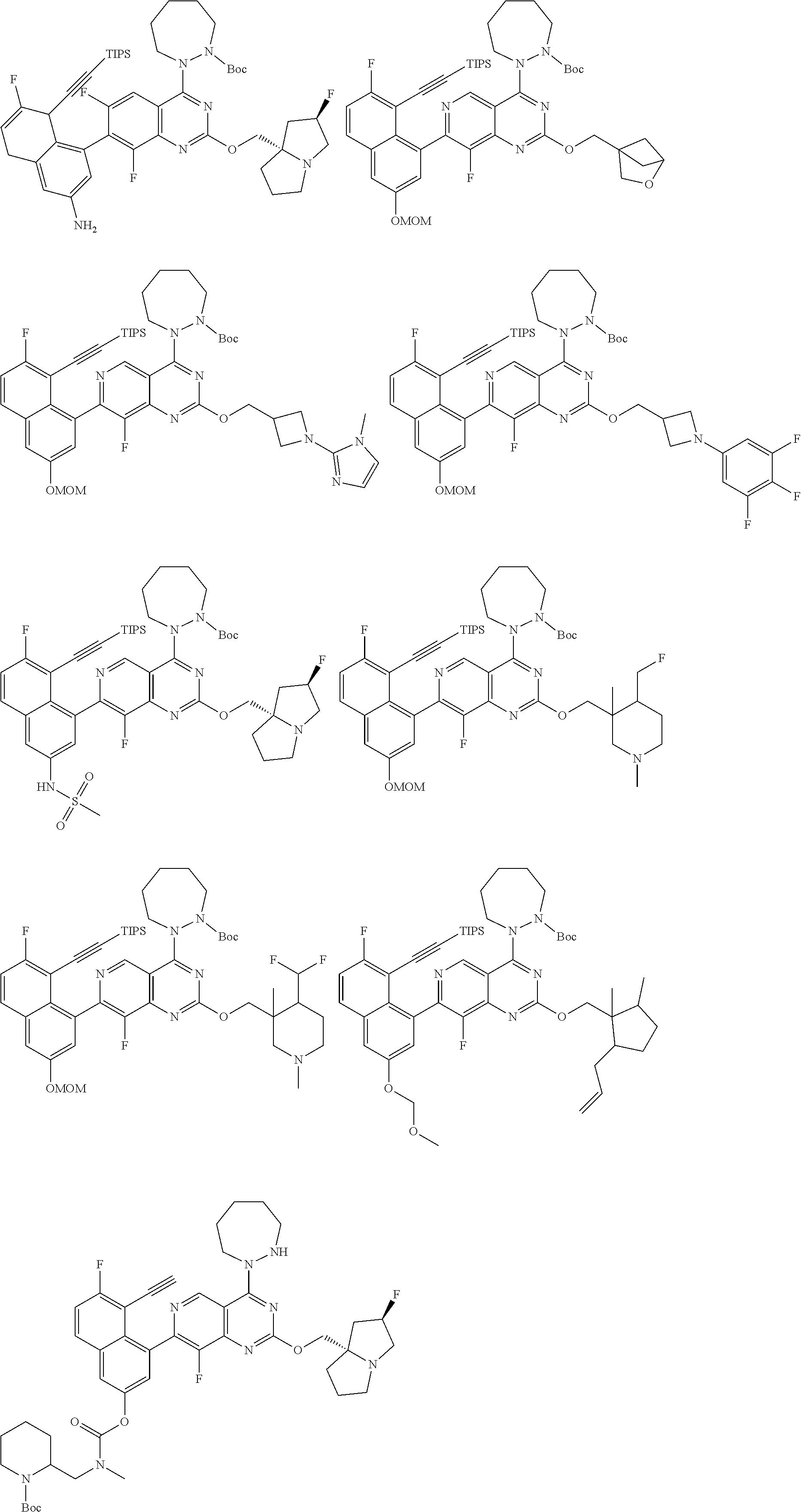

The present disclosure also provides a pharmaceutical composition comprising a substance A and a pharmaceutically acceptable excipient, wherein the substance A is the compound containing structure of a nitrogen-containing heterocyclic compound of formula I, the pharmaceutically acceptable salt thereof, the prodrug thereof, the stereoisomer thereof, the tautomer thereof or the isotopically labeled compound thereof as described above.

The present disclosure also provides a method for inhibiting RAS in a subject in need thereof, comprising: administering a therapeutically effective amount of a substance A to the subject, wherein the substance A is the compound containing structure of a nitrogen-containing heterocyclic compound of formula I, the pharmaceutically acceptable salt thereof, the prodrug thereof, the stereoisomer thereof, the tautomer thereof or the isotopically labeled compound thereof as described above.

In the method for inhibiting RAS in a subject in need thereof, wherein the RAS is wild type RAS or mutated forms of RAS. The wild type RAS is wild-amplified KRAS. The mutated forms of RAS such as a KRAS, HRAS or NRAS mutation, wherein the mutated forms of KRAS can be mutated G12, G13 or Q61, for example, KRAS G12C, KRAS G12D, KRAS G12S, KRAS G12A, KRAS G12V or KRAS G13D, and for another example, KRAS G12C, KRAS G12D or KRAS G12V; the mutated forms of HRAS be mutated G12, G13 or Q61, for example, mutated HRAS G12C, HRAS G12D, HRAS G12S, HRAS G12A, HRAS G12V or HRAS G13D; the mutated forms of NRAS can be mutated G12, G13 or Q61, for example, NRAS G12C, NRAS G12D, NRAS G12S, NRAS G12A, NRAS G12V or NRAS G13D.

The present disclosure also provides a method for treating or preventing a RAS-related disease in a subject in need thereof, comprising: administering a therapeutically effective amount of a substance A to the subject, wherein the substance A is the compound containing structure of the nitrogen-containing heterocyclic compound of formula I, the pharmaceutically acceptable salt thereof, the prodrug thereof, the stereoisomer thereof, the tautomer thereof or the isotopically labeled compound thereof as described above.

In the method for inhibiting RAS in a subject in need thereof, wherein the RAS is wild type RAS or mutated forms of RAS. The wild type RAS is wild-amplified KRAS. The mutated forms of RAS such as a KRAS, HRAS or NRAS mutation, wherein the mutated forms of KRAS can be mutated G12, G13 or Q61, for example, KRAS G12C, KRAS G12D, KRAS G12S, KRAS G12A, KRAS G12V or KRAS G13D, and for another example, KRAS G12C, KRAS G12D or KRAS G12V; the mutated forms of HRAS be mutated G12, G13 or Q61, for example, mutated HRAS G12C, HRAS G12D, HRAS G12S, HRAS G12A, HRAS G12V or HRAS G13D; the mutated forms of NRAS can be mutated G12, G13 or Q61, for example, NRAS G12C, NRAS G12D, NRAS G12S, NRAS G12A, NRAS G12V or NRAS G13D.

In the method for treating or preventing a RAS-related disease in a subject in need thereof, wherein the RAS-related disease is cancer, the cancer is selected from the group consisting of colon cancer, appendiceal cancer, pancreatic cancer, MYH-related polyposis, hematologic cancer, breast cancer, endometrial cancer, gallbladder cancer, bile duct cancer, prostate cancer, lung cancer, brain cancer, ovarian cancer, cervical cancer, testicular cancer, kidney cancer, head or neck cancer, bone cancer, skin cancer, rectal cancer, liver cancer, esophageal cancer, stomach cancer, thyroid cancer, bladder cancer, lymphoma, leukemia and melanoma.

The present disclosure also provides a method for treating or preventing cancer in a subject in need thereof, comprising: administering a therapeutically effective amount of a substance A to the subject, wherein the substance A is the compound containing structure of the nitrogen-containing heterocyclic compound of formula I, the pharmaceutically acceptable salt thereof, the prodrug thereof, the stereoisomer thereof, the tautomer thereof or the isotopically labeled compound thereof as described above; the cancer is selected from the group consisting of colon cancer, appendiceal cancer, pancreatic cancer, MYH-related polyposis, hematologic cancer, breast cancer, endometrial cancer, gallbladder cancer, bile duct cancer, prostate cancer, lung cancer, brain cancer, ovarian cancer, cervical cancer, testicular cancer, kidney cancer, head or neck cancer, bone cancer, skin cancer, rectal cancer, liver cancer, esophageal cancer, stomach cancer, thyroid cancer, bladder cancer, lymphoma, leukemia and melanoma.

The term “more” refers to 2, 3, 4 or 5.

The term “pharmaceutically acceptable salt” refers to a salt of the compound disclosed herein which is prepared using relatively safe and pharmaceutically acceptable acids or bases. When the compound disclosed herein contains a relatively acidic functional group, a base addition salt can be obtained by contacting a neutral form of the compound with a sufficient amount of a pharmaceutically acceptable base in a pure solution or a suitable inert solvent. Pharmaceutically acceptable base addition salts include, but are not limited to: lithium salt, sodium salt, potassium salt, calcium salt, aluminum salt, magnesium salt, zinc salt, bismuth salt, ammonium salt, and diethanolamine salt. When the compound disclosed herein contains a relatively basic functional group, an acid addition salt can be obtained by contacting a neutral form of the compound with a sufficient amount of a pharmaceutically acceptable acid in a pure solution or a suitable inert solvent. The pharmaceutically acceptable acid includes inorganic acids, including, but not limited to: hydrochloric acid, hydrobromic acid, hydroiodic acid, nitric acid, carbonic acid, phosphoric acid, phosphorous acid, and sulfuric acid. The pharmaceutically acceptable acids include organic acids, including, but not limited to: acetic acid, propionic acid, oxalic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, salicylic acid, tartaric acid, methanesulfonic acid, isonicotinic acid, acidic citric acid, oleic acid, tannic acid, pantothenic acid, bitartrate, ascorbic acid, gentisic acid, fumaric acid, gluconic acid, saccharic acid, formic acid, ethanesulfonic acid, pamoic acid (i.e., 4,4′-methylene-bis(3-hydroxyl-2-naphthoic acid)), and amino acids (e.g., glutamic acid and arginine). When a compound disclosed herein contains both relatively acidic functional group and relatively basic functional group, it can be converted to either base addition salt or acid addition salt. For details, see Berge et al., “Pharmaceutical Salts”, Journal of Pharmaceutical Science 66:1-19 (1977), or Handbook of Pharmaceutical Salts: Properties, Selection, and Use (P. Heinrich Stahl and Camille G. Wermuth, ed., Wiley-VCH, 2002).

The term “stereoisomer” refers to isomers of a molecule having the same order of atoms or radicals but different spatial arrangement, such as cis-trans isomer, optical isomer or atropisomer. Such stereoisomers can be separated, purified and enriched by asymmetric synthesis or chiral resolution (including but not limited to thin layer chromatography, centrifugal partition chromatography, column chromatography, gas chromatography, and high pressure liquid chromatography), and can also be obtained by chiral resolution via bonding (chemical bonding, etc.) or salification (physical bonding, etc.) with other chiral compounds.

The term “tautomer” refers to functional isomers of a molecule where an atom rapidly migrates between two positions. For example, acetone and 1-propen-2-ol can be mutually converted when hydrogen atom rapidly migrates between oxygen and α-carbon atoms.

The term “isotopically labeled compound” refers to a compound where one or more atoms are replaced by one or more atoms having a specific atomic mass or mass number. Examples of isotope that can be incorporated into the compound disclosed herein include, but are not limited to, isotopes of hydrogen, carbon, nitrogen, oxygen, fluorine, sulfur, and chlorine (e.g., 2H, 3H, 13C, 14C, 15N, 18O, 17O, 18F, 35S, and 36C₁). An isotopically labeled compound of the present disclosure can generally be prepared by replacing a non-isotopically labeled reagent with an isotopically labeled reagent according to the methods described herein.

The term “halogen” refers to fluorine, chlorine, bromine or iodine.

The term “alkyl” refers to a linear or branched alkyl group having a specified number of carbon atoms. Examples of alkyl include methyl, ethyl, n-propyl, isopropyl, n-butyl, tert-butyl, isobutyl, sec-butyl, n-pentyl, n-hexyl, n-heptyl, n-octyl, and the like.

The term “alkylene” refers to a linking group between two other species, which may be linear or branched. Examples include, but are not limited to, —CH₂—, —CH₂CH₂—, —CH₂CH₂CH₂CH(CH)— and —CH₂CH(CH₂CH₃)CH₂—.

The term “alkoxy” refers to the group —O—R^{x}, wherein RX is an alkyl as defined above.

The terms “cycloalkyl” and “carbocyclic ring” refer to a saturated cyclic group consisting only of carbon atoms having a specified number of carbon atoms (e.g., C₃-C₆), which is a monocyclic, bridged or spiro ring. The cycloalkyl includes, but is not limited to, cyclopropyl, cyclobutyl, cyclopentyl, and cyclohexyl.

The term “aryl” refers to an aromatic group consisting of carbon atoms, each ring having aromaticity. For example, phenyl or naphthyl.

The term “heteroaryl” refers to a cyclic group having a specified number of ring atoms (e.g., 5-12 members), a specified number of heteroatoms (e.g., 1, 2, or 3) and specified heteroatom species (one or more of N, O and S), which is monocyclic or polycyclic, and has at least one aromatic ring (according to the Hückel's rule). Heteroaryls are linked to other fragments of the molecule through aromatic or non-aromatic rings. Heteroaryls include, but are not limited to, furyl, pyrrolyl, thienyl, pyrazolyl, imidazolyl, oxazolyl, thiazolyl, pyridinyl, pyrimidinyl, and indolyl.

The terms “heterocyclyl”, “heterocycle” or “heterocycloalkyl” refer to a cyclic group having a specified number of ring atoms (e.g., 3-8 members), a specified number of heteroatoms (e.g., 1, 2, or 3) and specified heteroatom species (one or more of N, O and S), which is monocyclic, bridged, or spiro, and where each ring is saturated. Heterocycloalkyls include, but are not limited to, azetidinyl, tetrahydropyrrolyl, tetrahydrofuryl, morpholinyl, piperidinyl, and the like.

The term “hydroxyl” refers to a —OH group.

The term “cyano” refers to a —CN group.

The term “oxo” refers to a ═O group.

Unless otherwise specified,

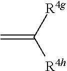

is referred as two hydrogens on the same carbon are substituted with, for example, when the two hydrogens are substituted with

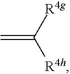

forming a structure of

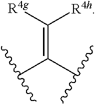

The same is for definition of

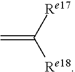

Substituted “Cₓ₁-C_{y1}” groups with specified numbers of carbon atoms (x1 and y1 are integers), for example, “Cₓ₁-C_{y1}” alkyl, “Cₓ₁-C_{y1}” cycloalkyl, “Cₓ₁-C_{y1}” cycloalkenyl, “Cₓ₁-C_{y1}” alkoxyl, “Cₓ₁-C_{y1}” alkenyl, “Cₓ₁-C_{y1}” alkynyl, “Cₓ₁-C_{y1}” aryl, “Cₓ₁-C_{y1}” heteroaryl, or “Cₓ₁-C_{y1}” heterocyclyl, all represent numbers of carbon atoms excluding substituents, e.g., a C₁-C₆ alkyl represents a C₁-C₆ alkyl excluding substituents.

In the present disclosure, when R¹² and R¹³, R¹⁵ and R¹⁶, R^{c3} and R^{c4}, R²² and R²³, R²⁵ and R²⁶, R^{4b1} and R^{4b2}, R^{4d1} and R^{4d2}, R^{4f1} and R^{4f2}, R⁴ⁱ and R^{4j}, R^{e1} and R^{e2}, R^{e3} and R^{e4}, R^{e5} and R^{e6}, R^{e7} and R^{e8}, R^{e10} and R^{e11}, R^{e13} and R^{e14}, R^{e15} and R^{e16}, R^{e173} and R^{e174} together with the atom N to which they are attached form “4- to 12-membered heterocycloalkyl containing 1-3 heteroatoms, of which one is N, the other independently selected from O, S and N”, or, “4- to 12-membered heterocycloalkyl containing 1-3 heteroatoms, of which one is N, the other independently selected from O, S and N” substituted with one or more R³1-³; the combination of heteroatoms in the “4- to 12-membered heterocycloalkyl containing 1-3 heteroatoms, of which one is N, the other independently selected from O, S and N” and “4- to 12-membered heterocycloalkyl containing 1-3 heteroatoms, of which one is N, the other independently selected from O, S and N” substituted with one or more R³⁻¹⁻³ is (N), (N and O), (N and S) or (N, O and S).

The above preferred conditions may be combined arbitrarily to obtain preferred embodiments of the present disclosure without departing from the general knowledge in the art.

The reagents and starting materials used in the present disclosure are commercially available.

The positive/progressive effects of the present disclosure are as follows: The nitrogen-containing heterocyclic compounds as represented in Formula I in the present invention have good inhibitory effects on cells of various mutations such as KRAS G12C, KRAS G12D, KRAS G12V, KRAS Q61H and other mutations, as well as on KRAS-amplified wild-type cells, and are expected to treat and/or prevent a wide range of diseases associated with Ras.

### DETAILED DESCRIPTION OF THE EMBODIMENTS

The present disclosure is further illustrated by the following examples, which are not intended to limit the present disclosure. Experimental procedures without specified conditions in the following examples were performed in accordance with conventional procedures and conditions, or in accordance with instructions.

The solvents involved in the following embodiment are all analytical or chromatographic pure. In the examples below, the ratio of solvents is by volume, if not otherwise specified.

In the present disclosure, room temperature refers to ambient temperature, or 10-35° C. Overnight refers to 8-15 hours. Reflux refers to the reflux temperature of a solvent at atmospheric pressure.

The following is a list of abbreviations used in the examples:
DMF N, N-dimethylformamide
DMAc N, N-dimethylacetamide
DIPEA diisopropylethylamine
TIPS Triisopropylsilyl
THF Tetrahydrofuran
MOM Methoxymethyl
DCM dichloromethane
EA ethyl acetate
DMSO dimethyl sulfoxide
PE petrol ether
MeOH methanol
cataCXium A Pd G3 [n-Butylbis(1-adamantyl)phosphine](2-amino-1,1′-biphenyl-2-yl)palladium methanesulfonate (II)
Pd(DPEphos)Cl₂ Bis(diphenylphosphine phenyl ether)palladium(II) chloride
PdCl₂(dtbpf) Dichloro[1,1′-bis(di-tert-butylphosphino)ferrocene]palladium (II)
DABCO 1,4-Diazabicyclo[2.2.2]octane
Pd(dppf)Cl₂ [1,1′-bis(diphenylphosphino)ferrocene]palladium(II) dichloride
Pd₂(dba)₃ tris(dibenzylideneacetone)dipalladium
TFA Trifluoroacetic acid
Boc tert-butoxycarbonyl
Cbz benzyloxycarbonyl
NBS N-Bromosuccinimide
TMSOTf Trimethylsilyl trifluoromethanesulfonate
TBSOTf tert-Butyldimethylsilyl trifluoromethanesulfonate

### Example 1 Synthetic Route of Compound 1

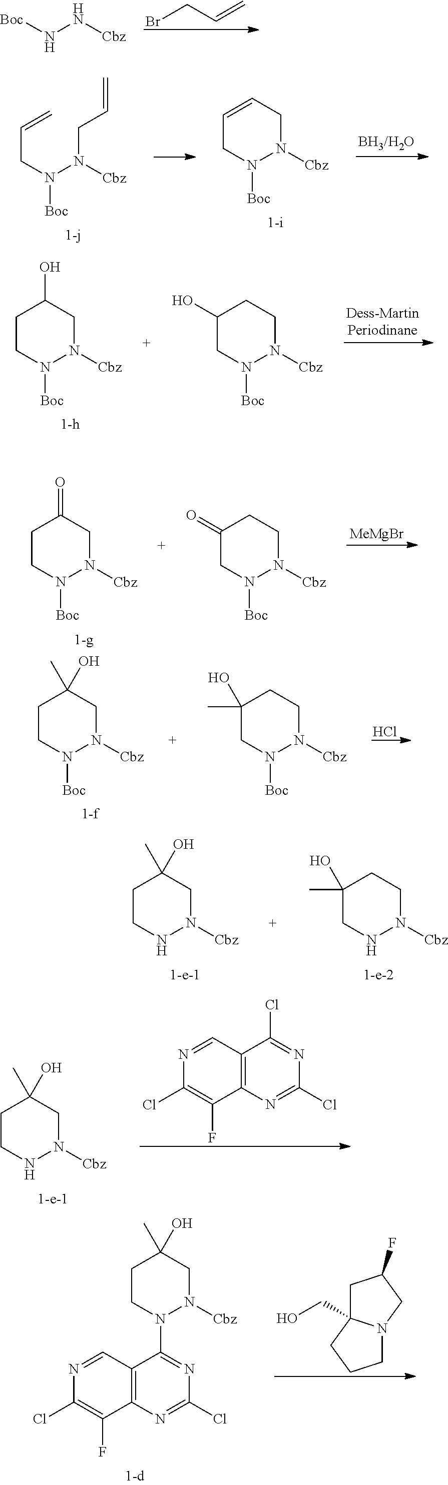

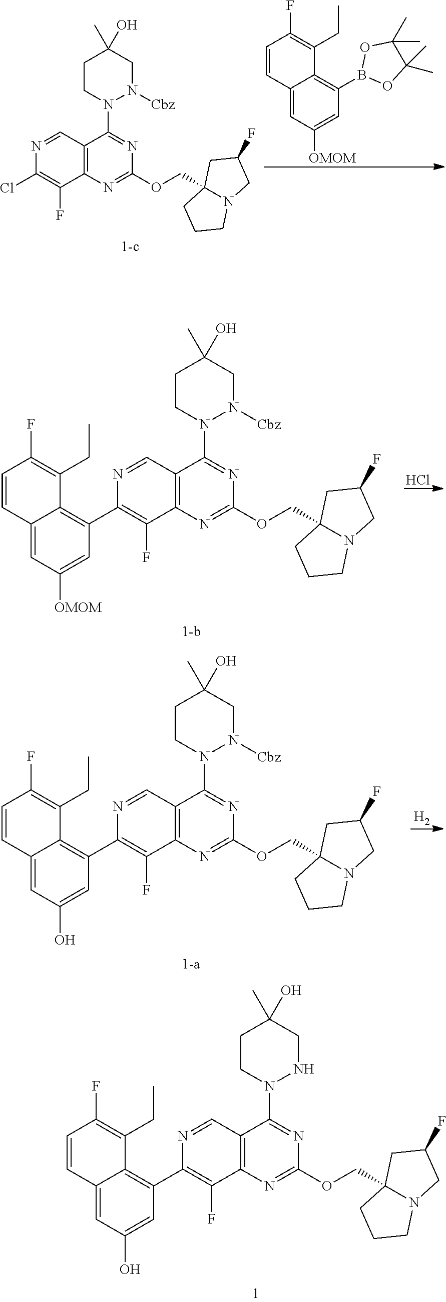

### Synthesis of Compound 1-j

To a reaction vial were added 2-(tert-butyl)hydrazine-1,2-dicarboxylic acid-1-benzyl ester (1000 mg, 3.76 mmol) and DMF (10 mL) at room temperature. After degassed and purged with nitrogen, and cooled in an ice-water bath under protection of nitrogen, the above mixture was added NaH (60%, 376 mg, 9.40 mmol) in batches. After stirred for 5 min after addition, the resulting mixture was added 3-bromopropene (1000 mg, 8.27 mmol) slowly dropwise over half an hour. After completion of the addition, the reaction mixture was warmed to room temperature naturally and stirred for another 3 hours. The reaction mixture was added 100 mL of water and 100 mL of ethyl acetate, partitioned and the aqueous phase was extracted with ethyl acetate. The organic phases were combined, washed with water, dried and concentrated to obtain compound 1-j (1200 mg, 92%). LC-MS (ESI): m/z 247.2 (M−Boc+H)⁺.

### Synthesis of Compound 1-i

To a reaction vial were added 1-j (1200 mg, 3.46 mmol), DCM (180 mL) phenylmethylenebis(tricyclohexylphosphine) ruthenium dichloride (285 mg, 0.35 mmol) at room temperature. The reaction mixture was degassed and purged with nitrogen, stirred at 45° C. overnight. The reaction mixture was concentrated and the residue was purified by column chromatography (mobile phase: PE/EA 10/0 to 10/1) to give compound 1-i (970 mg, 88%). LC-MS (ESI): m/z 219.1 (M−Boc+H)⁺.

### Synthesis of Compounds 1-h

1-i (970 mg, 3.05 mmol) and THF (10 mL) were added to a reaction vial at room temperature. After degassed and purged with nitrogen, and cooled in an ice-water bath, the above mixture was added a solution of borane (1M, 10 mL, 10.00 mmol) in tetrahydrofuran dropwise, then stirred at room temperature overnight after the dropwise addition. Hydrogen peroxide (30 wt %, 10 mL) was added dropwise, NaOH (61 mg, 1.53 mmol) was added and the resulting mixture was stirred for 1 hour. The reaction mixture was concentrated at room temperature at reduced pressure to remove tetrahydrofuran, extracted by adding DCM, and the organic phase was dried over anhydrous sodium sulfate, concentrated, and the residue was purified by column chromatography (mobile phase: DCM/MeOH 10/0 to 10/1) to give compound 1-h (910 mg, 89%). LC-MS (ESI): m/z 236.9 (M−Boc+H)⁺.

### Synthesis of Compound 1-g

At room temperature, to a reaction vial were added 1-h (900 mg, 2.68 mmol), DCM (20 mL), and Days-Martin reagent (2837 mg, 6.69 mmol). After degassed and purged with nitrogen, the reaction mixture was stirred at room temperature overnight. The reaction mixture was added 100 mL of dichloromethane, washed with saturated aqueous sodium bicarbonate first, then saturated aqueous sodium bisulfite, washed with water, dried over anhydrous sodium sulfate, concentrated, and the residue was purified by column chromatography (mobile phase: PE/EA 10/0 to 4/6) to give compound 1-g (700 mg, 78%). LC-MS (ESI): m/z 235.1 (M−Boc+H)⁺.

### Synthesis of Compound 1-f

A solution of methylmagnesium bromide (1M, 8.2 mL, 8.20 mmol) in Tetrahydrofuran and THF (10 mL) were added to a reaction flask at room temperature. After degassed and purged with nitrogen and cooled in an ice-water bath, the above mixture was added a solution of 1-g (2100 mg, 6.28 mmol) in THF (10 mL) dropwise. Stirred for 2 hours at room temperature after dropwise addition, the reaction mixture was quenched by pouring into saturated aqueous ammonium chloride solution, extracted with DCM. The organic phase was washed with water, dried over anhydrous sodium sulfate, concentrated and the residue was purified by column chromatography (mobile phase: PE/EA 10/0 to 5/5) to give compound 1-f (1500 mg, 68%). LC-MS (ESI): m/z 251.2 (M−Boc+H)⁺.

### Synthesis of Compounds 1-e-1, 1-e-2

1-f (700 mg, 2.00 mmol) and acetonitrile (20 mL) were added to a reaction flask at room temperature. After degassed and purged with nitrogen and cooled in an ice-water bath, the above mixture was added HCl/1,4-dioxane (1M, 4 mL, 4.00 mmol). The reaction mixture was stirred for 5 hours at room temperature. The reaction mixture was concentrated, made basic by adding a solution of ammonia in methanol (7 M) and concentrated. Water was added and the aqueous phase was extracted with a mixed solution of DCM:MeOH=10:1. The organic phase was dried over anhydrous sodium sulfate, concentrated, and the residue was purified by prep-HPLC (mobile phase: water (0.01 M NH₄HCO₃)/acetonitrile 10/0 to 7/3) to give compounds 1-e-1 (250 mg, 50%) and 1-e-2 (180 mg, 36%).

Compound 1-e-1 (250 mg, 50%). LC-MS (ESI): m/z 251.2 (M+H)⁺; ¹HNMR (400 MHz, CDCl₃): δ 7.43-7.30 (5H, m), 5.22 (2H, s), 3.82 (1H, d, J=12.0 Hz), 3.28-3.14 (2H, m), 2.95-2.86 (1H, m), 1.76-1.64 (2H, m), 1.26 (3H, s).

Compound 1-e-2 (180 mg, 36%). LC-MS (ESI): m/z 251.2 (M+H)⁺; ¹HNMR (400 MHz, CDCl₃): δ 7.45-7.31 (5H, m), 5.20 (2H, s), 4.05 (1H, d, J=13.2 Hz), 3.45-3.25 (1H, m), 2.87-2.70 (2H, m), 1.70-1.60 (2H, m), 1.23 (3H, s).

### Synthesis of Compounds 1-d

To a reaction vial were added 2,4,7-trichloro-8-fluoropyrido[4,3-d]pyrimidine (303 mg, 1.20 mmol), DCM (15 mL) at room temperature. After degassed and purged with nitrogen, the above mixture was cooled to −78° C., was added DIPEA (258 mg, 2.00 mmol) and a solution of 1-e-1 (250 mg, 1.00 mmol) in THF (5 mL) dropwise. After addition, the reaction mixture was naturally warmed to room temperature overnight. The reaction mixture was added with water, and the aqueous phase was extracted with DCM. The organic phases were combined, dried over anhydrous sodium sulfate, concentrated, and the residue was purified by column chromatography (mobile phase: PE/EA 10/0 to 6/4) to give compound 1-d (300 mg, 64%). LC-MS (ESI): m/z 466.5 (M+H)⁺.

### Synthesis of Compounds 1-c

To a reaction vial were added 1-d (300 mg, 0.64 mmol), ((2R,7aS)-2-fluorohexahydro-1H-pyrrolizin-7a-yl)methanol (123 mg, 0.77 mmol), 1,4-dioxane (7 mL), DIPEA (208 mg, 1.61 mmol) at room temperature. After degassed and purged with nitrogen, the reaction mixture was stirred at 90° C. overnight. The reaction mixture was concentrated and the residue was purified by column chromatography (mobile phase: DCM/MeOH 10/0 to 10/1) to give compound 1-c (290 mg, 77%).

LC-MS (ESI): m/z 589.6 (M+H)⁺.

### Synthesis of Compound 1-b

At room temperature, to a microwave tube were added 1-c (200 mg, 0.34 mmol), 2-(8-ethyl-7-fluoro-3-(methoxymethoxy)naphthalen-1-yl)-4,4,5,5-tetramethyl-1,3,2-dioxaborolane (148 mg, 0.41 mmol), cataCXium A Pd G3 (22 mg, 0.03 mmol), potassium phosphate (217 mg, 1.02 mmol), 1,4-dioxane (2 mL), and water (0.5 mL). The mixture was degassed and purged with nitrogen, sealed, and stirred at 80° C. overnight. The reaction mixture was concentrated and the residue was purified by column chromatography (mobile phase: DCM/MeOH 10/0 to 10/1) to give compound 1-b (140 mg, 52%). LC-MS (ESI): m/z 787.2 (M+H)⁺.

### Synthesis of Compound 1-a

To a reaction vial were added 1-b (140 mg, 0.18 mmol) and acetonitrile (5 mL) at room temperature. After degassed and purged with nitrogen, the above mixture was cooled down to 0° C., was added HCl/1,4-dioxane (4.0 M, 1 mL). It was stirred at 0° C. for 3 hours. The reaction mixture was concentrated at low temperature to give the crude compound 1-a (132 mg). LC-MS (ESI): m/z 743.7 (M+H)⁺.

### Synthesis of Compound 1

At room temperature, to a reaction vial were added crude compound 1-a (132 mg, 0.18 mmol), methanol (13 mL), Pd/C 10% (130 mg). After degassed and purged with H2, and stirred at room temperature overnight. The reaction mixture was filtered, and the filtrate was concentrated and the residue was purified by column chromatography (mobile phase: DCM/MeOH(NH₃) 10/0 to 10/1) to give compound 1 (49 mg, 45%). LC-MS (ESI): m/z 609.7 (M+H)⁺.

### Synthesis of Compounds 1-1 and 1-2

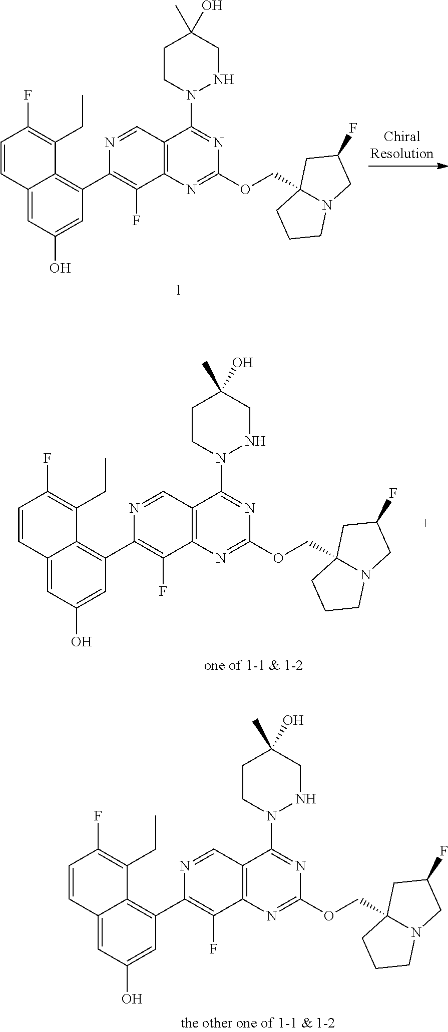 [SMILES: CCc1c(F)ccc2cc(O)cc(-c3ncc4c(N5CCC(C)(O)CN5)nc(OC[C@@]56CCCN5C[C@H](F)C6)nc4c3F)c12 ; CCc1c(F)ccc2cc(O)cc(-c3ncc4c(N5CC[C@](C)(O)CN5)nc(OC[C@@]56CCCN5C[C@H](F)C6)nc4c3F)c12 ; CCc1c(F)ccc2cc(O)cc(-c3ncc4c(N5CC[C@](C)(O)CN5)nc(OC[C@@]56CCCN5C[C@H](F)C6)nc4c3F)c12]

Compound 1 (39 mg, 0.064 mmol) was separated by chiral resolution to obtain compound 1-1(16 mg, 41%), LC-MS (ESI): m/z 609.6 (M+H)⁺; compound 1-2 (15 mg, 38%), LC-MS (ESI): m/z 609.6 (M+H)⁺.

| Conditions for chiral analysis | Conditions for chiral preparation |
|---|---|
| Instrument: UPCC(Waters) | Instrument: SFC-150 (Waters) |
| Column: IC 4.6*250 mm, 5 um (Daicel) | Column: IC 25*250 mm, 10 um (Daicel) |
| Column temperature: 40° C. | Column temperature: 35° C. |
| Mobile phase: CO₂/MeOH[0.2% NH₃(7M | Mobile phase: CO₂/MeOH[0.2% NH₃(7M in |
| in MeOH)] = 45/55 | MeOH)] = 50/50 |
| Flow rate: 3.0 mL/min | Flow rate: 100 mL/min |
| Wavelength: 214 nm | Pressure: 100 bar |
| | Wavelength: 214 nm |
| | Circulation: 6.8 min |
| | Sample solution: 39 mg in 14 mL of methanol |
| 1-1: the retention time was 1.268 min; | |
| d.e. % = 100.0%; | |
| 1-2: the retention time was 2.119 min; | |
| d.e. % = 99.94%. | |

### Example 2 Synthetic Route of Compound 2

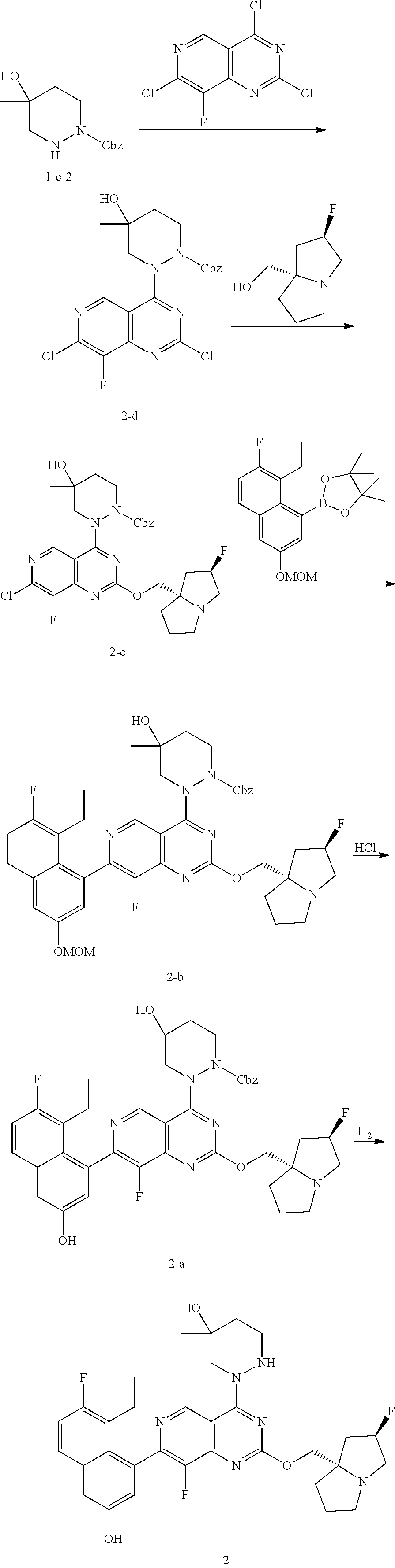

### Synthesis of Compounds 2-d

At room temperature, to a reaction vial were added 2,4,7-trichloro-8-fluoropyrido[4,3-d]pyrimidine (217 mg, 0.86 mmol) and DCM (15 mL). After degassed and purged with nitrogen, the above mixture was cooled to −78° C., was added DIPEA (186 mg, 1.44 mmol) and a solution of 1-e-2 (180 mg, 0.72 mmol) in THF (5 mL) dropwise. After addition, the reaction mixture was naturally warmed to room temperature and stirred overnight. The reaction mixture was added with water, and the aqueous phase was extracted with DCM. The organic phases were combined, dried, concentrated, and the residue was purified by column chromatography (mobile phase: PE/EA 10/0 to 6/4) to give compound 2-d (200 mg, 60%). LC-MS (ESI): m/z 466.5 (M+H)⁺.

### Synthesis of Compound 2-c

At room temperature, to a reaction vial were added 2-d (200 mg, 0.43 mmol), ((2R,7aS)-2-fluorohexahydro-1H-pyrrolizin-7a-yl)methanol (82 mg, 0.52 mmol), 1,4-dioxane (6 mL) and DIPEA (138 mg, 1.07 mmol). After degassed and purged with nitrogen, the reaction mixture was stirred at 90° C. overnight. The reaction mixture was concentrated and the residue was purified by column chromatography (mobile phase: DCM/MeOH 10/0 to 10/1) to give compound 2-c (230 mg, 91%). LC-MS (ESI): m/z 589.6 (M+H)⁺.

### Synthesis of Compound 2-b

At room temperature, to a microwave tube were added 2-c (230 mg, 0.39 mmol), 2-(8-ethyl-7-fluoro-3-(methoxymethoxy)naphthalen-1-yl)-4,4,5,5-tetramethyl-1,3,2-dioxaborolane (169 mg, 0.47 mmol), cataCXium A Pd G3 (29 mg, 0.04 mmol), potassium phosphate (248 mg, 1.17 mmol), 1,4-dioxane (2 mL) and water (0.5 mL). The mixture was degassed and purged with nitrogen, sealed, and stirred at 80° C. overnight. The reaction mixture was concentrated and the residue was purified by column chromatography (mobile phase: DCM/MeOH 10/0 to 10/1) to give compound 2-b (220 mg, 72%). LC-MS (ESI): m/z 787.7 (M+H)⁺.

### Synthesis of Compound 2-a

At room temperature, to a reaction vial were added 2-b (220 mg, 0.28 mmol), acetonitrile (5 mL). After degassed and purged with nitrogen, the above mixture was cooled down to 0° C. and was added HCl/1,4-dioxane (4.0 M, 1 mL). It was stirred at 0° C. for 3 hours. The reaction mixture was concentrated at low temperature to give the crude compound 2-a (208 mg). LC-MS (ESI): m/z 743.7 (M+H)⁺.

### Synthesis of Compound 2

At room temperature, to a reaction vial were added crude compound 2-a (208 mg, 0.28 mmol), methanol (20 mL) and Pd/C 10% (200 mg). After degassed and purged with H2, the reaction mixture was stirred at room temperature overnight. The reaction mixture was filtered, and the filtrate was concentrated, and the residue was purified by column chromatography (mobile phase: DCM/MeOH(NH₃) 10/0 to 10/1) to give compound 2 (93 mg, 55%). LC-MS (ESI): m/z 609.7 (M+H)⁺.

### Synthesis of Compounds 2-1&2-2

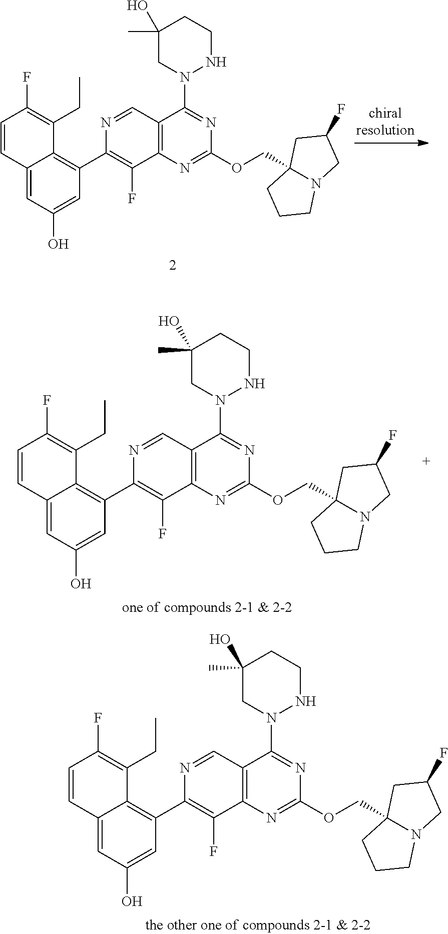 [SMILES: CCc1c(F)ccc2cc(O)cc(-c3ncc4c(N5CC(C)(O)CCN5)nc(OC[C@@]56CCCN5C[C@H](F)C6)nc4c3F)c12 ; CCc1c(F)ccc2cc(O)cc(-c3ncc4c(N5C[C@](C)(O)CCN5)nc(OC[C@@]56CCCN5C[C@H](F)C6)nc4c3F)c12 ; CCc1c(F)ccc2cc(O)cc(-c3ncc4c(N5C[C@@](C)(O)CCN5)nc(OC[C@@]56CCCN5C[C@H](F)C6)nc4c3F)c12]

Compound 2 (79 mg, 0.13 mmol) was separated by chiral resolution to obtain compound 2-1(27 mg, 34%), LC-MS (ESI): m/z 609.6 (M+H)⁺; ¹H NMR (400 MHz, DMSO-d₆): δ10.39-10.29 (1H, d, J=7.2 Hz), 9.92 (1H, s), 7.81-7.71 (1H, dd, J=8.8, 6.0 Hz), 7.40-7.28 (2H, m), 7.06-6.94 (1H, m), 5.55-5.40 (1H, m), 5.41-5.18 (1H, d, J=54.4 Hz), 4.91-4.74 (1H, m), 4.27-3.91 (2H, m), 3.30-2.94 (5H, m), 2.92-2.57 (2H, m), 2.44-1.95 (6H, m), 1.94-1.57 (5H, m), 1.27-1.14 (3H, m), 0.80-0.64 (3H, t, J=7.2 Hz); compound 2-2 (28 mg, 35%), LC-MS (ESI): m/z 609.6 (M+H)⁺.

| Conditions for chiral analysis | Conditions for chiral preparation |
|---|---|
| Instrument: UPCC(Waters) | Instrument: SFC-150 (Waters) |
| Column: IC 4.6*250 mm, 5 um (Daicel) | Column: IC 25*250 mm, 10 um (Daicel) |
| Column temperature: 40° C. | Column temperature: 35° C. |
| Mobile phase: CO₂/MeOH[0.2% NH₃(7M in | Mobile phase: CO₂/MeOH[0.2% NH₃(7M in |
| MeOH)] = 35/65 | MeOH)] = 60/40 |
| Flow rate: 3.0 mL/min | Flow rate: 100 mL/min |
| Wavelength: 214 nm | Pressure: 100 bar |
| | Wavelength: 214 nm |
| | Circulation: 7.5 min |
| | Sample solution: 79 mg was dissolved in 15 mL of |
| | methanol |
| 2-1: the retention time was 1.989 min; | |
| d.e. % = 100.0%; | |
| 2-2: the retention time was 2.766 min; | |
| d.e. % = 100.0%. | |

### Example 3 Synthetic Route of Compound 3

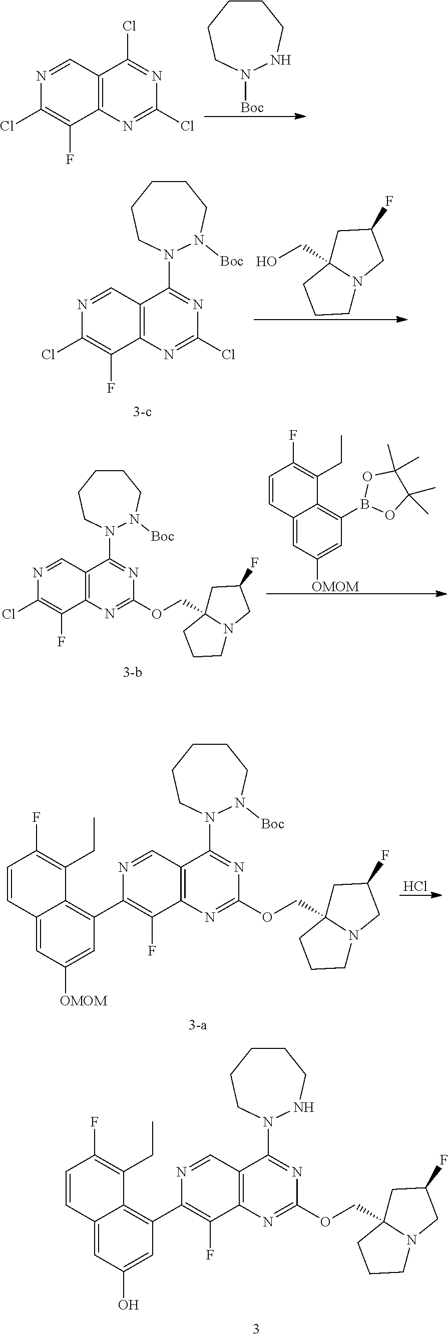

### Synthesis of Compounds 3-c

To a solution of 2,4,7-trichloro-8-fluoropyrido[4,3-d]pyrimidine (300 mg, 1.19 mmol) in dichloromethane (30 mL) was added tert-butyl 1,2-diazepine-1-carboxylate (238 mg, 1.19 mmol) and N,N-diisopropylethylamine (461 mg, 3.56 mmol) in a a dry ice-acetone bath, and the mixture was stirred for 1 hour at this temperature. The reaction mixture was warmed to room temperature and continued stirring for 1 h. Water was added and the aqueous phase was extracted with dichloromethane (50 mL*2), the combined organic phases were dried over anhydrous sodium sulfate, filtered and concentrated to dryness to give compound 3-c (490 mg, 99%). LC-MS (ESI): m/z 416.0 (M+H)⁺.

### Synthesis of Compound 3-b

To a solution of 3-c (490 mg, 1.18 mmol) in dichloromethane (20 mL) was added ((2R,7aS)-2-fluorohexahydro-1H-pyrrolizin-7a-yl)methanol (281 mg, 1.77 mmol) in an ice-water bath and followed by slow addition of sodium tert-butoxide (226 mg, 2.35 mmol) and the mixture was stirred for 2 h at this temperature. Then water was added to the mixture and the aqueous phase was extracted with dichloromethane (50 mL*2), the combined organic phases were dried over anhydrous sodium sulfate, filtered, and the filtrate was concentrated to dryness and the crude was purified by flash column chromatography (mobile phase: dichloromethane: methanol=10:1/dichloromethane, 0-50%) to give compound 3-b (561 mg, 88%). LC-MS (ESI): m/z 539.4 (M+H)⁺.

### Synthesis of Compound 3-a

To a 10 mL microwave tube were added 3-b (70 mg, 0.13 mmol), 2-(8-ethyl-7-fluoro-3-(methoxymethoxy)naphthalen-1-yl)-4,4,5,5-tetramethyl-1,3,2-dioxaborolane (70 mg, 0.20 mmol), methanesulfonicacid[n-butylbis(1-adamantyl)phosphine](2-amino-1,1′-biphenyl-2-yl)palladium(II) (9 mg, 0.013 mmol), potassium phosphate (83 mg, 0.39 mmol), tetrahydrofuran (5 mL) and water (1 mL). After degassed and purged with nitrogen several times, the microwave tube was sealed. The reaction mixture was stirred at 65° C. overnight. Water was added to the reaction mixture and the aqueous phase was extracted with ethyl acetate (50 mL). The organic phase was dried over anhydrous sodium sulfate, filtered, and the filtrate was concentrated to dryness and the crude was purified by flash column chromatography (mobile phase: methanol/dichloromethane, 0-100%) to give compound 3-a (74 mg, 77%). LC-MS (ESI): m/z 737.8 (M+H)⁺.

### Synthesis of Compound 3

To a solution of 3-a (74 mg, 0.1 mmol) in acetonitrile (15 mL) was added HCl/1,4-dioxane (4M, 3 mL)in an ice-water bath. The temperature was slowly warmed to room temperature and the mixture was stirred at this temperature overnight. Saturated aqueous sodium bicarbonate solution was added and the aqueous phase was extracted with ethyl acetate (50 mL). The organic phase was dried over anhydrous sodium sulfate, filtered, the filtrate was concentrated to dryness and the crude was purified by prep-HPLC to give compound 3 (13.5 mg, 23%). LC-MS (ESI): m/z 593.3 (M+H)⁺.

### Example 4 Synthetic Route of Compound 4

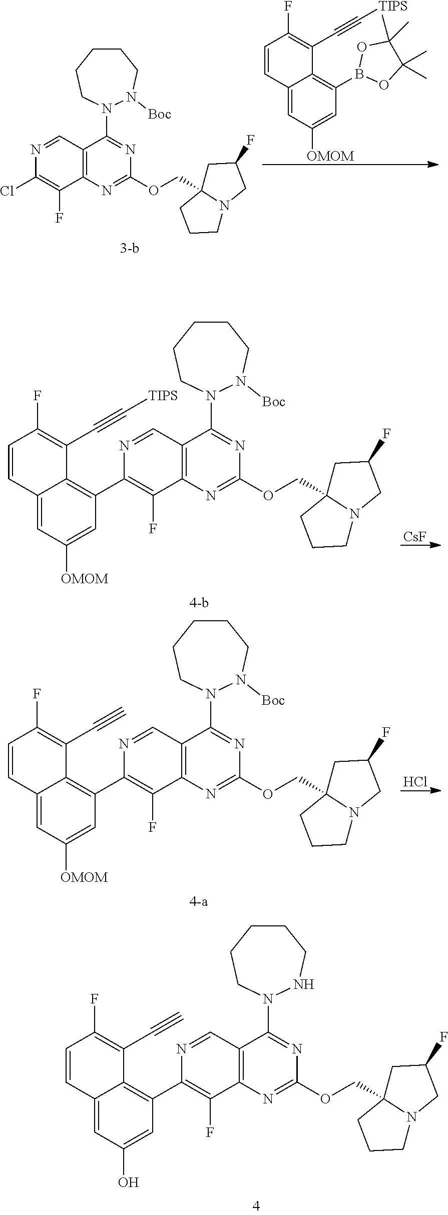

### Synthesis of Compound 4-b

To a microwave tube (30 mL) were added 3-b (150 mg, 0.28 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (171 mg, 0.33 mmol), methanesulfonato[n-butylbis(1-adamantyl) phosphine](2-amino-1,1′-biphenyl-2-yl)palladium(II) (20 mg, 0.028 mmol), potassium phosphate (177 mg, 0.83 mmol), tetrahydrofuran (15 mL) and water (3 mL). The microwave tube was sealed, degassed and purged with nitrogen several times and stirred at 65° C. overnight. The reaction mixture was added water, extracted with ethyl acetate (50 mL*2), the combined organic phases were dried over anhydrous sodium sulfate, filtered, and the filtrate was concentrated to dryness and the crude was purified by flash column chromatography (mobile phase: methanol/dichloromethane, 0-20%) to obtain 4-b (244 mg, 99%). LC-MS (ESI): m/z 889.9 (M+H)⁺.

### Synthesis of Compound 4-a

Cesium fluoride (417 mg, 2.74 mmol) was added to a solution of 4-b (244 mg, 0.27 mmol) in N,N-dimethylformamide (10 mL) at room temperature and the mixture was stirred for 1 h at room temperature. Water was added and the aqueous phase was extracted with ethyl acetate (50 mL*2), the combined organic phases were washed twice with water and twice with brine sequentially, dried over anhydrous sodium sulfate, filtered, and the filtrate was concentrated to dryness to give compound 4-a (200 mg, 99%). LC-MS (ESI): m/z 733.8 (M+H)*.

### Synthesis of Compound 4

To a solution of 4-a (200 mg, 0.27 mmol) in acetonitrile (15 mL) was added HCl/1,4-dioxane (4M, 5 mL)in an ice-water bath. The mixture was slowly warmed to room temperature and stirred at this temperature overnight. The reaction mixture was added saturated aqueous sodium bicarbonate solution and the aqueous phase was extracted with ethyl acetate (30 mL*2), the combined organic phases were dried over anhydrous sodium sulfate, filtered, concentrated to dryness, and the crude was purified by prep-HPLC to give compound 4 (120 mg, 75%). LC-MS (ESI): m/z 589.3 (M+H)⁺;

¹H NMR (400 MHz, CDCl₃): δ 10.15 (1H, d, J=34.8 Hz), 7.55-7.46 (1H, m), 7.25 (1H, dd, J=38.4, 1.6 Hz), 7.15-7.07 (1H, m), 7.02 (1H, s), 5.31 (1H, d, J=54.0 Hz), 4.43-4.31 (1H, m), 4.30-4.16 (2H, m), 4.06-3.92 (2H, m), 3.41-3.07 (4H, m), 3.06-2.96 (2H, m), 2.79 (1H, d, J=4.4 Hz), 2.46-2.10 (4H, m), 2.03-1.85 (4H, m), 1.78-1.57 (4H, m).

### Example 5 Synthetic Route of Compound 5

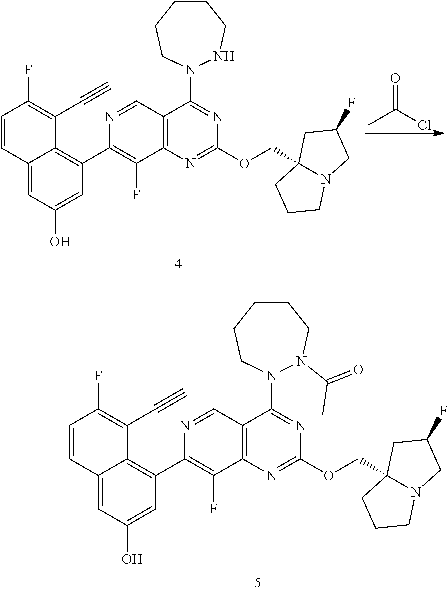

### Synthesis of Compound 5

To a solution of compound 4 (20 mg, 0.034 mmol) in dichloromethane (8 mL) was slowly added acetyl chloride (13 mg, 0.17 mmol) and N,N-diisopropylethylamine (44 mg, 0.34 mmol) in an ice-water bath, and the mixture was stirred for 2 hr at 0° C. The mixture was concentrated to dryness, dissolved in methanol (6 mL), was added potassium carbonate (94 mg, 0.68 mmol) and the mixture was stirred for 2 hours at room temperature. To the reaction mixture, water was added and the aqueous phase was extracted with ethyl acetate (30 mL*2), the combined organic phases were dried over anhydrous sodium sulfate, filtered, the filtrate was concentrated to dryness and the crude was purified by prep-HPLC to give compound 5 (8.8 mg, 41%). LC-MS (ESI): m/z 631.3 (M+H)⁺.

### Example 6 Synthetic Route of Compound 6

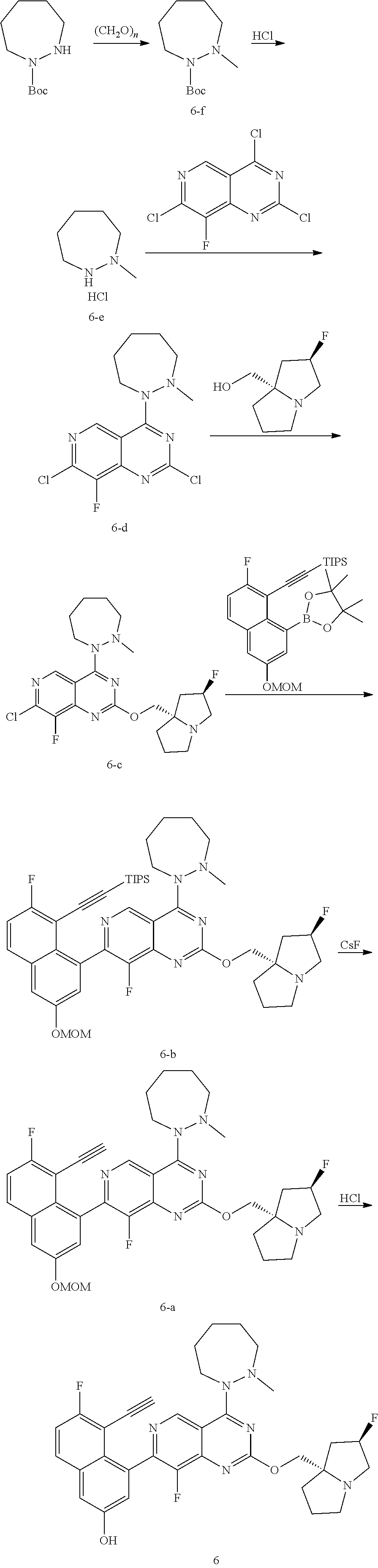

### Synthesis of Compound 6-f

A reaction vial charged with tert-butyl 1,2-diazepane-1-carboxylate (200 mg, 0.999 mmol), methanol (20 mL), paraformaldehyde (270 mg, 2.997 mmol), and sodium cyanoborohydride (188 mg, 2.996 mmol) was stirred at room temperature overnight under atmosphere of nitrogen. The next day, the reaction mixture was concentrated to dryness and the residue was purified by column chromatography (mobile phase: ethyl acetate/petroleum ether 0/100 to 20/80) to give compound 6-f (189 mg, 88%). LC-MS (ESI): m/z=215.2 (M+H)⁺.

### Synthesis of Compound 6-e

To a reaction vial were added 6-f (188 mg, 0.877 mmol), acetonitrile (20 mL) and hydrochloric acid/1,4-dioxane (4 M, 2 mL), and the mixture was stirred at room temperature overnight under atmosphere of nitrogen. The next day, the reaction mixture was directly concentrated to dryness to give compound 6-e (132 mg, 99.9%), which was used directly for the next step without purification.

LC-MS (ESI): m/z=115.2 (M+H)⁺.

### Synthesis of Compound 6-d

To a reaction vial were added 6-e (140 mg, 0.929 mmol), dichloromethane (30 mL), diisopropylethylamine (0.461 mL, 2.788 mmol). Cooled in a dry ice-acetone bath, the above mixture was added 2,4,7-trichloro-8-fluoropyrido[4,3-d]pyrimidine (235 mg, 0.929 mmol) and stirred for 1 hr under atmosphere of nitrogen. Quenched with water and the aqueous phase was extracted with dichloromethane (40 mL*2). The organic phase was dried, concentrated to dryness, and the residue was purified by column chromatography(mobile phase: ethyl acetate/petroleum ether 0/100 to 30/70) to give compound 6-d (127 mg, 41%). LC-MS (ESI): m/z=330.7 (M+H)⁺.

### Synthesis of Compound 6-c

To a reaction vial charged with 6-d (120 mg, 0.363 mmol), dichloromethane (20 mL), ((2R,7aS)-2-fluorohexahydro-1H-pyrrolozin-7a-yl)methanol (87 mg, 0.544 mmol) was added sodium tert-butoxide (87 mg, 0.909 mmol) in an ice-water bath, and the reaction mixture was stirred for 3 hours, then was added ((2R,7aS)-2-fluorohexahydro-1H-pyrrolizin-7a-yl)methanol (87 mg, 0.544 mmol) and sodium tert-butanolate (87 mg, 0.909 mmol) and continued stirring for 2 hours. The reaction mixture was quenched with water and the aqueous phase was extracted with dichloromethane (40 mL*2). The organic phase was dried over anhydrous sodium sulfate, concentrated to dryness, and the residue was purified by column chromatography (mobile phase: ammonia methanol/dichloromethane 0/100 to 6/94) to give compound 6-c (80 mg, 49%). LC-MS (ESI): m/z=453.6(M+H)⁺.

### Synthesis of Compound 6-b

A microwave tube charged with 6-c (120 mg, 0.265 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (204 mg, 0.397 mmol), tetrahydrofuran (12 mL), water (1.2 mL), potassium phosphate (169 mg, 0.795 mmol) and cataCXium A Pd G3 (19 mg, 0.026 mmol) was degassed and purged with nitrogen for three times, then sealed and the reaction mixture was stirred at 65° C. overnight. The next day, the reaction mixture was concentrated to dryness and the residue was purified by column chromatography (mobile phase: methanol/dichloromethane 0/100 to 6/94) to give compound 6-b (199 mg, 94%).

LC-MS (ESI): m/z=803.8 (M+H)⁺.

### Synthesis of Compound 6-a

A reaction flask charged with 6-b (199 mg, 0.248 mmol), DMF (10 mL) and cesium fluoride (376 mg, 2.478 mmol) with stirred for 1 h at room temperature under atmosphere of nitrogen. The reaction mixture was quenched with water and the aqueous phase was extracted with ethyl acetate (40 mL*2). The organic phase was washed with brine (30 mL*3), dried over anhydrous sodium sulfate, and concentrated to dryness to give compound 6-a (169 mg), which was used directly in the next step without purification. LC-MS (ESI): m/z=647.7 (M+H)⁺.

### Synthesis of Compound 6

A reaction vial charged with 6-a (169 mg, 0.261 mmol), acetonitrile (20 mL), 1,4-dioxane/hydrochloride (4M, 2 mL, 8 mmol) was stirred at room temperature for 2 hours. The reaction mixture was concentrated to dryness, and the residue was added methanol (10 mL) and adjusted to pH 7 with saturated sodium bicarbonate solution. The mixture was concentrated to dryness and the residue was purified by column chromatography (mobile phase: methanol/dichloromethane 0/100 to 6/94) to give compound 6 (90 mg, 57%). LC-MS (ESI): m/z=603.7 (M+H)⁺;¹H NMR (400M, CD₃OD) δ: 10.18 (1H, d, J=10.0 Hz), 7.90-7.80 (1H, m), 7.38-7.27 (2H, m), 7.22 (1H, s), 5.34 (1H, d, J=54.0 Hz), 4.59 (1H, d, J=14.4 Hz), 4.39-4.21 (2H, m), 3.68-3.57 (1H, m), 3.54-3.31 (3H, m), 3.13-3.01 (1H, m), 2.86- 2.76 (1H, m), 2.73 (3H, s), 2.45-2.10 (3H, m), 2.09-1.75 (7H, m), 1.64-1.22 (4H, m).

### Example 7 Synthetic Route of Compound 7

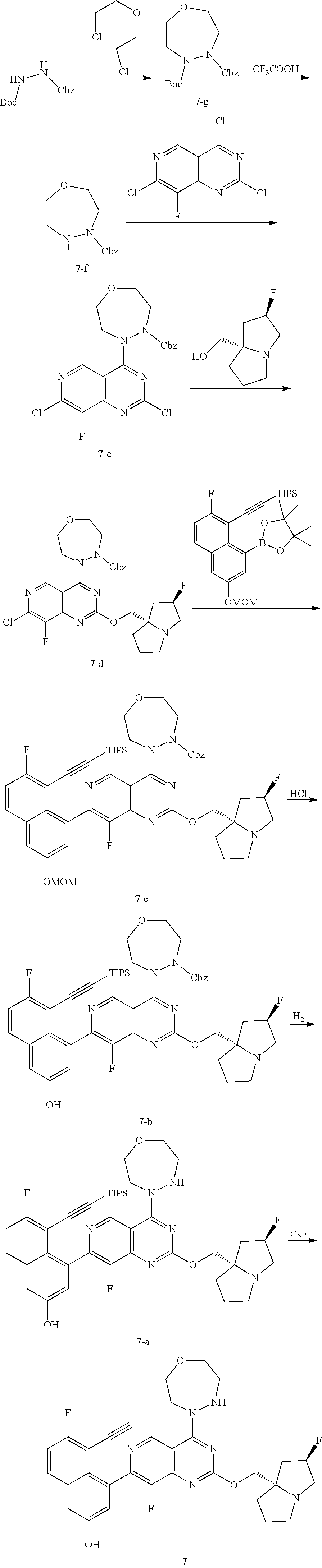

### Synthesis of Compound 7-g

NaH (60%, 751 mg, 18.78 mmol) was added into a reaction flask charged with DMF (20 mL) under atmosphere of nitrogen in an ice-water bath. After stirred for 15 min, the above mixture was added a solution of 2-(tert-butyl)hydrazine-1,2-dicarboxylic acid-1-benzyl ester (2000 mg, 7.51 mmol) in DMF (5 mL) dropwise. After addition, the above mixture was warmed to room temperature and stirred for 1 hour, then cooled in an ice-water bath, and was added a solution of 2,2′-dichlorodiethyl ether (1149 mg, 8.04 mmol) in DMF (5 mL) dropwise. The reaction mixture was warmed to room temperature overnight. The reaction mixture was quenched with saturated aqueous ammonium chloride solution and the aqueous phase was extracted with ethyl acetate. The organic phases were combined, washed with water, dried over anhydrous sodium sulfate, concentrated, and the residue was purified by column chromatography (mobile phase: PE/EA 10/0 to 10/3) to give compound 7-g (1300 mg, 51%). LC-MS (ESI): m/z 237.1 (M−Boc+H)⁺.

### Synthesis of Compound 7-f

A reaction vial charged with 7-g (1300 mg, 3.86 mmol), DCM (10 mL) and trifluoroacetic acid (4467 mL, 39.18 mmol) was stirred at room temperature for 3 hours. The reaction mixture was concentrated and adjusted to basic by addition of ammonia in methanol (7 M), concentrated and the residue was purified by column chromatography (mobile phase: DCM/MeOH 10/0 to 10/1) to give compound 7-f (400 mg, 44%). LC-MS (ESI): m/z 237.2 (M+H)⁺.

### Synthesis of Compound 7-e

At room temperature, to a reaction vial were added 2,4,7-trichloro-8-fluoropyrido[4,3-d]pyrimidine (256 mg, 1.01 mmol), DCM (15 mL). After degassed and purged with nitrogen, the above mixture was cooled to −78° C. in a dry ice-acetone bath, then was added DIPEA (274 mg, 2.12 mmol), 7-f (200 mg, 0.85 mmol). After addition, the reaction mixture was naturally warmed to room temperature and stirred overnight. The reaction mixture was added with water, and the aqueous phase was extracted with DCM. The organic phases were combined, dried over anhydrous sodium sulfate, concentrated, and the residue was purified by column chromatography (mobile phase: PE/EA 10/0 to 6/4) to give compound 7-e (300 mg, 78%).

### Synthesis of Compound 7-d

At room temperature, to a reaction vial were added 7-e (300 mg, 0.66 mmol), ((2R,7aS)-2-fluorohexahydro-1H-pyrrolizin-7a-yl)methanol (137 mg, 0.86 mmol), 1,4-dioxane (10 mL) and DIPEA (214 mg, 1.65 mmol). After degassed and purged with nitrogen, the reaction mixture was stirred at 90° C. overnight. The reaction mixture was concentrated and the residue was purified by column chromatography (mobile phase: DCM/MeOH 10/0 to 10/1) to give compound 7-d (240 mg, 63%). LC-MS (ESI): m/z 575.5 (M+H)⁺.

### Synthesis of Compound 7-c

At room temperature, to a microwave tube were added 7-d (130 mg, 0.23 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (139 mg, 0.27 mmol), cataCXium A Pd G3 (16 mg, 0.02 mmol), potassium phosphate (144 mg, 0.68 mmol), THF (2 mL) and water (0.5 mL). The mixture was degassed and purged with nitrogen, sealed, and stirred at 65° C. overnight. The reaction mixture was concentrated and the residue was purified by column chromatography (mobile phase: DCM/MeOH 10/0 to 10/1) to give compound 7-c (150 mg, 72%). LC-MS (ESI): m/z 925.4 (M+H)⁺.

### Synthesis of Compound 7-b

At room temperature, to a reaction flask were added 7-c (130 mg, 0.14 mmol) and acetonitrile (5 mL). After degassed and purged with nitrogen, and cooled to 0° C., the above mixture was added HCl/1,4-dioxane (4.0 M, 1 mL). The reaction mixture was stirred at 0° C. for 3 hours, concentrated at low temperature to give the crude compound 7-b (150 mg). LC-MS (ESI): m/z 881.4 (M+H)⁺.

### Synthesis of Compound 7-a

At room temperature, to a reaction vial were added crude compound 7-b (150 mg, 0.14 mmol), methanol (14 mL) and Pd/C 10% (120 mg). The reaction mixture was degassed and purged with H2 and stirred at room temperature overnight. The reaction mixture was filtered and the filtrate was concentrated to give the crude compound 7-a (105 mg). LC-MS (ESI): m/z 747.4 (M+H)⁺.

### Synthesis of Compound 7

At room temperature, to a reaction flask were added crude compound 7-a (105 mg, 0.14 mmol), DMF (3 mL) and cesium fluoride (107 mg, 0.70 mmol). After degassed and purged with nitrogen, the reaction mixture was stirred at 60° C. for 5 hours. The reaction mixture was filtered and the filtrate was purified by prep-HPLC to give compound 7 (12 mg, 14%). LC-MS (ESI): m/z 591.6 (M+H)⁺.

### Example 8 Synthetic Route of Compound 8

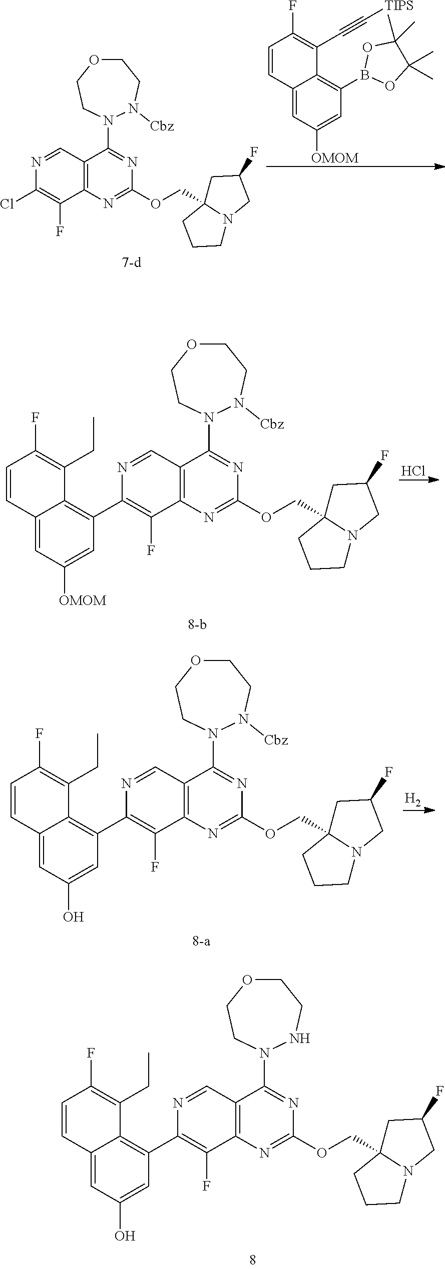

### Synthesis of Compound 8-b

At room temperature, to a microwave tube were added 7-d (100 mg, 0.17 mmol), 2-(8-ethyl-7-fluoro-3-(methoxymethoxy)naphthalen-1-yl)-4,4,5,5-tetramethyl-1,3,2-dioxaborolane (75 mg, 0.21 mmol), cataCXium A Pd G3 (13 mg, 0.02 mmol), potassium phosphate (111 mg, 0.52 mmol), THF (2 mL) and water (0.5 mL). The mixture was degassed and purged with nitrogen, sealed, and stirred at 65° C. overnight. The reaction mixture was concentrated and the residue was purified by column chromatography (mobile phase: DCM/MeOH 10/0 to 10/1) to give compound 8-b (110 mg, 82%).

LC-MS (ESI): m/z 773.8 (M+H)⁺.

### Synthesis of Compound 8-a

At room temperature, to a reaction vial were added 8-b (110 mg, 0.14 mmol) and acetonitrile (5 mL). After degassed and purged with nitrogen, the above mixture was cooled down to 0° C., the mixture was added HCl/1,4-dioxane (4.0 M, 1 mL), then stirred at 0° C. for 3 hours. The reaction mixture was concentrated at low temperature to give the crude compound 8-a (104 mg). LC-MS (ESI): m/z 729.7 (M+H)⁺.

### Synthesis of Compound 8

At room temperature, to a reaction vial were added crude 8-a (104 mg, 0.14 mmol), methanol (10 mL) and Pd/C 10% (100 mg). After degassed and purged with H2, the reaction mixture was stirred at room temperature overnight. The reaction mixture was filtered, and the filtrate was concentrated and the residue was purified by prep-HPLC to give compound 8 (18 mg, 21%). LC-MS (ESI): m/z 595.7 (M+H)⁺; ¹H NMR (400 MHz, DMSO-d₆): δ 10.31 (1H, s), 9.97 (1H, s), 7.82-7.70 (1H, dd, J=9.2, 6.4 Hz), 7.42-7.27 (2H, m), 7.06-6.99 (1H, d, J=2.0 Hz), 6.25-6.15 (1H, t, J=6.0 Hz), 5.40-5.16 (1H, d, J=53.6 Hz), 4.43-3.89 (6H, m), 3.86-3.68 (2H, t, J=4.4 Hz), 3.23-2.94 (5H, m), 2.89-2.76 (1H, m), 2.43-2.24 (1H, m), 2.23-1.64 (7H, m), 0.72 (3H, t, J=7.2 Hz).

### Example 9 Synthetic Route of Compound 9

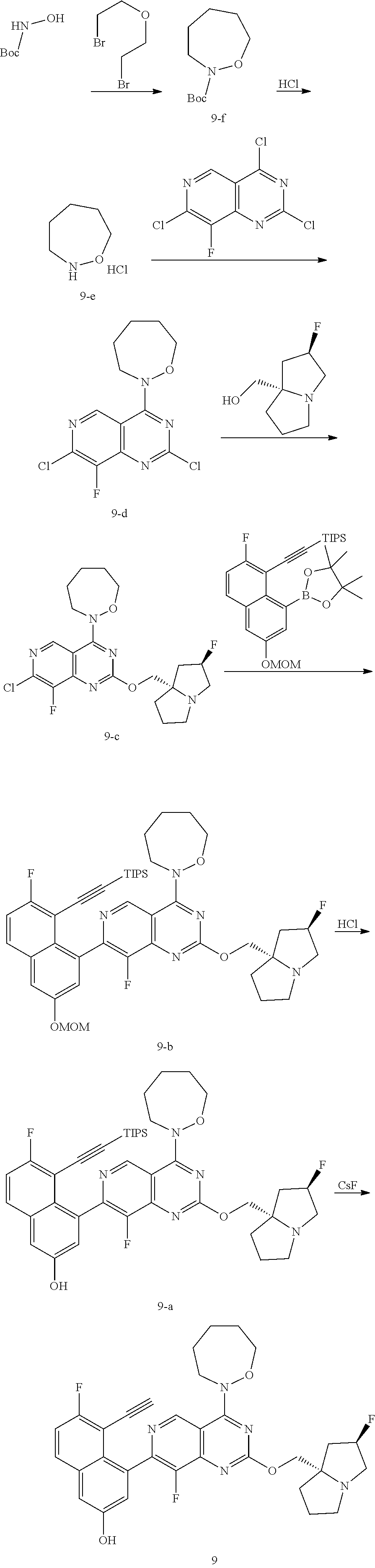

### Synthesis of Compound 9-f

Potassium tert-butoxide (2243 mg, 20.00 mmol), THF (40 mL) were added to a reaction flask at room temperature. After degassed and purged with nitrogen, the above mixture was added N-Boc hydroxylamine (1331 mg, 10.00 mmol) batchwise, and stirred for 1 hour at room temperature after addition, then was added a solution of 1,5-dibromopentane (2299 mg, 10.00 mmol) in THF (10 mL) dropwise. After dropwise addition, the reaction mixture was stirred at 75° C. overnight. The reaction mixture was added 100 mL of water and 100 mL of ethyl acetate. The mixture was partitioned and the aqueous phase was extracted with ethyl acetate. The organic phases were combined, washed with water, dried over anhydrous sodium sulfate, concentrated, and the residue was purified by column chromatography (mobile phase: PE/EA 10/0 to 10/1) to give compound 9-f (900 mg, 45%).

LC-MS (ESI): m/z 146.2 (M-t-Bu+H)⁺.

### Synthesis of Compound 9-e

Compound 9-f (200 mg, 0.99 mmol) and acetonitrile (5 mL) were added in a reaction flask at room temperature. After degassed and purged with nitrogen, the above mixture was cooled down to 0° C., the mixture was added HCl/1,4-dioxane (4.0 M, 1 mL), then stirred at 0° C. for 3 hours. The reaction mixture was concentrated at low temperature and water and ethyl acetate were added. The liquid was partitioned and the aqueous phase was lyophilized to give compound 9-e (65 mg, 48%). LC-MS (ESI): m/z 102.2 (M−HCl+H)⁺.

### Synthesis of Compound 9-d

2,4,7-Trichloro-8-fluoropyrido[4,3-d]pyrimidine (143 mg, 0.57 mmol) and DCM (10 mL) were added to a reaction vial at room temperature. After degassed and purged with nitrogen, the above mixture was cooled to −78° C. in a dry ice-acetone bath and was added DIPEA (183 mg, 1.42 mmol) and 9-e (65 mg, 0.47 mmol). After addition, the reaction mixture was naturally warmed to room temperature and stirred overnight. The reaction mixture was added with water, and the aqueous phase was extracted with DCM. The organic phases were combined, dried over anhydrous sodium sulfate, concentrated, and the residue was purified by column chromatography (mobile phase: PE/EA 10/0 to 6/4) to give compound 9-d (90 mg, 60%). LC-MS (ESI): m/z 317.5 (M+H)⁺.

### Synthesis of Compound 9-c

At room temperature, to a reaction vial were added 9-d (90 mg, 0.28 mmol), ((2R,7aS)-2-fluorohexahydro-1H-pyrrolizin-7a-yl)methanol (68 mg, 0.43 mmol), 1,4-dioxane (5 mL) and DIPEA (110 mg, 0.85 mmol). After degassed and purged with nitrogen, the reaction mixture was stirred at 90° C. overnight, then was concentrated and the residue was purified by column chromatography (mobile phase: DCM/MeOH 10/0 to 10/1) to give compound 9-c (80 mg, 64%).

### Synthesis of Compound 9-b

At room temperature, to a microwave tube were added 9-c (50 mg, 0.11 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (70 mg, 0.14 mmol), cataCXium A Pd G3 (8 mg. 0.01 mmol), potassium phosphate (72 mg, 0.34 mmol), THF (2 mL), and water (0.5 mL). The mixture was degassed and purged with nitrogen, sealed, and stirred at 65° C. overnight. The reaction mixture was concentrated and the residue was purified by column chromatography (mobile phase: DCM/MeOH 10/0 to 10/1) to give compound 9-b (55 mg, 61%). LC-MS (ESI): m/z 790.8 (M+H)⁺.

### Synthesis of Compound 9-a

At room temperature, to a reaction vial were added 9-b (55 mg, 0.07 mmol) and acetonitrile (5 mL). After degassed and purged with nitrogen, the above mixture was cooled down to 0° C., was added HCl/1,4-dioxane (4.0 M, 1 mL). It was stirred at 0° C. for 3 hours. The reaction mixture was concentrated at low temperature to give the crude compound 9-a (60 mg). LC-MS (ESI): m/z 746.8 (M+H)⁺.

### Synthesis of Compound 9

At room temperature, to a reaction flask were added crude compound 9-a (60 mg, 0.07 mmol), DMF (3 mL), cesium fluoride (106 mg, 0.70 mmol). After degassed and purged with nitrogen, the reaction mixture was stirred at 60° C. for 3 hours. The reaction mixture was filtered and the filtrate was purified by prep-HPLC to give compound 9 (8 mg, 19%). LC-MS (ESI): m/z 590.2 (M+H)⁺.

### Example 10 Synthetic Route of Compound 10

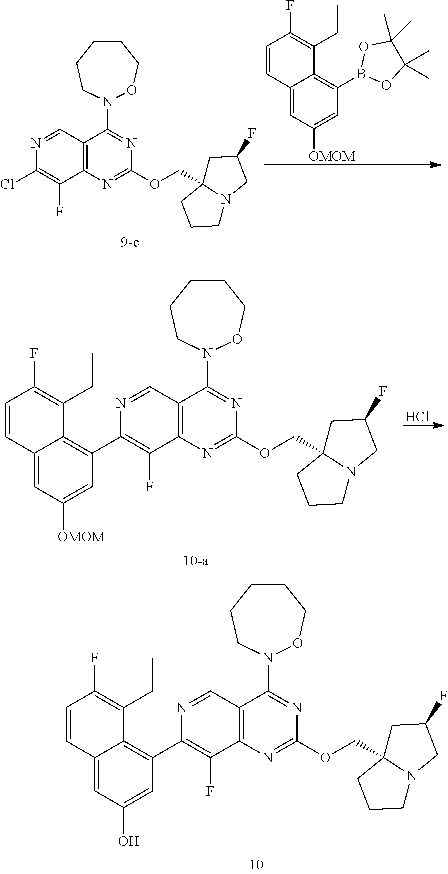

### Synthesis of Compound 10-a

At room temperature, to a microwave tube were added 9-c (15 mg, 0.03 mmol), 2-(8-ethyl-7-fluoro-3-(methoxymethoxy)naphthalen-1-yl)-4,4,5,5-tetramethyl-1,3,2-dioxaborolane (15 mg, 0.04 mmol), cataCXium A Pd G3 (3 mg, 0.01 mmol), phosphate potassium (22 mg, 0.10 mmol), THF (2 mL), and water (0.5 mL). The mixture was degassed and purged with nitrogen, sealed, and stirred at 65° C. overnight. The reaction mixture was concentrated and the residue was purified by column chromatography (mobile phase: DCM/MeOH 10/0 to 10/1) to give compound 10-a (15 mg, 69%).

LC-MS (ESI): m/z 638.8 (M+H)⁺.

### Synthesis of Compound 10

At room temperature, to a reaction flask were added 10-a (15 mg, 0.02 mmol) and acetonitrile (2 mL). After degassed and purged with nitrogen, the above mixture was cooled down to 0° C., the mixture was added HCl/1,4-dioxane (4.0 M, 0.3 mL). The reaction mixture was stirred at 0° C. for 3 hours. The reaction mixture was concentrated and the residue was purified by prep-HPLC to give compound 10 (1.3 mg, 9%). LC-MS (ESI): m/z 594.7 (M+H)⁺.

### Example 11 Synthetic Route of Compound 11

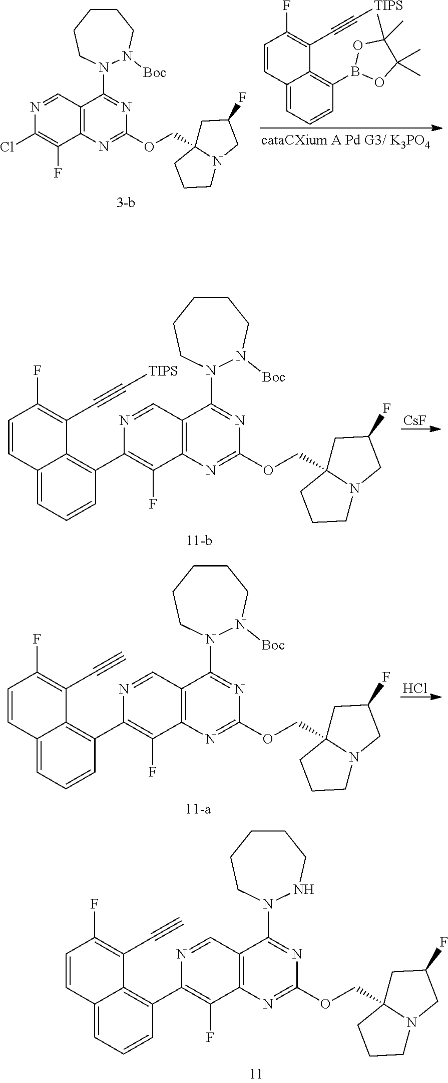

### Synthesis of Compound 11-b

To a 30 mL microwave tube were added 3-b (50 mg, 0.093 mmol), ((2-fluoro-8-(4,4,5,5-tetramethyl-1,3,2-dioxaborolan-2-yl)naphthalen-1-yl)ethynyl)triisopropylsilane (46 mg, 0.10 mmol), methanesulfonic acid [butylbis(1-adamantyl)phosphine] (2-amino-1,1′-biphenyl-2-yl)palladium(II) (7 mg, 0.009 mmol), potassium phosphate (59 mg, 0.28 mmol), tetrahydrofuran (6 mL) and water (1.5 mL). After degassed and purged with nitrogen several times, the microwave tube was sealed. The reaction mixture was stirred at 65° C. and stirred overnight. To the reaction mixture, water was added and the aqueous phase was extracted with ethyl acetate (50 mL). The organic phase was dried over anhydrous sodium sulfate, filtered, and the filtrate was concentrated to dryness and the crude was purified by flash column chromatography (mobile phase: dichloromethane: methanol=10:1/dichloromethane, 0-30%) to give compound 11-b (61 mg, 79%).

### Synthesis of Compound 11-a

To a solution of 11-b (61 mg, 0.074 mmol) in DMF (8 mL) was added cesium fluoride (112 mg, 0.74 mmol) at room temperature. The reaction mixture was stirred at room temperature overnight. Water was added and the aqueous phase was extracted with ethyl acetate (50 mL). The organic phase was washed twice with brine, dried over anhydrous sodium sulfate, filtered, and the filtrate was concentrated to dryness to give compound 11-a (49 mg, 99%). LC-MS (ESI): m/z 673.8 (M+H)⁺.

### Synthesis of Compound 11

To a solution of 11-a (49 mg, 0.073 mmol) in acetonitrile (8 mL) was added a HCl/1,4-dioxane (4 M, 1 mL) at room temperature. The reaction mixture was stirred at room temperature overnight. The reaction mixture was concentrated to dryness and the crude was purified by prep-HPLC to give compound 11 (7.5 mg, 18%). LC-MS (ESI): m/z 573.3 (M+H)⁺.

### Example 12 Synthetic Route of Compound 12

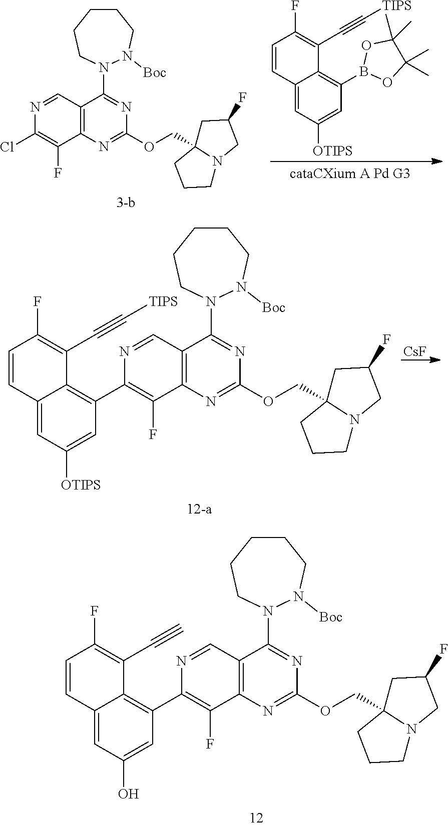

### Synthesis of Compound 12-a

To a 10 mL microwave tube were added 3-b (50 mg, 0.093 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (64 mg, 0.10 mmol), methanesulfonated [butylbis(1-adamantyl) alkyl)phosphine](2-amino-1,1′-biphenyl-2-yl)palladium(II) (7 mg, 0.009 mmol), potassium phosphate (59 mg, 0.28 mmol), tetrahydrofuran (2.5 mL) and water (0.5 mL). After degassed and purged with nitrogen several times, the microwave tube was sealed. The reaction mixture was stirred at 65° C. overnight. To the reaction mixture, water was added, the aqueous phase was extracted with ethyl acetate (50 mL). The organic phase was dried over anhydrous sodium sulfate, filtered, and the filtrate was concentrated to dryness and the crude product was purified by flash column chromatography (mobile phase: methanol/dichloromethane, 0-100%) to obtain 12-a (89 mg, 96%).

### Synthesis of Compound 12

To a solution of 12-a (89 mg, 0.089 mmol) in DMF (8 mL) was added cesium fluoride (68 mg, 0.45 mmol) at room temperature, and the reaction mixture was stirred at room temperature overnight. Water was added to the reaction mixture and the aqueous phase was extracted with ethyl acetate (30 mL). The organic phase was washed twice with brine, dried over anhydrous sodium sulfate, filtered, and the organic phase was concentrated to dryness, and the crude was purified by prep-HPLC to give compound 12 (35 mg, 57%). LC-MS (ESI): m/z 689.3 (M+H)⁺; ¹H NMR (400 MHz, CDCl₃): δ 9.20-9.10 (1H, m), 7.64-7.55 (1H, m), 7.27-7.10 (3H, m), 5.30 (1H, d, J=54.0 Hz), 4.37-4.14 (4H, m), 3.99-3.83 (1H, m), 3.35-3.10 (4H, m), 3.04-2.95 (1H, m), 2.87-2.74 (1H, m), 2.36-1.58 (13H, m), 1.61-1.41 (9H, m).

### Example 13 Synthetic Route of Compound 13 and 14

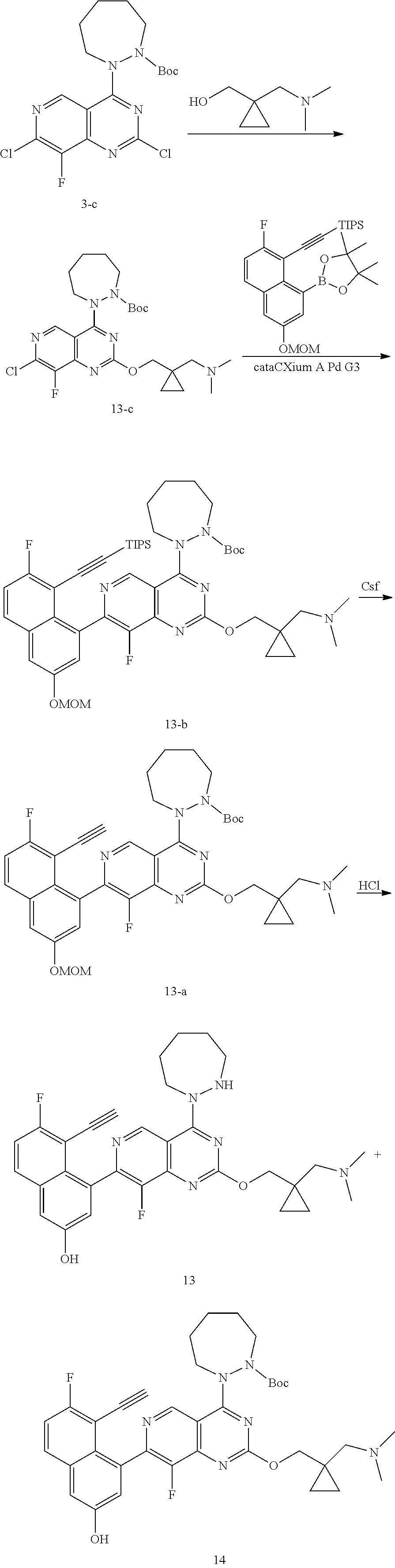

### Synthesis of Compound 13-c

To a solution of 3-c (150 mg, 0.36 mmol) in dichloromethane (15 mL) was added {1-[(dimethylamino)methyl]cyclopropyl}methanol (70 mg, 0.54 mmol), followed by slow addition of sodium tert-butoxide (69 mg, 0.72 mmol) in an ice-water bath. The temperature was slowly warmed to room temperature and the reaction mixture was stirred at room temperature overnight. Water was added to the reaction mixture and the aqueous phase was extracted with dichloromethane (30 mL). The organic phase was dried over anhydrous sodium sulfate, filtered, and the organic phase was concentrated to dryness. The crude was purified by flash column chromatography (mobile phase: DCM: MeOH=10:1/DCM, 0-50%) to give compound 13-c (140 mg, 76%). LC-MS (ESI): m/z 509.5 (M+H)⁺.

### Synthesis of Compound 13-b

To a 10 mL microwave tube were added 13-c (140 mg, 0.28 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (169 mg, 0.33 mmol), methanesulfonic acid [n-butylbis(1-adamantyl )phosphine](2-amino-1,1′-biphenyl-2-yl)palladium(II)cataCXium A Pd G3 (20 mg, 0.028 mmol), potassium phosphate (175 mg, 0.83 mmol), tetrahydrofuran (2.5 mL) and water (0.5 mL). After degassed and purged with nitrogen several times, the microwave the tube was sealed.

The reaction mixture was stirred at 65° C. overnight. The reaction mixture was concentrated to dryness and the crude was purified by flash column chromatography (mobile phase: DCM: MeOH=10:1/DCM, 0-100%) to give compound 13-b (230 mg, 97%). LC-MS (ESI): m/z 860.2 (M+H)⁺.

### Synthesis of Compound 13-a

To a solution of 13-b (230 mg, 0.27 mmol) in DMF (6 mL) was added cesium fluoride (203 mg, 1.34 mmol) and the reaction mixture was stirred at room temperature overnight. Water was added to the reaction mixture and the aqueous phase was extracted with ethyl acetate (50 mL). The organic phase was washed twice with brine, dried over anhydrous sodium sulfate, filtered, and the organic phase was concentrated to dryness to give compound 13-a (172 mg, 91%). LC-MS (ESI): m/z 703.9 (M+H)⁺.

### Synthesis of Compounds 13 and 14

To a solution of 13-a (100 mg, 0.14 mmol) in acetonitrile (15 mL) was added a HCl/1,4-dioxane (4 M, 2.5 mL) in an ice-water bath. The reaction mixture was stirred at this temperature for 1 hour. The reaction mixture was warmed to room temperature and stirred for another 3 hours. It was neutralized by addition of saturated aqueous sodium bicarbonate solution, extracted with ethyl acetate (50 mL). The organic phase was dried over anhydrous sodium sulfate, filtered, the filtrate was concentrated to dryness and the crude was purified by prep-HPLC to give compound 13 (19 mg, 24%) and 14 (12.2 mg, 13%). Compound 13: LC-MS (ESI): m/z 559.3 (M+H)⁺;¹H NMR (400 MHz, CDCl₃): δ 10.15 (1H, s), 7.54 (1H, dd, J=9.6, 5.6 Hz), 7.24 (1H, s), 7.09 (1H, t, J=8.8 Hz), 7.02 (1H, s), 4.61-4.50 (1H, m), 4.50-4.41 (1H, d, J=10.8 Hz), 4.18 (1H, d, J=10.4 Hz), 4.08-3.92 (2H, m), 3.19-3.02 (2H, m), 2.77 (1H, s), 2.74-2.65 (1H, m), 2.39 (6H, s), 1.97-1.91 (3H, m), 1.76-1.66 (5H, m), 0.73 (2H, s), 0.56 (2H, dd, J=16.0, 9.6 Hz); compound 14: LC-MS (ESI): m/z 659.3 (M+H)⁺.

### Example 14 Synthetic Route of Compound 15

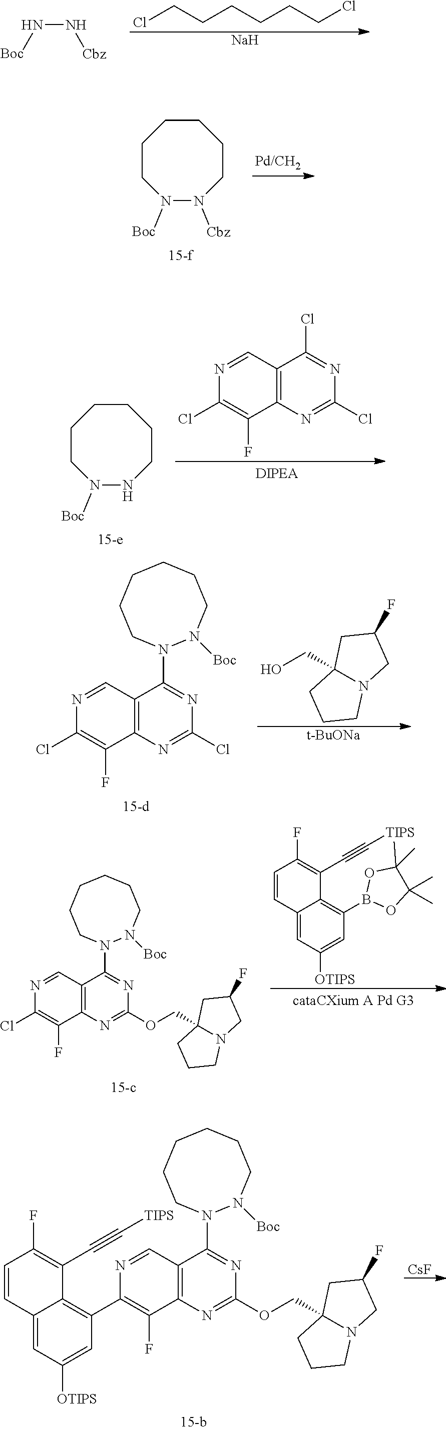

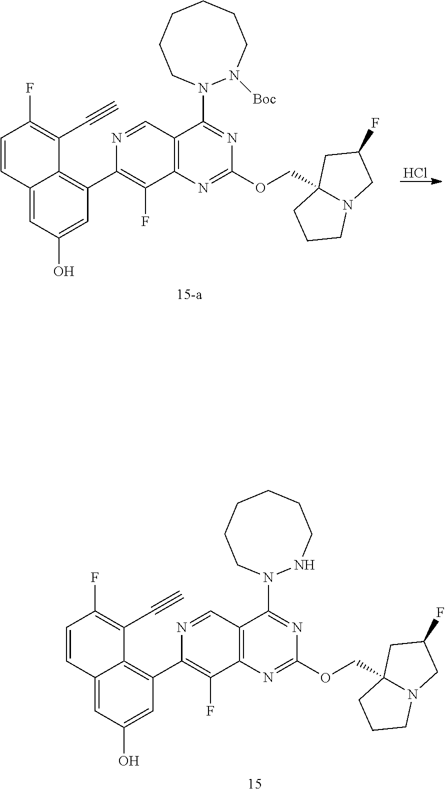 [SMILES: C#Cc1c(F)ccc2cc(O)cc(-c3ncc4c(N5CCCCCCN5C(=O)OC(C)(C)C)nc(OC[C@@]56CCCN5C[C@H](F)C6)nc4c3F)c12 ; Cl ; C#Cc1c(F)ccc2cc(O)cc(-c3ncc4c(N5CCCCCCN5)nc(OC[C@@]56CCCN5C[C@H](F)C6)nc4c3F)c12]

### Synthesis of Compound 15-f

Sodium hydride (60%, 0.75 g, 18.78 mmol) was added in batches to a solution of 2-(tert-butyl)hydrazine-1,2-dicarboxylic acid-1-benzyl ester (2 g, 7.51 mmol) in DMF (60 mL) in an ice-water bath. The mixture was stirred for 30 min at the same temperature, then at room temperature for 1 h, and then cooled down to 0° C. in an ice-water bath, and slowly added 1,6 -Dichlorohexane (1.16 g, 7.51 mmol). After addition, the mixture was slowly warmed to room temperature and stirred overnight. Saturated aqueous ammonium chloride was added to the reaction mixture and the aqueous phase was extracted with ethyl acetate (30 mL*2), the combined organic phases were washed twice with water and twice with brine. The organic phases were dried over anhydrous sodium sulfate, filtered, concentrated to dryness, and the crude product was purified by flash column chromatography (mobile phase: ethyl acetate/petroleum ether, 0-10%) to obtain 15-f (0.86 g, 33%).

LC-MS (ESI): m/z 249.3 (M−100+H)⁺.

### Synthesis of Compound 15-e

To a solution of 15-f (860 mg, 2.47 mmol) in ethyl acetate (25 mL) was added Pd/C (10%, 200 mg, 1.88 mmol) at room temperature, and the reaction mixture was degassed and purged with a hydrogen balloon several times, then was stirred at room temperature overnight. The reaction mixture was filtered to remove the Pd/C and the filtrate was concentrated to dryness to give compound 15-e (530 mg, 100.00%). LC-MS (ESI): m/z 159.2 (M−56+H)⁺.

### Synthesis of Compound 15-d

To a solution of 2,4,7-trichloro-8-fluoropyrido[4,3-d]pyrimidine (100 mg, 0.396 mmol) in dichloromethane (20 mL) was added 15-e (93 mg, 0.44 mmol) and N,N-diisopropylethylamine (154 mg, 1.19 mmol) in an acetone-dry ice-water bath and the mixture was stirred for 2 h at this temperature. Water was added to the reaction mixture and the aqueous phase was extracted with dichloromethane (30 mL). The organic phase was dried over anhydrous sodium sulfate, filtered, and the filtrate was concentrated to dryness and the crude was purified by flash column chromatography (mobile phase: ethyl acetate/petroleum ether, 0-10%) to give compound 15-d (169 mg, 99%). LC-MS (ESI): m/z 430.4 (M+H)⁺.

### Synthesis of Compound 15-c

To a solution of 15-d (169 mg, 0.39 mmol) in dichloromethane (15 mL) was added ((2R,7aS)-2-fluorohexahydro-1H-pyrrolizin-7a-yl)methanol (188 mg, 1.18 mmol) in an ice-water bath, followed by slow addition of sodium tert-butoxide (151 mg, 1.57 mmol), and the mixture was stirred at this temperature for 5 hours. Then water was added and the aqueous phase was extracted with dichloromethane (30 mL). The organic phase was dried over anhydrous sodium sulfate, filtered, and the filtrate was concentrated to dryness and the crude was purified by flash column chromatography (mobile phase: dichloromethane: methanol=10:1/dichloromethane, 0-50%) to give compound 15-c (154 mg, 70%). LC-MS (ESI): m/z 553.6 (M+H)⁺.

### Synthesis of Compound 15-b

To a 10 mL of microwave tube were added 15-c (154 mg, 0.28 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (209 mg, 0.334 mmol), methanesulfonic acid [n-butylbis(1-adamantyl)phosphine](2-amino-1,1′-biphenyl-2-yl)palladium(II) (20 mg, 0.028 mmol), potassium phosphate (177 mg, 0.84 mmol), tetrahydrofuran (2.5 mL) and water (0.5 mL). After degassed and purged with nitrogen several times, the microwave tube was sealed. The reaction mixture was stirred at 65° C. overnight. The reaction mixture was added water, and the aqueous phase was extracted with ethyl acetate (20 mL). The organic phase was dried over anhydrous sodium sulfate, filtered, and the filtrate was concentrated to dryness to obtain 15-b (280 mg, 99%). The crude product was used directly in the next step without purification.

### Synthesis of Compound 15-a

Cesium fluoride (209 mg, 1.38 mmol) was added to a solution of 15-b (280 mg, 0.28 mmol) in DMF (8 mL) at room temperature, and the reaction mixture was stirred at room temperature overnight.

The reaction mixture was added water, and the aqueous phase was extracted with ethyl acetate (30 mL). The organic phase was washed twice with brine, dried over anhydrous sodium sulfate, filtered. The organic phase was concentrated to dryness, and the crude was purified by flash column chromatography (mobile phase: dichloromethane: methanol=10:1/dichloromethane, 0-50%) to obtain 15-a (166 mg, 86%). LC-MS (ESI): m/z 703.8 (M+H)⁺.

### Synthesis of Compound 15

To a solution of 15-a (166 mg, 0.24 mmol) in acetonitrile (10 mL) was added HCl/1,4-dioxane (4M, 1.5 mL) at room temperature. The reaction mixture was stirred at room temperature overnight. It was neutralized by addition of saturated aqueous sodium bicarbonate solution, extracted with ethyl acetate (100 mL). The organic phase was dried over anhydrous sodium sulfate, filtered, the filtrate was concentrated to dryness and the crude was purified by prep-HPLC to give compound 15 (69 mg, 49%). LC-MS (ESI): m/z 603.3 (M+H)⁺; ¹H NMR (400 MHz, CDCl₃): δ 10.14 (1H, d, J=35.6 Hz), 7.54-7.38 (1H, m), 7.25 (1H, d, J=46.4,Hz), 7.14-7.00 (2H, m), 5.29 (1H, dd, J=53.6, 13.2 Hz), 4.62 (1H, s), 4.25-4.11 (2H, m), 4.10-3.77 (2H, m), 3.34-3.06 (5H, m), 3.02-2.93 (1H, m), 2.75 (1H, s), 2.43-2.04 (5H, m), 2.02-1.86 (4H, m), 1.73-1.40 (6H, m).

### Example 15 Synthetic Route of Compound 16 and 17

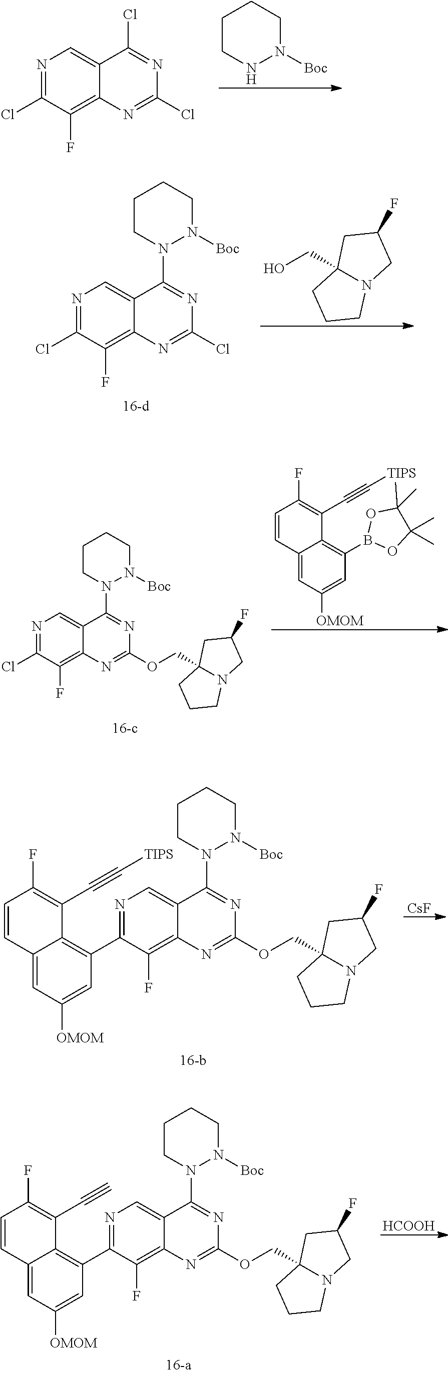

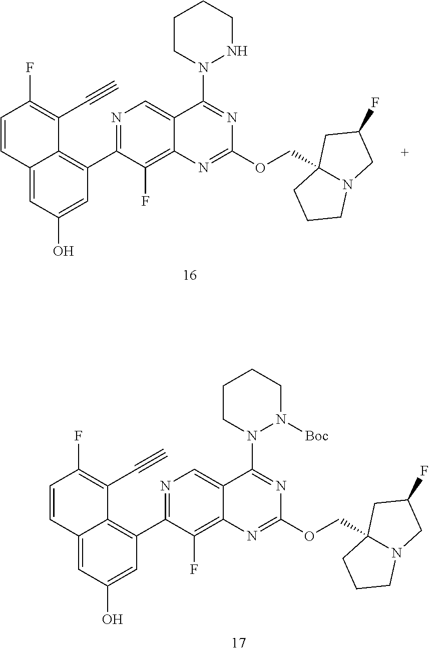 [SMILES: C#Cc1c(F)ccc2cc(O)cc(-c3ncc4c(N5CCCCN5)nc(OC[C@@]56CCCN5C[C@H](F)C6)nc4c3F)c12 ; C#Cc1c(F)ccc2cc(O)cc(-c3ncc4c(N5CCCCN5C(=O)OC(C)(C)C)nc(OC[C@@]56CCCN5C[C@H](F)C6)nc4c3F)c12]

### Synthesis of Compound 16-d

A reaction vial charged with tert-butyl tetrahydropyridazine-1(2H)-carboxylate (148 mg, 0.792 mmol), dichloromethane (30 mL), diisopropylethylamine (0.393 mL, 2.377 mmol), and 2,4,7-trichloro-8-fluoropyrido[4,3-d]pyrimidine (200 mg, 0.792 mmol) was cooled in a dry ice-acetone bath. The reaction mixture was stirred under protection of nitrogen for 1 hour. The reaction mixture was quenched with water and the aqueous phase was extracted with dichloromethane (50 mL*2).

The organic phase was dried, concentrated to dryness and the residue was purified by column chromatography (mobile phase: ethyl acetate/petroleum ether 0/100 to 10/90) to afford compound 16-d (270 mg, 85%). LC-MS (ESI): m/z=402.1 (M+H)⁺.

### Synthesis of Compound 16-c

A reaction vial charged with 16-d (100 mg, 0.249 mmol), dichloromethane (30 mL), ((2R,7aS)-2-fluorohexahydro-1H-pyrrolozin-7a-yl)methanol (47 mg, 0.298 mmol) was added sodium tert-butoxide (60 mg, 0.622 mmol) in an ice-water bath. After addition, the reaction mixture was stirred in an ice-water bath for half an hour, supplemented with sodium tert-butanol (60 mg, 0.622 mmol), and continued stirring for half an hour; then supplemented with sodium tert-butanol (60 mg, 0.622 mmol), and continued stirring for half an hour; and supplemented again with sodium tert-butanol (60 mg, 0.622 mmol), and continued stirring for half an hour. The reaction mixture was quenched with water and the aqueous phase was extracted with dichloromethane (50 mL*2). The organic phase was dried over anhydrous sodium sulfate, concentrated to dryness, and the residue was purified by column chromatography (mobile phase: ammonia methanol/dichloromethane 0/100 to 6/94) to give compound 16-c (114 mg, 87%). LC-MS (ESI): m/z=525.8(M+H)⁺.

### Synthesis of Compound 16-b

To a microwave tube were added 16-c (114 mg, 0.217 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (167 mg, 0.326 mmol), tetrahydrofuran (15 mL), water (1.5 mL), potassium phosphate (138 mg, 0.651 mmol), cataCXium A Pd G3 (16 mg, 0.022 mmol). After degassed and purged with nitrogen for three times, the microwave tube was sealed, and the reaction mixture was stirred at 65° C. overnight. The next day, the reaction mixture was concentrated to dryness and the residue was purified by column chromatography (mobile phase: methanol/dichloromethane 0/100 to 2/98) to give compound 16-b (198 mg, 100%). LC-MS (ESI): m/z=875.7 (M+H)⁺.

### Synthesis of Compound 16-a

To a reaction flask were added 16-b (198 mg, 0.226 mmol), DMF (10 mL), cesium fluoride (343 mg, 2.26 mmol), and the reaction mixture was stirred for 1 h at room temperature under atmosphere of nitrogen. The reaction mixture was quenched with water and the aqueous phase was extracted with ethyl acetate (50 mL*2). The organic phase was washed with brine (50 mL*3), dried over anhydrous sodium sulfate, concentrated to dryness, and the residue was purified by column chromatography (mobile phase: methanol/dichloromethane 0/100 to 5/95) to give compound 16-a (127 mg, 78%). LC-MS (ESI): m/z=719.9 (M+H)⁺.

### Synthesis of Compounds 16 and 17

A reaction vial charged with 16-a (127 mg, 0.177 mmol) and formic acid (10 mL) was stirred at room temperature for 10 hours. The reaction mixture was concentrated to dryness, purified by prep-HPLC (ammonium bicarbonate) and lyophilized to give compounds 16 (25 mg, 25%) and 17 (5 mg, 4%). Compound 16, LC-MS (ESI): m/z=575.2 (M+H)⁺; ¹H NMR (400M, DMSO-d₆) δ 10.29 (1H, s), 10.14 (1H, s), 8.00-7.92 (1H, m), 7.46 (1H, t, J=8.8 Hz), 7.38 (1H, d, J=2.4 Hz), 7.16(1H, d, J=2.0 Hz), 5.63 (1H, t, J=6.4 Hz), 5.28 (1H, d, J=53.6 Hz), 4.29-3.95 (4H, m), 3.92 (1H, s), 3.15-2.96 (5H, m), 2.88-2.76 (1H, m), 2.16-1.65(10H, m). Compound 17, LC-MS (ESI): m/z=675.3 (M+H)⁺.

### Example 16 Synthetic Route of Compound 18

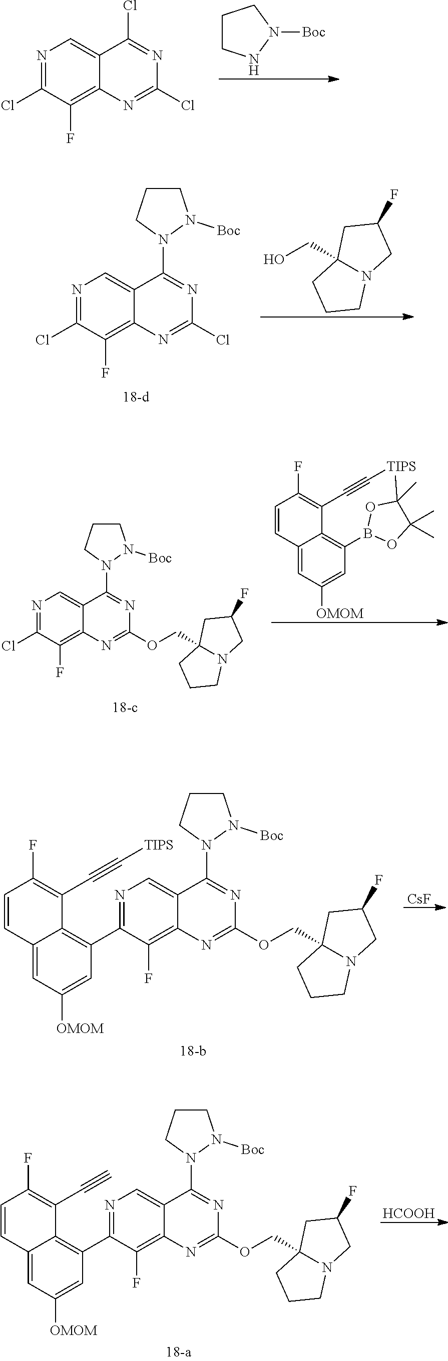

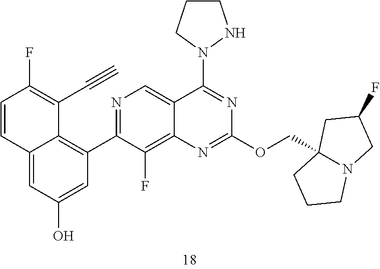 [SMILES: C#Cc1c(F)ccc2cc(O)cc(-c3ncc4c(N5CCCN5)nc(OC[C@@]56CCCN5C[C@H](F)C6)nc4c3F)c12]

### Synthesis of Compound 18-d

A reaction vial charged with tert-butyl pyrazolidine-1-carboxylate (136 mg, 0.79 mmol), dichloromethane (30 mL), diisopropylethylamine (0.393 mL, 2.38 mmol) was cooled in a dry ice-acetone bath and added 2,4,7-trichloro-8-fluoropyrido[4,3-d]pyrimidine (200 mg, 0.79 mmol). The reaction mixture was stirred under protection of nitrogen for 1 hour, then was quenched with water and the aqueous phase was extracted with dichloromethane (50 mL*2). The organic phase was dried and concentrated to dryness to give compound 18-d (307 mg, 100%), which was used without purification in the next step. LC-MS (ESI): m/z=388.1 (M+H)⁺.

### Synthesis of Compound 18-c

A reaction vial charged with 18-d (307 mg, 0.79 mmol), dichloromethane (30 mL), ((2R,7aS)-2-fluorohexahydro-1H-pyrrolozin-7a-yl)methanol (126 mg, 0.79 mmol) was added sodium tert-butoxide (190 mg, 1.977 mmol) in an ice-water bath, and the reaction mixture was stirred for 1 hour. Supplemented with sodium tert-butanol (190 mg, 1.977 mmol) and continued stirring for 1 hour. The reaction mixture was quenched with water and the aqueous phase was extracted with dichloromethane (80 mL*2). The organic phase was dried over anhydrous sodium sulfate, concentrated to dryness, and the residue was purified by column chromatography (mobile phase: methanol/dichloromethane 0/100 to 3/97) to give compound 18-c (217 mg, 54%). LC-MS (ESI): m/z=511.5(M+H)⁺.

### Synthesis of Compound 18-b

To a microwave tube were added 18-c (217 mg, 0.42 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (326 mg, 0.637 mmol), tetrahydrofuran (15 mL), water (1.5 mL), potassium phosphate (270 mg, 1.274 mmol), cataCXium A Pd G3 (31 mg, 0.042 mmol). After degassed and purged with nitrogen for three times, the microwave tube was sealed and stirred at 65° C. overnight. The next day, the reaction mixture was concentrated to dryness and the residue was purified by column chromatography (mobile phase: methanol/dichloromethane 0/100 to 4/96) to give compound 18-b (396 mg, 100%). LC-MS (ESI): m/z=861.7 (M+H)⁺.

### Synthesis of Compound 18-a

To a reaction flask were added 18-b (396 mg, 0.46 mmol), DMF (10 mL), cesium fluoride (699 mg, 4.599 mmol) and the reaction mixture stirred for 1 h at room temperature under protection of nitrogen, then was quenched with water and the aqueous phase was extracted with ethyl acetate (50 mL*2). The organic phase was washed with brine (50 mL*3), dried over anhydrous sodium sulfate, concentrated to dryness, and the residue was purified by column chromatography (mobile phase: methanol/dichloromethane 0/100 to 5/95) to give compound 18-a (236 mg, 73%). LC-MS (ESI): m/z=705 (M+H)⁺.

### Synthesis of Compound 18

A reaction vial charged with 18-a (100 mg, 0.14 mmol) and formic acid (10 mL) was stirred at room temperature overnight. The following day, the reaction mixture was concentrated to dryness, purified by prep-HPLC (ammonium bicarbonate) and lyophilized to give compound 18 (19 mg, 24%). LC-MS (ESI): m/z=561.3 (M+H)⁺; ¹H NMR (400M, DMSO-d₆): δ 10.19 (1H, d, J=9.2 Hz), 10.04 (1H, d, J=44.8 Hz), 8.03-7.92 (1H, m), 7.76-6.05 (1H, m), 7.46 (1H, t, J=8.8 Hz), 7.41-7.35 (1H, m), 7.20-7.10 (1H, m), 5.28 (1H, d, J=55.2 Hz), 4.23-3.96 (3H, m), 3.92(1H, d, J=8.0 Hz), 3.83 (1H, t, J=7.2 Hz), 3.23-2.98 (5H, m), 2.88-2.78 (1H, m), 2.21-1.70 (8H, m).

### Example 17 Synthetic Route of Compound 19

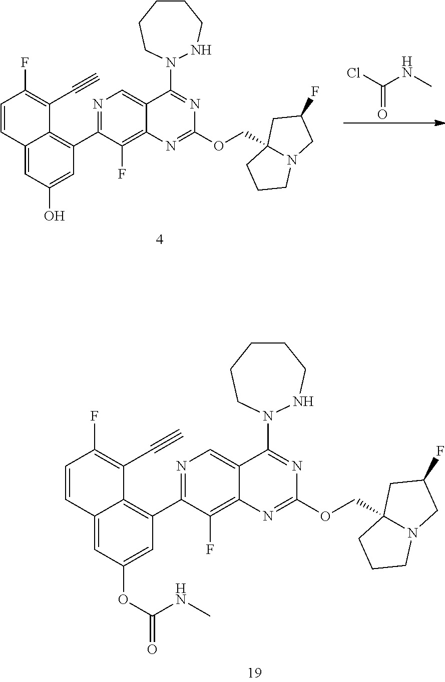

### Synthesis of Compound 19

A reaction vial charged with compound 4 (40 mg, 0.068 mmol), methylene chloride (15 mL) and diisopropylethylamine (56 uL, 0.34 mmol) was added methylamine carbonyl chloride (14 mg, 0.134 mmol) in an ice-water bath. After addition, the reaction mixture was stirred at room temperature overnight. The next day, the reaction mixture was quenched with saturated sodium bicarbonate solution, extracted with dichloromethane (50 mL*2), dried over anhydrous sodium sulfate, concentrated to dryness, purified by prep-HPLC (trifluoroacetic acid), lyophilized to give compound 19 (26 mg, 59%). LC-MS (ESI): m/z=646.3 (M+H)⁺.

### Example 18 Synthetic Route of Compound 20

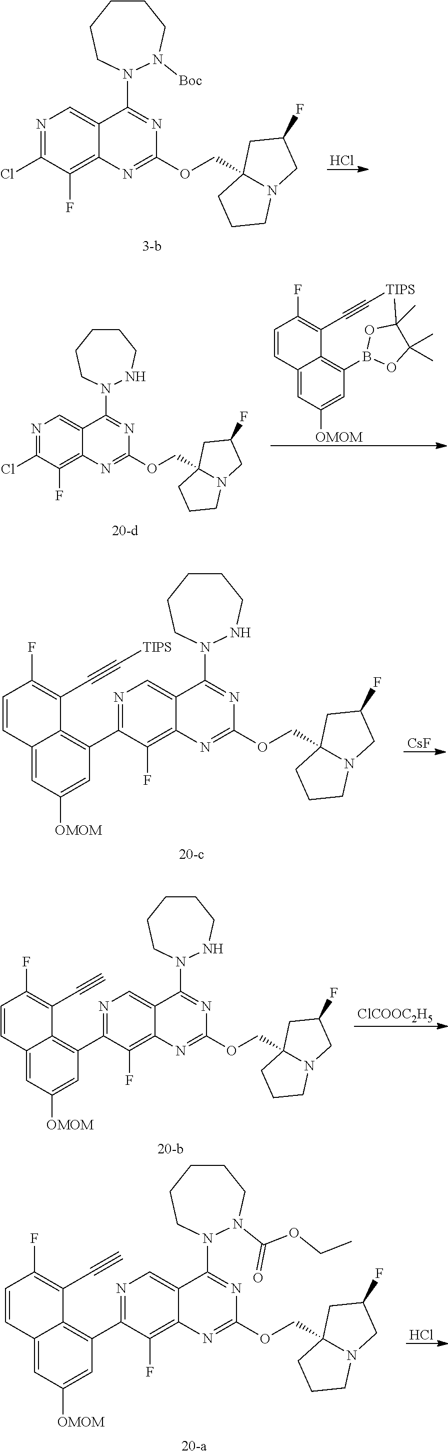

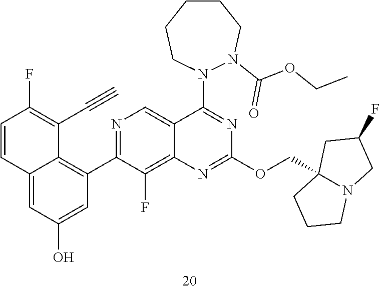 [SMILES: C#Cc1c(F)ccc2cc(O)cc(-c3ncc4c(N5CCCCCN5C(=O)OCC)nc(OC[C@@]56CCCN5C[C@H](F)C6)nc4c3F)c12]

### Synthesis of Compound 20-d

To a reaction flask were added 3-b (290 mg, 0.54 mmol), acetonitrile (20 mL), hydrochloride/1,4-dioxane (4 M, 2 mL), and the reaction mixture was stirred at room temperature overnight under protection of nitrogen. The next day, the reaction mixture was concentrated to dryness, and the residue was added 20 mL of methanol, adjusted to pH 7 with saturated sodium bicarbonate solution.

The solvents were removed, and the residue was redissolved in dichloromethane, dried over anhydrous sodium sulfate, filtered, and the filtrate was concentrated to dryness to give compound 20-d (288 mg, 100%), which was used directly in the next step without purification. LC-MS (ESI): m/z=439.6 (M+H)⁺.

### Synthesis of Compound 20-c

To a microwave tube were added 20-d (280 mg, 0.64 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (490 mg, 0.957 mmol), tetrahydrofuran (12 mL), water (1.2 mL), potassium phosphate (406 mg, 1.914 mmol), cataCXium A Pd G3 (93 mg, 0.128 mmol). After degassed and purged with nitrogen for three times, the microwave tube was sealed and stirred at 65° C. overnight. The next day, the reaction mixture was concentrated to dryness and the residue was purified by column chromatography (mobile phase: methanol/dichloromethane 0/100 to 10/90) to give compound 20-c (380 mg, 75%). LC-MS (ESI): m/z=789.9 (M+H)⁺.

### Synthesis of Compound 20-b

To a reaction flask were added 20-c (380 mg, 0.48 mmol), DMF (10 mL), cesium fluoride (732 mg, 4.816 mmol) and the reaction mixture was stirred for 1 h at room temperature under protection of nitrogen. The reaction mixture was quenched with water and the aqueous phase was extracted with ethyl acetate (50 mL*2). The organic phase was washed with brine (50 mL*3), dried over anhydrous sodium sulfate and concentrated to dryness to give compound 20-b (323 mg, 100%), which was used directly in the next step without purification. LC-MS (ESI): m/z=633.8 (M+H)⁺.

### Synthesis of Compound 20-a

A reaction vial charged with 20-b (50 mg, 0.079 mmol), dichloromethane (20 mL) and DIPEA (0.131 mL, 0.79 mmol) was added ethyl chloroformate (75 uL, 0.79 mmol) dropwise with stirring at room temperature and stirred for half an hour at room temperature under protection of nitrogen.

The reaction mixture was quenched with saturated sodium bicarbonate solution and the aqueous phase was extracted with dichloromethane (50 mL*2). The organic phase was concentrated to dryness and the residue was purified by column chromatography (mobile phase: ethyl acetate/petroleum ether 0/100 to 50/50) to give compound 20-a (50 mg, 90%). LC-MS (ESI): m/z=705.4 (M+H)⁺.

### Synthesis of Compound 20

A reaction vial charged with 20-a (50 mg, 0.071 mmol), acetonitrile (10 mL) and hydrochloride/1,4-dioxane (4M, 1 mL, 4 mmol) was stirred at room temperature for 2 hours. The reaction mixture was concentrated to dryness at room temperature, and the residue was added methanol (20 mL) and adjusted to pH 7 with saturated sodium bicarbonate solution. The mixture was concentrated to dryness, purified by column chromatography (mobile phase: methanol/dichloromethane 0/100 to 6/94), purified by prep-HPLC (ammonium bicarbonate) and lyophilized to give compound 20 (6 mg, 13%). LC-MS (ESI): m/z=661.3 (M+H)⁺.

### Example 19 Synthetic Route of Compound 21

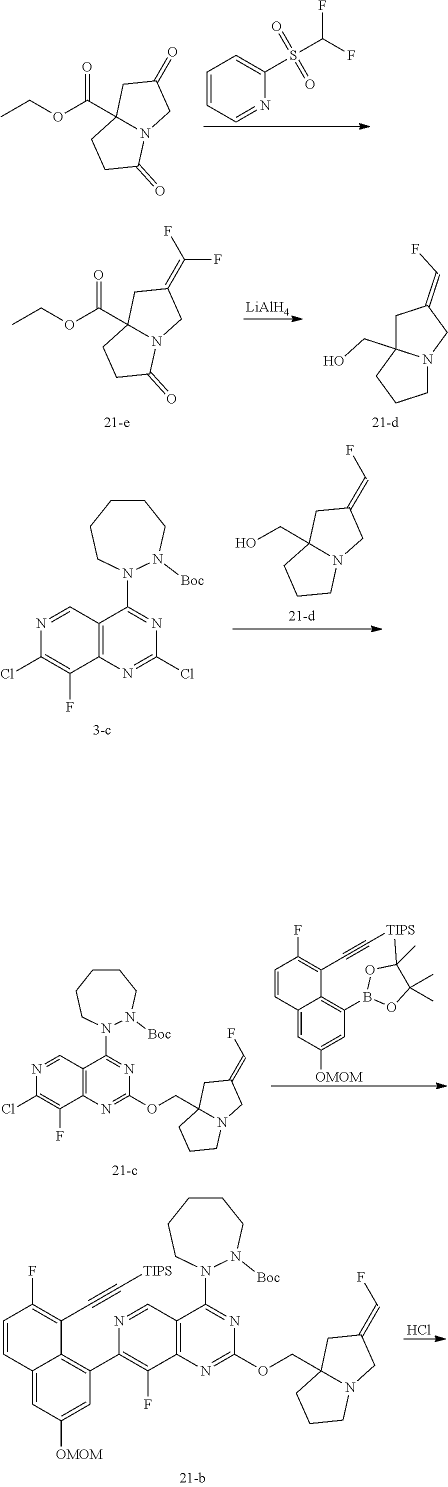

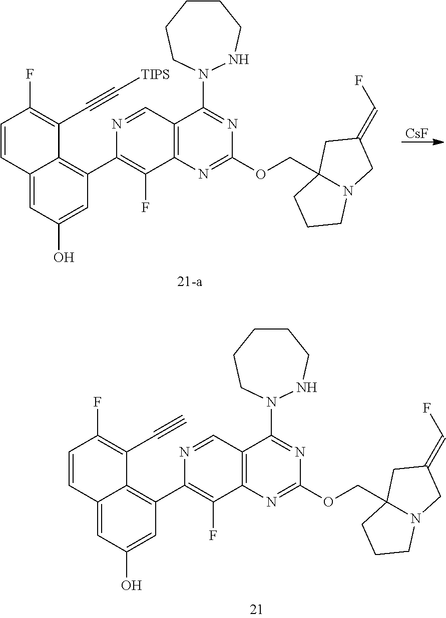

### Synthesis of Compound 21-e

Difluoromethyl (2-pyridyl) sulfone (4118 mg, 21.32 mmol) and DMF (50 mL) were added to a reaction flask at room temperature. After degassed and purged with nitrogen, the above mixture was cooled down to −50° C., was added a solution of ethyl 2,5-dioxohexahydro-1H-pyrrolizine-7a-carboxylate (5000 mg, 23.67 mmol) in DMF (50 mL) dropwise while the temperature was controlled to be no higher than −45° C. After addition, the reaction mixture was naturally warmed to room temperature for 1 hour. The reaction mixture was added saturated aqueous ammonium chloride solution (30 mL) to quench the reaction, then was added concentrated hydrochloric acid (37% wt, 18 mL) and stirred at room temperature for 1 hour. The resulting mixture was added water, and the aqueous phase was extracted with ethyl acetate. The organic phases were combined, dried over anhydrous sodium sulfate, filtered, concentrated, and the residue was purified by column chromatography (mobile phase: PE/DCM 10/0 to 0/10) to give compound 21-e (1000 mg, 19%).

LC-MS (ESI): m/z 246.2 (M+H)⁺.

### Synthesis of Compound 21-d

A solution of lithium aluminum hydride (1M, 6.52 mL) in tetrahydrofuran was added to a reaction flask at room temperature. After degassed and purged with nitrogen, the above mixture was cooled down to 0° C., was added a solution of 21-e (800 mg, 3.26 mmol) in THF (5 mL) dropwise. After addition, the reaction mixture was stirred at 65° C. for 3 hours. The reaction mixture was cooled to room temperature and quenched by the addition of methanol (30 mL) under protection of nitrogen, then about 3 g of sodium sulfate decahydrate was added and the mixture was stirred at room temperature for 2 hours. The mixture was filtered and the filter cake was washed with methanol. The filtrates were combined, concentrated and the residue was purified by column chromatography (mobile phase: DCM/MeOH 10/0 to 10/1) to give compound 21-d (130 mg, 23%). LC-MS (ESI): m/z 172.2 (M+H)⁺.

### Synthesis of Compound 21-c

3-c (379 mg, 0.91 mmol), 21-d (130 mg, 0.76 mmol), 1,4-dioxane (10 mL) and DIPEA (294 mg, 2.27 mmol) were added to a reaction vial at room temperature. After degassed and purged with nitrogen, the reaction mixture was stirred at 90° C. overnight. The reaction mixture was concentrated and the residue was purified by column chromatography (mobile phase: DCM/MeOH 10/0 to 10/1) to give compound 21-c (140 mg, 33%). LC-MS (ESI): m/z 551.8 (M+H)⁺.

### Synthesis of Compound 21-b

At room temperature, 21-c (140 mg, 0.25 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (156 mg, 0.30 mmol), cataCXium A Pd G3 (19 mg, 0.03 mmol), potassium phosphate (162 mg, 0.76 mmol), THF (2 mL) and water (0.5 mL) were combined in a microwave tube. The mixture was degassed and purged with nitrogen, sealed, and stirred at 65° C. overnight. The reaction mixture was concentrated and the residue was purified by column chromatography (mobile phase: DCM/MeOH 10/0 to 10/1) to give compound 21-b (90 mg, 39%). LC-MS (ESI): m/z 902.0 (M+H)⁺.

### Synthesis of Compound 21-a

At room temperature, 21-b (90 mg, 0.10 mmol) and acetonitrile (5 mL) were added to a reaction flask. After degassed and purged with nitrogen, the above mixture was cooled down to 0° C., the above mixture was added HCl/1,4-dioxane (4.0 M, 1 mL). The reaction mixture was stirred at 0° C. for 3 hours. The reaction mixture was concentrated at low temperature to give the crude compound 21-a (130 mg). LC-MS (ESI): m/z 757.5 (M+H)⁺.

### Synthesis of Compound 21

To a reaction flask, crude compound 21-a (130 mg, about 0.10 mmol), DMF (3 mL) and cesium fluoride (76 mg, 0.50 mmol) were added at room temperature. After degassed and purged with nitrogen, the reaction mixture was stirred at 60° C. for 1 hour. The reaction mixture was filtered and the filtrate was purified by prep-HPLC to give compound 21 (4 mg, 7%). LC-MS (ESI): m/z 601.6 (M+H)⁺.

### Example 20 Synthetic Route of Compound 22

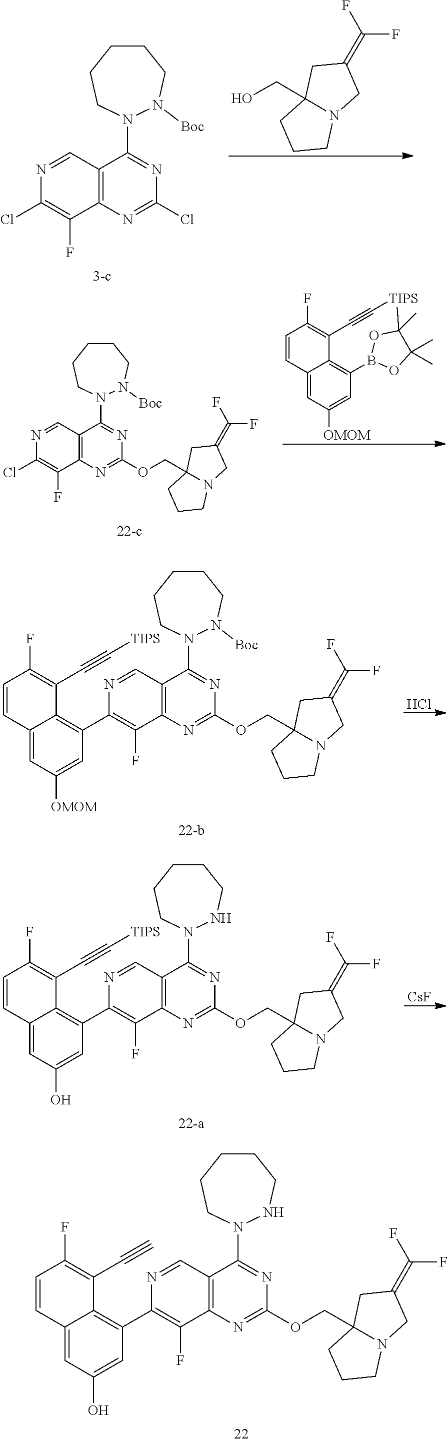

### Synthesis of Compound 22-c

At room temperature, 3-c (200 mg, 0.48 mmol), (2-(difluoromethylene)tetrahydro-1H-pyrrolizin-7A(5H)-yl)methanol (118 mg, 0.62 mmol), 1,4-dioxane (10 mL) and DIPEA (186 mg, 1.44 mmol) were added to a reaction vial. After degassed and purged with nitrogen, the reaction mixture was stirred at 90° C. overnight. The reaction mixture was concentrated and the residue was purified by column chromatography (mobile phase: DCM/MeOH 10/0 to 10/1) to give compound 22-c (140 mg, 51%). LC-MS (ESI): m/z 569.8 (M+H)⁺.

### Synthesis of Compound 22-b

At room temperature, 22-c (140 mg, 0.25 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (164 mg, 0.32 mmol), cataCXium A Pd G3 (18 mg, 0.03 mmol), potassium phosphate (157 mg, 0.74 mmol), THF (2 mL) and water (0.5 mL) were combined in a microwave tube. After degassed and purged with nitrogen, the reaction mixture was stirred at 65° C. overnight. The reaction mixture was concentrated and the residue was purified by column chromatography (mobile phase: DCM/MeOH 10/0 to 10/1) to afford compound 22-b (190 mg, 84%). LC-MS (ESI): m/z 919.7 (M+H)⁺.

### Synthesis of Compound 22-a

At room temperature, 22-b (190 mg, 0.21 mmol) and acetonitrile (5 mL) were added to a reaction flask. After degassed and purged with nitrogen, the above mixture was cooled down to 0° C., and was added HCl/1,4-dioxane (4.0 M, 1 mL). The reaction mixture was stirred at 0° C. for 3 hours. The reaction mixture was concentrated at low temperature to give the crude compound 22-a (230 mg).

LC-MS (ESI): m/z 775.5 (M+H)⁺.

### Synthesis of Compound 22

To a reaction flask, crude compound 22-a (230 mg, 0.21 mmol), DMF (3 mL) and cesium fluoride (159 mg, 1.05 mmol) were added at room temperature. After degassed and purged with nitrogen, the reaction mixture was stirred at 60° C. for 1 hour. The reaction mixture was filtered and the filtrate was purified by prep-HPLC to give compound 22 (23 mg, 18%). LC-MS (ESI): m/z 619.5 (M+H)⁺.

### Synthetic Route of Comparative Compound 1′

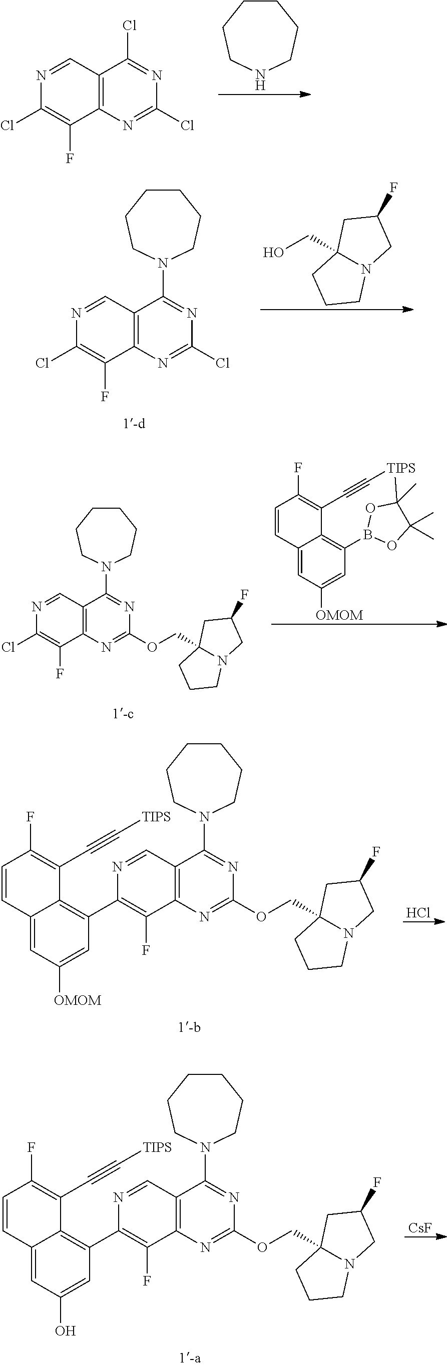

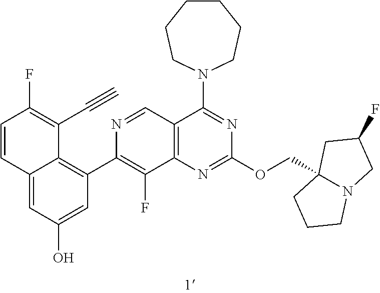 [SMILES: C#Cc1c(F)ccc2cc(O)cc(-c3ncc4c(N5CCCCCC5)nc(OC[C@@]56CCCN5C[C@H](F)C6)nc4c3F)c12]

### Synthesis of Compound 1′-d

At room temperature, 2,4,7-trichloro-8-fluoropyrido[4,3-d]pyrimidine (400 mg, 1.58 mmol), DCM (20 mL) were added to a reaction vial. After degassed and purged with nitrogen, the above mixture was cooled to −78° C. in a dry ice-acetone bath and was added DIPEA (614 mg, 4.75 mmol) and hexamethyleneimine (141 mg, 1.42 mmol). After addition, the reaction mixture was naturally warmed to room temperature overnight. The reaction mixture was added with water, and the aqueous phase was extracted with DCM. The organic phases were combined, dried over anhydrous sodium sulfate, concentrated, and the residue was purified by column chromatography (mobile phase: PE/EA 10/0 to 6/4) to give compound 1′-d (390 mg, 78%). LC-MS (ESI): m/z 315.4 (M+H)⁺.

### Synthesis of Compound 1′-c

At room temperature, 1′-d (400 mg, 1.27 mmol), ((2R,7aS)-2-fluorohexahydro-1H-pyrrolizin-7a-yl)methanol (404 mg, 2.54 mmol), 1,4-dioxane (10 mL) and DIPEA (656 mg, 5.08 mmol) were added to a reaction vial. After degassed and purged with nitrogen, the reaction mixture was stirred at 90° C. overnight. The reaction mixture was concentrated and the residue was purified by column chromatography (mobile phase: DCM/MeOH 10/0 to 10/1) to give compound 1′-c (450 mg, 81%).

LC-MS (ESI): m/z 438.2 (M+H)⁺.

### Synthesis of Compound 1′-b

At room temperature, 1′-c (200 mg, 0.46 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (281 mg, 0.55 mmol), cataCXium A Pd G3 (33 mg, 0.05 mmol), potassium phosphate (291 mg, 1.37 mmol), THF (2 mL) and water (0.5 mL) were combined in a microwave tube. After degassed and purged with nitrogen, the reaction mixture was stirred at 65° C. overnight. The reaction mixture was concentrated and the residue was purified by column chromatography (mobile phase: DCM/MeOH 10/0 to 10/1) to give compound 1′-b (230 mg, 64%). LC-MS (ESI): m/z 789.0 (M+H)⁺.

### Synthesis of Compound 1′-a

At room temperature, 1′-b (230 mg, 0.29 mmol) and acetonitrile (5 mL) were added to a reaction flask. After degassed and purged with nitrogen, the above mixture was cooled down to 0° C., and was added HCl/1,4-dioxane (4.0 M, 1 mL). After addition, the reaction mixture was stirred at 0° C. for 3 hours. The reaction mixture was concentrated at low temperature to give the crude compound 1′-a (270 mg). LC-MS (ESI): m/z 745.0 (M+H)⁺.

### Synthesis of Comparative compound 1′

To a reaction flask, crude 1′-a (270 mg, 0.29 mmol), DMF (3 mL) and cesium fluoride (220 mg, 1.45 mmol) were added at room temperature. After degassed and purged with nitrogen, the reaction mixture was stirred at 60° C. for 1 hour. The reaction mixture was filtered and the filtrate was purified by prep-HPLC to give compound 1′ (69 mg, 40%). LC-MS (ESI): m/z 588.7 (M+H)⁺.

### Example 21 Synthetic Route of Compound 23

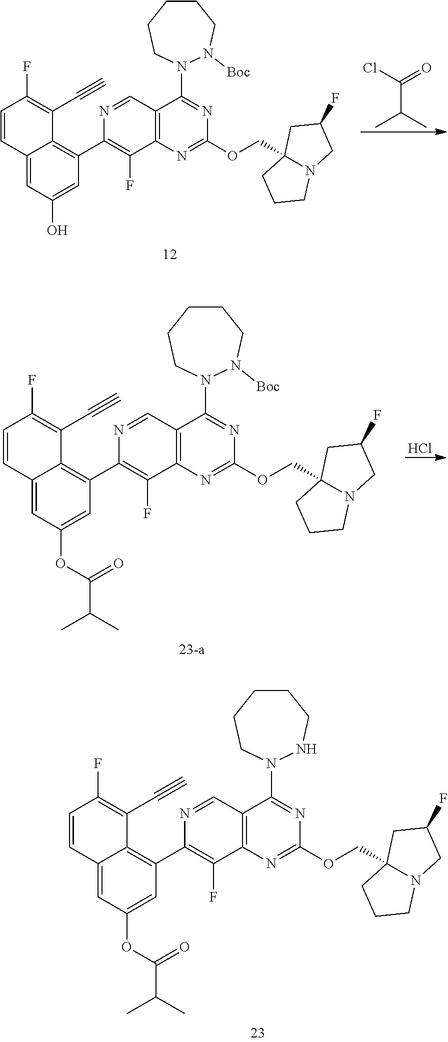

### Synthesis of Compound 23-a

To a solution of compound 12 (50 mg, 0.073 mmol) in dichloromethane (10 mL) was added isobutyryl chloride (16 mg, 0.146 mmol) and N,N-diisopropylethylamine (28 mg, 0.219 mmol) in an ice-water bath. The reaction mixture was stirred at this temperature for 1 hour. Water was added to the reaction mixture and the aqueous phase was extracted with dichloromethane (20 mL). The organic phase was dried over anhydrous sodium sulfate, filtered and concentrated to dryness to give compound 23-a (55 mg, 100%). The crude product was used directly in the next step without purification. LC-MS (ESI): m/z 759.9 (M+H)⁺.

### Synthesis of Compound 23

To a solution of 23-a (55 mg, 0.072 mmol) in acetonitrile (8 mL) was added HCl/1,4-dioxane (4 M, 1 mL) at room temperature and the reaction mixture was stirred at this temperature overnight. The reaction mixture was neutralized by adding saturated aqueous sodium bicarbonate solution and the aqueous phase was extracted with ethyl acetate (30 mL). The organic phase was dried over anhydrous sodium sulfate, filtered, and the filtrate was concentrated to dryness and the crude product was purified by prep-HPLC to give compound 23 (10.6 mg, 22%). LC-MS (ESI): m/z 659.3 (M+H)⁺.

### Example 22 Synthetic Route of Compound 24

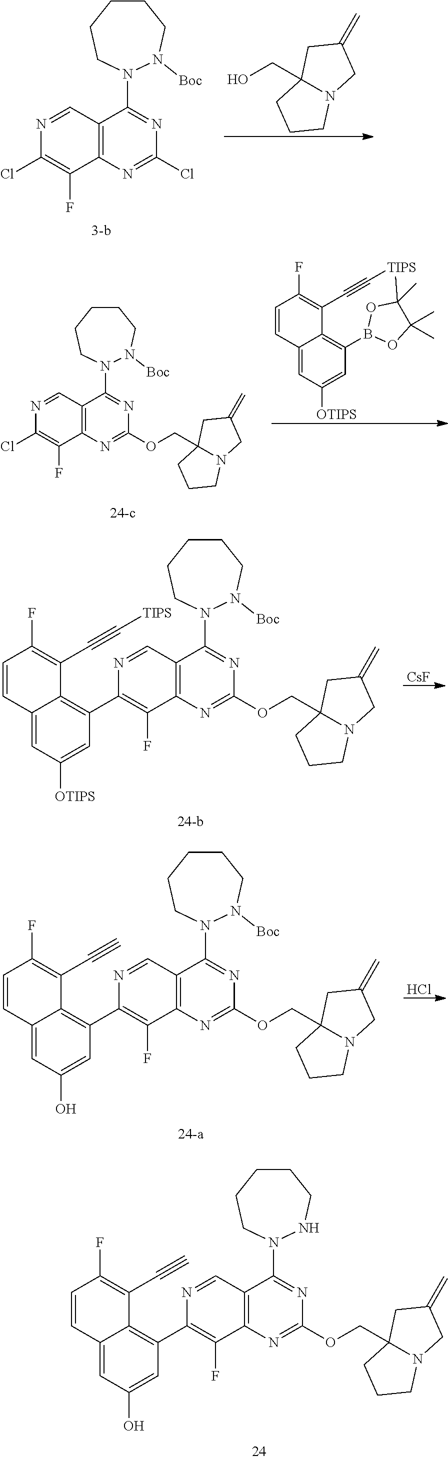

### Synthesis of Compound 24-c

To a solution of 3-b (160 mg, 0.38 mmol) in dichloromethane (15 mL) was added (2-methylenehexahydro-1H-pyrrolizin-7a-yl)methanol (88 mg, 0.58 mmol) in an ice-water bath, followed by slow addition of sodium tert-butoxide (111 mg, 1.15 mmol), and the mixture was stirred for 3 hours at this temperature. Water was added to the reaction mixture and the aqueous phase was extracted with dichloromethane (30 mL). The organic phase was dried over anhydrous sodium sulfate, filtered, and the filtrate was concentrated to dryness and the crude was purified by flash column chromatography (mobile phase: dichloromethane: methanol=10:1/dichloromethane, 0-50%) to give compound 24-c (93 mg, 45%). LC-MS (ESI): m/z 533.8 (M+H)⁺.

### Synthesis of Compound 24-b

To a 10 mL microwave tube were added 24-c (93 mg, 0.17 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (131 mg, 0.21 mmol), methanesulfonic acid [n-butylbis(1-adamantyl)phosphine](2-amino-1,1′-biphenyl-2-yl)palladium(II) (13 mg, 0.017 mmol), potassium phosphate (111 mg, 0.52 mmol), tetrahydrofuran (2.5 mL) and water (0.5 mL). After degassed and purged with nitrogen several times, the microwave tube was sealed. The reaction mixture was stirred at 65° C. overnight. Water was added to the reaction mixture and the aqueous phase was extracted with ethyl acetate (20 mL). The organic phase was dried over anhydrous sodium sulfate, filtered, and the filtrate was concentrated to dryness to give compound 24-b (170 mg, 98%). The crude product was used directly for the next step without purification. LC-MS (ESI): m/z 996.7 (M+H)⁺.

### Synthesis of Compound 24-a

Cesium fluoride (130 mg, 0.85 mmol) was added to a solution of 24-b (170 mg, 0.17 mmol) in N,N-dimethylformamide (8 mL) at room temperature and the mixture was stirred at room temperature overnight. Water was added to the reaction mixture and the aqueous phase was extracted with ethyl acetate (30 mL). The organic phase was washed twice with brine, dried over anhydrous sodium sulfate, filtered, and the filtrate was concentrated to dryness and the crude was purified by flash column chromatography (mobile phase: DCM: MeOH=10:1/DCM, 0-50%) to give compound 24-a (70 mg, 60%). LC-MS (ESI): m/z 683.4 (M+H )+.

### Synthesis of Compound 24

To a solution of 24-a (70 mg, 0.103 mmol) in acetonitrile (10 mL) was added hydrogen chloride/1,4-dioxane (4 M, 1 mL) at room temperature and the reaction mixture was stirred at this temperature overnight. The reaction mixture was added saturated aqueous sodium bicarbonate to neutralize, and the aqueous phase was extracted with ethyl acetate (20 mL). The organic phase was dried over anhydrous sodium sulfate, filtered, and the filtrate was concentrated to dryness and the crude was purified by prep-HPLC to give compound 24 (8 mg, 13%). LC-MS (ESI): m/z 583.3 (M+H)⁺.

### Example 23 Synthetic Route of Compound 25

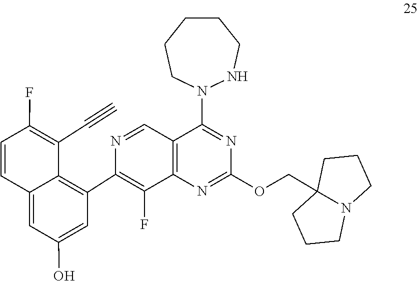 [SMILES: C#Cc1c(F)ccc2cc(O)cc(-c3ncc4c(N5CCCCCN5)nc(OCC56CCCN5CCC6)nc4c3F)c12]

Referring to synthetic route of compounds 3 and 4, compound 25 was obtained using hexahydro-1H-pyrrolizin-7a-yl methanol instead of ((2R,7aS)-2-fluorohexahydro-1H- pyrrolizin-7a-yl)methanol. LC-MS (ESI): m/z 571.2 (M+H)⁺.

### Example 24 Synthetic Route of Compound 26

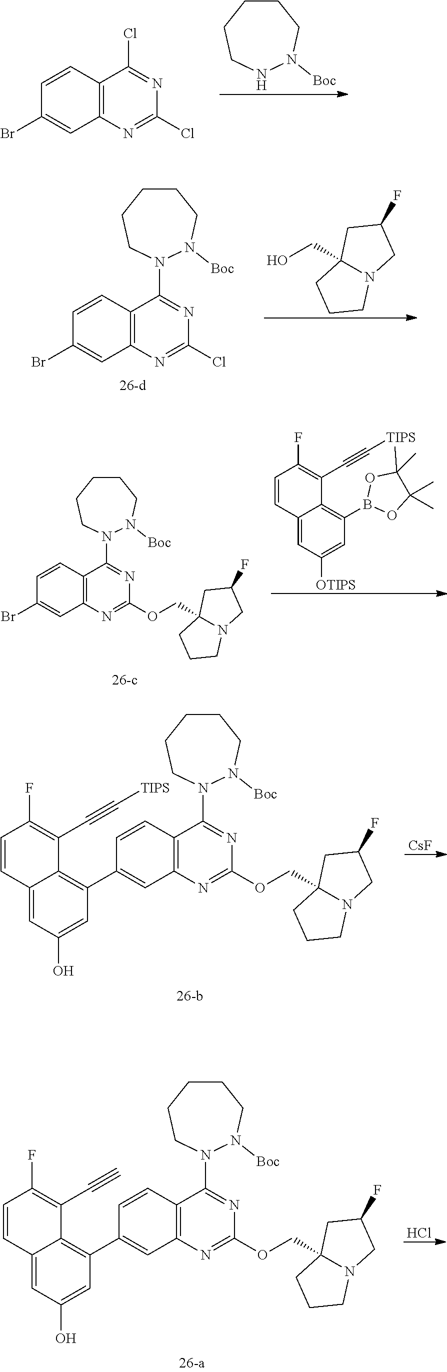

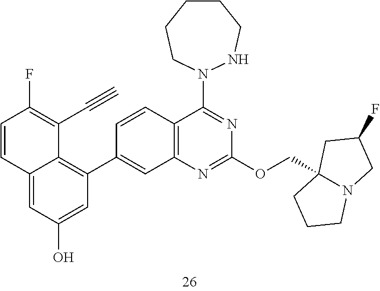 [SMILES: C#Cc1c(F)ccc2cc(O)cc(-c3ccc4c(N5CCCCCN5)nc(OC[C@@]56CCCN5C[C@H](F)C6)nc4c3)c12]

### Synthesis of Compound 26-d

To a solution of 2,4-dichloro-7-bromoquinazoline (280 mg, 1.01 mmol) in tetrahydrofuran (15 mL) was added tert-butyl 1,2-diazepine-1-carboxylate (202 mg, 1.01 mmol) and N,N-diisopropylethylamine (390 mg, 3.02 mmol) at room temperature. The reaction mixture was stirred at room temperature overnight. Water was added to the reaction mixture and the aqueous phase was extracted with ethyl acetate (30 mL). The organic phase was dried over anhydrous sodium sulfate, filtered, and the filtrate was concentrated to dryness and the crude was purified by flash column chromatography (mobile phase: ethyl acetate/petroleum ether, 0-30%) to give compound 26-d (440 mg, 99%). LC-MS (ESI): m/z 441.1 (M+H)⁺.

### Synthesis of Compound 26-c

To a solution of 26-d (440 mg, 0.10 mmol) in toluene (10 mL) was added ((2R,7aS)-2-fluorohexahydro-1H-pyrrolizin-7a-yl)methanol (238 mg, 1.49 mmol) and sodium tert-butoxide (287 mg, 2.99 mmol) in an ice-water bath. The reaction temperature was warmed slowly to room temperature and stirred at this temperature overnight. The reaction mixture was concentrated to dryness, and the residue was added water and the aqueous phase was extracted with ethyl acetate (50 mL). The organic phase was dried over anhydrous sodium sulfate, filtered, and the filtrate was concentrated to dryness and the crude was purified by flash column chromatography (mobile phase: dichloromethane: methanol=10:1/dichloromethane, 0-100%) to give compound 26-c (538 mg, 96%). LC-MS (ESI): m/z 564.2 (M+H)⁺.

### Synthesis of Compound 26-b

To a 10 mL microwave tube were added 26-c (100 mg, 0.18 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (133 mg, 0.21 mmol), methanesulfonic acid [n-butylbis(1-adamantyl)phosphine](2-amino-1,1′-biphenyl-2-yl)palladium(II) (13 mg, 0.018 mmol), potassium phosphate (113 mg, 0.53 mmol), tetrahydrofuran (2.5 mL) and water (0.5 mL). After degassed and purged with nitrogen several times, the microwave tube was sealed. The reaction mixture was stirred at 65° C. overnight. Water was added to the reaction mixture and the aqueous phase was extracted with ethyl acetate (20 mL). The organic phase was dried over anhydrous sodium sulfate, filtered and concentrated to dryness to give compound 26-b (215 mg). The crude product was used directly for the next step without purification. LC-MS (ESI): m/z 826.9 (M+H)⁺.

### Synthesis of Compound 26-a

To a solution of 26-b (215 mg, 0.26 mmol) in N,N-dimethylformamide (10 mL) was added cesium fluoride (395 mg, 2.60 mmol) at room temperature and the mixture was stirred at room temperature overnight. Water was added to the reaction mixture and the aqueous phase was extracted with ethyl acetate (30 mL). The organic phase was washed twice with brine, dried over anhydrous sodium sulfate, filtered, and the filtrate was concentrated to dryness and the crude was purified by flash column chromatography (mobile phase: DCM: MeOH=10:1/DCM, 0-100%) to give compound 26-a (83 mg, 48%). LC-MS (ESI): m/z 670.9 (M+H)⁺.

### Synthesis of Compound 26

To a solution of 26-a (83 mg, 0.12 mmol) in acetonitrile (10 mL) was added HCl/1,4-dioxane (4 M, 0.5 mL) at room temperature and the reaction mixture was stirred at this temperature overnight. The reaction mixture was added saturated aqueous sodium bicarbonate solution to neutralize, the aqueous phase was extracted with ethyl acetate (30 mL). The organic phase was dried over anhydrous sodium sulfate, filtered, the filtrate was concentrated to dryness and the crude was purified by prep-HPLC to give compound 26 (17.1 mg, 24%). LC-MS (ESI): m/z 570.2 (M+H)⁺; ¹H NMR (400 MHz, CDCl₃): δ 8.89 (1H, dd, J=26.8, 8.8 Hz), 7.64-7.55 (2H, m), 7.32 (1H, s), 7.22-7.05 (3H, m), 7.03 (1H, d, J=7.2 Hz), 5.43-5.20 (1H,m), 4.26-3.93 (5H, m), 3.35-2.97 (6H, m), 2.78 (1H, d, J=4.4 Hz), 2.01-1.88 (6H, m), 1.78-1.62 (5H, m).

### Example 25 Synthetic Route of Compound 27

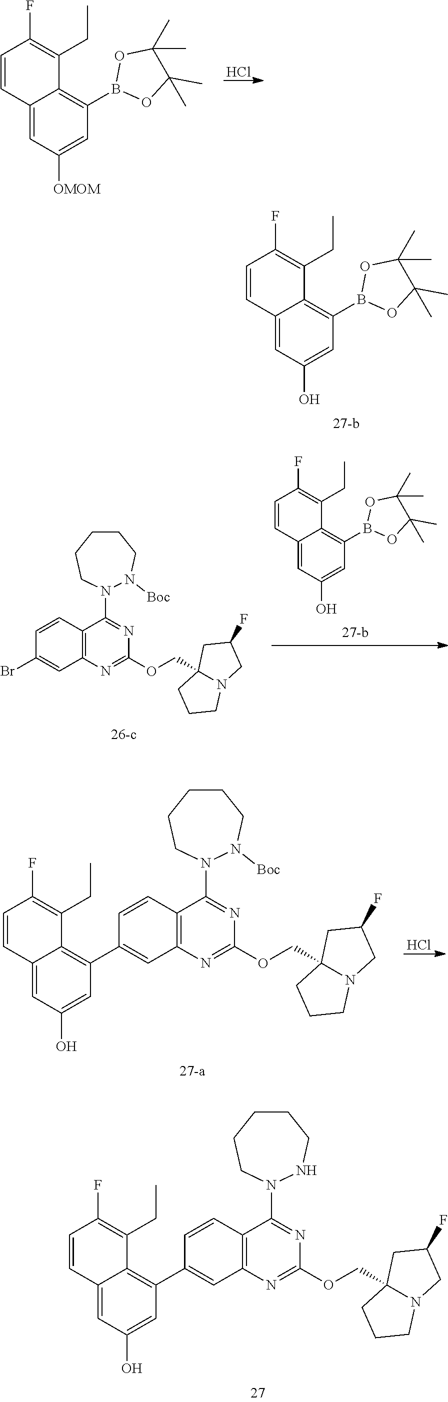

### Synthesis of Compound 27-b

To a solution of 2-(8-ethyl-7-fluoro-3-(methoxymethoxy)naphthalen-1-yl)-4,4,5,5-tetramethyl-1,3,2-dioxaborolane (500 mg, 1.39 mmol) in acetonitrile (15 mL) was added HCl/1,4-dioxane (4 M, 1.5 mL) in an ice-water bath. The mixture was stirred at this temperature for 1 h, then concentrated to dryness and the crude product was purified by flash column chromatography (mobile phase: EA/PE, 0-40%) to give compound 27-b (436 mg, 99%). LC-MS (ESI): m/z 317.0 (M+H)⁺.

### Synthesis of Compound 27-a

To a 10 mL microwave tube were added 26-c (70 mg, 0.12 mmol), 27-b (78 mg, 0.25 mmol), [n-butyl bis(1-adamantyl)phosphine](2-amino-1,1′-biphenyl-2-yl)palladium(II) methanesulfonate (9 mg, 0.012 mmol), potassium phosphate (79 mg, 0.37 mmol), tetrahydrofuran (2.5 mL) and water (0.5 mL). After degassed and purged with nitrogen several times, the microwave tube was sealed. The reaction mixture was stirred at 65° C. overnight. The reaction mixture was added water, and the aqueous phase was extracted with ethyl acetate (30 mL). The organic phase was washed twice with brine, dried over anhydrous sodium sulfate, filtered, and the filtrate was concentrated to dryness and the crude was purified by flash column chromatography (mobile phase: DCM: MeOH=10:1/DCM, 0-100%) to obtain 27-a (79 mg, 95%). LC-MS (ESI): m/z 674.9 (M+H)⁺.

### Synthesis of Compound 27

To a solution of 27-a (79 mg, 0.12 mmol) in acetonitrile (10 mL) was added HCl/1,4-dioxane (4 M, 0.5 mL) at room temperature and the reaction mixture was stirred at this temperature overnight. The reaction mixture was added saturated aqueous sodium bicarbonate solution to neutralize, extracted with ethyl acetate (30 mL). The organic phase was dried over anhydrous sodium sulfate, filtered, the filtrate was concentrated to dryness and the crude was purified by prep-HPLC to give compound 27 (26.1 mg, 39%). LC-MS (ESI): m/z 574.2 (M+H)⁺.

### Example 26 Synthetic Route of Compound 28

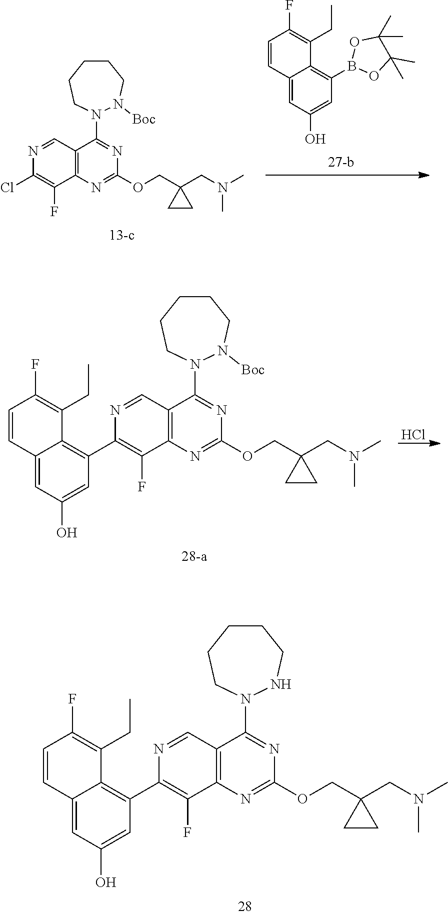 [SMILES: CN(C)CC1(COc2nc(N3CCCCCN3C(=O)OC(C)(C)C)c3cnc(Cl)c(F)c3n2)CC1 ; CCc1c(F)ccc2cc(O)cc(B3OC(C)(C)C(C)(C)O3)c12 ; CCc1c(F)ccc2cc(O)cc(-c3ncc4c(N5CCCCCN5C(=O)OC(C)(C)C)nc(OCC5(CN(C)C)CC5)nc4c3F)c12 ; Cl ; CCc1c(F)ccc2cc(O)cc(-c3ncc4c(N5CCCCCN5)nc(OCC5(CN(C)C)CC5)nc4c3F)c12]

### Synthesis of Compound 28-a

To a 10 mL microwave tube were added 13-c (50 mg, 0.098 mmol), 27-b (47 mg, 0.15 mmol), [n-butylbis(1-adamantyl)phosphine](2-amino-1,1′-biphenyl-2-yl)palladium(II) methanesulfonate (7 mg, 0.01 mmol), potassium phosphate (63 mg, 0.30 mmol), potassium tetrahydrofuran (2.5 mL) and water (0.5 mL). After degassed and purged with nitrogen several times, the microwave tube was sealed. The reaction mixture was stirred at 65° C. overnight. Water was added to the reaction mixture and the aqueous phase was extracted with ethyl acetate (30 mL). The organic phase was washed twice with brine, dried over anhydrous sodium sulfate, filtered, and the filtrate was concentrated to dryness and the crude was purified by flash column chromatography (mobile phase: DCM: MeOH=10:1/DCM, 0-100%) to give compound 28-a (56 mg, 86%). LC-MS (ESI): m/z 663.9 (M+H)⁺.

### Synthesis of Compound 28

To a solution of 28-a (56 mg, 0.084 mmol) in acetonitrile (10 mL) was added HCl/1,4-dioxane (4 M, 1 mL) at room temperature, and the reaction mixture was stirred for 3 hours at this temperature, then was neutralized by adding saturated aqueous sodium bicarbonate solution. The aqueous phase was extracted with ethyl acetate (30 mL). The organic phase was dried over anhydrous sodium sulfate, filtered, the filtrate was concentrated to dryness and the crude was purified by prep-HPLC to give compound 28 (4.2 mg, 9%). LC-MS (ESI): m/z 563.2 (M+H)⁺.

### Example 27 Synthetic Route of Compound 29

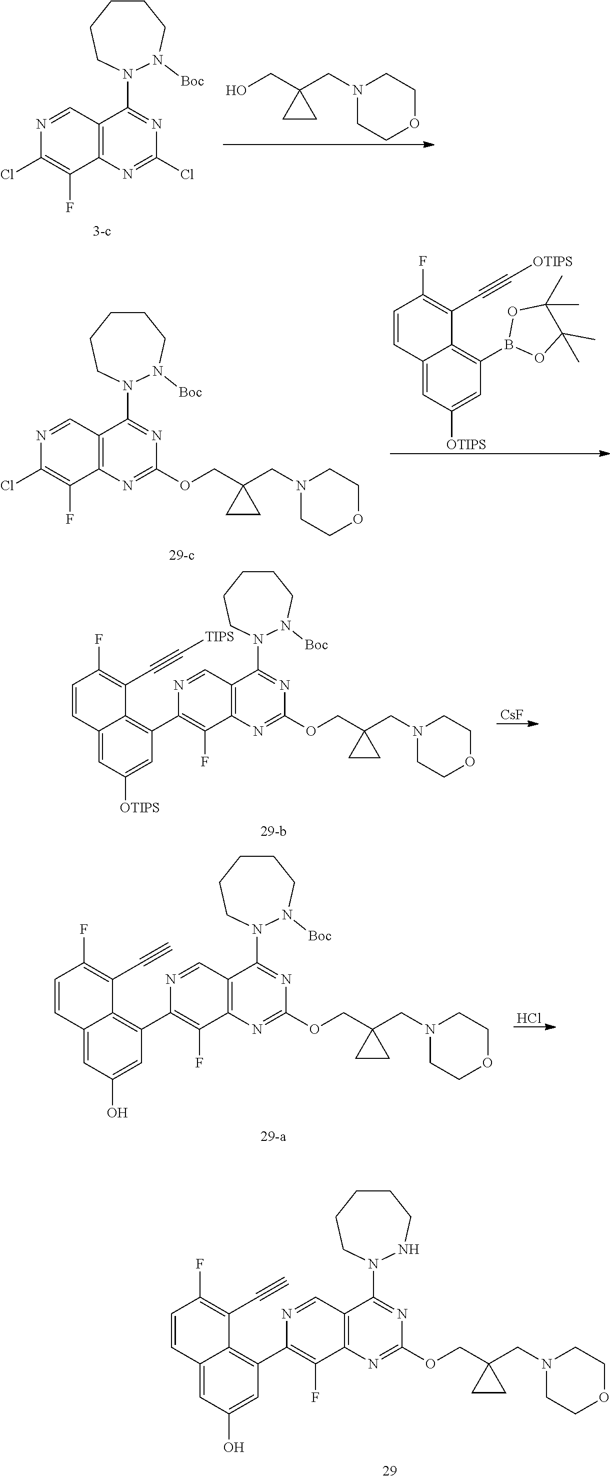

### Synthesis of Compound 29-c

To a solution of 3-c (200 mg, 0.48 mmol) in dichloromethane (20 mL) was added (1-(morpholinomethyl)cyclopropyl)methanol (165 mg, 0.96 mmol), followed by slow addition of sodium tert-butoxide (231 mg, 2.40 mmol) in an ice-water bath. The temperature was slowly warmed to room temperature and stirred at room temperature overnight. Water was added and the aqueous phase was extracted with dichloromethane (100 mL). The organic phase was dried over anhydrous sodium sulfate, filtered. The organic phase was concentrated to dryness, and the crude was purified by flash column chromatography (mobile phase: dichloromethane: methanol=10:1/dichloromethane, 0-100%) to give compound 29-c (260 mg, 98%). LC-MS (ESI): m/z 551.8 (M+H)⁺.

### Synthesis of Compound 29-b

To a 10 mL microwave tube were added 29-c (100 mg, 0.18 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (136 mg, 0.22 mmol), methanesulfonic acid [n-butylbis(1-adamantyl)phosphine](2-amino-1,1′-biphenyl-2-yl)palladium(II) (13 mg, 0.018 mmol), potassium phosphate (116 mg, 0.54 mmol), tetrahydrofuran (2.5 mL) and water (0.5 mL). After degassed and purged with nitrogen several times, the microwave tube was sealed. The reaction mixture was stirred at 65° C. overnight. Water was added to the reaction mixture and the aqueous phase was extracted with ethyl acetate (20 mL). The organic phase was dried over anhydrous sodium sulfate, filtered, and the filtrate was concentrated to dryness to obtain the crude product 29-b (180 mg, 98%). The crude product was used directly for the next step without purification. LC-MS (ESI): m/z 1013.9 (M+H)⁺.

### Synthesis of Compound 29-a

Cesium fluoride (270 mg, 1.78 mmol) was added to a solution of 29-b (180 mg, 0.18 mmol) in N,N-dimethylformamide (8 mL) at room temperature and the mixture was stirred at room temperature overnight. Water was added to the reaction mixture and the aqueous phase was extracted with ethyl acetate (30 mL). The organic phase was washed twice with brine, dried over anhydrous sodium sulfate, filtered, and the filtrate was concentrated to dryness and the crude was purified by flash column chromatography (mobile phase: MeOH/DCM, 0-100%) to give compound 29-a (40 mg, 32%). LC-MS (ESI): m/z 701.9 (M+H)⁺.

### Synthesis of Compound 29

To a solution of 29-a (40 mg, 0.057 mmol) in acetonitrile (10 mL) was added HCl/1,4-dioxane (4M, 0.5 mL) at room temperature, and the reaction mixture was stirred for 3 hours at this temperature. The reaction mixture was added saturated aqueous sodium bicarbonate solution to neutralize, extracted with ethyl acetate (30 mL). The organic phase was dried over anhydrous sodium sulfate, filtered, the filtrate was concentrated to dryness and the crude was purified by prep-HPLC to give compound 29 (1.1 mg, 3%). LC-MS (ESI): m/z 601.3 (M+H)⁺.

### Example 28 Synthetic Route of Compound 30

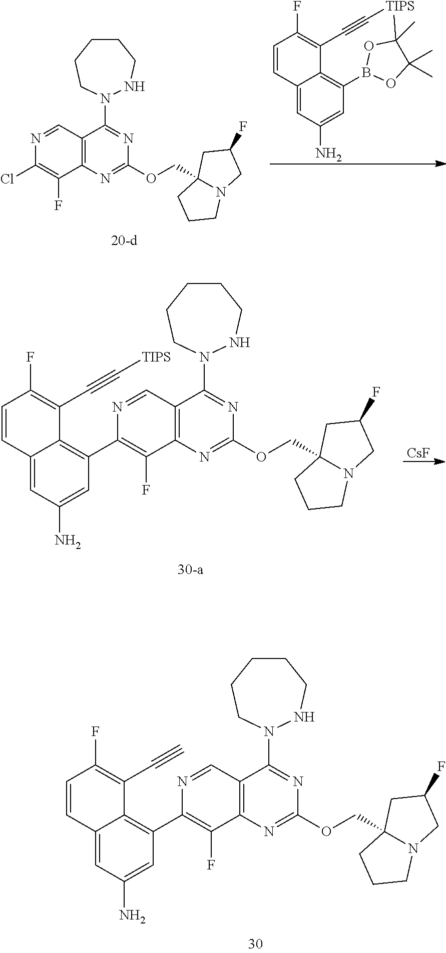

### Synthesis of Compound 30-a

To a 10 mL microwave tube were added 20-d (50 mg, 0.11 mmol), 6-fluoro-4-(4,4,5,5-tetramethyl-1,3,2-dioxaborolan-2-yl)-5-((triisopropylsilyl)ethynyl)naphthalen-2-amine (59 mg, 0.13 mmol), methanesulfonic acid [n-butylbis(1-adamantyl)phosphine] (2-amino-1,1′-biphenyl-2-yl)palladium(II) (8 mg, 0.011 mmol), potassium phosphate (73 mg, 0.34 mmol), tetrahydrofuran (2.5 mL) and water (0.5 mL). After degassed and purged with nitrogen several times, the microwave tube was sealed, and the reaction mixture was stirred at 65° C. overnight. Water was added to the reaction mixture and the aqueous phase was extracted with ethyl acetate (30 mL). The organic phase was dried over anhydrous sodium sulfate, filtered, and the filtrate was concentrated to dryness and the crude was purified by flash column chromatography (mobile phase: methanol/dichloromethane, 0-100%) to give compound 30-a (61 mg, 72%). LC-MS (ESI): m/z 744.5 (M+H)⁺.

### Synthesis of Compound 30

To a solution of 30-a (61 mg, 0.082 mmol) in N,N-dimethylformamide (8 mL) was added cesium fluoride (125 mg, 0.82 mmol) at room temperature, and the mixture was stirred at room temperature for 2 h. Water was added to the reaction mixture and the aqueous phase was extracted with ethyl acetate (30 mL). The organic phase was washed once with brine, dried over anhydrous sodium sulfate, filtered, and the filtrate was concentrated to dryness. The crude was purified by prep-HPLC to give compound 30 (17 mg, 35%). LC-MS (ESI): m/z 588.2 (M+H)⁺.

### Example 29 Synthetic Route of Compound 31

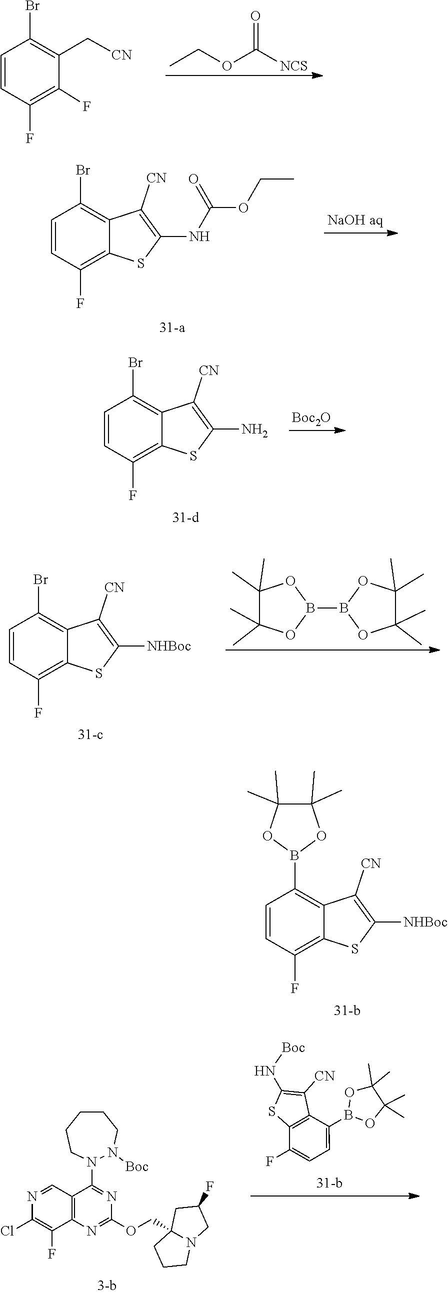

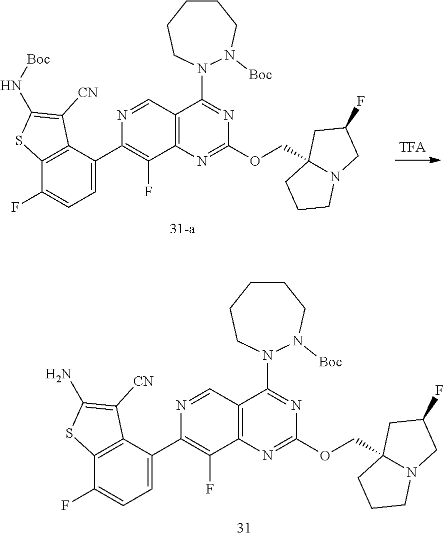 [SMILES: CC(C)(C)OC(=O)Nc1sc2c(F)ccc(-c3ncc4c(N5CCCCCN5C(=O)OC(C)(C)C)nc(OC[C@@]56CCCN5C[C@H](F)C6)nc4c3F)c2c1C#N ; CC(C)(C)OC(=O)N1CCCCCN1c1nc(OC[C@@]23CCCN2C[C@H](F)C3)nc2c(F)c(-c3ccc(F)c4sc(N)c(C#N)c34)ncc12]

### Synthesis of Compound 31-e

Potassium tert-butoxide (203 mg, 1.81 mmol) was added to a solution of 2-(6-bromo-2,3-difluorophenyl)acetonitrile (400 mg, 1.72 mmol) in N,N-dimethylformamide (10 mL) under the protection of nitrogen, and the resulting mixture was stirred at room temperature for 30 min, and then was added ethoxycarbonyl isothiocyanate (237 mg, 1.81 mmol) dropwise. After the addition, the reaction mixture was continued stirring at room temperature for 1 hour, and then stirred at 100° C. for 1 hour. The reaction mixture was poured into 50 mL of water and stirred for 10 min, the solid was collected by filtration and dried to obtain 31-e (550 mg, 93%). LC-MS (ESI): m/z 340.8(M−H); ¹H NMR (400 MHz, DMSO-d₆): δ 12.10 (1H, s), 7.68 (1H, dd, J=8.8, 4.8 Hz), 7.21 (1H, t, J=8.8 Hz), 4.30 (2H, q, J=7.2 Hz), 1.31 (3H, t, J=7.2 Hz).

### Synthesis of Compound 31-d

31-e (300 mg, 0.87 mmol) was dissolved in dimethyl sulfoxide (9 mL), followed by addition of 5 M aqueous sodium hydroxide (9 mL, 45 mmol). The reaction mixture was stirred at 130° C. for 2 hours. The reaction mixture was poured into water and solids were precipitated, stirred for 30 min at room temperature and held still overnight. The next day, it was filtered, the solid was collected and dried to give compound 31-d (220 mg, 93%). LC-MS (ESI): m/z 268.9 (M−H)⁻.

### Synthesis of Compound 31-c

To a solution of 31-d (220 mg, 0.81 mmol) in tetrahydrofuran (20 mL) was added di-tert-butyl dicarbonate (531 mg, 2.43 mmol), N,N-diisopropylethylamine (524 mg, 4.06 mmol) and 4-dimethylaminopyridine (10 mg, 0.081 mmol) and the mixture was stirred at room temperature overnight. Water was added to the reaction mixture and the aqueous phase was extracted with ethyl acetate (50 mL). The organic phase was dried over anhydrous sodium sulfate, filtered, and the filtrate was concentrated to dryness and the crude was purified by column chromatography (mobile phase: EA/PE, 0-20%) to give compound 31-c (232 mg, 77%). LC-MS (ESI): m/z 369.0(M−H)⁻.

### Synthesis of Compound 31-b

To a 30 mL microwave tube were added 31-c (50 mg, 0.13 mmol), bis-pinacol borate (171 mg, 0.67 mmol), [1,1-bis(diphenylphosphino)ferrocene]palladium dichloride (10 mg, 0.013 mmol), potassium acetate (66 mg, 0.673 mmol) and 1,4-dioxane (8 mL). After degassed and purged with nitrogen several times, the microwave tube was sealed, and the reaction mixture was stirred at 100° C. under microwave conditions for 2 h. Water was added to the reaction mixture and the aqueous phase was extracted with ethyl acetate (30 mL). The organic phase was dried over anhydrous sodium sulfate, filtered, and the filtrate was concentrated to dryness and the crude was purified by column chromatography (mobile phase: EA/PE, 0-20%) to give compound 31-b (50 mg, 89%). LC-MS (ESI): m/z 417.1(M−H)⁻.

### Synthesis of Compound 31-a

3-b (100 mg, 0.19 mmol), 31-b (86 mg, 0.21 mmol), dichloro[bis(diphenylphosphinophenyl)ether]palladium(II) (13 mg, 0.019 mmol), cesium carbonate (121 mg, 0.37 mmol), and toluene (4 mL) were added to a 10 mL microwave tube at room temperature. After degassed and purged with nitrogen several times, the microwave tube was sealed. The reaction mixture was stirred at 100° C. and stirred for 2 hours. The reaction mixture was concentrated to dryness, water was added and the aqueous phase was extracted with ethyl acetate (30 mL). The organic phase was dried over anhydrous sodium sulfate, filtered, and the filtrate was concentrated to dryness and the crude was purified by flash column chromatography (mobile phase: dichloromethane: methanol=10:1/dichloromethane, 0-100%) to give compound 31-a (75 mg, 51%).

LC-MS (ESI): m/z 795.9 (M+H)⁺.

### Synthesis of Compound 31

Trifluoroacetic acid (2 mL) was added to a solution of 31-a (75 mg, 0.094 mmol) in dichloromethane (8 mL) at room temperature and the reaction mixture was stirred for 2 h at this temperature. The reaction mixture was neutralized by adding saturated aqueous sodium bicarbonate solution to the reaction mixture and the aqueous phase was extracted with dichloromethane (30 mL). The organic phase was dried over anhydrous sodium sulfate, filtered, and the filtrate was concentrated to dryness and the crude was purified by reversed-phase over column (mobile phase: 5%-⁹⁵%, water (10 mmol ammonium bicarbonate): acetonitrile) and Prep-TLC (eluent: dichloromethane: methanol=10:1) to give compound 31 (15 mg, 27%). LC-MS (ESI): m/z 595.2 (M+H)⁺.

### Example 30 Synthetic Route of Compound 32

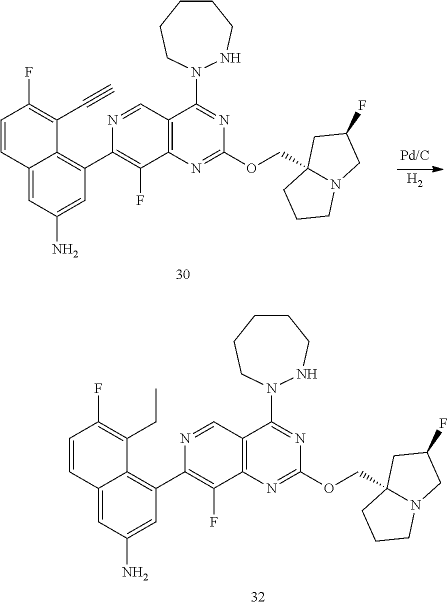

### Synthesis of Compound 32

Compound 30 (60 mg, 0.102 mmol) was dissolved in ethyl acetate (15 mL) and methanol (2 mL), followed by addition of Pd/C 10% (100 mg). After degassed and purged with hydrogen several times, the reaction mixture was stirred for 4 hours at room temperature. The Pd/C was removed by filtration.

The organic phase was concentrated to dryness and the crude was purified by prep-HPLC to give compound 32 (5.5 mg, 9%). LC-MS (ESI): m/z 592.2 (M+H)⁺.

### Example 31 Synthetic Route of Compound 33

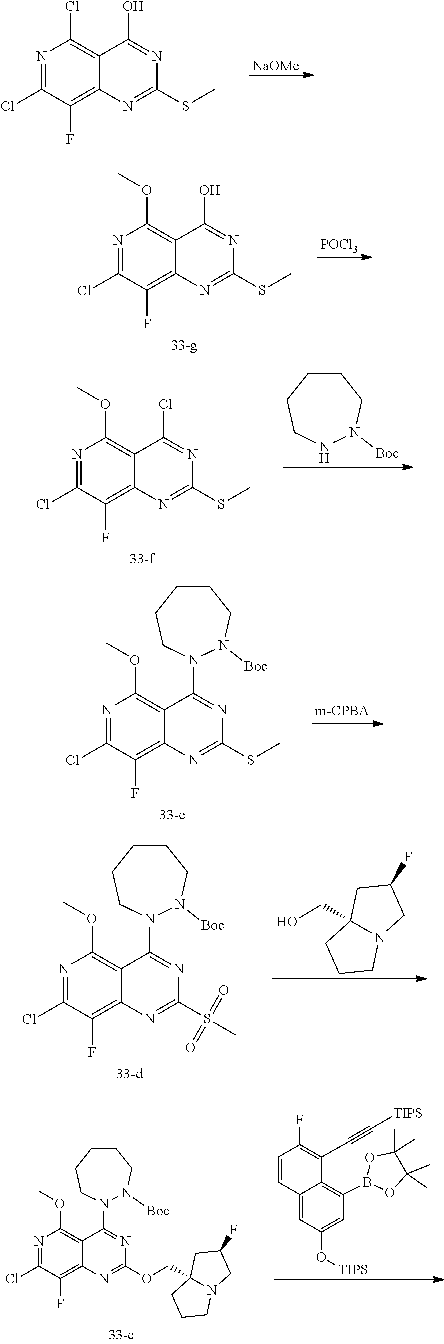

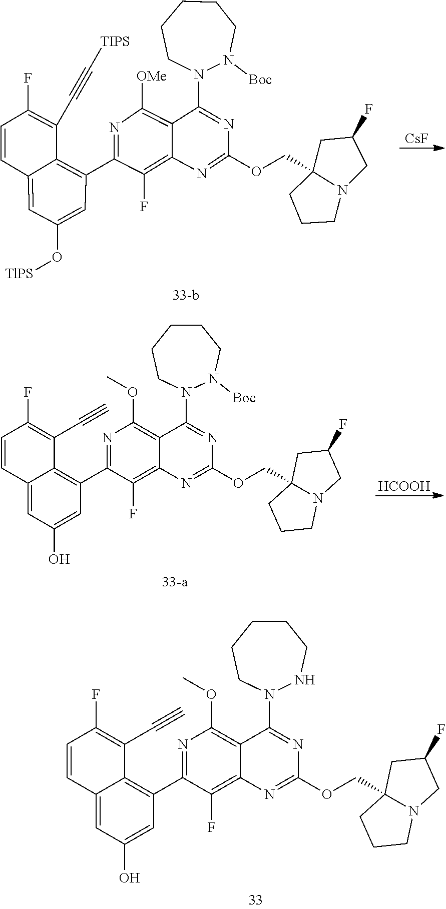

### Synthesis of Compounds 33-g

At room temperature, compound 5,7-dichloro-8-fluoro-2-(methylthio)pyrido[4,3-d]pyrimidin-4(3H)-one (1.0 g, 3.57 mmol) was dissolved in anhydrous DMAc (10 mL), to which anhydrous methanol (2 mL), sodium methanol (386 mg, 7.14 mmol) were added sequentially. The reaction mixture was stirred under protection of nitrogen at 50° C. for 2 hours. The reaction mixture was cooled in an ice-water bath and the pH was adjusted to 3 with 1 M hydrochloric acid solution, then 20 mL of water was added and a large amount of solid precipitated. The mixture was filtered and the filter cake was washed with water (10 mL*3), the solid was collected and lyophilized overnight to give compound 33-g (930 mg, 95%). LC-MS (ESI): m/z=276.1 [M+H]⁺.

### Synthesis of Compound 33-f

Compound 33-g (930 mg, 3.37 mmol) was dissolved in 20 mL of anhydrous acetonitrile at room temperature, DIPEA (4.18 mL, 25.30 mmol), and phosphorus trichloride (4.15 mL, 44.53 mmol) were added to the above mixture. The reaction mixture was stirred at 80° C. under protection of nitrogen for 1 hour. The reaction mixture was cooled to room temperature, concentrated at reduced pressure and the residue was purified by flash column chromatography (EA/PE=0-30%) to give compound 33-f (775 mg, 78%). LC-MS (ESI): m/z=293.9[M+H]⁺.

### Synthesis of Compound 33-e

Compound 33-f (775 mg, 2.63 mmol) was dissolved in 50 mL of dichloromethane at room temperature, to which DIPEA (1.31 mL, 7.91 mmol) and tert-butyl 1,2-diazepine-1-carboxylate (528 mg, 2.64 mmol) were added in an ice-water bath. The reaction mixture was warmed to room temperature and stirred for 1 hour. The reaction mixture was added 50 mL of water, and the aqueous phase was extracted with dichloromethane (100 mL*2). The organic phase was washed sequentially with 10% sodiumbisulfate solution (100 mL*2), saturated sodium bicarbonate (100 mL), dried over anhydrous sodium sulfate, filtered, the filtrate was concentrated at reduced pressure, and the residue was purified by a flash column chromatography (PE/EA=2:1) to afford compound 33-e (980 mg, 81%). LC-MS (ESI): m/z=458.2[M+H]⁺.

### Synthesis of Compound 33-d

Compound 33-e (980 mg, 2.14 mmol) was dissolved in 30 mL of ethyl acetate at room temperature and m-chloroperoxybenzoic acid (1.13 g, 5.56 mmol) was added. The reaction mixture was stirred at room temperature under protection of nitrogen for 1 hour. The reaction mixture was added 100 mL of saturated sodium bicarbonate, and the aqueous phase was extracted with ethyl acetate (150 mL). The organic phase was washed with saturated sodium bicarbonate (100 mL) and brine (100 mL) sequentially, dried over anhydrous sodium sulfate, filtered, the filtrate was concentrated at reduced pressure, and the residue was purified by a flash column chromatography (PE/EA=2:1) to afford compound 33-d (479 mg, 46%). LC-MS (ESI): m/z=490.2 [M+H]⁺.

### Synthesis of Compound 33-c

Compound 33-d (200 mg, 0.41 mmol) was dissolved in 20 mL of dichloromethane at room temperature, ((2R,7aS)-2-fluorohexahydro-1H-pyrrolizin-7a-yl)methanol (78 mg, 0.49 mmol) was added, followed by addition of sodium tert-butanolate (98 mg, 1.02 mmol) in an ice-water bath and the reaction mixture was stirred in an ice-water bath for 2 hour. The reaction mixture was added 50 mL of water, and the aqueous phase was extracted with dichloromethane (100 mL). The organic phase was dried over anhydrous sodium sulfate, filtered, and the filtrate was concentrated at reduced pressure, and the residue was purified by a flash column chromatography (EA/PE=0-100%) to give compound 33-c (128 mg, 55%). LC-MS (ESI): m/z=569.5 [M+H]⁺.

### Synthesis of Compound 33-b

Compound 33-c (128 mg, 0.22 mmol), ((6-fluoro-4-(4,4,5,5-tetramethyl-1,3,2-dioxaborolan-2-yl)-5-((triisopropylsilyl)ethynyl)naphthalen-2-yl)oxy)triisopropylsilane(211 mg, 0.34 mmol), methanesulfonyloxy(diadamantyl-n-butylphosphino)-2′-amino-1,1′-biphenyl-2-yl)palladium(II) (33 mg, 0.045 mmol), potassium phosphate (143 mg, 0.68 mmol), THF (2.5 mL), and water (0.5 mL) were combined in a microwave tube. The mixture was degassed and purged with nitrogen for three times, sealed, and stirred at 65° C. for 18 hours. The mixture was cooled to room temperature, 50 mL of water was added, and the aqueous phase was extracted with ethyl acetate (100 mL). The organic phase was dried over anhydrous sodium sulfate, filtered, and the filtrate was concentrated at reduced pressure, and the residue was purified by a flash column chromatography (PE/EA, then DCM/MeOH=10:1) to give compound 33-b (53 mg, 23%). LC-MS (ESI): m/z=1031.4 [M+H]⁺.

### Synthesis of Compound 33-a

Compound 33-b (53 mg, 0.051 mmol) was dissolved in 5 mL of DMF at room temperature. Cesium fluoride (156 mg, 1.03 mmol) was added, and the mixture was stirred under protection of nitrogen for 8 h at room temperature. The reaction mixture was added 50 mL of water, and the aqueous phase was extracted with ethyl acetate (50 mL*2). The organic phase was washed with brine (100 mL*3), dried over anhydrous sodium sulfate, filtered, and the filtrate was concentrated at reduced pressure, and the residue was purified by a flash column chromatography (DCM/MeOH=10:1) to give compound 33-a (30 mg, 82%). LC-MS (ESI): m/z=719.0 [M+H]⁺.

### Synthesis of Compound 33

Compound 33-a (30 mg, 0.042 mmol) was dissolved in 5 mL of formic acid at room temperature and the mixture was stirred under protection of nitrogen for 3 hours at room temperature. The reaction mixture was concentrated at reduced pressure and 20 mL of dichloromethane was added to the residue, which was concentrated at reduced pressure to give compound the crude product, which was purified by prep-HPLC (NH₄HCO₃) to give compound 33 (8.9 mg, 34%). LC-MS(ESI):nm/z=619.3[MS+H]⁺.

### Example 32 Synthetic Route of Compound 34

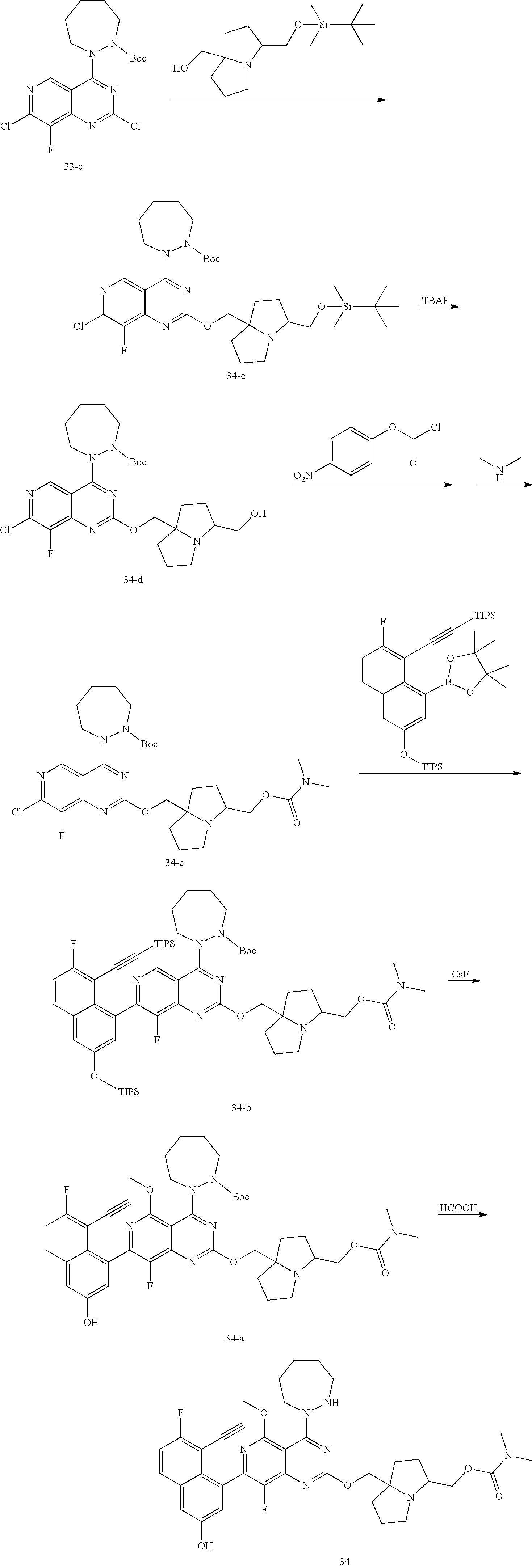

### Synthesis of Compound 34-e

To a solution of compound 3-c (500 mg, 1.20 mmol) in 30 mL of dichloromethane at room temperature was added [3-[[tert-butyl(dimethyl)silyl]oxymethyl]-1,2,3,5,6,7-hexahydropyrrolozin-8-yl]methanol (377 mg, 1.32 mmol), followed by addition of sodium tert-butanol (289 mg, 3.00 mmol) in an ice-water bath, and the reaction mixture was stirred for 3 hours in an ice-water bath. The reaction mixture was added 50 mL of water, and the aqueous phase was extracted with dichloromethane (100 mL). The organic phase was dried over anhydrous sodium sulfate, filtered, and the filtrate was concentrated at reduced pressure, and the residue was purified by a flash column chromatography (DCM/MeOH=10:1) to give compound 34-e (700 mg, 88%).

### Synthesis of Compound 34-d

A solution of tetrabutylammonium fluoride in tetrahydrofuran (2.10 mL, 2.10 mmol, 1 M) was added into a solution of compound 34-e (700 mg, 1.05 mmol) in 20 mL of anhydrous tetrahydrofuran in an ice-water bath, and the reaction mixture was stirred for 2.5 h at room temperature. The reaction mixture was concentrated at reduced pressure, the residue was dissolved in 100 mL of ethyl acetate, washed sequentially with water (100 mL), brine (100 mL). The organic phase was dried over anhydrous sodium sulfate, filtered, the filtrate was concentrated at reduced pressure, and the residue was dried under vacuum overnight to obtain compound 34-d (579 mg, 100%). LC-MS (ESI): m/z=551.1 [M+H]⁺.

### Synthesis of Compound 34-c

Compound 34-d (460 mg, 0.83 mmol) was dissolved in 30 mL of anhydrous tetrahydrofuran at room temperature, followed by addition of p-nitrophenyl chloroformate (505 mg, 2.50 mmol), triethylamine (406 uL, 2.92 mmol) sequentially, and the reaction mixture was stirred for 1 h at room temperature. P-nitrophenyl chloroformate (505 mg, 2.50 mmol) and triethylamine (406 uL, 2.92 mmol) were added and the mixture was stirred at room temperature for 1 hour. Again p-nitrophenyl chloroformate (505 mg, 2.50 mmol) and triethylamine (406 uL, 2.92 mmol) were added and the reaction mixture was stirred at room temperature for 1 hour. A solution of dimethylamine in tetrahydrofuran (8.35 mL, 16.70 mmol, 2M) was added to the reaction mixture and stirred for 18 hours. A solution of dimethylamine in tetrahydrofuran (5 mL) in tetrahydrofuran was then added every 1 hour until the completion of the reaction, with a total of three additions of a solution of dimethylamine in tetrahydrofuran. To the reaction mixture 100 mL of water was added, the aqueous phase was extracted with ethyl acetate (100 mL). The organic phase was washed with brine (100 mL), dried over anhydrous sodium sulfate, filtered, the filtrate was concentrated at reduced pressure, and the residue was purified by a flash column chromatography (DCM/MeOH=30:1-10 :1) to give compound 34-c (240 mg, 47%). LC-MS (ESI): m/z=622.3 [M+H]⁺.

### Synthesis of Compound 34-b

Compound 34-c (240 mg, 0.39 mmol), ((6-fluoro-4-(4,4,5,5-tetramethyl-1,3,2-dioxaborolan-2-yl)-5-((triisopropylsilyl)ethynyl)naphthalen-2-yl)oxy)triisopropylsilane (362 mg, 0.58 mmol), methylsulfonyloxy(di adamantyl-n-butylphosphino)-2′-amino-1,1′-biphenyl-2-yl)palladium(II) (56 mg, 0.077 mmol), potassium phosphate (246 mg, 1.16 mmol), THF (5 mL), and water (1 mL) were combined in a microwave tube. The mixture was degassed and purged with nitrogen for three times, sealed, and stirred for 18 hours at 65° C. The mixture was cooled to room temperature, was added 100 mL of water and the aqueous phase was extracted with ethyl acetate (100 mL). The organic phase was dried over anhydrous sodium sulfate, filtered, and the filtrate was concentrated at reduced pressure, and the residue was purified by a flash column chromatography (DCM/MeOH=10:1) to give the crude compound 34-b (418 mg, as a mixture). LC-MS (ESI): m/z=928.4 [M+H, de-TIPS]+; LC-MS (ESI): m/z=1084.5 [M+H]⁺.

### Synthesis of Compound 34-a

Compound 34-b (418 mg, 0.39 mmol) was dissolved in 30 mL of DMF at room temperature, followed by addition of cesium fluoride (1.17 g, 7.71 mmol), and the mixture was stirred at room temperature under protection of nitrogen for 3.5 hours. The reaction mixture was added 100 mL of water, and the aqueous phase was extracted with ethyl acetate (100 mL). The organic phase was washed with brine (100 mL*5), dried over anhydrous sodium sulfate, filtered, and the filtrate was concentrated at reduced pressure, and the residue was purified by a flash column chromatography (DCM/MeOH=10:1) to give compound 34-a (266 mg, 88%). LC-MS (ESI). m/z=772.4 [M+H]⁺.

### Synthesis of Compound 34

Compound 34-a (100 mg, 0.13 mmol) was dissolved in 15 mL of formic acid at room temperature and the mixture was stirred at room temperature under protection of nitrogen for 18 hours. The reaction mixture was concentrated at reduced pressure, 50 mL of saturated sodium bicarbonate was added to the residue and the aqueous phase was extracted with dichloromethane (50 mL*2). The organic phase was dried over anhydrous sodium sulfate, filtered, and the filtrate was concentrated at reduced pressure to obtain the crude product, which was purified by a flash column chromatography (DCM/MeOH=10:1) to give compound 34 (25 mg, 29%). LC-MS (ESI): m/z=672.2[MS+H]⁺.

### Example 33 Synthetic Route of Compound 35

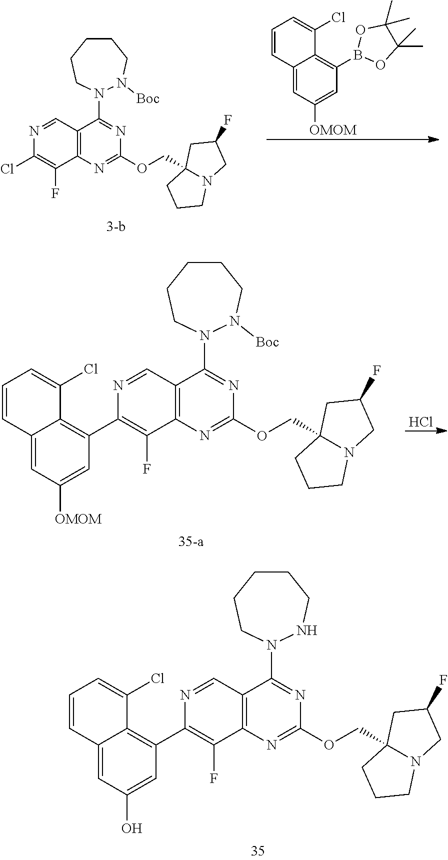

### Synthesis of Compound 35-a

To a sealed tube at room temperature was added 3-b (150 mg, 0.28 mmol), 2-(8-chloro-3-(methoxymethoxy)naphthalen-1-yl)-4,4,5,5-tetramethyl-1,3,2-dioxaborolane (146 mg, 0.42 mmol), cataCXium A Pd G3 (20 mg, 0.03 mmol), potassium phosphate (177 mg, 0.83 mmol), THF (2 mL) and water (0.5 mL). After degassed and purged with nitrogen, the reaction mixture was stirred at 65° C. overnight. The reaction mixture was concentrated and the residue was purified by column chromatography (mobile phase: DCM/MeOH 10/0 to 10/1) to afford compound 35-a (160 mg, 79%).

LC-MS (ESI): m/z 725.8 (M+H)⁺.

### Synthesis of Compound 35

At room temperature, to a reaction vial were added 35-a (130 mg, 0.18 mmol), acetonitrile (5 mL). After degassed and purged with nitrogen, the reaction mixture was cooled to 0° C. and was added HCl/1,4-dioxane (4.0 M, 1 mL), then stirred at room temperature for 3 hours. The reaction mixture was concentrated at room temperature and the residue was purified by prep-HPLC to give compound 35 (16 mg, 15%). LC-MS (ESI): m/z 581.7 (M+H)⁺.

### Example 34 Synthetic Route of Compound 36

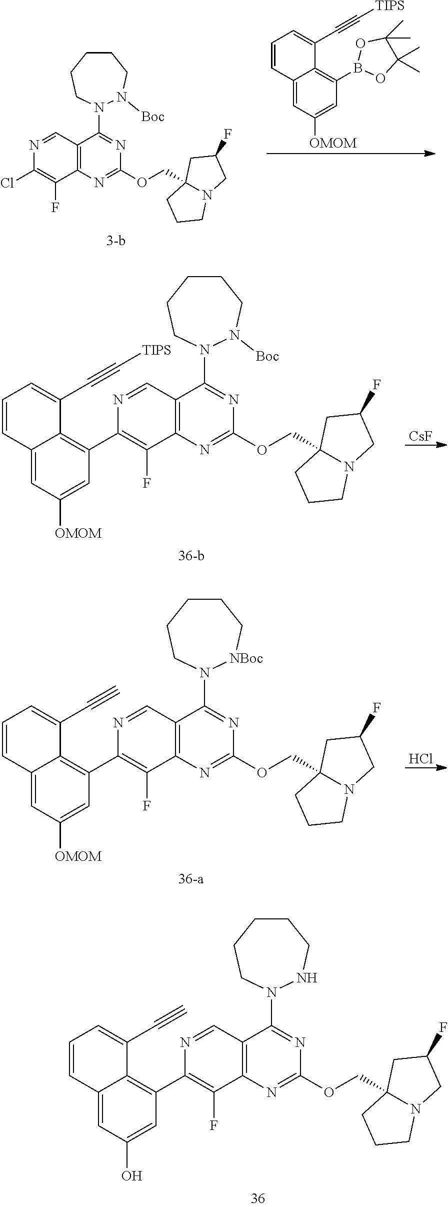

### Synthesis of Compound 36-b

To the sealed tube at room temperature were added 3-b (200 mg, 0.37 mmol), triisopropyl((6-(methoxymethoxy)-8-(4,4,5,5-tetramethyl-1,3,2-dioxaborolan-2-yl)naphthalen-1-yl)ethynyl)silane (275 mg, 0.55 mmol), cataCXium A Pd G3 (54 mg, 0.07 mmol), potassium phosphate (236 mg, 1.11 mmol), THF (2 mL) and water (0.5 mL). After degassed and purged with nitrogen, the reaction mixture was stirred at 65° C. overnight. The reaction mixture was concentrated and the residue was purified by column chromatography (mobile phase: DCM/MeOH 10/0 to 10/1) to afford compound 36-b (300 mg, 93%). LC-MS (ESI): m/z 871.9 (M+H)⁺.

### Synthesis of Compound 36-a

36-b (140 mg, 0.16 mmol), DMF (2 mL), and cesium fluoride (244 mg, 1.61 mmol) were added to a reaction flask at room temperature. After degassed and purged with nitrogen, the reaction mixture was stirred at 60° C. for 1 hr. The reaction mixture was added with water, and the aqueous phase was extracted with ethyl acetate. The organic phases were combined, washed with water, dried over anhydrous sodium sulfate, and concentrated to give the crude compound 36-a (115 mg). LC-MS (ESI): m/z 715.8 (M+H)⁺.

### Synthesis of Compound 36

At room temperature, to a reaction flask were added crude compound 36-a (115 mg, 0.16 mmol), acetonitrile (5 mL). After degassed and purged with nitrogen, the above mixture was cooled down to 0° C., and was added HCl/1,4-dioxane (4.0 M, 1 mL), then stirred at room temperature for 3 hours. The reaction mixture was neutralized with aqueous sodium bicarbonate and extracted by DCM. The organic phases were combined, washed with water, dried over anhydrous sodium sulfate, concentrated and the residue was purified by prep-HPLC to give compound 36 (11 mg, 12%). LC-MS (ESI): m/z 571.6 (M+H)⁺; ¹H NMR (400 MHz, DMSO-d₆): δ 10.24 (1H, s), 10.12 (1H, m), 7.92-7.81 (1H, d, J=8.0 Hz ), 7.49-7.36 (2H, m), 7.36-7.26 (1H, d, J=2.4 Hz ), 7.14-7.04 (1H, d, J=2.0 Hz ), 5.92-5.78 (1H, t, J=6.0 Hz ), 5.40-5.13 (1H, d, J=54.0 Hz), 4.18-3.88 (4H, m), 3.56 (1H, s), 3.17-2.95 (5H, m), 2.90-2.75 (1H, m), 2.19-1.54 (12H, m).

### Example 35 Synthetic Route of Compound 37

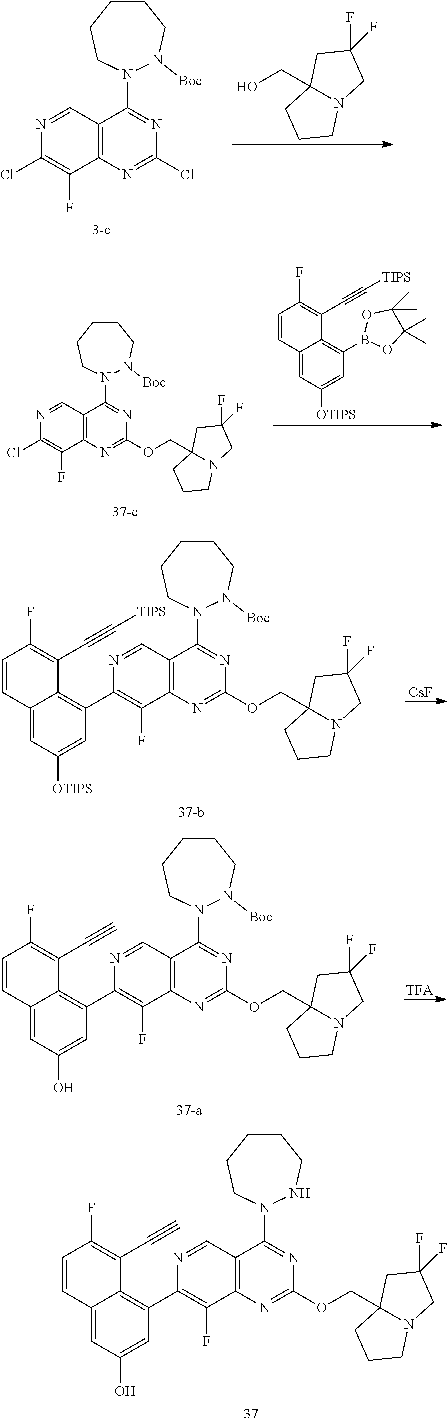

### Synthesis of Compound 37-c

At room temperature, to a reaction vial were added 3-c (400 mg, 0.96 mmol), (2,2-difluorohexahydro-1H-pyrrolizin-7a-yl)methanol (255 mg, 1.44 mmol), 1,4-dioxane (10 mL), DIPEA (373 mg, 2.89 mmol). After degassed and purged with nitrogen, the reaction mixture was stirred at 90° C. overnight. The reaction mixture was concentrated and the residue was purified by column chromatography (mobile phase: DCM/MeOH 10/0 to 10/1) to give compound 37-c (300 mg, 56%). LC-MS (ESI): m/z 557.7 (M+H)⁺.

### Synthesis of Compound 37-b

37-c (300 mg, 0.54 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (505 mg, 0.81 mmol) were added to a sealed tube at room temperature, followed by combination of cataCXium A Pd G3 (78 mg, 0.11 mmol), potassium phosphate (343 mg, 1.62 mmol), THF (2 mL) and water (0.5 mL) in the sealed tube. After degassed and purged with nitrogen, the reaction mixture was stirred at 65° C. overnight. The reaction mixture was concentrated and the residue was purified by column chromatography (mobile phase: DCM/MeOH 10/0 to 10/1) to give compound 37-b (460 mg, 84%).

### Synthesis of Compound 37-a

At room temperature, to a reaction vial were added 37-b (460 mg, 0.45 mmol), DMF (2 mL), and cesium fluoride (684 mg, 4.50 mmol). After degassed and purged with nitrogen, the reaction mixture was stirred for 2 hours at room temperature. The reaction mixture was added water and the aqueous phase was extracted by EA. The organic phases were combined, washed with water, dried over anhydrous sodium sulfate and concentrated to give the crude compound 37-a (500 mg). LC-MS (ESI): m/z 707.7 (M+H)⁺.

### Synthesis of Compound 37

At room temperature, to a reaction vial were added crude compound 37-a (500 mg, 0.45 mmol), DCM (10 mL) and TFA (3 mL). After stirred at room temperature for 2 hours, the reaction mixture was neutralized with aqueous sodium bicarbonate and the aqueous phase was extracted with DCM:MeOH=10:1 solution. The organic phases were combined, washed with water, dried over anhydrous sodium sulfate, concentrated and the residue was purified by prep-HPLC to give compound 37 (4.5 mg, 2%). LC-MS (ESI): m/z 607.5 (M+H)⁺.

### Example 36 Synthetic Route of Compound 38

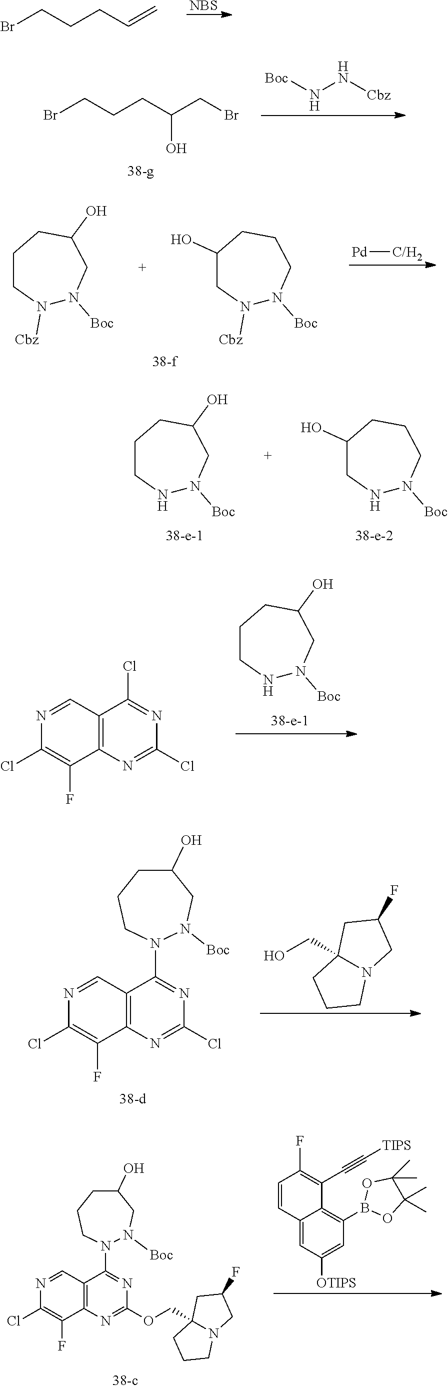

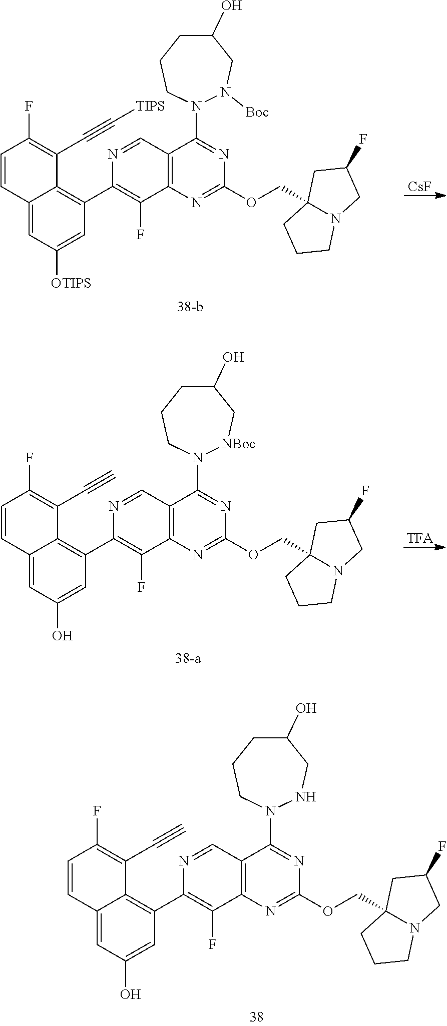

### Synthesis of Compound 38-g

NBS (2150 mg, 12.08 mmol), acetonitrile (28 mL), water (7 mL) and 5-bromo-1-pentene (1500 mg, 10.07 mmol) were added to a reaction vial at room temperature. The reaction mixture was stirred at room temperature for 36 hours. 90 mL of water was added to the reaction mixture and the aqueous phase was extracted with DCM three times. The organic phases were combined, washed with water, brine, dried over anhydrous sodium sulfate, concentrated, and the residue was purified by column chromatography (mobile phase: PE/EA 10/0 to 10/2) to give compound 38-g (800 mg, 32%).

### Synthesis of Compound 38-f

At room temperature, to a reaction vial were added 2-(tert-butyl)hydrazine-1,2-dicarboxylic acid-1-benzyl ester (780 mg, 2.93 mmol) and DMF (25 mL). After degassed and purged with nitrogen, the above mixture was added NaH (60%, 234 mg, 5.85 mmol) in batches in an ice-water bath and warmed to room temperature with stirring for 1 hour. A solution of 38-g (800 mg, 3.25 mmol) in DMF (10 mL) was added dropwise in an ice-water bath. After addition, the reaction mixture was warmed to room temperature overnight. The reaction mixture was quenched with saturated aqueous ammonium chloride and the aqueous phase was extracted with ethyl acetate. The organic phases were combined, washed with water, dried, concentrated, and the residue was purified by column chromatography (mobile phase: PE/EA 10/0 to 10/2) to give compound 38-f (500 mg, 49%). LC-MS (ESI): m/z 351.2 (M+H)⁺.

### Synthesis of Compounds 38-e-1, 38-e-2

38-f (1000 mg, 2.85 mmol), EA (100 mL) and Pd/C (10%, 200 mg) were added to a reaction vial at room temperature. The reaction mixture was degassed and purged with hydrogen, then stirred under atmosphere of hydrogen for 2 hours at room temperature. The reaction mixture was filtered, and the filtrate was concentrated and the residue was purified by column chromatography (mobile phase: PE/EA 10/0 to 1/1) to give compounds 38-e-1 (350 mg, 57%) and 38-e-2 (180 mg, 29%). LC-MS (ESI): m/z 217.1 (M+H)⁺.

### Synthesis of Compound 38-d

2,4,7-Trichloro-8-fluoropyrido[4,3-d]pyrimidine (2066 mg, 8.18 mmol) and DCM (30 mL) were added to a reaction vial at room temperature. After degassed and purged with nitrogen, the above mixture was cooled to −78° C. in a dry ice-acetone bath, then was added DIPEA (2116 mg, 16.37 mmol) and 38-e-1 (590 mg, 2.73 mmol). After addition, the mixture was naturally warmed to room temperature and stirred overnight. Water was added to the reaction mixture, and the aqueous phase was extracted with DCM. The organic phases were combined, dried over anhydrous sodium sulfate, concentrated, and the residue was purified by column chromatography (mobile phase: PE/EA 10/0 to 7/3) to give compound 38-d (620 mg, 53%).

### Synthesis of Compound 38-c

At room temperature, to a reaction vial were added 38-d (300 mg, 0.69 mmol), ((2R,7aS)-2-fluorohexahydro-1H-pyrrolizin-7a-yl)methanol (221 mg, 1.39 mmol), 1,4-dioxane (10 mL) and DIPEA (269 mg, 2.08 mmol). After degassed and purged with nitrogen, the reaction mixture was stirred at 90° C. overnight. The reaction mixture was concentrated and the residue was purified by column chromatography (mobile phase: DCM/MeOH 10/0 to 10/1) to give compound 38-c (220 mg, 57%). LC-MS (ESI): m/z 555.9 (M+H)⁺.

### Synthesis of Compound 38-b

To a sealed tube at room temperature were added 38-c (220 mg, 0.40 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (372 mg, 0.60 mmol), cataCXium A Pd G3 (58 mg, 0.08 mmol), potassium phosphate (252 mg, 1.19 mmol), THF (2 mL), and water (0.5 mL). After degassed and purged with nitrogen, the reaction mixture was stirred at 65° C. overnight. The reaction mixture was concentrated and the residue was purified by column chromatography (mobile phase: DCM/MeOH 10/0 to 10/1) to give compound 38-b (230 mg, 57%). LC-MS (ESI): m/z 1018.0 (M+H)⁺.

### Synthesis of Compound 38-a

38-b (230 mg, 0.23 mmol), DMF (2 mL), and cesium fluoride (343 mg, 2.26 mmol) were added to a reaction vial at room temperature. After degassed and purged with nitrogen, the reaction mixture was stirred for 2 hours at room temperature. The reaction mixture was added water and the aqueous phase was extracted by EA. The organic phases were combined, washed with water, dried over anhydrous sodium sulfate and concentrated to give the crude compound 38-a (300 mg). LC-MS (ESI): m/z 705.9 (M+H)⁺.

### Synthesis of Compound 38

At room temperature, to a reaction vial were added crude compound 38-a (300 mg, 0.23 mmol), DCM (6 mL) and TFA (2 mL). After stirred at room temperature for 1.5 hours, the reaction mixture was neutralized with aqueous sodium bicarbonate and the aqueous phase was extracted with DCM:MeOH=10:1 solution. The organic phases were combined, washed with water, dried over anhydrous sodium sulfate, concentrated and the residue was purified by prep-HPLC to give compound 38 (27 mg, 20%). LC-MS (ESI): m/z 605.5 (M+H)⁺.

### Example 37 Synthetic Route of Compound 39

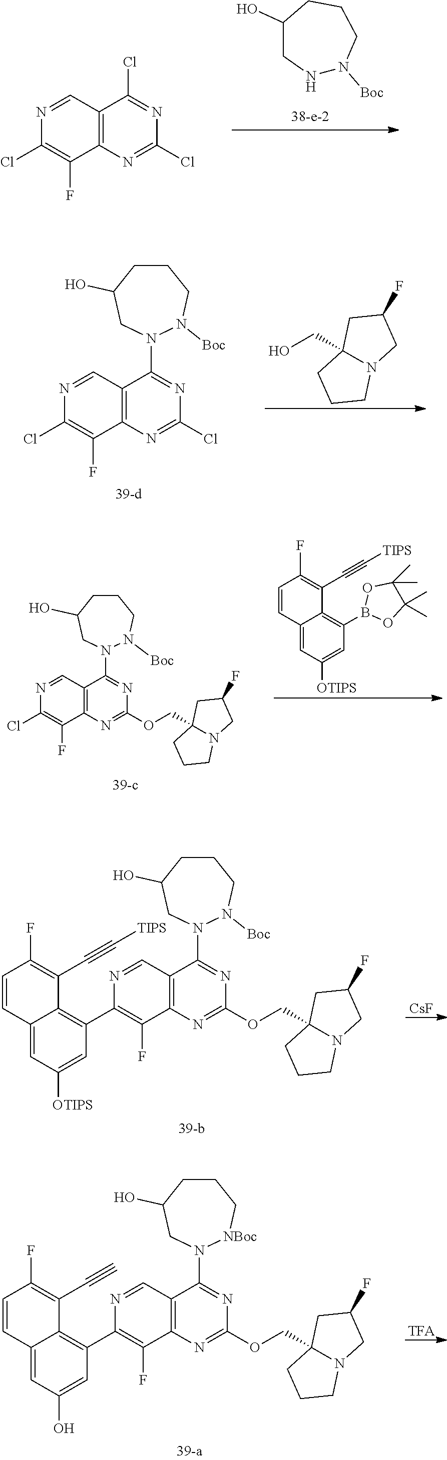

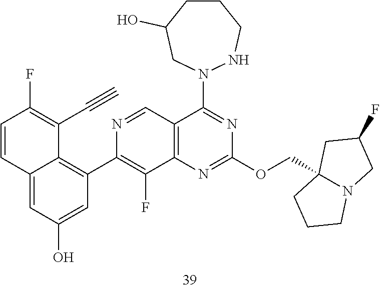 [SMILES: C#Cc1c(F)ccc2cc(O)cc(-c3ncc4c(N5CC(O)CCCN5)nc(OC[C@@]56CCCN5C[C@H](F)C6)nc4c3F)c12]

### Synthesis of Compound 39-d

At room temperature, to a reaction vial were added 2,4,7-trichloro-8-fluoropyrido[4,3-d]pyrimidine (1471 mg, 5.83 mmol) and DCM (30 mL). After degassed and purged with nitrogen, the above mixture was cooled to −78° C. in a dry ice-acetone bath, the above mixture was added DIPEA (1506 mg, 11.65 mmol) and 38-e-2 (420 mg, 1.94 mmol). After addition, the mixture was naturally warmed to room temperature and stirred overnight. Water was added to the reaction mixture, the aqueous phase was extracted with DCM. The organic phases were combined, dried over anhydrous sodium sulfate, concentrated, and the residue was purified by column chromatography (mobile phase: PE/EA 10/0 to 7/3) to give compound 39-d (600 mg, 71%).

### Synthesis of Compound 39-c

At room temperature, to a reaction vial were added 39-d (300 mg, 0.69 mmol), ((2R,7aS)-2-fluorohexahydro-1H-pyrrolizin-7a-yl)methanol (221 mg, 1.39 mmol), 1,4-dioxane (10 mL) and DIPEA (359 mg, 2.78 mmol). After degassed and purged with nitrogen, the reaction mixture was stirred at 90° C. overnight. The reaction mixture was concentrated and the residue was purified by column chromatography (mobile phase: DCM/MeOH 10/0 to 10/1) to give compound 39-c (250 mg, 65%). LC-MS (ESI): m/z 555.9 (M+H)⁺.

### Synthesis of Compound 39-b

To a sealed tube at room temperature were added 39-c (250 mg, 0.45 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (422 mg, 0.68 mmol), cataCXium A Pd G3 (66 mg, 0.09 mmol), potassium phosphate (287 mg, 1.35 mmol), THF (2 mL) and water (0.5 mL). After degassed and purged with nitrogen, the reaction mixture was stirred at 65° C. overnight. The reaction mixture was concentrated and the residue was purified by column chromatography (mobile phase: DCM/MeOH 10/0 to 10/1) to afford compound 39-b (260 mg, 57%). LC-MS (ESI): m/z 1017.9 (M+H)⁺.

### Synthesis of Compound 39-a

39-b (260 mg, 0.26 mmol), DMF (2 mL) and cesium fluoride (388 mg, 2.56 mmol) were added to a reaction vial at room temperature. After degassed and purged with nitrogen, the reaction mixture was stirred for 2 hours at room temperature. The reaction mixture was added water and the aqueous phase was extracted by EA. The organic phases were combined, washed with water, dried over anhydrous sodium sulfate and concentrated to give the crude compound 39-a (400 mg). LC-MS (ESI): m/z 705.9 (M+H)⁺.

### Synthesis of Compound 39

At room temperature, to a reaction vial were added crude compound 39-a (400 mg, 0.26 mmol), DCM (6 mL) and TFA (2 mL). After stirred at room temperature for 1.5 hours, the reaction mixture was neutralized with aqueous sodium bicarbonate and the aqueous phase was extracted with DCM:MeOH=10:1 solution. The organic phases were combined, washed with water, dried over anhydrous sodium sulfate, concentrated and the residue was purified by prep-HPLC to give compound 39 (36 mg, 23%). LC-MS (ESI): m/z 605.8 (M+H)⁺.

### Example 38 Synthetic Route of Compound 40

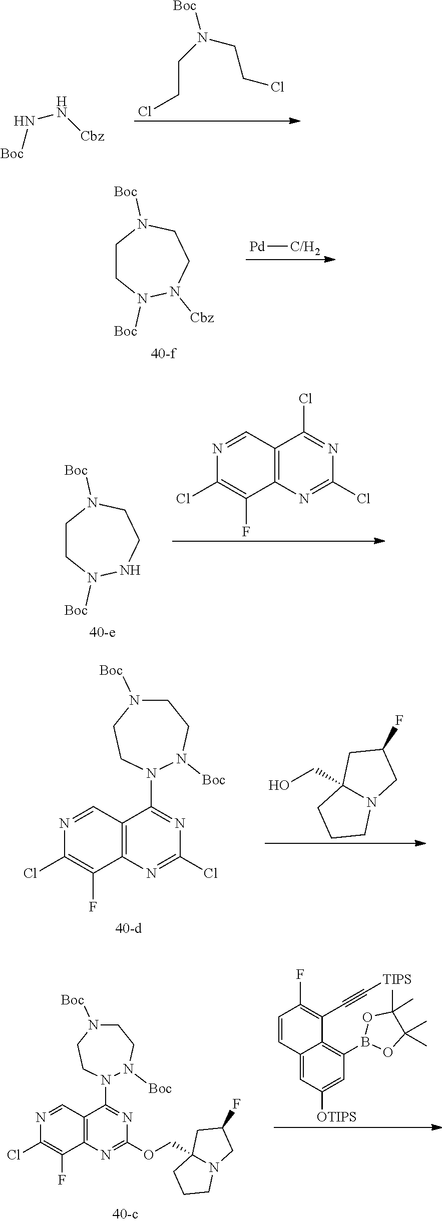

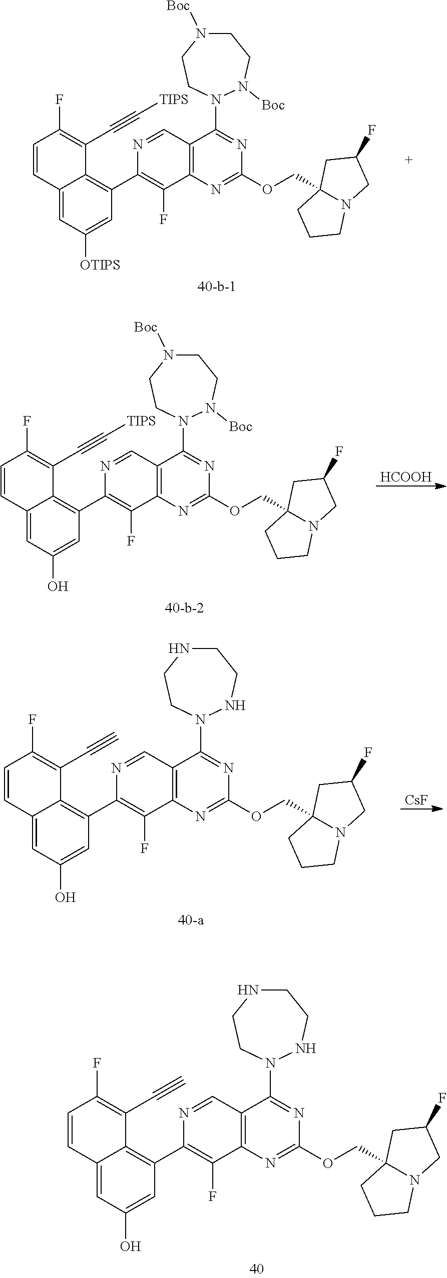

### Synthesis of Compound 40-f

To a solution of 2-(tert-butyl)hydrazine-1,2-dicarboxylic acid-1-benzyl ester (2 g, 7.51 mmol) in DMF (60 mL) was added 60% sodium hydrogen (0.75 g, 18.78 mmol) in batches. After addition, the mixture was stirred at room temperature for 1 h, then was added a solution of tert-butyl N,N-bis(2-chloroethyl)carbamate (1.82 g, 7.51 mmol) in DMF (6 mL) dropwise and the mixture was stirred at 80° C. for 72 hours. The reaction mixture was cooled to room temperature, quenched with ice-cold water. The aqueous phase was extracted with ethyl acetate (100 mL*2), the combined organic phases were washed with brine (50 mL*3), dried over anhydrous sodium sulfate, filtered, concentrated to dryness, and the residue was purified by column chromatography (mobile phase: ethyl acetate: petroleum ether=0-20%) to give compound 40-f (0.7 g, 21%). LC-MS (ESI): m/z 453.3 (M+NH₄)⁺.

### Synthesis of Compound 40-e

To a solution of 40-f (850 mg, 1.95 mmol) in ethanol (50 mL) was added 10% Pd/C (208 mg, 0.19 mmol). After degassed and purged with hydrogen for three times, the mixture was stirred for 2 h at room temperature. Filtered, and the filtrate was concentrated to dryness. The residue was purified by column chromatography (mobile phase: ethyl acetate: petroleum ether=0-30%) to give compound 40-e (506 mg, 86%). LC-MS (ESI): m/z 302.2 (M+H)⁺.

### Synthesis of Compound 40-d

To a solution of 40-e (500 mg, 1.66 mmol) and N,N-diisopropylethylamine (643 mg, 4.98 mmol) in dichloromethane (50 mL) was added 2,4,7-trichloro-8-fluoro-pyrido[4,3-d]pyrimidine (628 mg, 2.49 mmol) in an acetone-dry ice-water bath. The mixture was naturally warmed to room temperature and stirred overnight. The next day, water was added to the reaction mixture. The aqueous phase was extracted with dichloromethane (80 mL*2), the combined organic phases were dried over anhydrous sodium sulfate, filtered, concentrated to dryness, and the residue was purified by column chromatography (mobile phase: ethyl acetate: petroleum ether=0-20%) to give compound 40-d (742 mg, 86%). LC-MS (ESI): m/z 517.4 (M+H)⁺.

### Synthesis of Compound 40-c

To a solution of 40-d (740 mg, 1.43 mmol) in dichloromethane (15 mL) was added ((2R,7aS)-2-fluorohexahydro-1H-pyrrolozin-7a-yl)methanol (273 mg, 1.72 mmol) in an ice-water bath, followed by addition of sodium tert-butanolate (344 mg, 3.58 mmol), and the mixture was stirred for 1 h at this temperature. Then water was added and the aqueous phase was extracted with dichloromethane (80 mL*2), the combined organic phases were washed with brine, dried over anhydrous sodium sulfate, filtered, concentrated to dryness, and the crude was purified by column chromatography (mobile phase: dichloromethane: methanol=10:1/dichloromethane, 0-30%) to give compound 40-c (751 mg, 82%). LC-MS (ESI): m/z 640.8 (M+H)⁺.

### Synthesis of Compound 40-b

To a microwave tube were added 40-c (100 mg, 0.16 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (146 mg, 0.23 mmol), methanesulfonic acid [n-butylbis(1-adamantyl ) phosphine](2-amino-1,1′-biphenyl-2-yl)palladium(II) (11 mg, 0.016 mmol), potassium phosphate (99 mg, 0.47 mmol), tetrahydrofuran (10 mL) and water (1 mL). After degassed and purged with nitrogen for three times, the microwave tube was sealed and stirred at 65° C. overnight. The reaction mixture was concentrated to dryness, and the crude product was purified by column chromatography (mobile phase: dichloromethane: methanol=10:1/dichloromethane, 0-50%) to give compound 40-b-1 (123 mg, 71%), LC-MS (ESI): m/z 1103.0 (M+H)+, and 40-b-2 (40 mg, 27%, dropping one TIPS group), LC-MS (ESI): m/z 946.4 (M+H)⁺.

### Synthesis of Compound 40-a

To a mixture of 40-b-1 and 40-b-2 (163 mg, 0.15 mmol) was added formic acid (10 mL) at room temperature and the mixture was stirred at room temperature overnight. The reaction mixture was concentrated to dryness, and the residue was added saturated sodium bicarbonate, extracted with ethyl acetate (50 mL*2). The organic phase was dried and concentrated to dryness to give compound 40-a (117 mg, 140%), which was used directly in the next step without purification. LC-MS (ESI): m/z 746.4 (M+H).

### Synthesis of Compound 40

Cesium fluoride (238 mg, 1.57 mmol) was added to a solution of 40-a (117 mg, 0.16 mmol) in N,N-dimethylformamide (10 mL) at room temperature and the mixture was stirred at room temperature overnight. A large amount of The reaction mixture was poured water, and the aqueous phase was extracted with ethyl acetate (50 mL*2). The organic phase was washed with brine (50 mL*3), concentrated to dryness, and the residue was purified by prep- HPLC (ammonium bicarbonate) to give compound 40 (23 mg, 25%). LC-MS (ESI): m/z 590.2 (M+H)⁺.

### Example 39 Synthetic Route of Compound 41

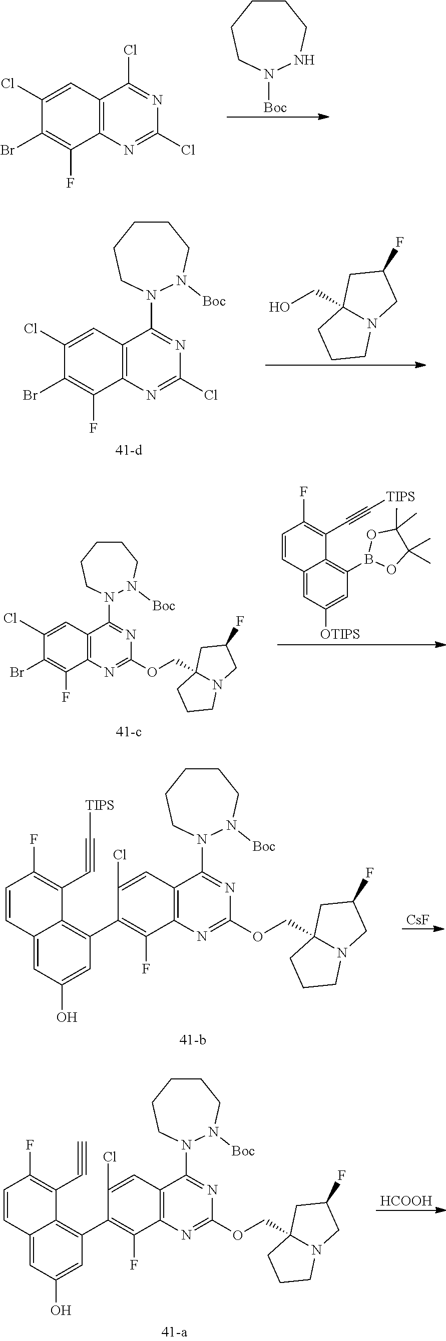

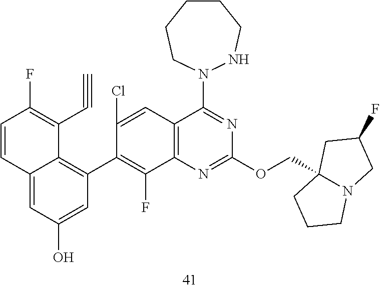 [SMILES: C#Cc1c(F)ccc2cc(O)cc(-c3c(Cl)cc4c(N5CCCCCN5)nc(OC[C@@]56CCCN5C[C@H](F)C6)nc4c3F)c12]

### Synthesis of Compound 41-d

To a solution of 7-bromo-2,4,6-trichloro-8-fluoroquinazoline (1 g, 3.03 mmol) and N,N-diisopropylethylamine (1173 mg, 9.08 mmol) in tetrahydrofuran (50 mL) in an ice-water bath was added tert-butyl 1,2-diazepine-1-carboxylate (606 mg, 3.03 mmol), and the mixture was naturally warmed to room temperature and stirred overnight. The next day, water was added to the reaction mixture and the aqueous phase was extracted with ethyl acetate (100 mL*2). The combined organic phases were washed with brine (10 mL*2), dried over anhydrous sodium sulfate, filtered, concentrated to dryness, and the residue was purified by column chromatography (mobile phase: ethyl acetate: petroleum ether=0-10%) to give compound 41-d (1350 mg, 90%). LC-MS (ESI): m/z 493.1 (M+H)⁺.

### Synthesis of Compound 41-c

To a solution of 41-d (300 mg, 0.61 mmol) in dichloromethane (30 mL) was added ((2R,7aS)-2-fluorohexahydro-1H-pyrrolozin-7a-yl)methanol (232 mg, 1.46 mmol) in an ice-water bath, followed by slow addition of sodium tert-butanolate (146 mg, 1.52 mmol), and the reaction mixture was stirred at room temperature for 2 hours. Water was added to the reaction mixture and the aqueous phase was extracted with dichloromethane (80 mL*2). The combined organic phases were washed with brine, dried over anhydrous sodium sulfate, filtered, concentrated to dryness, and the crude was purified by column chromatography (mobile phase: ethyl acetate: petroleum ether=0-100%) to give compound 41-c (176 mg, 47%). LC-MS (ESI): m/z 616.2 (M+H)⁺.

### Synthesis of Compound 41-b

To a microwave tube were added 41-c (80 mg, 0.13 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (162 mg, 0.26 mmol), palladium tetratriphenylphosphine (15 mg. 0.013 mmol), sodium carbonate (41 mg, 0.39 mmol), 1,4-dioxane (15 mL) and water (1.5 mL). After degassed and purged with nitrogen for three times, the microwave tube was sealed, stirred at 100° C. overnight. The reaction mixture was concentrated to dryness and the crude product was purified by column chromatography (mobile phase: dichloromethane: methanol=10:1/dichloromethane, 0-100%) to give compound 41-b (40 mg, 35%). LC-MS (ESI): m/z 878.3 (M+H)⁺.

### Synthesis of Compound 41-a

To a solution of 41-b (40 mg, 0.046 mmol) in N,N-dimethylformamide (6 mL) was added cesium fluoride (69 mg, 0.46 mmol) at room temperature, and the mixture was stirred for 1 h at room temperature. A large amount of The reaction mixture was poured water, the aqueous phase was extracted with ethyl acetate (50 mL*2). The organic phase was washed with brine (30 mL*3), concentrated to dryness, and the residue was purified by column chromatography (mobile phase: dichloromethane: methanol=10:1/dichloromethane, 0-100%) to give compound 41-a (13 mg, 40%).

LC-MS (ESI): m/z 722.3 (M+H)⁺.

### Synthesis of Compound 41

To 41-a (13 mg, 0.018 mmol) was added formic acid (6 mL) at room temperature and the mixture was stirred at room temperature overnight. The reaction mixture was concentrated to dryness at room temperature, purified by prep-HPLC (ammonium bicarbonate) and lyophilized to give compound 41 (2.5 mg, 22%). LC-MS (ESI): m/z 622.2 (M+H)⁺.

### Example 40 Synthetic Route of Compound 42

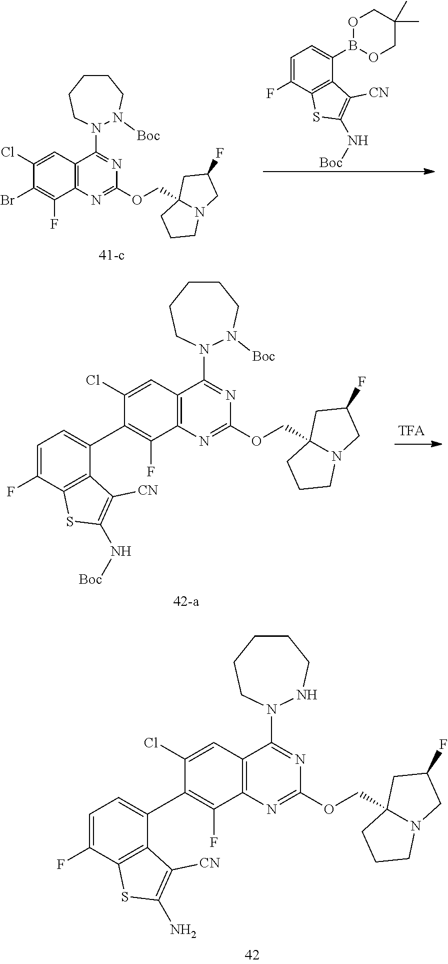 [SMILES: CC(C)(C)OC(=O)N1CCCCCN1c1nc(OC[C@@]23CCCN2C[C@H](F)C3)nc2c(F)c(Br)c(Cl)cc12 ; CC1(C)COB(c2ccc(F)c3sc(NC(=O)OC(C)(C)C)c(C#N)c23)OC1 ; CC(C)(C)OC(=O)Nc1sc2c(F)ccc(-c3c(Cl)cc4c(N5CCCCCN5C(=O)OC(C)(C)C)nc(OC[C@@]56CCCN5C[C@H](F)C6)nc4c3F)c2c1C#N ; N#Cc1c(N)sc2c(F)ccc(-c3c(Cl)cc4c(N5CCCCCN5)nc(OC[C@@]56CCCN5C[C@H](F)C6)nc4c3F)c12]

### Synthesis of Compound 42-a

To a microwave tube were added 41-c (100 mg, 0.16 mmol), tert-butyl (3-cyano-4-(5,5-dimethyl-1,3,2-dioxaborinan-2-yl)-7-fluorobenzo[b]thiophen-2-yl)carbamate (197 mg, 0.49 mmol), bis(diphenylphosphine phenyl ether) palladium(II) dichloride (12 mg, 0.016 mmol), cesium carbonate (264 mg, 0.81 mmol) and toluene (15 mL). After degassed and purged with nitrogen for three times, the microwave tube was sealed, stirred at 100° C. for 1.5 hours. The reaction mixture was concentrated to dryness and the crude product was purified by column chromatography (mobile phase: dichloromethane: methanol=10:1/dichloromethane, 0-60%) to give compound 42-a (153 mg, 96%). LC-MS (ESI): m/z 828.5 (M+H)⁺.

### Synthesis of Compound 42

Trifluoroacetic acid (2 mL) was added to a solution of 42-a (50 mg, 0.060 mmol) in dichloromethane (6 mL) at room temperature and the mixture was stirred for 1 h at room temperature. The reaction mixture was concentrated to dryness, purified by prep-HPLC (ammonium bicarbonate) and lyophilized to give compound 42 (9 mg, 24%). LC-MS (ESI): m/z 628.5 (M+H)⁺; ¹H NMR (400M, CD₃OD) δ 9.56 (1H, d, J=1.6 Hz), 7.21-7.15 (1H, m), 7.02 (1H, t, J=9.2 Hz), 5.40-5.21 (1H, m), 4.60(3H, bs), 4.32-4.06 (4H, m), 3.39-2.96 (6H, m), 2.42-2.08 (3H, m), 2.05-1.94 (4H, m), 1.94-1.84 (1H, m), 1.82-1.67 (4H, m).

### Example 41 Synthetic Route of Compound 43

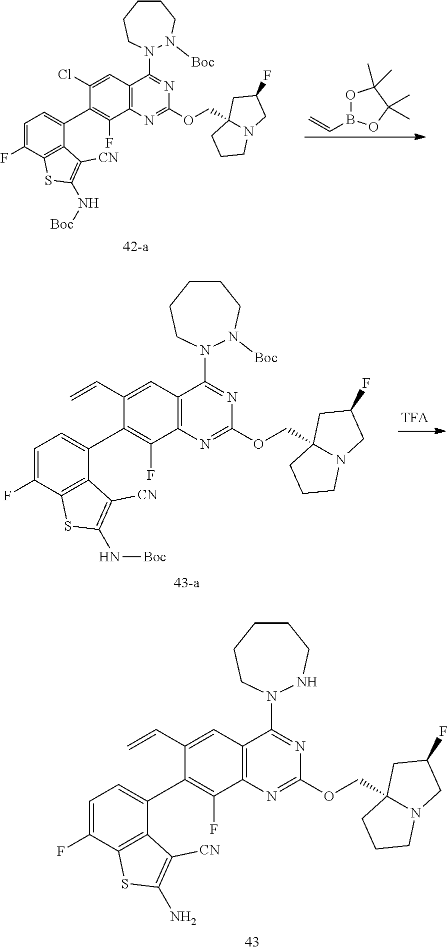

### Synthesis of Compound 43-a

To a microwave tube were added 42-a (140 mg, 0.17 mmol), methanesulfonic acid (2-dicyclohexylphosphino-2′,6′-dimethoxy-1,1′-biphenyl)(2′-amino-1,1′-biphenyl-3-yl)palladium(II) (53 mg, 0.068 mmol), potassium phosphate (194 mg, 0.91 mmol), 1,4-dioxane (12 mL) and water (2 mL). After degassed and purged with nitrogen for three times, the microwave tube was sealed, and was added vinylboronic acid pinacol ester (135 mg, 0.88 mmol) by syringe, then stirred at 150° C. for 45 min. The reaction mixture was concentrated to dryness and the crude product was purified by column chromatography (mobile phase: dichloromethane: methanol=10:1/dichloromethane, 0-100%) to afford compound 43-a (110 mg, 79%) as a mixture of compounds with 2 Boc, 1 Boc and no Boc.

### Synthesis of Compound 43

Trifluoroacetic acid (3 mL) was added to a solution of 43-a (70 mg, 0.085 mmol) in dichloromethane (9 mL) at room temperature, and the mixture was stirred for 1 h at room temperature. The reaction mixture was concentrated to dryness, dissolved in methanol, neutralized to pH 8 with saturated sodium bicarbonate solution, concentrated to dryness, and the residue was purified by column chromatography (mobile phase: ammonia methanol: dichloromethane=0-10%) to give compound 43 (30 mg, 57%). LC-MS (ESI): m/z 620.5 (M+H)⁺.

### Example 42 Synthetic Route of Compound 44

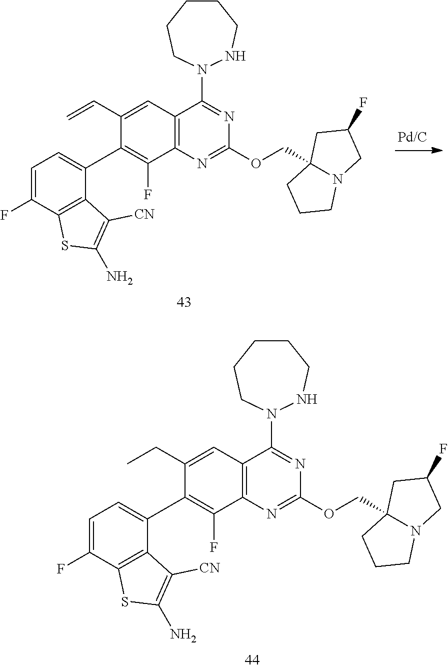 [SMILES: C=Cc1cc2c(N3CCCCCN3)nc(OC[C@@]34CCCN3C[C@H](F)C4)nc2c(F)c1-c1ccc(F)c2sc(N)c(C#N)c12 ; CCc1cc2c(N3CCCCCN3)nc(OC[C@@]34CCCN3C[C@H](F)C4)nc2c(F)c1-c1ccc(F)c2sc(N)c(C#N)c12]

### Synthesis of Compound 44

To a solution of 43 (25 mg, 0.040 mmol) in ethanol (20 mL) was added 10% Pd/C (22 mg, 0.020 mmol) at room temperature. After degassed and purged with hydrogen for three times, the mixture was stirred for 4 h at room temperature. The reaction mixture was filtered and the filtrate was concentrated to dryness, purified by prep-HPLC (ammonium bicarbonate) and lyophilized to give compound 44 (9 mg, 36%). LC-MS (ESI): m/z 622.8 (M+H)⁺; ¹H NMR (400M, CD₃OD) δ 9.29 (1H, s), 7.18-7.11 (1H, m), 7.05-6.97 (1H, m), 5.41-5.21 (1H, m), 4.32-4.05 (4H, m), 3.39-2.97 (6H, m), 2.56-2.09 (5H, m), 2.06-1.66 (9H, m), 1.07 (3H, t, J=7.6 Hz).

### Example 43 Synthetic Route of Compound 45

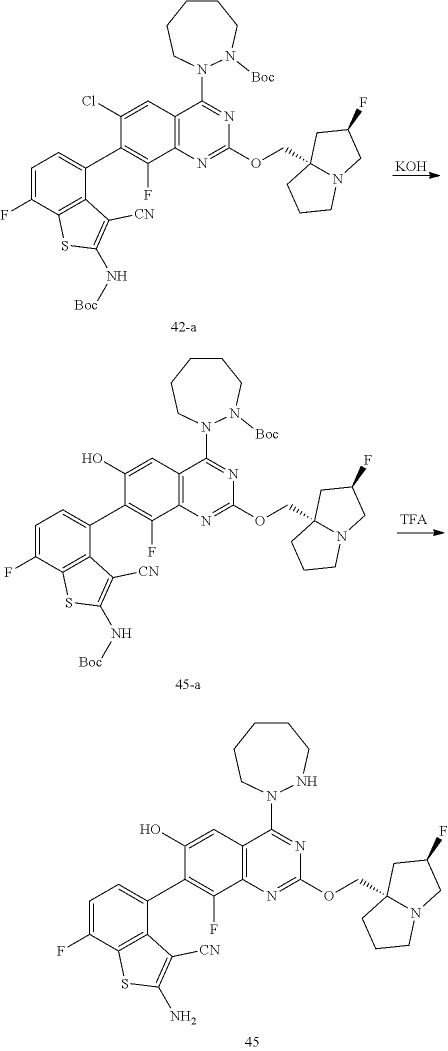

### Synthesis of Compound 45-a

A reaction vial charged with 42-a (100 mg, 0.12 mmol), potassium hydroxide (339 mg, 6.04 mmol), methanesulfonic acid (2-di-tert-butylphosphino-2′,4′,6′-triisopropyl-1,1′-biphenylyl)(2′-amino-1,1′-biphenylyl-2-yl)palladium(II) (10 mg, 0.012 mmol), 1,4-dioxane (20 mL), water (10 mL) was degassed and purged with nitrogen for three times, then stirred at 100° C. for 2 days. The reaction mixture was concentrated to dryness and the crude product was purified by column chromatography (mobile phase: dichloromethane: methanol=10:1/dichloromethane, 0-100%) to give compound 45-a (17 mg, 17%). LC-MS (ESI): m/z 810.4 (M+H)⁺.

### Synthesis of Compound 45

Trifluoroacetic acid (1 mL) was added to a solution of 45-a (17 mg, 0.021 mmol) in dichloromethane (3 mL) at room temperature and the mixture was stirred for 1 h at room temperature. The reaction mixture was concentrated to dryness, purified by prep-HPLC (ammonium bicarbonate) and lyophilized to give compound 45 (2 mg, 17%). LC-MS (ESI): m/z 610.3 (M+H)⁺.

### Example 44 Synthetic Route of Compound 46

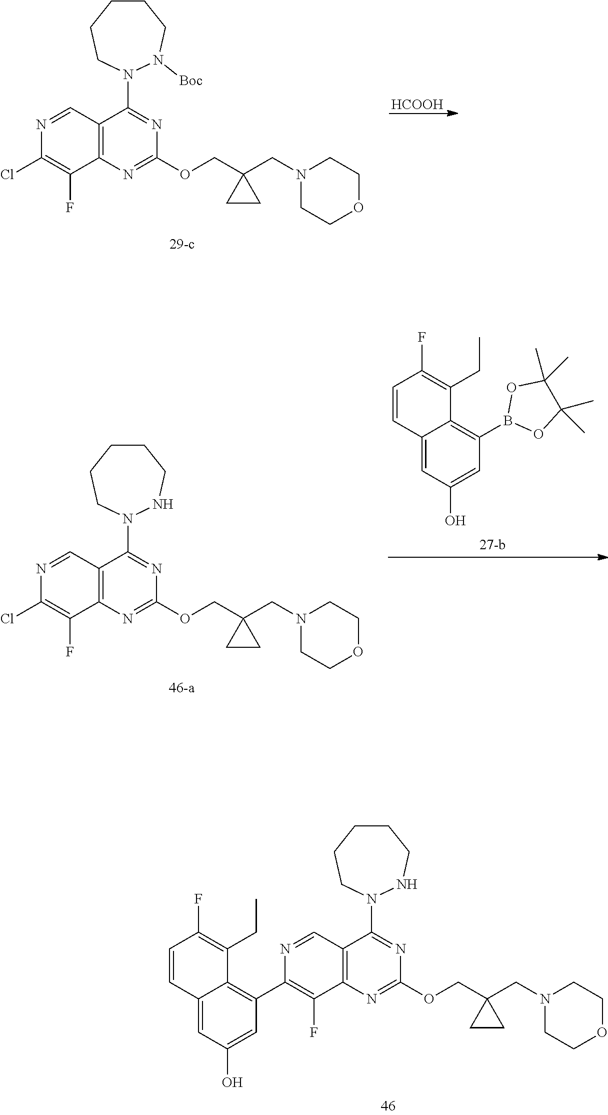 [SMILES: CC(C)(C)OC(=O)N1CCCCCN1c1nc(OCC2(CN3CCOCC3)CC2)nc2c(F)c(Cl)ncc12 ; "HCOOH" ; Fc1c(Cl)ncc2c(N3CCCCCN3)nc(OCC3(CN4CCOCC4)CC3)nc12 ; CCc1c(F)ccc2cc(O)cc(B3OC(C)(C)C(C)(C)O3)c12 ; CCc1c(F)ccc2cc(O)cc(-c3ncc4c(N5CCCCCN5)nc(OCC5(CN6CCOCC6)CC5)nc4c3F)c12]

### Synthesis of Compound 46-a

To a reaction flask were added 29-c (160 mg, 0.29 mmol) and formic acid (10 mL) and the resulting mixture was stirred at room temperature overnight. The reaction mixture was concentrated to dryness, the residue was added dichloromethane, and was adjusted to pH 8 with saturated sodium bicarbonate solution. The aqueous phase was extracted with dichloromethane (40 mL*2). The organic phase was concentrated to dryness and the residue was purified by column chromatography (mobile phase: methanol: dichloromethane=0-10%) to give compound 46-a (97 mg, 74%). LC-MS (ESI): m/z 451.2 (M+H)*.

### Synthesis of Compound 46

To a microwave tube were added 46-a (45 mg, 0.10 mmol), 27-b (47 mg, 0.15 mmol), [n-butylbis(1-adamantyl)phosphine](2-amino-1,1′-biphenyl-2-yl)palladium(II) (7 mg, 0.01 mmol), potassium phosphate (106 mg, 0.50 mmol), tetrahydrofuran (15 mL) and water (1.5 mL). The reaction mixture was degassed and purged with nitrogen for three times, then sealed and stirred at 65° C. overnight. Water was added to the reaction mixture and the aqueous phase was extracted with ethyl acetate (50 mL*2), the combined organic phases were dried over anhydrous sodium sulfate, filtered, concentrated to dryness, purified twice by prep-HPLC (first trifluoroacetic acid, then ammonium bicarbonate), lyophilized to give compound 46 (5 mg, 8%). LC-MS (ESI): m/z 605.3 (M+H)⁺.

### Example 45 Synthetic Route of Compound 47

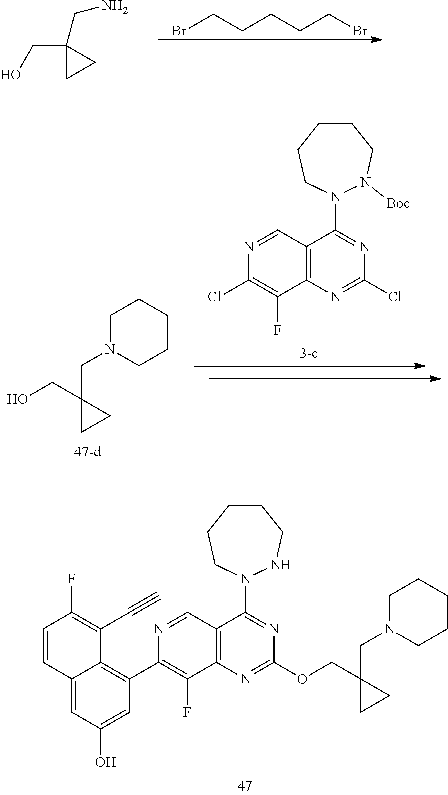 [SMILES: NCC1(CO)CC1 ; BrCCCCCBr ; OCC1(CN2CCCCC2)CC1 ; CC(C)(C)OC(=O)N1CCCCCN1c1nc(Cl)nc2c(F)c(Cl)ncc12 ; C#Cc1c(F)ccc2cc(O)cc(-c3ncc4c(N5CCCCCN5)nc(OCC5(CN6CCCCC6)CC5)nc4c3F)c12]

### Synthesis of Compound 47-d

A reaction vial charged with [1-(aminomethyl)cyclopropyl]methanol (1 g, 9.89 mmol), acetonitrile (30 mL), 1,5-dibromopentane (2.39 g, 10.38 mmol) and potassium carbonate (3.56 g, 25.70 mmol) was stirred at room temperature overnight. The reaction mixture was concentrated to dryness and the residue was purified by column chromatography (mobile phase: ammonia methanol: dichloromethane=0-10%) to give compound 47-d (1 g, 60%). LC-MS (ESI): m/z 170.2 (M+H)⁺.

### Synthesis of Compound 47

Referring to compound synthetic route of compound 29, compound 47 (11 mg, 13%) was synthesized using 47-d instead of (1-(morpholinomethyl)cyclopropyl)methanol, with 3-c as material.

LC-MS (ESI): m/z 599.3 (M+H)⁺.

### Example 46 Synthetic Route of Compound 48

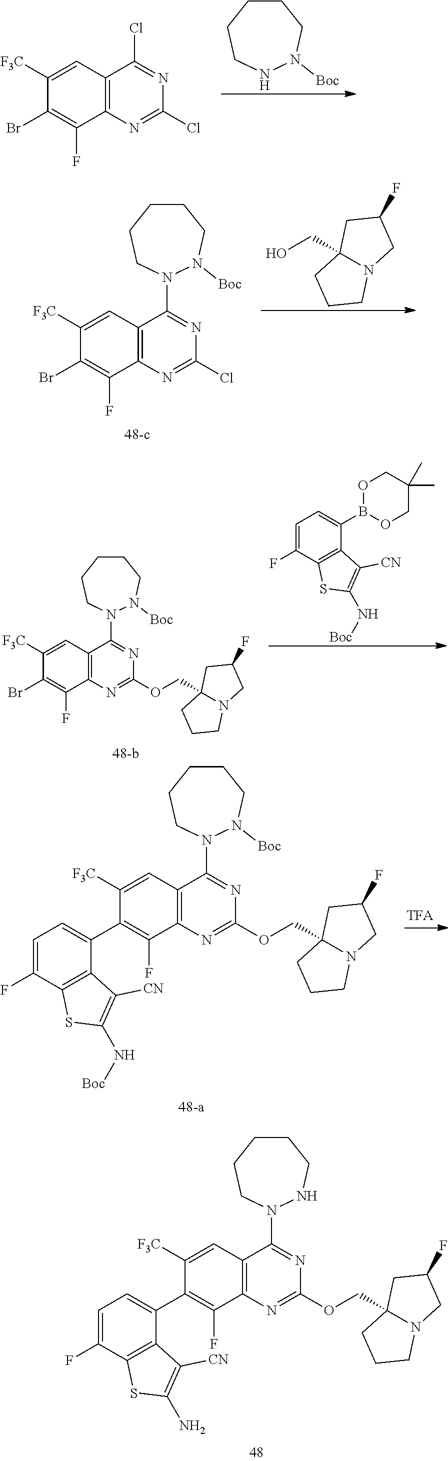

### Synthesis of Compound 48-c

To a solution of 7-bromo-2,4-dichloro-8-fluoro-6-(trifluoromethyl)quinazoline (1 g, 2.75 mmol) and N,N-diisopropylethylamine (1065 mg, 8.24 mmol) in tetrahydrofuran (50 mL) in an ice-water bath was added tert-butyl 1,2-diazepine-1-carboxylate (550 mg, 2.75 mmol). The mixture was naturally warmed to room temperature and stirred overnight. The next day, water was added and the aqueous phase was extracted with ethyl acetate (100 mL*2), the combined organic phases were washed with brine (10 mL*2), dried over anhydrous sodium sulfate, filtered, concentrated to dryness, and purified on a silica column (mobile phase: ethyl acetate: petroleum ether=0-10%) to afford 48-c (1270 mg, 88%). LC-MS (ESI): m/z 527.1 (M+H)⁺.

### Synthesis of Compound 48-b

To a solution of 48-c (1200 mg, 2.27 mmol) in tetrahydrofuran (25 mL) and N,N-dimethylformamide (25 mL) at room temperature was added ((2R,7aS)-2-fluorohexahydro-1H-pyrrolizin-7a-yl)methanol (1086 mg, 6.82 mmol), cesium carbonate (2223 mg, 6.82 mmol) and DABCO (1020 mg, 9.10 mmol), and the mixture was stirred at room temperature overnight. Water was added to the reaction mixture and the aqueous phase was extracted with ethyl acetate (100 mL*2), the combined organic phases were washed with brine (50 mL*3), dried over anhydrous sodium sulfate, filtered, concentrated to dryness, and the crude was purified by column chromatography (mobile phase: methanol: dichloromethane=0-10%) to give compound 48-b (649 mg, 44%). LC-MS (ESI): m/z 650.3 (M+H)⁺.

### Synthesis of Compound 48-a

To a microwave tube were added 48-b (60 mg, 0.092 mmol), tert-butyl (3-cyano-4-(5,5-dimethyl-1,3,2-dioxaborinan-2-yl)-7-fluorobenzo[b]thiophen-2-yl)carbamate (75 mg, 0.18 mmol), bis(diphenylphosphine phenyl ether)palladium(II) dichloride (7 mg, 0.0092 mmol), cesium carbonate (150 mg, 0.46 mmol) and toluene (10 mL). After degassed and purged with nitrogen for three times, the microwave tube was sealed, stirred at 100° C. for 1.5 hours. The reaction mixture was concentrated to dryness and the crude product was purified by column chromatography (mobile phase: methanol: dichloromethane=0-10%) to give compound 48-a (60 mg, 75%). LC-MS (ESI): m/z 862.6 (M+H)⁺.

### Synthesis of Compound 48

Trifluoroacetic acid (2 mL) was added to a solution of 48-a (60 mg, 0.070 mmol) in dichloromethane (6 mL) at room temperature and the mixture was stirred for 2 hours at room temperature. The reaction mixture was concentrated to dryness, purified by prep-HPLC (ammonium bicarbonate) and lyophilized to give compound 48 (14 mg, 30%). LC-MS (ESI): m/z 662.2 (M+H)⁺; ¹H NMR (400 M, CD₃OD) δ 9.93 (1H, s), 7.22-7.15 (1H, m), 7.03-6.95 (1H, m), 5.41-5.19 (1H, m), 4.60(1H, bs), 4.27 (1H, d, J=10.4 Hz), 4.23-4.06 (3H, m), 3.27-2.95 (5H, m), 2.41-2.08 (3H, m), 2.06-1.67 (9H, m).

### Example 47 Synthetic Route of Compound 49

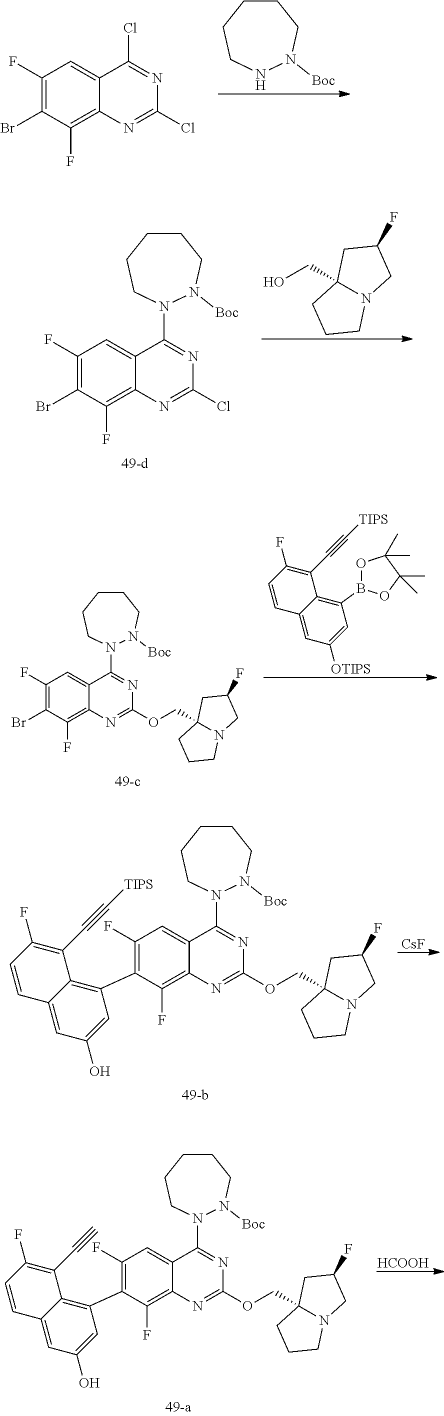

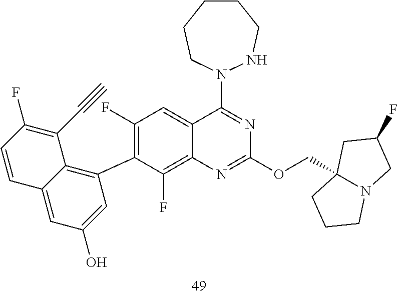 [SMILES: C#Cc1c(F)ccc2cc(O)cc(-c3c(F)cc4c(N5CCCCCN5)nc(OC[C@@]56CCCN5C[C@H](F)C6)nc4c3F)c12]

### Synthesis of Compound 49-d

A reaction vial charged with 7-bromo-2,4-dichloro-6,8-difluoroquinazoline (400 mg, 1.27 mmol), tetrahydrofuran (100 mL), DIPEA (444 mg, 3.44 mmol) and tert-butyl 1,2-diazepine-1-carboxylate (383 mg, 1.91 mmol) was stirred for 12 h at room temperature. The reaction mixture was concentrated at room temperature and saturated sodium bicarbonate solution (100 mL) was added to the residue. The aqueous phase was extracted with dichloromethane (100 mL*3). The organic phase was concentrated and the residue was purified by column chromatography (PE/EA=10/1) to give compound 49-d (396 mg, 65%). LC-MS (ESI): m/z=477.6 (M+H)⁺.

### Synthesis of Compound 49-c

A reaction vial charged with 49-d (400 mg, 0.84 mmol), tetrahydrofuran (15 mL), DABCO (376 mg, 3.35 mmol), DMF (15 mL), ((2R,7aS)-2-fluorohexahydro-1H-pyrrolylazin-7a-yl)methanol (400 mg, 2.51 mmol), cesium carbonate (818 mg. 2.51 mmol) was stirred overnight under protection of nitrogen. Water (20 mL) was added to the reaction mixture and the aqueous phase was extracted with ethyl acetate (50 mL*3). The organic phase was concentrated and the residue was purified by column chromatography (MeOH/DCM=1/10) to afford compound 49-c (380 mg, 76%). LC-MS (ESI): m/z=600.1(M+H)⁺.

### Synthesis of Compound 49-b

A reaction vial charged with 49-c (100 mg, 0.17 mmol), 1,4-dioxane (3 mL), water (0.5 mL), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (208 mg. 0.33 mmol), sodium carbonate (53 mg, 0.50 mmol) and palladium tetrakis(triphenylphosphine) (38 mg, 0.03 mmol) was degassed and purged with nitrogen for three times and the reaction mixture was stirred in an oil bath prestirred at 100° C. for 12 hours. The reaction mixture was concentrated to dryness and the residue was purified by column chromatography (mobile phase: methanol/dichloromethane=1/30; 1/10) to afford compound 49-b (79 mg, 55%). LC-MS (ESI): m/z=862.5(M+H)⁺.

### Synthesis of Compound 49-a

To a reaction flask were added 49-b (79 mg, 0.09 mmol), DMF (6 mL) and cesium fluoride (230 mg, 1.51 mmol). The resulting mixture was stirred at room temperature for 6 hours, then was added water (50 mL), filtered, and the filter cake was dried to give compound 49-a (59 mg, 91%). LC-MS (ESI): m/z=706.3 (M+H)⁺.

### Synthesis of Compound 49

A reaction vial charged with 49-a (59 mg, 0.08 mmol) and formic acid (5 mL) was stirred at room temperature for 12 hours. The reaction mixture was concentrated at room temperature and saturated sodium bicarbonate (20 mL) was added to the residue and the aqueous phase was extracted with ethyl acetate (20 mL*3). The organic phase was concentrated and the residue was purified by prep-HPLC to afford compound 49 (20.6 mg, 41%), LC-MS (ESI): m/z 606.3(M+H)⁺; ¹H NMR (400 MHz, DMSO-d₆): δ 10.21 (1H, s), 9.08 (1H, d, J=12.4 Hz), 7.98 (1H, t, J=6.4 Hz), 7.48 (1H, t, J=9.2 Hz), 7.40 (1H, s), 7.15 (1H, s), 5.68 (1H, t, J=6.0 Hz), 5.29 (1H, d, J=54.8 Hz), 4.16-3.90 (4H, m), 3.94 (1H, s), 3.18-2.95 (5H, m), 2.93-2.78 (1H, m), 2.25-2.00 (4H, m), 1.98-1.74 (5H, m), 1.73-1.60 (3H, m).

### Example 48 Synthetic Route of Compound 50

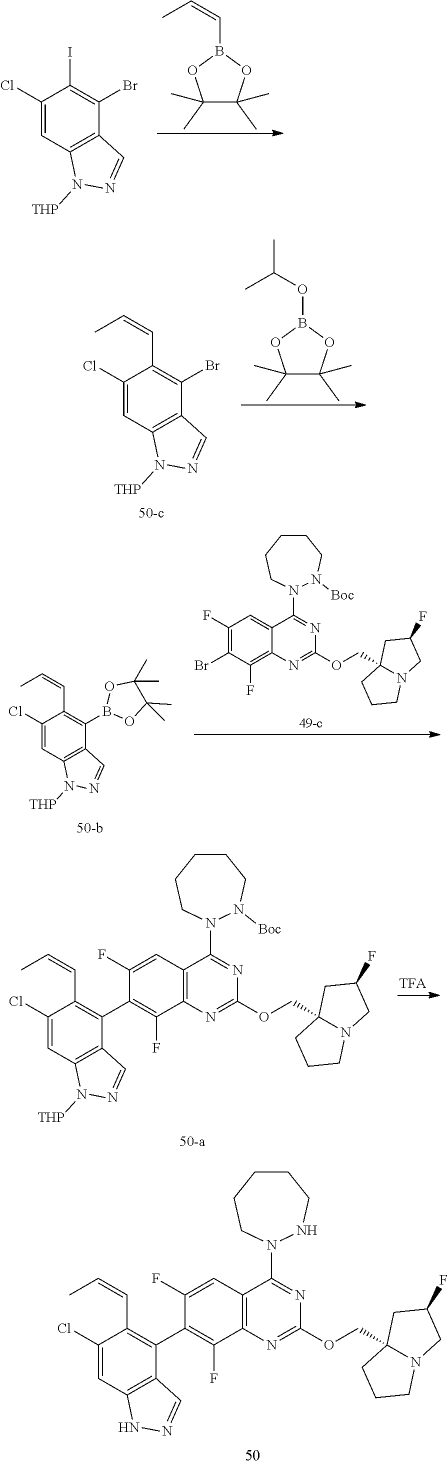

### Synthesis of Compound 50-c

To a reaction flask were added 4-bromo-6-chloro-5-iodo-h-(tetrahydro-2H-pyran-2-yl)-1H-indazole (1000 mg, 2.27 mmol), 1,4-dioxane (12 mL), water (3 mL), ((Z)-1-propenyl)-pinacol ester of borate (500 mgs 2.98 mmol) potassium phosphate (1700 mg. 8.01 mmol) and PdCl₂(dppf)CH₂C 2 (170 mg, 0.21 mmol). After degassed and purged with nitrogens for three times, the reaction flask was placed in an oil bath prestirred at 100° C. and stirred for 3 hours. The reaction mixture was concentrated to dryness and the residue was purified by column chromatography (mobile phase: PE-EA=100/0; 100/5) to give compound 50-c (380 mg, 47%).

### Synthesis of Compound 50-b

To a reaction vial were added 50-c (360 mg, 1.01 mmol) and tetrahydrofuran (10 mL). The resulting mixture was stirred at −78° C. for 10 min, then was slowly added n-Butyllithium solution (2.5M, 0.6 mL, 1.50 mmol) dropwise. After addition, the mixture was stirred at the same temperature for 30 minutes, then was slowly added isopropanol pinacol borate (283 mg, 1.52 mmol) and stirred at -78° C. for 60 minutes. Saturated ammonium chloride (30 m L) was added to the reaction mixture and the aqueous phase was extracted with ethyl acetate (20 mL*3). The organic phase was concentrated and the residue was purified by column chromatography (PE/EA=100/0; 100/3) to afford compound 50-b (210 mg, 52%). LC-MS (ESI): m/z 403.1(M+H)⁺.

### Synthesis of Compound 50-a

To a reaction flask were added 49-c (149 mg, 0.25 mmol), 1,4-dioxane (10 mnL), water (2 mL), 50-b (100 mg, 0.25 mmol), cesium fluoride (113 mg, 0.75 mmol) and PdCl₂ (dtbpf) (30 mg, 0.05 mmol). After degassed and purged with nitrogen for three times, the reaction flask was placed in an oil bath pre-heated 100° C. and stirred in for 2 hours. The reaction mixture was concentrated to dryness and the residue was purified by column chromatography (mobile phase: MeOH/DCM=1/100; 1/10) to afford compound 50-a (180 mg, 91%). LC-MS (ESI): m/z=796.4 (M+H)⁺.

### Synthesis of Compound 50

A reaction vial charged with 50-a (80 mg, 0.10 mmol), dichloromethane (3 mL) and trifluoroacetic acid (1 mL) was stirred at room temperature for 3 hours. The reaction mixture was concentrated to dryness, and the residue was added saturated sodium bicarbonate (20 mL) and the aqueous phase was extracted with ethyl acetate (20 mL*3). The organic phase was concentrated and the residue was purified by column chromatography (mobile phase: DCM/MeOH=30/1; 10/1) to afford compound 50 (27.8 mg, 45%). LC-MS (ESI): m/z=612.5 (M+H)⁺.

### Example 49 Synthetic Route of Compound 51

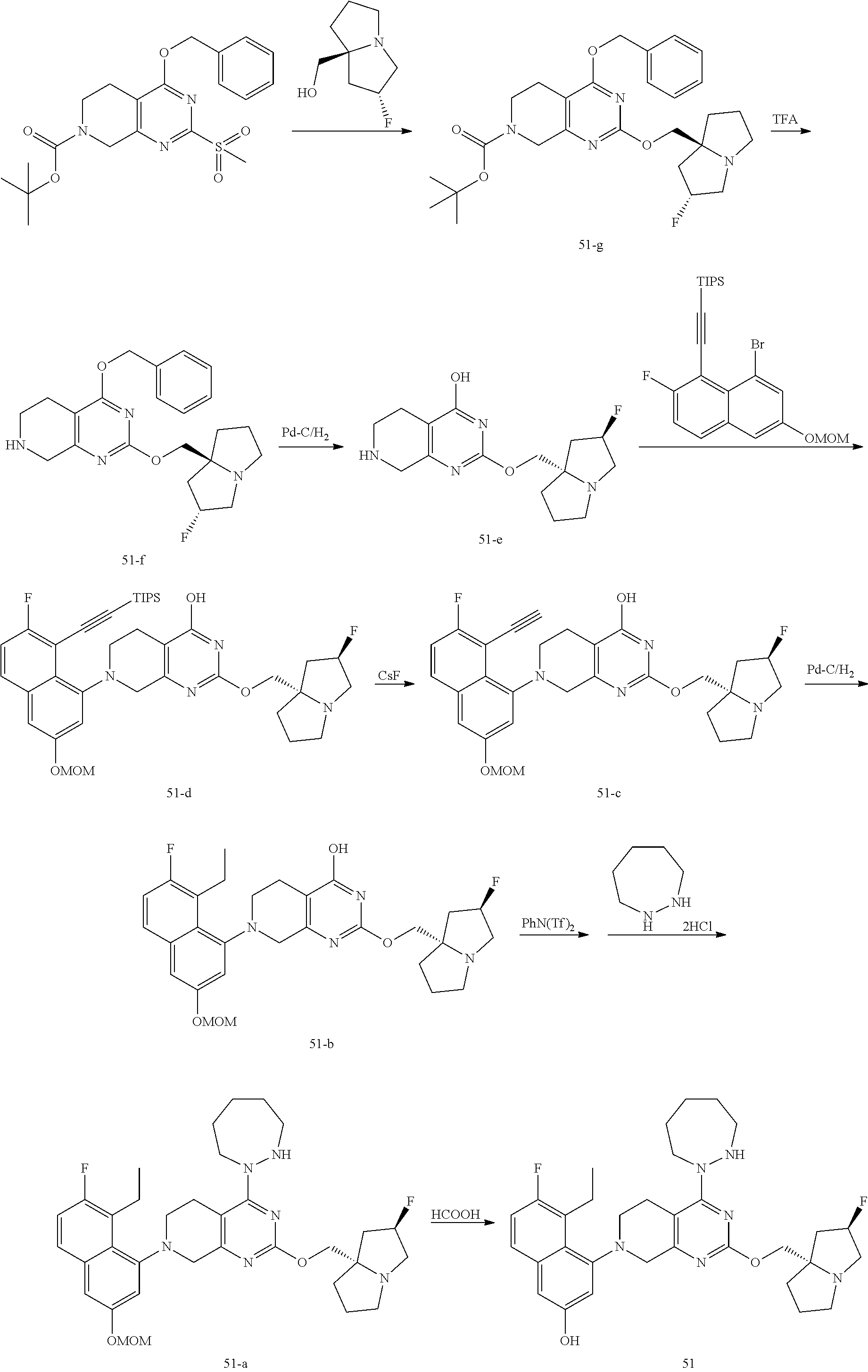

### Synthesis of compounds 51-g PP-124,C₃

A reaction vial charged with tert-butyl 4-(benzyloxy)-2-(methylsulfonyl)-5,8-dihydropyrido[3,4-d]pyrimidine-7(6H)-carboxylate (840 mg, 2.00 mmol), toluene (30 mL), ((2R,7aS)-2-fluorohexahydro-1H-pyrrolozin-7a-yl)methanol (383 mg, 2.40 mmol) and sodium tert-butoxide (289 mg, 3.00 mmol) was stirred at 0° C. under protection of nitrogen atmosphere for 1 hour and continued stirring at room temperature for 1 hour. The reaction mixture was purified by column chromatography (mobile phase: methanol/dichloromethane 1/30 to 1/10) to afford compound 51-g (561 mg, 56%) LC-MS (ESI): m/z 499.8(M+H)⁺.

### Synthesis of Compound 51-f

A reaction vial charged with 51-g (560 mg, 1.12 mmol), toluene (6 mL), trifluoroacetic acid (0.86 mL, 11.23 mmol) was stirred at room temperature under atmosphere nitrogen for 2 hours. The reaction mixture was added sodium hydroxide (2M, 10 mL), was adjusted pH to 9.5-10, and the aqueous phase was extracted with EA (10 mL*3). The organic phase was concentrated and the residue was purified by reversed-phase column under basic conditions to give compound 51-f (371 mg, 83%). LC-MS (ESI): m/z 399.7(M+H)⁺.

### Synthesis of Compound 51-e

To a reaction flask were added 51-f (500 mg, 1.25 mmol), ethanol (30 mL), Pd/C10% (100 mg, 10% content), and the reaction mixture was stirred at room temperature under hydrogen (1000 mL) for 3 hours. The reaction mixture was filtered, washed with ethanol (10 mL) and the filtrate was concentrated to give compound 51-e (260 mg, 67%). LC-MS (ESI): m/z 309.3 (M+H)⁺.

### Synthesis of Compound 51-d

A reaction vial charged with 51-e (180 mg, 0.58 mmol), toluene (15 mL), ((8-bromo-2-fluoro-6-(methoxymethoxy)naphthalen-1-yl)ethynyl)triisopropylsilane (331 mg, 0.72 mmol), sodium tert-butoxide (280 mg, 2.92 mmol), BINAP (73 mg, 0.12 mmol ) was stirred at room temperature under protection of nitrogen for 2 min, then was added Pd₂(dba)₃ (53 mg, 0.06 mmol), and the mixture continued stirring at 110° C. for 3 h under protection of nitrogen. The reaction mixture was added silica gel and purified directly by column chromatography (mobile phase: methanol/dichloromethane=1/30, 1/10) to afford compound 51-d (232 mg, 57%). LC-MS (ESI): m/z 693.6 (M+H)⁺.

### Synthesis of Compound 51-c

To a reaction flask, 51-d (90 mg, 0.13 mmol), DMF (15 mL), cesium fluoride (395 mg, 2.60 mmol) were added and the mixture was stirred at room temperature for 3 hours. The reaction mixture was filtered and washed with DMF (15 mL) to obtain a solution of 51-c in DMF which was directly used in the next step. LC-MS (ESI): m/z 537.5(M+H)⁺.

### Synthesis of Compound 51-b

To a reaction flask were added the solution of 51-c in DMF and Pd/C10% (12 mg, 10% content). The resulting was stirred at room temperature for 3 hours under hydrogen (1000 mL) atmosphere. The reaction mixture was filtered, quenched by adding water (50 mL) and the aqueous phase was extracted with ethyl acetate (30 mL*3). The organic phase was concentrated and the residue was purified by column chromatography (MeOH/DCM=1/100;1/10) to afford compound 51-b (57 mg, 81% overall yield in two steps). LC-MS (ESI): m/z 541.5(M+H)⁺.

### Synthesis of Compound 51-a

A reaction vial charged with 51-b (59 mg, 0.11 mmol), DMSO (5 mL), DIPEA (70 mg, 0.55 mmol) and N-bis(trifluoromethanesulfonyl)aniline (117 mg, 0.33 mmol) was stirred at room temperature for 2 hours. Then 1,2-diazepine dihydrochloride (50 mg, 0.29 mmol) and DIPEA (70 mg, 0.55 mmol) were added to DMSO (5 mL) and stirred for 30 min, and then this reaction mixture was added to the above mixture. The resulting mixture was stirred at 50° C. under atmosphere of nitrogen for 5 hours. The reaction mixture was concentrated and water (30 mL) was added. It was filtered and the filter cake was purified by column chromatography (MeOH/DCM=1/100; 1/10) to afford compound 51-a (50 mg, 74%), LC-MS (ESI): m/z 623.4(M+H)⁺.

### Synthesis of Compound 51

A reaction vial charged with 51-a (45 mg, 0.07 mmol), dichloromethane (5 mL) was stirred for 10 min in an ice-water bath, then was added 2,2-bipyridine (200 mg, 0.82 mmol) and TMSOTf (200 mg, 0.90 mmol). Formic acid (5 mL) was slowly added dropwise to the mixture and the reaction mixture was warmed to room temperature under protection of nitrogen and stirred for 12 hours. The reaction mixture was concentrated at room temperature, was added saturated sodium bicarbonate solution (30 mL) and the aqueous phase was extracted with dichloromethane (30 mL*2). The organic phase was concentrated and the residue was purified by column chromatography (MeOH/DCM=1/10) to give compound 51 (7.1 mg, 17%). LC-MS (ESI): m/z 579.5(M+H)⁺.

### Example 50 Synthetic Route of Compound 52

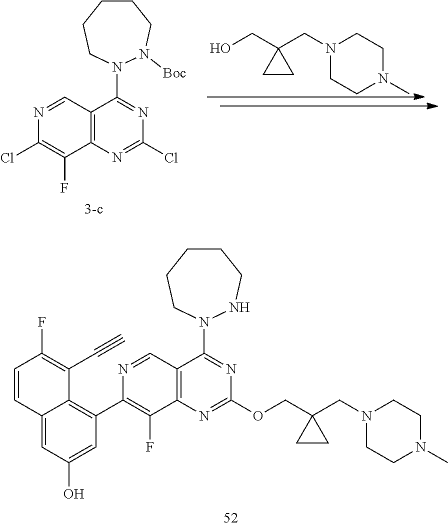 [SMILES: CC(C)(C)OC(=O)N1CCCCCN1c1nc(Cl)nc2c(F)c(Cl)ncc12 ; CN1CCN(CC2(CO)CC2)CC1 ; C#Cc1c(F)ccc2cc(O)cc(-c3ncc4c(N5CCCCCN5)nc(OCC5(CN6CCN(C)CC6)CC5)nc4c3F)c12]

### Synthesis of Compound 52

Referring to synthetic route of compound 29, compound 52 was synthesized using (1-((4-methylpiperazin-1-yl)methyl)cyclopropyl)methanol instead of (1-(morpholinomethyl)cyclopropyl)methanol and 3-c as material. LC-MS (ESI): m/z 614.3 (M+H)⁺.

### Example 51 Synthetic Route of Compound 53

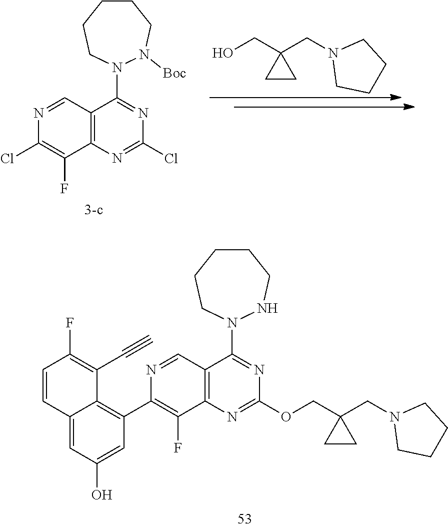 [SMILES: CC(C)(C)OC(=O)N1CCCCCN1c1nc(Cl)nc2c(F)c(Cl)ncc12 ; OCC1(CN2CCCC2)CC1 ; C#Cc1c(F)ccc2cc(O)cc(-c3ncc4c(N5CCCCCN5)nc(OCC5(CN6CCCC6)CC5)nc4c3F)c12]

### Synthesis of Compound 53

Referring to synthetic route of compound 29 replacement of (1-(morpholinomethyl)cyclopropyl)methanol with (1-(pyrrolidin-1-ylmethyl)cyclopropyl)methanol, and using 3-c as material to obtain compound 53. LC-MS (ESI): m/z 585.3 (M+H)⁺.

### Example 52 Synthetic Route of Compound 54

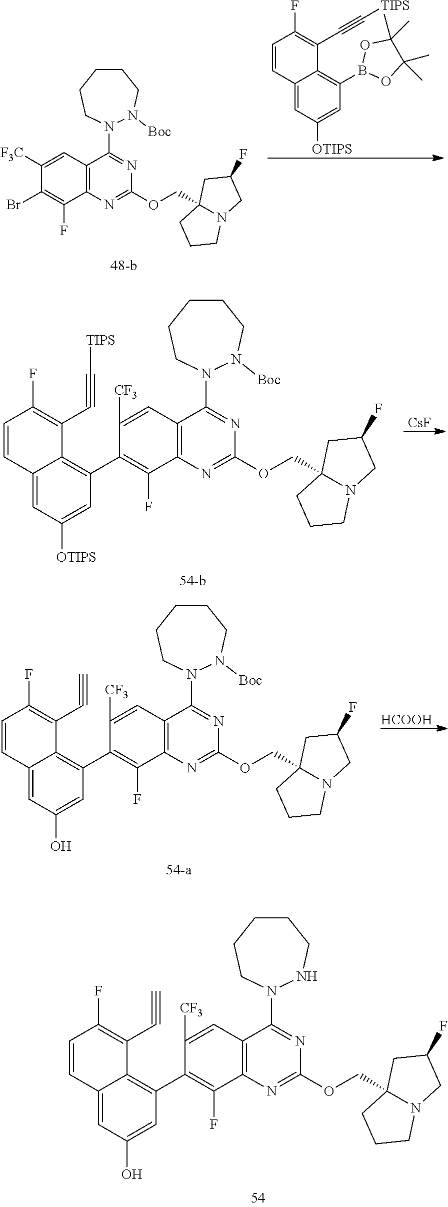

### Synthesis of Compound 54-b

To a microwave tube were added 48-b (160 mg, 0.25 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (461 mg, 0.74 mmol), Pd(DPEphos)Cl₂ (35 mg, 0.049 mmol), cesium carbonate (401 mg, 1.23 mmol), toluene (15 mL). After degassed and purged with nitrogen for three times, the microwave tube was sealed, microwaved at 150° C., and stirred for 6 hours. The reaction mixture was concentrated to dryness and the residue was purified by column chromatography (mobile phase: methanol: dichloromethane=0-5%) to give compound 54-b (60 mg, 23%). LC-MS (ESI): m/z 1068.5 (M+H)⁺.

### Synthesis of Compound 54-a

To a solution of 54-b (60 mg, 0.056 mmol) in N,N-dimethylformamide (10 mL) was added cesium fluoride (85 mg, 0.56 mmol) at room temperature and the mixture was stirred at room temperature overnight. The reaction mixture was poured a large amount of water and the aqueous phase was extracted with ethyl acetate (50 mL*2). The organic phase was washed with brine (30 mL*3), concentrated to dryness, and the residue was purified by column chromatography (mobile phase: dichloromethane: methanol: dichloromethane=0-7%) to give compound 54-a (15 mg, 35%). LC-MS (ESI): m/z 756.7 (M+H)⁺.

### Synthesis of Compound 54

A mixture of 54-a (15 mg, 0.020 mmol) and formic acid (6 mL) was stirred at room temperature overnight. The reaction mixture was concentrated to dryness at room temperature, purified by prep-HPLC (ammonium bicarbonate) and lyophilized to give compound 54 (3 mg, 23%). LC-MS (ESI): m/z 656.2 (M+H)⁺.

### Synthetic Route of Compound 55

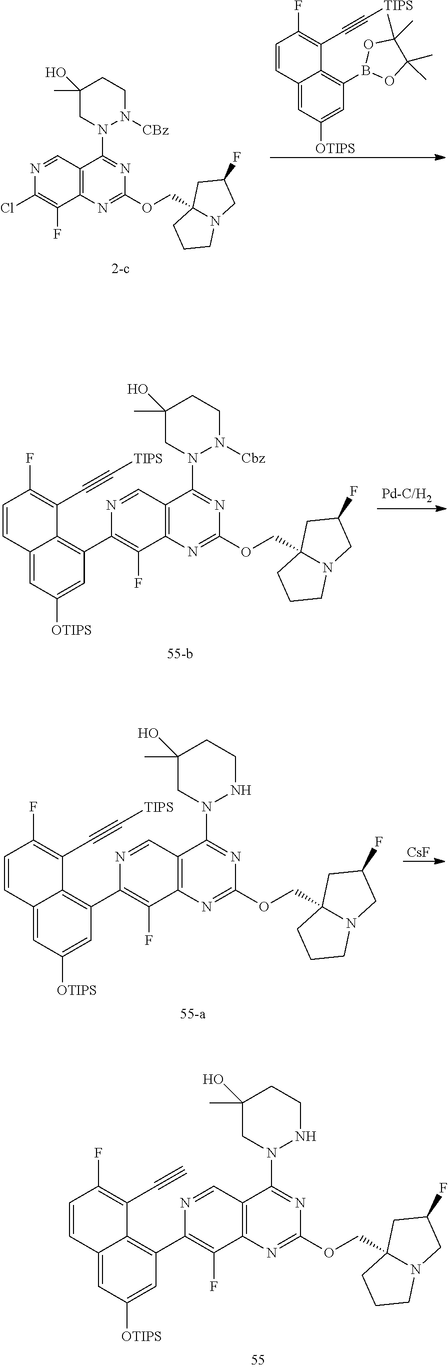

### Synthesis of Compound 55-b

To a sealed tube at room temperature was added 2-c (230 mg, 0.39 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (366 mg, 0.59 mmol), cataCXium A Pd G3 (28 mg, 0.04 mmol), potassium phosphate (249 mg, 1.17 mmol), THF (2 mL) and water (0.5 mL). After degassed and purged with nitrogen, the reaction mixture was stirred at 65° C. overnight. The reaction mixture was concentrated and the residue was purified by column chromatography (mobile phase: DCM/MeOH 10/0 to 10/1) to give compound 55-b (280 mg, 68%).

### Synthesis of Compound 55-a

At room temperature, to a reaction vial were added 55-b (280 mg, 0.27 mmol), EA (30 mL) and Pd/C (10%, 200 mg). After degassed and purged with hydrogen, the reaction mixture was stirred under hydrogen protection for 2 hours at room temperature. The reaction mixture was filtered and the filtrate was concentrated to give compound 55-a (190 mg, 78%). LC-MS (ESI): m/z 917.9 (M+H)⁺.

### Synthesis of Compound 55

55-a (190 mg, 0.21 mmol), DMF (3 mL) and cesium fluoride (315 mg, 2.07 mmol) were added to a reaction vial at room temperature. It was stirred at room temperature for 5 hours. The reaction mixture was filtered and the filtrate was purified by prep-HPLC to give compound 55 (55 mg, 44%).

LC-MS (ESI): m/z 605.8 (M+H)⁺.

### Synthesis of Compounds 55-1 and 55-2

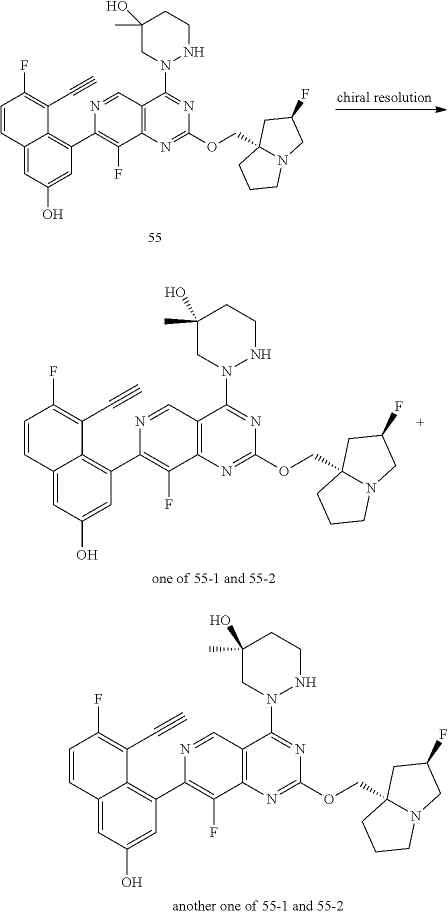 [SMILES: C#Cc1c(F)ccc2cc(O)cc(-c3ncc4c(N5CC(C)(O)CCN5)nc(OC[C@@]56CCCN5C[C@H](F)C6)nc4c3F)c12 ; C#Cc1c(F)ccc2cc(O)cc(-c3ncc4c(N5C[C@](C)(O)CCN5)nc(OC[C@@]56CCCN5C[C@H](F)C6)nc4c3F)c12 ; C#Cc1c(F)ccc2cc(O)cc(-c3ncc4c(N5C[C@@](C)(O)CCN5)nc(OC[C@@]56CCCN5C[C@H](F)C6)nc4c3F)c12]

compound 55 (55 mg) was separated by chiral resolution to obtain compound 55-1(15 mg, 27%) and 55-2 (19 mg, 35%)

| Conditions for chiral analysis | Conditions for chiral preparation |
|---|---|
| Instrument: UPCC (Waters) | Instrument: SFC-150 (Waters) |
| Column OX 4.6 × 100 mm, 3 um (Daicel) | Column OX 25*250 mm, 10 um (Daicel) |
| Column temperature: 40° C. | Column temperature: 35° C. |
| Mobile phase: CO₂/MeOH[0.2% NH₃(7M | Mobile phase: CO₂/MEOH[0.2% NH₃(7M in |
| in MeOH)] = 60/40 | MeOH)] = 50/50 |
| Flow rate: 3.0 mL/min | Flow rate: 100 mL/min |
| Wavelength: 214 nm | Pressure: 100 bar |
| Pressure: 2000 psi | Wavelength: 214 nm |
| | Circulation: 6.0 min |
| | Sample solution: 55 mg was dissolved in 30 mL |
| | of methanol |
| 55-1: the retention time was 1.745 min; | |
| e.e. % = 100.0%; | |
| 55-2: the retention time was 2.827 min; | |
| e.e. % = 99.3%. | |

### Synthetic Route of Compound 56

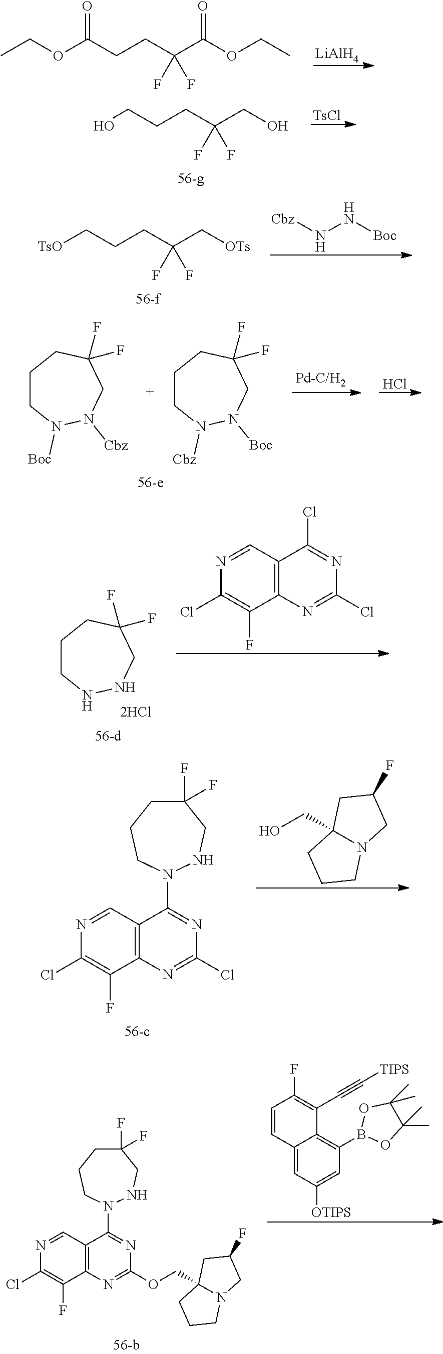

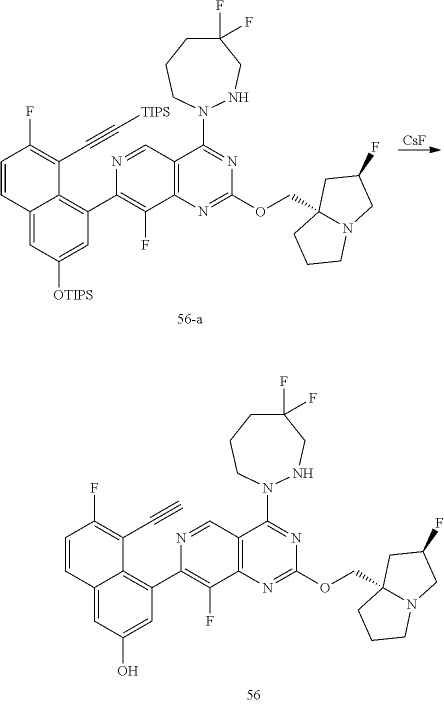

### Synthesis of Compound 56-g

Diethyl 2,2-difluoroglutarate (1000 mg, 4.46 mmol) and THF (10 mL) were added to a reaction flask at room temperature. After degassed and purged with nitrogen, the above mixture was cooled down to 0° C., and was added a solution of lithium aluminum hydride (1 M, 11 mL, 11.00 mmol) in tetrahydrofuran dropwise. After the dropwise addition, the reaction mixture was naturally warmed to room temperature and stirred overnight. The reaction mixture was quenched with methanol (100 mL), was added sodium sulfate decahydrate (5 g) and stirred for 1 hour. The reaction mixture was filtered through celite and the filtrate was concentrated. The residue was added ethyl acetate, a solid precipitated, filtered and the filtrate was concentrated to give compound 56-g (550 mg, 88%).

### Synthesis of Compound 56-f

At room temperature, to a reaction vial were added 56-g (550 mg, 3.93 mmol), DCM (30 mL), triethylamine (1589 mg, 15.70 mmol) and p-toluenesulfonyl chloride (2245 mg, 11.78 mmol). After degassed and purged with nitrogen, the reaction mixture was stirred at room temperature overnight. Water was added to the reaction mixture and the aqueous phase was extracted with DCM. The organic phases were combined, washed with water, brine, dried over anhydrous sodium sulfate, concentrated, and the residue was purified by column chromatography (mobile phase: PE/EA 10/0 to 6/4) to give compound 56-f (960 mg, 55%). LC-MS (ESI): m/z 466.7 (M+NH₄)⁺.

### Synthesis of Compound 56-e

At room temperature, to a reaction vial were added 56-f (500 mg, 1.12 mmol), acetonitrile (30 mL), 2-(tert-butyl)hydrazine-1,2-dicarboxylic acid-1-benzyl ester (297 mg, 1.12 mmol) and cesium carbonate (1816 mg, 5.57 mmol). After degassed and purged with nitrogen, the reaction mixture was stirred at 65° C. for 36 hours. The reaction mixture was filtered, and the filtrate was concentrated and the residue was purified by column chromatography (mobile phases: PE/EA 10/0 to 7/3) to give compound 56-e (230 mg, 56%). LC-MS (ESI): m/z 388.4 (M+NH₄)⁺.

### Synthesis of Compound 56-d

56-e (230 mg, 0.62 mmol), EA (25 mL) and Pd/C (10%, 100 mg) were added to a reaction vial at room temperature. The reaction mixture was degassed and purged with hydrogen for 4 hours at room temperature. The reaction mixture was filtered, and the filtrate was concentrated and the residue was added acetonitrile (10 mL) and HCl/1,4-dioxane (1M, 2 mL, 2.00 mmol). The reaction mixture was stirred at room temperature After degassed and purged with nitrogen for 2 hours, the reaction mixture was concentrated to give the crude compound 56-d (150 mg). LC-MS (ESI): m/z 137.2 (M+1)⁺.

### Synthesis of Compound 56-c

At room temperature, to a reaction vial were added 2,4,7-trichloro-8-fluoropyrido[4,3-d]pyrimidine (313 mg, 1.24 mmol), DCM (15 mL) and crude compound 56-d (150 mg, 0.62 mmol). The mixture was degassed and purged with nitrogen, cooled to 0° C., and was added DIPEA (481 mg, 3.72 mmol). The mixture was naturally warmed to room temperature and stirred overnight. Water was added to the reaction mixture and the aqueous phase was extracted with DCM. The organic phases were combined, dried, concentrated and the residue was purified by column chromatography (mobile phase: PE/EA 10/0 to 6/4) to give compound 56-c (110 mg, 50%). LC-MS (ESI): m/z 352.1 (M+H)⁺.

### Synthesis of Compound 56-b

At room temperature, to a reaction vial were added 56-c (110 mg, 0.31 mmol), ((2R,7aS)-2-fluorohexahydro-1H-pyrrolizin-7a-yl)methanol (99 mg, 0.62 mmol), 1,4-dioxane (5 mL) and DIPEA (162 mg, 1.25 mmol). After degassed and purged with nitrogen, the reaction mixture was stirred at 90° C. overnight. The reaction mixture was concentrated and the residue was purified by column chromatography (mobile phase: DCM/MeOH 10/0 to 10/1) to give compound 56-b (80 mg, 54%). LC-MS (ESI): m/z 475.9 (M+H)⁺.

### Synthesis of Compound 56-a

To a sealed tube at room temperature was added 56-b (80 mg, 0.17 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (158 mg, 0.25 mmol), cataCXium A Pd G3 (25 mg, 0.03 mmol), potassium phosphate (107 mg, 0.50 mmol), THF (2 mL) and water (0.5 mL). After degassed and purged with nitrogen, the reaction mixture was stirred at 65° C. overnight. The reaction mixture was concentrated and the residue was purified by column chromatography (mobile phase: DCM/MeOH 10/0 to 10/1) to give compound 56-a (150 mg, 95%).

### Synthesis of Compound 56

56-a (150 mg, 0.16 mmol), DMF (3 mL) and cesium fluoride (243 mg, 1.60 mmol) were added to a reaction vial at room temperature. The mixture was stirred at room temperature for 3 hours. Filtered, the filtrate was purified by reversed-phase column (mobile phase: water (0.01 M NH₄HCO₃)/acetonitrile 10/0 to 1/9) to give compound 56 (69 mg, 69%). LC-MS (ESI): m/z 625.4 (M+H)⁺.

### Synthetic Route of Comparative compound 2′

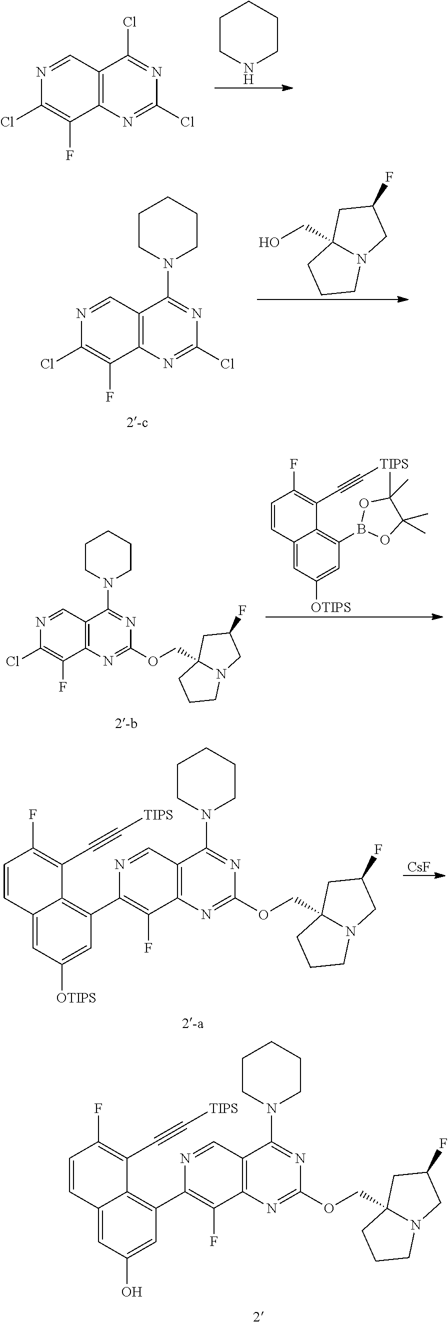

### Synthesis of Compound 2′-c

2,4,7-Trichloro-8-fluoropyrido[4,3-d]pyrimidine (330 mg, 1.31 mmol) and DCM (20 mL) were added to a reaction vial at room temperature. After degassed and purged with nitrogen, the above mixture was cooled to −78° C. in a dry ice-acetone bath and was added DIPEA (338 mg, 2.62 mmol) and piperidine (134 mg, 1.57 mmol). After addition, the mixture was naturally warmed to room temperature and stirred overnight. Water was added to the reaction mixture and the aqueous phase was extracted with DCM. The organic phases were combined, dried over anhydrous sodium sulfate, concentrated, and the residue was purified by column chromatography (mobile phase: PE/EA 10/0 to 6/4) to give compound 2′-c (320 mg, 81%).

### Synthesis of Compound 2′-b

At room temperature, to a reaction vial were added 2′-c (320 mg, 1.06 mmol), ((2R,7aS)-2-fluorohexahydro-1H-pyrrolizin-7a-yl)methanol (338 mg, 2.12 mmol), 1,4-dioxane (10 mL) and DIPEA (549 mg, 4.25 mmol). After degassed and purged with nitrogen, the reaction mixture was stirred at 90° C. overnight. The reaction mixture was concentrated and the residue was purified by column chromatography (mobile phase: DCM/MeOH 10/0 to 10/1) to give compound 2′-b (350 mg, 78%). LC-MS (ESI): m/z 424.4 (M+H)⁺.

### Synthesis of Compound 2′-a

To a sealed tube at room temperature was added 2′-b (50 mg, 0.12 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (111 mg, 0.18 mmol), cataCXium A Pd G3 (9 mg, 0.01 mmol), potassium phosphate (75 mg, 0.35 mmol), THF (2 mL) and water (0.5 mL). After degassed and purged with nitrogen, the reaction mixture was stirred at 65° C. overnight. The reaction mixture was concentrated and the residue was purified by column chromatography (mobile phase: DCM/MeOH 10/0 to 10/1) to afford compound 2′-a (67 mg, 64%).

### Synthesis of Compound 2′

2′-a (67 mg, 0.08 mmol), DMF (2 mL), and cesium fluoride (115 mg, 0.76 mmol) were added to a reaction vial at room temperature. After degassed and purged with nitrogen, the reaction mixture was stirred at 60° C. for 1 h. The reaction mixture was filtered and the filtrate was purified by prep-HPLC to give compound 2′ (29 mg, 67%). LC-MS (ESI): m/z 574.8 (M+H)⁺.

### Synthetic Route of Compound 57

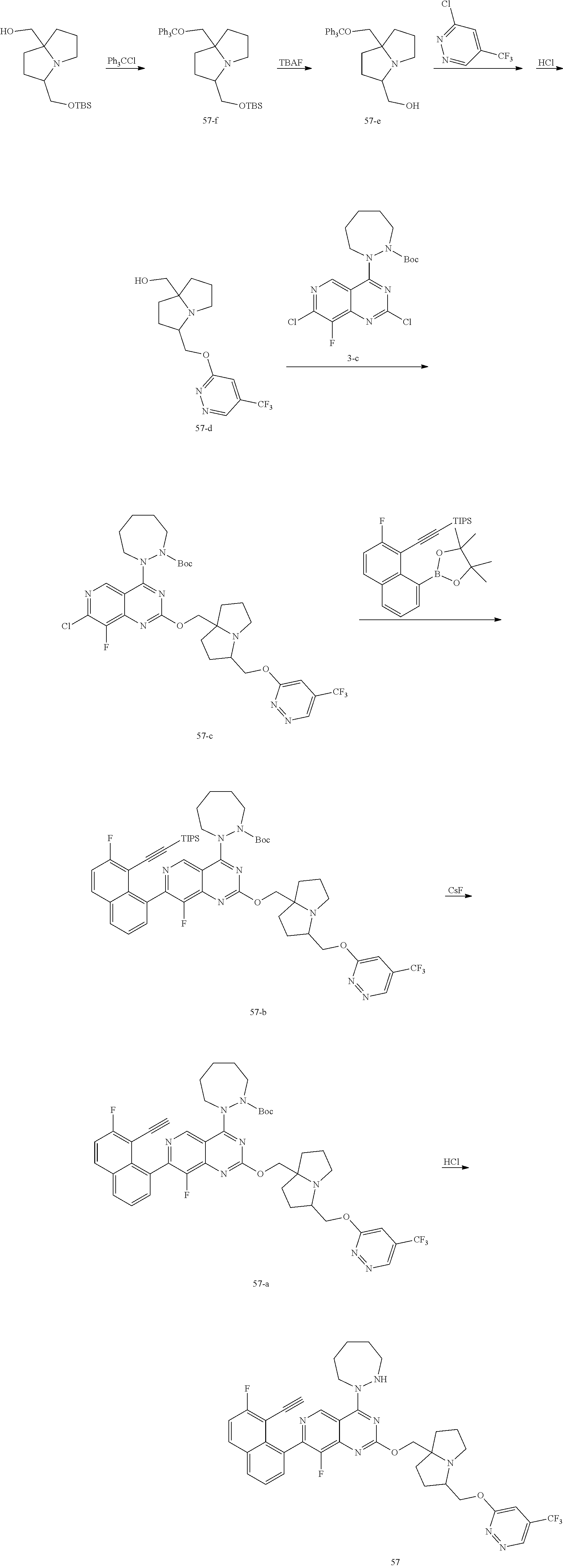

### Synthesis of Compound 57-f

Compound 3-[[tert-butyl(dimethyl)methylsilyl]oxymethyl]-1,2,3,5,6,7-hexahydropyrrolizin-7-yl]methanol (1.5 g, 0.0053 mol) was dissolved in 1,2-dichloroethane (10 mL), to which were added triethylamine (0.8 g, 0.0079 mol), 4-dimethylaminopyridine (60 mg, 0.49 mmol) and triphenylchloromethane (1.6 g, 0.0058 mol). After degassed and purged with nitrogen, the reaction mixture was stirred at 65° C. for 2 hours. The reaction mixture was added water (100 mL), extracted with ethyl acetate (100 mL). The organic phase was washed with brine (50 mL), dried over sodium sulfate, filtered off the desiccant, and the filtrate was concentrated at reduced pressure to obtain the crude product, which was purified by column chromatography (mobile phase, PE/EA 3/1) to obtain the compound 57-f (2.6 g, 94%). LC-MS (ESI): m/z 528.2 (M+H)⁺.

### Synthesis of Compound 57-e

Compound 57-f (2.6 g, 4.93 mmol) was dissolved in tetrahydrofuran (10 mL), to which was added a solution of tetrabutylammonium fluoride (1M, 19.7 mL, 19.7 mmol) in tetrahydrofuran, and the mixture was stirred for 2 h at 50° C. Water (100 mL) was added to the reaction mixture and the aqueous phase was extracted with ethyl acetate (150 mL). The organic phase was washed for four times with water (100 mL), brine (50 mL), dried over sodium sulfate, filtered off the desiccant, and the filtrate was concentrated at reduced pressure to obtain the crude product, which was purified by column chromatography (mobile phase, DCM/MeOH 10/1) to give compound 57-e (1.06 g. 52%).

LC-MS (ESI): m/z 414.5 (M+H)⁺.

### Synthesis of Compound 57-d

Compound 57-e (1.06 g, 2.56 mmol) was dissolved in tetrahydrofuran (5 mL), followed by addition of sodium tert-butanol (430 mg, 3.83 mmol) and the mixture was stirred for 30 min, then cooled in an ice-water bath, it was added 3-chloro-5-(trifluoromethyl)pyrazine (560 mg, 3.07 mmol), and the mixture was stirred for 1 h at room temperature. To the reaction mixture was added saturated ammonium chloride solution (1 mL) and water (50 mL) and the aqueous phase was extracted with ethyl acetate (50 mL). The organic phase was washed with brine (50 mL), dried over sodium sulfate, filtered off the desiccant, and the filtrate was concentrated at reduced pressure to obtain the crude product, which was dissolved in ethyl acetate (4 mL), to which was added hydrochloride/1,4-dioxane (4M, 1 mL), the reaction mixture was stirred for 1 hr at room temperature, and then the reaction mixture was concentrated at reduced pressure, and then neutralized by the addition of 7M ammonia methanol solution and concentrated to obtain the crude product, which was purified by column chromatography (mobile phase, DCM/MeOH 10/1) to obtain compound 57-d (520 mg, 64%). LC-MS (ESI): m/z 318.1 (M+H)⁺.

### Synthesis of Compound 57-c

Compound 3-c (614 mg, 1.47 mmol) and 57-d (520 mg, 1.64 mmol) were dissolved in dichloromethane (10 mL). In an ice-water bath, the mixture was added sodium tert-butanol (236 mg, 2.46 mmol) and stirred for 2 hr in an ice-water bath. To the reaction mixture saturated ammonium chloride solution was added to neutralize, water (100 mL) was added and the aqueous phase was extracted with dichloromethane (100 mL). The organic phase was washed with brine (100 mL), dried over sodium sulfate, filtered off the desiccant, and the filtrate was concentrated at reduced pressure to obtain the crude product, which was purified by column chromatography (mobile phase, DCM/MeOH 20/1) to give compound 57-c (565 mg, 55%). LC-MS (ESI): m/z 697.4 (M+H)⁺.

### Synthesis of Compound 57-b

In a microwave tube, compound 57-c (150 mg, 0.22 mmol) and 7-fluoro-8-[(triisopropylsilyl)ethynyl]naphthalene-1-boronic acid pinacol ester (102 mg, 0.23 mmol) were dissolved in tetrahydrofuran (4 mL) and water (1 mL), followed by addition of methanesulfonate [n-butylbis(1-adamantyl)phosphine] (2-amino-1,1′-biphenyl-2-yl)palladium(II) (16 mg, 0.021 mmol) and anhydrous potassium phosphate (183 mg, 0.86 mmol), and the reaction mixture was blown with nitrogen for 1 min and stirred for 12 h at 65° C. Water (50 mL) was added to the reaction mixture and the aqueous phase was extracted with ethyl acetate (50 mL). The organic phase was washed with brine (50 mL), dried over sodium sulfate, filtered off the desiccant, and the filtrate was concentrated at reduced pressure to obtain the crude product, which was purified by column chromatography (mobile phase, DCM/MeOH 20/1) to give compound 57-b (200 mg, 94%). LC-MS (ESI): m/z 988.0 (M+H)⁺.

### Synthesis of Compound 57-a

Compound 57-b (200 mg, 0.20 mmol) was dissolved in N,N-dimethylformamide (5 mL), to which cesium fluoride (308 mg, 2.03 mmol) was added, and the reaction mixture was stirred for 1 h at room temperature. Water (100 mL) was added to the reaction mixture and the aqueous phase was extracted with ethyl acetate (50 mL). The organic phase was washed with brine (50 mL), dried over sodium sulfate, filtered off the desiccant, and the filtrate was concentrated at reduced pressure to obtain the crude product, which was purified by column chromatography (mobile phase, DCM/MeOH 20/1) to give compound 57-a (125 mg, 74%). LC-MS (ESI): m/z 831.6 (M+H)⁺.

### Synthesis of Compound 57

Compound 57-a (120 mg, 0.14 mmol) was dissolved in acetonitrile (3 mL), to which hydrochloride/1,4-dioxane (4 M, 0.3 mL) was added, and the reaction mixture was stirred for 12 h at room temperature. The reaction mixture was neutralized by adding saturated sodium bicarbonate solution and the aqueous phase was extracted with ethyl acetate (50 mL). The organic phase was washed with brine (50 mL), dried over sodium sulfate, filtered off the desiccant, and the filtrate was concentrated at reduced pressure to obtain the crude product, which was purified by prep-HPLC (basic conditions) to give compound 57 (32 mg, 30%). LC-MS (ESI): m/z 731.0(M+H)⁺.

### Synthetic Route of Compound 58

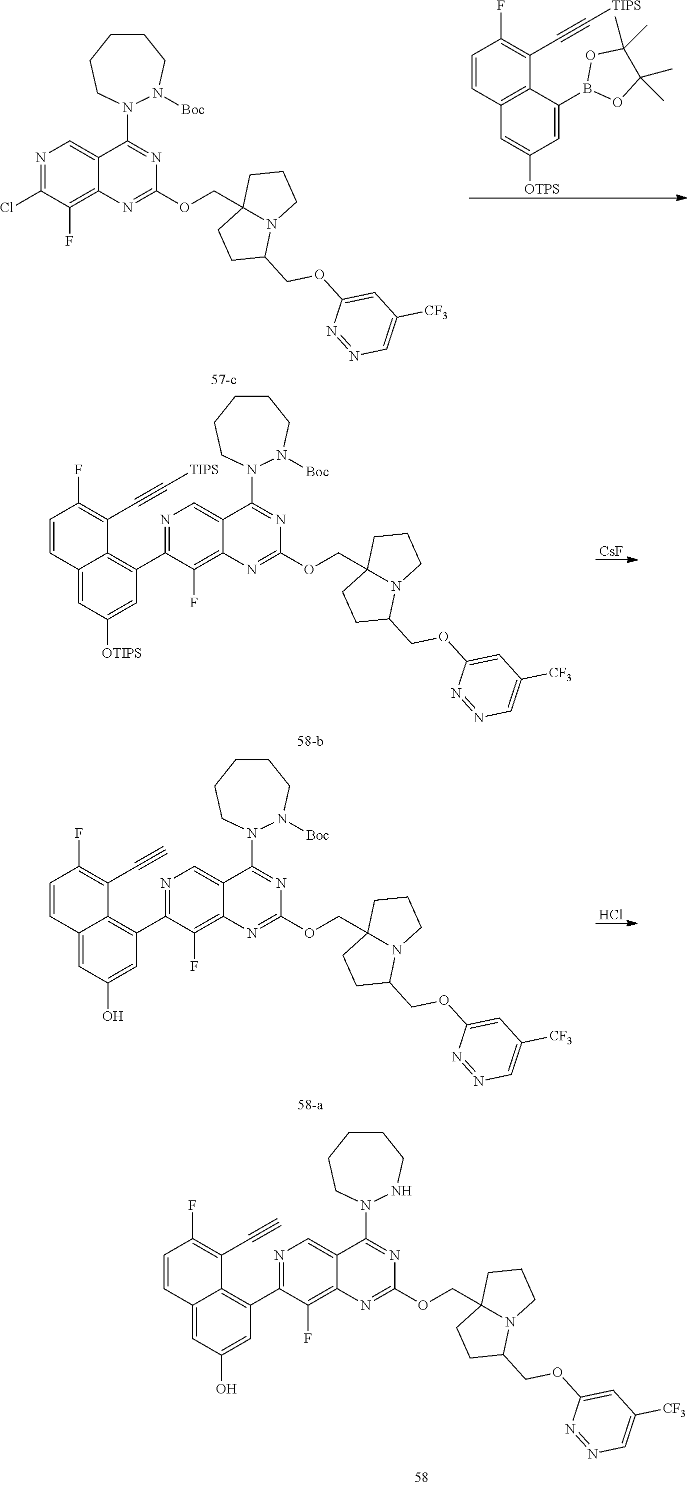

### Synthesis of Compound 58-b

In a microwave tube, compound 57-c (177 mg, 0.25 mmol) and 7-fluoro-8-[(triisopropylsilyl)ethynyl]-3-[(triisopropylsilyl)oxy]-1-naphthalenyl]boronic acid pinacol ester (175 mg, 0.28 mmol) were dissolved in tetrahydrofuran (4 mL) and water (1 mL), followed by addition of methanesulfonated[butylbis(1-adamantyl)phosphine] (2-amino-1,1′-biphenyl-2-yl)palladium(II) (37 mg, 0.051 mmol) and anhydrous potassium phosphate (216 mg, 1.02 mmol), and the reaction mixture was purged with nitrogen for 1 minute and stirred at 65° C. for 12 hours. Water (50 mL) was added to the reaction mixture and the aqueous phase was extracted with ethyl acetate (50 mL). The organic phase was washed with brine (50 mL), dried over sodium sulfate, filtered off the desiccant, and the filtrate was concentrated at reduced pressure to obtain the crude product, which was purified by column chromatography (mobile phase, DCM/MeOH 20/1) to give compound 58-b (260 mg, 88%).

### Synthesis of Compound 58-a

Compound 58-b (260 mg, 0.22 mmol) was dissolved in N,N-dimethylformamide (5 mL), to which cesium fluoride (341 mg, 2.24 mmol) was added, and the reaction mixture was stirred at room temperature for 4 hours. Water (100 mL) was added to the reaction mixture and the aqueous phase was extracted with ethyl acetate (100 mL). The organic phase was washed with brine (100 mL), dried over sodium sulfate, filtered off the desiccant, and the filtrate was concentrated at reduced pressure to obtain the crude product, which was purified by column chromatography (mobile phase, DCM/MeOH 20/1) to give compound 58-a (150 mg, 79%). LC-MS (ESI): m/z 847.2 (M+H)⁺.

### Synthesis of Compound 58

Compound 58-a (150 mg, 0.18 mmol) was dissolved in acetonitrile (3 mL), to which hydrochloride/1,4-dioxane (4 M, 0.3 mL) was added, and the reaction mixture was stirred for 12 h at room temperature. The reaction mixture was neutralized by adding saturated sodium bicarbonate solution and the aqueous phase was extracted with ethyl acetate (50 mL). The organic phase was washed with brine (50 mL), dried over sodium sulfate, filtered off the desiccant, and the filtrate was concentrated at reduced pressure to obtain the crude product, which was purified by prep-HPLC (basic conditions) to give compound 58 (50 mg, 38%). LC-MS (ESI): m/z 747.6(M+H)+¹H NMR (400 MHz, DMSO-d₆): δ 10.23 (1H, s), 10.13 (1H, s), 9.30 (1H, s), 8.00-7.94 (1H, m), 7.74 (1H, s), 7.49-7.42 (1H, m), 7.38 (1H, s), 7.19-7.16 (1H, m), 5.88-5.83 (1H, m), 4.48-4.35 (2H, m), 4.16-4.90 (4H, m), 3.94-3.90 (1H, m), 3.21-3.12 (1H, m), 3.08-2.95 (3H, m), 2.81-2.73 (1H, m), 2.15-2.02 (2H, m), 1.95-1.60 (12H, m).

### Synthetic Route of Compound 59

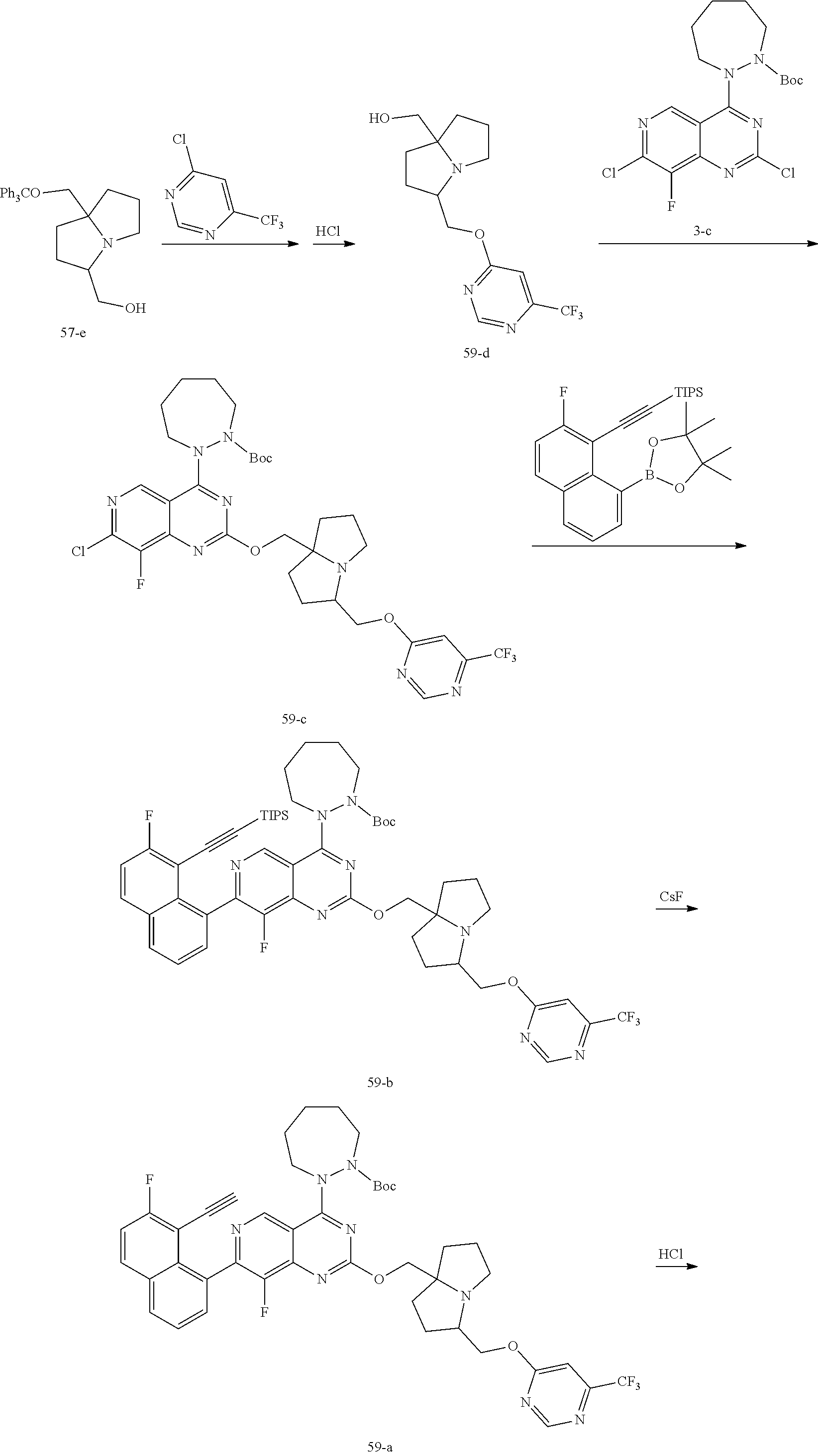

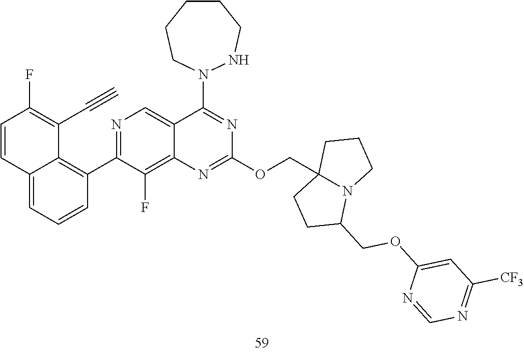 [SMILES: C#Cc1c(F)ccc2cccc(-c3ncc4c(N5CCCCCN5)nc(OCC56CCCN5C(COc5cc(C(F)(F)F)ncn5)CC6)nc4c3F)c12]

### Synthesis of Compound 59-d

Compound 57-e (310 mg, 0.75 mmol) was dissolved in tetrahydrofuran (10 mL), to which potassium tert-butanolate (126 mg, 1.12 mmol) was added and the resulting mixture was stirred for 30 min.

Then placed in an ice-water bath, the mixture was added 4-chloro-6-trifluoromethylpyrimidine (164 mg, 0.90 mmol), and was stirred for 2 h at room temperature. Water (50 mL) was added to the reaction mixture and the aqueous phase was extracted with ethyl acetate (50 mL). The organic phase was washed with brine (50 mL), dried over sodium sulfate, filtered off the desiccant and the filtrate was concentrated at reduced pressure to obtain the crude product. The crude product was dissolved in ethyl acetate (2 mL), to which hydrochloride/1,4-dioxane (4M, 0.5 mL) was added, and the reaction mixture was stirred at room temperature for 2 hours. The reaction mixture was concentrated at reduced pressure, to which 7M ammonia methanol solution was added to neutralize and concentrate to obtain the crude product, which was purified by column chromatography (mobile phase, DCM/MeOH 10/1) to obtain the compound 59-d (228 mg, 96%). LC-MS (ESI): m/z 318.3 (M+H)⁺.

### Synthesis of Compound 59-c

Compounds 3-c (299 mg, 0.72 mmol) and 59-d (228 mg, 0.72 mmol) were dissolved in dichloromethane (10 mL). In an ice-water bath, the above mixture was added sodium tert-butoxide (173 mg, 1.80 mmol) and the reaction mixture was stirred for 3 hr in an ice-water bath bath. The reaction mixture was neutralized by adding saturated ammonium chloride solution to the reaction mixture, water (100 mL) was added and the aqueous phase was extracted with dichloromethane (50 mL). The organic phase was washed with brine (50 mL), dried over sodium sulfate, filtered off the desiccant, and the filtrate was concentrated at reduced pressure to obtain the crude product, which was purified by column chromatography (mobile phase, DCM/MeOH 20/1) to obtain the compound 59-c (142 mg, 28%). LC-MS (ESI): m/z 697.4 (M+H)⁺.

### Synthesis of Compound 59-b

In a microwave tube, compound 59-c (140 mg, 0.20 mmol) and 7-fluoro-8-[(triisopropylsilyl)ethynyl]naphthalene-1-boronic acid pinacol ester (136 mg, 0.30 mmol) were dissolved in tetrahydrofuran (4 mL) and water (1 mL), followed by addition of methanesulfonated [n-butylbis(1-adamantyl)phosphine] (2-amino-1,1′-biphenyl-2-yl)palladium(II) (29 mg, 0.040 mmol), anhydrous potassium phosphate (171 mg, 0.81 mmol), and the reaction mixture was blown with nitrogen for 1 min and stirred for 12 h at 65° C. Water (50 mL) was added to the reaction mixture and the aqueous phase was extracted with ethyl acetate (50 mL). The organic phase was washed with brine (50 mL), dried over sodium sulfate, filtered off the desiccant, and the filtrate was concentrated at reduced pressure to obtain the crude product, which was purified by column chromatography (mobile phase, DCM/MeOH 20/1) to give compound the compound 59-b (110 mg, 56%). LC-MS (ESI): m/z 988.1(M+H)⁺.

### Synthesis of Compound 59-a

Compound 59-b (140 mg, 0.14 mmol) was dissolved in N,N-dimethylformamide (5 mL), to which cesium fluoride (215 mg, 1.42 mmol) was added, and the reaction mixture was stirred for 2 h at room temperature. Water (100 mL) was added to the reaction mixture and the aqueous phase was extracted with ethyl acetate (50 mL). The organic phase was washed with brine (50 mL), dried over sodium sulfate, filtered off the desiccant, and the filtrate was concentrated at reduced pressure to obtain the crude product, which was purified by column chromatography (mobile phase, DCM/MeOH 20/1) to give compound 59-a (100 mg, 85%). LC-MS (ESI): m/z 831.8 (M+H)⁺.

### Synthesis of Compound 59

Compound 59-a (100 mg, 0.12 mmol) was dissolved in acetonitrile (2 mL), to which hydrochloride/1,4-dioxane (4 M, 0.2 mL) was added, and the reaction mixture was stirred for 12 h at room temperature. The reaction mixture was neutralized with saturated sodium bicarbonate solution and the aqueous phase was extracted with ethyl acetate (50 mL). The organic phase was washed with brine (50 mL), dried over sodium sulfate, filtered off the desiccant, and the filtrate was concentrated at reduced pressure to obtain the crude product, which was purified by prep-HPLC (basic conditions) to give compound 59 (50 mg, 57%). LC-MS (ESI): m/z 731.0(M+H)⁺.

### Synthetic Route of Compound 60

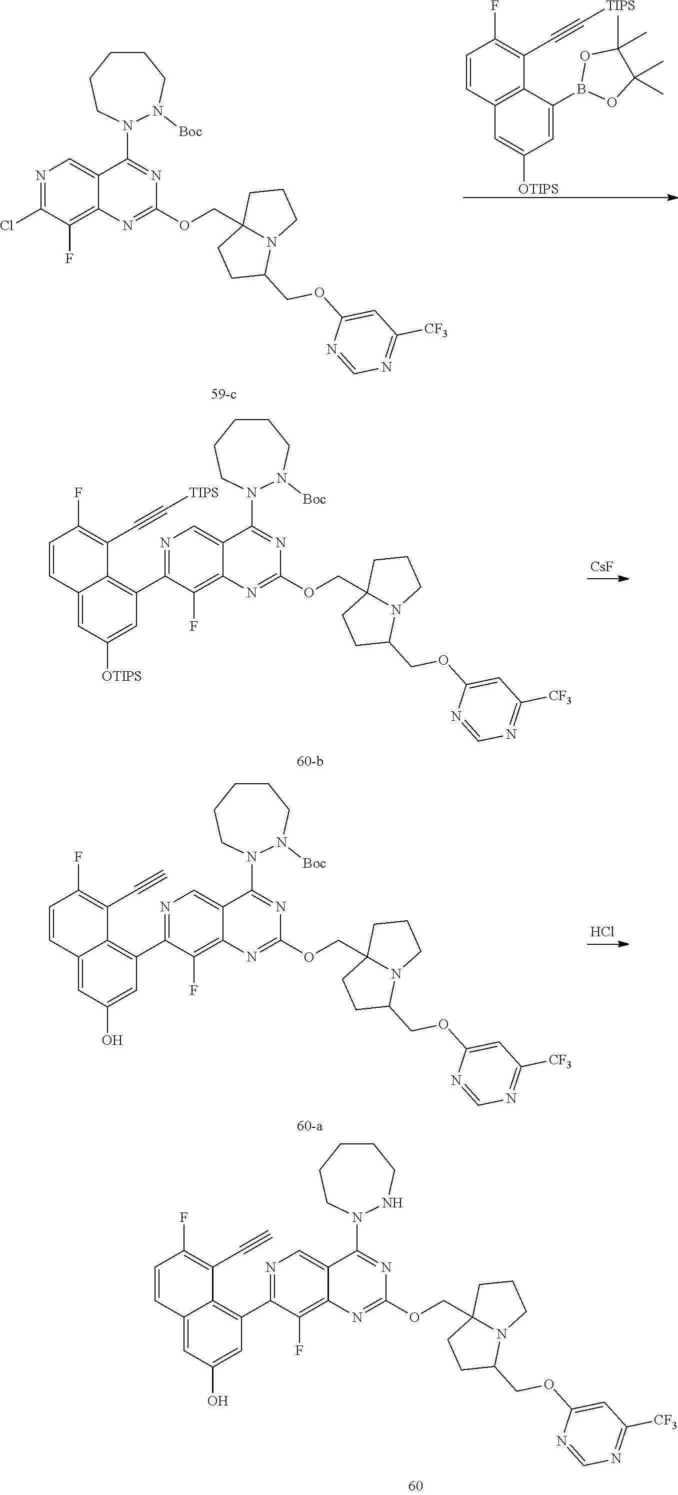

### Synthesis of Compound 60-b

In a microwave tube, compound 59-c (190 mg, 0.27 mmol) and 7-fluoro-8-[(triisopropylsilyl)ethynyl]-3-[(triisopropylsilyl)oxy]-1-naphthalenyl]boronic acid pinacol ester (255 mg, 0.41 mmol) were dissolved in tetrahydrofuran (4 mL) and water (1 mL), followed by addition of methanesulfonated [butylbis(1-adamantyl)phosphine] (2-amino-1,1′-biphenyl-2-yl)palladium(II) (40 mg, 0.055 mmol), and anhydrous potassium phosphate (231 mg, 1.09 mmol), and the reaction mixture was purged with nitrogen for 1 min, and stirred for 12 hr at 65° C. Water (50 mL) was added to the reaction mixture and the aqueous phase was extracted with ethyl acetate (50 mL). The organic phase was washed with brine (50 mL), dried over sodium sulfate, filtered off the desiccant, and the filtrate was concentrated at reduced pressure to obtain the crude product, which was purified by column chromatography (mobile phase, DCM/MeOH 20/1) to give compound 60-b (200 mg, 63%). LC-MS (ESI): m/z 1159.9 (M+H)⁺.

### Synthesis of Compound 60-a

Compound 60-b (200 mg, 0.17 mmol) was dissolved in N,N-dimethylformamide (5 mL), to which cesium fluoride (362 mg, 1.72 mmol) was added, and the reaction mixture was stirred at room temperature for 4 hours. Water (100 mL) was added to the reaction mixture and the aqueous phase was extracted with ethyl acetate (100 mL). The organic phase was washed with brine (100 mL), dried over sodium sulfate, filtered off the desiccant, and the filtrate was concentrated at reduced pressure to obtain the crude product, which was purified by column chromatography (mobile phase, DCM/MeOH 20/1) to give compound 60-a (120 mg, 82%). LC-MS (ESI): m/z 847.7 (M+H)⁺.

### Synthesis of Compound 60

Compound 60-a (120 mg, 0.14 mmol) was dissolved in acetonitrile (4 mL), to which hydrochloride/1,4-dioxane (4 M, 0.4 mL) was added, and the reaction mixture was stirred for 12 h at room temperature. The reaction mixture was neutralized with saturated sodium bicarbonate solution and the aqueous phase was extracted with ethyl acetate (50 mL). The organic phase was washed with brine (50 mL), dried over sodium sulfate, filtered off the desiccant, and the filtrate was concentrated at reduced pressure to obtain the crude product, which was purified by prep-HPLC (basic conditions) to give compound 60 (50 mg, 47%). LC-MS (ESI): m/z 747.3(M+H)⁺.

### Synthetic Route of Compound 61

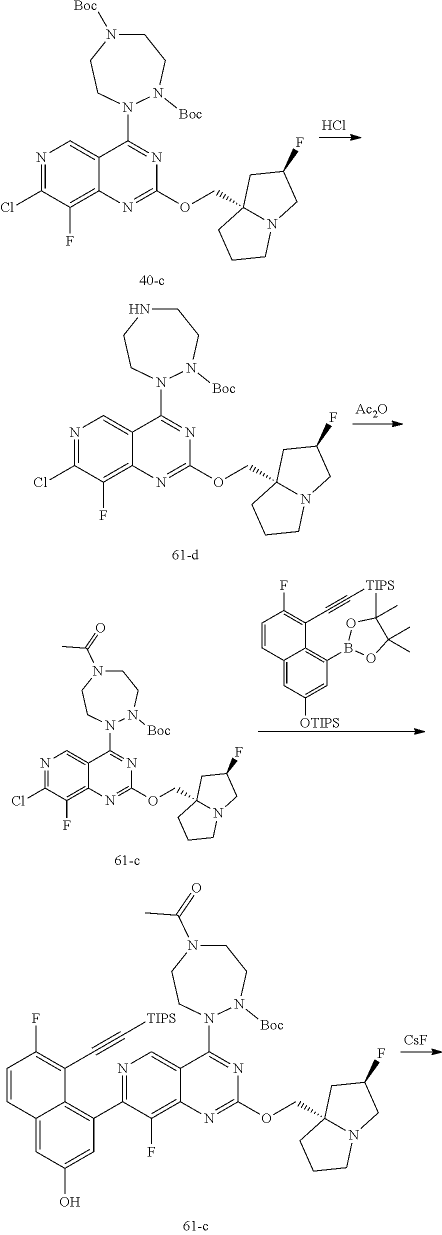

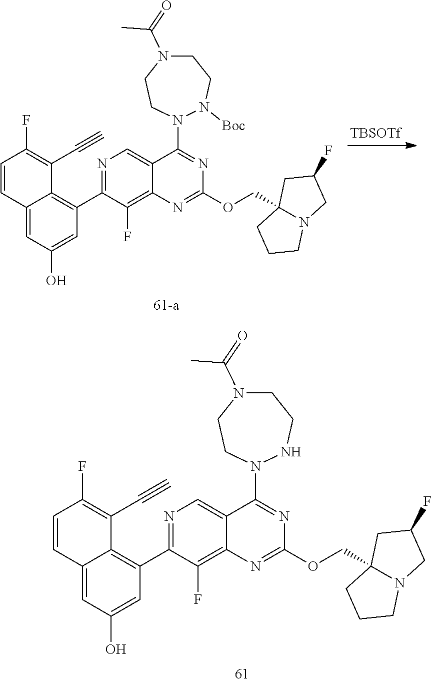 [SMILES: "TBSOTf" ; C#Cc1c(F)ccc2cc(O)cc(-c3ncc4c(N5CCN(C(C)=O)CCN5C(=O)OC(C)(C)C)nc(OC[C@@]56CCCN5C[C@H](F)C6)nc4c3F)c12 ; C#Cc1c(F)ccc2cc(O)cc(-c3ncc4c(N5CCN(C(C)=O)CCN5)nc(OC[C@@]56CCCN5C[C@H](F)C6)nc4c3F)c12]

### Synthesis of Compound 61-d

Compound 40-c (200 mg, 0.31 mmol) was dissolved in acetonitrile (10 mL), to which hydrochloride/1,4-dioxane (4 M, 1 mL) was added in an ice-water bath, and the reaction mixture was stirred in an ice-water bath for 2 hours. Saturated sodium bicarbonate solution was added to the reaction mixture to neutralize, water (10 mL) and the aqueous phase was extracted with ethyl acetate (50 mL). The organic phase was washed with brine (50 mL), dried over sodium sulfate, filtered off the desiccant, and the filtrate was concentrated at reduced pressure to obtain the crude product, which was purified by column chromatography (mobile phase, DCM/7M ammonia-methanol 10/1) to give compound 61-d (110 mg, 65%). LC-MS (ESI): m/z 540.3 (M+H)⁺.

### Synthesis of Compound 61-c

Compound 61-d (110 mg, 0.21 mmol) and acetic anhydride (31 mg, 0.30 mmol) were dissolved in dichloromethane (3 mL), followed by addition of 4-dimethylaminopyridine (37 mg, 0.30 mmol), and the mixture was stirred for 1 hour at room temperature. Water (50 mL) was added to it and the aqueous phase was extracted with dichloromethane (50 mL). The organic phase was washed with brine (50 mL), dried over sodium sulfate, filtered off the desiccant, and the filtrate was concentrated at reduced pressure to obtain the crude product, which was purified by column chromatography (mobile phase, DCM/MeOH 20/1) to give compound 61-c (110 mg, 93%). LC-MS (ESI): m/z 582.6 (M+H)⁺.

### Synthesis of Compound 61-b

In a microwave tube, compound 61-c (110 mg, 0.19 mmol) and pinacol ester of 7-fluoro-8-[(triisopropylsilyl)ethynyl]-3-[(triisopropylsilyl)oxy]-1-naphthalenyl]boronic acid (130 mg, 0.21 mmol) were dissolved in tetrahydrofuran (4 mL) with water (1 mL), followed by addition of methanesulfonated [butylbis(1-adamantyl) alkyl)phosphine](2-amino-1,1 ′-biphenyl-2-yl)palladium(II) (28 mg, 0.038 mmol) and anhydrous potassium phosphate (160 mg, 0.75 mmol), and the reaction mixture was purged with nitrogen for 1 min, and stirred for 12 h at 65° C. The reaction mixture was added water (50 mL), extracted with ethyl acetate (50 mL), washed with brine (50 mL), dried over sodium sulfate, filtered off the desiccant, and the filtrate was concentrated at reduced pressure to obtain the crude product, which was purified by column chromatography (mobile phase, DCM/MeOH 20/1) to obtain compound 61-b (130 mg, 77%). LC-MS (ESI): m/z 888.8(M+H)⁺.

### Synthesis of Compound 61-a

Compound 61-b (130 mg, 0.15 mmol) was dissolved in N,N-dimethylformamide (5 mL), to which cesium fluoride (222 mg, 1.46 mmol) was added, and the reaction mixture was stirred for 4 h at room temperature. Water (100 mL) was added to the reaction mixture and the aqueous phase was extracted with ethyl acetate (50 mL). The organic phase was washed with brine (50 mL), dried over sodium sulfate, filtered off the desiccant, and the filtrate was concentrated at reduced pressure to obtain the crude product, which was purified by column chromatography (mobile phase, DCM/MeOH 20/1) to obtain compound 61-a (90 mg, 84%). LC-MS (ESI): m/z 732.3 (M+H)⁺.

### Synthesis of Compound 61

Compound 61-a (90 mg, 0.12 mmol) was dissolved in dichloromethane (2 mL), to which tert-butyldimethylsilyl trifluoromethanesulfonate (33 mg, 0.12 mmol) was added, and the reaction mixture was stirred for 5 h at room temperature. Saturated sodium bicarbonate solution (10 mL) was added to the reaction mixture and continued stirring for 1 h. The reaction mixture was extracted with dichloromethane (50 mL). The organic phase was washed with brine (50 mL), dried over sodium sulfate, filtered off the desiccant, and the filtrate was concentrated at reduced pressure to obtain the crude product, which was purified by prep-HPLC (basic conditions) to give compound 61 (12 mg, 16%). LC-MS (ESI): m/z 632.3 (M+H)⁺.

### Synthetic Route of Compound 62

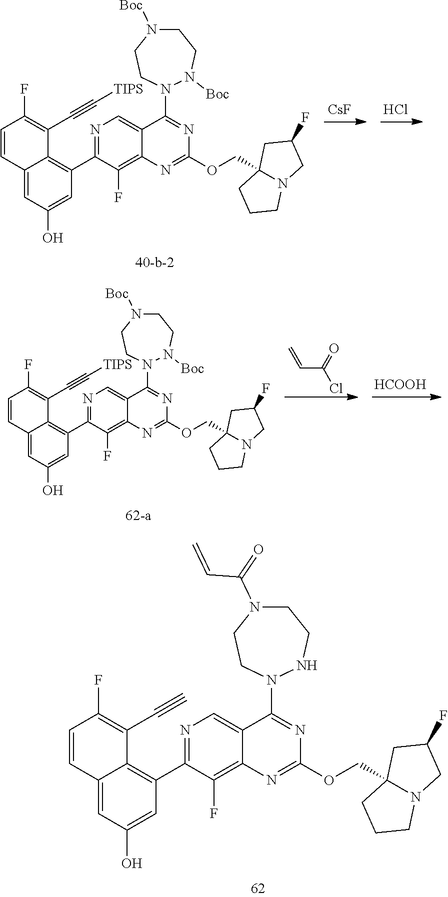

### Synthesis of Compound 62-a

Compound 40-b-2 (140 mg, 0.15 mmol) was dissolved in N,N-dimethylformamide (4 mL), to which cesium fluoride (225 mg, 1.48 mmol) was added, and the reaction mixture was stirred at room temperature for 4 hours. Water (100 mL) was added to the reaction mixture and the aqueous phase was extracted with ethyl acetate (100 mL). The organic phase was washed with brine (100 mL), dried over sodium sulfate, filtered off the desiccant, and the filtrate was concentrated at reduced pressure to obtain the crude product, which was purified by column chromatography (mobile phase, DCM/MeOH 20/1) to obtain the intermediate product, which was dissolved in acetonitrile (3 mL), followed by addition of hydrochloride/1,4-dioxane (4M, 0.3 mL) in an ice-water bath, then stirred for 2 h. The reaction mixture was neutralized with saturated sodium bicarbonate solution and the aqueous phase was extracted with ethyl acetate (100 mL). The organic phase was washed with brine (100 mL), dried over sodium sulfate, filtered off the desiccant, and the filtrate was concentrated at reduced pressure to give compound 62-a (80 mg, 78%). LC-MS (ESI): m/z 690.5 (M+H)⁺.

### Synthesis of Compound 62

Compound 62-a (80 mg, 0.12 mmol) was dissolved in dichloromethane (4 mL), followed by addition of diisopropylethylamine (30 mg, 0.23 mmol) and acryloyl chloride (13 mg, 0.14 mmol) and the reaction mixture was stirred for 1 h at room temperature. Water (50 mL) was added to the reaction mixture and the aqueous phase was extracted with dichloromethane (50 mL). The organic phase was washed with brine (50 mL), dried over sodium sulfate, filtered off the desiccant, and the filtrate was concentrated at reduced pressure to obtain the crude product, which was purified by column chromatography (mobile phase, DCM/MeOH 20/1) to obtain the intermediate product, which was dissolved in formic acid (2 mL), and the reaction mixture was stirred for 36 hours at room temperature. The reaction mixture was concentrated at reduced pressure to obtain the crude product, which was purified by prep-HPLC (basic conditions) to give compound 62 (17 mg, 23%).

LC-MS (ESI): m/z 644.3 (M+H)⁺.

### Synthetic Route of Compound 63

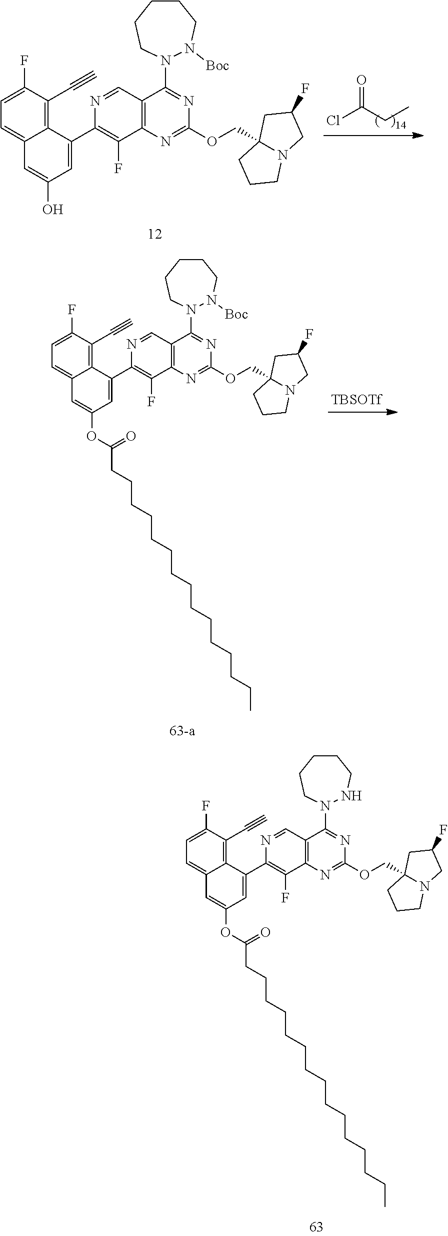

### Synthesis of Compound 63-a

Compound 12 (100 mg, 0.15 mmol) was dissolved in 20 mL of dichloromethane at room temperature, TEA (40 uL, 0.29 mmol) was added, palmitoyl chloride (120 mg, 0.44 mmol) was added to the reaction mixture in an ice-water bath, and the reaction mixture was warmed to room temperature and stirred for 1 hour. The reaction mixture was added 1 mL of methanol and stirred for 20 minutes. The reaction mixture was concentrated at reduced pressure and the residue was purified by a flash column chromatography (DCM/MeOH=20:1) to give the crude compound 63-a (134 mg). LC-MS (ESI): m/z=927.3 [M+H]⁺.

### Synthesis of Compound 63

Compound 63-a (134 mg, 0.14 mmol) was dissolved in 5 mL of DCM at room temperature and tert-butyldimethylsilyl trifluoromethanesulfonate (270 uL, 1.18 mmol) was added to the reaction mixture in an ice-water bath and the reaction mixture was stirred for 1 hour in an ice-water bath. The reaction mixture was added 10% sodium bisulfate solution (10 mL) at room temperature and stirred for 30 min, then saturated sodium bicarbonate solution was added and the pH was adjusted to basic, extracted with DCM (50 mL*2). The organic phase was dried over anhydrous sodium sulfate, filtered, and the filtrate was concentrated at reduced pressure, and the residue was purified by a flash column chromatography (DCM/MeOH=10:1) to give compound 63 (50 mg, 43%). LC-MS (ESI): m/z=827.4 [M+H]⁺.

### Synthetic Route of Compound 64

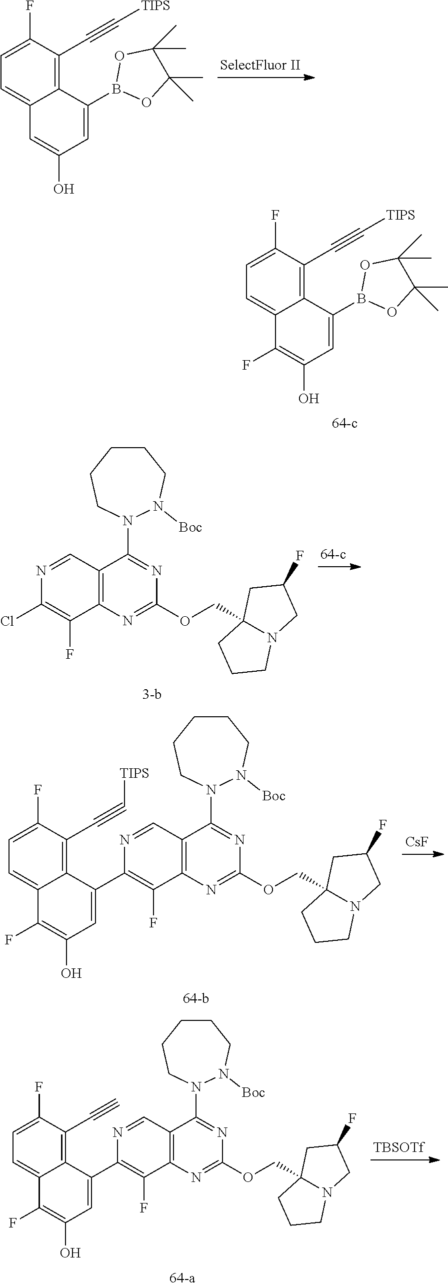

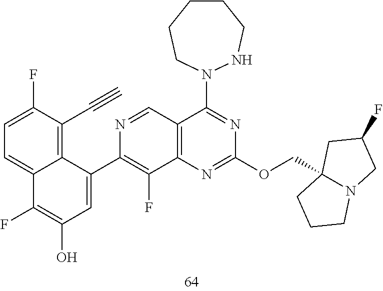 [SMILES: C#Cc1c(F)ccc2c(F)c(O)cc(-c3ncc4c(N5CCCCCN5)nc(OC[C@@]56CCCN5C[C@H](F)C6)nc4c3F)c12]

### Synthesis of Compound 64-c

At room temperature, compound 6-fluoro-4-(4,4,5,5-tetramethyl-1,3,2-dioxaborolan-2-yl)-5-((triisopropylsilyl)ethynyl)naphthalene-2-phenol (400 mg, 0.85 mmol) was dissolved in 20 mL of anhydrous acetonitrile, followed by addition of SelectFluor II (1-fluoro-4-methyl-1,4-diaza bicyclo[2.2.2]octane tetrafluoroborate) (410 mg, 1.28 mmol), and the reaction mixture was stirred at 80° C. under protection of nitrogen for 2 hours. The reaction mixture was cooled to room temperature, concentrated at reduced pressure and the residue was purified by flash column chromatography (PE/EA=3:1) to afford compound 64-c (145 mg, 35%). LC-MS (ESI): m/z=487.3 [M+H]⁺.

### Synthesis of Compound 64-b

At room temperature, 3-b (80 mg, 0.15 mmol), 64-c (79 mg, 0.16 mmol), methylsulfonyloxy(diadamantyl-n-butylphosphino)-2′-amino-1,1′-biphenyl-2-yl)palladium(II) (22 mg, 0.03 mmol), potassium phosphate (95 mg, 0.45 mmol), THF (2.5 mL), and water (0.5 mL) were combined in a microwave tube. After degassed and purged with nitrogen 3 times, the reaction mixture was stirred at 60° C. for 18 hours. The reaction mixture was cooled to room temperature, concentrated at reduced pressure, and the residue was purified by flash column chromatography (DCM/MeOH=20:1) to afford compound 64-b (66 mg, 51%). LC-MS (ESI): m/z=863.4 [M+H]⁺.

### Synthesis of Compound 64-a

Compound 64-b (66 mg, 0.076 mmol) was dissolved in 3 mL of DMF at room temperature, cesium fluoride (209 mg, 1.38 mmol) was added, and the mixture was stirred at room temperature under protection of nitrogen for 2 hours. The reaction mixture was added 20 mL of water, and the aqueous phase was extracted with ethyl acetate (50 mL*2). The organic phase was washed with brine (100 mL*5), dried over anhydrous sodium sulfate, filtered, and the filtrate was concentrated at reduced pressure to give the crude compound 64-a (47 mg, 87%). LC-MS (ESI): m/z=707.3 [M+H]⁺.

### Synthesis of Compound 64

Compound 64-a (47 mg, 0.067 mmol) was dissolved in 6 mL of dichloromethane at room temperature, tert-butyldimethylsilyl trifluoromethanesulfonate (200 uL, 0.87 mmol) was added in an ice-water bath, and the reaction mixture was stirred in an ice-water bath for 30 min. The reaction mixture was added 50 mL of saturated sodium bicarbonate solution, diluted with 30 mL of dichloromethane, and stirred at room temperature for 6 hours. The reaction mixture was extracted with dichloromethane (100 mL). The organic phase was dried over anhydrous sodium sulfate, filtered, and the filtrate was concentrated at reduced pressure to give compound the crude product, which was purified by prep-HPLC (basic method, NH₄HCO₃) to give compound 64 (10.8 mg, 26%).

LC-MS(ESI): m/z=607.3[M+H]⁺.

### Synthetic Route of Compound 65

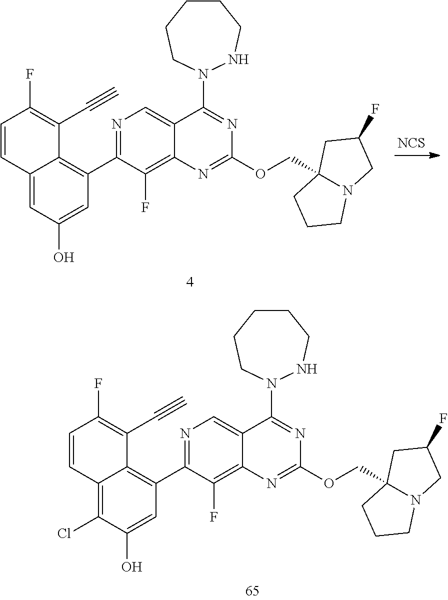

### Synthesis of Compound 65

Compound 4 (50 mg, 0.085 mmol) was dissolved in 5 mL of dichloromethane at room temperature, NCS (N-chlorosuccinimide) (17 mg, 0.13 mmol) was added and the reaction mixture was stirred at room temperature overnight. The reaction mixture was concentrated at reduced pressure and the residue was purified by flash column chromatography (DCM/MeOH=10:1) to obtain the crude product, which was further purified by prep-HPLC (basic method, NH₄HCO₃ system) to give compound 65 (3 mg, 5.7%). LC-MS (ESI): m/z=623.2 [M+H]⁺.

### Synthetic Route of Compound 66

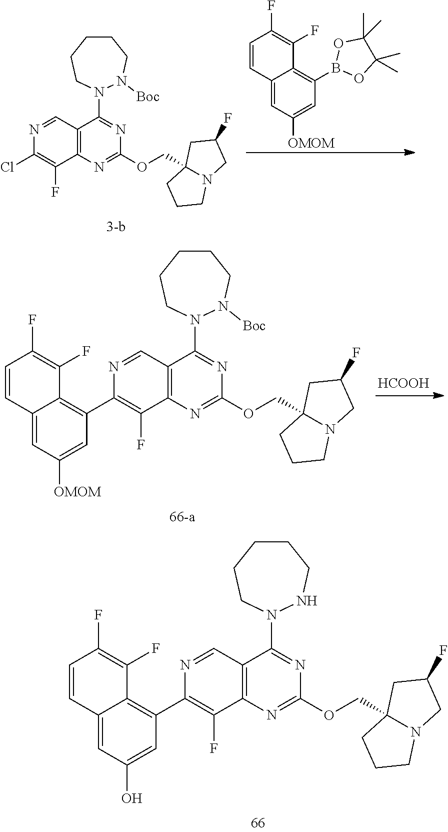

### Synthesis of Compound 66-a

To a microwave tube were added 3-b (100 mg, 0.19 mmol), 2-(7,8-difluoro-3-(methoxymethoxy)naphthalen-1-yl)-4,4,5,5-tetramethyl-1,3,2-dioxaborolane (97 mg, 0.28 mmol), potassium phosphate (118 mg, 0.56 mmol), cataCXium A Pd G3 (14 mg, 0.019 mmol), tetrahydrofuran (10 mL), water (1 mL). After degassed and purged with nitrogen for three times, the microwave tube was sealed and stirred at 65° C. overnight. The reaction mixture was concentrated to dryness and the residue was purified by column chromatography (mobile phase: methanol: dichloromethane=0-5%) to give compound 66-a (107 mg, 79%).

### Synthesis of Compound 66

Formic acid (4 mL) was combined with 66-a (30 mg, 0.041 mmol) at room temperature and the mixture was stirred at 35° C. overnight. The reaction mixture was concentrated to dryness, purified by prep-HPLC (ammonium bicarbonate) and lyophilized to give compound 66 (9 mg, 37%). LC-MS (ESI): m/z 583.3 (M+H)⁺.

### Synthetic Route of Compound 67

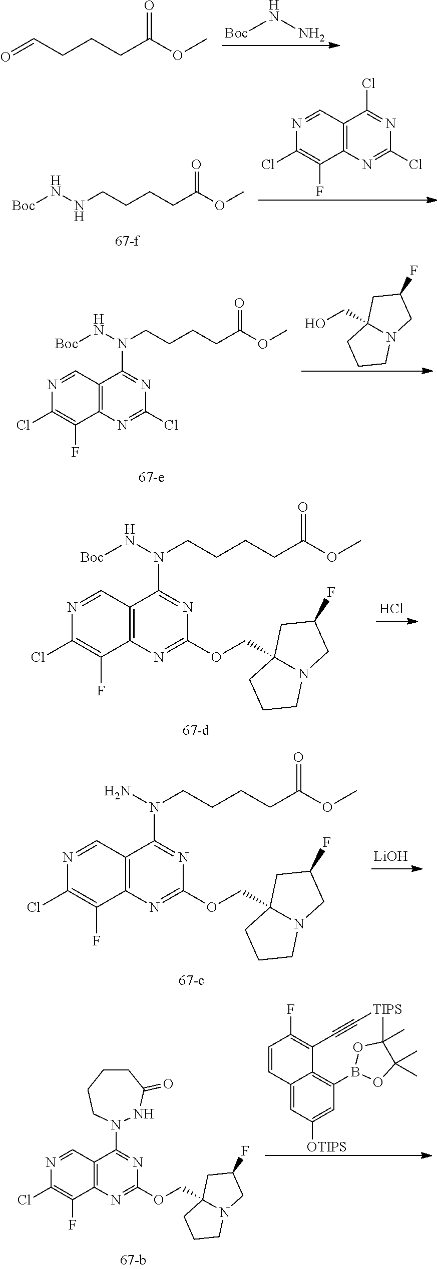

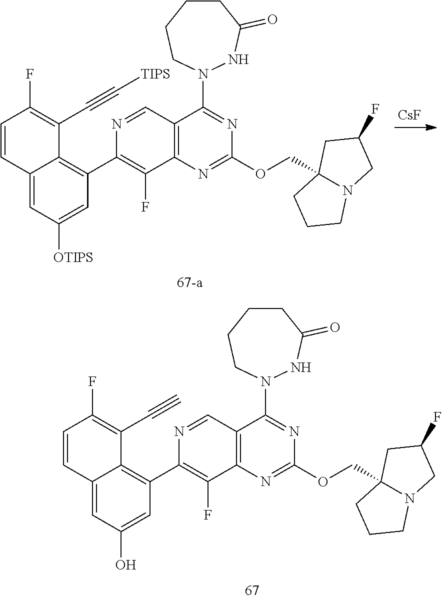

### Synthesis of Compound 67-f

A reaction vial charged with tert-butyl carbazate (2 g, 15.13 mmol), ethanol (80 mL) and methyl 5-oxovalerate (1.97 g, 15.14 mmol) was stirred at room temperature overnight under atmosphere of nitrogen. After addition of 10% Pd/C (1.6 g, 1.51 mmol), the mixture was degassed and purged with hydrogen for three times and stirred at room temperature overnight. The reaction mixture was filtered through celite, the filtrate was concentrated to dryness and the residue was purified by column chromatography (mobile phase ethyl acetate: petroleum ether=0-50%) to give compound 67-f (1.35 g, 36%). LC-MS (ESI): m/z 247.2 (M+H)⁺.

### Synthesis of Compound 67-e

A reaction vial charged with 67-f (300 mg, 1.22 mmol), dichloromethane (40 mL) was added 2,4,7-trichloro-8-fluoropyrido[4,3-d]pyrimidine (307 mg, 1.22 mmol) while cooled in a dry ice-acetone bath and stirred for 2 hr at room temperature under atmosphere of nitrogen. The reaction mixture was concentrated to dryness and the residue was purified by column chromatography (mobile phase: ethyl acetate: petroleum ether=0-30%) to give compound 67-e (293 mg, 52%). LC-MS (ESI): m/z 462.1 (M+H)⁺.

### Synthesis of Compound 67-d

A reaction vial charged with 67-e (293 mg, 0.63 mmol), ((2R,7aS)-2-fluorohexahydro-1H-pyrrolizin-7a-yl)methanol (303 mg, 1.90 mmol), 1,4-dioxane (20 mL) and DIPEA (410 mg, 3.17 mmol) was degassed and purged with nitrogen for three times, then stirred at 90° C. for three days. The reaction mixture was concentrated to dryness and the residue was purified by column chromatography (mobile phase: methanol: dichloromethane=0-5%) to give compound 67-d (348 mg, 94%). LC-MS (ESI): m/z 585.3 (M+H)⁺.

### Synthesis of Compound 67-c

A reaction vial charged with 67-d (348 mg, 0.595 mmol), acetonitrile (20 mL), hydrochloride/1,4-dioxane(4M, 2 mL, 8 mmol) was stirred at room temperature overnight. The reaction mixture was concentrated to dryness to give compound 67-c (crude), which was used directly in the next step without purification. LC-MS (ESI): m/z 485.3 (M+H)⁺.

### Synthesis of Compound 67-b

A reaction vial charged with 67-c (crude, 0.59 mmol), methanol (10 mL), tetrahydrofuran (32 mL), water (3 mL) and lithium hydroxide (249 mg, 5.94 mmol) was stirred at room temperature for 2 hours. The reaction mixture was adjusted to pH 4 with 1 M hydrochloric acid, removed the water by rotary evaporation, removed the water twice with ethanol and twice with acetonitrile. The residue was add acetonitrile (100 mL), HOBt (401 mg, 2.97 mmol), EDCI (683 mg, 3.56 mmol) and DIPEA (0.982 mL, 5.94 mmol), then stirred at room temperature overnight under atmosphere of nitrogen. Supplemented with HOBt (401 mg, 2.97 mmol), EDCI (683 mg, 3.56 mmol), DIPEA (0.982 mL, 5.94 mmol), stirred at room temperature overnight under atmosphere of nitrogen. Acetonitrile was removed by rotary evaporation and water was added, the aqueous phase was extracted with ethyl acetate (80 mL*2). The organic phase was washed with brine, dried over anhydrous sodium sulfate, concentrated to dryness, and the residue was purified by column chromatography (mobile phase: methanol: dichloromethane=0-10%) to give compound 67-b (40 mg, 15%). LC-MS (ESI): m/z 453.2(M+H)⁺.

### Synthesis of Compound 67-a

To a microwave tube were added 67-b (40 mg, 0.088 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (83 mg, 0.13 mmol), potassium phosphate (94 mg, 0.44 mmol), cataCXium A Pd G3 (6 mg, 0.009 mmol), tetrahydrofuran (10 mL) and water (1 mL). After sealed, degassed and purged with nitrogen for three times, the microwave tube was stirred at 65° C. overnight. The reaction mixture was concentrated to dryness and the residue was purified by column chromatography (mobile phase: methanol: dichloromethane=0-10%) to give compound 67-a (40 mg, 49%). LC-MS (ESI): m/z 915.4 (M+H)⁺.

### Synthesis of Compound 67

To 67-a (40 mg, 0.044 mmol) were added DMF (3 mL) and cesium fluoride (66 mg, 0.44 mmol) at room temperature and the mixture was stirred at room temperature overnight. The reaction mixture was filtered, and the filtrate was purified by prep-HPLC (ammonium bicarbonate) and immediately lyophilized to give compound 67 (6 mg, 23%). LC-MS (ESI): m/z 603.3 (M+H)⁺.

### Synthetic Route of Compound 68

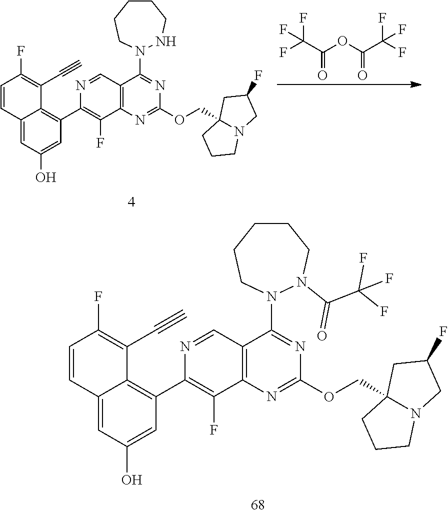 [SMILES: C#Cc1c(F)ccc2cc(O)cc(-c3ncc4c(N5CCCCCN5)nc(OC[C@@]56CCCN5C[C@H](F)C6)nc4c3F)c12 ; O=C(OC(=O)C(F)(F)F)C(F)(F)F ; C#Cc1c(F)ccc2cc(O)cc(-c3ncc4c(N5CCCCCN5C(=O)C(F)(F)F)nc(OC[C@@]56CCCN5C[C@H](F)C6)nc4c3F)c12]

### Synthesis of Compound 68

To a solution of compound 4 (125 mg, 0.21 mmol) in dichloromethane (10 mL) in an ice-water bath was added triethylamine (64 mg, 0.63 mmol), followed by slow addition of trifluoroacetic anhydride (66 mg, 0.32 mmol). The mixture was warmed to room temperature and stirred for 2 hours. Upon completion of the reaction, the organic solvent was removed by concentration under low temperature at reduced pressure. The crude was purified by flash column chromatography (mobile phase: methanol/dichloromethane, 0-10%) to give compound 68 (75 mg, 52%). LC-MS (ESI): m/z 685.7 [M+H]⁺; ¹H NMR (DMSO-d₆, 400 MHz): δ 10.19 (1H, d, J=7.2 Hz), 8.67 (1H, s), 7.95-8.04 (1H, m), 7.44-7.52 (1H, m), 7.41 (1H, s), 7.16-7.28 (1H, m), 5.30 (1H, d, J=54.4 Hz), 4.04-4.66 (4H, m), 3.51-3.98 (3H, m), 2.96-3.23 (3H, m), 2.77-2.93 (1H, m), 1.50-2.29 (12H, m).

### Synthetic Route of Compound 69

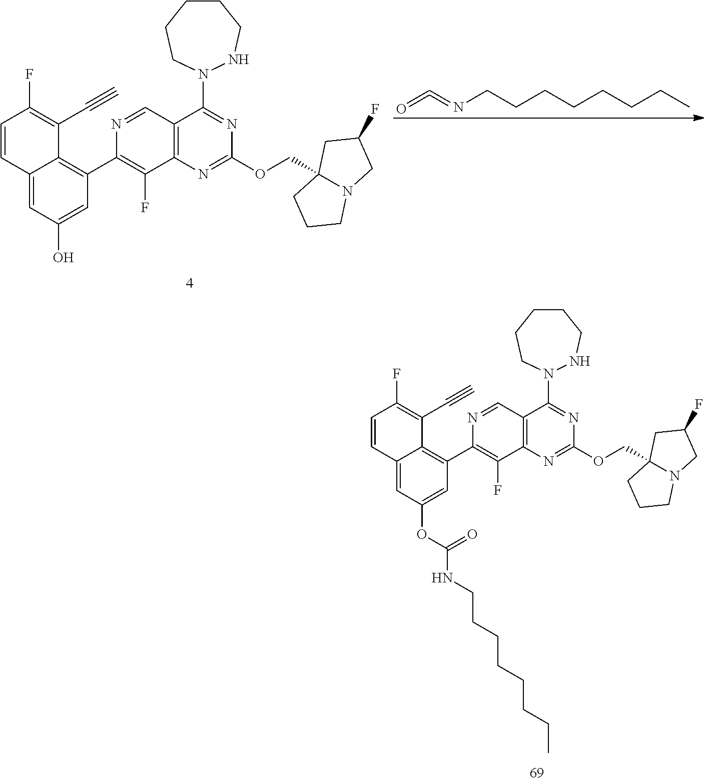

### Synthesis of Compound 69

To a solution of compound 4 (180 mg, 0.31 mmol) in dichloromethane (50 mL) in an ice-water bath was added pyridine (242 mg, 3.06 mmol), followed by slow addition of n-octyl isocyanate (66 mg, 0.32 mmol). The mixture was warmed to room temperature and stirred for 3 hours. Upon completion, the reaction was removed organic solvent by concentration under low temperature and reduced pressure. The mixture was first purified by flash column chromatography (mobile phase: methanol/dichloromethane, 0-10%) to obtain the crude product, and then purified by prep-HPLC to obtain product 69 (60 mg, 26%). LC-MS (ESI): m/z 744.4 [M+H]⁺; ¹H NMR (400 MHz, CDCl₃): δ 10.25 (1H, s), 7.60-7.90 (1H, m), 7.79 (1H, d, J=2.0 Hz), 7.40-7.50 (1H, m), 7.31 (1H, t, J=8.8 Hz), 5.30 (1H, d, J=53.6 Hz), 5.12 (1H, t, J=6.0 Hz), 4.01-4.39 (5H, m), 3.10-3.52 (7H, m), 2.95-3.05 (1H, m), 2.86 (1H, m), 2.13-2.41 (3H, m), 1.85-2.09 (5H, m), 1.68-1.85 (5H, m), 1.52-1.64 (3H, m), 1.18-1.43 (8H, m), 0.82-0.94 (3H, m).

### Synthetic Route of Compounds 70 and 71

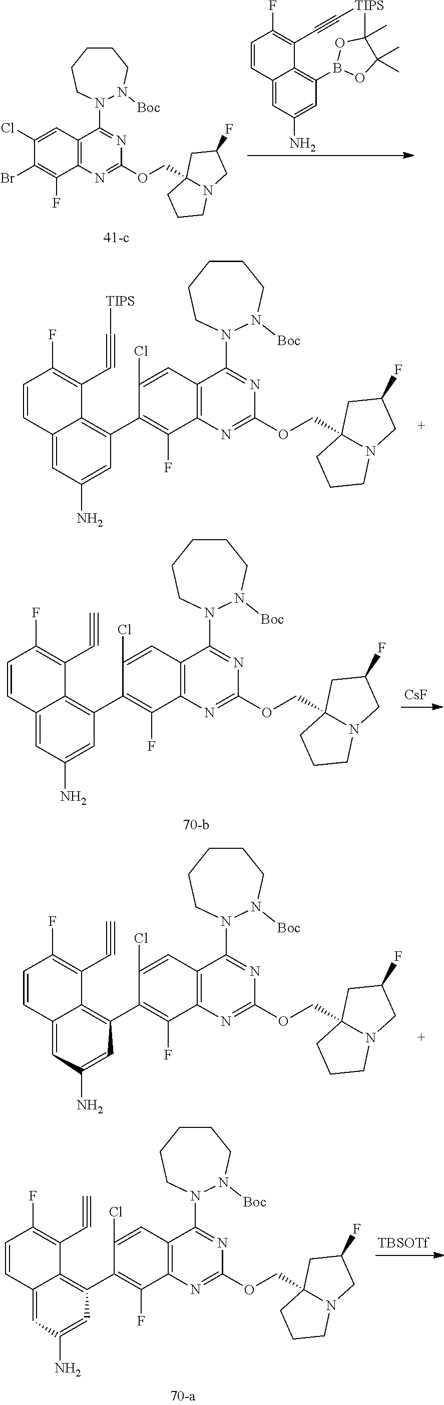

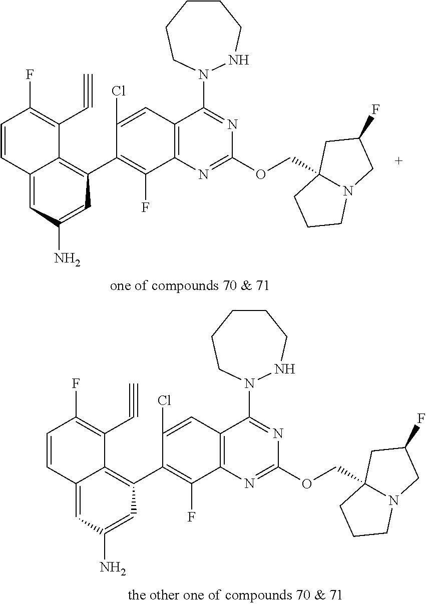

### Synthesis of Compound 70-b

To a microwave tube were added compound 41-c (200 mg, 0.32 mmol), 6-fluoro-4-(4,4,5,5-tetramethyl-1,3,2-dioxaborolan-2-yl)-5-((triisopropylsilyl)ethynyl)naphthalen-2-amine (227 mg, 0.49 mmol), palladium tetrakis(triphenylphosphine) (38 mg, 0.032 mmol), sodium carbonate (103 mg, 0.97 mmol), 1,4-dioxane (15 mL) and water (1.5 mL). After sealed, degassed and purged with nitrogen for three times, the microwave tube was stirred at 100° C. overnight. The reaction mixture was concentrated to dryness and the crude product was purified by column chromatography (mobile phase: methanol/dichloromethane, 0-10%) to give compound mixture 70-b (200 mg, 70%). LC-MS (ESI): m/z 877.1 (M+H)⁺; m/z 721.3 (M+H)⁺.

### Synthesis of Compound 70-a

To a solution of compound 70-b (200 mg, 0.23 mmol) in N,N-dimethylformamide (4 mL) was added cesium fluoride (346 mg, 2.28 mmol) at room temperature, and the mixture was stirred for 1 h at room temperature. A large amount of The reaction mixture was poured water and the aqueous phase was extracted with ethyl acetate (50 mL*2). The organic phase was washed with brine (30 mL*3), concentrated to dryness, and the residue was purified by column chromatography (mobile phase: methanol/dichloromethane, 0-10%) to give compound a mixture of the two isomers, 70-a (140 mg, 85%). LC-MS (ESI): m/z 721.6 (M+H)⁺.

### Synthesis of Compounds 70 and 71

To a solution of compound 70-a (140 mg, 0.19 mmol) in dichloromethane (4 mL) was added tert-butyldimethylsilyl trifluoromethanesulfonate (205 mg, 0.77 mmol) at room temperature, and the mixture was stirred for 1 h at room temperature. The reaction mixture was quenched by addition of saturated sodium bicarbonate solution (10 mL) and the mixture was stirred at room temperature overnight. Upon completion, the reaction mixture was extracted with dichloromethane (15 mL*2). The organic phase was washed with brine (10 mL*3), dried over anhydrous sodium sulfate, filtered, and the filtrate was concentrated to dryness. The crude was purified by prep-HPLC (basic method) and lyophilized to give compound two products. Compound 70 (30 mg, 25%), LC-MS (ESI): m/z 621.3 (M+H)⁺; Compound 71 (40 mg, 33%). LC-MS (ESI): m/z 621.3 (M+H)⁺.

### Synthetic Route of Compound 72

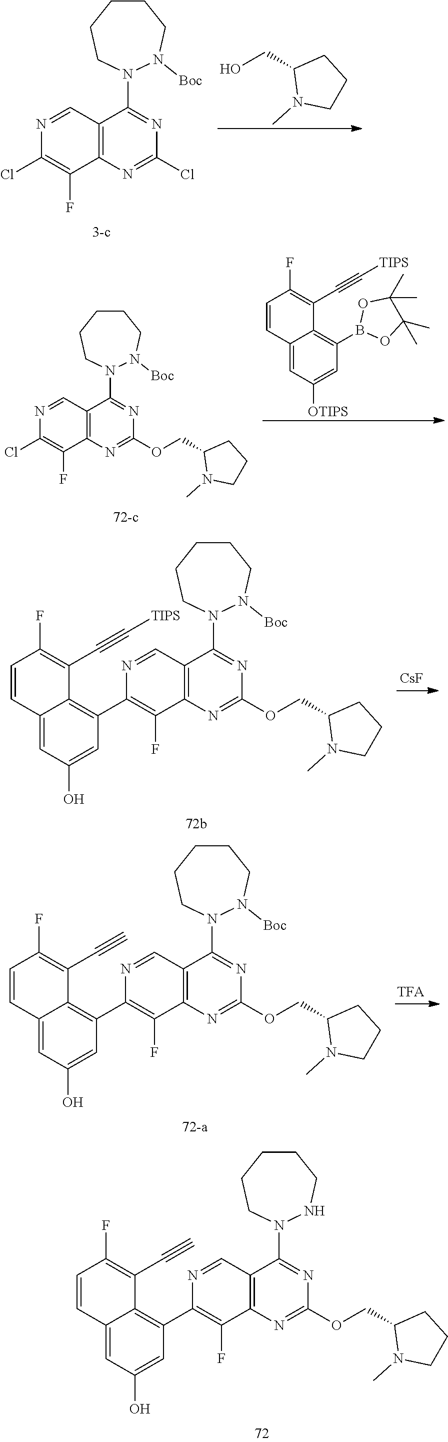

### Synthesis of Compound 72-c

To a solution of compound 3-c (100 mg, 0.24 mmol) in dichloromethane (15 mL) was added (S)-(-)-1-methyl-2-pyrrolidinomethanol (55 mg, 0.48 mmol) and then sodium tert-butoxide (115 mg, 1.20 mmol) in an ice-water bath. After addition, the reaction mixture was slowly warmed to room temperature and stirred at room temperature overnight. Water was added to the reaction mixture and the aqueous phase was extracted with dichloromethane (30 mL). The organic phase was dried over anhydrous sodium sulfate, filtered. The organic phase was concentrated to dryness, and the crude product was purified by flash column chromatography (mobile phase: dichloromethane:methanol=10:1/dichloromethane, 0-100%) to obtain 72-c (70 mg, 59%). LC-MS (ESI): m/z 495.4 (M+H)⁺.

### Synthesis of Compound 72-b

To a 10 mL microwave tube were added 72-c (70 mg, 0.14 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (106 mg, 0.17 mmol), methanesulfonic acid [n-butylbis(1-adamantyl)phosphine](2-amino-1,1′-biphenyl-2-yl)palladium(II) (10 mg, 0.014 mmol), potassium phosphate (90 mg, 0.42 mmol), tetrahydrofuran (2.5 mL) and water (0.5 mL). The microwave tube was sealed. After degassed and purged with nitrogen several times, the reaction mixture was stirred at 65° C. overnight. Water was added to the reaction mixture and the aqueous phase was extracted with ethyl acetate (20 mL). The organic phase was dried over anhydrous sodium sulfate, filtered, and the filtrate was concentrated to dryness and the crude was purified by flash column chromatography (mobile phase Dichloromethane: methanol=10:1/dichloromethane, 0-100%) to obtain 72-b (60 mg, 53%). LC-MS (ESI): m/z 801.7 (M+H)⁺.

### Synthesis of Compound 72-a

To a solution of 72-b (60 mg, 0.075 mmol) in N,N-dimethylformamide (8 mL) of was added cesium fluoride (95 mg, 0.63 mmol) at room temperature and the mixture was stirred at room temperature overnight. Water was added to the reaction mixture and the aqueous phase was extracted with ethyl acetate (30 mL). The organic phase was washed twice with brine, dried over anhydrous sodium sulfate, filtered, and the filtrate was concentrated to dryness to give compound 72-a (40 mg, 83%).

LC-MS (ESI): m/z 645.5 (M+H)⁺.

### Synthesis of Compound 72

Trifluoroacetic acid (2 mL) was added to a solution of 72-a (40 mg, 0.062 mmol) in dichloromethane (10 mL) at room temperature and the reaction mixture was stirred for 4 hours at this temperature. The reaction mixture was concentrated to dryness, neutralized by the addition of saturated aqueous sodium bicarbonate solution and the aqueous phase was extracted with dichloromethane (30 mL). The organic phase was dried over anhydrous sodium sulfate, filtered, and the filtrate was concentrated to dryness and the crude was purified by prep-HPLC to give compound 72 (3 mg, 9%).

LC-MS (ESI): m/z 545.3 (M+H)⁺.

### Synthetic Route of Compound 73

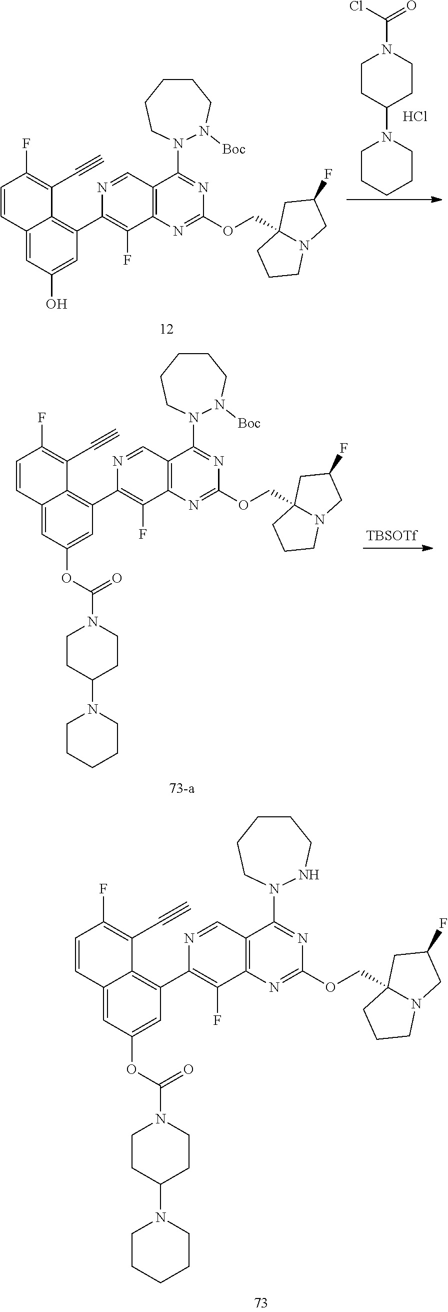

### Synthesis of Compound 73-a

To a solution of compound 12 (100 mg, 0.15 mmol) in N,N-dimethylformamide (6 mL) was added 1-chlorocarbonyl-4-piperidinylpiperidine hydrochloride (194 mg, 0.73 mmol) and potassium carbonate (201 mg, 1.45 mmol) at room temperature. The reaction mixture was stirred at 40° C. for 1 h. LCMS showed the reaction was complete, water was added to the reaction mixture and the aqueous phase was extracted with ethyl acetate (60 mL). The organic phase was washed twice with brine, dried over anhydrous sodium sulfate, filtered, and the filtrate was concentrated to dryness to give compound 73-a (119 mg, 93%). The crude product was used directly in the next step without purification. LC-MS (ESI): m/z 883.8 (M+H)⁺.

### Synthesis of Compound 73

To a solution of compound 73-a (119 mg, 0.13 mmol) in dichloromethane (10 mL) was added tert-butyldimethylsilyl trifluoromethanesulfonate (0.06 mL) in an ice-water bath, and the reaction mixture was stirred for 1 hr at this temperature, followed by the addition of saturated aqueous sodium bicarbonate and the reaction mixture was stirred at room temperature overnight. The reaction mixture was extracted with dichloromethane (30 mL). The organic phase was washed with brine, dried over anhydrous sodium sulfate, filtered, concentrated to dryness and the crude was purified by prep-HPLC to give compound 73 (23 mg, 22%). LC-MS (ESI): m/z 783.4(M+H)⁺.

### Synthetic Route of Compound 74

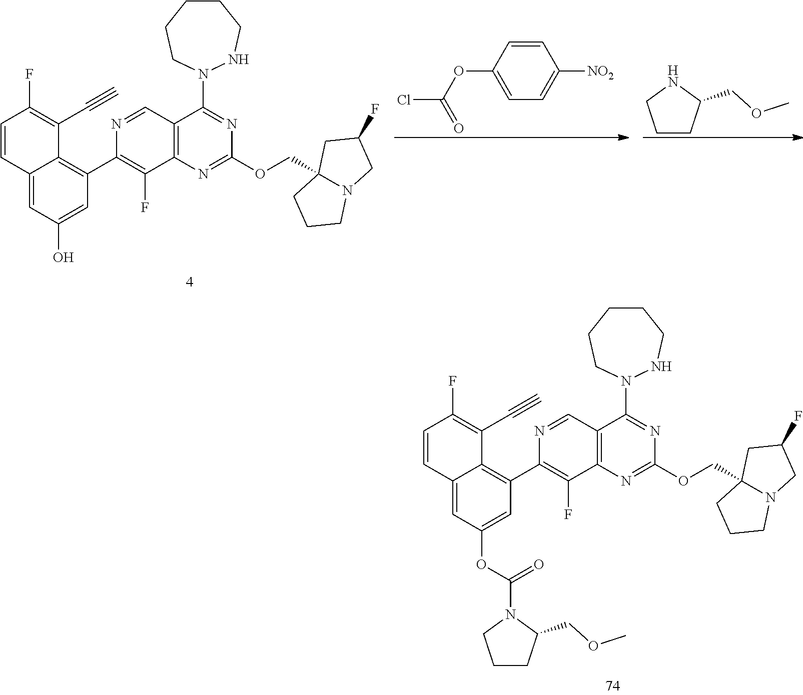

### Synthesis of Compound 74

To a solution of compound 4 (100 mg, 0.17 mmol) in DCM (2 mL) was added triethylamine (0.047 mL, 0.340 mmol) and p-nitrophenyl chloroformate (34 mg, 0.17 mmol) in an ice-salt bath and under protection of nitrogen. The reaction mixture was stirred at this temperature for 10 min, then was added (S)-2-(methoxymethyl)pyrazolidine (20 mg, 0.17 mmol) and slowly warmed to room temperature and the reaction mixture was stirred at room temperature overnight. Water was added to the reaction mixture and the aqueous phase was extracted with dichloromethane (30 ml). The organic phase was dried over anhydrous sodium sulfate, filtered, the filtrate was concentrated to dryness and the crude was purified by prep-HPLC to give compound 74 (81 mg, 65%). LC-MS (ESI): m/z 730.3 (M+H)⁺.

### Synthetic Route of Compound 75

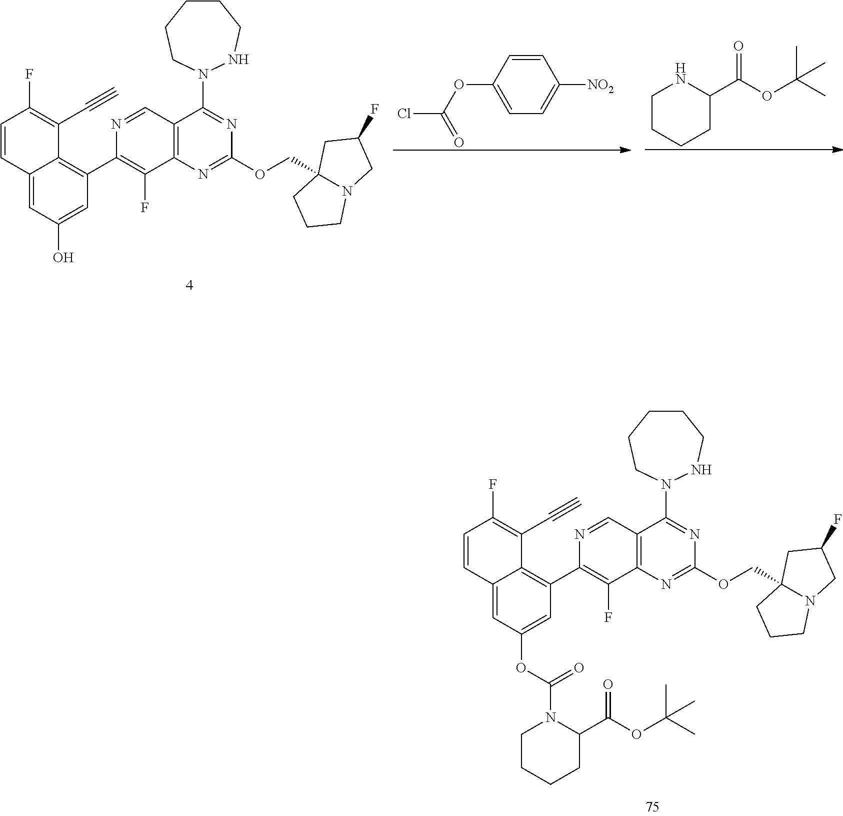

### Synthesis of Compound 75

Triethylamine (0.047 mL, 0.340 mmol) and p-nitrophenyl chloroformate (34 mg, 0.17 mmol) were added to a solution of compound 4 (100 mg, 0.17 mmol) in DCM (2 mL) under atmosphere of nitrogen in an ice-salt bath. The reaction mixture was stirred at this temperature for 10 min, then was added tert-Butyl 2-piperidinecarboxylate (31 mg, 0.17 mmol) and the reaction mixture was warmed to room temperature and stirred overnight. Water was added to the reaction mixture and the aqueous phase was extracted with dichloromethane (30 ml). The organic phase was dried over anhydrous sodium sulfate, filtered, the filtrate was concentrated to dryness and the crude was purified by prep-HPLC to give compound 75 (73 mg, 54%). LC-MS (ESI): m/z 800.3 (M+H)⁺.

### Synthetic Route of Compound 76

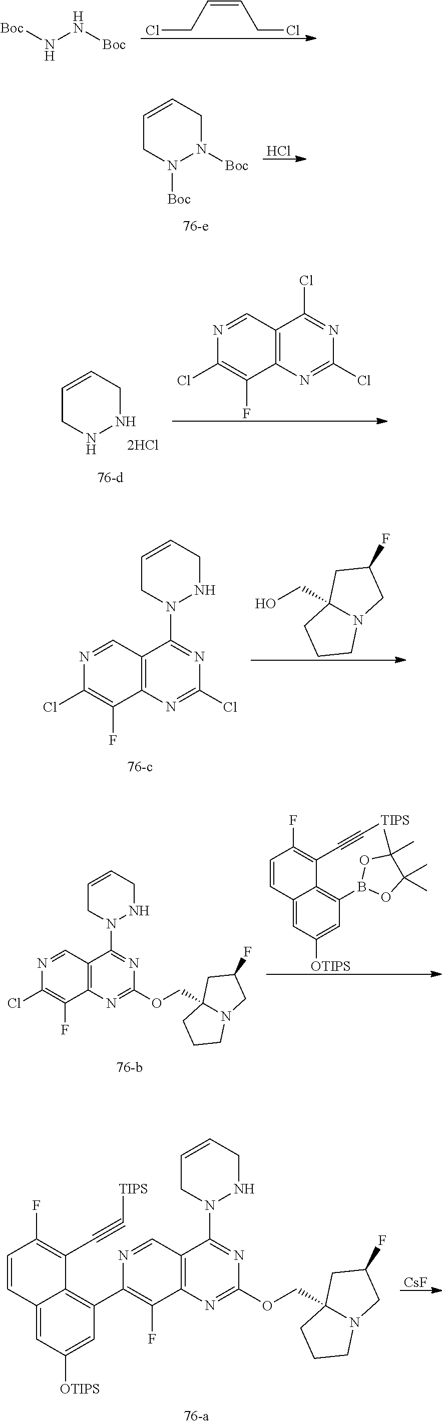

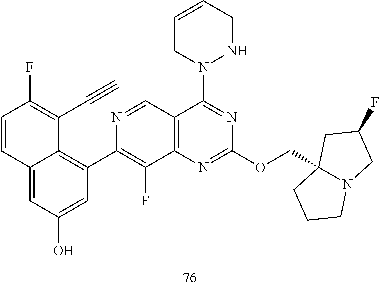 [SMILES: C#Cc1c(F)ccc2cc(O)cc(-c3ncc4c(N5CC=CCN5)nc(OC[C@@]56CCCN5C[C@H](F)C6)nc4c3F)c12]

### Synthesis of Compound 76-e

At room temperature, to a reaction vial were added di-tert-butyl hydrazodicarboxylate (2000 mg, 8.61 mmol), DMF (60 mL), cis-1,4-dichloro-2-butene (1076 mg, 8.61 mmol) and cesium carbonate (8416 mg, 25.83 mmol). After degassed and purged with nitrogen, the reaction mixture was stirred at room temperature overnight. The reaction mixture was filtered and the filtrate was added water and the aqueous phase was extracted with EA. The organic phases were combined, washed with water, brine, dried over anhydrous sodium sulfate, concentrated, and the residue was purified by column chromatography (mobile phase: PE/EA 10/0 to 8/2) to give compound 76-e (2000 mg, 82%).

LC-MS (ESI): m/z 302.2 (M+NH₄)⁺.

### Synthesis of Compound 76-d

76-e (500 mg, 1.76 mmol), acetonitrile (20 mL) and HCl/1,4-dioxane (1M, 5 mL) were added to a reaction vial at room temperature. The mixture was protected with N2 and stirred for 3 hours at room temperature. The reaction mixture was concentrated at room temperature to give the crude compound 76-d (430 mg).

### Synthesis of Compound 76-c

At room temperature, to a reaction vial were added 2,4,7-trichloro-8-fluoropyrido[4,3-d]pyrimidine (533 mg, 2.11 mmol), DCM (15 mL) and crude compound 76-d (430 mg, 1.76 mmol). After degassed and purged with nitrogen and cooled to 0° C., the mixture was was added DIPEA (1136 mg, 8.79 mmol). The reaction mixture was naturally warmed to room temperature and stirred overnight. Water was added to the reaction mixture and the aqueous phase was extracted with DCM. The organic phases were combined, dried over anhydrous sodium sulfate, filtered, concentrated and the residue was purified by column chromatography (mobile phase: PE/EA 10/0 to 8/2) to give compound 76-c (430 mg, 81%).

### Synthesis of Compound 76-b

At room temperature, to a reaction vial were added 76-c (430 mg, 1.43 mmol), ((2R,7aS)-2-fluorohexahydro-1H-pyrrolizin-7a-yl)methanol (342 mg, 2.15 mmol), 1,4-dioxane (5 mL) and DIPEA (741 mg, 5.73 mmol). After degassed and purged with nitrogen, the reaction mixture was stirred at 90° C. overnight. The reaction mixture was concentrated and the residue was purified by column chromatography (mobile phase: DCM/MeOH 10/0 to 10/1) to give compound 76-b (600 mg, 99%). LC-MS (ESI): m/z 423.3 (M+H)⁺.

### Synthesis of Compound 76-a

76-b (150 mg, 0.35 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (266 mg, 0.43 mmol), cataCXium A Pd G3 (52 mg, 0.07 mmol), potassium phosphate (226 mg, 1.07 mmol), THF (2 mL), and water (0.5 mL) were combined in a sealed tube. After degassed and purged with nitrogen, the reaction mixture was stirred at 65° C. overnight. The reaction mixture was concentrated and the residue was purified by column chromatography (mobile phase: DCM/MeOH 10/0 to 10/1) to give compound 76-a (220 mg, 70%).

### Synthesis of Compound 76

76-a (220 mg, 0.25 mmol), DMF (3 mL) and cesium fluoride (377 mg, 2.48 mmol) were added to a reaction vial at room temperature. The mixture was stirred at room temperature for 3 hours.

Filtered, and the filtrate was purified by reversed-phase column (mobile phase: water (0.01 M NH₄HCO₃)/acetonitrile 10/0 to 1/9) to give compound 76 (50 mg, 35%). LC-MS (ESI): m/z 573.4 (M+H)⁺; ¹H NMR (400 MHz, CD₃OD): δ 10.46 (1H, s), 10.04 (1H, s), 7.91-7.80 (2H, m), 7.43-7.18 (4H, m), 5.60-5.54 (1H, m), 5.30 (1H, d, J=53.2 Hz), 4.39-4.19 (3H, m), 3.40 (2H, t, J=6.0 Hz), 3.33 (1H, d, J=3.2 Hz), 3.27-3.13 (3H, m), 3.09-2.96 (1H, m), 2.50-1.80 (7H, m).

### Synthetic Route of Compound 77

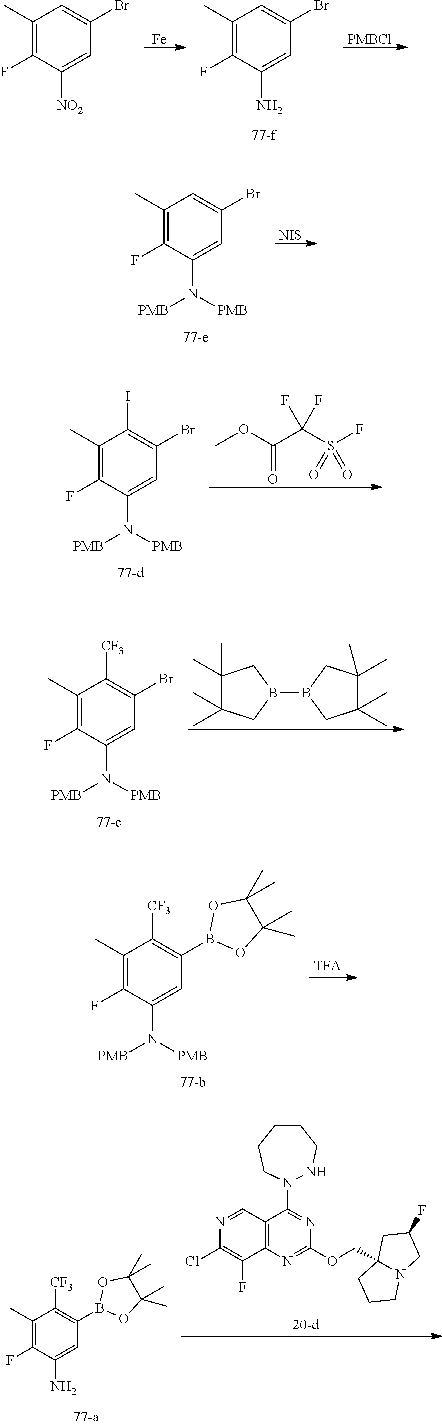

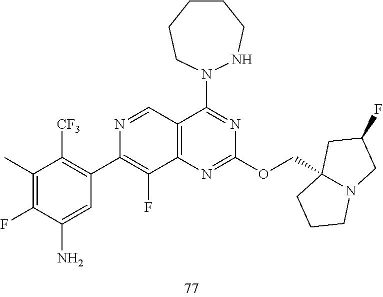 [SMILES: Cc1c(F)c(N)cc(-c2ncc3c(N4CCCCCN4)nc(OC[C@@]45CCCN4C[C@H](F)C5)nc3c2F)c1C(F)(F)F]

### Synthesis of Compound 77-f

At room temperature, to a reaction flask were added 5-bromo-2-fluoro-1-methyl-3-nitrobenzene (2500 mg, 10.68 mmol), ethanol (80 mL), water (40 mL), iron powder (5965 mg, 106.83 mmol) and ammonium chloride (5714 mg, 106.83 mmol). After degassed and purged with nitrogen, the reaction mixture was stirred at 100° C. for 2 hours. The reaction mixture was concentrated to remove ethanol, water and EA were added. Filtered and the filtrate was extracted with EA. The organic phases were combined, washed with water, brine, dried over anhydrous sodium sulfate, concentrated and the residue was purified by column chromatography (mobile phase: PE/EA 10/0 to 8/2) to give compound 77-f (1900 mg, 87%).

### Synthesis of Compound 77-e

At room temperature, to a reaction vial were added 77-f (1900 mg, 9.31 mmol) and DMF (15 mL). After degassed and purged with nitrogen and cooled down to 0° C., the mixture was added NaH (60%, 1490 mg, 37.25 mmol), stirred for half an hour, was added 4-methoxybenzyl chloride (PMBCl) (4375 mg, 27.94 mmol). The reaction mixture was naturally warmed to room temperature for 3 hours. Water was added to the reaction mixture and the aqueous phase was extracted with EA. The organic phases were combined, dried over anhydrous sodium sulfate, concentrated, and the residue was purified by column chromatography (mobile phase: PE/EA 10/0 to 8/2) to give compound 77-e (3600 mg, 87%).

### Synthesis of Compound 77-d

At room temperature, to a reaction vial were added 77-e (3600 mg, 8.10 mmol), acetic acid (25 mL), and NIS (N-iodosuccinimide) (1823 mg, 8.10 mmol). After degassed and purged with nitrogen, the reaction mixture was stirred for 1 hour at room temperature. The reaction mixture was concentrated, neutralized with aqueous sodium bicarbonate and the aqueous phase was extracted with EA. The organic phases were combined, washed with water, dried over anhydrous sodium sulfate, concentrated and the residue was purified by column chromatography (mobile phase: PE/EA 10/0 to 9/1) to give compound 77-d (1900 mg, 41%).

### Synthesis of Compound 77-c

At room temperature, to a reaction vial were added 77-d (1900 mg, 3.33 mmol), methyl fluorosulfonyl difluoroacetate (3200 mg, 16.66 mmol), DMAc (20 mL) and CuI (3173 mg, 16.66 mmol). After degassed and purged with nitrogen, the reaction mixture was stirred at 90° C. for 2 h. The reaction mixture was cooled to room temperature, water was added, the aqueous phase was extracted with EA. The organic phases were combined, dried, concentrated and the residue was purified by column chromatography (mobile phase: PE/EA 10/0 to 10/1) to give compound 77-c (1300 mg, 76%). LC-MS (ESI): m/z 512.1 (M+H)⁺.

### Synthesis of Compound 77-b

At room temperature, to a reaction vial were added 77-c (1300 mg, 2.54 mmol), pinacol bis(boronic acid) ester (1933 mg, 7.61 mmol), Pd(dppf)Cl₂ (371 mg, 0.51 mmol), potassium acetate (996 mg, 10.15 mmol), 1,4-dioxane (10 mL). After degassed and purged with nitrogen, the reaction mixture was stirred at 100° C. for 3 hours. The reaction mixture was concentrated and the residue was purified by column chromatography (mobile phase: PE/EA 10/0 to 10/1) to give compound 77-b (1300 mg, 92%). LC-MS (ESI): m/z 560.3 (M+H)⁺.

### Synthesis of Compound 77-a

77-b (1300 mg, 2.32 mmol), TFA (10 mL) were added to a reaction vial at room temperature. After degassed and purged with nitrogen, the reaction mixture was stirred for 3 h at room temperature. The reaction mixture was concentrated at room temperature, alkalized by addition of methanol solution of ammonia, concentrated, and the residue was purified by column chromatography (mobile phase: DCM/MeOH 10/0 to 10/1) to give compound 77-a (450 mg, 61%).

### Synthesis of Compound 77

To a sealed tube at room temperature was added 77-a (200 mg, 0.63 mmol), 20-d (330 mg, 0.75 mmol), cataCXium A Pd G3 (46 mg, 0.06 mmol), potassium phosphate (399 mg, 1.88 mmol), THF (4 mL) and water (1 mL). After degassed and purged with nitrogen, the reaction mixture was stirred at 65° C. for 3 hours. The reaction mixture was concentrated, dissolved by adding methanol, filtered and the filtrate was purified by prep-HPLC to give compound 77 (56 mg, 15%). LC-MS (ESI): m/z 596.6 (M+H)⁺.

### Synthetic Route of Compound 78

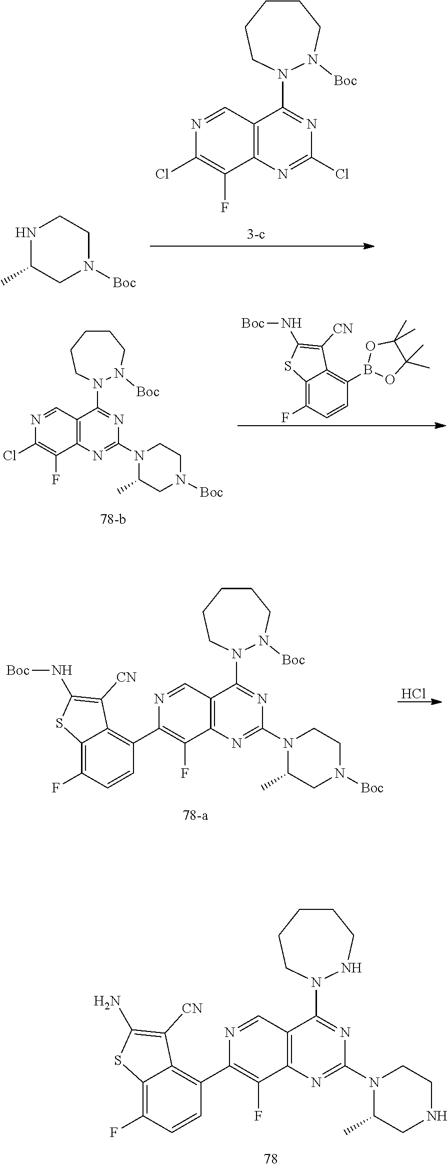

### Synthesis of Compound 78-b

At room temperature, to a reaction vial were added 3-c (200 mg, 0.48 mmol), (S)-4-N-Boc-2-methylpiperazine (192 mg, 0.96 mmol), 1,4-dioxane (10 mL), and DIPEA (248 mg, 1.92 mmol). After degassed and purged with nitrogen, the reaction mixture was stirred at 90° C. overnight. The reaction mixture was concentrated and the residue was purified by column chromatography (mobile phase: PE/EA 10/0 to 1/1) to give compound 78-b (230 mg, 83%). LC-MS (ESI): m/z 580.4 (M+H)⁺.

### Synthesis of Compound 78-a

At room temperature, to a microwave tube were added 78-b (100 mg, 0.17 mmol), tert-butyl 3-cyano-7-fluoro-4-(4,4,5,5-tetramethyl-1,3,2-dioxaborolan-2-yl)benzo[b]thiophen-2-yl)carbamate 108 mg, 0.26 mmol), bis(diphenylphosphine phenyl ether)palladium(II) dichloride (25 mg, 0.03 mmol), cesium carbonate (112 mg, 0.34 mmol) and toluene (2 mL). The mixture was degassed and purged with nitrogen and microwave stirred at 100° C. overnight. The reaction mixture was concentrated and the residue was purified by column chromatography (mobile phase: PE/EA 10/0 to 0/10) to give compound 78-a (70 mg, 49%). LC-MS (ESI): m/z 836.5 (M+H)⁺.

### Synthesis of Compound 78

At room temperature, to a reaction vial were added 78-a (70 mg, 0.08 mmol), acetonitrile (15 mL), and HCl/1,4-dioxane (1M, 4 mL). The resulting mixture was stirred at room temperature for 3 hours.

Concentrate at room temperature, and the residue was adjusted to basic with a methanolic solution of ammonia and concentrated, then dissolved with methanol, and the residue was purified by prep-HPLC to give compound 78 (11 mg, 25%). LC-MS (ESI): m/z 536.4 (M+H)⁺.

### Synthetic Route of Compound 79

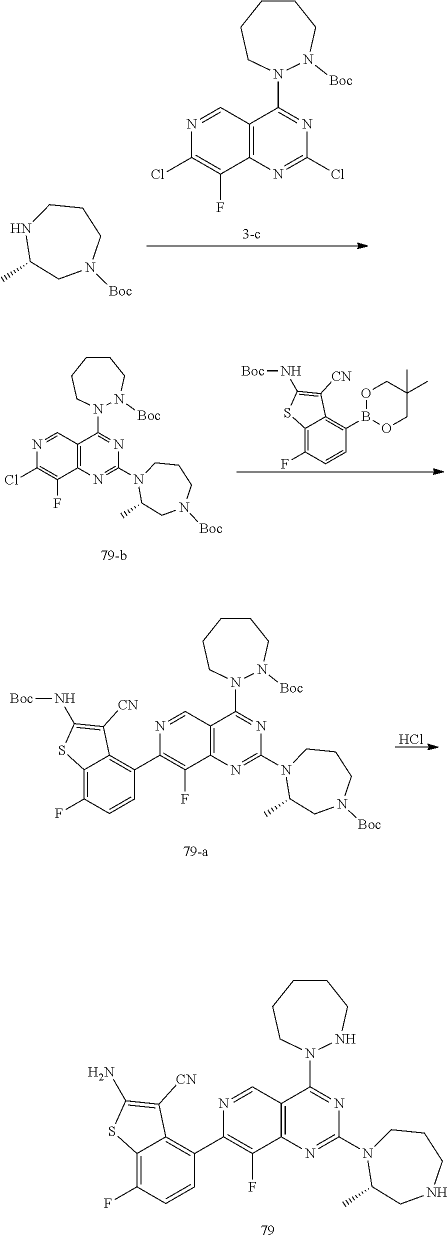

### Synthesis of Compound 79-b

At room temperature, to a reaction vial were added 3-c (200 mg, 0.48 mmol), tert-butyl (S)-3-methyl-1,4-diazepane-1-carboxylate (206 mg, 0.96 mmol), 1,4-dioxane (10 mL) and DIPEA (248 mg, 1.92 mmol). After degassed and purged with nitrogen, the reaction mixture was stirred at 90° C. overnight. The reaction mixture was concentrated and the residue was purified by column chromatography (mobile phase: PE/EA 10/0 to 1/1) to give compound 79-b (240 mg, 84%). LC-MS (ESI): m/z 594.3 (M+H)*.

### Synthesis of Compound 79-a

At room temperature, to a microwave tube were added 79-b (100 mg, 0.17 mmol), tert-butyl (3-cyano-4-(5,5-dimethyl-1,3,2-dioxaborolan-2-yl)-7-fluorobenzo[b]thiophen-2-yl)carbamate(204 mg, 0.50 mmol), bis(diphenylphosphine phenyl ether) palladium(II) dichloride (24 mg. 0.03 mmol), cesium carbonate (165 mg, 0.51 mmol) and toluene (2 mL). After degassed and purged with nitrogen, the mixture was microwaved at 120° C. for 48 hours. The reaction mixture was concentrated and the residue was purified by column chromatography (mobile phase: PE/EA 10/0 to 0/10) to give compound 79-a (80 mg, 56%).

### Synthesis of Compound 79

At room temperature, to a reaction vial were added 79-a (80 mg, 0.09 mmol), acetonitrile (15 mL) and HCl/1,4-dioxane (1M, 4 mL). The reaction mixture was stirred at room temperature for 3 hours. Concentrate at room temperature, and the residue was adjusted to basic with a methanol solution of ammonia and concentrate, then dissolved with methanol, and the residue was purified by prep-HPLC to give compound 79 (8 mg, 15%). LC-MS (ESI): m/z 550.3 (M+H)⁺.

### Synthetic Route of Compound 80

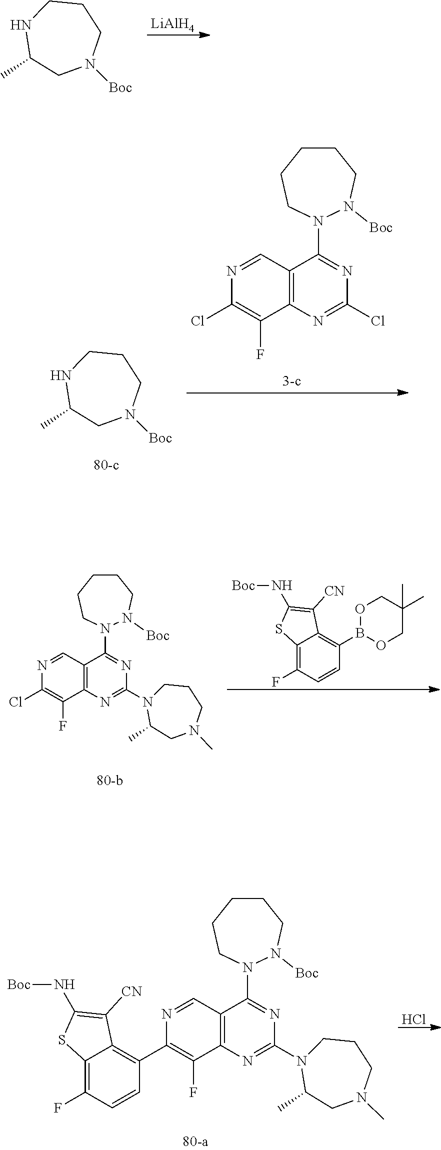

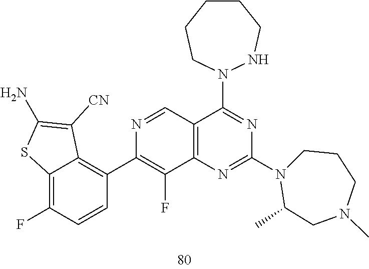 [SMILES: C[C@H]1CN(C)CCCN1c1nc(N2CCCCCN2)c2cnc(-c3ccc(F)c4sc(N)c(C#N)c34)c(F)c2n1]

### Synthesis of Compound 80-c

At room temperature, to a reaction vial were added tert-butyl (S)-3-methyl-1,4-diazepane-1-carboxylate (400 mg, 1.87 mmol) and THF (10 mL). After degassed and purged with nitrogen, the above mixture was cooled down to 0° C., was added a solution of lithium aluminum hydride in tetrahydrofuran (1 M, 5.6 mL) dropwise. The reaction mixture was stirred at room temperature for 48 hours, then was added sodium sulfate decahydrate and stirred for 1 h. The filtrate was filtered and the filter cake was washed with THF. The filtrate was concentrated to give the crude compound 80-c (300 mg). LC-MS (ESI): m/z 128.8 (M+H)⁺.

### Synthesis of Compound 80-b

At room temperature, to a reaction vial were added 3-c (388 mg, 0.93 mmol), 80-c (150 mg, 0.93 mmol), 1,4-dioxane (10 mL) and DIPEA (482 mg, 3.73 mmol). After degassed and purged with nitrogen, the reaction mixture was stirred at 90° C. overnight. The reaction mixture was concentrated and the residue was purified by column chromatography (mobile phase: DCM/MeOH 10/0 to 10/1) to give compound 80-b (150 mg, 32%). LC-MS (ESI): m/z 508.4 (M+H)⁺.

### Synthesis of Compound 80-a

At room temperature, to a microwave tube were added 80-b (150 mg, 0.30 mmol), tert-butyl (3-cyano-4-(5,5-dimethyl-1,3,2-dioxaborolan-2-yl)-7-fluorobenzo[b]thiophen-2-yl)carbamate (358 mg, 0.89 mmol), bis(diphenylphosphine phenyl ether) palladium(II) dichloride (42 mg. 0.06 mmol), cesium carbonate (289 mg, 0.89 mmol) and toluene (3 mL). The mixture was degassed and purged with nitrogen, then microwaved at 120° C. for 48 hours. The reaction mixture was concentrated and the residue was purified by column chromatography (mobile phase: DCM/MeOH 10/0 to 10/1) to give compound 80-a (70 mg, 31%). LC-MS (ESI): m/z 764.9 (M+H)⁺.

### Synthesis of Compound 80

At room temperature, to a reaction vial were added 80-a (70 mg, 0.09 mmol), acetonitrile (15 mL) and HCl/1,4-dioxane (1M, 4 mL). The reaction mixture was stirred at room temperature for 3 hours. Concentrate at room temperature, and the residue was adjusted to basic with a methanolic solution of ammonia, and concentrate, then dissolved with methanol, and the residue was purified by prep-HPLC to give compound 80 (2.7 mg, 5%). LC-MS (ESI): m/z 564.4 (M+H)⁺.

### Synthetic Route of Compound 81

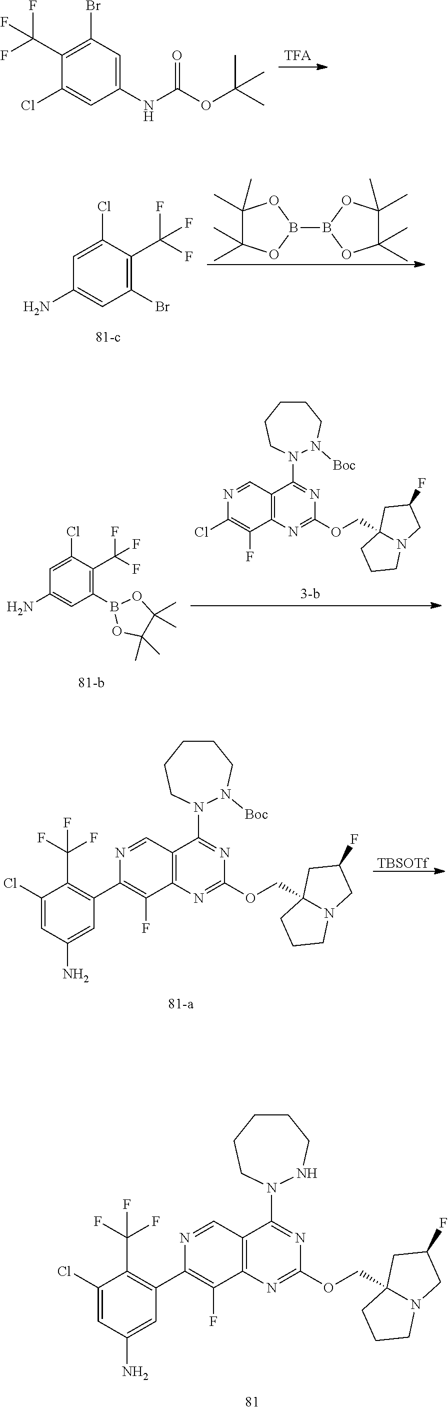

### Synthesis of Compound 81-c

To a reaction flask were added tert-butyl 3-bromo-5-chloro-4-trifluoromethylaniline formate (1 g, 2.67 mmol), dichloromethane (9 mL) and trifluoroacetic acid (3 mL, 40.04 mmol). The reaction mixture was stirred at room temperature for 1 hour, concentrated to dryness, diluted by adding dichloromethane/methanol (10:1, 10 mL), the pH was neutralized to 8-9 by adding saturated aqueous ammonium bicarbonate solution, partitioned, and the aqueous phase was extracted with dichloromethane/methanol (10:1, 10 mL). The organic phases were combined, dried over anhydrous sodium sulfate, and the residue was purified by column chromatography (mobile phase: ethyl acetate/petroleum ether=0-20%) to give compound 81-c (397 mg, 54%). LC-MS (ESI): m/z 273.9 (M+H)⁺.

### Synthesis of Compound 81-b

To a reaction flask in an ice-water bath was added 81-c (394 mg, 1.44 mmol), pinacol ester of bisboronic acid (1.094 g, 4.31 mmol), potassium acetate (423 mg, 4.31 mmol), Pd(dppf)Cl₂—CH₂Cl₂ (59 mg, 0.072 mmol) and 1,4- dioxane (6 mL). The mixture was degassed and purged with nitrogen for three times, then stirred at 90° C. for 2 hours. The reaction mixture was supplemented with Pd(dppf)Cl₂—CH₂Cl₂ (59 mg, 0.072 mmol), degassed and purged with nitrogen for three times, stirred at 90° C. for 1.5 hours. The reaction mixture was concentrated to dryness and the residue was purified by column chromatography (mobile phase: ethyl acetate: petroleum ether=0-20%) to give compound 81-b (275 mg, 60%). LC-MS (ESI): m/z 321.9 (M+H)⁺.

### Synthesis of Compound 81-a

To a microwave tube were added 3-b (100 mg, 0.19 mmol), 81-b (78 mg, 0.24 mmol), [n-butylbis(1-adamantyl)phosphine](2-amino-1,1′-biphenyl-2-yl)palladium(II) mesylate (27 mg, 0.04 mmol), potassium phosphate (118 mg, 0.56 mmol), tetrahydrofuran (1.0 mL) and water (0.2 mL). The reaction mixture was sealed, degassed and purged with nitrogen for three times, stirred at 65° C. for 1 hour. Dichloromethane (2 mL) and water (1 mL) were added to the reaction mixture, partitioned and the aqueous phase was extracted with dichloromethane (2 mL). The organic phases were combined, dried over anhydrous sodium sulfate, filtered, and the filtrate was concentrated to dryness and the residue was purified by column chromatography (mobile phase: ethyl acetate: petroleum ether=0-20%) to give compound 81-a (62 mg, 48%). LC-MS (ESI): m/z 698.2 (M+H)⁺.

### Synthesis of Compound 81

To a solution of 81-a (70 mg, 0.10 mmol) in dichloromethane (2.4 mL) was added tert-butyldimethylsilyl trifluoromethanesulfonate (398 mg, 1.50 mmol) in an ice-water bath, and the mixture was stirred for 0.5 hr at 0-5° C. The reaction mixture was added saturated aqueous sodium bicarbonate solution (4 mL), stirred at room temperature for 20 min, then was extracted with dichloromethane/methanol (10:1) (5 mL*2). The organic phase was dried over anhydrous sodium sulfate, filtered, concentrated to dryness, purified by column chromatography (mobile phase: methanol: dichloromethane=0-12%), concentrated, lyophilized to give compound 81 (38 mg, 63%).

LC-MS (ESI): m/z 598.3 (M+H)⁺; ¹H NMR (400 MHz, d₆-DMSO) δ 10.22 (s, 1H), 6.88 (d, J=2.3 Hz, 1H), 6.48 (d, J=2.3 Hz, 1H), 6.32 (s, 2H), 5.89 (t, J=6.1 Hz, 1H), 5.32 (d, J=54.3 Hz, 1H), 4.33-3.83 (m, 4H), 3.04 (t, J=59.2 Hz, 6H), 2.22-1.99 (m, 3H), 1.97-1.76 (m, 5H), 1.75-1.59 (m, 4H).

### Synthetic Route of Compound 82

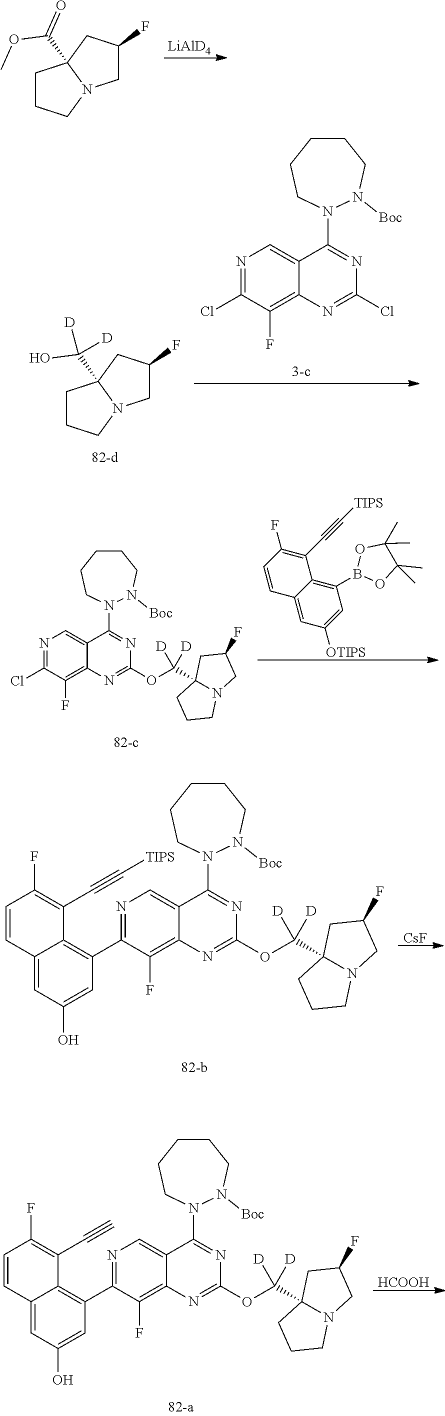

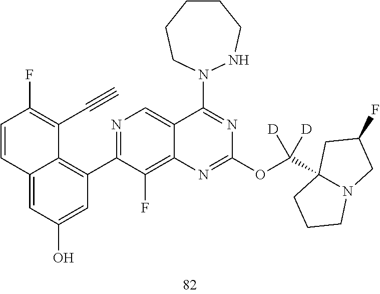 [SMILES: [2H]C([2H])(Oc1nc(N2CCCCCN2)c2cnc(-c3cc(O)cc4ccc(F)c(C#C)c34)c(F)c2n1)[C@@]12CCCN1C[C@H](F)C2]

### Synthesis of Compound 82-d

To a reaction flask were added methyl (2R,7aS)-2-fluorotetrahydro-1H-pyrrolizine-7a(5H)-carboxylate (400 mg, 2.14 mmol) and tetrahydrofuran (10 mL). After stirred in an ice-water bath for 10 min, the reaction mixture was added lithium aluminum deuterium (126 mg, 2.99 mmol), and stirred for 12 h at room temperature. The reaction mixture was quenched by the addition of sodium sulfate decahydrate until no bubbles were emitted, filtered, and the filtrate was concentrated and the residue was purified by column chromatography (MeOH/DCM=0/100,1/10) to give compound 82-d (260 mg, 75%). LC-MS (ESI): m/z=162.2 (M+H)⁺.

### Synthesis of Compound 82-c

To a reaction flask were added 82-d (260 mg, 1.61 mmol), dichloromethane (20 mL), 3-c (700 mg, 1.68 mmol), sodium tert-butoxide (162 mg, 1.68 mmol), respectively, and the mixture was stirred at 0° C. for 3 hr under protection of nitrogen. Water (20 mL) was added to the reaction mixture and the aqueous phase was extracted with dichloromethane (30 mL*3). The organic phase was concentrated and the residue was purified by column chromatography (MeOH/DCM=1/10) to afford compound 82-c (560 mg, 64%). LC-MS (ESI): m/z=541.1(M+H)⁺.

### Synthesis of Compound 82-b

A reaction vial charged with 82-c (320 mg, 0.59 mmol), tetrahydrofuran (18 mL), water (2 mL), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (554 mg, 0.89 mmol), potassium phosphate (439 mg, 2.07 mmol) and cataCXium A Pd G3 (2.48 mg, 0.003 mmol). After degassed and purged with nitrogen for three times, the reaction vial was placed in an oil bath prestirred at 60° C. and stirred for 12 hours. The reaction mixture was concentrated to dryness and the residue was purified by column chromatography (mobile phase: methanol/dichloromethane=0/100; 1/10) to give compound 82-b (270 mg, 54%).

### Synthesis of Compound 82-a

To a reaction flask, 82-b (270 mg, 0.32 mmol), DMF (20 mL) and cesium fluoride (800 mg, 5.27 mmol) were added. The reaction mixture was stirred at room temperature for 6 hours, then was added water (100 mL) and the aqueous phase was extracted with ethyl acetate (100 mL*3). The organic phase was concentrated and passed through the column layer (MeOH/DCM=0/100, 1/10) to give compound 82-a (170 mg, 77%).

### Synthesis of Compound 82

A reaction vial charged with 82-a (170 mg, 0.25 mmol) and formic acid (20 mL) was stirred at room temperature for 6 hours. The reaction mixture was concentrated at room temperature and the residue was purified by prep-HPLC to afford compound 82 (32 mg, 22%), LC-MS (ESI): m/z 591.3(M+H)⁺.

### Synthetic Route of Compound 83

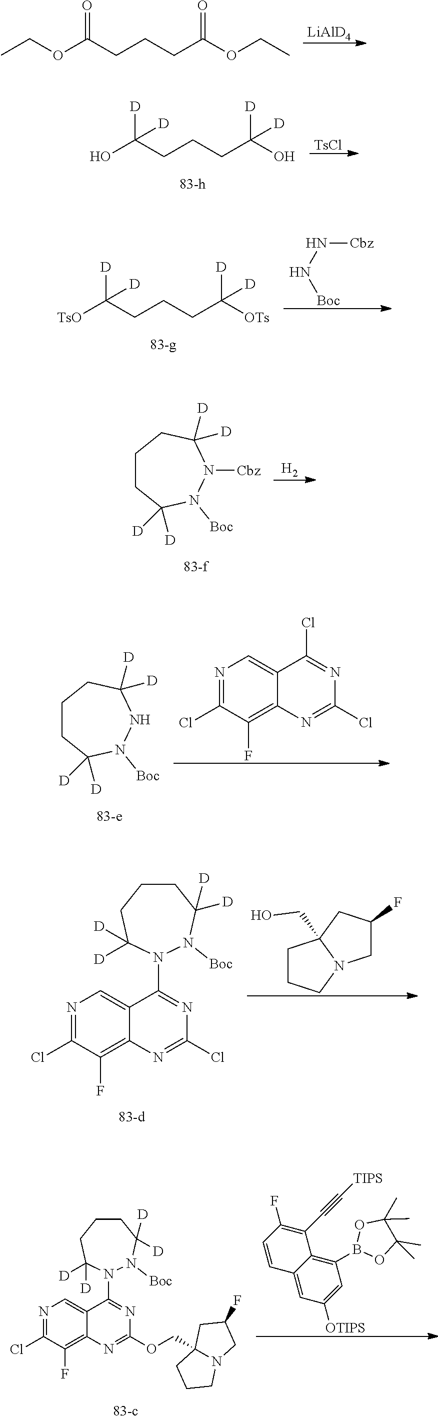

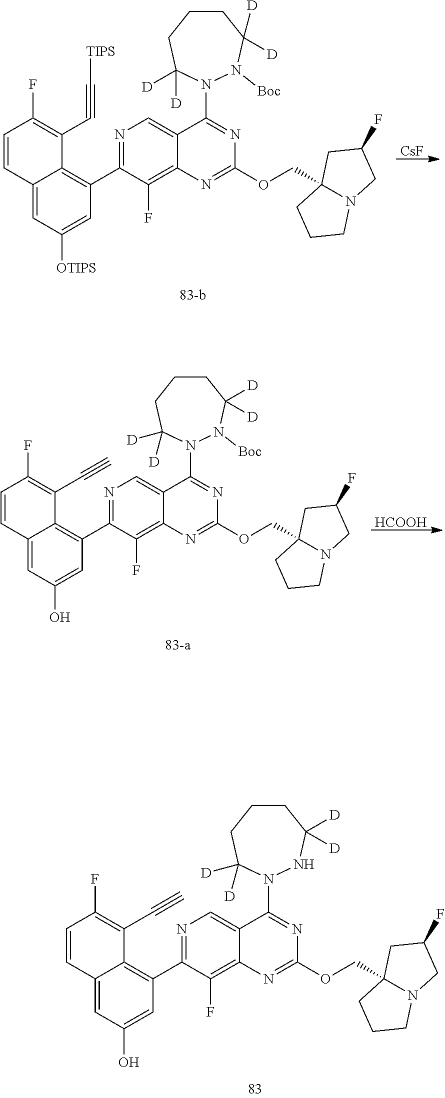

### Synthesis of Compound 83-h

To a reaction flask, diethyl glutarate (3000 mg, 15.94 mmol) and tetrahydrofuran (60 mL) were added. Placed in an ice-water bath for 10 min, the above mixture was added lithium aluminum deuteride (1200 mg, 28.59 mmol), then stirred at room temperature for 12 hours. The reaction mixture was quenched by adding solid sodium decahydrate sulfate until no bubbles were released, filtered, and the organic phase was concentrated to give compound 83-h (1320 mg, 77%) which was directly used in the next step. ¹H NMR (400 MHz, CDCl₃): δ 2.29 (s, 2H), 1.62-1.53 (m, 4H), 1.49-1.40 (m, 2H).

### Synthesis of Compound 83-g

To a reaction flask were added 83-h (1300 mg, 12.02 mmol), dichloromethane (40 mL), triethylamine (4864 mg, 48.07 mmol) and p-toluenesulfonyl chloride (6873 mg, 36.05 mmol), respectively. The reaction mixture was stirred overnight under protection of nitrogen, then was added water (20 mL). The aqueous phase was extracted with dichloromethane (50 mL*3). The organic phase was concentrated and the residue was purified by column chromatography (petroleum ether/ethyl acetate=1/1) to afford compound 83-g (4036 mg, 81%). LC-MS (ESI): m/z=434.7(M+NH₄)⁺.

### Synthesis of Compound 83-f

To a reaction flask were added 83-g (4000 mg, 9.60 mmol), acetonitrile (100 mL), 2-(tert-butyl)hydrazine-1,2-dicarboxylic acid-1-benzyl ester (2711 mg, 10.18 mmol) and cesium carbonate (15644 mg, 48.02 mmol). After degassed and purged with nitrogen for three times, the reaction flask was placed into an oil bath preheated at 65° C. and stirred for 36 hours. The reaction mixture was filtered. The organic phase was concentrated to dryness and the residue was purified by column chromatography (petroleum ether/ethyl acetate=10/3) to afford compound 83-f (2650 mg, 82%).

LC-MS (ESI): m/z=356.4(M+NH₄)⁺.

### Synthesis of Compound 83-e

To a reaction flask were added 83-f (2650 mg, 7.83 mmol), ethyl acetate (150 mL) and Pd/C (700 mg, 10%). After degassed and purged with hydrogen for three times, the reaction mixture was stirred at room temperature for 12 hours. The reaction mixture was filtered and the organic phase was concentrated to give compound 83-e (1400 mg, 88%). LC-MS (ESI): m/z=205.3 (M+H)⁺.

### Synthesis of Compound 83-d

A reaction vial charged with 83-e (700 mg, 3.43 mmol), dichloromethane (30 mL), DIPEA (1329 mg, 10.28 mmol), 2,4,7-trichloro-8-fluoropyrido[4,3-d]pyrimidine (865 mg, 3.43 mmol) was stirred for 12 hr at this temperature and protected by nitrogen. The reaction mixture was quenched with saturated sodium bicarbonate (100 mL), extracted with dichloromethane (100 mL*2). The organic phase was concentrated and the residue was purified by column chromatography (PE/EA=10/1) to give compound 83-d (1400 mg, 97%). LC-MS (ESI): m/z=420.3 (M+H)⁺.

### Synthesis of Compound 83-c

A reaction vial charged with 83-d (800 mg, 1.90 mmol), dichloromethane (30 mL), sodium tert-butoxide (366 mg, 3.81 mmol) and ((2R,7aS)-2-fluorohexahydro-1H-pyrrolozin-7a-yl)methanol (333 mg, 2.09 mmol) was stirred in an ice-water bath at atmosphere of nitrogen for 3 hr, then stirred at room temperature overnight. The reaction mixture was quenched by adding water (20 mL) and the aqueous phase was extracted with dichloromethane (50 mL*3). The organic phase was concentrated and the residue was purified by column chromatography (MeOH/DCM=1/10) to afford compound 83-c (890 mg, 86%). LC-MS (ESI): m/z=543.7(M+H)⁺.

### Synthesis of Compound 83-b

83-c (290 mg, 0.53 mmol), tetrahydrofuran (10 mL), water (1 mL), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (500 mg, 0.80 mmol), potassium phosphate (397 mg, 1.87 mmol) and cataCXium A Pd G3 (78 mg, 0.11 mmol) were combined in a reaction vial. After degassed and purged with nitrogen for three times, the flask was placed in an oil bath preheated at 65° C. and stirred for 12 hours. The reaction mixture was concentrated to dryness and the residue was purified by column chromatography (mobile phase: methanol/dichloromethane=0/100; 1/10) to afford compound 83-b (460 mg, 86%). LC-MS (ESI): m/z=1005.5(M+H)⁺.

### Synthesis of Compound 83-a

To a reaction flask were added 83-b (460 mg, 0.46 mmol), DMF (5 mL) and cesium fluoride (695 mg, 4.58 mmol). The reaction mixture was stirred at room temperature for 6 hours, then was added water (60 mL) and the aqueous phase was extracted with ethyl acetate (60 mL*3). The organic phase was concentrated and the residue was purified by column layer (MeOH/DCM=0/100, 1/10) to give compound 83-a (270 mg, 85%).

### Synthesis of Compound 83

A reaction vial charged with 83-a (200 mg, 0.29 mmol) and formic acid (10 mL) was stirred for 12 h at room temperature under protection of nitrogen. The reaction mixture was concentrated at room temperature and the residue was purified by prep-HPLC to afford compound 83 (50 mg, 29%), LC-MS (ESI): m/z 593.5(M+H)*.

### Synthetic Route of Compound 84

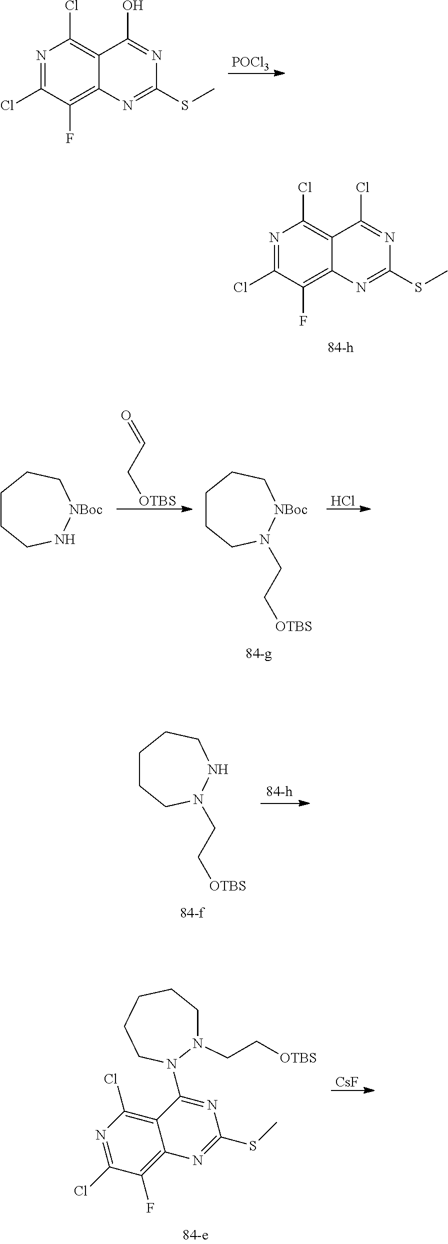

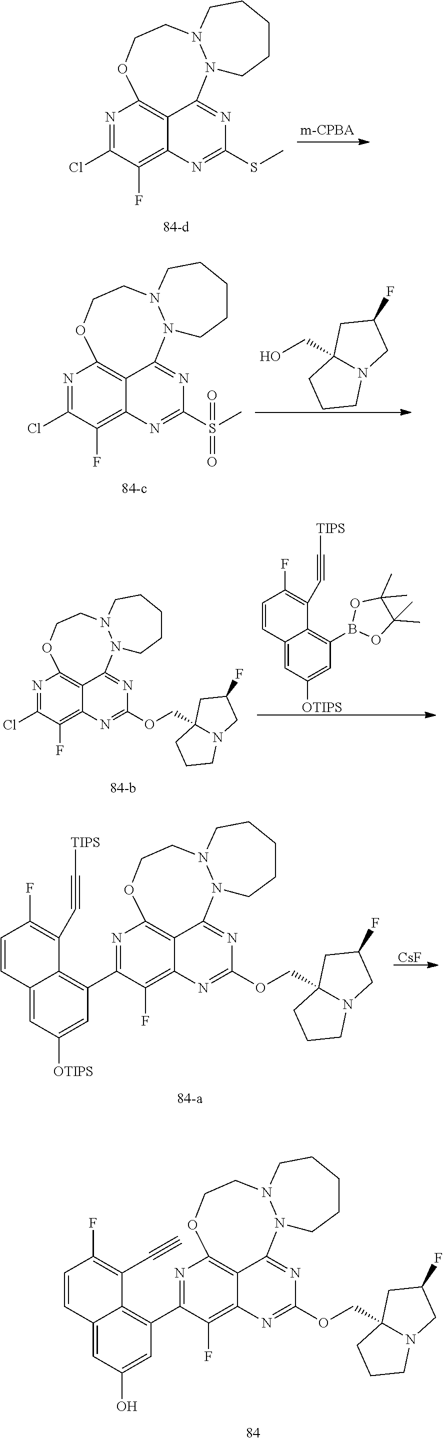

### Synthesis of Compound 84-h

At room temperature, to a reaction vial were added 5,7-dichloro-8-fluoro-2-(methylthio)pyrido[4,3-d]pyrimidin-4(3H)-one (2500 mg, 8.93 mmol), phosphorus oxychloride (10 mL), DIPEA (3461 mg, 26.78 mmol). The reaction vial was placed in an oil bath preheated at 85° C. and stirred for 3 hours. The reaction mixture was concentrated to dryness and the crude product was added ice-coldwater. The aqueous phase was extracted with dichloromethane (100 mL*3) and the organic phase was dried over sodium sulfate. Filtered and the organic phase was concentrated to give compound 84-h (1890 mg, 71%).

### Synthesis of Compound 84-g

At room temperature, to a reaction vial were added tert-butyl 1,2-diazepane-1-carboxylate (2730 mg, 13.63 mmol), 2-tert-butyldimethylsilyloxyacetaldehyde (4900 mg, 28.11 mmol) and methanol (80 mL). Placed in an ice-water bath, the reaction mixture was added sodium cyanoborohydride (4900 mg, 77.98 mmol) slowly, and stirred at room temperature for for 24 hours. The reaction mixture was concentrated, the residue was added water (100 mL) and the aqueous phase was extracted with ethyl acetate (100 mL*3). The organic phase was concentrated and the residue was purified by column chromatography (PE/EA=20/1) to afford compound 84-g (3910 mg, 80%). LC-MS (ESI): m/z=359.4(M+H)*.

### Synthesis of Compound 84-f

To a reaction flask were added 84-g (800 mg, 2.23 mmol) and acetonitrile (10 mL), respectively. After stirred under protection of nitrogen in an ice-water bath for 10 min, the reaction mixture was added hydrochloride/1,4-dioxane (4M, 1 mL, 4.00 mmol) dropwise and stirred in an ice-water bath for 4 hours. Placed in an ice-water bath, the reaction mixture was added ammonia methanol (2 mL, 14.00 mmol, 7 M). Concentrated and the residue was purified by column chromatography (MeOH/DCM=0/100, 1/10) to give compound 84-f (70 mg, 12%). LC-MS (ESI): m/z=259.3(M+H)⁺.

### Synthesis of Compound 84-e

To a reaction flask were added 84-h (196 mg, 0.66 mmol), dichloromethane (20 mL), DIPEA (255 mg, 1.97 mmol) and the mixture was stirred at −78° C. for 10 min, then was added 84-f (170 mg, 0.66 mmol) and stirred at −78° C. for half an hour, then stirred for 2 hours at room temperature. The reaction mixture was concentrated at room temperature and the residue was added saturated sodium bicarbonate solution (30 mL). The aqueous phase was extracted with dichloromethane (30 mL*2). The organic phase was concentrated and the residue was purified by column chromatography (PE/EA=10/1) to afford compound 84-e (249 mg, 73%). LC-MS (ESI): m/z=520.3 (M+H)⁺.

### Synthesis of Compound 84-d

A reaction flask charged with 84-e (249 mg, 0.48 mmol), DMF (5 mL) and cesium fluoride (220 mg, 1.45 mmol) was stirred at 55° C. for 3 hours under protection of nitrogen. The reaction mixture was added water (50 mL), extracted with ethyl acetate (50 mL*3), purified by column chromatography (MeOH/DCM=0/100, 1/10) to give compound 84-d (143 mg, 81%). LC-MS (ESI): m/z=370.2 (M+H)⁺.

### Synthesis of Compound 84-c

At room temperature, to a reaction vial were added 84-d (140 mg, 0.38 mmol), ethyl acetate (20 mL) and m-chloroperoxybenzoic acid (192 mg, 0.95 mmol, 85%). After stirred at 0° C. for 2 hrs, the reaction mixture was then stirred at room temperature for another 1 hr. The reaction mixture was added saturated sodium bicarbonate solution (100 mL) and the aqueous phase was extracted with ethyl acetate (50 mL*2). The organic phase was concentrated and the residue was purified by column chromatography (PE/EA=3/1, 0/100) to give compound 84-c (130 mg, 85%). LC-MS (ESI): m/z=402.1 (M+H)⁺.

### Synthesis of Compound 84-b

A reaction vial charged with ((2R,7aS)-2-fluorohexahydro-1H-pyrrolizin-7a-yl)methanol (68 mg, 0.43 mmol), 84-c (130 mg, 0.32 mmol), toluene (10 mL) and sodium tert-butoxide (50 mg, 0.52 mmol) was stirred for 3 h at room temperature under protection of nitrogen. The reaction mixture was mixed with silica gel and purified by column chromatography (MeOH/DCM=0/100, 1/10) to give compound 84-b (96 mg, 62%). LC-MS (ESI): m/z=481.3(M+H)⁺.

### Synthesis of Compound 84-a

A reaction vial charged with 84-b (96 mg, 0.20 mmol), tetrahydrofuran (6 mL), potassium phosphate (169 mg, 0.80 mmol), water (0.8 mL), cataCXium A Pd G3 (44 mg, 0.06 mmol) and ((6-fluoro-4-(4,4,5,5-tetramethyl-1,3,2-dioxaborolane-2-yl)-5-((triisopropylmethylsilyl)ethynyl)naphthalen-2-yl)oxy)triisopropylsilane (249 mg, 0.40 mmol) was degassed and purged with nitrogen for three times, then placed in an oil bath preheated at 65° C. and the reaction mixture was stirred for 12 hours. The reaction mixture was concentrated to dryness and the residue was purified by column chromatography (mobile phase: methanol/dichloromethane=0/100; 1/10) to give compound 84-a (110 mg, 58%).

### Synthesis of Compound 84

To a reaction flask were added 84-a (90 mg, 0.10 mmol), DMF (5 mL) and cesium fluoride (145 mg, 0.95 mmol). The reaction mixture was stirred at room temperature for 6 hours, then was added water (50 mL), extracted with ethyl acetate (50 mL*2), concentrated, and the residue was purified by prep-HPLC to obtain compound 84 (39 mg, 65%). LC-MS (ESI): m/z=631.3 (M+H)⁺; ¹H NMR (400 MHz, DMSO-d₆): δ 10.15 (1H, brs), 7.99-7.92 (1H, m), 7.49-7.42 (1H, m), 7.38-7.33 (1H, m), 7.26-7.04 (1H, m), 5.28 (1H, d, J=54.0 Hz), 4.60-3.93 (6H, m), 3.59-3.33 (3H, m), 3.18-2.95 (5H, m), 2.93-2.73 (2H, m), 2.25-1.93 (4H, m), 1.90-1.63 (7H, m).

### Synthetic Route of Compound 85

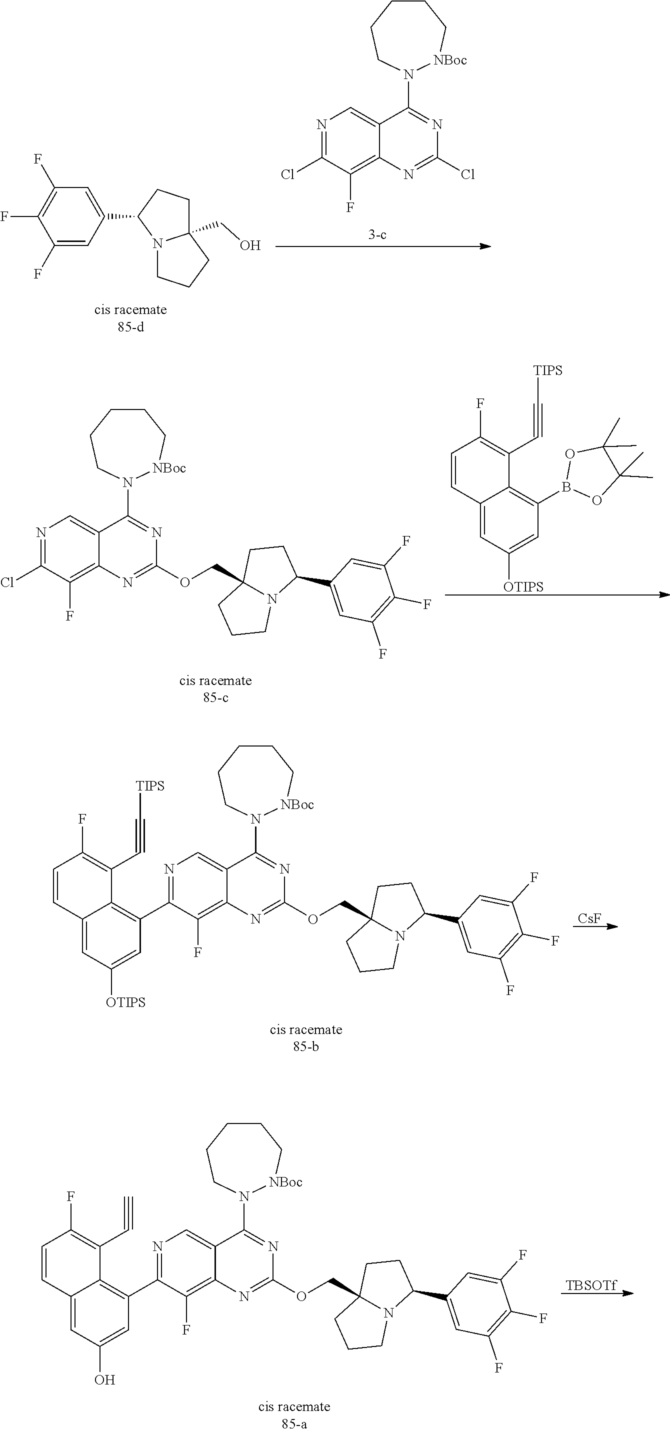

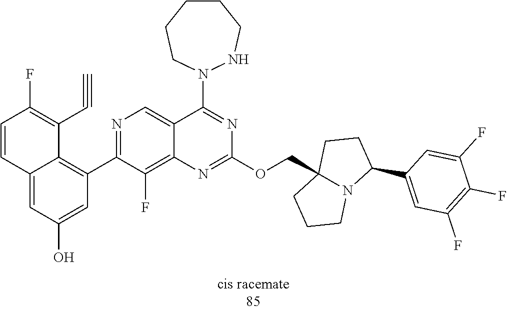 [SMILES: C#Cc1c(F)ccc2cc(O)cc(-c3ncc4c(N5CCCCCN5)nc(OC[C@]56CCCN5[C@H](c5cc(F)c(F)c(F)c5)CC6)nc4c3F)c12]

Compound 85-d was synthesized according to the method of Patent CN116891488A.

### Synthesis of Compound 85-c

85-d (170 mg, 0.63 mmol), dichloromethane (20 mL), 3-c (391 mg, 0.94 mmol) and sodium tert-butoxide (121 mg, 1.25 mmol) were added to a reaction flask at room temperature. After stirred for 3 hours at 0° C. under protection of nitrogen, the reaction mixture was added saturated ammonium chloride (100 mL) to and the aqueous phase was extracted with dichloromethane (50 mL*2). The organic phase was concentrated and the residue was purified by column chromatography (PE/EA=10/1, 3/1) to give compound 85-c (290 mg, 71%).

### Synthesis of Compound 85-b

To a microwave tube were added 85-c (170 mg, 0.26 mmol), tetrahydrofuran (6 mL), water (0.8 mL), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (326 mg, 0.52 mmol), potassium phosphate (166 mg, 0.78 mmol) and cataCXium A Pd G3 (57 mg, 0.08 mmol). After degassed and purged with nitrogen for three times, the microwave tube was placed in an oil bath preheated at 65° C. and stirred for 8 hours. The reaction mixture was concentrated and the residue was purified by column chromatography (PE/EA=5/1, 1/1) to afford compound 85-b (226 mg, 78%). LC-MS (ESI): m/z=1113.4(M+H)⁺.

### Synthesis of Compound 85-a

To a reaction flask were added 85-b (230 mg, 0.21 mmol), DMF (5 mL) and cesium fluoride (1300 mg, 8.56 mmol). The reaction mixture was stirred at room temperature under protection of nitrogen for 10 h, then was added ammonium chloride (100 mL) and the aqueous phase was extracted with ethyl acetate (100 mL*3). The organic phase was concentrated and the residue was purified by column chromatography (PE/EA=100/0, 3/1) to afford compound 85-a (133 mg, 80%). LC-MS (ESI): m/z=801.4(M+H)*.

### Synthesis of Compound 85

To a reaction flask were added 85-a (130 mg, 0.16 mmol) and dichloromethane (6 mL). After stirred in an ice-water bath for 10 min, the reaction mixture was added TBSOTf (20 mg, 0.08 mmol) slowly and stirred in an ice-water bath for 1 hr. The reaction mixture was added saturated aqueous sodium bicarbonate (50 mL), stirred at room temperature for 17 h. The reaction mixture was extracted with dichloromethane (20 mL*3). The organic phase was concentrated and the residue was purified by prep-HPLC to afford compound 85 (68 mg, 60%). LC-MS (ESI): m/z 701.1(M+H)⁺.

### Synthetic Route of Compound 86

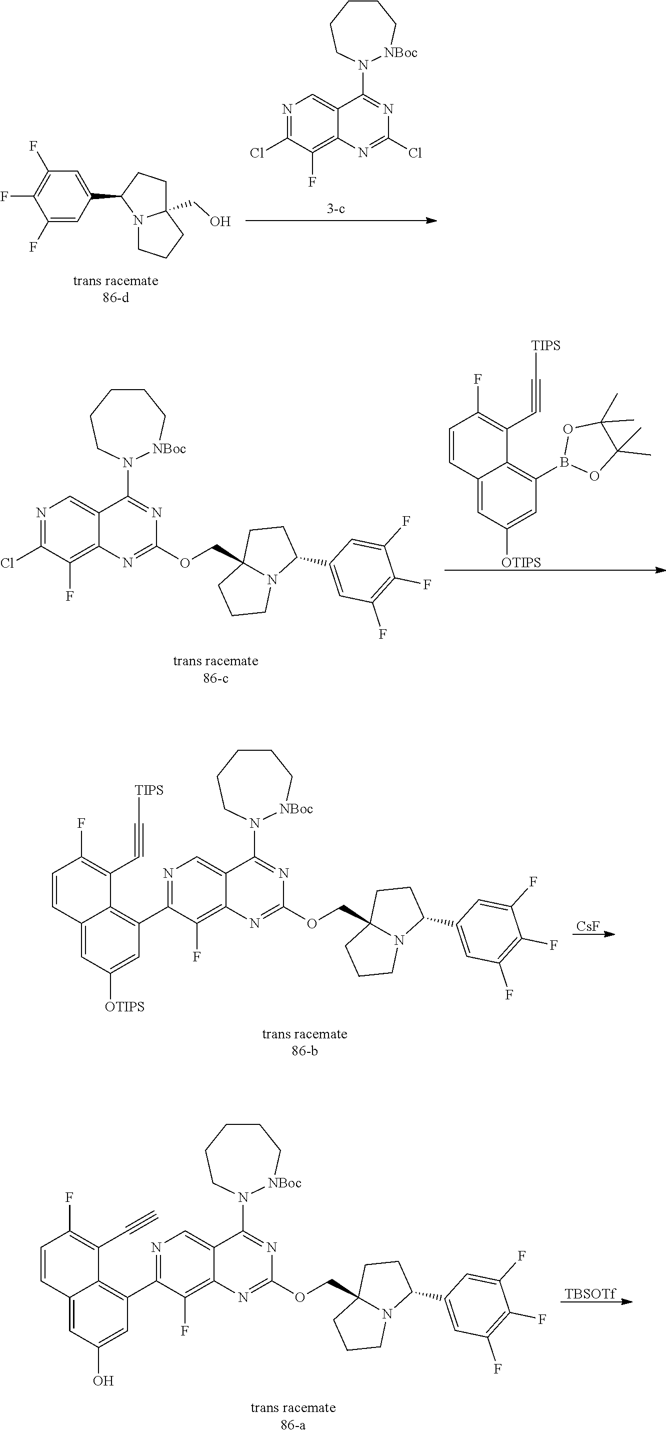

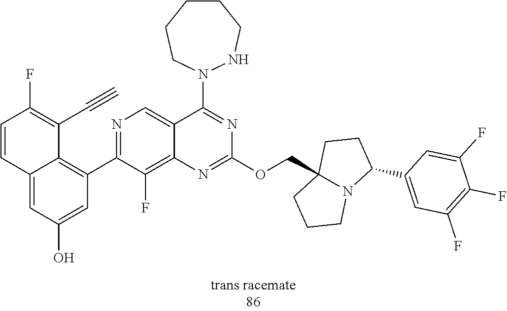 [SMILES: C#Cc1c(F)ccc2cc(O)cc(-c3ncc4c(N5CCCCCN5)nc(OC[C@]56CCCN5[C@@H](c5cc(F)c(F)c(F)c5)CC6)nc4c3F)c12]

### Synthesis of Compound 86-d

Synthesized according to the method of Patent CN116891488A.

### Synthesis of Compound 86-c

86-d (120 mg, 0.44 mmol), dichloromethane (20 mL), 3-c (276 mg, 0.66 mmol) and sodium tert-butoxide (164 mg, 0.66 mmol) were added to a reaction flask at room temperature. After stirred for 3 hours at 0° C. under protection of nitrogen, the reaction mixture was added saturated ammonium chloride (100 mL) and the aqueous phase was extracted with dichloromethane (50 mL*2). The organic phase was concentrated and the residue was purified by column chromatography (DCM/MeOH=100/0, 20/1) to afford compound 86-c (159 mg, 55%). LC-MS (ESI): m/z=651.1(M+H)⁺.

### Synthesis of Compound 86-b

To a microwave tube were added 86-c (159 mg, 0.24 mmol), tetrahydrofuran (6 mL), water (0.8 mL), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (305 mg, 0.49 mmol), potassium phosphate (156 mg, 0.73 mmol) and cataCXium A Pd G3 (53 mg, 0.07 mmol). After degassed and purged with nitrogen for three times, it was placed in an oil bath preheated at 65° C. and stirred for 8 hours. The reaction mixture was concentrated and the residue was purified by column chromatography (DCM/MeOH=100/0, 20/1) to afford compound 86-b (196 mg, 72%).

LC-MS (ESI): m/z=1113.4(M+H)⁺.

### Synthesis of Compound 86-a

To a reaction flask were added 86-b (196 mg, 0.18 mmol), DMF (5 mL) and cesium fluoride (1300 mg, 8.56 mmol). After stirred at room temperature under protection of nitrogen for 10 h, the reaction mixture was added ammonium chloride (100 mL) and the aqueous phase was extracted with ethyl acetate (100 mL*3). The organic phase was concentrated and the residue was purified by column chromatography (DCM/MeOH=100/0, 10/1) to afford compound 86-a (134 mg, 95%). LC-MS (ESI): m/z=801.6(M+H)⁺.

### Synthesis of Compound 86

To a reaction flask were added 86-a (90 mg, 0.11 mmol) and dichloromethane (6 mL). After stirred in an ice-water bath for 10 min, the reaction mixture was added TBSOTf (20 mg, 0.08 mmol) slowly and stirred in an ice-water bath for 1 hr. The reaction mixture was added saturated aqueous sodium bicarbonate (50 mL), stirred at room temperature for 17 h. The reaction mixture was extracted with dichloromethane (20 mL*3). The organic phase was concentrated and the residue was purified by prep-HPLC to give compound 86 (40 mg, 51%), LC-MS (ESI): m/z 701.3(M+H)⁺.

### Synthetic Route of Compound 87

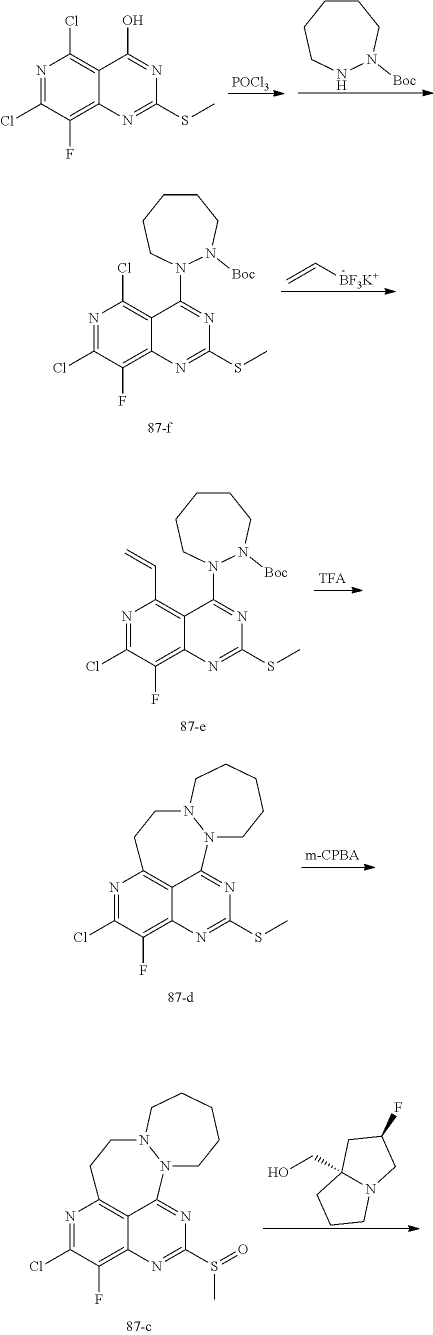

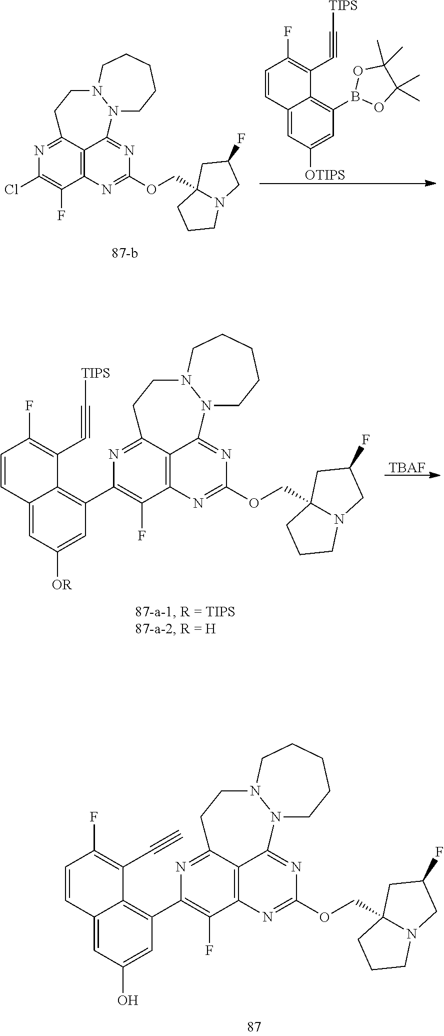

### Synthesis of Compound 87-f

Phosphorus oxychloride (1.31 g, 8.57 mmol) and N,N - diisopropylethylamine (3.0 mL, 17.14 mmol) were added to a reaction vial charged with 5,7-dichloro-8-fluoro-2-(methylthio)pyrido[4,3-d]pyrimidin-4(1H)-one (2 g, 7.14 mmol) and acetonitrile (30 mL) under the protection of nitrogen. The reaction mixture was stirred at 85° C. for 1 hour, then was cooled to −10° C., was added a solution of 1,2-diazepane-1-carboxylic acid tert-butyl ester (1.43 g, 7.14 mmol)in acetonitrile solution (10 mL) and N,N-diisopropylethylamine (0.55 g, 4.28 mmol), then stirred for 1 h at room temperature and concentrated to dryness. The residue was added water (30 mL), stirred for 15 min, diluted with ethyl acetate (30 mL), and the aqueous phase was then extracted with ethyl acetate (20 mL). The organic phases were combined, washed with brine(30 mL), dried over anhydrous sodium sulfate, and the residue was purified by column chromatography (mobile phase: ethyl acetate/petroleum ether=0-20%) to give compound 87-f (2.89 g, 87%). LC-MS (ESI): m/z 461.9 (M+H)⁺.

### Synthesis of Compound 87-e

A reaction vial charged with 87-f (500 mg, 1.08 mmol), potassium vinyl trifluoroborate (188 mg, 1.41 mmol), sodium bicarbonate (218 mg, 2.60 mmol), tetrakis(triphenylphosphine)palladium (125 mg, 0.11 mmol) and ethanol (5 mL) was degassed and purged with nitrogen for three times, stirred at 80° C. for 5 hours. Dichloromethane (20 mL) and water (20 mL) were added to the reaction mixture. Partitioned, the aqueous phase was extracted with dichloromethane (20 mL). The organic phases were combined, dried over anhydrous sodium sulfate, filtered, and the filtrate was concentrated to dryness and the residue was purified by column chromatography (mobile phase: ethyl acetate: petroleum ether=0-80%) to give compound 87-e (210 mg). LC-MS (ESI): m/z 454.4 (M+H)⁺.

### Synthesis of Compound 87-d

87-e (210 mg, 0.19 mmol, 40% purity), trifluoroacetic acid (2 mL) and dichloromethane (2 mL) were added to a reaction vial and the reaction mixture was stirred at room temperature for 1 hour. Removed the organic solvent by rotary evaporation. The residue was added dichloromethane (10 mL) and water (10 mL), neutralized to pH 8-9 with saturated sodium bicarbonate. Partitioned, the aqueous phase was extracted with dichloromethane (10 mL). The organic phases were combined, dried over anhydrous sodium sulfate, filtered, the filtrate was concentrated to dryness, and the residue was purified by column chromatography (mobile phase: ethyl acetate: petroleum ether=0-80%) to give compound 87-d (50 mg, 13% in two-step reaction). LC-MS (ESI): m/z 354.1 (M+H)⁺.

### Synthesis of Compound 87-c

To a reaction flask charged with 87-d (240 mg, 0.68 mmol) and dichloromethane (2 mL) was added m-chloroperoxybenzoic acid (151 mg, 0.75 mmol) in an ice-water bath. The reaction mixture was stirred at 0-5° C. for 1 h, then was added saturated sodium bicarbonate to pH 9-10, then dichloromethane (10 mL). Partitioned, the aqueous phase was extracted with dichloromethane (10 mL). The organic phases were combined, washed with brine(10 mL), dried over anhydrous sodium sulfate, filtered, and the filtrate was concentrated to dryness to give compound 87-c (260 mg, 100%).

LC-MS (ESI): m/z 370.1 (M+H)⁺.

### Synthesis of Compound 87-b

A solution of ((2R,7aS)-2-fluorohexahydro-1H-pyrrolylazin-7a-yl)methanol (124 mg, 0.78 mmol) in anhydrous tetrahydrofuran (2.5 mL) was added into a solution of sodium tert-butoxide (81 mg, 0.85 mmol) in anhydrous toluene (1 mL) dropwise at -10 to −5° C. The mixture was stirred at −10° C. for 30 min, then was added a solution of 87-c (250 mg, 0.68 mmol) in anhydrous tetrahydrofuran solution (10 mL) dropwise, and stirred at -10 to −5° C. for 1 hour. It was quenched by addition of brine (5 mL), stirred for 10 min and extracted twice with ethyl acetate (10 mL). The organic phases were combined, filtered, and the filtrate was concentrated to dryness and the residue was purified by column chromatography (mobile phase: methanol: dichloromethane=0-5%) to give compound 87-b (153 mg). LC-MS (ESI): m/z 464.9 (M+H)⁺.

### Synthesis of Compounds 87-a-1 and 87-a-2

To a microwave tube were added 87-b (60 mg, 0.13 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (97 mg, 0.16 mmol), methanesulfonated [butylbis(1-adamantyl)phosphine](2-amino-1,1′-biphenyl-2-yl)palladium(II) (14 mg, 0.02 mmol), potassium phosphate (82 mg, 0.39 mmol), tetrahydrofuran (2.0 mL) and water (0.4 mL). The reaction mixture was sealed, degassed and purged with nitrogen for three times and microwaved at 90° C. for 6 hours. The reaction mixture was extracted twice with dichloromethane (5 mL). The organic phases were combined, filtered, and the filtrate was concentrated to dryness. The residue was purified by column chromatography (mobile phase: ethyl acetate: petroleum ether=10-100%; methanol: dichloromethane=0-8%) to obtain 87-a-1 (45 mg) and 87-a-2 deprotected of TIPS (89 mg, 42% purity). 87-a-1: LC-MS (ESI): m/z 927.3 (M+H)⁺. 87-a-2: LC-MS (ESI): m/z 771.2 (M+H)⁺.

### Synthesis of Compound 87

To a solution of 87-a-1 (45 mg, 0.05 mmol) in tetrahydrofuran (2.4 mL) was added tetrabutylammonium fluoride trihydrate (39 mg, 0.13 mmol). The resulting mixture was stirred at room temperature for 2 hours. To a solution of 87-a-2 (89 mg, 42% purity, 0.05 mmol) in tetrahydrofuran (2 mL) was added tetrabutylammonium fluoride trihydrate (39 mg, 0.13 mmol) and the mixture was stirred at room temperature for 2 hours. The reaction mixtures were combined, was added dichloromethane (10 mL) and water (10 mL). Partitioned, the aqueous phase was extracted with dichloromethane (10 mL). The organic phases were combined, anhydrous sodium sulfate was dried, filtered, concentrated to dryness, and the residue was purified by column chromatography (mobile phase: methanol: dichloromethane=0-8%), concentrated, lyophilized to give compound 87 (43 mg). LC-MS (ESI): m/z 615.7 (M+H)⁺.

### Synthetic Route of Compound 88

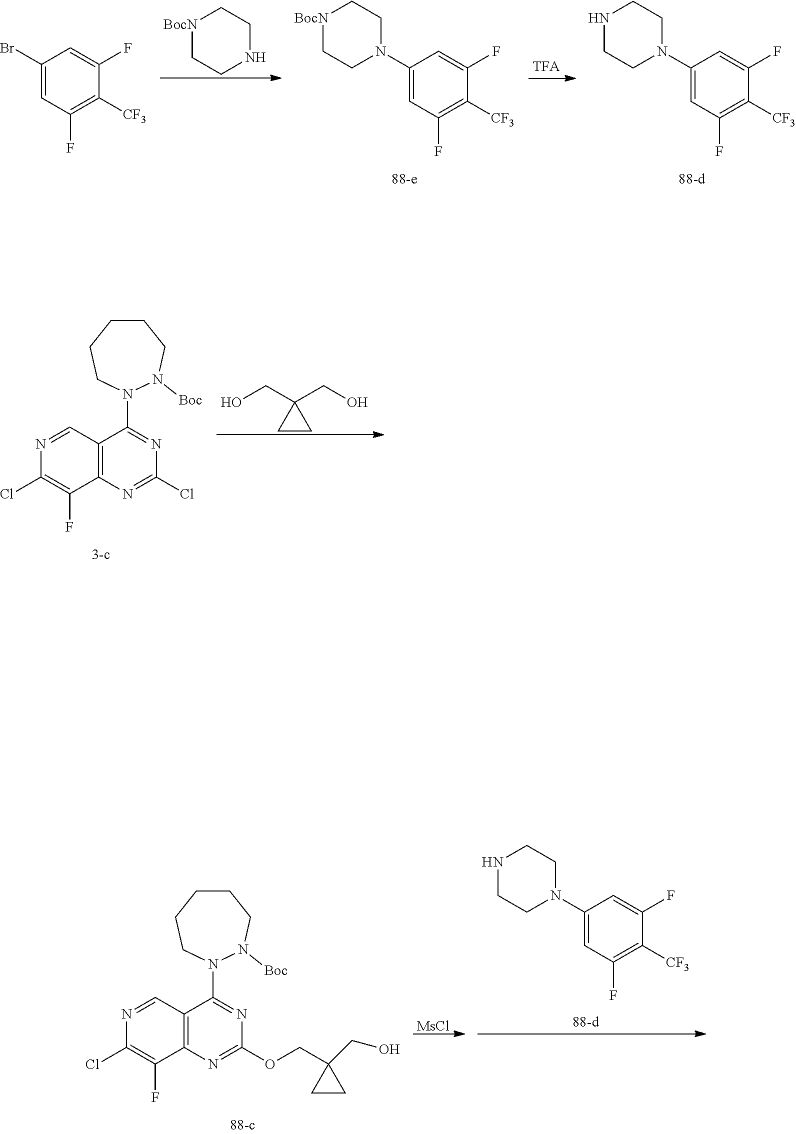

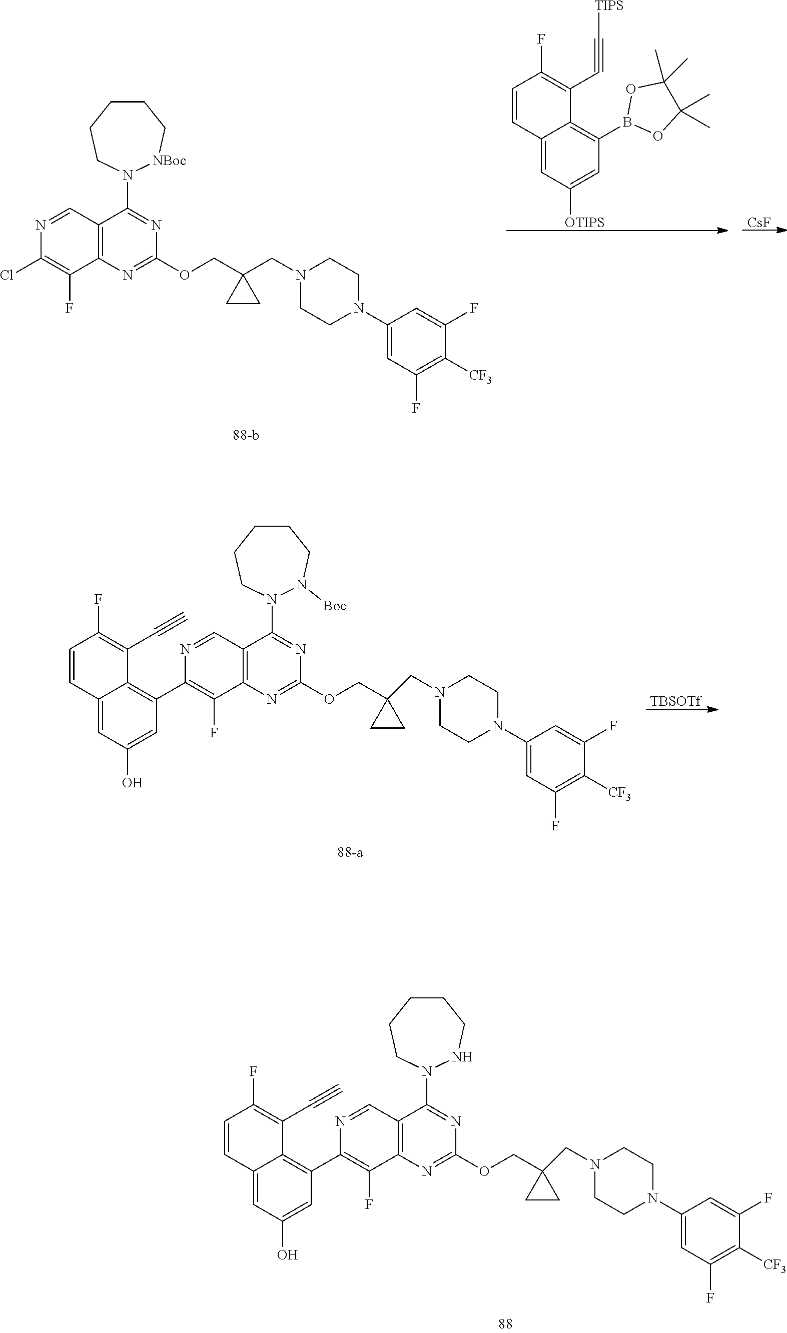

### Synthesis of Compound 88-e

A reaction vial were added N-Boc-piperazine (2.23 g, 11.96 mmol), 3,5-difluoro-4-trifluoromethylbromobenzene (2.23 g, 8.54 mmol), tris(dibenzylideneacetone)dipalladium (780 mg, 0.85 mmol), 1,3-bis(2,6-diisopropylphenyl)imidazole chloride (730 mg, 1.71 mmol), sodium tert-butoxide (1.23 g, 12.81 mmol) and toluene (22 mL). After degassed and purged with nitrogen for three times, the reaction mixture was stirred at 100° C. for 2 hours. Cooled to room temperature, the reaction mixture was added water (50 mL), ethyl acetate (50 mL). Partitioned, and the aqueous phase was extracted with ethyl acetate (20 mL). The organic phases were combined, dried over anhydrous sodium sulfate, filtered, the filtrate was concentrated to dryness, and the residue was purified by column chromatography (mobile phase: ethyl acetate: petroleum ether=0-10%) to give compound 88-e (2.30 g, 73%). LC-MS (ESI): m/z 367.1 (M+H)⁺.

### Synthesis of Compound 88-d

To a reaction vial were added 88-e (3.00 g, 8.19 mmol), dichloromethane (30 mL) and trifluoroacetic acid (14.00 g, 123 mmol), and the above mixture was stirred at room temperature for 3 h. The reaction mixture was concentrated and then was added dichloromethane (50 mL), and was adjusted pH to 9-10 with saturated aqueous sodium carbonate, and the organic phase was concentrated to give compound 88-d (2.00 g 92%). LC-MS(ESI): m/z 267.1 (M+H)⁺.

### Synthesis of Compound 88-c

To a reaction flask were added 3-c (2 g, 4.80 mmol) and 1,1-cyclopropanedimethanol (2.14 g, 20.95 mmol), N,N-dimethylformamide (10 mL) and tetrahydrofuran (10 mL). After warmed up to 50° C., the above mixture was added cesium carbonate (5.4 g, 16.57 mmol) and DABCO (0.054 g, 0.48 mmol), and stirred at 50° C. for 1 hour. The reaction mixture was cooled to room temperature, was added water (100 mL). A large amount of solid was precipitated, and pulped at room temperature for 30 min, filtered, and the filter cake was dried to obtain 88-c (2.00 g, 86%). LCMS (ESI): m/z 482.1 (M+H)⁺.

### Synthesis of Compound 88-b

To a reaction flask in an ice-water bath was added 88-c (800 mg, 1.66 mmol), dichloromethane (10 mL), triethylamine (504 mg, 4.98 mmol), followed by dropwise addition of methylsulfonyl chloride (190 mg, 1.66 mmol) in dichloromethane (2 mL), and the reaction mixture was stirred in ice-water bath for half an hour. The reaction mixture was added water (30 mL), extracted with dichloromethane (30 mL), and the organic phase was dried and concentrated to give compound a clear oil. To the oil was added potassium iodide (827 mg, 4.98 mmol), triethylamine (504 mg, 4.98 mmol), 88-d (442 mg, 1.66 mmol), N,N-dimethylformamide (10 mL), and the mixture was stirred at 90° C. for 2 hours. Water (40 mL) was added to the reaction mixture and the aqueous phase was extracted twice with ethyl acetate (20 mL). The organic phases were combined, washed with brine (20 mL), dried over anhydrous sodium sulfate, concentrated, and the residue was purified by column chromatography (mobile phase: ethyl acetate: petroleum ether=0-20%) to give compound 88-b (600 mg, 50%). LCMS(ESI):m/z 730.5 (M+H)⁺.

### Synthesis of Compound 88-a

A reaction vial were added 88-b (400 mg, 0.55 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (342 mg, 0.55 mmol), methanesulfonic acid [n-butylbis(1-adamantyl ) phosphine](2-amino-1,1 ′-biphenyl-2-yl)palladium(II) (40 mg, 0.06 mmol), potassium phosphate (349 mg, 1.64 mmol), tetrahydrofuran (10 mL) and water (2 mL). After degassed and purged with nitrogen, the reaction mixture was stirred at 60° C. for 2.5 hours. Water (20 mL) was added to the reaction mixture and the aqueous phase was extracted with ethyl acetate (30 mL). The organic phase was concentrated to give a black crude compound (550 mg). The black crude was dissolved directly in N,N-dimethylformamide (10 mL), was added CsF (1.00 g, 6.58 mmol) and stirred at room temperature for 2 hours. To the reaction mixture, water (20 mL) was added and the aqueous phase was extracted twice with ethyl acetate (20 mL). The organic phases were combined, washed with saturated sodium chloride (20 mL), dried over anhydrous sodium sulfate, concentrated, and the residue was purified by column chromatography (mobile phase: ethyl acetate: petroleum ether=0-60%) to give compound 88-a (75 mg, 16%). LCMS (ESI): m/z 880.3 (M+H)⁺.

### Synthesis of Compound 88

Tert-butyldimethylsilyl trifluoromethanesulfonate (270 mg, 1.02 mmol) was added dropwise to a solution of 88-a (60 mg, 0.07 mmol) in dichloromethane (1.6 mL) in an ice-water bath, and the mixture was stirred for 40 min at 0-5° C., then was added saturated aqueous sodium bicarbonate (4 mL) and stirred at room temperature overnight. The mixture was extracted with dichloromethane/methanol (10:1) (10 mL*2). The organic phase was dried over anhydrous sodium sulfate, filtered, and the filtrate was concentrated to dryness purified over column (mobile phase: ethyl acetate: petroleum ether=10-80%) to obtain the crude product (40 mg), purified by prep-HPLC (mobile phase Acetonitrile: 10 mM aqueous ammonium bicarbonate=10-75%), concentrated, lyophilized to obtain compound 88 (28 mg, 53%). LC-MS (ESI): m/z 780.4 (M+H)⁺; ¹H NMR (400 MHz, CDCl₃) δ 10.16 (s, 1H), 7.51-7.41 (m, 1H), 7.25-7.18 (m, 1H), 7.09 (t, J=8.8 Hz, 1H), 7.00 (s, 1H), 6.28 (d, J=13.5 Hz, 2H), 4.47 (d, J=10.8 Hz, 1H), 4.33-4.20 (m, 2H), 4.10-3.96 (m, 2H), 3.32-2.94 (m, 7H), 2.85-2.29 (m, 7H), 1.98-1.88 (m, 2H), 1.78-1.60 (m, 4H), 0.79-0.63 (m, 2H), 0.61-0.39 (m, 2H).

### Synthetic Route of Compound 89

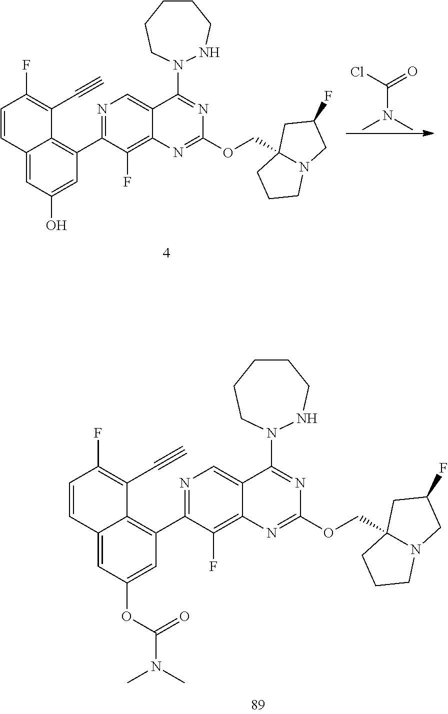

### Synthesis of Compound 89

Compound 4 (1 g, 1.70 mmol), DMF (20 mL) and potassium carbonate (704 mg, 5.10 mmol) were added to a reaction flask at room temperature, and the above mixture was added dimethylcarbamoyl chloride (235 uL, 2.55 mmol) dropwise while stirring. After the drop was completed, the reaction mixture was stirred at room temperature under protection of nitrogen for 2 h. The reaction mixture was quenched with water and the aqueous phase was extracted twice with ethyl acetate. The organic phase was washed three times with brine, dried over anhydrous sodium sulfate, concentrated to dryness by rotary evaporation, and the residue was purified by column chromatography (mobile phase: MeOH/DCM, 0-10%) to give compound 89 (990 mg, 88%). LC-MS (ESI): m/z 660.8 (M+H)⁺;¹H NMR (400M, DMSO-d₆): δ 10.27 (1H, s), 8.24-8.14 (1H, m), 7.97(1H, d, J=2.0 Hz), 7.63 (1H, t, J=8.8 Hz), 7.48 (1H, d, J=2.4 Hz), 5.89(1H, t, J=6.0 Hz), 5.31(1H, d, J=54.0 Hz), 4.26-3.88 (5H, m), 3.19-2.98 (8H, m), 2.94 (3H, s), 2.89-2.79 (1H, m), 2.20-1.56 (12H, m).

### Synthesis of Compound 90

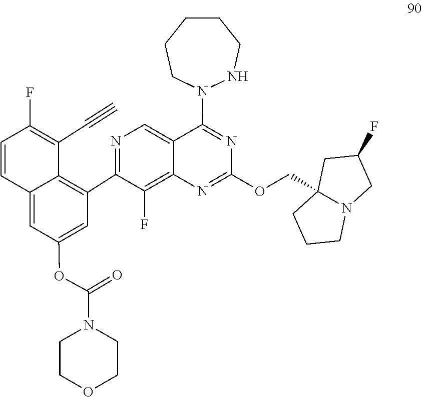 [SMILES: C#Cc1c(F)ccc2cc(OC(=O)N3CCOCC3)cc(-c3ncc4c(N5CCCCCN5)nc(OC[C@@]56CCCN5C[C@H](F)C6)nc4c3F)c12]

Referring to the synthetic route of compound 89, compound 90 was obtained using 4-morpholine carbonyl chloride instead of dimethylcarbamoyl chloride. LC-MS (ESI): m/z 702.8 (M+H)⁺.

### Synthetic Route of Compound 91

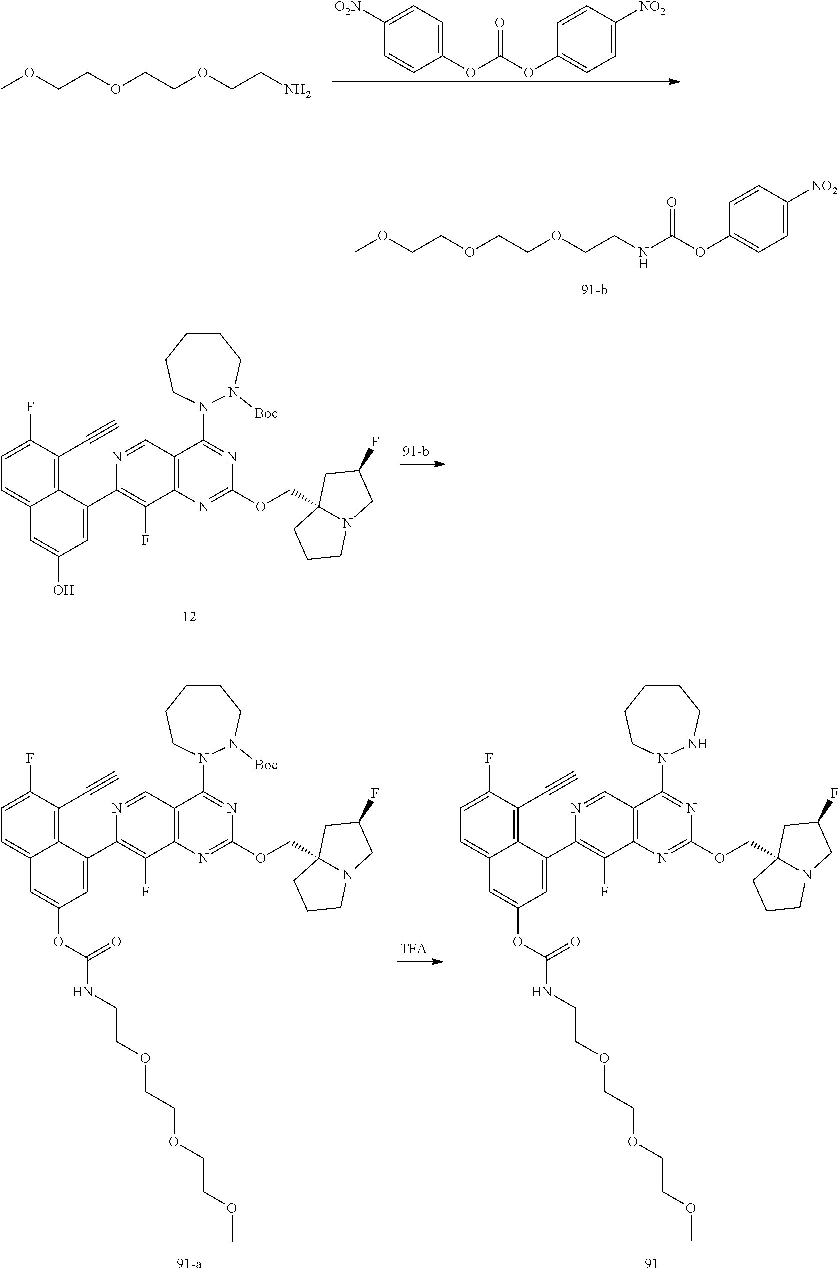

### Synthesis of Compound 91-b

Compound 2-(2-(2-methoxyethoxy)ethoxy)ethanamine (1.00 g, 6.13 mmol) and DMAP (1.5 g, 12.25 mmol) were dissolved in 25 mL of anhydrous acetonitrile at room temperature and then the mixture was added dropwise to a solution of bis(p-nitrophenyl) carbonate (1.96 g, 6.43 mmol) in anhydrous acetonitrile solution (25 mL) in an ice-water bath. The reaction mixture was stirred under protection of nitrogen at 0° C. for 30 minutes, then at room temperature for 1 hour. The reaction mixture was concentrated at reduced pressure and the residue was purified by flash column chromatography (EA/PE=0-100%) to give compound 91-b (1.6 g, 80%). LC-MS (ESI): m/z=329.1[M+H]⁺.

### Synthesis of Compound 91-a

Compound 91-b (381 mg, 1.16 mmol) was dissolved in 40 mL of anhydrous acetonitrile at room temperature, followed by addition of compound 12 (200 mg, 0.29 mmol) and DMAP (160 mg, 1.31 mmol) and the reaction mixture was stirred under protection of nitrogen at 50° C. overnight. The reaction mixture was concentrated at reduced pressure and the residue was dissolved in 100 mL of ethyl acetate, washed sequentially with water (100 mL), 10% NaHSO₄ (100 mL), saturated sodium bicarbonate solution (100 mL), brine (100 mL), dried over anhydrous sodium sulfate and filtered. The filtrate was concentrated at reduced pressure and the residue was purified by a flash column chromatography (MeOH/DCM=0-10%) to afford compound 91-a (150 mg, 59%). LC-MS (ESI): m/z=878.5[M+H]⁺.

### Synthesis of Compound 91

Compound 91-a (150 mg, 0.17 mmol) was dissolved in 6 mL of anhydrous DCM at room temperature, then TFA (2 mL) was added and the reaction mixture was stirred under protection of nitrogen for 4 h at room temperature. The reaction mixture was concentrated at reduced pressure and the residue was suspended in saturated sodium bicarbonate solution (100 mL) and the aqueous phase was extracted with DCM (100 mL). The organic phase was dried over anhydrous sodium sulfate, filtered, and the filtrate was concentrated at reduced pressure, and the residue was purified by Prep-TLC (MeOH/DCM=1:10) to afford compound 91 (108 mg, 82%). LC-MS (ESI): m/z=778.3[M+H]⁺.

### Synthetic Route of Compound 92

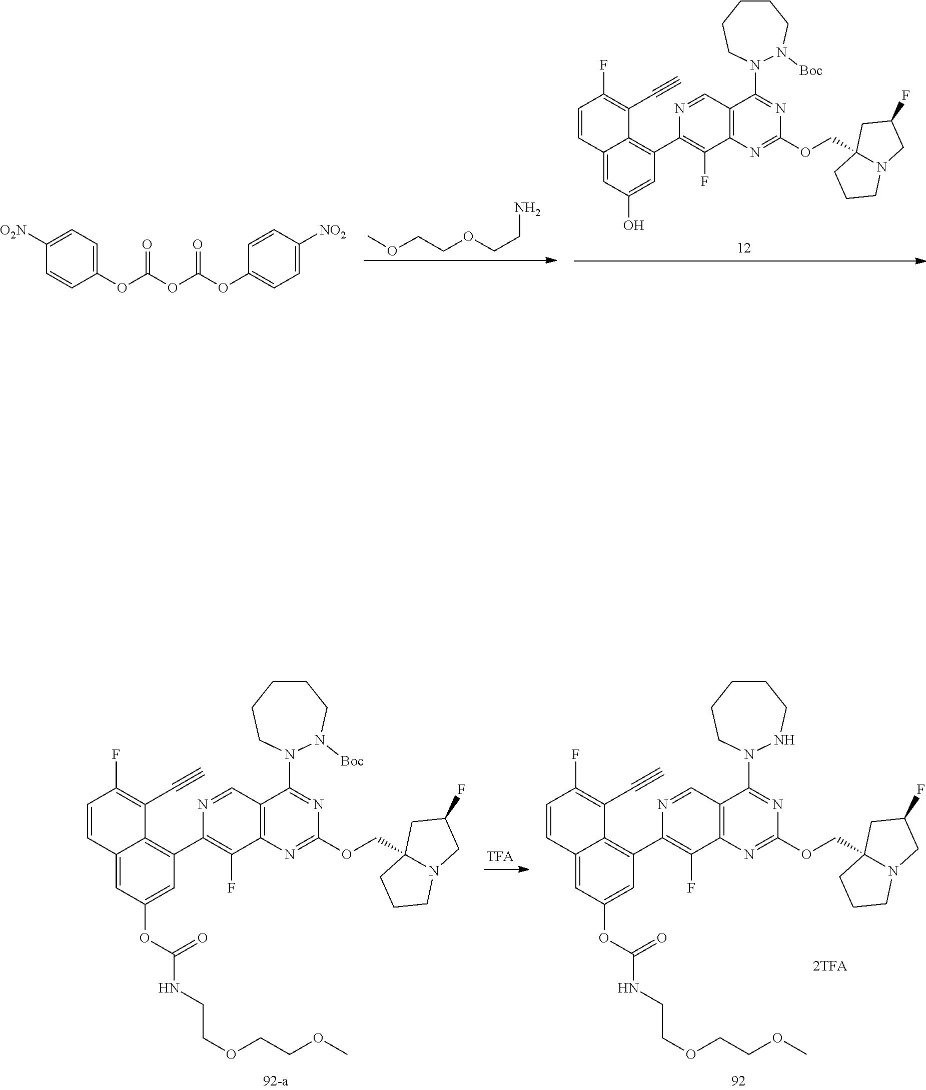

### Synthesis of Compound 92-a

To a solution of bis(p-nitrophenyl) carbonate (766 mg, 2.52 mmol) in acetonitrile (20 mL) was added 1-(2-aminoethoxy)-2-methoxyethane (300 mg, 2.52 mmol) and 4-dimethylaminopyridine (615 mg, 5.04 mmol) in acetonitrile (20 mL) dropwise at room temperature. After heated to 50° C., the reaction was allowed to proceed for 1 hour, then was added compound 12 (100 mg, 0.15 mmol) and stirred at 50° C. under protection of nitrogen overnight. The next day, removed acetonitrile by rotary evaporation, added 10% NaHSO₄ solution and a small amount of anhydrous sodium sulfate and extracted five times with ethyl acetate. The organic phase was dried, concentrated to dryness by rotary evaporation, dissolved in a small amount of dichloromethane, and the residue was purified by column chromatography (mobile phase: methanol/dichloromethane, 0-10%) to give compound 92-a (111 mg, 92%). LC-MS (ESI): m/z 834.0(M+H)⁺.

### Synthesis of Compound 92

Trifluoroacetic acid (1 mL) was added to a solution of 92-a (111 mg, 0.13 mmol) in dichloromethane (6 mL) at room temperature and stirred at room temperature under protection of nitrogen overnight. The next day, the reaction mixture was concentrated to dryness by rotary evaporation at room temperature, dissolved in acetonitrile, and purified by prep-HPLC (trifluoroacetic acid) to give 92 (56 mg, 64%) (trifluoroacetate salt). LC-MS (ESI): m/z734.6 (M+H)⁺.

### Synthetic Route of Compound 93

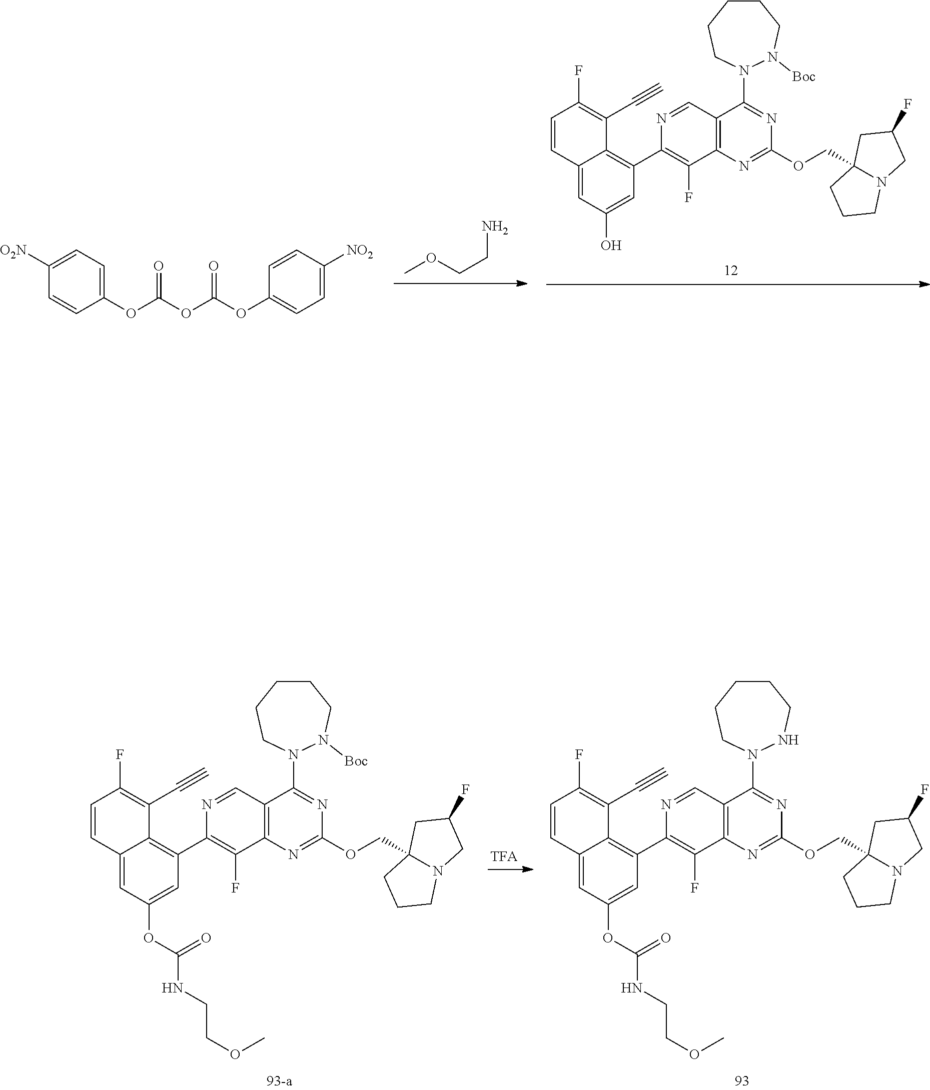

### Synthesis of Compound 93-a

To a solution of bis(p-nitrophenyl) carbonate (1215 mg, 3.99 mmol) in acetonitrile (20 mL) was added 2-methoxyethylamine (300 mg, 3.99 mmol) and 4-dimethylaminopyridine (976 mg, 7.99 mmol) in acetonitrile (20 mL) dropwise at room temperature. After heated to 50° C., the reaction mixture was stirred for 1 h. The reaction mixture was added compound 12 (275 mg, 0.40 mmol) and stirred at 50° C. under protection of nitrogen overnight. The next day, the reaction mixture was concentrated to dryness by rotary evaporation acetonitrile, added 10% NaHSO₄ solution and a small amount of anhydrous sodium sulfate, extracted with ethyl acetate for 3 times. The organic phase was dried, concentrated to dryness by rotary evaporation, and the residue was dissolved in a small amount of dichloromethane and purified by column chromatography (mobile phase: methanol/dichloromethane, 0-10%) to give 93-a (220 mg, 70%). LC-MS (ESI): m/z 790.9(M+H)⁺.

### Synthesis of Compound 93

Trifluoroacetic acid (2 mL) was added to a solution of 93-a (220 mg, 0.28 mmol) in dichloromethane (6 mL) at room temperature. The mixture was stirred at room temperature under protection of nitrogen for 2 h, then was concentrated to dryness by rotary evaporation at room temperature. The residue was quenched with saturated sodium bicarbonate solution, extracted twice with dichloromethane, dried over anhydrous sodium sulfate, concentrated to dryness by rotary evaporation, and the residue was purified by column chromatography (mobile phase: methanol/dichloromethane, 0-10%) to give 93 (147 mg, 77%). LC-MS (ESI): m/z 690.9(M+H)⁺; ¹H NMR (400M, DMSO-d₆): δ 10.27 (1H, s), 8.24-8.15 (1H, m), 8.02 (1H, t, J=12.0 Hz), 7.95 (1H, d, J=2.4 Hz), 7.62 (1H, t, J=9.2 Hz), 7.44 (1H, d, J=2.4 Hz), 5.88 (1H, t, J=6.4 Hz), 5.29 (1H, d, J=54.4 Hz), 4.20-3.91 (5H, m), 3.42(2H, t, J=6.0 Hz), 3.31-3.21 (5H, m), 3.20-2.97 (5H, m), 2.93-2.78 (1H, m), 2.24-1.55 (12H, m).

### Synthesis of Compound 94

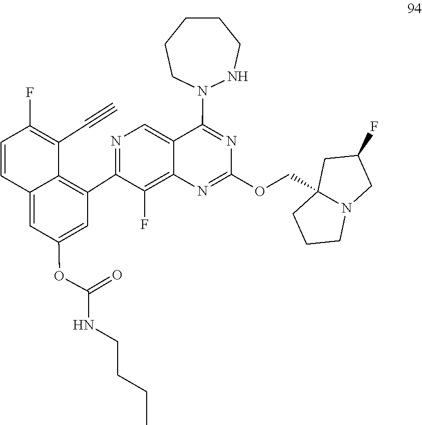 [SMILES: C#Cc1c(F)ccc2cc(OC(=O)NCCCC)cc(-c3ncc4c(N5CCCCCN5)nc(OC[C@@]56CCCN5C[C@H](F)C6)nc4c3F)c12]

Referring to the synthetic route of compound 69, n-butyl isocyanate was used instead of n-octyl isocyanate to obtain compound 94. LC-MS (ESI): m/z 688.9 (M+H)⁺; ¹H NMR (400M, DMSO-d₆) δ 10.26 (1H, s), 8.24-8.15 (1H, m), 8.00-7.89 (2H, m), 7.62 (1H, t, J=7.6 Hz), 7.50-7.40 (1H, m), 5.89 (1H, t, J=5.2 Hz), 5.29 (1H, d, J=42.8 Hz), 4.18-3.92 (5H, m), 3.24-2.97 (7H, m), 2.90-2.77 (1H, m), 2.22-1.58 (12H, m), 1.53-1.42 (2H, m), 1.39-1.27 (2H, m), 0.89 (3H, t, J=6.0 Hz).

### Synthetic Route of Compound 95

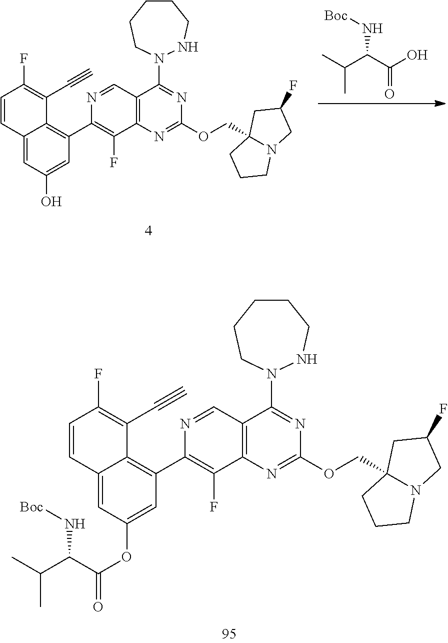

### Synthesis of Compound 95

To a solution of compound 4 (100 mg, 0.17 mmol) in dichloromethane (10 mL) was added Boc-L-valine (59 mg, 0.27 mmol) and N,N′-dicyclohexylcarbodiimide (77 mg, 0.37 mmol), followed by addition of 4-dimethylaminopyridine (33 mg, 0.27 mmol) at room temperature and the reaction mixture was stirred for 2 hours, then was added water and the aqueous phase was extracted with dichloromethane (30 mL). The organic phase was dried over anhydrous sodium sulfate, filtered, concentrated to dryness by rotary evaporation, and the crude was purified by flash column chromatography (mobile phase: dichloromethane: methanol=10:1/dichloromethane, 0-100%) to give 95 (125 mg, 93%). LC-MS (ESI): m/z 788.9 (M+H)⁺; ¹H NMR (400 MHz, CDCl₃): δ 10.28 (1H, s), 7.89 (1H, dd, J=9.2, 6.0 Hz), 7.77 (1H d, J=1.6 Hz,), 7.42 (1H, s), 7.37 (1H, t, J=8.8 Hz), 5.30 (1H, dd, J=53.2, 2.4H), 5.11 (1H d, J=8.8 Hz), 4.53 (1H, q, J=4.4 Hz), 4.30 (1H, dd, J=10.4, 6.4 Hz), 4.24-4.15 (3H, m), 4.11-4.02 (2H, m), 3.55-3.44 (1H, m), 3.36-3.15 (5H, m), 3.05-2.97 (1H,m), 2.89 (1H, d, J=1.2 Hz), 2.40-2.29 (2H, m), 2.26-2.16 (2H, m), 1.88-1.78 (7H, m), 1.49 (9H, s), 1.11 (3H, d, J=6.8 Hz), 1.05 (3H, d, J=6.9 Hz).

### Synthetic Route of Compound 96

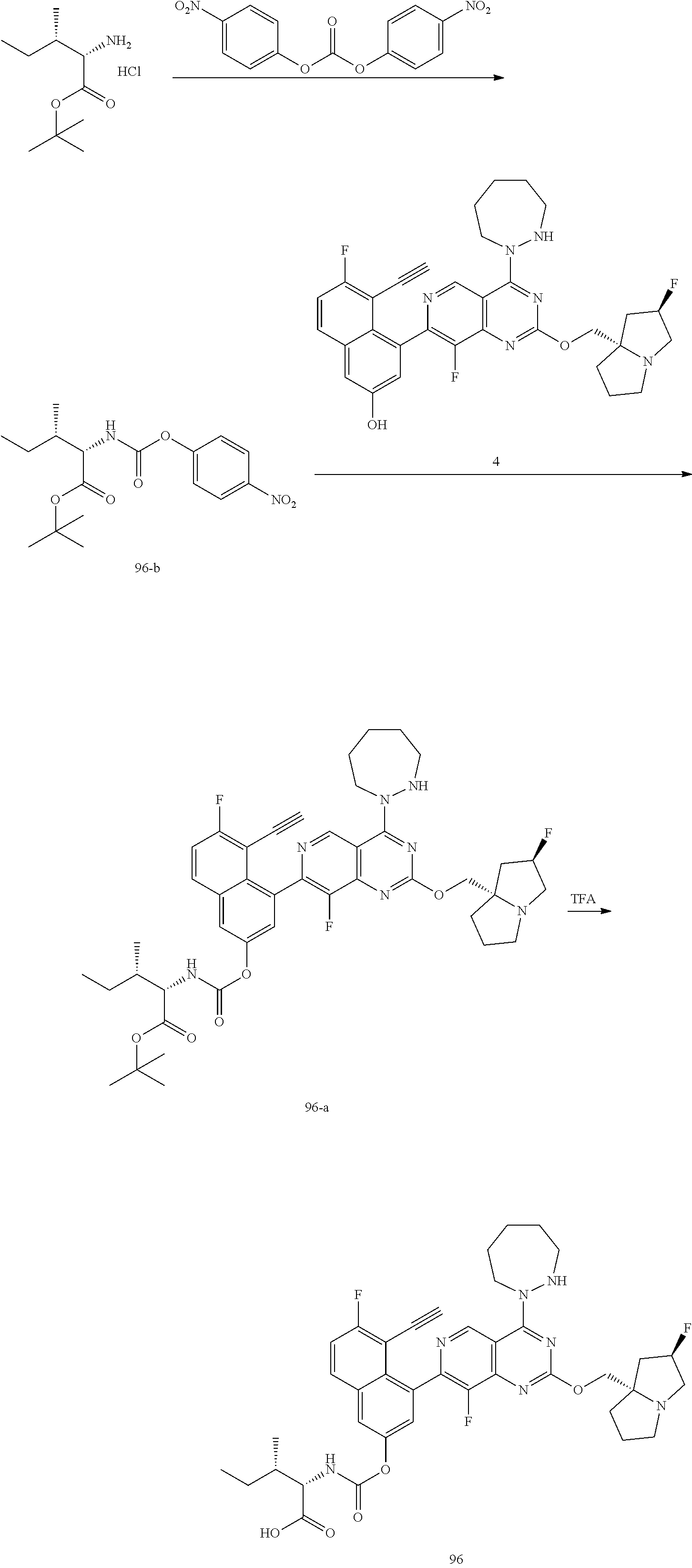

### Synthesis of Compound 96-b

To a solution of L-isoleucine tert-butyl ester hydrochloride (500 mg, 2.24 mmol) in acetonitrile (30 mL) was added DMAP (546 mg, 4.47 mmol) and bis(p-nitrophenyl) carbonate (748 mg, 2.46 mmol) at room temperature. The reaction mixture was heated at 50° C. for 3 hours. Water was added to the reaction mixture and the aqueous phase was extracted with dichloromethane (30 mL). The organic phase was dried over anhydrous sodium sulfate, filtered, concentrated to dryness by rotary evaporation, and the crude was purified by flash column chromatography (mobile phase: EA/PE, 0-50%) to give 96-b (618 mg, 78%).

### Synthesis of Compound 96-a

To a solution of compound 4 (224 mg, 0.38 mmol) in acetonitrile (15 mL) was added DMAP (186 mg, 1.52 mmol) and 96-b (536 mg, 1.52 mmol), and the reaction mixture was heated to 50° C. and stirred for 3 hours. The reaction mixture was concentrated to dryness by rotary evaporation, and the residue was added water and the aqueous phase was extracted with ethyl acetate (50 mL*2). The combined organic phases were dried over anhydrous sodium sulfate, filtered, concentrated to dryness by rotary evaporation, and the crude was purified by flash column chromatography (mobile phase: EA: PE, 0-50%) to give 96-a (179 mg, 59%). LC-MS (ESI): m/z 802.9 (M+H)⁺.

### Synthesis of Compound 96

TFA (3 mL) was added to a solution of 96-a (160 mg, 0.20 mmol) in DCM (6 mL) at room temperature and the reaction mixture was stirred for 4 hours at this temperature. Concentrated to dryness by rotary evaporation and the crude product was purified by reversed phase column (mobile phase: 0.5% TFA aqueous solution/acetonitrile, 0-40%) to give 96 (108 mg, 73%). LC-MS (ESI): m/z 746.8 (M+H)⁺; ¹H NMR (400 MHz, CDCl₃): δ 10.62 (1H, s), 7.94 (1H, dd, J=8.8, 5.6 Hz), 7.82 (1H, d, J=2 Hz), 7.38 (2H, t, J=8.8 Hz), 5.96 (1H, d, J=8.8 Hz), 5.93-5.80 (1H, m), 5.51 (1H, d, J=52.0 Hz), 4.71 (1H, d, J=9.6 Hz), 4.63 (1H, d, J=14 Hz), 4.38 (1H, d, J=10 Hz), 4.25 (1H, dd, J=8.8, 4 Hz), 4.10 (2H, dd, J=39.6, 14 Hz), 3.81-3.73 (1H, m), 3.40-3.24 (3H, m), 3.18-3.01 (2H, m), 2.90 (1H, s), 2.73 (1H, t, J=17.6 Hz), 2.64-2.56 (1H, m), 2.47-2.30 (1H, m), 2.21-1.97 (6H, m), 1.80-1.53 (5H, m), 1.32-1.23 (1H, m), 1.07 (3H, d, J=6.4 Hz), 0.99 (3H, t, J=7.2 Hz).

### Synthetic Route of Compound 97

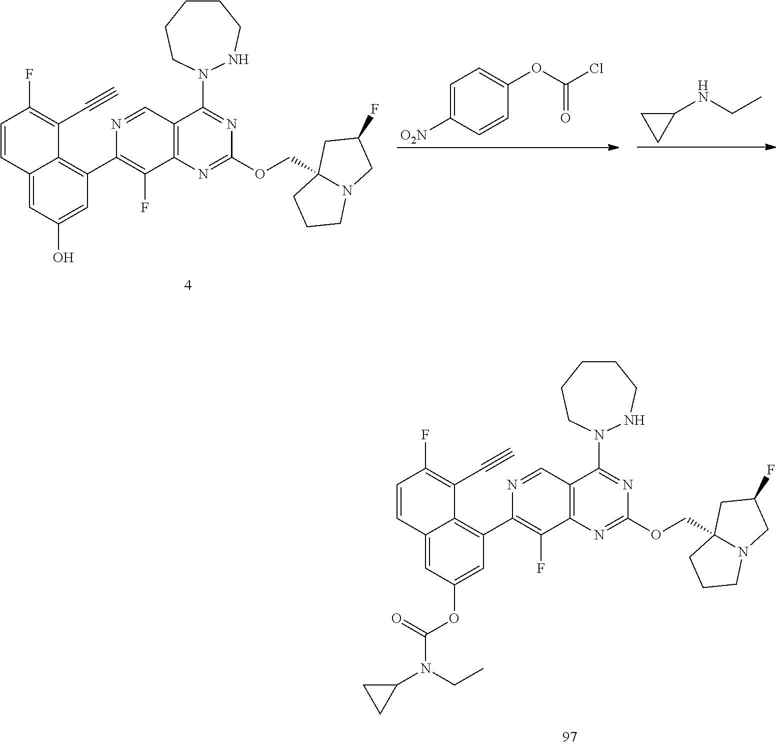

### Synthesis of Compound 97

To a solution of compound 4 (200 mg, 0.34 mmol) in DCM (6 mL) was added triethylamine (0.094 mL, 0.68 mmol) and p-nitrophenyl chloroformate (75 mg, 0.37 mmol) in an ice-salt bath. The reaction mixture was stirred at this temperature for 20 minutes, then was added N-cyclopropyl-N-ethylamine (43 mg, 0.51 mmol) and the reaction mixture was stirred at this temperature for 2 hours, then was added water and the aqueous phase was extracted with dichloromethane (30 mL). The organic phase was dried over anhydrous sodium sulfate, filtered, concentrated to dryness by rotary evaporation and the crude was purified by Prep-HPLC to give compound 97 (61 mg, 26%). LC-MS (ESI): m/z 700.3 (M+H)⁺; ¹H NMR (400 MHz, CDCl₃): δ 10.27 (1H, s), 7.89 (1H, dd, J=8.8, 5.6 Hz), 7.83 (1H, d, J=2 Hz), 7.48 (1H, d, J=2 Hz), 7.33 (1H, t, J=8.4 Hz), 5.30 (1H, d, J=54 Hz), 4.28 (1H, dd, J=10.4, 6.4 Hz), 4.23-4.14 (3H, m), 4.10-4.00 (1H, m), 3.52-3.41 (2H, m), 3.33-3.23 (2H, m), 3.23-3.13 (3H, m), 3.03-2.95 (1H, m), 2.88 (1H, s), 2.80-2.72 (1H, m), 2.30 (1H, s), 2.25-2.16 (2H, m), 2.00-1.76 (9H, m), 1.27 (3H, s), 0.88 (2H, d, J=6 Hz), 0.79 (2H, s).

### Synthesis of Compound 98

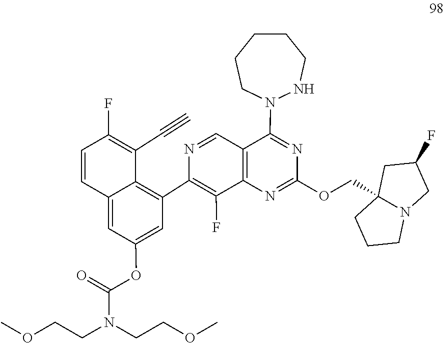 [SMILES: C#Cc1c(F)ccc2cc(OC(=O)N(CCOC)CCOC)cc(-c3ncc4c(N5CCCCCN5)nc(OC[C@@]56CCCN5C[C@H](F)C6)nc4c3F)c12]

Referring to the synthetic route of compound 97, compound 98 was obtained by using bis(2-methoxyethyl)amine instead of N-cyclopropyl-N-ethylamine. LC-MS (ESI): m/z 748.3 (M+H)⁺; ¹H NMR (400 MHz, CDCl₃): δ 10.27 (1H, s), 7.89 (1H, dd, J=7.2, 4.8 Hz), 7.81 (1H, d, J=2 Hz), 7.48 (1H, d, J=2 Hz), 7.34 (1H, t, J=7.2 Hz), 5.30 (1H, d, J=43.2 Hz), 4.29 (1H, dd, J=8, 5.2 Hz), 4.24-4.15 (3H, m), 4.11-4.03 (1H, m), 3.74-3.70 (2H, m), 3.66-3.59 (6H, m), 3.39 (6H, s), 3.33-3.24 (2H, m), 3.23-3.18 (3H, m), 3.02-2.96 (1H, m), 2.87 (1H, d, J=1.6 Hz), 2.30 (1H, s), 2.25-2.17 (2H, m), 2.03-1.76 (9H, m).

### Synthesis of Compound 99

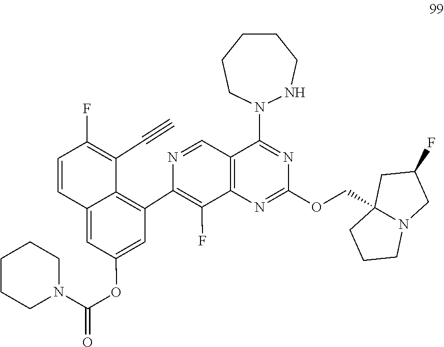 [SMILES: C#Cc1c(F)ccc2cc(OC(=O)N3CCCCC3)cc(-c3ncc4c(N5CCCCCN5)nc(OC[C@@]56CCCN5C[C@H](F)C6)nc4c3F)c12]

Referring to the synthetic route of compound 89, compound 99 was obtained using piperidine-1-carbonyl chloride instead of dimethylcarbamoyl chloride. LC-MS (ESI): m/z 700.5 (M+H)⁺.

### Synthetic Route of Compound 100

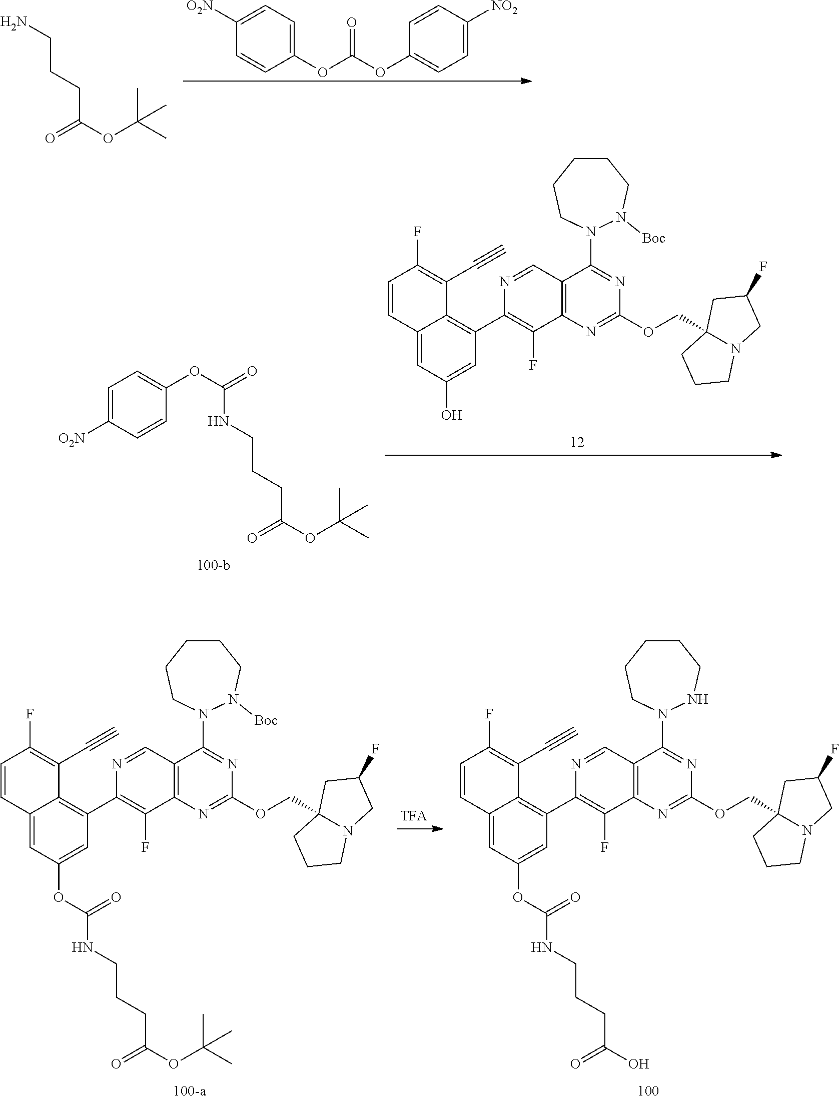

### Synthesis of Compound 100-b

A solution of tert-butyl 4-aminobutyrate (1000 mg, 6.28 mmol) and DMAP (1535 mg, 12.56 mmol) in acetonitrile (10 mL) was added dropwise to a solution of bis(p-nitrophenyl) carbonate (2102 mg, 6.91 mmol) in acetonitrile (10 mL) at room temperature. After addition, the mixture was stirred at room temperature for 1 hour. The reaction mixture was concentrated and the residue was purified by column chromatography (mobile phase: PE/EA 10/0 to 7/3) to give compound 100-b (700 mg, 34%). LC-MS (ESI): m/z 269.1 (M-56+H)⁺.

### Synthesis of Compound 100-a

Compound 12 (150 mg, 0.22 mmol), acetonitrile (15 mL) and DMAP (106 mg, 0.87 mmol) were added to a reaction vial at room temperature. After degassed and purged with N2, heated to 50° C., the mixture was added 100-b (318 mg, 0.98 mmol) in batches, and stirred at 50° C. overnight. The reaction mixture was concentrated and the residue was purified by column chromatography (mobile phase: DCM/MeOH 10/0 to 10/1) to give compound 100-a (110 mg, 58%). LC-MS (ESI): m/z 875.0 (M+H)⁺.

### Synthesis of Compound 100

100-a (110 mg, 0.13 mmol), DCM (4.5 mL) were added to a reaction vial at room temperature. The mixture was degassed and purged with nitrogen, then was added TFA (1.5 mL), and was stirred at room temperature for 3 hours. The reaction mixture was concentrated at room temperature and the residue was purified by prep-HPLC to give compound 100 (65 mg, 72%). LC-MS (ESI): m/z 718.4 (M+H)⁺.

### Synthesis of Compound 101

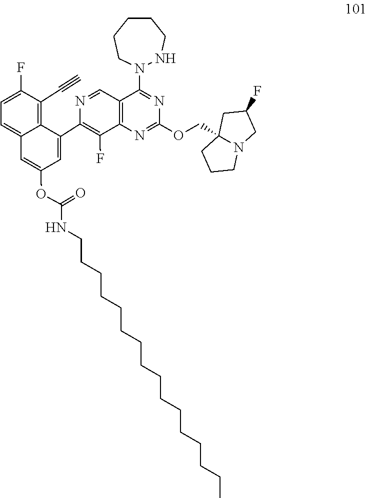 [SMILES: C#Cc1c(F)ccc2cc(OC(=O)NCCCCCCCCCCCCCCCC)cc(-c3ncc4c(N5CCCCCN5)nc(OC[C@@]56CCCN5C[C@H](F)C6)nc4c3F)c12]

Referring to synthetic route of compound 69, using hexadecyl isocyanate instead of n-octyl isocyanate to obtain compound 101. LC-MS (ESI): m/z=856.6[M+H]⁺; ¹H NMR (CDCl₃, 400 MHz): δ 10.25 (1H, s), 7.90-7.81 (1H, m), 7.78 (1H, s), 7.44 (1H, s), 7.35-7.27 (1H, m), 5.46-5.17 (2H, m), 4.41-3.87 (6H, m), 3.49-2.71 (11H, m), 2.40-2.12 (4H, m), 2.07-1.84 (6H, m), 1.83-1.63 (5H, m), 1.62-1.47 (3H, m), 1.44-1.27 (19H, m), 0.96-0.78 (3H, m).

### Synthetic Route of Compound 102

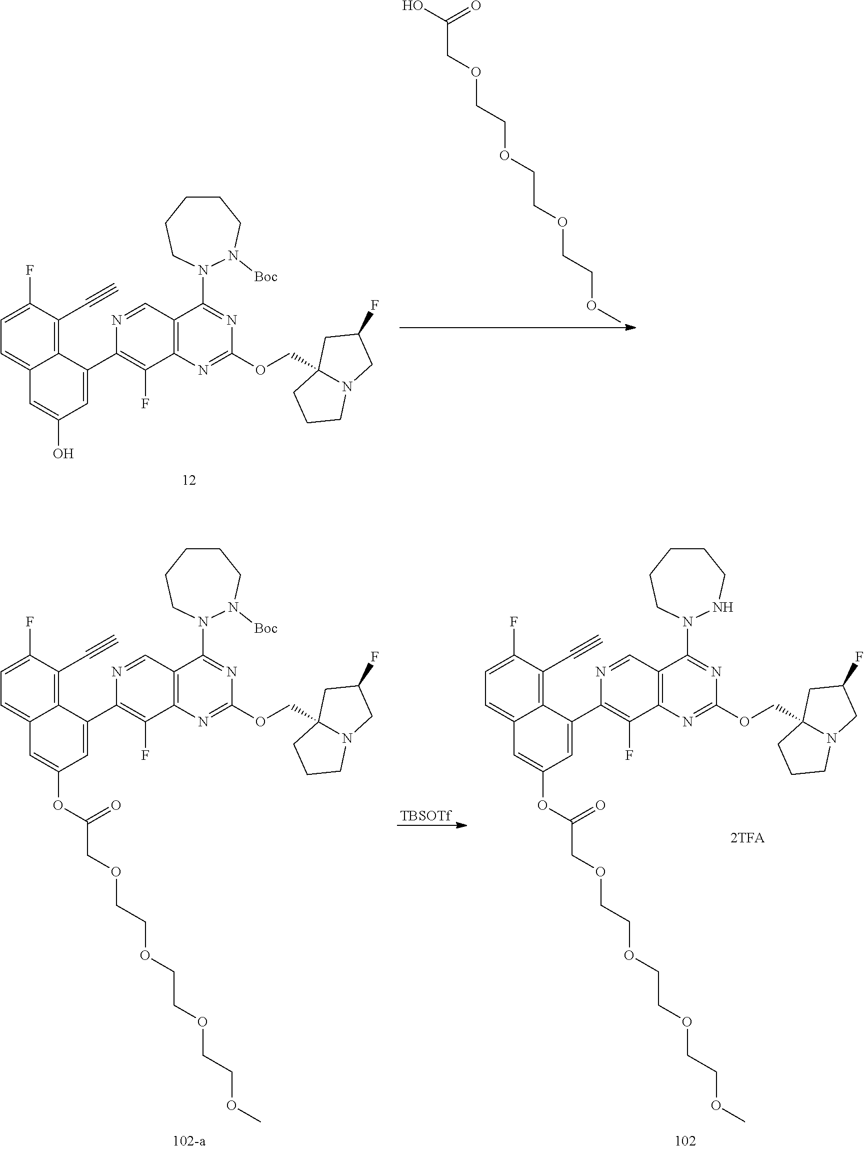

### Synthesis of Compound 102-a

Compound 3,6,9,12-tetraoxatridecanoic acid (36 mg, 0.16 mmol) was dissolved in 20 mL of DCM at room temperature. HATU (71 mg, 0.19 mmol), DIPEA (58 uL, 0.35 mmol) and DMAP (5.7 mg, 0.046) were added to the mixture in an ice-water bath, and the resulting mixture was stirred at 0° C. under protection of nitrogen for 10 min, then was added compound 12 (80 mg, 0.12 mmol) and the reaction mixture was warmed to room temperature and stirred overnight. The reaction mixture was added water (100 mL) at room temperature, extracted with DCM (100 mnL). The organic phase was washed with 10% NaHSO₄ solution (100 mL), saturated sodium bicarbonate solution (100 mL), brine (100 mL), dried over anhydrous sodium sulfate, filtered, and the filtrate was concentrated at reduced pressure. Compound 102-a (100 mg, 96%) was obtained. LC-MS (ESI): m/z=893.2[M+H]⁺.

### Synthesis of Compound 102

Two batches of compound 102-a (136 mg, 0.15 mmol) were dissolved in 5 mL of DCM at room temperature, followed by addition of tert-butyldimethylsilyl trifluoromethanesulfonate (500 uL) in an ice-water bath. The reaction mixture was stirred at 0° C. under protection of nitrogen for 60 min, then was added 10% NaHSO₄ solution (10 mL), warmed to room temperature and stirred for 1 hour. The reaction mixture was added 100 mL of saturated sodium bicarbonate solution, extracted with DCM (100 mL). The organic phase was dried over anhydrous sodium sulfate, filtered, and the filtrate was concentrated at reduced pressure, and the residue was purified by Prep-HPLC (Acid method, TFA) to give compound 102 (34.8 mg, 22%). LC-MS (ESI): m/z=793.5[M+H]⁺.

### Synthetic Route of Compound 103

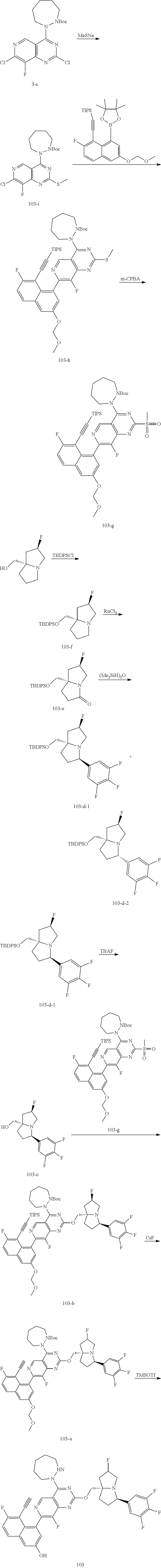

### Synthesis of compound 103-i

3-c (5.5 g, 13.21 mmol) and anhydrous potassium carbonate (1.83 g, 13.21 mmol) were added to a reaction flask. Placed in an ice-salt bath, the above mixture was added tetrahydrofuran (60 mL) and a solution of sodium methanethiol (1.6 g, 22.83 mmol) in water (16 mL) dropwise. After addition, the reaction mixture was stirred in an ice-salt bath to 5° C. for 5 hours, then was added ethyl acetate/petroleum ether (1:1), partitioned, and the aqueous phase was extracted once with ethyl acetate/petroleum ether (1:1). The organic phases were combined, washed with water, brine, dried over anhydrous sodium sulfate, filtered and evaporated to dryness. The solid was dissolved with a small amount of dichloromethane, removed most of the dichloromethane, slowly added n-heptane, sonicated for 10 s, left to stand still, partitioned off the supernatant, and the solid was washed twice more with n-heptane, concentrated to dryness by rotary evaporation to obtain 103-i (5.3 g, 94% yield).

LC-MS (ESI): m/z 428.3 (M+H)⁺.

### Synthesis of Compound 103-h

A reaction vial was charged with 103-i (5.0 g, 11.68 mmol), ((2-fluoro-6-(methoxymethoxy)-8-(4,4,5,5-tetramethyl-1,3,2-dioxaboraborolan-2-yl)naphthalen-1-yl)ethynyl) triisopropylsilane (8.38 g, 16.36 mmol), SPhos Pd G3 (0.50 g, 0.64 mmol), 1-propanol (150 mL) and a solution of anhydrous potassium phosphate (9.7 g, 45.69 mmol) in water (30 mL). The reaction mixture was degassed and purged with nitrogen gas 4 times and then heated and stirred at 75° C. for 16 hours. The reaction mixture was cooled to room temperature. Most of the solvent was removed by rotary evaporation. Ice - water was added, the aqueous phase was extracted twice with ethyl acetate/petroleum ether (1:1), the organic phases were combined, washed with water, brine, dried over anhydrous sodium sulfate, filtered, evaporated, and the residue was purified by column chromatography (mobile phase: petroleum ether/ethyl acetate, 100/0 to 80/20) to give compound 103-h (7.2 g, 79% yield). LC-MS (ESI): m/z 778.5 (M+H)⁺.

### Synthesis of Compound 103-g

The reaction flask was charged with 103-h (7.2 g, 9.25 mmol) and ethyl acetate (150 mL). m-CPBA (4.79 g, 27.76 mmol) was added to a reaction flask in an ice-water bath. The reaction mixture was stirred in ice-water bath for 3 hours and then m-CPBA (0.48 g, 2.78 mmol) was added. The reaction was quenched by adding saturated aqueous sodium sulfite and the aqueous phase was extracted with ethyl acetate three times. The organic phases were combined, washed with brine, dried over anhydrous sodium sulfate, filtered, evaporated and the residue was purified by column chromatography (mobile phase: petroleum ether/ethyl acetate, 100/0 to 0/100) to give compound 103-g (5.6 g, 75% yield). LC-MS(ESI): m/z 810.4 (M+H)⁺.

### Synthesis of Compound 103-f

((2R,7aS)-2-fluorohexahydro-1H-pyrrolizin-7a-yl)methanol (1.59 g, 10.00 mmol) and dichloromethane (20 mL) were added to a reaction flask. Placed in an ice-water bath, the reaction mixture was added imidazole (1.02 g, 14.98 mmol) and TBDPSCl (3.15 g, 11.46 mmol) slowly and dropwise. The reaction mixture was stirred at room temperature for 2 hours. The reaction was quenched by the addition of saturated aqueous sodium bicarbonate, partitioned, and the aqueous phase was extracted once with dichloromethane, the organic phases were combined, dried over anhydrous sodium sulfate, filtered, evaporated, and the residue was purified by column chromatography (mobile phases: petroleum ether, 100, and dichloromethane/methanol, 100/0 to 95/5) to give 103-f (3.8 g, 95%). LC-MS (ESI): m/z 398.3 (M+H)⁺.

### Synthesis of Compound 103-e

103-f (3.8 g, 9.56 mmol) and carbon tetrachloride (40 mL) were added to a reaction flask. A solution of ruthenium trichloride hydrate (0.38 g, 1.83 mmol) in water (40 mL) was added dropwise to the reaction mixture in an ice-water bath, followed by addition of sodium periodate (10.2 g, 47.69 mmol). The reaction mixture was heated and stirred at 25° C. for 2 hours. The reaction was quenched by the addition of water (40 mL), extracted twice with dichloromethane, the organic phases were combined, was added 5% aqueous sodium bisulfite, filtered through celite, the liquid was partitioned, the aqueous phase was extracted once with dichloromethane, the organic phases were combined, dried over anhydrous sodium sulfate, filtered, evaporated, and the residue was purified by column chromatography (mobile phase: petroleum ether/ethyl acetate, 100/0 to 67/33) to give the compound 103-e (3.0 g. Yield 76%). LC-MS (ESI): m/z 412.2 (M+H)⁺.

### Synthesis of Compounds 103-d-1 and 103-d-2

To a reaction vial A were added 3,4,5-trifluoroiodobenzene (1.20 g, 4.65 mmol) and anhydrous tetrahydrofuran (10 mL). Isopropylmagnesium chloride-lithium chloride 1.3 M THF (3.73 mL, 4.85 mmol) was added slowly and dropwise in an ice-water bath. The reaction mixture was stirred at room temperature for 30 min and allowed to stand to obtain Solution A. To a reaction vial B was added 103-e (950 mg, 2.31 mmol), carbonylbis(triphenylphosphine)iridium(I) chloride (47 mg, 0.060 mmol) and anhydrous toluene (12 mL). The mixture in the reaction vial B was sonicated until the ingredients were dissolved, then was added 1,1,3,3-tetramethyldisiloxane (465 mg, 3.46 mmol) dropwise in an ice-water bath. The reaction mixture was stirred in an ice-water bath for 35 minutes and then cooled to −78° C., was slowly added the solution A dropwise. Removed the cryogenic bath and the reaction mixture was slowly warmed for 45 min and then stirred in a water bath for 45 min. The reaction was quenched by adding a saturated ammonium chloride solution in an ice-water bath, water and a small amount of anhydrous sodium sulfate solid, and the reaction mixture was extracted once with ethyl acetate/petroleum ether (4:1). The organic phase was dried over anhydrous sodium sulfate, filtered, evaporated and the residue was purified by column chromatography (mobile phase: petroleum ether/ethyl acetate, 100/0 to 90/10) to give the crude product, which was again purified by column chromatography (mobile phase: petroleum ether/(10% ethyl acetate/petroleum ether containing 0.15% triethylamine), 100/0 to 75/25) to give compound 103-d-1 (450 mg, 37% yield).

LC-MS (ESI): m/z 528.2 (M+H)⁺. The collected mixture was purified by prep-HPLC to give compound 103-d-2 (380 mg, 31% yield). LC-MS(ESI): m/z 528.2 (M+H)⁺.

### Synthesis of Compound 103-c

103-d-1 (450 mg, 0.85 mmol) and tetrahydrofuran (4 mL) were added to a reaction flask, followed by dropwise addition of tetrabutylammonium fluoride (TBAF) trihydrate (1.2 mL, 1.20 mmol) tetrahydrofuran solution under protection of nitrogen. The reaction mixture was heated and stirred at 50° C. for 2 hours. The solvent was removed by rotary evaporation, and the residue was added saturated aqueous sodium bicarbonate solution. The aqueous phase was extracted three times with methanol/dichloromethane (10%) solution. The organic phases were combined, dried over anhydrous sodium sulfate, filtered, evaporated, and the crude was purified by column chromatography (mobile phase: dichloromethane/(dichloromethane/methanol/ammonia 10:1:0.2), 100/0 to 55/45) to afford compound 103-c (240 mg, 97% yield). LC-MS (ESI): m/z 290.2 (M+H)⁺.

### Synthesis of Compound 103-b

103-g (575 mg, 0.71 mmol) and 103-c (190 mg, 0.66 mmol) were added in a reaction flask. After degassed, the above mixture was added anhydrous toluene (15 mL). After degassed and purged with nitrogen gas for 3 times, the reaction mixture was added sodium tert-butoxide (192 mg, 2.00 mmol) in an ice-water bath. The reaction mixture was stirred in an ice-water bath for 1.5 h. The reaction mixture was purified by direct column chromatography (reaction mixture added to silica gel with slight stirring, mobile phase: petroleum ether/ethyl acetate, 100/0 to 65/35) to give 103-b (0.55 g, 82% yield). LC-MS (ESI): m/z 1019.7 (M+H)⁺.

### Synthesis of Compound 103-a

103-b (550 mg, 0.54 mmol) and anhydrous DMF (6 mL) were added to a reaction flask. After degassed and purged with nitrogen for 3 times, the reaction mixture was added cesium fluoride (1.20 g, 7.90 mmol), then was heated and stirred at 50° C. for 1 hour. The reaction mixture was add ice-water. The aqueous phase was extracted twice with ethyl acetate. The organic phase was washed twice with water, brine, dried over anhydrous sodium sulfate, filtered, and evaporated to obtain 103-a (0.46 g, 99% yield). LC-MS (ESI): m/z 863.7 (M+H)⁺.

### Synthesis of Compound 103

103-a (460 mg, 0.53 mmol) and anhydrous dichloromethane (10 mL) were added to a reaction flask, followed by addition of TMSOTf (0.50 mL, 2.75 mmol) in an ice-water bath. The reaction mixture was stirred in the ice-water bath for 4 hours, then was placed in refrigerator to freeze overnight. After stirred in an ice-water bath for 3 hours, the reaction mixture was added 10% aqueous sodium bisulfate solution (10 mL), dichloromethane (50 mL) and water (10 mL), and the reaction mixture was stirred for 20 minutes in the ice-water bath and then for 1 hour at room temperature. Separate the liquid, the aqueous phase was extracted with dichloromethane once, the organic phases were combined, dried over anhydrous sodium sulfate, filtered, evaporated, purified by column chromatography (mobile phase: dichloromethane/methanol, 100/0 to 90/10), the product fractions were collected and concentrated to dryness, and lyophilized to obtain compound 103 (190 mg, 50% yield). LC-MS(ESI): m/z 719.3(M+H)⁺.

### Synthetic Route of Compound 104

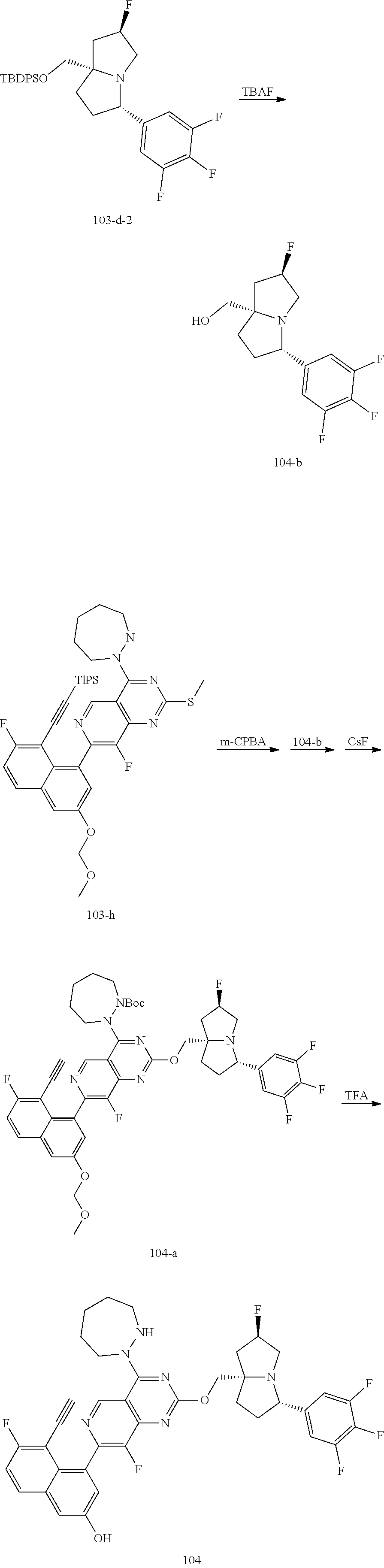

### Synthesis of Compound 104-b

103-d-2 (380 mg, 0.72 mmol) and tetrahydrofuran (4 mL) were added to a reaction flask, followed by dropwise addition of a solution of tetrabutylammonium fluoride (TBAF) trihydrate (1.0 mL, 1.00 mmol) in tetrahydrofuran under protection of nitrogen. The reaction mixture was heated and stirred at 50° C. for 2 hours. The solvent was removed by rotary evaporation, the residue was added saturated aqueous sodium bicarbonate solution, extracted with methanol/dichloromethane (10%) solution 3 times, the organic phases were combined, dried over anhydrous sodium sulfate, filtered, evaporated, and the crude was purified by column chromatography (mobile phase: dichloromethane/(dichloromethane/methanol/ammonia 10:1:0.2), 100/0 to 65/35) to give compound 104-b (85 mg, 40% yield). LC-MS (ESI): m/z 290.1 (M+H)⁺.

### Synthesis of Compound 104-a

103-h (100 mg, 0.13 mmol) and dichloromethane (4 mL) were added to a reaction flask and m-CPBA (45 mg, 0.26 mmol) was added in an ice-water bath. The reaction mixture was stirred for 2 h in an ice-water bath and then the reaction was quenched by the addition of saturated aqueous sodium bisulfite, extracted twice with ethyl acetate, the organic phases were combined, washed with brine, dried over anhydrous sodium sulfate, filtered, evaporated to dryness, and the residue was purified by column chromatography (mobile phase: petroleum ether/ethyl acetate, 100/0 to 0/100) to give a brown solid. This solid, 104-b (42 mg,0.15 mmol), DABCO (3 mg, 0.027 mmol) and cesium carbonate (84 mg, 0.26 mmol) were combined with anhydrous acetonitrile (4 mL) in a reaction vial. After degassed and purged with nitrogen for three times, the reaction mixture was stirred at 50° C. overnight. Cesium fluoride (118 mg, 0.78 mmol) and tetrabutylammonium bromide (TBAB) (3 mg, 0.009 mmol) were added. After degassed and purged with nitrogen for three times, the reaction mixture was stirred at 50° C. for 2 hours. The solvent was removed by rotary evaporation, and the residue was added ethyl acetate, washed with water, brine, dried over anhydrous sodium sulfate, filtered, and evaporated to give the crude product of 104-a (0.11 g, 84% yield). LC-MS (ESI): m/z 863.7 (M+H)⁺.

### Synthesis of Compound 104

To a reaction flask were added 104-a (110 mg, 0.13 mmol), dichloromethane (3 mL) and anisole (0.3 mL). Trifluoroacetic acid (0.3 mL) was added dropwise in an ice-water bath. The reaction mixture was stirred at room temperature for 1.5 hours. The solvent was removed by rotary evaporation, and the residue was added methyl tert-butyl ether. The supernatant was partitioned off, the residue was washed once with methyl tert-butyl ether and concentrated to dryness by rotary evaporation, and the crude was purified by Prep-HPLC to give 104 (11.2 mg, 12% yield). LC-MS (ESI): m/z 719.4(M+H)⁺.

### Synthetic Route of Compound 105

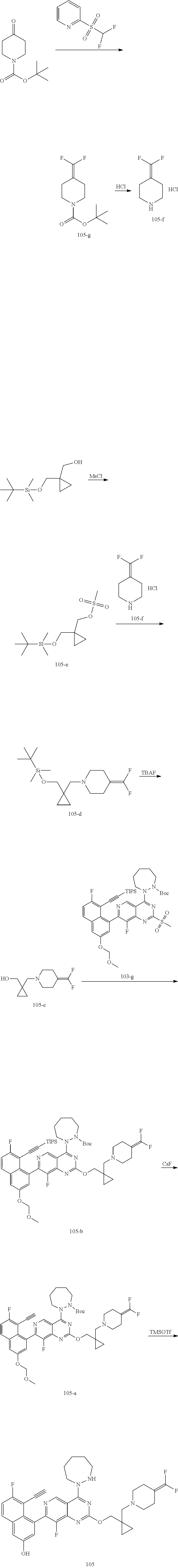

### Synthesis of Compound 105-g

Potassium tert-butanolate (1.69 g, 15.06 mmol) was added to 15 mnL of DMF at room temperature and cooled to −40° C. (dry ice acetonitrile). A solution of compound N-Boc-4-piperidone (2.0 g, 10.04 mmol) and compound 2-((difluoromethyl)sulfonyl)pyridine (1.65 g, 8.54 mmol) in DMF (10 mL) was added dropwise to the above mixture at −40° C. After addition, the reaction mixture was stirred at −40° C. under protection of nitrogen for 30 minutes. The reaction was quenched with saturated sodium bicarbonate solution (5 mnL) and 3M hydrochloric acid (13 mL) under protection of nitrogen at -40° C. The reaction mixture was warmed to room temperature naturally and stirred for 30 minutes. To the mixture was added 100 mL of water with ethyl acetate (100 mL*2), the organic phase was washed with brine (200 mL*5), dried over anhydrous sodium sulfate, filtered, and the filtrate was concentrated at reduced pressure, and the residue was separated and the residue was purified by a flash column chromatography (EA/PE=0:50%). Compound 105-g (1.6 g, 80%) was obtained. LC-MS (ESI): m/z=178.1 [M-56+H]⁺.

### Synthesis of Compound 105-f

Compound 105-g (1.6 g, 6.86 mmol) was dissolved in hydrogen chloride/1,4-dioxane solution (10 mL) at room temperature and stirred at room temperature for 1 hour. The reaction mixture was concentrated at reduced pressure and dried under vacuum to give compound 105-f (1.16 g, 100%).

LC-MS (ESI): m/z=134.2[M+H]⁺.

### Synthesis of Compound 105-e

Compound (1-(((tert-butyldimethylsilyl)oxy)methyl)cyclopropyl)methanol (450 mg, 2.08 mmol) was dissolved in dichloromethane (10 mL) in an ice-water bath at room temperature and DIPEA (1.03 mL, 6.24 mmol) and methanesulfonyl chloride (241 uL, 3.12 mmol) were added and the reaction mixture was naturally warmed to room temperature and stirred for 16 hours. The reaction mixture was quenched with saturated ammonium chloride solution (50 mL). The aqueous phase was extracted with dichloromethane (100 mL), the organic phase was washed sequentially with 10% NaHSO₄ solution (50 mL), saturated sodium bicarbonate solution (100 mL), brine (100 mL), dried over anhydrous sodium sulfate, filtered, and the filtrate was concentrated at reduced pressure and dried at vacuo to give compound 105-e (crude product. 600 mg, 98%). The crude product was directly used in the next step.

### Synthesis of Compound 105-d

Compound 105-e (600 mg, 2.04 mmol) was dissolved in acetonitrile (25 mL) at room temperature, compound 105-f (380 mg, 2.24 mmol), potassium carbonate (845 mg, 6.11 mmol) and sodium iodide (916 mg, 6.11 mmol) were added sequentially, and the reaction mixture was stirred for 2 hours at 80° C. under protection of nitrogen. The reaction mixture was cooled to room temperature, filtered and the filtrate was concentrated at reduced pressure. The residue was dissolved in 100 mL of ethyl acetate. The mixture was washed sequentially with water (100 mL), 10% NaHSO₄ solution (50 mL), saturated sodium bicarbonate solution (100 mL), brine (100 mL), dried over anhydrous sodium sulfate, filtered, the filtrate was concentrated at reduced pressure, and the residue was purified by a flash column chromatography (EA/PE=0-50%) to give Compound 105-d (398 mg, 59%). LC-MS (ESI): m/z=332.3[M+H]⁺.

### Synthesis of Compound 105-c

Compound 105-d (388 mg, 1.17 mmol) was dissolved in THF (20 mL) at room temperature, a solution of TBAF in THF (1M, 1.76 mL, 1.76 mmol) was added, and the reaction mixture was stirred under protection of nitrogen for 18 h at room temperature. The reaction mixture was concentrated at reduced pressure and the residue was purified by a flash column chromatography (MeOH/DCM=0-5%) to afford compound 105-c (218 mg, 86%). LC-MS (ESI): m/z=218.2[M+H]⁺.

### Synthesis of Compound 105-b

Compound 103-g (100 mg, 0.12 mmol) was dissolved in anhydrous toluene (10 mL) at room temperature, compound 105-c (32 mg, 0.15 mmol) and sodium tert-butanol (59 mg, 0.62 mmol) were added sequentially in an ice-water bath and the reaction mixture was stirred at 0° C. under protection of nitrogen for 2 hours. To the reaction mixture, saturated ammonium chloride solution (100 mL) was added to quench the reaction. The aqueous phase was extracted with ethyl acetate (100 mL). The organic phase was washed with brine (100 mL), dried over anhydrous sodium sulfate, filtered, and the filtrate was concentrated at reduced pressure, and the residue was separated and the residue was purified by a flash column chromatography (EA/PE=0-50%) to give compound 105-b (100 mg, 88%). 1c -MS (ESI): m/z=947.5[M+H]⁺.

### Synthesis of Compound 105-a

Compound 105-b (100 mg, 0.11 mmol) was dissolved in DMF (5 mL) at room temperature, cesium fluoride (321 mg, 2.11 mmol) was added, and the reaction mixture was stirred at room temperature under protection of nitrogen for 2 h. The reaction was quenched by adding water (100 mL), and the aqueous phase was extracted with ethyl acetate (100 mL). The organic phase was washed with brine (100 mL*5), dried over anhydrous sodium sulfate, filtered, and the filtrate was concentrated at reduced pressure and dried at vacuo to give compound 105-a (crude, 90 mg), and the crude was used directly in the next step. LC-MS (ESI): m/z=791.4 [M+H]+.

### Synthesis of Compound 105

Compound 105-a (90 mg, 0.11 mmol) was dissolved in DCM (5 mL) at room temperature, trimethylsilyl trifluoromethanesulfonate (413 uL, 2.28 mmol) was added in an ice-water bath, and the reaction mixture was stirred under protection of nitrogen for 1 hr in an ice-water bath, then was added 10% NaHSO₄ solution (9 mL). The resulting mixture was stirred at room temperature for 30 min, then was added saturated sodium bicarbonate solution (100 mL), extracted with DCM (100 mL), dried over anhydrous sodium sulfate, filtered, and the filtrate was concentrated at reduced pressure, and the residue was isolated and purified (alkaline method, ammonium bicarbonate) by Prep-HPLC to give compound 105 (33 mg, 46%). LC-MS (ESI): m/z=647.2[M+H]⁺.

### Synthetic Route of Compound 106

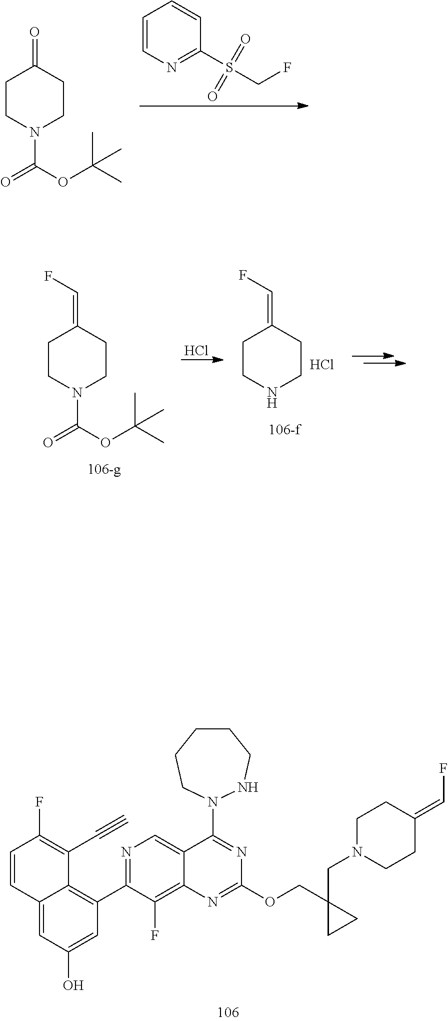

### Synthesis of Compound 106-g

Compound 2-((fluoromethyl)sulfonyl)pyridine (4.4 g, 25.12 mmol) was dissolved in 50 mL of anhydrous THF at room temperature under protection of nitrogen. Cooled in a dry ice-acetone bath, the reaction mixture was added potassium bis(trimethylsilyl)amide (1M, 30.11 mL, 30.11 mmol), stirred for 30 min in a dry ice-acetone bath and then was added N-Boc- 4-piperidone (5.0 g, 25.09 mmol). The reaction mixture was stirred in a dry ice-acetone bath for 3 hours and then stirred at room temperature for 1 hour. The reaction mixture was cooled in an ice-water bath and quenched with saturated ammonium chloride solution (30 mL), then was added 3M hydrochloric acid (50 mL) and stirred at room temperature for 1 hour. The reaction mixture was extracted with ethyl acetate (50 mL*2), the organic phase was washed with brine (200 mL), dried over anhydrous sodium sulfate, filtered, the filtrate was concentrated at reduced pressure and the residue was separated and the residue was purified by a flash column chromatography (EA/PE=0:50%) to obtain compound 106-g (2.6 g, 48%). LC-MS (ESI): m/z=160.2 [M-56+H]⁺.

### Synthesis of Compound 106-f

Compound 106-g (2.6 g, 12.08 mmol) was dissolved in hydrogen chloride /1,4-dioxane (20 mL) at room temperature and stirred for 1 hour at room temperature. The reaction mixture was concentrated at reduced pressure and dried under vacuum to give compound 106-f (1.8 g, 98%). LC-MS (ESI): m/z=116.2[M+H]⁺.

### Synthesis of Compound 106

Referring to the synthetic route of compound 105, 106-f was used instead of 105-f to obtain compound 106. LC-MS (ESI): m/z=629.3[M+H]⁺; ¹H NMR (CDCl₃, 400 MHz): δ 10.20 (1H, s), 7.57-7.47 (1H, m), 7.19 (1H, s), 7.11 (1H, t, J=9.2 Hz), 7.00 (1H, s), 6.37 (1H, d, J=86.8 Hz), 4.45 (1H, d, J=10.0 Hz), 4.29 (2H, d, J=10.0 Hz), 4.06 (2H, s), 3.14 (2H, s), 2.78 (1H, s), 2.10-2.18 (7H, m), 2.14-1.88 (4H, m), 1.85-1.66 (6H, m), 0.76-0.63 (2H, m), 0.55-0.36 (2H, m).

### Synthetic Route of Compound 107

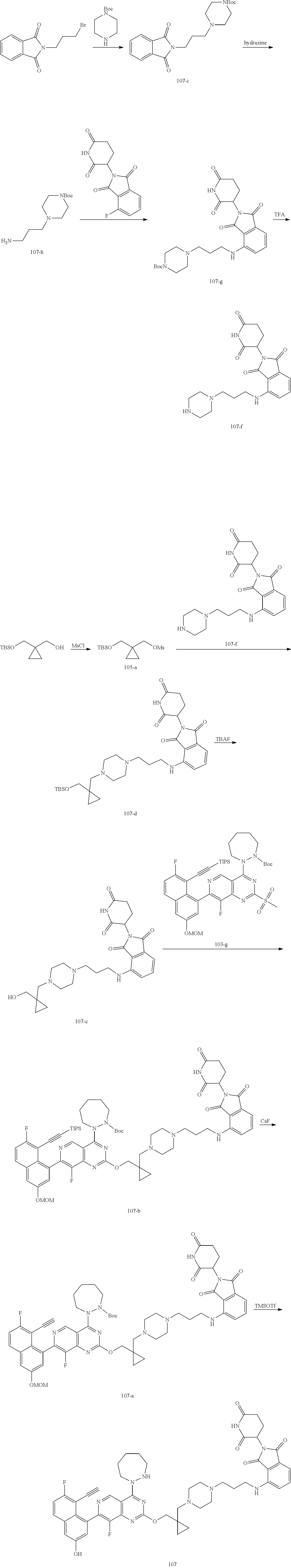

### Synthesis of Compound 107-i

N-(3-bromopropyl)phthalimide (3.00 g, 11.19 mmol), N-Boc-piperazine (2.50 g, 13.43 mmol), N,N-diisopropylethylamine (2.89 g, 22.38 mmol), and acetonitrile (15 mL) were added to a reaction flask, and the reaction mixture was stirred for 3 hours by heating at 70° C. The reaction mixture was cooled to room temperature, was added water (30 mL) and extracted with ethyl acetate (60 mL). The organic phases were combined, dried over anhydrous sodium sulfate, filtered, and the filtrate was concentrated to dryness by rotary evaporation to give 107-i (3.7 g, 89%). LCMS (ESI): m/z 374.5 (M+H)⁺.

### Synthesis of Compound 107-h

107-i (3.50 g, 9.37 mmol), ethanol (35 mml), hydrazine hydrate (85%, 4.69 g) were added in a reaction flask and heated at 60° C. with stirring for 1 h. The reaction mixture was cooled down to room temperature, filtered, and the filtrate was concentrated to dryness by rotary evaporation to give 107-h (2.10 g, 92%). LCMS (ESI): m/z 244.5 (M+H)⁺.

### Synthesis of Compound 107-g

107-h (2.00 g, 8.22 mmol), 2-(2,6-dioxo-piperidin-3-yl)-4-fluoroisoindole-1,3-dione (2.27 g, 8.22 mmol) and N-methylpyrrolidinone (20 mL) were added to a reaction vial and the reaction mixture was heated to 85° C. and stirred for 3 hours. The reaction mixture was cooled to room temperature, was added water (40 mL) and extracted with ethyl acetate (60 mL). The organic phases were combined, dried over anhydrous sodium sulfate, filtered, and the filtrate was concentrated to dryness by rotary evaporation and purified by column chromatography (mobile phase: ethyl acetate: petroleum ether=0˜30%) to give 107-g (1.90 g, 46%). LCMS(ESI): m/z 500.7 (M+H)⁺.

### Synthesis of Compound 107-f

107-g (1.90 g, 3.80 mmol) and dichloromethane (20 mL) were added to a reaction flask and cooled down to 0-5° C. in an ice-water bath. Trifluoroacetic acid (4.34 g, 38.03 mmol) was slowly added dropwise to it and kept stirring at 0˜5° C. for 2 hours. The reaction mixture was concentrated directly to dryness, and the residue was solubilized with dichloromethane (40 mL), and the pH was adjusted to 8˜9 with saturated aqueous sodium carbonate solution. The organic phase was separated and concentrated to give 107-f (1.34 g, 88%). LCMS(ESI): m/z 400.2 (M+H)⁺.

### Synthesis of Compound 105-e

To a reaction vial at room temperature was added 1-(tert-butyldimethylsilyloxymethyl)cyclopropyl]methanol (1000 mg, 4.62 mmol) and DCM (20 mL). After degassed and purged with nitrogen, the mixture was added DIPEA (1792 mg, 13.86 mmol) and methanesulfonyl chloride (794 mg, 6.93 mmol) in an ice-water bath. After addition, the reaction was warmed to room temperature naturally overnight. The reaction mixture was quenched with saturated aqueous ammonium chloride and the aqueous phase was extracted with DCM. The organic phases were combined, washed with saturated aqueous sodium bicarbonate, washed with water, brine, dried over anhydrous sodium sulfate and concentrated to give the crude compound 105-e (1400 mg).

### Synthesis of Compound 107-d

To a reaction flask at room temperature were added crude 105-e (300 mg, 0.99 mmol), acetonitrile (5 mL), 107-f (250 mg, 0.63 mmol), sodium iodide (281 mg, 1.88 mmol) and potassium carbonate (259 mg, 1.88 mmol). After degassed and purged with nitrogen, the mixture was heated at 80° C. for 3 hours. The reaction mixture was concentrated, purified by column chromatography (mobile phase: PE/EA 10/0 to 0/10) to give compound 107-d (70 mg, 19%). LC-MS (ESI): m/z 598.4 (M+H)⁺.

### Synthesis of Compound 107-c

107-d (70 mg, 0.12 mmol), THF (5 mL), tetrabutylammonium fluoride (74 mg, 0.23 mmol) were added to a reaction vial at room temperature. After degassed and purged with nitrogen, the mixture was heated at 50° C. for 8 h. The reaction mixture was concentrated to give the crude compound 107-c (110 mg). LC-MS (ESI): m/z 484.8 (M+H)⁺.

### Synthesis of Compound 107-b

To a reaction flask at room temperature was added 103-g (140 mg, 0.17 mmol), toluene (5 mL), and crude 107-c (110 mg, 0.12 mmol). After degassed and purged with nitrogen, cooled down to 0° C., the mixture was added sodium tert-butoxide (34 mg, 0.35 mmol), and stirred at 0° C. for 3 hours. The reaction mixture was mixed with silica gel and purified by column chromatography (mobile phase: DCM/MeOH(NH₃) 10/0 to 10/1) to give compound 107-b (10 mg, 7%). LC-MS (ESI): m/z 1213.6 (M+H)⁺.

### Synthesis of Compound 107-a

107-b (10 mg, 0.01 mmol), DMF (1 mL) and cesium fluoride (13 mg, 0.08 mmol) were added to a reaction flask at room temperature. After degassed and purged with nitrogen, The mixture was stirred at 50° C. for 1 h. The reaction mixture was blown to dryness with nitrogen, dissolved with THF and filtered. The filtrate was concentrated to give the crude compound 107-a (12 mg). LC-MS (ESI): m/z 1057.6 (M+H)⁺.

### Synthesis of Compound 107

To a reaction vial at room temperature was added crude 107-a (12 mg, 0.01 mmol) and DCM (2 mL). After degassed and purged with nitrogen and cooled down to 0° C., the mixture was added trimethylsilyl trifluoromethanesulfonate (0.2 mL) dropwise. It was stirred for 3 hours, then was added 10% aqueous sodium bisulfate (1 mL) and stirred for 1 hour. The reaction mixture was made basic with saturated aqueous sodium bicarbonate and the aqueous phase was extracted with DCM. The organic phases were combined, washed with water, brine, dried, concentrated and purified by prep-HPLC to give compound 107 (3.5 mg, 47%). LC-MS (ESI): m/z 913.5 (M+H)⁺.

### Synthetic Route of Compound 108

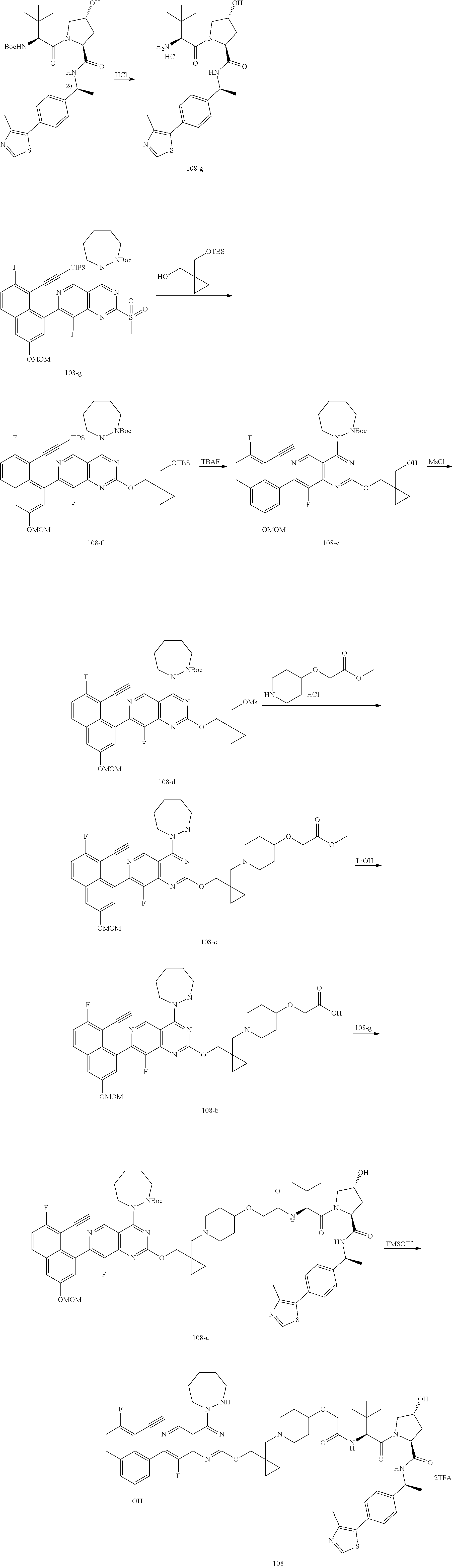

### Synthesis of Compounds 108-g

Compound ((S)-1-((2S,4R)-4-hydroxy-2-(((S)-1-(4-(4-methylthiazol-5-yl)phenyl)ethyl)carbamoyl)pyrrolidinocyclobutan-1-yl)-3,3-dimethyl-1-oxobutan-2-yl)carbamic acid tert-butyl ester (100 mg, 0.18 mmol) was dissolved in 5 mL of dichloromethane at room temperature, then was added hydrogen chloride /1,4-dioxane (10 mL, 4 M). The reaction mixture was stirred at room temperature overnight. The reaction mixture was concentrated at reduced pressure and the residue was dried under vacuum for 2 h to give compound 108-g (88 mg, 100%).

LC-MS (ESI): m/z=445.3[M+H]⁺.

### Synthesis of Compound 108-f

Compound 103-g (200 mg, 0.25 mmol) and compound (1-(((tert-butyldimethylsilyl)oxy)methyl)cyclopropyl)methanol (80 mg, 0.37 mmol) were dissolved in 10 mL of anhydrous toluene at room temperature. Placed in an ice-water bath, the above mixture was added sodium tert-butoxide (119 mg, 1.24 mmol) and stirred in an ice-water bath for 2 hours in an ice-water bath. The reaction mixture was added saturated ammonium chloride (50 mL), extracted with ethyl acetate (100 mL). The organic phase was dried over anhydrous sodium sulfate, filtered, and the filtrate was concentrated at reduced pressure, and the crude product was isolated and the residue was purified by a flash column chromatography (EA/PE=0-50%) to give compound 108-f (210 mg, 89%). LC-MS (ESI): m/z=946.5[M+H]⁺.

### Synthesis of Compound 108-e

Compound 108-f (210 mg, 0.22 mmol) was dissolved in 10 mL of THF at room temperature, tetrabutylammonium fluoride tetrahydrofuran solution (1 mL, 1.0 mmol, 1 M) was added, and the reaction mixture was stirred for 2 h at room temperature. The reaction mixture was concentrated at reduced pressure and the crude product was isolated and the residue was purified by flash column chromatography (MeOH/DCM=0-5%) to afford compound 108-e (140 mg, 94%). LC-MS (ESI): m/z=676.5[M+H]⁺.

### Synthesis of Compound 108-d

Compound 108-e (140 mg, 0.21 mmol) was dissolved in 10 mL of dichloromethane at room temperature, diisopropylethylamine (171 uL, 1.04 mmol) and methanesulfonyl chloride (32 uL, 0.41 mmol) were added to the mixture in an ice-water bath and the reaction mixture was stirred for 1.5 h at room temperature. The reaction mixture was concentrated at reduced pressure and the residue was purified by flash column chromatography (EA/PE=0-50%) to give compound 108-d (125 mg, 79%). LC-MS (ESI): m/z=754.4[M+H]⁺.

### Synthesis of Compound 108-c

Compound 108-d (110 mg, 0.15 mmol) was dissolved in 20 mL of anhydrous acetonitrile at room temperature, followed by addition of 2-(piperidin-4-yloxy)acetic acid methyl ester hydrochloride (46 mg, 0.22 mmol), potassium carbonate (101 mg, 0.73 mmol), and sodium iodide (109 mg, 0.73 mmol). The reaction mixture was stirred at 80° C. under protection of nitrogen for 2 hours. The reaction mixture was cooled to room temperature, filtered, the filtrate was concentrated at reduced pressure and the residue was purified by flash column chromatography (MeOH/DCM=0-10%) to give compound 108-c (100 mg, 80%). LC-MS (ESI): m/z=831.5[M+H]⁺.

### Synthesis of Compound 108-b

Compound 108-c (100 mg, 0.12 mmol) was dissolved in 5 mL of THF at room temperature, methanol (5 mL), water (2.5 mL), and lithium hydroxide monohydrate (25 mg, 0.60 mmol) were added, and the reaction mixture was stirred for 2 h at room temperature. The reaction mixture was concentrated at reduced pressure, and the residue was suspended in 20 mL of water. Cooled in an ice-water bath, it was adjusted pH to 3 with 1 M hydrochloric acid. The mixture was extracted with a mixture of solvents (DCM/MeOH=10:1, 50 mL*3), the organic phase was dried over anhydrous sodium sulfate, filtered, and the filtrate was concentrated at reduced pressure, and the residue was dried at vacuo overnight to afford compound 108-b (93 mg, 95%). LC-MS (ESI): m/z=817.5[M+H]⁺.

### Synthesis of Compound 108-a

Compound 108-b (90 mg, 0.11 mmol) was dissolved in 5 mL of DMF at room temperature, followed by addition of HATU (63 mg, 0.17 mmol), DIPEA (55 uL, 0.33 mmol) in an ice-water bath. The reaction mixture was stirred under protection of nitrogen in an ice-water bath for 10 min, then was added a solution of compound 108-g (80 mg, 0.17 mmol) in DMF (3 mL containing 110 μL of DIPEA), and the reaction mixture was stirred in an ice-water bath for 30 min. The reaction mixture was poured into 50 mL of water, filtered, and the filter cake was washed with 20 mL of water, and the solid was collected and then was purified by a flash column chromatography (MeOH/DCM=0-10%) to give compound 108-a (115 mg, 84%). LC-MS (ESI): m/z=1243.5 [M+H]⁺.

### Synthesis of Compound 108

Compound 108-a (60 mg, 0.048 mmol) was dissolved in 5 mL of DCM at room temperature and trimethylsilyl trifluoromethanesulfonate (500 uL, 2.76 mmol) was added to the above mixture in an ice-water bath and the reaction mixture was stirred under protection of nitrogen in the ice-water bath for 1 hr. To the reaction mixture was added 5 mL of methanol and 10% sodium bisulfate solution (5 mL), and the reaction mixture was stirred for 2 hours in an ice-water bath. The reaction mixture was added 20 mL of saturated sodium bicarbonate solution, extracted with dichloromethane (50 mL), the organic phase was dried over anhydrous sodium sulfate, filtered and the filtrate was concentrated at reduced pressure. The residue was purified by Prep-HPLC (NH₄HCO₃) and then by Prep-HPLC (TFA) to give compound 108 (24.8 mg, 39%), LC-MS (ESI): m/z=1099.5 [M+H]⁺.

### Synthetic Route of Compound 109

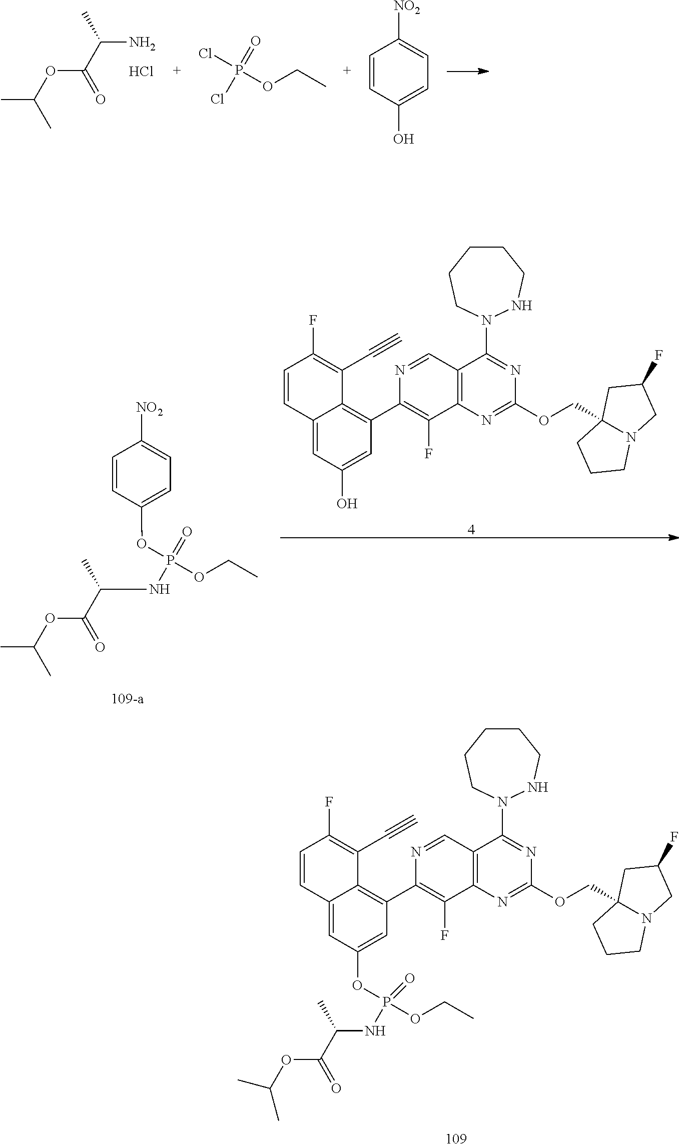

### Synthesis of Compound 109-a

To a solution of L-alanine isopropyl ester hydrochloride (2000 mg, 11.93 mmol) in DCM (40 mL) was added ethyl dichlorophosphate (1.42 mL, 11.93 mmol) in an ice-water bath, followed by dropwise addition of triethylamine (6.63 mL, 47.72 mmol), and the reaction mixture was stirred for 3 hours at this temperature. Then 4-nitrophenol (1660 mg, 11.93 mmol) and triethylamine (6.63 mL, 47.72 mmol) were added and the reaction mixture was slowly warmed to room temperature and stirred at this temperature overnight. The reaction mixture was used directly for the next step without treatment. LC-MS (ESI): m/z 361.2 (M+H)⁺.

### Synthesis of Compound 109

At room temperature, a portion of the above reaction mixture 109-a (about 0.56 mmol) was added acetonitrile (5 mL), then compound 4 (163 mg, 0.28 mmol) and DBU (166 uL, 1.11 mmol) were added and the mixture was stirred at room temperature under protection of nitrogen overnight. The reaction mixture was concentrated to dryness by rotary evaporation at room temperature, purified by prep-HPLC (ammonium bicarbonate) and lyophilized to give compound 109 (143 mg, ca. 64%).

LC-MS (ESI): m/z 810.3 (M+H)⁺; ¹H NMR (400M, DMSO-d₆): δ 10.27 (1H, s), 8.23-8.14 (1H, m), 8.15-6.90 (1H, m), 8.03-7.91 (1H, m), 7.62 (1H, t, J=9.2 Hz), 7.58-7.49 (1H, m), 6.15-5.98 (1H, m), 5.95-5.84 (1H, m), 5.31 (1H, d, J=54.4 Hz), 4.91=4.77 (1H, m), 4.91-3.94 (6H, m), 3.90-3.75 (1H, m), 3.20-2.97 (5H, m), 2.90-2.79 (1H, m), 2.21-1.58 (12H, m), 1.33-1.04 (12H, m).

### Synthetic Route of Compound 110

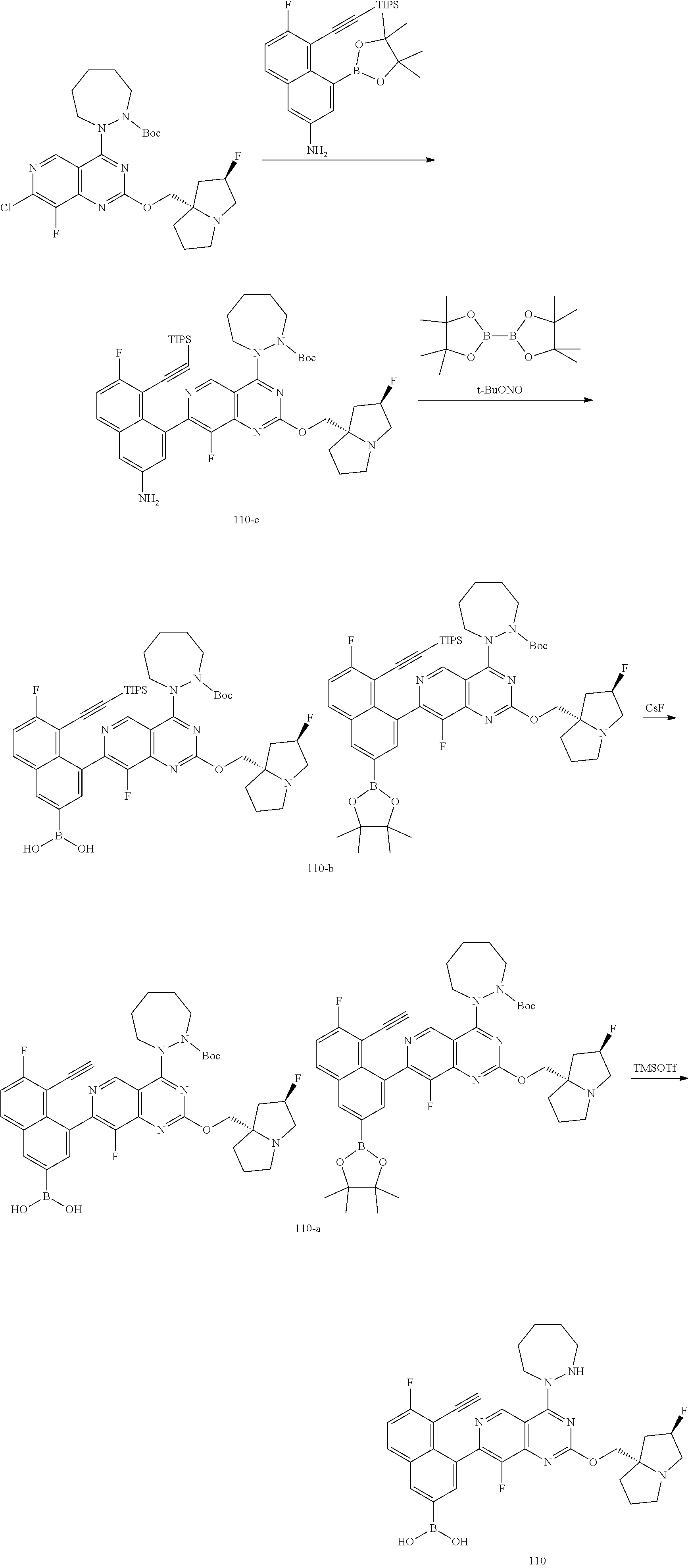

### Synthesis of Compound 110-c

Compound 3-b (200 mg, 0.37 mmol) was dissolved in THF (5 mL) in a microwave tube at room temperature, followed by addition of 6-fluoro-4-(4,4,5,5-tetramethyl-1,3,2-dioxaborolan-2-yl)-5-((tris-isopropylsilyl)ethynyl)naphthalen-2-amine (208 mg, 0.45 mmol), methylsulfonato (diadamantyl-n-butylphosphino)-2′-amino-1,1′-biphenyl-2-yl)palladium(II) (54 mg, 0.074 mmol), potassium phosphate (236 mg, 1.11), and water (1 mL). The reaction mixture was stirred overnight at 65° C. under protection of nitrogen. After cooled to room temperature, the reaction mixture was added 100 mL of water, extracted with ethyl acetate (100 mL). The organic phase was dried over anhydrous sodium sulfate, filtered, and the filtrate was concentrated at reduced pressure, and the residue was purified by a flash column chromatography (methanol/dichloromethane=0-10%) to give compound 110-c (303 mg, 97%). LC-MS (ESI): m/z=844.5 [MS+H]⁺.

### Synthesis of Compound 110-b

Compound 110-c (200 mg, 0.24 mmol) was dissolved in anhydrous acetonitrile (50 mL) at room temperature, followed by addition of compound pinacol ester of bisboronic acid (602 mg, 2.37 mmol), tert-butyl nitrite (199 uL, 1.66 mmol). The reaction mixture was stirred at 60° C. under argon for 6 hours. The reaction mixture was cooled to room temperature, concentrated at reduced pressure, and the residue was purified by a flash column chromatography (MeOH/DCM=0-10%) to afford compound 110-b (126 mg, crude, mixture), and the crude was used directly in the next step. LC-MS (ESI): m/z=873.5[M+H]⁺; m/z=955.6[M+H]⁺.

### Synthesis of Compound 110-a

Compound 110-b (126 mg, 0.13 mmol as borate ester) was dissolved in DMF (5 mL) at room temperature, cesium fluoride (401 mg, 2.64 mmol) was added and the reaction mixture was stirred at room temperature under protection of nitrogen for 2 hours. The reaction was quenched by adding water (50 mL), extracted with ethyl acetate (100 mL). The organic phase was washed with brine (100 mL*5), dried over anhydrous sodium sulfate, filtered, and the filtrate was concentrated at reduced pressure and dried under vacuum to give compound 110-a (100 mg, crude mixture), and the crude was used directly in the next step. LC-MS (ESI): m/z=717.4[M+H]⁺; m/z=799.5[M+H]⁺.

### Synthesis of Compound 110

Compound 110-a (100 mg, 0.13 mmol, as borate ester) was dissolved in DCM (5 mL) at room temperature, trimethylsilyl trifluoromethanesulfonate (500 uL, 2.75 mmol) was added in an ice-water bath and the reaction mixture was stirred under protection of nitrogen for 1 hr in an ice-water bath. The reaction mixture was added 10% NaHSO₄ aqueous solution (5 mL), methanol (5 mL), then was stirred at room temperature for 60 min. It was added saturated sodium bicarbonate solution (50 mL), extracted with DCM (100 mL), dried over anhydrous sodium sulfate, filtered, and the filtrate was concentrated at reduced pressure, and the residue was purified by Prep-HPLC (basic method, ammonium bicarbonate) to give compound 110 (2 mg, 2.5%). LC-MS (ESI): m/z=617.3[M+H]⁺; ¹H NMR (DMSO-d₆, 400 MHz): δ 10.27 (1H, s), 8.57 (1H, s), 8.41 (2H, s), 8.23-8.20 (1H, m), 7.98 (1H, s), 7.58 (1H, t, J=9.2 Hz), 5.86 (1H, t, J=6.0 Hz), 5.28 (1H, d, J=54.0 Hz), 4.20-3.88 (5H, m), 3.14-2.96 (5H, m), 2.87-2.78 (1H, m), 2.15-1.58 (12H, m).

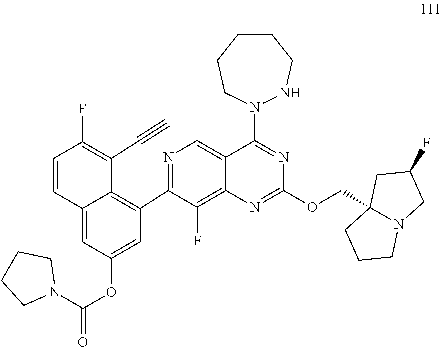 [SMILES: C#Cc1c(F)ccc2cc(OC(=O)N3CCCC3)cc(-c3ncc4c(N5CCCCCN5)nc(OC[C@@]56CCCN5C[C@H](F)C6)nc4c3F)c12]

Referring to the synthetic route of compound 89, compound 111 was obtained by using 1-pyrrolidinecarbonyl chloride instead of dimethylcarbamoyl chloride. LC-MS (ESI): m/z=686.5(M+H)+¹H NMR (400 MHz, DMSO-d₆): δ 10.26 (1H, s), 8.25-8.16 (1H, m), 7.98 (1H, d, J=2.4 Hz), 7.62 (1H, t, J=9.2 Hz), 7.49 (1H, d, J=2.4 Hz), 5.88 (1H, t, J=6.0 Hz), 5.28 (1H, d, J=53.6 Hz), 4.20-3.93 (5H, m), 3.56 (2H, t, J=6.4 Hz), 3.37 (2H, t, J=6.8 Hz), 3.53-2.95 (5H, m), 2.88-2.75 (1H, m), 2.22-1.83 (10H, m), 1.82-1.59 (6H, m).

### Synthesis of Compound 112

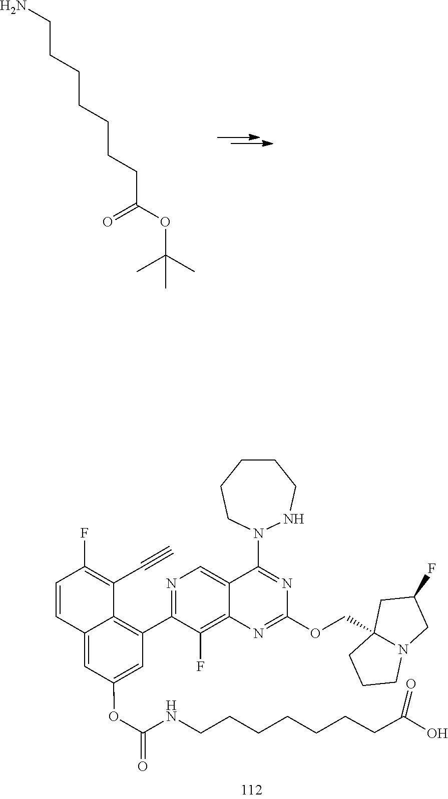 [SMILES: CC(C)(C)OC(=O)CCCCCCCN ; C#Cc1c(F)ccc2cc(OC(=O)NCCCCCCCC(=O)O)cc(-c3ncc4c(N5CCCCCN5)nc(OC[C@@]56CCCN5C[C@H](F)C6)nc4c3F)c12]

Referring to the synthetic route of compound 100, tert-butyl 8-aminooctanoate was used instead of tert-butyl 4-aminobutyrate to obtain compound 112. LC-MS (ESI): m/z=774.4(M+H)⁺.

### Synthetic Route of Compound 113

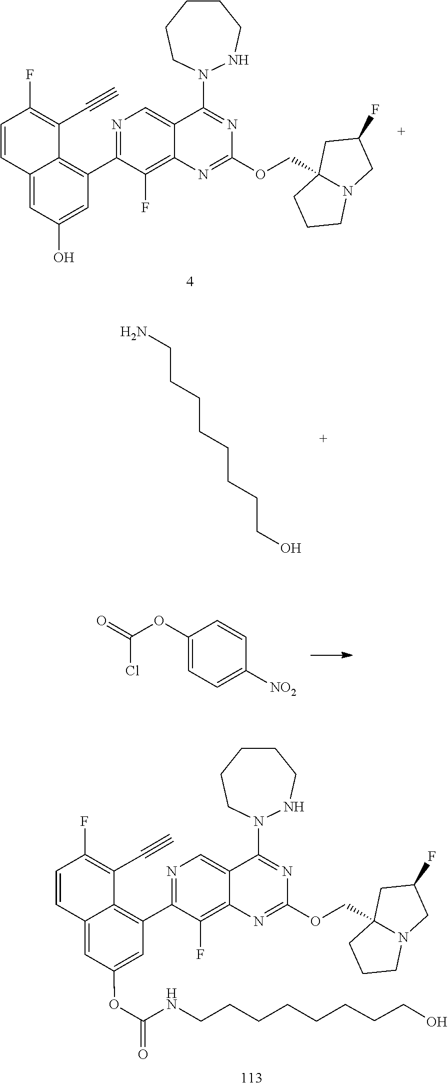 [SMILES: C#Cc1c(F)ccc2cc(O)cc(-c3ncc4c(N5CCCCCN5)nc(OC[C@@]56CCCN5C[C@H](F)C6)nc4c3F)c12 ; NCCCCCCCCO ; O=C(Cl)Oc1ccc([N+](=O)[O-])cc1 ; C#Cc1c(F)ccc2cc(OC(=O)NCCCCCCCCO)cc(-c3ncc4c(N5CCCCCN5)nc(OC[C@@]56CCCN5C[C@H](F)C6)nc4c3F)c12]

### Synthesis of Compound 113

To a reaction flask were added compound 4 (100 mg, 0.17 mmol), p-nitrophenyl chloroformate (41 mg, 0.20 mmol), DIPEA (66 mg, 0.51 mmol) and dichloromethane (10 mL), respectively. The mixture was stirred for 1 hr under protection of nitrogen in an ice-water bath, then was added 8-Amino-1-octanol (25 mg, 0.17 mmol) and stirred in an ice-water bath under protection of nitrogen for 2 hours. The reaction mixture was concentrated and the residue was purified by prep-HPLC to give compound 113 (49 mg, 38%). LC-MS (ESI): m/z=760.6(M+H)⁺.

### Synthesis of Compound 114

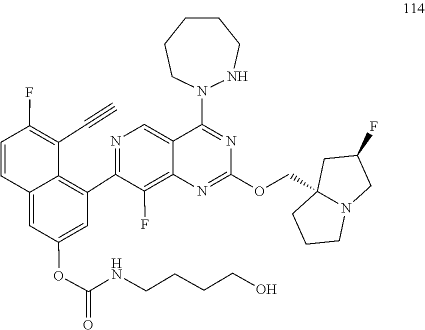 [SMILES: C#Cc1c(F)ccc2cc(OC(=O)NCCCCO)cc(-c3ncc4c(N5CCCCCN5)nc(OC[C@@]56CCCN5C[C@H](F)C6)nc4c3F)c12]

Referring to the synthetic route of compound 113, compound 114 was obtained using 4-amino-1-butanol instead of 8-amino-1-octanol. LC-MS (ESI): m/z=704.3(M+H)⁺; ¹H NMR (400 MHz, DMSO-d₆): δ 10.27 (1H, s), 8.26-8.15 (1H, m), 8.08-7.93 (2H, m), 7.62 (1H, t, J=8.8 Hz), 7.51-7.41 (1H, m), 5.89 (1H, t, J=6.0 Hz), 5.29 (1H, d, J=54.0 Hz), 4.42 (1H, t, J=4.8 Hz), 4.23-3.90 (4H, m), 4.03 (1H, s), 3.53-3.35 (2H, m), 3.18-2.95 (7H, m), 2.23-1.78 (9H, m), 1.77-1.59 (4H, m), 1.58-1.39 (4H, m).

### Synthetic Route of Compound 115

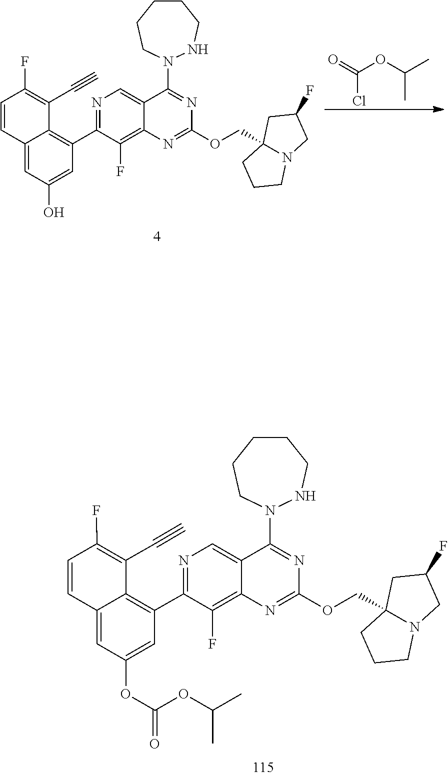

### Synthesis of Compound 115

To a reaction flask were added compound 4 (100 mg, 0.17 mmol), isopropyl chloroformate (25 mg, 0.20 mmol), DIPEA (66 mg, 0.51 mmol), dichloromethane (20 mL), respectively, and the resulting mixture was stirred in an ice-water bath under protection of nitrogen for 1 hour. The reaction mixture was concentrated and the residue was purified by column chromatography (MeOH/DCM=0/100;1/10) to obtain compound 115 (57 mg, 50%). LC-MS (ESI): m/z=675.8(M+H)⁺.

### Synthetic Route of Compound 116

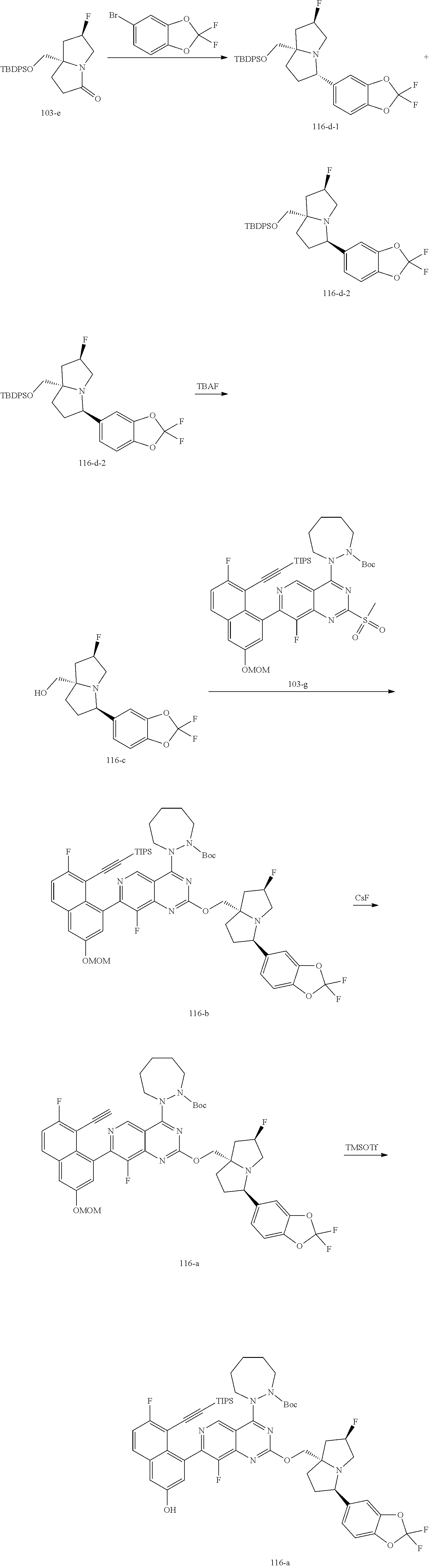

### Synthesis of Compounds 116-d-1 and 116-d-2

In an ice-water bath and under protection of nitrogen, a solution of isopropylmagnesium chloride-lithiumchloride in THF (1.3 M, 3.93 mL, 5.10 mmol) was added dropwise to a solution of 5-bromo-2,2-difluoro-1,3-benzodioxole (1152 mg, 4.86 mmol) in anhydrous THF (10 mnL) after degassed and purged with nitrogen. The reaction was allowed to stirred at room temperature for 1 h to obtain solution A. To a reaction vial were added 103-e (1000 mg, 2.43 mmol), toluene (12 mL), and carbonylbis(triphenylphosphine)iridium(I) chloride (50 mg, 0.06 mmol). After dissolved and degassed and purged with nitrogen, the mixture was added 1,1,3,3-tetramethyldisiloxane (490 mg, 3.65 mmol) dropwise in an ice-water bath. After the dropwise addition, the reaction mixture was stirred in an ice-water bath for 30 min and cooled down to −78° C. with a dry ice-acetone bath, was slowly added the solution A dropwise. After the dropwise addition, the dry ice-acetone bath was removed and the reaction was naturally warmed for 2 hours. The reaction mixture was quenched with saturated aqueous ammonium chloride in an ice-water bath and the aqueous phase was extracted with EA. The organic phases were combined, washed with water, dried over anhydrous sodium sulfate, concentrated, and the residue was purified by column chromatography (mobile phase: PE/EA 100/0 to 95/5) to give compounds 116-d-1 (560 mg, 42%) and 116-d-2 (650 mg, 48%). LC-MS (ESI): m/z 554.4 (M+H)⁺.

### Synthesis of Compound 116-c

116-d-2 (650 mg, 1.17 mmol) and THF (20 mL) were added to a reaction vial at room temperature. After degassed and purged with nitrogen, the mixture was added tetrabutylammonium fluoride (556 mg, 1.76 mmol) dropwise, and the reaction mixture was stirred at 50° C. for 2 hours. The reaction mixture was cooled to room temperature, was added saturated aqueous sodium bicarbonate until it was basic, and extracted three times with DCM/MeOH=10/1. The organic phases were combined, dried, concentrated and the residue was purified by column chromatography (mobile phase: DCM/MeOH 10/0 to 10/1) to give compound 116-c (280 mg, 76%). LC-MS (ESI): m/z 316.2 (M+H)⁺.

### Synthesis of compound 116-b

103-g (642 mg, 0.79 mmol), toluene (20 mL) and 116-c (250 mg, 0.79 mmol) were added to a reaction vial at room temperature. After degassed and purged with nitrogen, cooled down to 0° C., the mixture was added sodium tert-butoxide (229 mg, 2.38 mmol), and was kept at 0° C. for 1 hour. The reaction mixture was mixed with silica gel and purified by column chromatography (mobile phase: PE/EA 10/0 to 6/4) to give compound 116-b (760 mg, 92%). LC-MS (ESI): m/z 1046.0 (M+H)⁺.

### Synthesis of Compound 116-a

116-b (760 mg, 0.73 mmol), DMF (6 mL) and cesium fluoride (1104 mg, 7.27 mmol) were added to a reaction flask at room temperature. After degassed and purged with nitrogen, the mixture was stirred at 50° C. for 1 h. The reaction mixture was added water and the aqueous phase was extracted with EA. The organic phases were combined, washed with water, brine, dried over anhydrous sodium sulfate, and concentrated to give compound 116-a (640 mg, 99%). LC-MS (ESI): m/z 889.8 (M+H)⁺.

### Synthesis of Compound 116

116-a (640 mg, 0.72 mmol) and DCM (60 mL) were added to a reaction vial at room temperature. After degassed and purged with nitrogen and cooled down to 0° C., the mixture was was added trimethylsilyl trifluoromethanesulfonate (6 mL) dropwise and stirred for 3 hours. It was added 10% aqueous sodium bisulfate (50 mL) and stirred for 1 hour. The reaction mixture was extracted with DCM. The organic phases were combined, washed with saturated aqueous sodium bicarbonate, washed with water, brine, dried, concentrated, and the residue was purified by column chromatography (mobile phase: DCM/MEOH 10/0 to 10/1) to give compound 116 (255 mg, 48%).

LC-MS (ESI): m/z 745.4 (M+H)⁺; ¹H NMR (400 MHz, CDCl₃): δ 10.29-10.02 (1H, d, J=47.2 Hz), 7.52-7.21 (2H, m), 7.20-6.94 (4H, m), 6.93-6.77 (1H, m), 5.25-4.90 (1H, d, J=54.8 Hz), 4.64-3.81 (5H, m), 3.26-2.66 (4H, m), 2.54-1.46 (13H, m).

### Synthetic Route of Compound 117

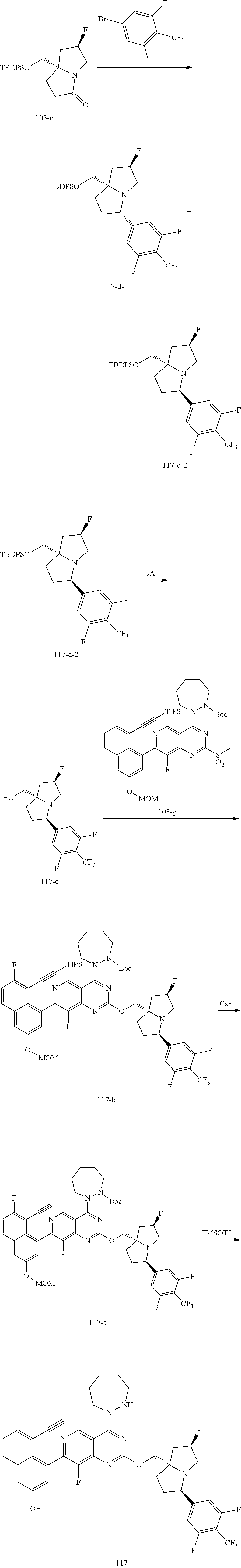

### Synthesis of Compounds 117-d-1 and 117-d-2

To a solution of 4-bromo-2,6-difluorobenzotrifluoride (634 mg, 2.43 mmol) in anhydrous tetrahydrofuran (10 mL) in an ice-ethanol bath was slowly added isopropylmagnesium chloride (1.276 mL, 2.55 mmol) dropwise. The reaction mixture was stirred at this temperature for 1 hour and then allowed to stand still to obtain solution A.

To a reaction flask were added 103-e (500 mg, 1.21 mmol), carbonylbis(triphenylphosphine)iridium(I) chloride (95 mg, 0.12 mmol) and anhydrous toluene (12 mL), followed by slow and dropwise addition of 1,1,3,3-tetramethyldisiloxane (0.32 mL, 1.82 mmol) in an ice-water bath. The reaction mixture was stirred in an ice-water bath for 35 min and then cooled to −78° C., was added the solution A dropwise slowly. After addition, the reaction mixture was held at −78° C. for 30 min. After removed low-temperature bath, the reaction mixture was slowly warmed to room temperature and stirred for 45 min. The reaction was quenched by the addition of saturated ammonium chloride solution in an ice-water bath, added water and a small amount of anhydrous solid sodium sulfate, and extracted once with ethyl acetate/petroleum ether (4:1). The organic phase was dried over anhydrous sodium sulfate, filtered, evaporated, and the residue was purified by column chromatography (mobile phase: petroleum ether/ethyl acetate, 100/0 to 95/5) to give compound 117-d-1 (185 mg, 26%), LC-MS (ESI): m/z 578.4 (M+H)⁺; compound 117-d-2 (172 mg, 25%), LC-MS (ESI): m/z 578.4 (M+H)*.

### Synthesis of Compound 117-c

To a solution of 117-d-2 (210 mg, 0.364 mmol) in tetrahydrofuran (10 mL) was added a solution of tetrabutylammonium fluoride in tetrahydrofuran solution (1M, 0.55 mL, 0.55 mmol) and stirred at 50° C. for 2 hours under protection of nitrogen. Concentrated to dryness by rotary evaporation and the residue was purified by column chromatography (mobile phase: methanol/dichloromethane, 0-10%) to obtain 117-c (60 mg, 50%). LC-MS (ESI): m/z 340.1 (M+H)⁺.

### Synthesis of Compound 117-b

To a reaction flask were added 117-c (60 mg, 0.18 mmol), 103-g (143 mg, 0.18 mmol) and toluene (8 mL). Cooled in an ice-water bath, the above mixture was added sodium tert-butoxide (68 mg, 0.71 mmol) and stirred for 1 hour. The reaction mixture was directly purified by column chromatography (mobile phase: ethyl acetate/petroleum ether, 0-30%) to afford 117-b (140 mg, 74%). LC-MS (ESI): m/z 1069.5 (M+H)⁺.

### Synthesis of Compound 117-a

To a solution of 117-b (140 mg, 0.13 mmol)in N,N-dimethylformamide (6 mL) was added cesium fluoride (398 mg, 2.61 mmol) at room temperature and the mixture was stirred for 3 hours at room temperature. The reaction mixture was poured water and extracted twice with ethyl acetate. The organic phase was washed three times with brine, concentrated to dryness by rotary evaporation, and the residue was purified by column chromatography (mobile phase: ethyl acetate/petroleum ether, 0-50%) to give 117-a (109 mg, 91%). LC-MS (ESI): m/z 913.4 (M+H)⁺.

### Synthesis of Compound 117

TMSOTf (0.22 mL, 1.19 mmol) was added to a solution of 117-a (109 mg, 0.12 mmol) in dichloromethane (5 mL) in an ice-water bath. The reaction mixture was stirred at this temperature for 2 hours, then was added 10 mL of 10% sodium bisulfate solution, sonicated, stirred for 30 min to disperse uniformly, then adjusted pH to basic with saturated sodium bicarbonate solution, extracted with dichloromethane (30 mL*2), the combined organic phases were dried over anhydrous sodium sulfate, filtered, concentrated to dryness by rotary evaporation, and the residue was purified by column chromatography (mobile phase: MeOH/DCM, 0-4%) to afford 117 (48 mg, 52%). LC -MS (ESI): m/z 769.4 (M+H)⁺.

### Synthetic Route of Compound 118

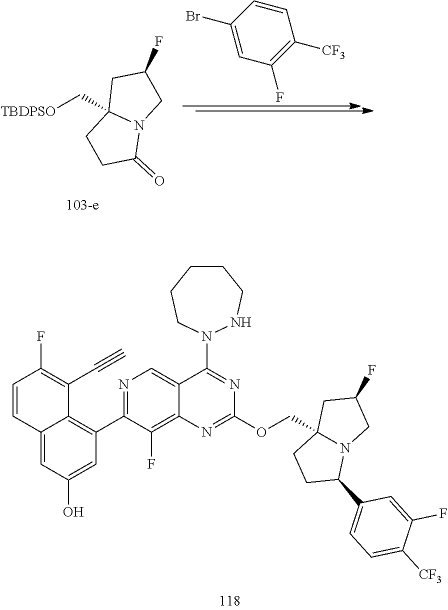

Referring to the synthetic route of compound 117, 4-bromo-2-fluorobenzotrifluoride was used instead of 4-bromo-2,6-difluorobenzotrifluoride to obtain compound 118. LC-MS (ESI): m/z 751.3 (M+H)⁺.

### Synthetic Route of Compound 119

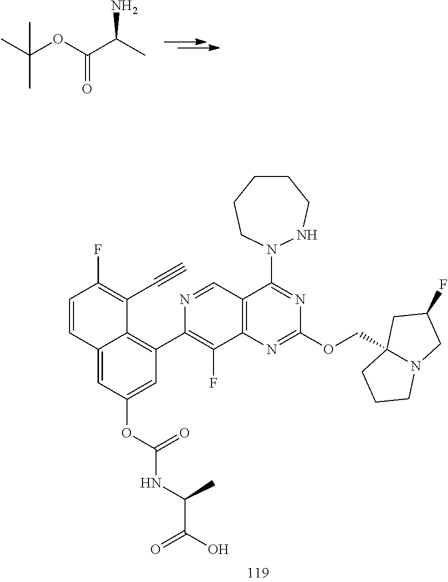

Referring to the synthetic route of compound 100, tert-butyl L-alanine was used instead of tert-butyl 4-aminobutyrate to obtain compound 119. LC-MS (ESI): m/z 704.3 (M+H)⁺.

### Synthetic Route of Compound 120

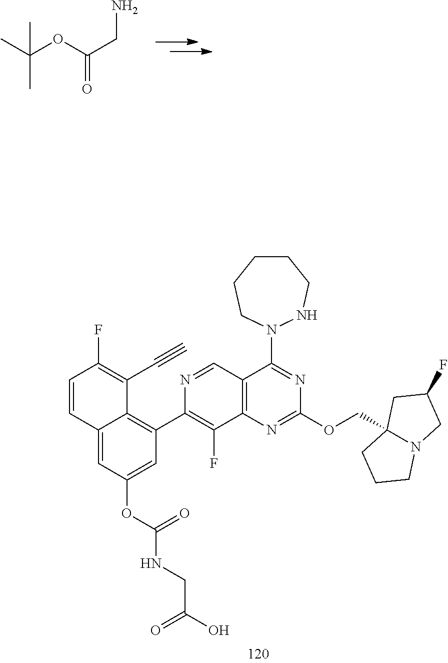

Referring to the synthetic route of compound 100, tert-butyl glycinate was used instead of tert-butyl 4-aminobutyrate to obtain compound 120. LC-MS (ESI): m/z 690.3 (M+H)⁺; ¹H NMR (400M, DMSO-d₆): δ 10.24 (1H, s), 8.38 (1H, s), 8.00-7.92 (1H, m), 7.46 (1H, t, J=9.2 Hz), 7.38 (1H, d, J=2.4 Hz), 7.17 (1H, d, J=2.4 Hz), 5.87(1H, t, J=6.0 Hz), 5.28 (1H, d, J=54.4 Hz), 4.15-3.94 (4H, m), 3.91 (1H, s), 3.49-3.21 (5H, m), 3.15-2.96 (5H, m), 2.88-2.77 (1H, m), 2.17-1.55 (12H, m).

### Synthesis of Compound 121

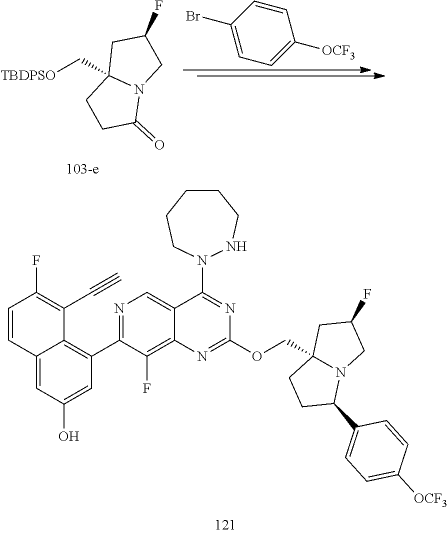

Referring to the synthetic route of compound 117, compound 121 was obtained using p-bromotrifluoromethoxybenzene instead of 4-bromo-2,6-difluorobenzotrifluoride. LC-MS (ESI): m/z=749.4(M+H)⁺.

### Synthetic Route of Compound 122

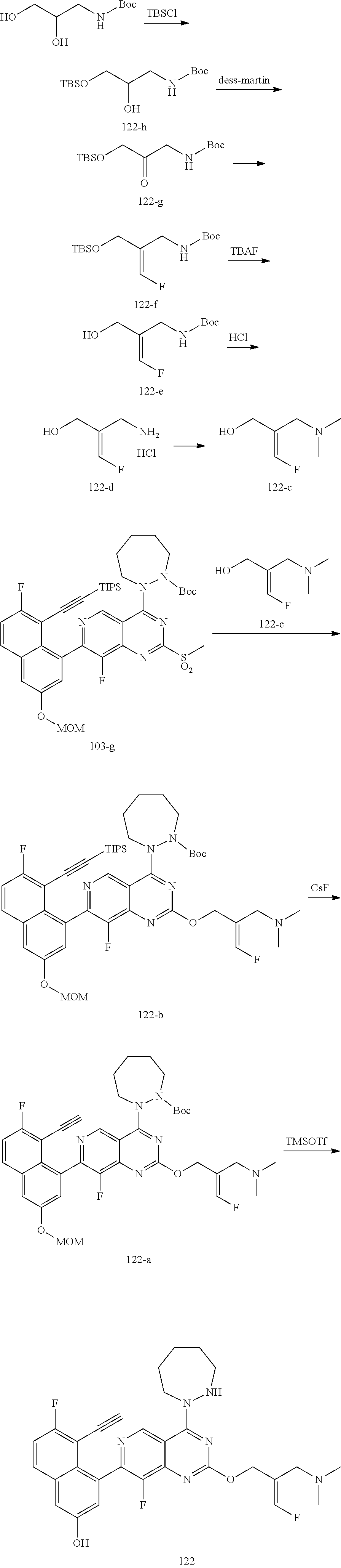

### Synthesis of Compound 122-h

Tert-butyl N-(2,3-dihydroxypropyl)carbamate (5 g, 26.15 mmol), dichloromethane (70 mL) and imidazole (3.56 g, 52.29 mmol) were added to a reaction flask, followed by addition of tert-butyldimethylchlorosilane (4.18 g, 27.72 mmol) cooled in an ice-water bath, and the reaction mixture was stirred for 2 hours at room temperature under protection of nitrogen. Quenched with saturated sodium bicarbonate solution, extracted twice with dichloromethane, concentrated to dryness by rotary evaporation and the residue was purified by column chromatography (mobile phase: methanol/dichloromethane, 0-5%) to give compound 122-h (6.9 g, 86%). LC-MS (ESI): m/z 306.2 (M+H)⁺.

### Synthesis of Compound 122-g

122-h (2 g, 6.55 mmol), dichloromethane (50 mL) and Dess-Martin oxidizer (3.61 g, 8.51 mmol) were added to a reaction vial and the reaction mixture was stirred at room temperature overnight under protection of nitrogen. On the next day, it was quenched by adding saturated sodium bicarbonate solution and saturated sodium sulfite solution, extracted twice with dichloromethane, concentrated to dryness by rotary evaporation and the residue was purified by column chromatography (mobile phase: ethyl acetate/petroleum ether, 0-20%) to give compound 122-g (1.53 g, 77%). LC-MS (ESI): m/z 248.1 (M+H-56)⁺.

### Synthesis of Compound 122-f

To a reaction flask were added (fluoromethyl)tetrafluoroborate triphenylphosphate (2.80 g, 7.32 mmol) and tetrahydrofuran (60 mL). Cooled in a dry ice-acetone bath, the above mixture was added sodium bis(trimethylsilyl)amide (7.32 mL, 7.32 mmol) dropwise. After addition, the reaction mixture was stirred at this temperature for 1 h, then was added a solution of 122-g (1.48 g, 4.88 mmol) in tetrahydrofuran (10 mL) dropwise. After addition, the reaction mixture was naturally warmed to room temperature and stirred for 1 hour. The reaction mixture was quenched with water and the aqueous phase was extracted twice with ethyl acetate. The organic phase was washed with brine, dried, concentrated to dryness by rotary evaporation, and the residue was purified by column chromatography (mobile phase: ethyl acetate/petroleum ether, 0-5%) to give compound 122-f (1.2 g, 77%). LC-MS (ESI): m/z 320.2 (M+H)⁺.

### Synthesis of Compound 122-e

122-f (1.2 g, 3.76 mmol), tetrahydrofuran (10 mL), tetrabutylammonium fluoride tetrahydrofuran solution (9.39 mL, 9.39 mmol) were added to a reaction flask and stirred at 50° C. for 2 hrs under protection of nitrogen. The reaction mixture was cooled to room temperature, was added water and extracted twice with ethyl acetate. The organic phase was washed with brine, concentrated to dryness by rotary evaporation and the residue was purified by column chromatography (mobile phase: ethyl acetate/petroleum ether, 0-50%) to give compound 122-e (626 mg, 81%). LC-MS (ESI): m/z 206.3 (M+H)⁺.

### Synthesis of Compound 122-d

122-e (626 mg, 3.05 mmol), acetonitrile (10 mL), hydrochloride/1,4-dioxane (5 mL, 20 mmol) were added to a reaction vial and the mixture was stirred at room temperature for 1 hr under protection of nitrogen. It was concentrated to dryness by rotary evaporation to give compound 122-d (320 mg, 100%), which was used directly in the next step without purification. LC-MS (ESI): m/z 106.3 (M+H)⁺.

### Synthesis of Compound 122-c

122-d (100 mg, 0.95 mmol), methanol (20 mL) and paraformaldehyde (514 mg, 5.71 mmol) were added to a reaction vial. After stirred for 30 min, the above mixture was added sodium cyanoborohydride (359 mg, 5.71 mmol) and stirred at room temperature overnight. The next day, it was concentrated to dryness by rotary evaporation and the residue was purified by column chromatography (mobile phase: ammonia methanol/dichloromethane, 0-10%) to give compound 122-c (135 mg, 107%). LC-MS (ESI): m/z 134.3 (M+H)⁺.

### Synthesis of Compound 122-b

To a reaction vial were added 122-c (25 mg, 0.18 mmol), 103-g (100 mg, 0.12 mmol) and toluene (8 mL). Cooled in an ice-water bath, the above mixture was added sodium tert-butoxide (47 mg, 0.49 mmol) and stirred for 1 hour. The reaction mixture was directly purified by column chromatography (mobile phase: methanol/dichloromethane, 0-5%) to give 122-b (75 mg, 70%). LC-MS (ESI): m/z 863.5 (M+H)⁺.

### Synthesis of Compound 122-a

To a solution of 122-b (75 mg, 0.087 mmol) in N,N-dimethylformamide (6 mL) was added cesium fluoride (264 mg, 1.74 mmol) at room temperature and the mixture was stirred for 2 h at room temperature. The reaction mixture was poured water, extracted twice with ethyl acetate, washed three times with brine, dried over anhydrous sodium sulfate, and concentrated to dryness by rotary evaporation to give 122-a (51 mg, 83%), which was used directly in the next step without purification.

LC-MS (ESI): m/z 707.6 (M+H)⁺.

### Synthesis of Compound 122

TMSOTf (0.39 mL, 2.17 mmol) was added to a solution of 122-a (51 mg, 0.072 mmol) in dichloromethane (5 mL) in an ice-water bath. The reaction mixture was stirred at this temperature for 1 hour, then was added 10 mL of 10% sodium bisulfate solution, sonicated, and stirred for 30 minutes to make a homogeneous dispersion, then adjusted pH to basic with saturated sodium bicarbonate solution, extracted with dichloromethane (30 mL*3), and the combined organic phases were dried over anhydrous sodium sulfate, filtered, concentrated to dryness by rotary evaporation, and the residue was purified by prep-HPLC (ammonium bicarbonate) to give 122 (17 mg, 42%).

LC-MS (ESI): m/z 563.3 (M+H)⁺; ¹H NMR (400 M, CDCl₃): δ 10.19 (1H, s), 7.61-7.48 (1H, m), 7.25-6.84 (3H, m), 5.08-4.84 (2H, m), 4.49-4.35 (1H, m), 4.10-3.90 (2H, m), 3.45-3.25 (2H, m), 3.20-3.00 (2H, m), 2.49-2.32 (6H, m), 2.04-1.87 (3H, m), 1.80-1.58 (5H, m).

### Synthetic Route of Compound 123

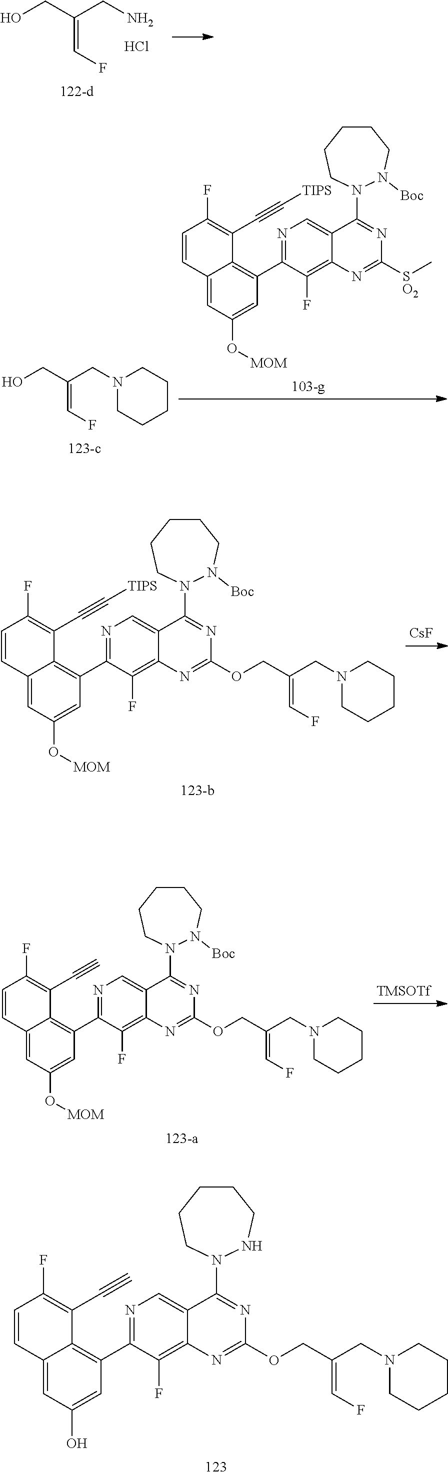

### Synthesis of Compound 123-c

To a reaction flask were added 122-d (70 mg, 0.39 mmol), acetonitrile (10 mL), 1,5-dibromopentane (161 uL, 1.18 mmol) and potassium carbonate (435 mg, 3.15 mmol). The reaction mixture was stirred at room temperature for 2 h, then was supplemented with 1,5-dibromopentane (161 uL, 1.18 mmol) and continued stirring overnight. On the next day, the reaction mixture was filtered and the filtrate was used as a solution of compound 123-c (68 mg, 100%), which was directly used in the next step. LC-MS (ESI): m/z 174.2 (M+H)⁺.

### Synthesis of Compound 123-b

Cooled in an ice-water bath, a reaction vial was added 103-g (150 mg, 0.19 mmol), toluene (15 mL), a solution of 123-c (64 mg, 0.37 mmol) in acetonitrile and sodium tert-butoxide (142 mg, 1.48 mmol) and the resulting mixture was stirred for 1 hr. It was quenched with water, extracted twice with ethyl acetate, washed with brine, dried over anhydrous sodium sulfate, concentrated to dryness by rotary evaporation, and the residue was purified by column chromatography (mobile phase: methanol/dichloromethane, 0-10%) to give 123-b (74 mg, 44%). LC-MS (ESI): m/z 903.5 (M+H)⁺.

### Synthesis of compound 123-a

To a solution of 123-b (74 mg, 0.082 mmol) in N,N-dimethylformamide (6 mL) was added cesium fluoride (249 mg, 1.64 mmol) at room temperature, and the mixture was stirred for 1 h at room temperature. The reaction mixture was poured water, extracted twice with ethyl acetate, washed three times with brine, dried over anhydrous sodium sulfate, and concentrated to dryness by rotary evaporation to give 123-a (61 mg, 100%), which was used directly in the next step without purification. LC-MS (ESI): m/z 747.8 (M+H)⁺.

### Synthesis of Compound 123

TMSOTf (0.33 mL, 1.80 mmol) was added to a solution of 123-a (61 mg, 0.082 mmol) in dichloromethane (5 mL) in an ice-water bath. The reaction mixture was stirred at this temperature for 1 hour. It was added methanol and continued stirring for 1 h. The solution was concentrated to dryness by rotary evaporation and the residue was purified by prep-HPLC (ammonium bicarbonate) to give 123 (10 mg, 20%). LC-MS (ESI): m/z 603.3 (M+H)⁺.

### Synthetic Route of Compound 124

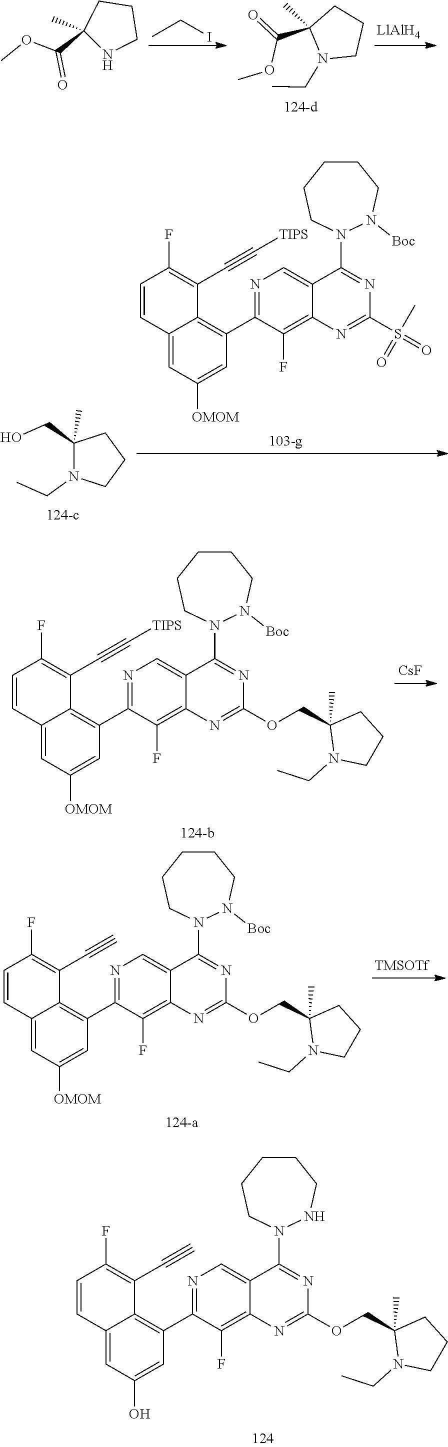

### Synthesis of Compound 124-d

Potassium carbonate (2.316 g, 16.76 mmol) and ethyl iodide (1.742 g, 11.17 mmol) were added to a solution of compound (R)-methyl 2-methylpyrrolidine-2-carboxylic acid methyl ester (800 mg, 5.59 mmol) in acetonitrile (30 mL), then the reaction mixture was stirred for 12 h at 90° C. It was filtered through celite and the filtrate was concentrated at reduced pressure to obtian the crude product, which was purified by column chromatography (mobile phase, PE/EA 3/1) to obtain compound 124-d (450 mg, 47%). LC-MS (ESI): m/z 172.3(M+H)⁺.

### Synthesis of Compound 124-c

Compound 124-d (450 mg, 2.63 mmol) was dissolved in anhydrous tetrahydrofuran (10 mL). After degassed and purged with nitrogen for 3 times, it was added a solution of tetrahydroaluminum lithium in tetrahydrofuran (1 M, 2.89 mL, 2.89 mmol) dropwise in an ice-water bath. The reaction mixture was stirred in an ice-water bath for 1 h, then it was added 1 g of sodium sulfate decahydrate, the mixture was stirred for 10 min, then filtered through celite, and the filtrate was concentrated at reduced pressure and dried to give the crude product 124-c (120 mg, 32%). LC-MS (ESI): m/z 144.3(M+H)⁺.

### Synthesis of Compound 124-b

Compounds 103-g (100 mg, 0.12 mmol) and 124-c (44 mg, 0.31 mmol) were dissolved in dichloromethane (6 mL). Cooled in an ice-water bath, the above mixture was added sodium tert-butanol (24 mg, 0.25 mmol), and stirred for 1 hr in an ice-water bath, then was added water (50 mL), and it was extracted with ethyl acetate (50 mL), washed with brine (50 mL) and dried over sodium sulfate. The desiccant was filtered off and the filtrate was concentrated at reduced pressure to obtain the crude product, which was purified by column chromatography (mobile phase, DCM/MeOH 20/1) to obtain compound 124-b (85 mg, 79%). LC-MS (ESI): m/z 873.6 (M+H)⁺.

### Synthesis of Compound 124-a

Compound 124-b (85 mg, 0.097 mmol) was dissolved in N,N-dimethylformamide (8 mL), to which cesium fluoride (89 mg, 0.59 mmol) was added, and the reaction mixture was stirred at room temperature for 12 h. After stirred for 12 h, the reaction mixture was added water (100 mL), extracted with ethyl acetate (50 mL), washed with brine (50 mL), dried over sodium sulfate. The desiccant was filtered off and the filtrate was concentrated at reduced pressure to obtain the crude product, which was purified by column chromatography (mobile phase, DCM/MeOH 20/1) to obtain compound 124-a (50 mg, 76%). LC-MS (ESI): m/z 717.5 (M+H)⁺.

### Synthesis of Compound 124

Compound 124-a (50 mg, 0.070 mmol) was dissolved in dichloromethane (4 mL), to which tert-butyldimethylsilyl trifluoromethanesulfonate (0.4 mL) was added in an ice-water bath, and the reaction mixture was stirred for 2 hr at room temperature. It was added 10% sodium bisulfate solution (3 mL) and continued stirring for 2 hr, neutralized with saturated sodium bicarbonate solution, extracted with dichloromethane (50 mL), washed with brine (50 mL) and dried over sodium sulfate. The desiccant was filtered off and the filtrate was concentrated at reduced pressure to obtain the crude product, which was purified by prep-HPLC (basic conditions) to give compound 124 (10 mg, 25%). LC-MS (ESI): m/z 573.3 (M+H)⁺.

### Synthetic Route of Compound 125

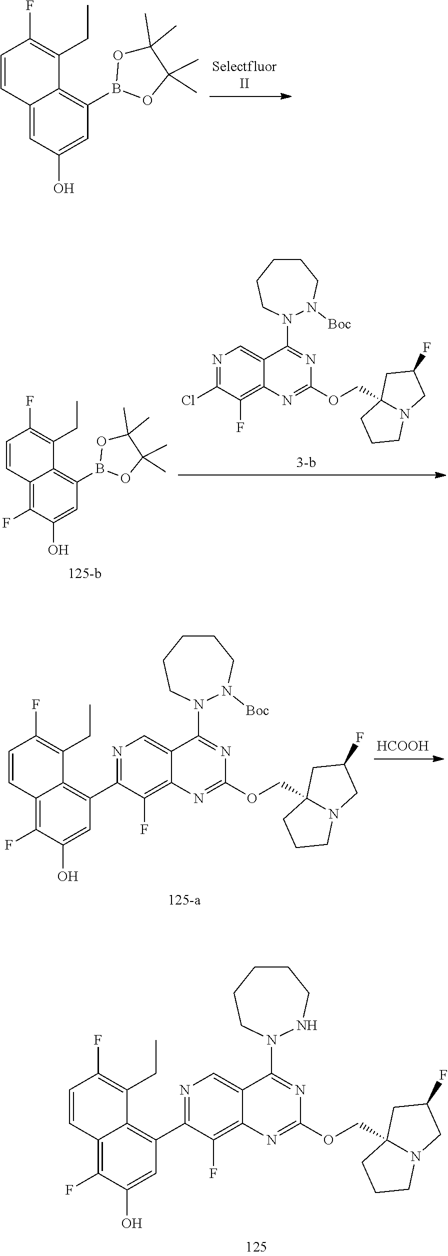

### Synthesis of Compound 125-b

Compound 5-ethyl-6-fluoro-4-(4,4,5,5-tetramethyl-1,3,2-dioxaborolan-2-yl)naphthalen-2-ol (700 mg, 2.21 mmol) was dissolved in acetonitrile (15 mL), followed by addition of selective 1-fluoro-4-methyl-1,4-diazabicyclo[2.2.2]octane tetrafluoroborate (SelectFluor II) (920 mg, 2.88 mmol). The reaction mixture was stirred at room temperature for 12 hours, then was added water (50 mL), extracted with ethyl acetate (50 mL), washed with brine (50 mL), dried over sodium sulfate, filtered to remove the desiccant, and the filtrate was concentrated at reduced pressure to obtain the crude product, which was purified by column chromatography (mobile phase, PE/EA 10/1) to give compound 125-b (250 mg, 34%). LC-MS (ESI): m/z 333.1(M−H)⁻.

### Synthesis of Compound 125-a

[n-butylbis(1-adamantyl)phosphine](2-amino-1,1′-biphenyl-2-yl)palladium(II) methanesulfonate (14 mg, 0.019 mmol) and anhydrous potassium phosphate (59 mg, 0.28 mmol) were added to a solution of compounds 3-b (50 mg, 0.093 mmol) and 125-b (47 mg, 0.14 mmol) in tetrahydrofuran (2 mL) and water (0.5 mL). After blown with nitrogen for 1 minute, the reaction mixture was sealed and stirred at 65° C. for 12 h. Water (50 mL) was added to the reaction mixture and the aqueous phase was extracted with ethyl acetate (50 mL). The organic phase was washed with brine (50 mL) and dried over sodium sulfate. The desiccant was filtered off and the filtrate was concentrated at reduced pressure to obtain the crude product, which was purified by column chromatography (mobile phase, DCM/MeOH 20/1) to obtain compound 125-a (25 mg, 38%). LC-MS (ESI): m/z 711.0(M+H)⁺.

### Synthesis of Compound 125

Formic acid (2 mL) was added to compound 125-a (25 mg, 0.035 mmol), the reaction mixture was stirred at room temperature for 12 h. The reaction mixture was concentrated at reduced pressure to obtain the crude product, which was purified by prep-HPLC (basic conditions) to give compound 125 (7 mg, 33%). LC-MS (ESI): m/z 611.3 (M+H)⁺.

### Synthetic Route of Compound 126

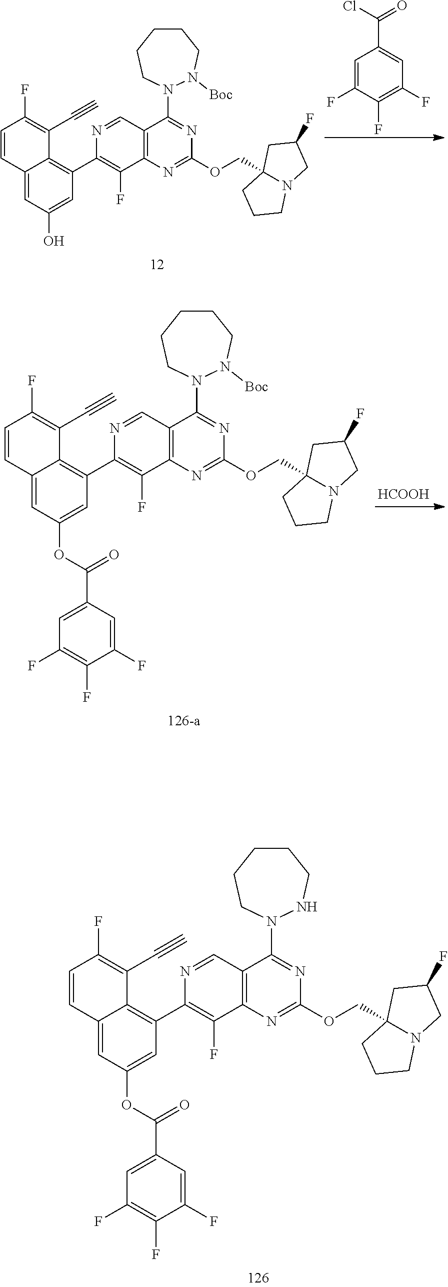

### Synthesis of Compound 126-a

To a solution of compound 12 (100 mg, 0.15 mmol) in dichloromethane (8 mL) were added 3,4,5-trifluorobenzoyl chloride (34 mg, 0.17 mmol) and N,N-diisopropylethylamine (56 mg, 0.44 mmol) at room temperature. The reaction mixture was stirred at this temperature overnight. Water was added to the reaction mixture and the aqueous phase was extracted with dichloromethane (60 mL).

The organic phase was washed with brine, dried over anhydrous sodium sulfate, filtered and concentrated to dryness by rotary evaporation to give 126-a (116 mg, 94%), which was used directly in the next step. LC-MS (ESI): m/z 847.3 (M+H)⁺.

### Synthesis of Compound 126

126-a (116 mg, 0.14 mmol) was dissolved in formic acid (8 mL) and the reaction mixture was stirred at room temperature overnight. The reaction mixture was concentrated to dryness by rotary evaporation, then neutralized by adding saturated aqueous sodium bicarbonate, extracted with ethyl acetate (30 mL*2), the combined organic phases were washed with brine, dried over anhydrous sodium sulfate, filtered, concentrated to dryness by rotary evaporation, and the crude was purified by prep-HPLC to give 126 (16.5 mg, 16%). LC-MS (ESI): m/z 747.3(M+H)⁺.

### Synthetic Route of Compound 127

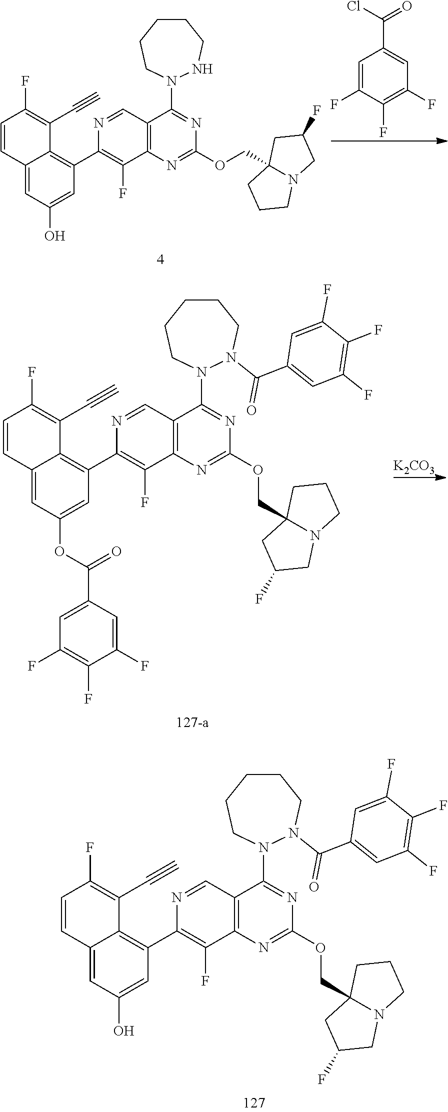

### Synthesis of Compound 127-a

To a solution of compound 4 (100 mg, 0.17 mmol) in dichloromethane (10 mL) were added 3,4,5-trifluorobenzoyl chloride (99 mg, 0.51 mmol) and N,N-diisopropylethylamine (110 mg, 0.85 mmol) at room temperature. The reaction mixture was stirred at this temperature overnight. Water was added and the aqueous phase was extracted with dichloromethane (30 mL), the organic phase was washed with brine, dried over anhydrous sodium sulfate, filtered, and concentrated to dryness by rotary evaporation to give 127-a (145 mg, 94%), which was used directly in the next step. LC-MS (ESI): m/z 905.6 (M+H)⁺.

### Synthesis of Compound 127

127-a (145 mg, 0.16 mmol) was dissolved in methanol (10 mL), potassium carbonate (111 mg, 0.80 mmol) was added and the reaction mixture was stirred for 1 h at room temperature. Concentrated to dryness by rotary evaporation, and the residue was added water, extracted with ethyl acetate (30 mL), the organic phase was washed with brine, dried over anhydrous sodium sulfate, filtered, concentrated to dryness by rotary evaporation, and the crude was purified by prep-HPLC to give 127 (52 mg, 43%). LC-MS (ESI): m/z 747.3(M+H)⁺.

### Synthetic Route of Compound 128

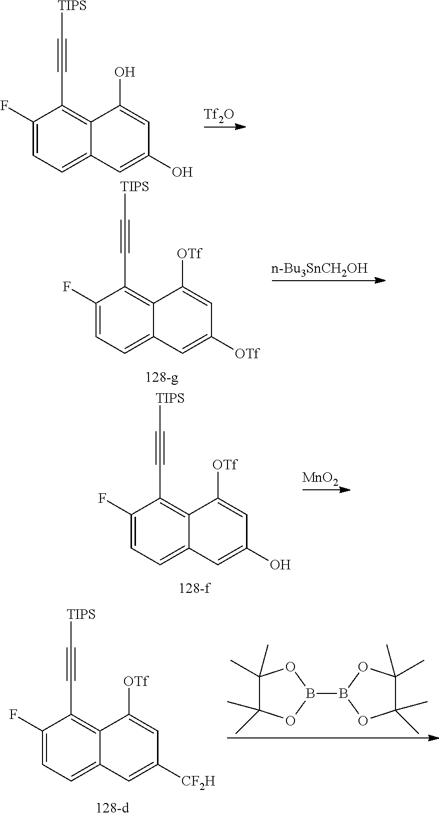

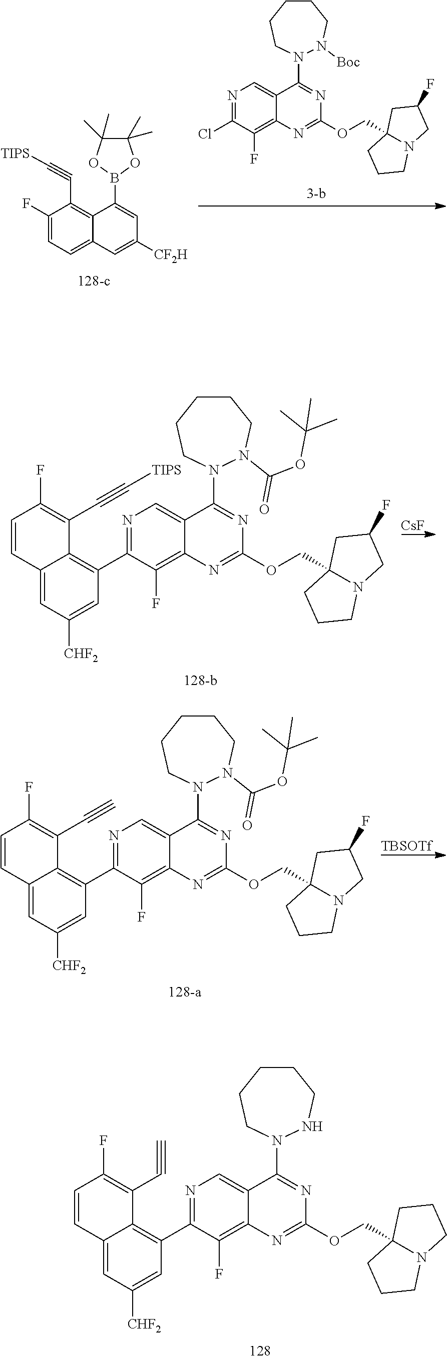

### Synthesis of Compound 128-g

To a reaction flask were added 7-fluoro-8-((triisopropylmethylsilyl)ethynyl)naphthalene-1,3-diol (1560 mg, 4.35 mmol), dichloromethane (80 mL) and DIPEA (3374 mg, 26.11 mmol), respectively. After stirred in an ice-water bath under protection of nitrogen for 10 min, the above mixture was added trifluoromethanesulfonic anhydride (4910 mg, 17.40 mmol) dropwise slowly and stirred for 2 hours in an ice-water bath. Water (100 mL) was added to the reaction mixture and the aqueous phase was extracted with dichloromethane (30 mL*2). The organic phase was concentrated and the residue was purified by column chromatography (EA/PE=0/100, 1/10) to give compound 128-g (2000 mg, 74%). LC-MS (ESI): m/z=640.2(M+NH₄)⁺.

### Synthesis of Compound 128-f

To a microwave tube was added 128-g (780 mg, 1.25 mmol), 1,4-dioxane (2 mL), tributylstannylmethanol (560 mg, 1.74 mmol) and palladium tetrakis(triphenylphosphine) (200 mg, 0.17 mmol). After degassed and purged with nitrogen for three times, the reaction mixture was stirred in an oil bath preheated to 90° C. for 8 hours. The reaction mixture was concentrated and the residue was purified by column chromatography (PE/EA=3/1, 1/3) to afford compound 128-f (156 mg, 25%). LC-MS (ESI): m/z=503.4(M−H)⁻.

### Synthesis of Compound 128-e

To a reaction vial were added 128-f (156 mg, 0.31 mmol), tetrahydrofuran (10 mL) and activated manganese dioxide (269 mg, 3.09 mmol). The reaction mixture was stirred under protection of nitrogen at 40° C. for 2 hours. It was filtered and the organic phase was concentrated to give compound 128-e (150 mg, 97%).

### Synthesis of Compound 128-d

To a reaction flask were added 128-e (120 mg, 0.24 mmol), dichloromethane (4 mL), DAST (1220 mg, 7.57 mmol), and the reaction mixture was stirred under protection of nitrogen for 2 hours at room temperature. It was added water (20 mL), extracted with dichloromethane (20 mL*3). The organic phase was concentrated and the residue was purified by column chromatography (PE/EA=5/1, 3/1) to afford compound 128-d (76 mg, 61%).

### Synthesis of Compound 128-c

To a microwave tube was added 128-d (76 mg, 0.15 mmol), pinacol ester of bisboronic acid (236 mg, 0.93 mmol), potassium acetate (260 mg, 2.65 mmol), PdCl₂(dppf) (35 mg, 0.05 mmol) and dioxyhexacyclohexane (6 mL). After degassed and purged with nitrogen for three times, the reaction mixture was stirred in an oil bath preheated to 100° C. for 1 hour. The reaction mixture was concentrated and the residue was purified by column chromatography (PE/EA=10/1) to give compound 128-c (61 mg, 84%). LC-MS (ESI): m/z=503.1(M−H)⁺.

### Synthesis of Compound 128-b

To a microwave tube were added 128-c (80 mg, 0.16 mmol), tetrahydrofuran (6 mL), water (0.8 mL), 3-b (112 mg, 0.21 mmol), potassium phosphate (135 mg, 0.64 mmol), and cataCXium A Pd G3 (35 mg, 0.05 mmol). After degassed and purged with nitrogen for three times, the reaction mixture was stirred in an oil bath preheated to 65° C. for 12 hours. The reaction mixture was concentrated and the residue was purified by column chromatography (DCM/MeOH=10/1) to afford compound 128-b (72 mg, 51%). LC-MS (ESI): m/z=880.0(M+H)⁺.

### Synthesis of Compound 128-a

To a reaction vial were added 128-b (72 mg, 0.08 mmol), DMF (20 mL) and cesium fluoride (200 mg, 1.32 mmol) at room temperature, and the reaction mixture was stirred for 8 h at room temperature. To the reaction vial was added water (100 mL), and the aqueous phase was extracted with ethyl acetate (100 mL*3). The organic phase was concentrated, and the residue was purified by column chromatography (DCM/MeOH=100/1, 10/1) to give compound 128-a (46 mg, 78%).

LC-MS (ESI): m/z=723.9(M+H)⁺.

### Synthesis of Compound 128

To a reaction flask were added 128-a (56 mg, 0.08 mmol) and dichloromethane (2 mL). After stirred in an ice-water bath for 10 min, it was added TBSOTf (160 μL, 0.09 mmol) slowly and stirred for 1 h in an ice-water bath. The reaction mixture was quenched with saturated aqueous sodium bisulfate (2 mL) and stirred for 1 h at room temperature. It was then neutralized with saturated sodium bicarbonate (6 mL), extracted with dichloromethane (5 mL*2), and the organic phase was concentrated and the residue was purified by prep-HPLC to give compound 128 (11 mg, 23%), LC-MS (ESI): m/z 623.8(M+H)⁺.

### Synthetic Route of Compound 129

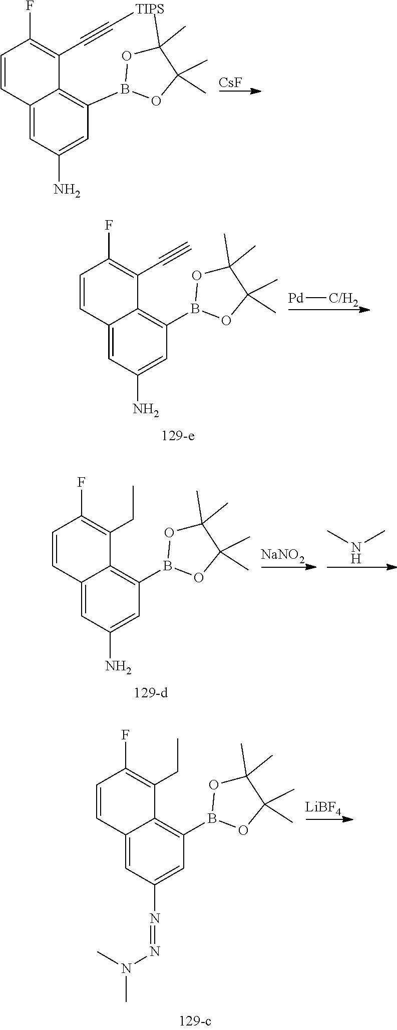

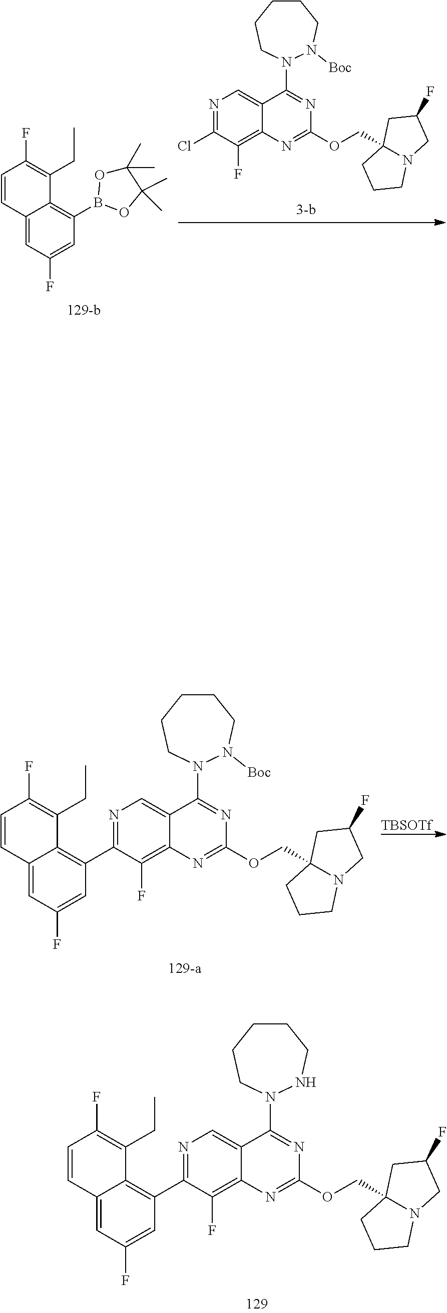

### Synthesis of Compound 129-e

To a reaction vial were added 6-fluoro-4-(4,4,5,5-tetramethyl-1,3,2-dioxaborolan-2-yl)-5-((triisopropylsilyl)ethynyl)naphthalen-2-amine (156 mg, 0.33 mmol), cesium fluoride (507 mg, 3.34 mmol) and DMF (5 mL), and the mixture was stirred at room temperature for 3 hours. The solid was filtered and the filtrate was used in the next step. LC-MS (ESI): m/z=312.1 (M+H)⁺.

### Synthesis of Compound 129-d

129-e (0.33 mmol), palladium carbon (36 mg, 10%) and DMF (5 mL) were added to a reaction vial at room temperature. After degassed and purged with hydrogen (1000 mL) for three times, the reaction mixture was stirred for 2 h at this temperature. The reaction mixture was concentrated at room temperature. Water (30 mL) was added to the residue and the aqueous phase was extracted with ethyl acetate (30 mL*3). The organic phase was concentrated and the residue was purified by column chromatography (PE/EA=5/1, 3/1) to give compound 129-d (67 mg, 64%). LC-MS (ESI): m/z=316.2(M+H)⁺.

### Synthesis of Compound 129-c

129-d (56 mg, 0.18 mmol), acetonitrile (5 mL), concentrated hydrochloric acid (26 mg, 0.71 mmol), and water (3 mL) were added to a reaction flask in an ice-salt bath, followed by stirring under protection of nitrogen for 10 min. Sodium nitrite (49 mg, 0.71 mmol) was dissolved water (1 mL), followed by slow addition to the above mixture in an ice-salt bath. After addition, the reaction mixture continued stirring in an ice-water bath for 45 minutes. Potassium carbonate (245 mg, 1.78 mmol) and dimethylamine (0.24 mL, 3.55 mmol) were added to water (2 mL) and then this solution was added dropwise to the reaction mixture. After addition, the reaction mixture continued stirring in an ice-water bath for 1 hour. It was extracted with ethyl acetate (90 mL*3), dried over sodium sulfate, filtered, the organic phase was concentrated and the residue was purified by column chromatography (PE/EA=3/1) to give compound 129-c (46 mg, 70%). LC-MS (ESI): m/z=372.2(M+H)⁺.

### Synthesis of Compound 129-b

129-c (41 mg, 0.11 mmol), hexafluorobenzene (2 mL), ethyl acetate (2 mL) and lithium tetrafluoroborate (52 mg, 0.55 mmol) were added to a microwave tube at room temperature. After degassed and purged with nitrogen for three times, the microwave tube was placed into an oil bath preheated to 90° C. and stirred for 2 hours. The reaction mixture was concentrated to dryness by rotary evaporation and the residue was purified by column chromatography (PE/EA=3/1) to give compound 129-b (23 mg, 65%).

### Synthesis of Compound 129-a

To a microwave tube were added 129-b (20 mg, 0.06 mmol), tetrahydrofuran (6 mL), water (0.8 mL), 3-b (45 mg, 0.08 mmol), potassium phosphate (54 mg, 0.26 mmol) and cataCXium A Pd G3 (14 mg, 0.02 mmol). After degassed and purged with nitrogen for three times, the microwave tube was placed into an oil bath preheated to 65° C. and stirred for 12 hours. The reaction mixture was concentrated and the residue was purified by column chromatography (DCM/MeOH=10/1) to afford compound 129-a (16 mg, 37%). LC-MS (ESI): m/z=695.9(M+H)⁺.

### Synthesis of Compound 129

To a reaction flask were added 129-a (16 mg, 0.02 mmol) and dichloromethane (2 mL) at room temperature. The mixture was stirred in an ice-water bath for 10 min, was added TBSOTf (69 mg, 0.26 mmol) slowly and stirred in an ice-water bath for 1 hr. The reaction mixture was added saturated aqueous sodium bicarbonate solution (10 mL) and stirred at room temperature for 6 h. The reaction mixture was extracted with dichloromethane (10 mL*3), the organic phase was concentrated and the residue was purified by prep-HPLC to give compound 129 (5 mg, 36%). LC-MS (ESI): m/z 595.3(M+H)⁺.

### Synthetic Route of Compound 130

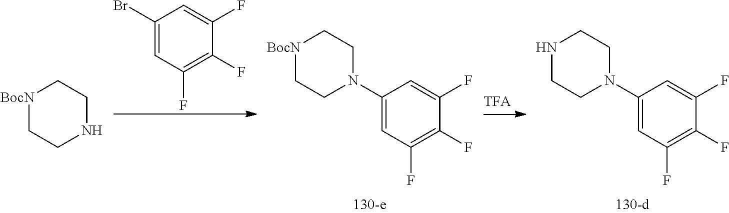 [SMILES: CC(C)(C)OC(=O)N1CCNCC1 ; Fc1cc(Br)cc(F)c1F ; CC(C)(C)OC(=O)N1CCN(c2cc(F)c(F)c(F)c2)CC1 ; Fc1cc(N2CCNCC2)cc(F)c1F]

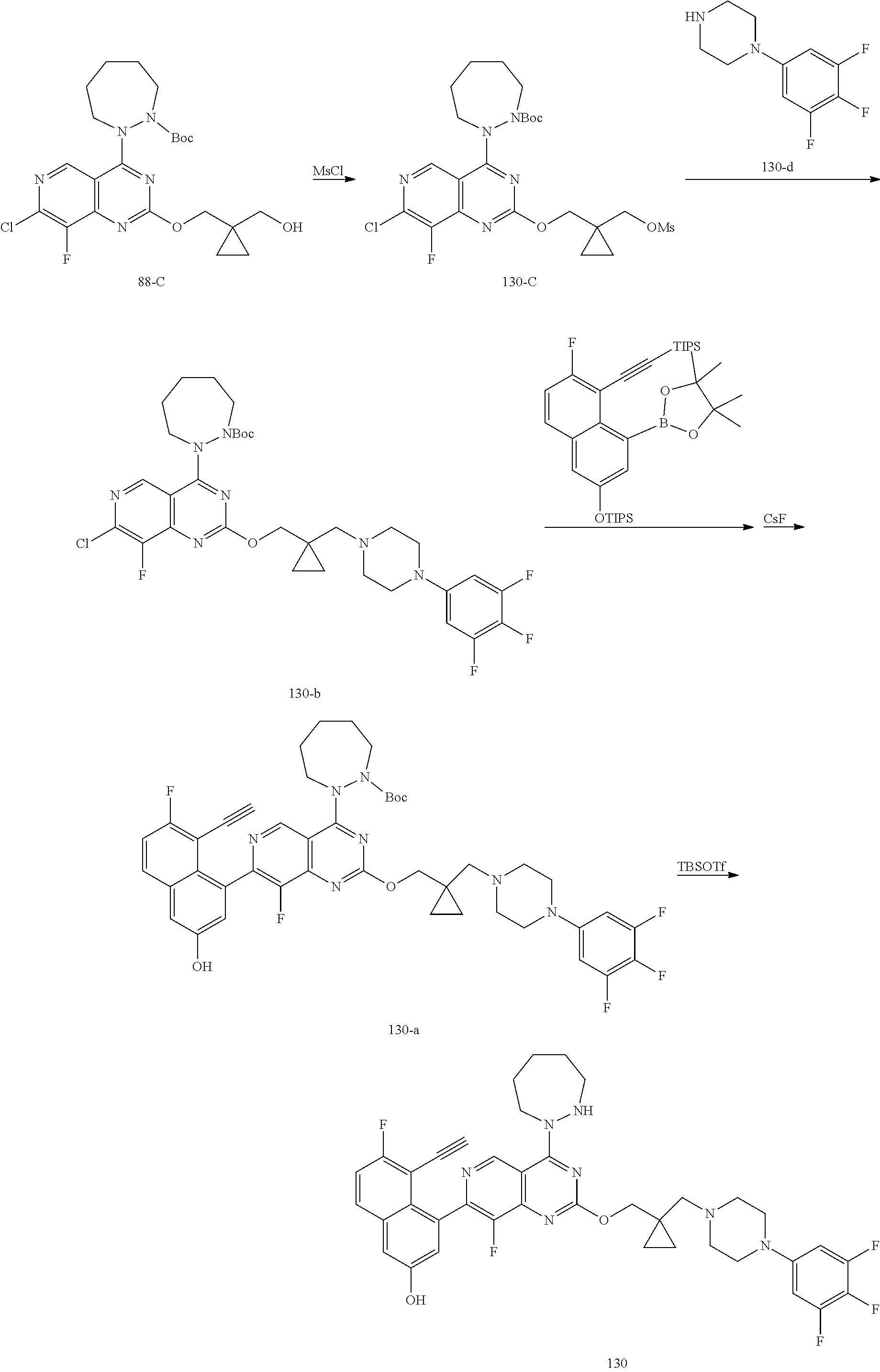

### Synthesis of Compound 130-e

To a reaction flask were added 3,4,5-trifluorobromobenzene (3 g, 14.22 mmol), N-Boc-piperazine (3.71 g, 19.92 mmol), tris(dibenzylideneacetone)dipalladium (1.3 g, 1.42 mmol), 1,3-bis(2,6-diisopropylphenyl)imidazolium chloride (1.21 g, 2.84 mmol), sodium tert-butoxide (2.05 g, 21.33 mmol) and toluene (30 mL), the mixture was stirred at 100° C. for 2 hours. The reaction mixture was cooled to room temperature, water (20 mL) was added and the aqueous phase was extracted with ethyl acetate (50 mL). The organic phase was concentrated and the residue was purified by column chromatography (mobile phase: ethyl acetate: petroleum ether=0-20%) to give 130-e (3.00 g, 67%).

LCMS (ESI): m/z 317.1 (M+H)⁺.

### Synthesis of Compound 130-d

130-e (4.2 g, 13.28 mmol) and dichloromethane (30 mL) were added to a reaction flask in an ice-water bath and trifluoroacetic acid (15.14 g, 133 mmol) was added dropwise. After addition, the reaction mixture was stirred in an ice-water bath for 2 hours, then was concentrated. The residue was dissolved with dichloromethane (30 mL), the pH was adjusted to 9-10 with saturated aqueous sodium carbonate, the phases were separated, and the organic phase was dried over anhydrous anhydrous sodium sulfate, filtered, and concentrated to give 130-d (2.40 g, 84%). LCMS (ESI): m/z 217.1 (M+H)⁺.

### Synthesis of Compound 130-c

To a reaction vial in an ice-water bath was added 88-c (800 mg, 1.66 mmol), N,N-diisopropylethylamine (644 mg, 4.98 mmol) and dichloromethane (20 mL), and methanesulfonyl chloride (570 mg, 4.98 mmol) was added dropwise. After addition, the reaction mixture was stirred in an ice-water bath for 30 minutes. Water (30 mL) was added to the reaction mixture and the aqueous phase was extracted with dichloromethane (30 mL), the phases were separated and the aqueous phase was extracted with dichloromethane (15 mL). The organic phases were combined, dried over anhydrous sodium sulfate, filtered, and the filtrate was concentrated to dryness by rotary evaporation and the residue was purified by column chromatography (mobile phase: ethyl acetate: petroleum ether=0-80%) to give 130-c (870 mg, 94%). LCMS (ESI): m/z 560.4 (M+H)⁺.

### Synthesis of Compound 130-b

To a reaction vial were added 130-c (870 mg, 1.55 mmol), 130-d (336 mg, 1.55 mmol), N,N-diisopropylethylamine (602 mg, 4.66 mmol), potassium iodide (258 mg, 1.55 mmol) and N,N-dimethylformamide (10 mL). The mixture was stirred at 90° C. for 2 h under protection of nitrogen, then was cooled to room temperature, was added water (30 mL) and the aqueous phase was extracted with ethyl acetate (50 mL). The organic phase was concentrated and the residue was purified by column chromatography (mobile phase: ethyl acetate: petroleum ether=0-80%) to give 130-b (500 mg, 47%).

### Synthesis of Compound 130-a

To a reaction vial were added 130-b (500 mg, 0.74 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (459 mg, 0.74 mmol), methanesulfonic acid [n-butylbis(1-adamantyl)phosphine](2-amino-1,1′-biphenyl-2-yl)palladium(II) (54 mg, 0.074 mmol), potassium phosphate (468 mg, 2.21 mmol), water (2 mL) and tetrahydrofuran (10 mL). After degassed and purged with nitrogen for three times, the reaction mixture was heated at 65° C. for 3 hours. The reaction mixture was cooled to room temperature, was added water (30 mL) and extracted twice with ethyl acetate (20 mL). The organic phase was dried over anhydrous sodium sulfate, filtered, and the filtrate was concentrated. The crude product obtained was directly dissolved with N,N-dimethylformamide (10 mL), followed by addition of cesium fluoride (1.00 g, 6.58 mmol) and the mixture was stirred at room temperature for 2 hours. It was added water (20 mL) and extracted twice with ethyl acetate (20 mL). The organic phase was concentrated and the residue was purified by column chromatography (mobile phase: ethyl acetate: petroleum ether=0-40%) to give 130-a (340 mg, 56%). LCMS (ESI): m/z 830.9 (M+H)⁺.

### Synthesis of Compound 130

Tert-butyldimethylsilyl trifluoromethanesulfonate (1.43 g, 5.42 mmol) was added dropwise to a solution of 130-a (300 mg, 0.36 mmol) in dichloromethane (8 mL) in a reaction flask in an ice-water bath. After addition, the reaction mixture was stirred in an ice-water bath for half an hour, then was added saturated aqueous sodium bicarbonate (20 mL) and stirred at room temperature overnight. The organic phase was dried and concentrated and the residue was purified by column chromatography (mobile phase: ethyl acetate: petroleum ether=0-50%) to give 130 (40 mg, 15%).

LCMS(ESI): m/z 730.4 (M+H)⁺; ¹H NMR (400 MHz, CDCl₃): δ 10.18-9.90 (m, 2H), 7.50 (s, 1H), 7.21 (s, 1H), 7.16-6.92 (m, 2H), 6.51-6.25 (m, 2H), 4.57-3.94 (m, 5H), 3.24-2.94 (m, 6H), 2.81-2.27 (m, 7H), 2.00-1.88 (m, 2H), 1.78-1.66 (m, 4H), 0.75-0.64 (m, 2H), 0.56-0.34 (m, 2H).

### Synthetic Route of Compound 131

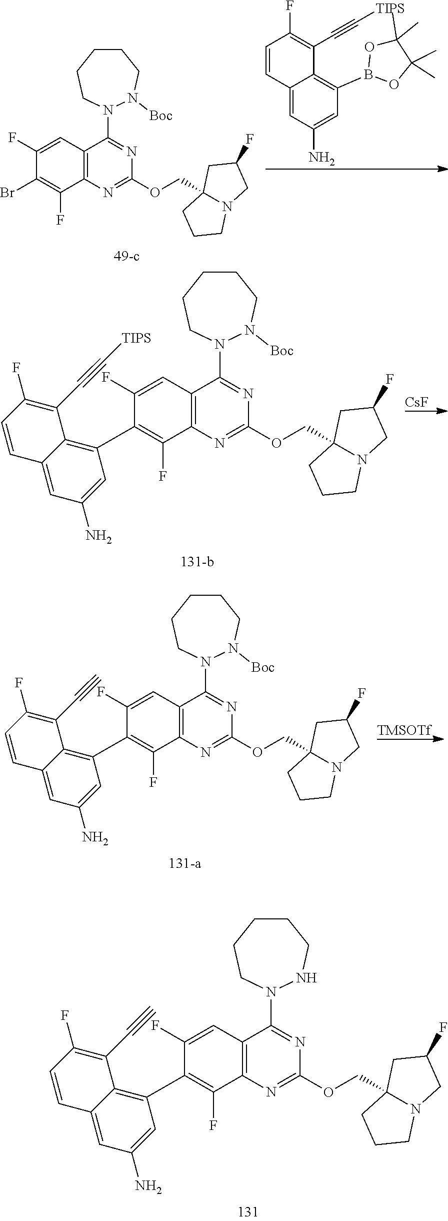

### Synthesis of Compound 131-b

To a microwave tube was added 49-c (50 mg, 0.083 mmol), 6-fluoro-4-(4,4,5,5-tetramethyl-1,3,2-dioxaborolan-2-yl)-5-((triisopropylsilyl)ethynyl)naphthalen-2-amine (59 mg, 0.13 mmol), palladium tetrakis(triphenylphosphine) (10 mg, 0.008 mmol), sodium carbonate (27 mg, 0.25 mmol), 1,4-dioxane (3 mL) and water (0.5 mL). After sealed, degassed and purged with nitrogen for three times, the reaction mixture was heated to 100° C. and stirred overnight. The reaction mixture was concentrated at reduced pressure and the residue was purified by column chromatography (mobile phase: methanol/dichloromethane, 0-10%) to give 131-b (40 mg, 56%).

LC-MS (ESI): m/z 861.9 (M+H)⁺.

### Synthesis of Compound 131-a

To a solution of 131-b (40 mg, 0.046 mmol) in N,N-dimethylformamide (4 mL) was added cesium fluoride (70 mg, 0.46 mmol) at room temperature, and the mixture was stirred for 1 h at room temperature. The reaction mixture was poured a large amount of water, extracted with ethyl acetate (20 mL*2), the organic phase was washed with brine (15 mL*3), concentrated to dryness by rotary evaporation, and the residue was purified by column chromatography (mobile phase: methanol/dichloromethane, 0-10%) to give 131-a (20 mg, 61%). LC-MS (ESI): m/z 705.7 (M+H)⁺.

### Synthesis of Compound 131

To a solution of 131-a (20 mg, 0.028 mmol) in dichloromethane (4 mL) was added trimethylsilyl trifluoromethanesulfonate (0.079 mL, 0.34 mmol) at room temperature, and the mixture was stirred for 1 h at room temperature. The reaction was quenched by the addition of saturated sodium bicarbonate (10 mL) solution and the mixture was stirred at room temperature overnight. After completion, the mixture was extracted with dichloromethane (15 mL*2), the organic phase was washed with brine (10 mL*3), dried over anhydrous sodium sulfate, filtered, and concentrated to dryness by rotary evaporation. The crude product was purified by Prep-HPLC (basic method) and lyophilized to give compound 131 (10 mg, 58%). LC-MS (ESI): m/z 605.3 (M+H)⁺.

### Synthetic Route of Compound 132

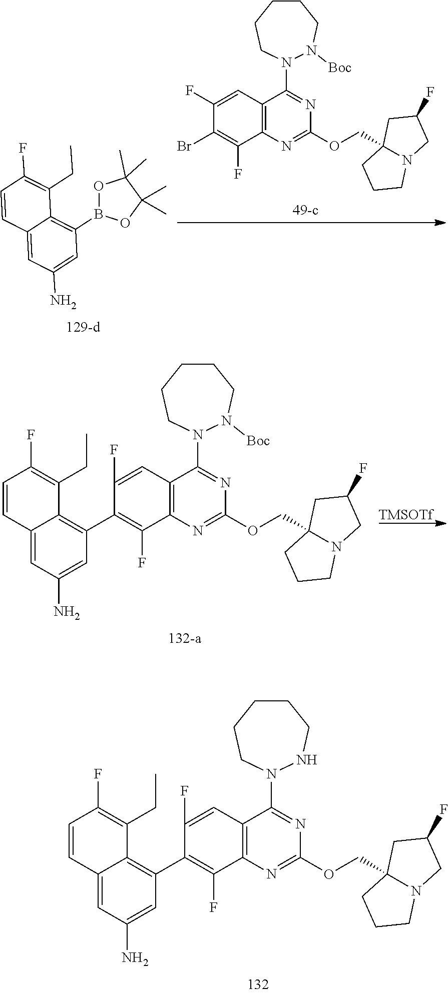 [SMILES: CCc1c(F)ccc2cc(N)cc(B3OC(C)(C)C(C)(C)O3)c12 ; "TMSOTf" ; CC(C)(C)OC(=O)N1CCCCCN1c1nc(OC[C@@]23CCCN2C[C@H](F)C3)nc2c(F)c(Br)c(F)cc12 ; CCc1c(F)ccc2cc(N)cc(-c3c(F)cc4c(N5CCCCCN5C(=O)OC(C)(C)C)nc(OC[C@@]56CCCN5C[C@H](F)C6)nc4c3F)c12 ; CCc1c(F)ccc2cc(N)cc(-c3c(F)cc4c(N5CCCCCN5)nc(OC[C@@]56CCCN5C[C@H](F)C6)nc4c3F)c12]

### Synthesis of Compound 132-a

49-c (50 mg, 0.083 mmol), 129-d (40 mg, 0.13 mmol), palladium tetrakis(triphenylphosphine) (10 mg, 0.008 mmol), sodium carbonate (26 mg, 0.25 mmol), 1,4-dioxane (3 mL), and water (0.5 mL) were added to a microwave tube. After sealed, degassed and purged with nitrogen for three times, the reaction mixture was heated to 100° C. and stirred overnight. On the next day, the reaction mixture was cooled to room temperature, concentrated at reduced pressure, and the resulting crude product was purified by column chromatography (mobile phase: methanol/dichloromethane, 0-10%) to give 132-a (40 mg, 68%). LC-MS (ESI): m/z 709.7 (M+H)⁺.

### Synthesis of Compound 132

To a solution of 132-a (40 mg, 0.056 mmol) in dichloromethane (4 mL) was added trimethylsilyl trifluoromethanesulfonate (0.077 mL, 0.33 mmol) at room temperature, and the mixture was stirred for 1 h at room temperature. The reaction was quenched by addition of saturated sodium bicarbonate solution (10 mL) and the mixture was stirred at room temperature overnight. Upon completion, the reaction mixture was extracted with dichloromethane (15 mL*2), the organic phase was washed with brine (10 mL*3), dried over anhydrous sodium sulfate, filtered, and concentrated to dryness by rotary evaporation. The crude product was purified by Prep-HPLC (basic method) and lyophilized to give compound 132 (25 mg, 73%). LC-MS (ESI): m/z 609.5 (M+H)⁺.

### Synthesis of Compound 133

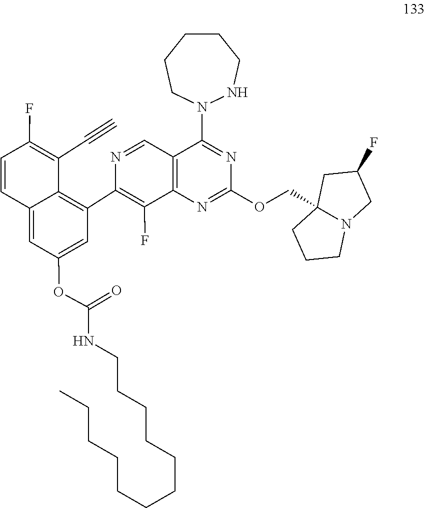 [SMILES: C#Cc1c(F)ccc2cc(OC(=O)NCCCCCCCCCCCC)cc(-c3ncc4c(N5CCCCCN5)nc(OC[C@@]56CCCN5C[C@H](F)C6)nc4c3F)c12]

Referring to the synthetic route of compound 69, compound 133 was obtained using dodecyl isocyanate instead of n-octyl isocyanate. LC-MS (ESI): m/z 800.5 [M+H]⁺; ¹H NMR (400 MHz, CDCl₃): δ 10.25 (1H, s), 7.86 (1H, dd, J=8.8, 5.6 Hz), 7.79 (1H, d, J=2.4 Hz), 7.44 (1H, d, J=2.4 Hz), 7.31 (1H, t, J=8.8 Hz), 5.30 (1H, d, J=53.6 Hz), 5.13-5.22 (1H, m), 3.97-4.37 (5H, m), 3.08-3.53 (7H, m), 2.94-3.08 (1H, m), 2.86 (1H, s), 2.12-2.44 (3H, m), 1.86-2.09 (5H, m), 1.66-1.85 (4H, m), 1.50-1.66 (2H, m), 1.17-1.44 (18H, m), 0.84-0.92 (3H, m).

### Synthetic Route of Compound 134

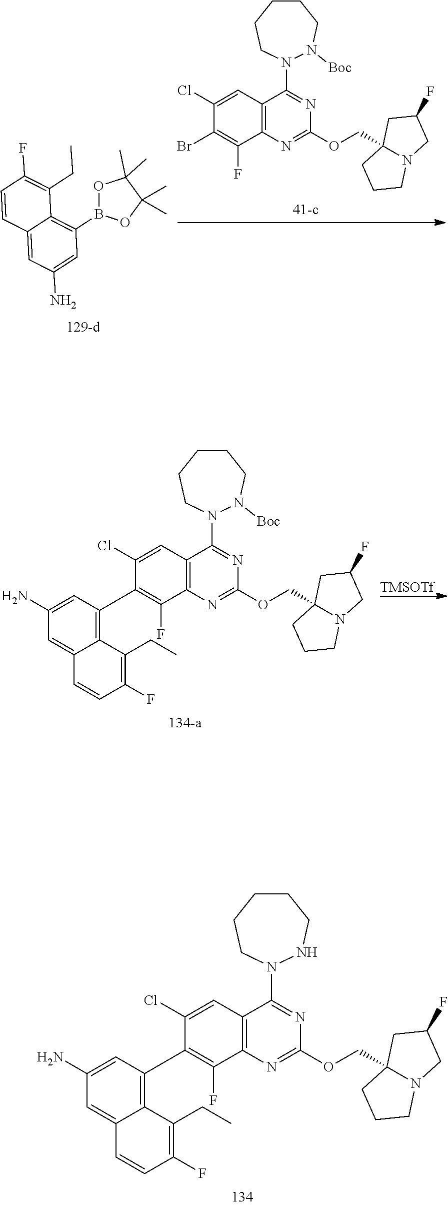

### Synthesis of Compound 134-a

41-c (170 mg, 0.28 mmol), 129-d (130 mg, 0.41 mmol), palladium tetrakis(triphenylphosphine) (32 mg, 0.028 mmol), sodium carbonate (88 mg, 0.83 mmol), 1,4-dioxane (9 mL) and water (1 mL), were added to a microwave tube. After sealed, degassed and purged with nitrogen for three times, the reaction mixture was heated to 100° C. and stirred overnight. On the next day, the reaction mixture was cooled to room temperature, concentrated at reduced pressure, and the crude product was purified by column chromatography (mobile phase: methanol/dichloromethane, 0-10%) to give 134-a (120 mg, 60%). LC-MS (ESI): m/z 725.7 (M+H)⁺.

### Synthesis of Compound 134

To a solution of 134-a (30 mg, 0.041 mmol) in dichloromethane (4 mL) was added trimethylsilyl trifluoromethanesulfonate (0.057 mL, 0.25 mmol) at room temperature, and the mixture was stirred for 1 h at room temperature. The reaction was quenched by the addition of saturated sodium bicarbonate solution (10 mL) and the mixture was stirred at room temperature overnight. At the end of the reaction, the mixture was extracted with dichloromethane (15 mL*2), the organic phase was washed with brine (10 mL*3), dried over anhydrous sodium sulfate, filtered, and concentrated to dryness by rotary evaporation. The crude was purified by Prep-HPLC (basic method) and lyophilized to give compound 134 (5 mg, 19%). LC-MS (ESI): m/z 625.3 (M+H)⁺.

### Synthetic Route of Compound 135

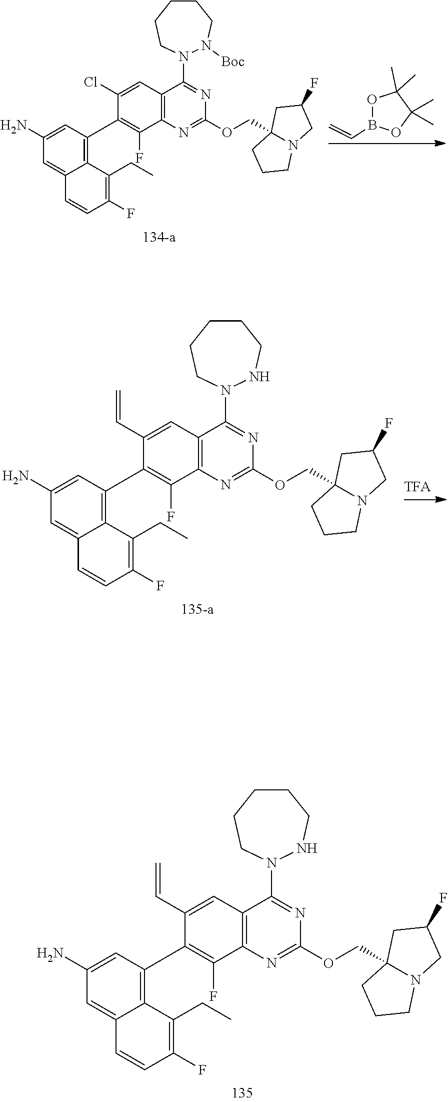

### Synthesis of Compound 135-a

To a microwave tube were added 134-a (90 mg, 0.12 mmol), methanesulfonic acid (2-dicyclohexylphosphino-2′,6′-dimethoxy-1,1′-biphenyl)(2′-amino-1,1′-biphenyl-3-yl)palladium(II) (10 mg, 0.012 mmol), potassium phosphate (79 mg, 0.37 mmol), dioxane (12 mL) and water (2 mL). After sealed, degassed and purged with nitrogen for three times, the above mixture was added vinylboronic acid pinacol ester (38 mg, 0.25 mmol) by syringe, microwaved at 150° C. for 40 min. At the end of the reaction, the reaction mixture was cooled to room temperature, concentrated at reduced pressure, and the crude product was purified by column chromatography (mobile phase: methanol/dichloromethane, 0-10%) to obtain 135-a (70 mg, 79%). LC-MS (ESI): m/z 717.5 (M+H)⁺. Synthesis of compound 135 Trifluoroacetic acid (3 mL) was added to a solution of 135-a (70 mg, 0.098 mmol) in dichloromethane (10 mL) at room temperature, and the mixture was stirred for 1 hour at room temperature. The reaction mixture was concentrated to dryness by rotary evaporation, and the residue was dissolved in methanol, neutralized to pH 8 with saturated sodium bicarbonate solution, concentrated to dryness by rotary evaporation, and the residue was purified by column chromatography (mobile phase: ammonia methanol: dichloromethane=0-10%). The resulting crude was purified by Prep-HPLC (basic method) to give 135 (20 mg, 33%). LC-MS (ESI): m/z 617.3 (M+H)⁺.

### Synthetic Route of Compound 136

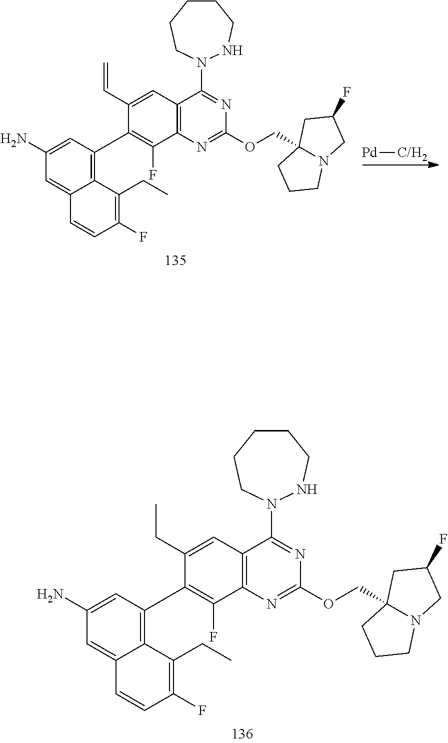

### Synthesis of Compound 136

To a solution of 135 (15 mg, 0.024 mmol) in ethanol (5 mL) was added 10% palladium carbon (3 mg, 0.002 mmol) at room temperature, degassed and purged with hydrogen for three times, and the mixture was stirred for 4 hours at room temperature. The reaction mixture was filtered and the filtrate was concentrated to dryness by rotary evaporation, purified by prep-HPLC (ammonium bicarbonate) and lyophilized to give 136 (2 mg, 13%). LC-MS (ESI): m/z 619.3 (M+H)⁺.

### Synthetic Route of Compound 137

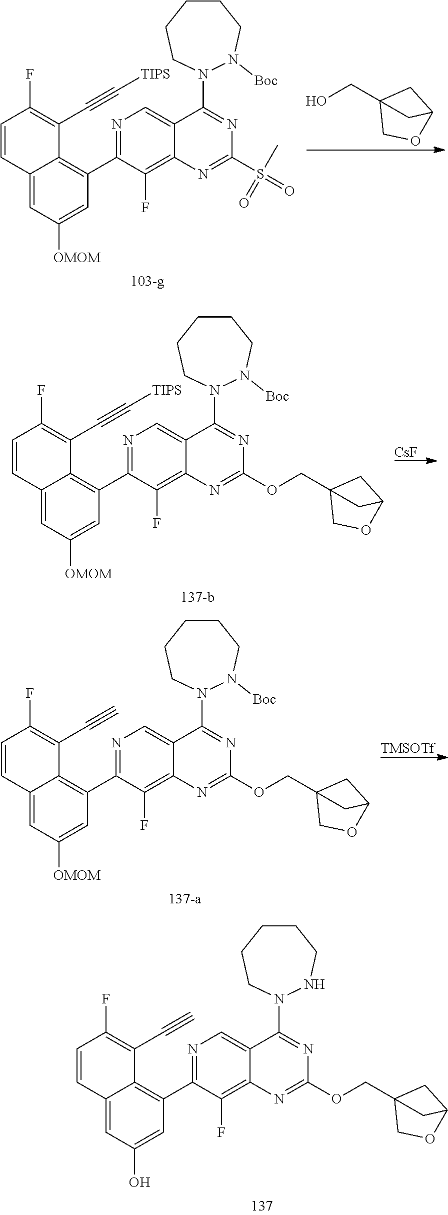

### Synthesis of Compound 137-b

To a reaction vial at room temperature was added 103-g (150 mg, 0.19 mmol), toluene (5 mL), 2-oxabicyclo[2.1.1]hexan-4-ylmethanol (42 mg, 0.37 mmol). After degassed and purged with nitrogen, cooled down to 0° C., the mixture was added sodium tert-butoxide (53 mg, 0.55 mmol) and stirred at 0° C. for 1 hour. The reaction mixture was mixed into silica gel and purified by column chromatography (mobile phase: PE/EA 10/0 to 5/5) to give compound 137-b (150 mg, 96%). LC-MS (ESI): m/z844.5 (M+H)⁺.

### Synthesis of Compound 137-a

To a reaction vial at room temperature were added 137-b (150 mg, 0.18 mmol), DMF (2 mL) and cesium fluoride (270 mg, 1.78 mmol). After degassed and purged with nitrogen, the mixture was heated at 50° C. for 1 h. The reaction mixture was added water and the aqueous phase was extracted with EA. The organic phases were combined, washed with water, brine, dried over anhydrous sodium sulfate, and concentrated to give the crude compound 137-a (170 mg). LC-MS (ESI): m/z 688.7 (M+H)⁺.

### Synthesis of Compound 137

To a reaction vial at room temperature was added crude 137-a (170 mg, 0.18 mmol) and DCM (3 mL). After degassed and purged with nitrogen, and cooled down to 0° C., the mixture was added trimethylsilyl trifluoromethanesulfonate (0.3 mL) dropwise, then stirred for 2 hours. 10% aqueous sodium bisulfate (3 mL) was added and stirred for 1 hour. The reaction mixture was extracted with DCM. The organic phases were combined, washed with saturated aqueous sodium bicarbonate, washed with water, brine, dried, concentrated, and the residue was purified by prep-HPLC to give compound 137 (9 mg, 9%). LC-MS (ESI): m/z 544.3 (M+H)⁺.

### Synthetic Route of Compound 138

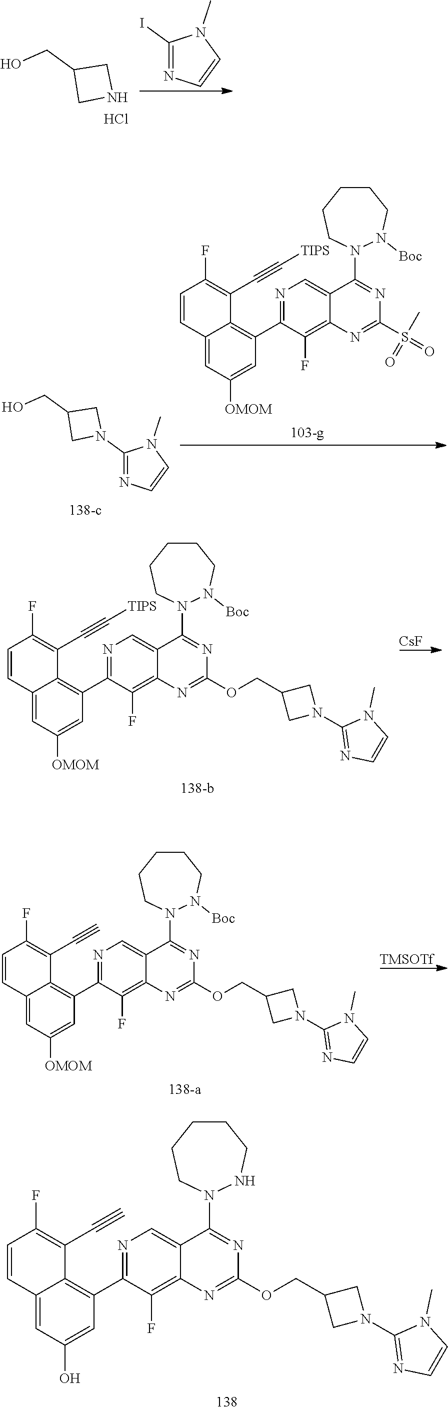

### Synthesis of Compound 138-c

To a reaction vial at room temperature were added 3-azetidinemethanol hydrochloride (500 mg, 4.05 mmol), DMF (10 mL), cuprous iodide (77 mg, 0.40 mmol), cesium carbonate (2637 mg, 8.09 mmol), D-proline (93 mg, 0.81 mmol), 2-iodo-1-methyl-1H-imidazole (1010 mg, 4.86 mmol). The mixture was degassed and purged with nitrogen, heated to 100° C. and stirred for 5 hours. The reaction mixture was concentrated, purified by column chromatography (mobile phase: DCM/MeOH 10/0 to 10/1) to give compound 138-c (100 mg, 15%). LC-MS (ESI): m/z 168.2 (M+H)⁺.

### Synthesis of compound 138-b

103-g (200 mg, 0.25 mmol), toluene (7 mL) and 138-c (100 mg, 0.60 mmol) were added to a reaction vial at room temperature. After degassed and purged with nitrogen, cooled down to 0° C., the mixture was added sodium tert-butoxide (71 mg, 0.74 mmol), and stirred at 0° C. for 1 hour. The reaction mixture was mixed into silica gel and purified by column chromatography (mobile phase: PE/EA 10/0 to 4/6) to give compound 138-b (54 mg, 24%). LC-MS (ESI): m/z 897.6 (M+H)⁺.

### Synthesis of compound 138-a

138-b (54 mg, 0.06 mmol), DMF (2 mL) and cesium fluoride (91 mg, 0.60 mmol) were added to a reaction flask at room temperature. After degassed and purged with nitrogen, the mixture was heated at 50° C. for 1 h. The reaction mixture was added water and the aqueous phase was extracted with EA. The organic phases were combined, washed with water, brine, dried over anhydrous sodium sulfate, and concentrated to give the crude compound 138-a (80 mg). LC-MS (ESI): m/z 741.8 (M+H)⁺.

### Synthesis of Compound 138

To a reaction vial at room temperature were added crude 138-a (80 mg, 0.06 mmol) and DCM (3 mL). After degassed and purged with nitrogen and cooled down to 0° C., the mixture was added trimethylsilyl trifluoromethanesulfonate (0.3 mL) dropwise, then stirred for 2 hours. 10% aqueous sodium bisulfate (3 mL) was added and stirred for 1 hour. The reaction mixture was extracted with DCM. The organic phases were combined, washed with saturated aqueous sodium bicarbonate, washed with water, brine, dried, concentrated and the residue was purified by prep-HPLC to give compound 138 (10 mg, 28%). LC-MS (ESI): m/z 597.3 (M+H)⁺.

### Synthetic Route of Compound 139

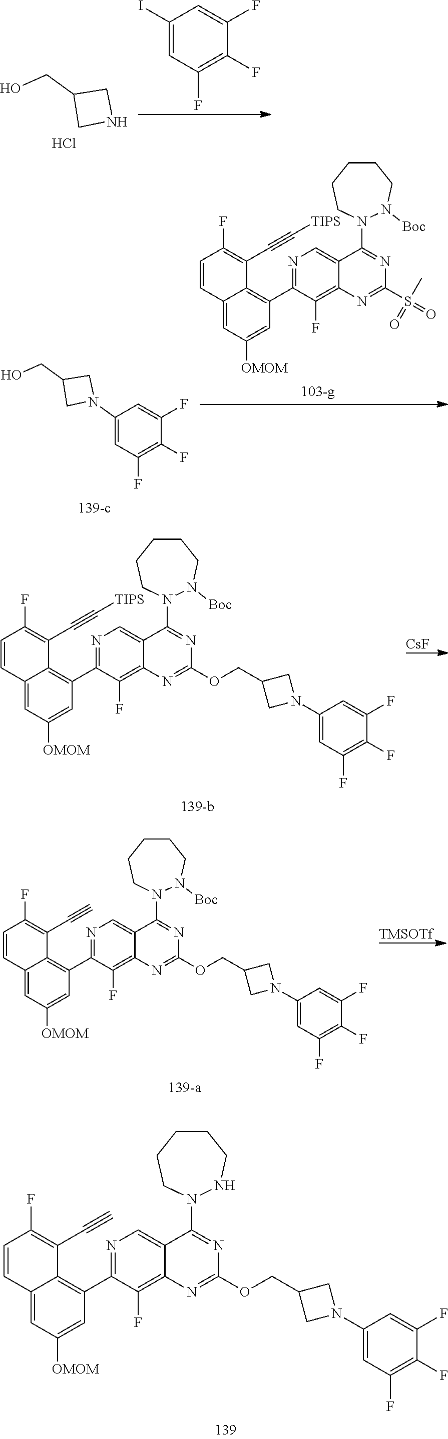

### Synthesis of Compound 139-c

To a reaction vial at room temperature was added 3-azetidinemethanol hydrochloride (500 mg, 4.05 mmol), DMF (10 mL), cuprous iodide (77 mg, 0.40 mmol), cesium carbonate (2637 mg, 8.09 mmol), D-proline (93 mg, 0.81 mmol), and 3,4,5-trifluoroiodobenzene (1253 mg, 4.86 mmol). After degassed and purged with nitrogen, the mixture was heated to 100° C. and stirred for 5 hours. The reaction mixture was concentrated, purified by column chromatography (mobile phase: DCM/MeOH 10/0 to 10/1) to give compound 139-c (700 mg, 43%). LC-MS (ESI): m/z 218.1 (M+H)⁺.

### Synthesis of Compound 139-b

103-g (200 mg, 0.25 mmol), toluene (5 mL), and 139-c (100 mg, 0.46 mmol) were added to a reaction vial at room temperature. After degassed and purged with nitrogen, cooled down to ° C., the mixture was added sodium tert-butoxide (95 mg, 1.00 mmol), and stirred at 0° C. for 1 hour. The reaction mixture was mixed into silica gel anied by column chromatography (mobile phase: PE/EA 10/0 to 6/4) to give compound 139-b (200 mg, 86%).

### Synthesis of Compound 139-a

To a reaction vial at room temperature was added 139-b (200 mg, 0.21 mmol), DMF (3 mL), and cesium fluoride (321 mg, 2.11 mmol). After degassed and purged with nitrogen, the mixture was heated to 50° C. for 1 h. The reaction mixture was added water and the aqueous phase was extracted with EA. The organic phases were combined, washed with water, brine, dried over anhydrous sodium sulfate, and concentrated to give the crude compound 139-a (200 mg). LC-MS (ESI): m/z 791.5 (M+H)⁺.

### Synthesis of Compound 139

To a reaction vial at room temperature were added crude 139-a (200 mg, 0.21 mmol), DCM (5 mL). After degassed and purged with nitrogen and cooled down to 0° C., the mixture was added trimethylsilyl trifluoromethanesulfonate (0.5 mL) dropwise, then stirred for 2 hours. 10% aqueous sodium bisulfate (3 mL) was added and stirred for 1 hour. The reaction mixture was extracted with DCM. The organic phases were combined, washed with saturated aqueous sodium bicarbonate, washed with water, brine, dried, concentrated and the residue was purified by prep-HPLC to give compound 139 (10 mg, 28%). LC-MS (ESI): m/z 647.6 (M+H)⁺.

### Synthetic Route of Compound 140

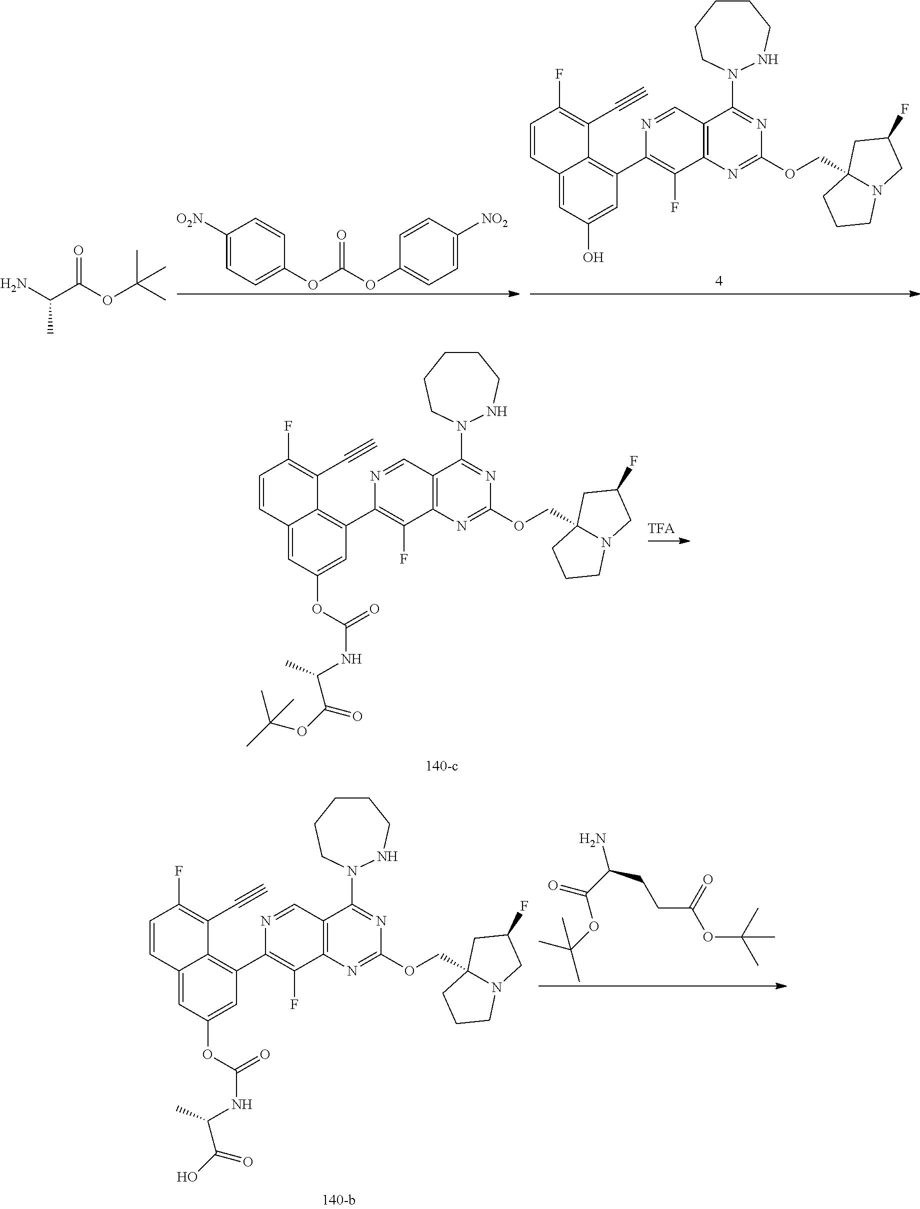

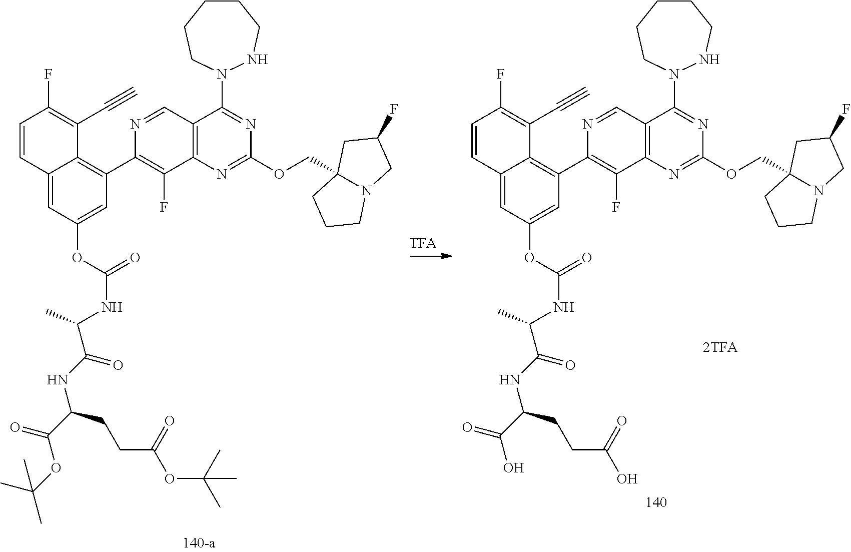 [SMILES: C#Cc1c(F)ccc2cc(OC(=O)N[C@@H](C)C(=O)N[C@@H](CCC(=O)OC(C)(C)C)C(=O)OC(C)(C)C)cc(-c3ncc4c(N5CCCCCN5)nc(OC[C@@]56CCCN5C[C@H](F)C6)nc4c3F)c12 ; C#Cc1c(F)ccc2cc(OC(=O)N[C@@H](C)C(=O)N[C@@H](CCC(=O)O)C(=O)O)cc(-c3ncc4c(N5CCCCCN5)nc(OC[C@@]56CCCN5C[C@H](F)C6)nc4c3F)c12]

### Synthesis of Compound 140-c

To a solution of di(p-nitrophenyl) carbonate (2.07 g, 6.80 mmol) in acetonitrile (20 mL) was added a solution of tert-butyl L-alaninate (987 mg, 6.80 mmol) and DMAP (1.66 g, 13.59 mmol) in acetonitrile (20 mL) at room temperature. After addition, the reaction mixture was stirred at 50° C. under protection of nitrogen for 1 hour, was added compound 4 (500 mg, 0.85 mmol) and continued stirring at 50° C. under protection of nitrogen for 18 hours, was concentrated at reduced pressure. The residue was suspended in 100 mL of water, extracted with ethyl acetate (100 mL*2), the organic phase was washed sequentially with 10% NaHSO₄ solution (100 mL*2), brine (100 mL), dried over anhydrous sodium sulfate, filtered, the filtrate was concentrated at reduced pressure, and the residue was purified by a flash column chromatography (MeOH/DCM=0-10%). ) to afford compound 140-c (520 mg, 80%). LC-MS (ESI): m/z=760.5[M+H]⁺.

### Synthesis of Compound 140-b

Compound 140-c (520 mg, 0.68 mmol) was dissolved in 15 mL of DCM at room temperature, TFA (5 mL) was added, and the reaction mixture was stirred for 1 h at room temperature. The reaction mixture was concentrated at reduced pressure and the residue was dried under vacuum overnight to give the crude compound 140-b (715 mg). LC-MS (ESI): m/z=704.3[M+H]⁺.

### Synthesis of Compound 140-a

Compound 140-b (690 mg, 0.66 mmol) was dissolved in 10 mL of DMF at room temperature. Placed in an ice-water bath, the above mixture was added HATU (1.05 g, 2.77 mmol), DIPEA (665 uL, 4.03 mmol), and the stirred for 20 min in an ice-water bath, then was added di-tert-butyl (S)-2-amino glutarate (702 mg, 2.71 mmol). It was warmed to room temperature and continued stirring for 30 min. The reaction mixture was poured into 100 mL of water and the aqueous phase was extracted with ethyl acetate (100 mL). The organic phase was washed with brine (100 mL*5), dried over anhydrous sodium sulfate, filtered, and the filtrate was concentrated at reduced pressure, and the residue was purified by a flash column chromatography (MeOH/DCM=0-10%) to give compound 140-a (468 mg, 75%). LC-MS (ESI ): m/z=945.5[M+H]⁺.

### Synthesis of Compound 140

Compound 140-a (468 mg, 0.50 mmol) was dissolved in 6 mL of DCM at room temperature, TFA (2 mL) was added in an ice-water bath and the reaction mixture was stirred for 2 h at room temperature. The reaction mixture was concentrated at reduced pressure and the residue was purified by Prep-HPLC (TFA) to give compound 140 (320 mg, 60%). LC-MS (ESI): m/z=833.0[M+H]⁺; ¹H NMR (DMSO-d₆, 400 MHz): δ 10.89 (1H, brs), 10.32 (1H, s), 8.24-8.18 (3H, m), 7.97 (1H, d, J=1.2 Hz), 7.63 (1H, t, J=8.8 Hz), 7.44 (1H, t, J=2.8 Hz), 6.02-5.93 (1H, m), 5.56 (1H, d, J=52.4 Hz), 4.63-4.51 (2H, m), 4.25-4.09 (4H, m), 4.07-3.94 (2H, m), 3.91-3.67 (4H, m), 3.36-3.23 (1H, m), 3.11-3.01 (2H, m), 2.60-2.52 (2H, m), 2.48-2.42 (1H, m), 2.36-2.24 (3H, m), 2.24-2.10 (2H, m), 2.08-1.89 (4H, m), 1.84-1.58 (5H, m), 1.30 (3H, d, J=6.8 Hz).

### Synthetic Route of Compounds 141 and 142

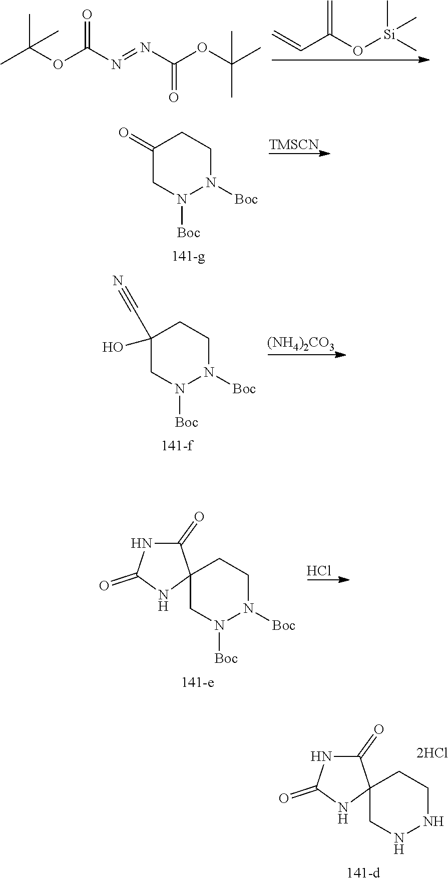

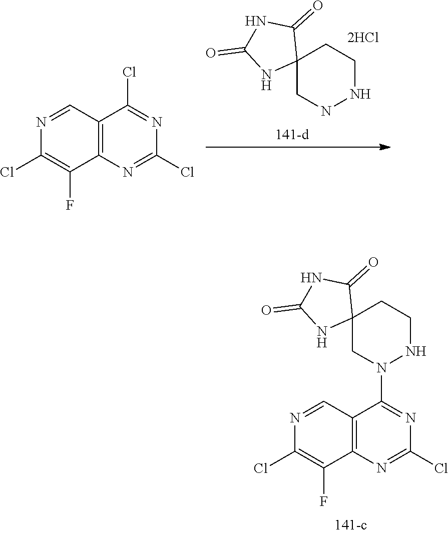 [SMILES: Fc1c(Cl)ncc2c(Cl)nc(Cl)nc12 ; O=C1NC(=O)C2(CCNNC2)N1 ; O=C1NC(=O)C2(CCNN(c3nc(Cl)nc4c(F)c(Cl)ncc34)C2)N1]

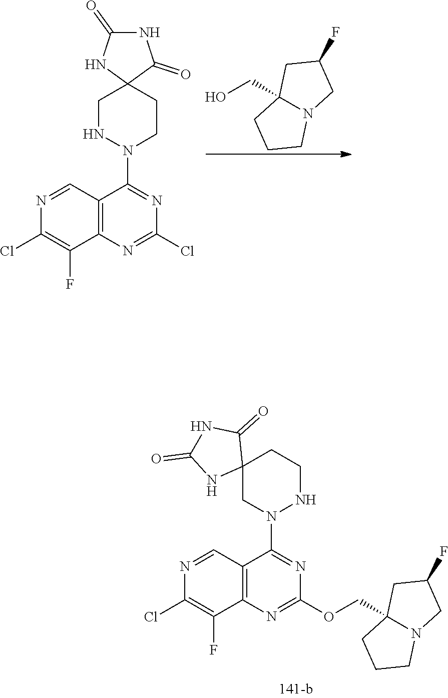

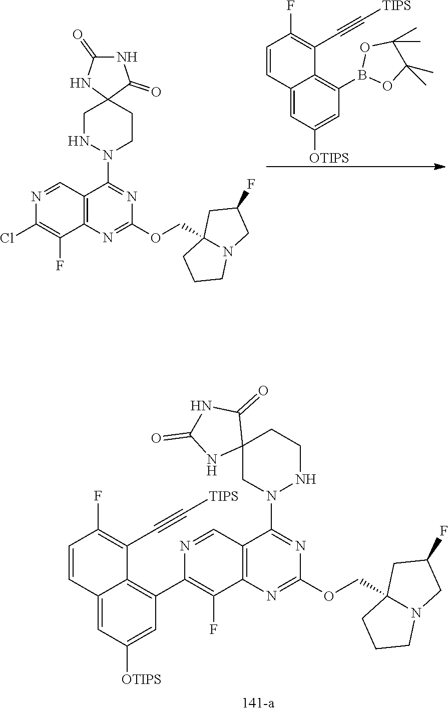

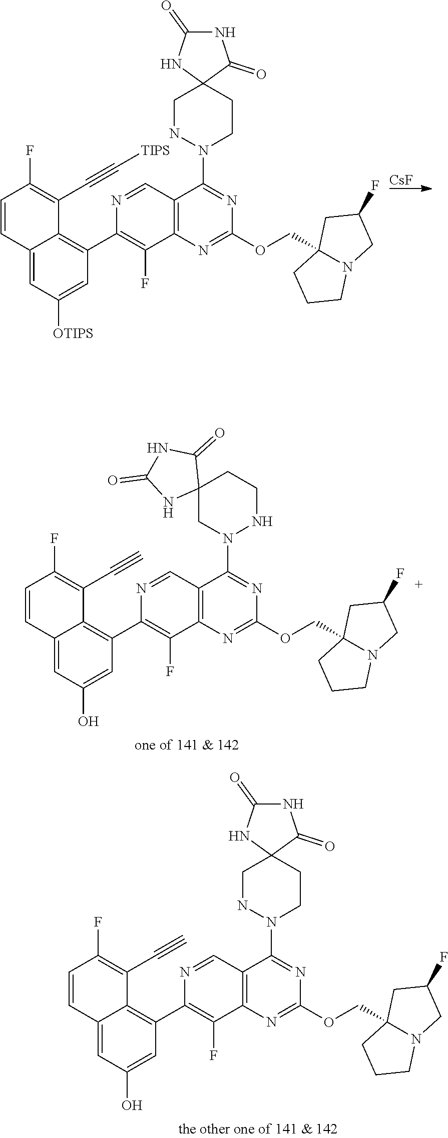

### Synthesis of Compound 141-g

2-Trimethylsiloxy-1,3-butadiene (5000 mg, 35.14 mmol), chloroform (50 mL), and di-tert-butyl azodicarboxylate (6200 mg, 26.93 mmol) were added sequentially to a reaction flask at room temperature. After degassed and purged with nitrogen, the mixture was stirred for 24 hours at room temperature. The reaction mixture was concentrated and the residue was purified by column chromatography (mobile phase: PE/EA 10/0 to 8/2) to give compound 141-g (950 mg, 12). LC-MS (ESI): m/z 318.3 (M+NH₄)+

### Synthesis of Compound 141-f

To a reaction flask were added 141-g (850 mg, 2.83 mmol), dimethyl sulfoxide (20 mL), water (4 mL), TMSCN (421 mg, 4.25 mmol), and the mixture was stirred for 2 hr at room temperature under protection of nitrogen. It was quenched with saturated sodium bicarbonate solution, and the aqueous phase was extracted twice with ethyl acetate. The organic phase was washed three times with brine, concentrated to dryness by rotary evaporation and the residue was purified by column chromatography (mobile phase: ethyl acetate: dichloromethane=0-20% ) to give 141-f (677 mg, 730). LC-MS (ESI): m/z 345.3 (M+NH₄)+

### Synthesis of Compound 141-e

To a reaction vial were added 141-f (600 mg, 1.83 mmol), ethanol (25 mL), ammonium carbonate (881 mg, 9.16 mmol), and the mixture was stirred at room temperature overnight under protection of nitrogen. Water was added to the reaction mixture and the aqueous phase was extracted three times with ethyl acetate. The organic phase was washed with a small amount of brine, concentrated to dryness by rotary evaporation, and the residue was purified by column chromatography (mobile phase: methanol: dichloromethane=0-5%) to give 141-e (560 mg, 82%). LC-MS (ESI): m/z 371.2 (M+H)⁺.

### Synthesis of Compound 141-d

To a reaction vial were added 141-e (560 mg, 1.51 mmol), acetonitrile (20 mL), hydrochloride/1,4-dioxane (4 mL, 16 mmol) and the mixture was stirred at room temperature overnight. The reaction mixture was concentrated to dryness by rotary evaporation to give 141-d (521 mg, 142%), which was used directly in the next step without purification. LC-MS (ESI): m/z 171.2 (M+H)⁺.

### Synthesis of Compound 141-c

To a reaction vial were added 141-d (250 mg), 2,4,7-trichloro-8-fluoropyrido[4,3-d]pyrimidine (215 mg, 0.85 mmol), dichloromethane (50 mL). Cooled in a dry ice-acetone bath, the above mixture was added DIPEA (0.99 mL, 5.96 mmol) and warmed naturally to room temperature under protection of nitrogen and stirred for 2 hours. The reaction mixture was added water, filtered and the filter cake was dried in vacuum; the filtrate was extracted five times with dichloromethane, dried over anhydrous sodium sulfate, concentrated to dryness by rotary evaporation and the residue was purified by column chromatography (mobile phase: methanol: dichloromethane=0-10%), together with the previous filter cake were combined to give 141-c (327 mg, 99%). LC-MS (ESI): m/z 386.1(M+H)⁺.

### Synthesis of Compound 141-b

To a reaction vial were added 141-c (314 mg, 0.81 mmol), dried 1,4-dioxane (60 mL), ((2R,7aS)-2-fluorohexahydro-1H-pyrrolozin-7a-yl)methanol (388 mg, 2.44 mmol), DIPEA (525 mg, 4.07 mmol) and the mixture was stirred at 90° C. under protection of nitrogen for 3 days. The reaction mixture was concentrated to dryness by rotary evaporation and the residue was purified by column chromatography (mobile phase: methanol: dichloromethane=0-10%) to give 141-b (231 mg, 56%).

LC-MS (ESI): m/z 509.2 (M+H)⁺.

### Synthesis of Compound 141-a

To a microwave tube was added 141-b (160 mg, 0.31 mmol), ((6-fluoro-4-(4,4,5,5-tetramethyl-1,3,2-dioxaborolan-2-yl)-5-((triisopropylmethylsilyl)ethynyl)naphthalen-2-yl)oxy)triisopropylsilane (393 mg, 0.63 mmol), potassium phosphate (334 mg, 1.57 mmol), cataCXium A Pd G3 (23 mg, 0.031 mmol), tetrahydrofuran (15 mL) and water (2 mL). After sealed, degassed and purged with nitrogen for three times, the reaction mixture was heated at 75° C. for 2 hours. The reaction mixture was concentrated to dryness by rotary evaporation and the residue was purified by column chromatography (mobile phase: methanol: dichloromethane=0-100%) to give 141-a (230 mg, 75%). LC-MS (ESI): m/z 971.7 (M+H)⁺.

### Synthesis of Compounds 141 and 142

Cesium fluoride (1079 mg, 7.10 mmol) was added to a solution of 141-a (230 mg, 0.24 mmol) in DMF (10 mL) at room temperature and the mixture was stirred at room temperature overnight. The next day, the reaction mixture was filtered, purified by prep-HPLC (ammonium bicarbonate), lyophilized to give 141 (53 mg, 34%). LC-MS (ESI): m/z 659.2 (M+H)⁺; ¹H NMR (400M, DMSO-d₆): δ 10.90 (1H, s), 10.26 (1H, s), 10.15 (1H, s), 8.82-8.54 (1H, m), 8.05-7.94 (1H, m), 7.47 (1H, t, J=8.8 Hz), 7.39 (1H, d, J=2.4 Hz), 7.18(1H, dd, J=2.4 Hz, J=18.8 Hz), 5.87-5.72 (1H, m), 5.27 (1H, d, J=54.0 Hz), 4.19-3.66 (4H, m), 3.31-3.17 (2H, m), 3.16-2.94 (4H, m), 2.87-2.76 (1H, m), 2.20-1.94 (4H, m), 1.89-1.64 (4H, m). 142 (6 mg, 4%), LC-MS (ESI): m/z 659.2 (M+H)⁺.

### Synthetic Route of Compound 143

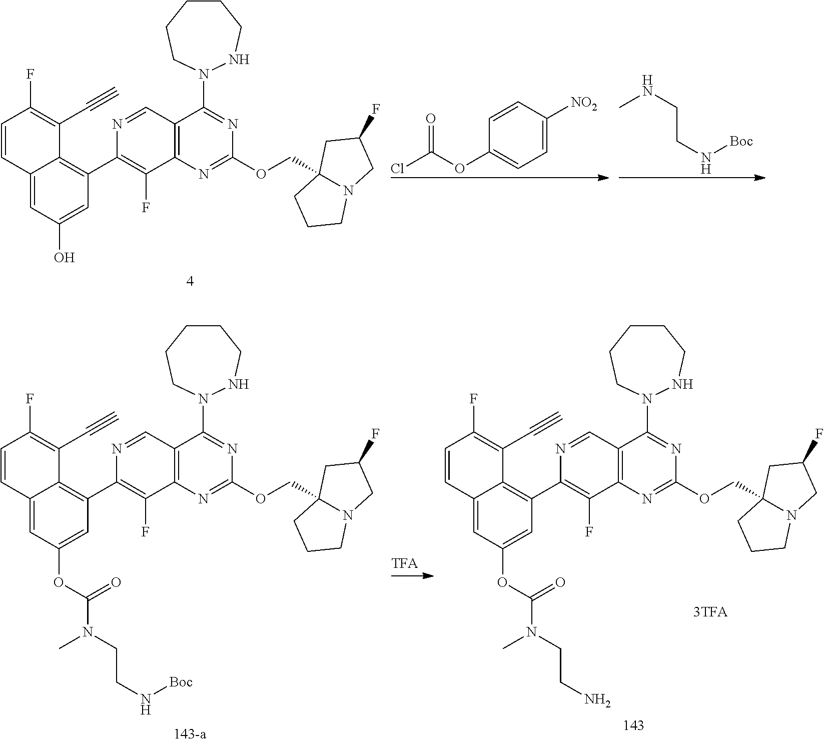

### Synthesis of Compound 143-a

To a solution of compound 4 (200 mg, 0.34 mmol) in DCM (10 mL) was added triethylamine (0.094 mL, 0.68 mmol) and p-nitrophenyl chloroformate (75 mg, 0.37 mmol) in an ice-water bath. The reaction mixture was stirred at this temperature for 2 hours, then was added tert-butyl 2-(methylamino)ethylcarbamate (178 mg, 1.02 mmol) and the reaction mixture was slowly warmed to room temperature and stirred at this temperature overnight. Water was added to the reaction mixture and the aqueous phase was extracted with dichloromethane (40 mL*2). The combined organic phases were dried over anhydrous sodium sulfate, filtered, concentrated to dryness by rotary evaporation and the residue was purified by column chromatography (mobile phase: methanol/dichloromethane, 0-10%) to give 143-a (237 mg, 88%). LC-MS (ESI): m/z 789.6 (M+H)⁺.

### Synthesis of Compound 143

Trifluoroacetic acid (4 mL) was added to a solution of 143-a (237 mg, 0.30 mmol) in dichloromethane (12 mL) at room temperature and the reaction mixture was stirred for 1 h at room temperature under protection of nitrogen. Concentrated to dryness by rotary evaporation at room temperature, and the residue was purified by prep-HPLC (trifluoroacetic acid) and lyophilized to give trifluoroacetate 143 (233 mg, 75%). LC-MS (ESI): m/z 689.3(M+H)⁺; ¹H NMR (400M, DMSO-d₆) δ 10.98(1H, bs), 10.32 (1H, s), 8.26-8.15 (1H, m), 8.07-7.79 (4H, m), 7.69-7.56 (2H, m), 5.99 (1H, s), 5.57 (1H, d, J=52.8 Hz), 4.64-4.50 (2H, m), 4.21-3.96 (4H, m), 3.92-3.66 (4H, m), 3.59-3.50 (1H, m), 3.38-3.24 (1H, m), 3.19-2.92 (7H, m), 2.37-1.85 (8H, m), 1.78-1.58 (4H, m).

### Synthetic Route of Compound 144

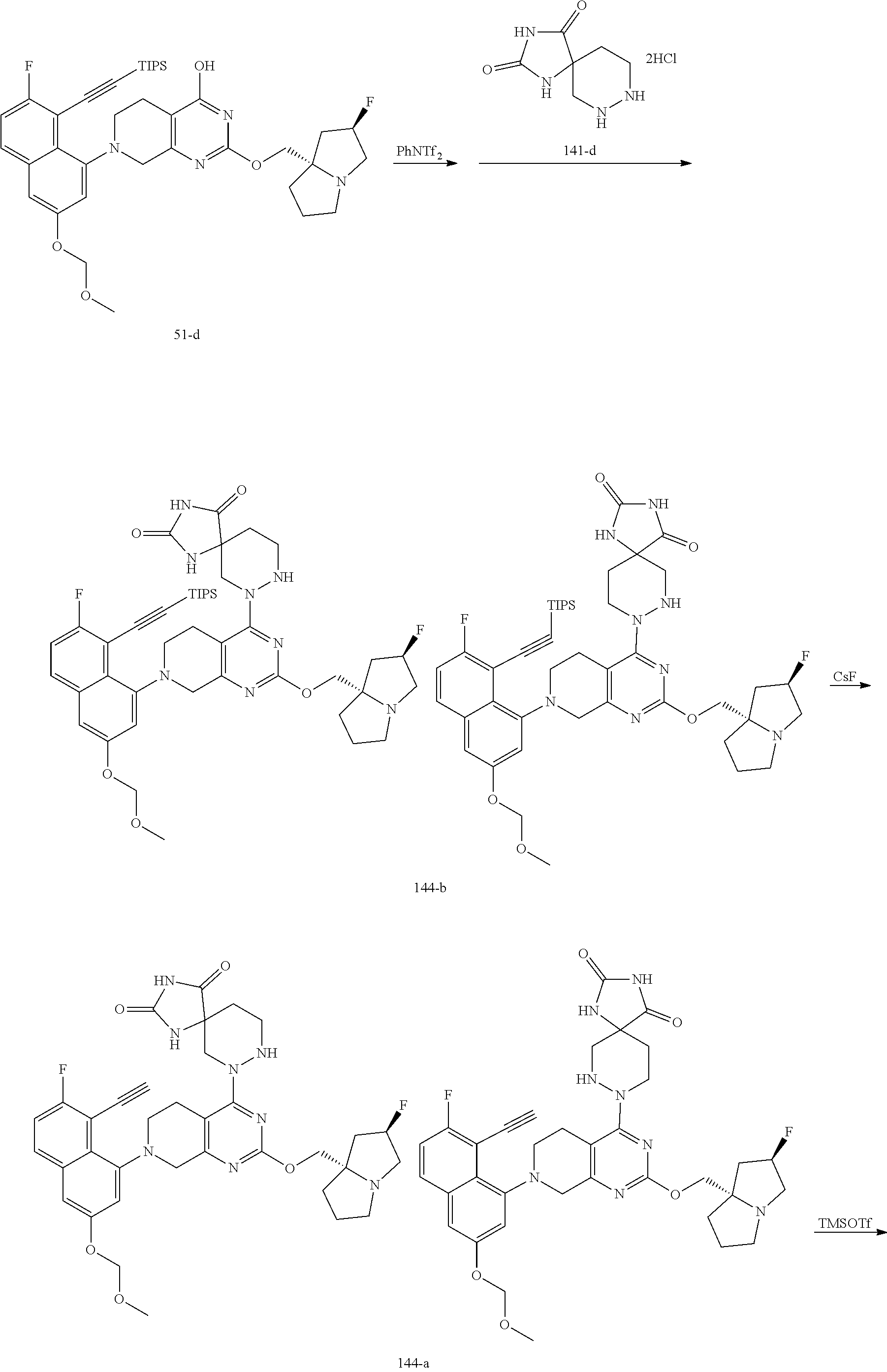

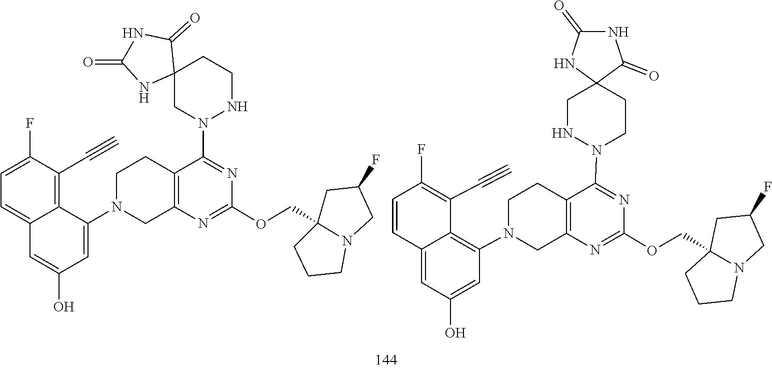

### Synthesis of Compound 144-b

N-phenylbis(trifluoromethanesulfonyl)imide (68 mg, 0.19 mmol) and DIPEA (47 uL, 0.29 mmol) were added to a solution of compound 51-d (66 mg, 0.095 mmol) in 2 mL of anhydrous DMSO at room temperature. The reaction mixture was stirred for 2 h at room temperature under protection of nitrogen, then was added a solution of compound 141-d (65 mg, 0.19 mmol) and DIPEA (47 uL, 0.28 mmol) in anhydrous DMSO (1 mL). After addition, it was stirred at 70° C. for 1 hour. The reaction mixture was cooled to room temperature, quenched with water, extracted with ethyl acetate (50 mL*2). The organic phase was washed with brine (100 mL*5), dried over anhydrous sodium sulfate, filtered, and the filtrate was concentrated at reduced pressure, and the residue was purified by a flash column chromatography (MeOH/DCM=0-10%) to afford compound 144-b (42 mg, 52%). LC-MS (ESI): m/z=845.5[M+H]⁺.

### Synthesis of Compound 144-a

Compound 144-b (42 mg, 0.050 mmol) was dissolved in 3 mL of DMF at room temperature, cesium fluoride (204 mg, 1.34 mmol) was added, and the reaction mixture was stirred at room temperature under protection of nitrogen for 1 hour. The reaction mixture was poured into 20 mL of water, extracted with ethyl acetate (50 mL*2). The organic phase was washed with brine (100 mL*5), dried over anhydrous sodium sulfate, filtered, and the filtrate was concentrated at reduced pressure and dried under vacuum to obtain the crude compound 144-a (34 mg), and the crude product was used directly in the next step. LC-MS (ESI): m/z=689.4 [M+H]⁺.

### Synthesis of Compound 144

Compound 144-a (34 mg, 0.049 mmol) was dissolved in 2 mL of DCM at room temperature, trimethylsilyl trifluoromethanesulfonate (200 uL, 1.10 mmol) was added in an ice-water bath, and the reaction mixture was stirred in an ice-water bath under protection of nitrogen for 1 hr. To the reaction mixture was added 50 mL of saturated sodium bicarbonate solution, and the aqueous phase was extracted with mixed solvents (DCM/MeOH=10:1, 50 mL*3). The organic phase was dried over anhydrous sodium sulfate, filtered, and the filtrate was concentrated at reduced pressure, and the residue was purified by prep-HPLC (alkaline method, NH₄HCO₃ system) to give compound 144 (6.6 mg, 21%). LC-MS (ESI): m/z=645.4[M+H]⁺.

### Synthetic Route of Compound 145

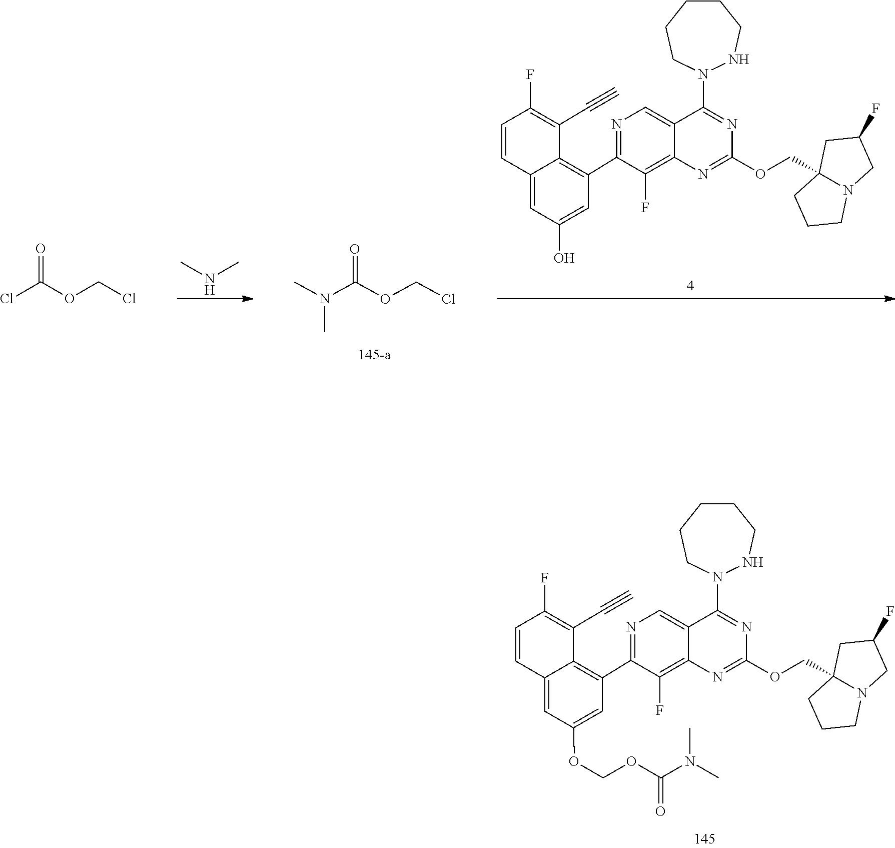

### Synthesis of Compound 145-a

Chloromethyl chloroformate (1000 mg, 7.76 mmol) and tetrahydrofuran (10 mL) were added to a reaction flask separately. The mixture was stirred in an ice-salt bath under protection of nitrogen for 10 min, then was added a solution of dimethylamine (0.514 mL, 2M, 7.76 mmol)in tetrahydrofuran, and the reaction mixture was slowly warmed to room temperature under an ice-salt bath and stirred under protection of nitrogen for 2 hours. The reaction mixture was filtered and the filtrate was concentrated to give compound 145-a (230 mg, 22%), which was directly in the next step.

### Synthesis of Compound 145

Compound 4 (200 mg, 0.34 mmol), 145-a (230 mg, 1.67 mmol), potassium carbonate (141 mg, 1.02 mmol), acetonitrile (30 mL), tetrabutylammonium iodide (25 mg, 0.07 mmol), and water (122 mg, 6.80 mmol) were added to a reaction vial and stirred for 6 hours at room temperature. The reaction mixture was concentrated and the residue was purified by column chromatography (DCM/MeOH=100/0; 100/10) to give compound 145 (68 mg, 29%). LC-MS (ESI): m/z=690.0(M+H)⁺; ¹H NMR (400 MHz, DMSO-d₆): δ 10.26 (1H, s), 8.15 (1H, dd, J₁=9.2 Hz, J₂=3.2 Hz), 7.83 (1H, d, J=2.4 Hz), 7.57 (1H, d, J=9.2 Hz), 7.38 (1H, d, J=2.0 Hz), 5.91 (2H, s), 5.94-5.82 (1H, m), 5.34 (1H, d, J=54.8 Hz), 4.38-3.93 (4H, m), 3.98 (1H, s), 3.14-3.00 (2H, m), 2.78-2.96 (2H, m), 2.86 (6H, d, J=9.2 Hz), 2.30-2.00 (4H, m), 1.98-1.78 (6H, m), 1.76-1.60 (4H, m).

### Synthesis of Compound 146

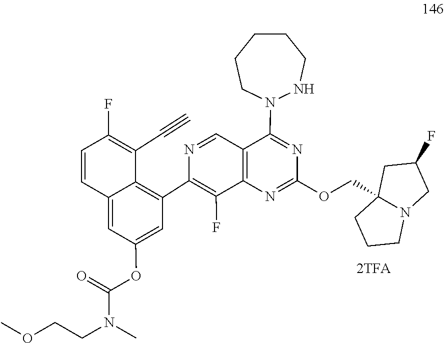 [SMILES: C#Cc1c(F)ccc2cc(OC(=O)N(C)CCOC)cc(-c3ncc4c(N5CCCCCN5)nc(OC[C@@]56CCCN5C[C@H](F)C6)nc4c3F)c12]

Referring to the synthetic route of compound 97, compound 146 was obtained by using N-(2-methoxyethyl)methylamine instead of N-cyclopropyl-N-ethylamine and prep-HPLC purification (TFA). LC-MS (ESI): m/z 704.2 (M+H)⁺; ¹H NMR (400 MHz, DMSO-d₆): δ 10.93 (1H, s), 10.33 (1H, s), 8.22 (1H, dd, J=8.8, 6 Hz), 8.12 (0.5H, d, J=9.2 Hz), 8.00 (1H, d, J=8.8 Hz), 7.65 (1H, t, J=8.8 Hz), 7.50 (1H, d, J=8.8 Hz), 6.94 (0.5H, d, J=9.2 Hz), 5.99 (1H, s), 5.57 (1H, d, J=52.8 Hz), 4.65-4.51 (3H, m), 4.20-4.09 (1H, m), 4.07-3.97 (2H, m), 3.91-3.72 (3H, m), 3.66-3.57 (2H, m), 3.50 (2H, dd, J=13.6, 4.8 Hz), 3.30 (4H, d, J=10 Hz), 3.14-3.03 (3H, m), 2.97 (1H, s), 2.64-2.52 (2H, m), 2.36-2.29 (1H, m), 2.23-2.12 (2H, m), 2.09-2.00 (1H, m), 1.95 (2H, s), 1.71 (4H, s).

### Synthetic Route of Compound 147

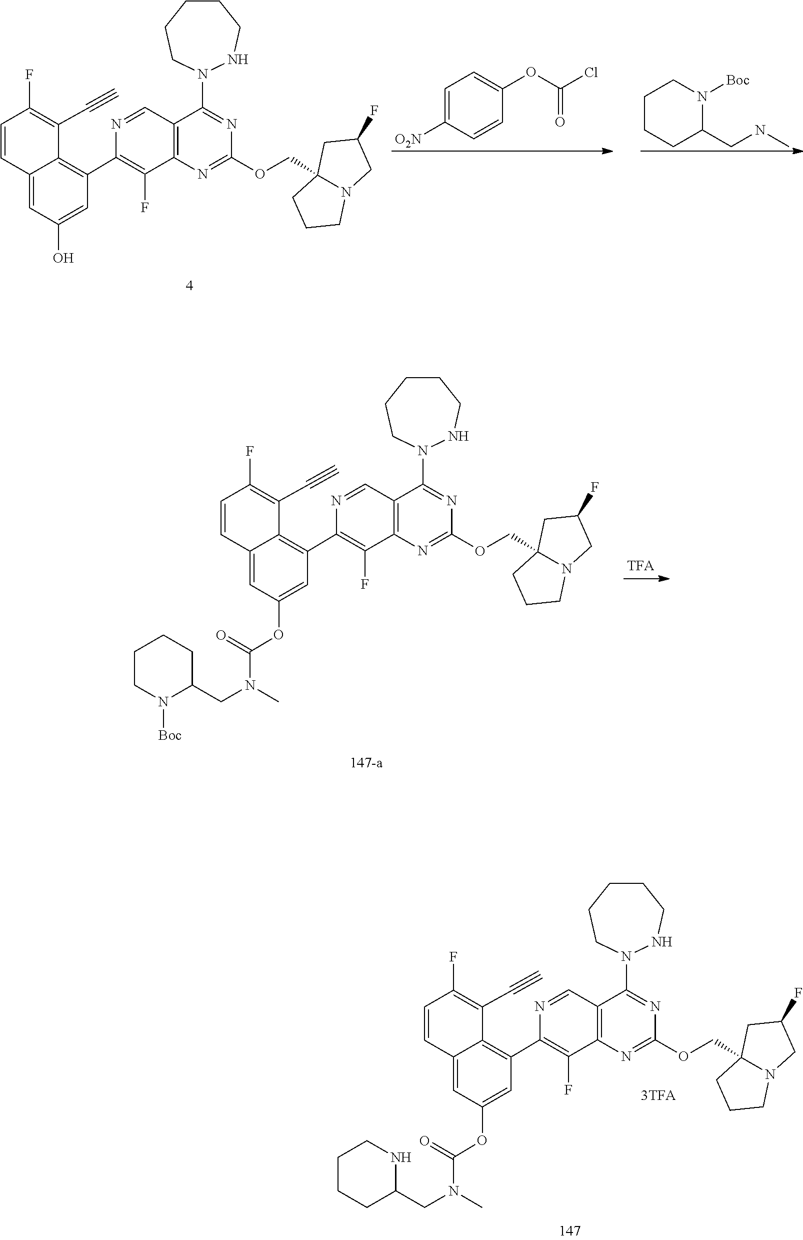 [SMILES: C#Cc1c(F)ccc2cc(O)cc(-c3ncc4c(N5CCCCCN5)nc(OC[C@@]56CCCN5C[C@H](F)C6)nc4c3F)c12 ; O=C(Cl)Oc1ccc([N+](=O)[O-])cc1 ; CNCC1CCCCN1C(=O)OC(C)(C)C ; C#Cc1c(F)ccc2cc(OC(=O)N(C)CC3CCCCN3C(=O)OC(C)(C)C)cc(-c3ncc4c(N5CCCCCN5)nc(OC[C@@]56CCCN5C[C@H](F)C6)nc4c3F)c12 ; C#Cc1c(F)ccc2cc(OC(=O)N(C)CC3CCCCN3)cc(-c3ncc4c(N5CCCCCN5)nc(OC[C@@]56CCCN5C[C@H](F)C6)nc4c3F)c12]

### Synthesis of Compound 147-a

To a solution of compound 4 (200 mg, 0.34 mmol) in dichloromethane (8 mL) was added triethylamine (0.094 mL, 0.68 mmol) and p-nitrophenyl chloroformate (103 mg, 0.51 mmol) in an ice water bath. The reaction mixture was stirred at this temperature for 2 hours, then was added tert-butyl 2-[(methylamino)methyl]-1-piperidinecarboxylate (155 mg, 0.68 mmol) and was slowly warmed to room temperature and stirred at this temperature overnight. Water was added and the aqueous phase was extracted with dichloromethane (40 mL). The organic phase was dried over anhydrous sodium sulfate, filtered, concentrated to dryness by rotary evaporation, and the crude was purified by flash column chromatography (mobile phase: dichloromethane: methanol=10:1/dichloromethane, 0-100%) to give 147-a (266 mg, 93%). LC-MS (ESI): m/z 843.9 (M+H)⁺.

### Synthesis of Compound 147

To a solution of 147-a (266 mg, 0.32 mmol) in dichloromethane (8 mL) was added trifluoroacetic acid (1 mL) at room temperature and the mixture was stirred for 2 h at this temperature. The reaction mixture was concentrated to dryness by rotary evaporation and the crude was purified by prep-HPLC (acid method) to give 147 (226 mg, 96%). LC-MS (ESI): m/z 743.2 (M+H)⁺; ¹H NMR (400 MHz, DMSO-d₆): δ 11.05 (1H, s), 10.32 (1H, s), 8.81 (1H, dd, J=8.8, 6 Hz), 8.58-8.36 (1H, m), 8.20 (1H, dd, J=9.2, 6 Hz), 8.01 (1H, d, J=2 Hz), 7.69-7.60 (2H, m), 6.00 (1H, s), 5.58 (1H, d, J=52.4 Hz), 4.64-4.51 (4H, m), 4.21-4.09 (2H, m), 4.07-3.97 (3H, m), 3.94-3.59 (5H, m), 3.55-3.24 (4H, m), 3.17-2.96 (5H, m), 2.95-2.83 (1H, m), 2.64-2.52 (1H, m), 2.35-2.27 (1H, m), 2.25-2.11 (2H, m), 2.09-2.01 (1H, m), 1.99-1.91 (2H, m), 1.86-1.65 (7H, m).

### Synthetic Route of Compound 148

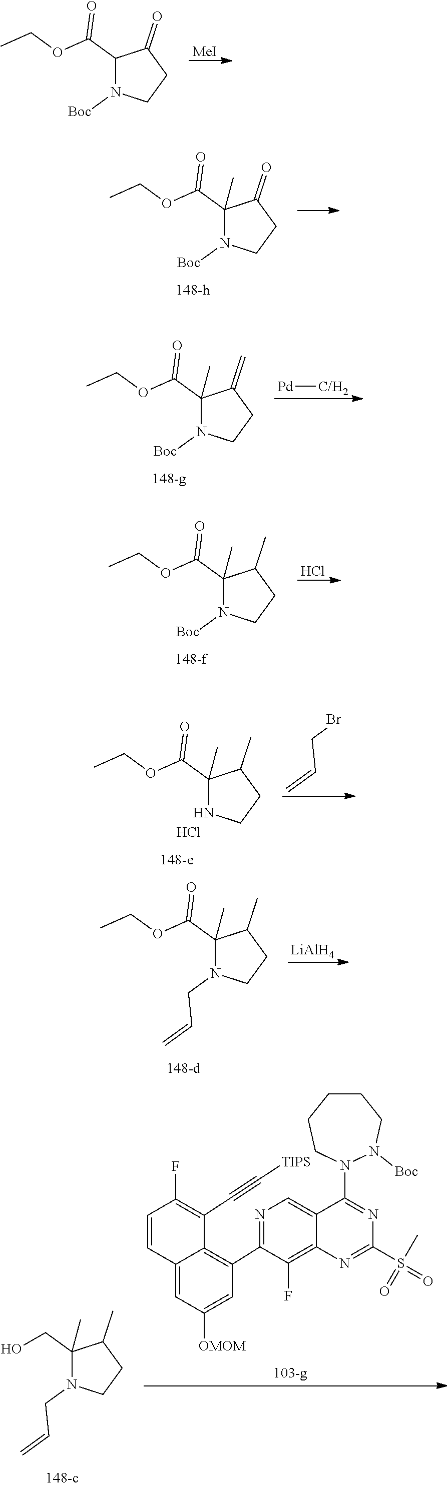

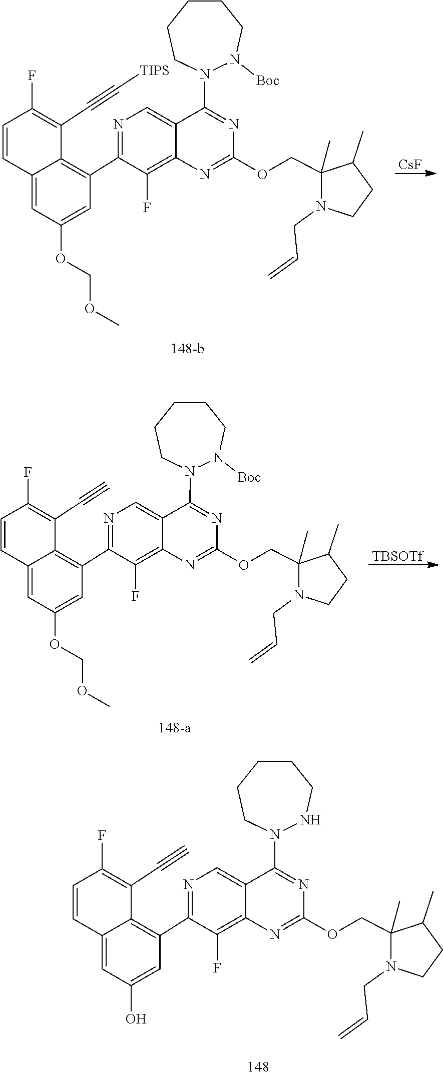

### Synthesis of Compound 148-h

Compound N—BOC-3-oxopyrrolidine-2-carboxylic acid ethyl ester (5 g, 19.43 mmol) was dissolved in acetonitrile (50 mL), to which potassium carbonate (3.22 g, 23.30 mmol), iodomethane (6.9 g, 48.61 mmol) were added, and the reaction mixture was stirred at 45° C. overnight. The reaction mixture was filtered, and the filtrate was concentrated under reduced pressure and purified by column chromatography (mobile phase, PE/EA 10/1) to afford compound 148-h (2.6 g, 49%). LC-MS (ESI): m/z 216.2(M+H-tBu)⁺.

### Synthesis of Compound 148-g

Tert-butanol potassium tetrahydrofuran solution (1M, 5.53 mL, 5.53 mmol) was added dropwise to a solution of compound methyltriphenylphosphonium bromide (1.98 g, 5.54 mmol) in anhydrous toluene (15 mL) in an ice-water bath, and the reaction mixture was stirred for 30 min in an ice-water bath. To the reaction mixture was added a solution of 148-h (1 g, 3.69 mmol) in anhydrous toluene (2 mL), the reaction mixture was warmed to room temperature and stirred for 1 h. The reaction mixture was placed in an oil bath at 110° C. and stirred overnight. The reaction mixture was cooled and concentrated, and the residue was purified by column chromatography (mobile phase, PE/EA 3/1) to give compound 148-g (650 mg, 65%). LC-MS (ESI): m/z 270.3 (M+H)⁺.

### Synthesis of Compound 148-f

Compound 148-g (650 mg, 2.41 mmol) was dissolved in ethanol (20 mL), to which wet palladium carbon (500 mg, 10%) was added under protection of nitrogen, and the reaction mixture was degassed and purged with hydrogen three times before stirring at room temperature at atmosphere of hydrogen overnight. The reaction mixture was filtered through celite and the filtrate was concentrated underreduced pressure and purified by column chromatography (mobile phase, PE/EA 3/1) to give compound 148-f (630 mg, 96%). LC-MS (ESI): m/z 272.3 (M+H)⁺.

### Synthesis of Compound 148-e

Compound 148-f (630 mg, 2.32 mmol) was dissolved in ethyl acetate (6 mL), to which hydrochloride /1,4-dioxane (4 M, 10 mL) was added, and the reaction mixture was stirred for 2 h at room temperature. The reaction mixture was concentrated under reduced pressure. The residue was added ethyl acetate (20 mL) and stirred at room temperature for 30 min, the solid was filtered and dried to give compound 148-e (428 mg, 89%). LC-MS (ESI): m/z 172.3 (M+H)⁺.

### Synthesis of Compound 148-d

Compound 148-e (428 mg, 2.06 mmol) was added to acetonitrile (15 mL), to which potassium carbonate (1.14 g, 8.25 mmol), 3-bromopropene (500 mg, 4.13 mmol) were added, and the reaction mixture was stirred at 80° C. overnight. Water (100 mL) was added to the reaction mixture and the aqueous phase was extracted with ethyl acetate (100 mL). The organic phase was washed with brine (50 mL), dried over sodium sulfate, filtered off the desiccant, and the filtrate was concentrated under reduced pressure and purified by column chromatography (mobile phase, PE/EA 10/1) to give compound 148-d (340 mg, 78%). LC-MS (ESI): m/z 212.3(M+H)⁺.

### Synthesis of Compound 148-c

Compound 148-d (340 mg, 1.61 mmol) was dissolved in anhydrous tetrahydrofuran (10 mL). After degassed and purged with nitrogen for 3 times, the above mixture was added a solution of lithium tetrahydroaluminum in tetrahydrofuran (1 M, 2.41 mL, 2.41 mmol) dropwise in an ice-water bath. After addition, the reaction mixture was stirred in an ice-water bath for 1 hour. The reaction mixture was added 1 g of solid sodium sulfate decahydrate, and stirred for 10 min, then filtered through celite, the filter cake was drenched with ethyl acetate (50 mL), and the filtrate was concentrated and dried under reduced pressure to give 148-c (164 mg, 60%). LC-MS (ESI): m/z 170.3 (M+H)⁺.

### Synthesis of Compound 148-b

Compounds 103-g (200 mg, 0.25 mmol) and 148-c (96 mg, 0.57 mmol) were dissolved in dichloromethane (10 mL). Placed in an ice-water bath, the mixture was added sodium tert-butanol (48 mg, 0.50 mmol), and the reaction mixture was stirred for 1 hr in an ice-water bath. Water (50 mL) was added to the reaction mixture and the aqueous phase was extracted with dichloromethane (50 mL). The organic phase was washed with brine (50 mL), dried over sodium sulfate, filtered off the desiccant, and the filtrate was concentrated under reduced pressure and purified by column chromatography (mobile phase, DCM/MeOH 20/1) to give compound 148-b (80 mg, 36%). LC-MS (ESI): m/z 899.1 (M+H)⁺.

### Synthesis of Compound 148-a

Compound 148-b (80 mg, 0.089 mmol) was dissolved in N,N-dimethylformamide (6 mL), to which cesium fluoride (135 mg, 0.89 mmol) was added and the reaction mixture was stirred at room temperature overnight. Water (100 mL) was added to the reaction mixture and the aqueous phase was extracted with ethyl acetate (50 mL). The organic phase was washed with brine (50 mL), dried over sodium sulfate, filtered off the desiccant, and the filtrate was concentrated under reduced pressure and purified by column chromatography (mobile phase, DCM/MeOH 20/1) to give compound 148-a (45 mg, 68%). LC-MS (ESI): m/z 743.4 (M+H)⁺.

### Synthesis of Compound 148

Compound 148-a (45 mg, 0.061 mmol) was dissolved in dichloromethane (3 mL), to which tert-butyldimethylsilyl trifluoromethanesulfonate (0.3 mL) was added in an ice-water bath, and the reaction mixture was stirred for 2 h at room temperature. 10% sodium bisulfate solution (3 mL) was added to the reaction mixture and continued stirring for 2 h. Saturated sodium bicarbonate solution was added to the mixture to neutralize it, and it was extracted with dichloromethane (50 mL). The organic phase was washed with brine (50 mL), dried over sodium sulfate, filtered off the desiccant, and the filtrate was concentrated under reduced pressure, and the crude was purified by prep-HPLC (basic conditions) to give compound 148 (5 mg, 214). LC-MS (ESI): m/z 599.4(M+H)⁺.

### Synthetic Route of Compound 149

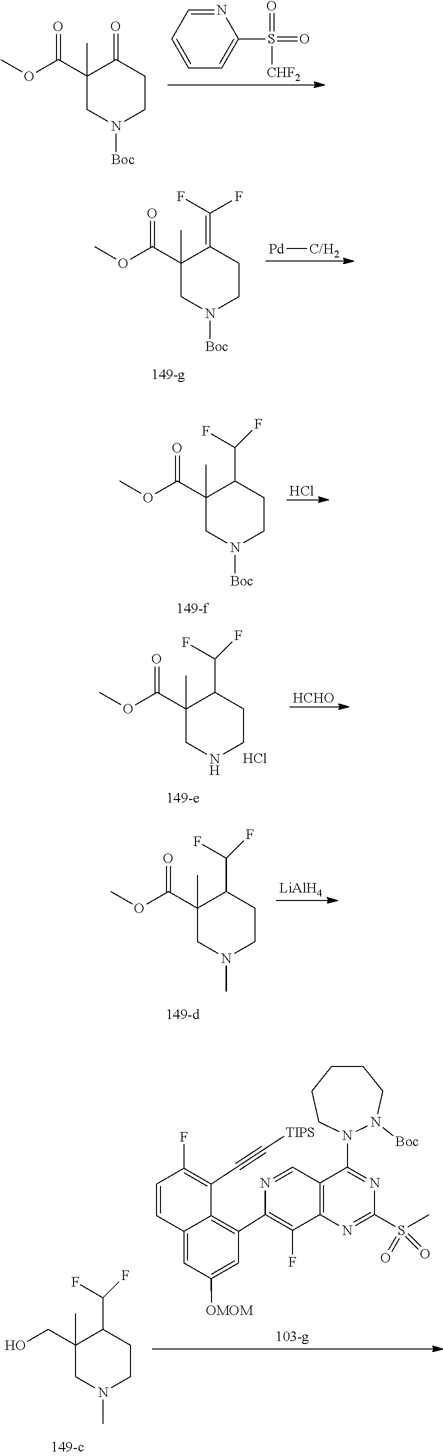

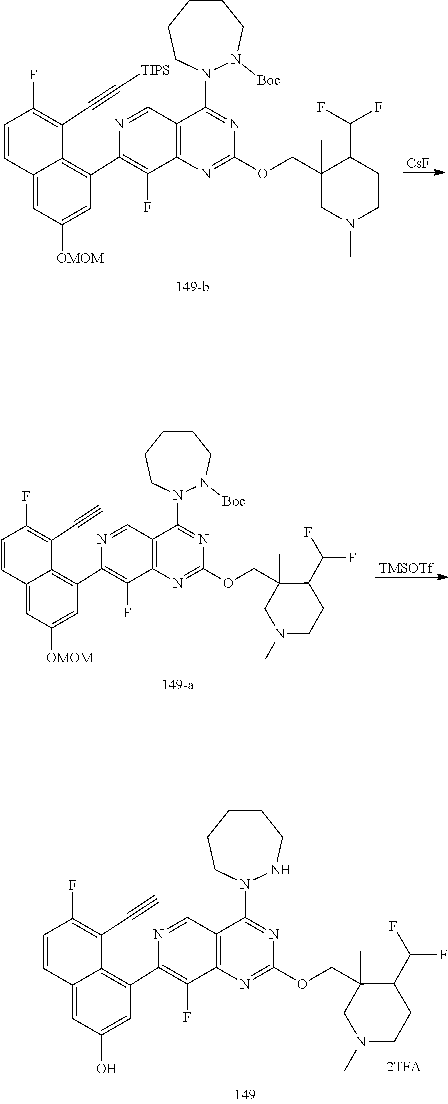

### Synthesis of Compound 149-g

To a reaction vial at room temperature was added 1-tert-butyl-3-methyl-4-oxopiperidine-1,3-dicarboxylic acid-3-methyl ester (1000 mg, 3.69 mmol), DMF (15 mL), and difluoromethyl (2-pyridyl) sulfone (1068 mg, 5.53 mmol). After degassed and purged with N2 and placed in an dry ice- acetone bath, the mixture was added a solution of potassium tert-butanolate in tetrahydrofuran (1M, 6.63 mg, 6.63 mmol) dropwise. After addition, the reaction mixture continued stirring in an dry ice- acetone bath for 2 h, and then naturally warmed to room temperature and stirred overnight. Saturated aqueous ammonium chloride solution was added to the reaction mixture and the aqueous phase was extracted with EA. The organic phases were combined, washed with water, brine, dried over anhydrous sodium sulfate, concentrated, and purified by column purification (mobile phase: PE/EA 10/0 to 8/2) to give compound 149-g (420 mg, 37%). LC-MS (ESI): m/z 306.2 (M+H)⁺.

### Synthesis of Compound 149-f

To a reaction flask at room temperature were added 149-g (420 mg, 1.38 mmol), methanol (5 mL) and 10% palladium carbon (200 mg). After degassed and purged with hydrogen, the mixture was stirred protected by hydrogen for 3 hours at room temperature. The reaction mixture was filtered and concentrated to give the crude compound 149-f (460 mg). LC-MS (ESI): m/z 308.2 (M+H)⁺.

### Synthesis of Compound 149-e

To a reaction flask at room temperature was added crude 149-f (460 mg, 1.38 mmol), hydrogen chloride/1,4-dioxane (4M, 5 mL, 20 mmol). After degassed and purged with N2, the mixture was stirred protected by nitrogen for 1 hour at room temperature. The reaction mixture was concentrated to give the crude compound 149-e (350 mg). LC-MS (ESI): m/z 208.3 (M+H)⁺.

### Synthesis of Compound 149-d

To a reaction flask at room temperature was added crude 149-e (350 mg, 1.38 mmol) and methanol (5 mL). After degassed and purged with N2, the mixture was added aqueous formaldehyde solution (37%, 1.5 mL) and stirred for at room temperature 1 h. Sodium cyanoborohydride (173 mg, 2.75 mmol) was added and the reaction mixture was stirred at room temperature for 1 hour. The reaction mixture was concentrated, was added water and the aqueous phase was extracted with EA. The organic phases were combined, washed with water, brine, dried over anhydrous sodium sulfate, concentrated, and purified by column chromatography (mobile phase: PE/EA 10/0 to 0/10) to give compound 149-d (240 mg, 79%). LC-MS (ESI): m/z 222.3 (M+H)⁺.

### Synthesis of Compound 149-c

To a reaction vial at room temperature was added 149-d (240 mg, 1.09 mmol), THF (5 mL). After degassed and purged with N2, the mixture was added a solution of lithium aluminum hydride in tetrahydrofuran (4 M, 1.6 mL, 1.6 mmol) dropwise in an ice-water bath, and continued stirring for 1 hr after the addition. The reaction mixture was added sodium sulfate decahydrate until no bubbles were produced, followed by addition of a small amount of THF, dried over anhydrous sodium sulfate. The reaction mixture was filtered through celite and the filtrate was concentrated to give compound 149-c (160 mg, 76%). LC-MS (ESI): m/z 194.2 (M+H)⁺.

### Synthesis of Compound 149-b

To a reaction flask at room temperature was added 103-g (503 mg, 0.62 mmol) and toluene (5 mL). After degassed and purged with N2, the mixture was added 149-c (80 mg, 0.41 mmol), sodium tert-butoxide (119 mg, 1.24 mmol) under protection of N2 in an ice-water bath. The reaction mixture was continued stirring in an ice-water bath for 2 hours, then was purified directly by column chromatography (mobile phase: PE/EA 10/0 to 0/10) to give compound 149-b (150 mg, 39%). LC-MS (ESI): m/z 923.7 (M+H)⁺.

### Synthesis of compound 149-a

To a reaction vial at room temperature was added 149-b (150 mg, 0.16 mmol), DMF (2 mL). After degassed and purged with N2, the mixture was added cesium fluoride (123 mg, 0.81 mmol), heated to 50° C., and stirred for 1 hour. Water was added to the reaction mixture and the aqueous phase was extracted with EA. The organic phases were combined, washed with water, brine, dried over anhydrous sodium sulfate and concentrated to give the crude compound 149-a (150 mg). LC-MS (ESI): m/z 767.7 (M+H)⁺.

### Synthesis of Compound 149

To a reaction vial at room temperature was added crude compound 149-a (150 mg, 0.16 mmol) and DCM (3 mL). After degassed and purged with N2, and cooled down to 0° C., the mixture was added trimethylsilyl trifluoromethanesulfonate (0.3 mL) dropwise, then stirred for 1 hour. It was added 10% aqueous sodium bisulfate (1 mL) and stirred for 1 hour. The reaction mixture was adjusted basic with saturated aqueous sodium bicarbonate and the aqueous phase was extracted with DCM. The organic phases were combined, washed with water, brine, dried, concentrated and purified by prep-HPLC to give compound 149 (39 mg, 28%). LC-MS (ESI): m/z 623.2 (M+H)⁺.

### Synthetic Route of Compound 150

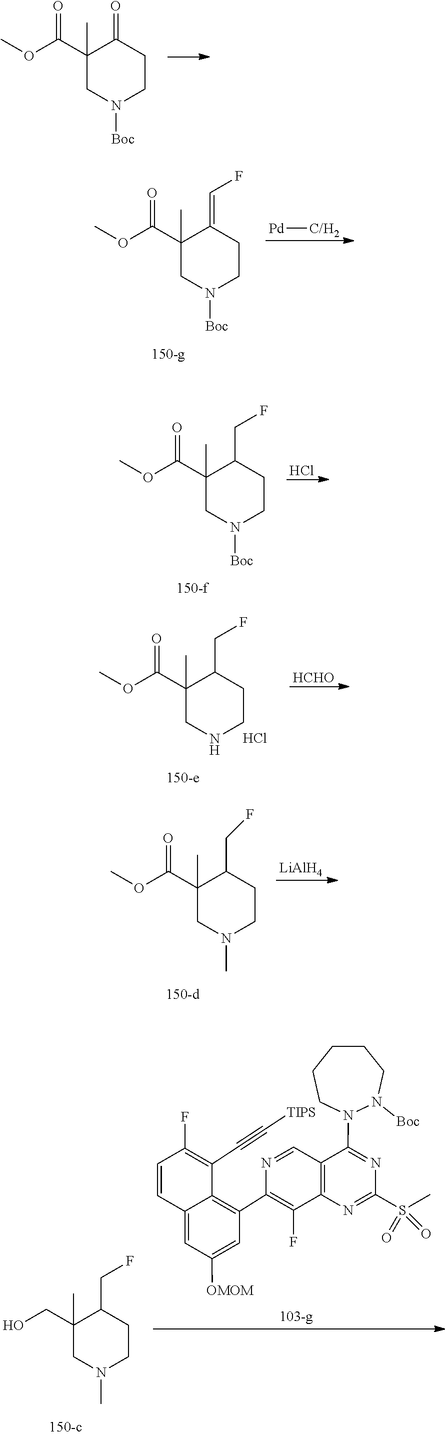

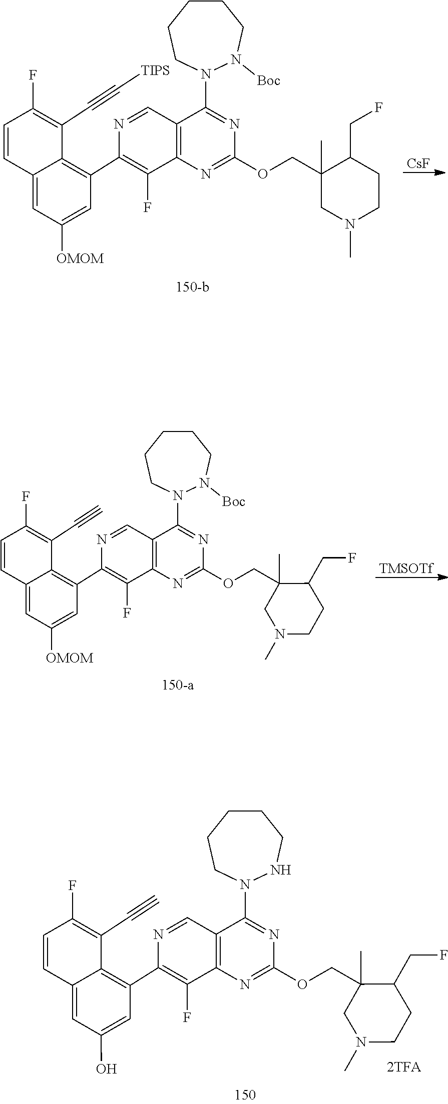

### Synthesis of Compound 150-g

To a reaction flask at room temperature was added (fluoromethyl)triphenylphosphine tetrafluoroborate (2113 mg, 5.53 mmol), THF (10 mL). After degassed and purged with N2, and placed in a dry ice-acetone bath, the mixture was added a solution of potassium tert-butoxide in tetrahydrofuran (1M, 5.53 mg, 5.53 mmol) dropwise. After addition, the reaction mixture was stirred in a dry ice-acetone bath for 1 h, was added 1-tert-butyl-3-methyl-4-oxopiperidine-1,3-dicarboxylic acid-3-methyl ester (1000 mg, 3.69 mmol) dropwise. The reaction mixture was naturally warmed to room temperature and stirred overnight. Water was added to the reaction mixture and the aqueous phase was extracted with EA. The organic phases were combined, washed with water, brine, dried over anhydrous sodium sulfate, concentrated, and purified by column chromatography (mobile phase: PE/EA 10/0 to 8/2) to give compound 150-g (550 mg, 52%). LC-MS (ESI): m/z 288.2 (M+H)⁺.

### Synthesis of compound 150-f

150-g (530 mg, 1.85 mmol), methanol (10 mL), 10% palladium carbon (300 mg) were added to a reaction flask at room temperature. After degassed and purged with hydrogen, the mixture was stirred at room temperature overnight. The reaction mixture was filtered and concentrated to give the crude compound 150-f (540 mg). LC-MS (ESI): m/z 290.2 (M+H)⁺.

### Synthesis of Compound 150-e

To a reaction flask at room temperature were added crude 150-f (540 mg, 1.85 mmol), hydrogen chloride/1,4-dioxane (4M, 5 mL, 20 mmol). After degassed and purged with nitrogen, the mixture was stirred at room temperature for 1 hour. The reaction mixture was concentrated to give the crude compound 150-e (450 mg). LC-MS (ESI): m/z 190.2 (M+H)⁺.

### Synthesis of Compound 150-d

To a reaction flask at room temperature was added crude 150-e (450 mg, 1.85 mmol), methanol (5 mL). After degassed and purged with nitrogen, the mixture was added aqueous formaldehyde solution (³⁷%, 1.5 mL) and stirred for 1 hour at room temperature, then was added sodium cyanoborohydride (232 mg, 3.69 mmol) and stirred at room temperature for 1 hour. The reaction mixture was concentrated, added water and the aqueous phase was extracted with EA. The organic phases were combined, washed with water, brine, dried over anhydrous sodium sulfate, concentrated, and purified by column chromatography (mobile phase: PE/EA 10/0 to 0/10) to give compound 150-d (160 mg, 43%). LC-MS (ESI): m/z 204.3 (M+H)⁺.

### Synthesis of Compound 150-c

150-d (160 mg, 0.79 mmol) and THF (5 mL) were added to a reaction vial at room temperature. After degassed and purged with nitrogen, the mixture was added a solution of lithium aluminum hydride in tetrahydrofuran (4 M, 1.3 mL, 1.3 mmol) dropwise n an ice-water bath, then stirred for 1 h after addition. The reaction mixture was added sodium sulfate decahydrate until no bubbles were produced, then was added a small amount of THF, and dried over anhydrous sodium sulfate. The reaction mixture was filtered through celite, the filter cake was washed with ethyl acetate and the filtrate was concentrated to give compound 150-c (40 mg, 29%). LC-MS (ESI): m/z 176.2 (M+H)⁺.

### Synthesis of compound 150-b

103-g (370 mg, 0.46 mmol) and toluene (5 mL) was added to a reaction vial at room temperature. After degassed and purged with nitrogen, the mixture was added 150-c (40 mg, 0.23 mmol) and sodium tert-butoxide (66 mg, 0.69 mmol) in an ice-water bath. The reaction mixture was stirred in an ice-water bath for 2 hours. The reaction mixture was purified directly by column chromatography (mobile phase: PE/EA 10/0 to 0/10) to afford compound 150-b (40 mg, 19%). LC-MS (ESI): m/z 906.2 (M+H)⁺.

### Synthesis of Compound 150-a

150-b (40 mg, 0.04 mmol) and DMF (1 mL) were added to a reaction vial at room temperature. After degassed and purged with nitrogen, the mixture was added cesium fluoride (34 mg, 0.22 mmol), heated to 50° C., and stirred for 1 hour. The reaction mixture was added water and extracted with EA. The organic phases were combined, washed with water, brine, dried over anhydrous sodium sulfate and concentrated to give the crude compound 150-a (50 mg). LC-MS (ESI): m/z 749.6 (M+H)⁺.

### Synthesis of Compound 150

To a reaction flask at room temperature was added crude 150-a (50 mg, 0.04 mmol) and DCM (2 mL). After degassed and purged with nitrogen, and cooled down to 0° C., the mixture was added trimethylsilyl trifluoromethanesulfonate (0.2 mL) dropwise, then stirred for 1 hour. It was added 10% aqueous sodium bisulfate (1 mL) and stirred for 1 hour. The reaction mixture was adjusted basic with saturated aqueous sodium bicarbonate and the aqueous phase was extracted with DCM. The organic phases were combined, washed with water, brine, dried, concentrated and purified by prep-HPLC to give compound 150 (5 mg, 14%). LC-MS (ESI): m/z 605.2 (M+H)⁺.

### Synthesis of Compound 151

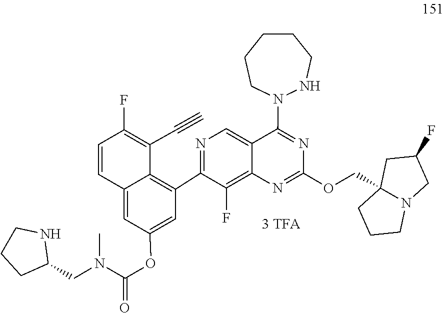 [SMILES: C#Cc1c(F)ccc2cc(OC(=O)N(C)C[C@@H]3CCCN3)cc(-c3ncc4c(N5CCCCCN5)nc(OC[C@@]56CCCN5C[C@H](F)C6)nc4c3F)c12]

Referring to the synthetic route of compound 143, (S)-2-[(methylamino)methyl]-1-pyrrolidinecarboxylic acid tert-butyl ester was used instead of tert-butyl 2-(methylamino)ethyl carbamate to obtain compound 151 by prep-HPLC purification (TFA). LC-MS (ESI): m/z 729.1 (M+H)⁺; ¹H NMR (400 MHz, DMSO-d₆): δ 11.00 (1H, s), 10.32 (1H, s), 9.30 (1H, s), 8.58-8.40 (1H, m), 8.21-8.17 (1H, m), 8.01 (1H, s), 7.78-7.58 (2H, m), 6.01-5.95 (1H, m), 5.65-5.50 (1H, m), 4.63-4.54 (2H, m), 4.20-4.09 (1H, m), 4.06-3.96 (2H, m), 3.85-3.54 (6H, m), 3.22-2.95 (6H, m), 2.57-2.52 (1H, m), 2.48-2.44 (2H, m), 2.20-2.64 (14H, m).

### Synthetic Route of Compound 152

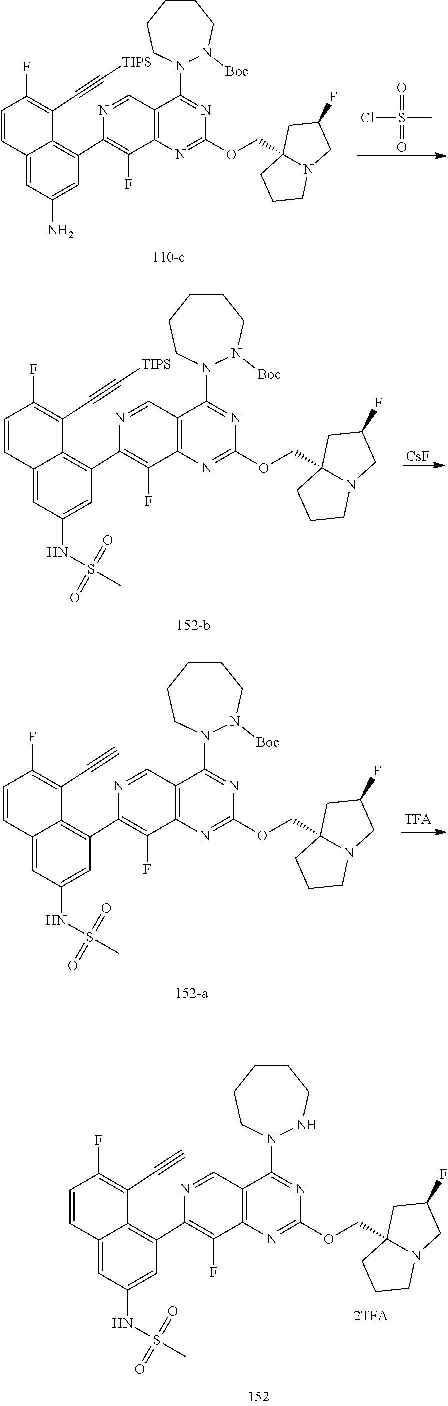

### Synthesis of Compound 152-b

To a solution of 110-c (100 mg, 0.12 mmol) in dichloromethane (10 mL) was added triethylamine (0.082 mL, 0.59 mmol) under ice-water bath conditions, followed by slow addition of methanesulfonyl chloride (0.018 mL, 0.24 mmol). The mixture was warmed to room temperature and stirred for 4 hours. Upon completion, the reaction mixture was removed the organic solvent by concentration at low temperature and reduced pressure. The mixture was purified by column chromatography (mobile phase: methanol/dichloromethane, 0-10%) to give compound 152-b (80 mg, 73%). LC-MS (ESI): m/z 922.5 [M+H]⁺.

### Synthesis of Compound 152-a

To a solution of 152-b (80 mg, 0.087 mmol) in N,N-dimethylformamide (4 mL) was added cesium fluoride (132 mg, 0.87 mmol) at room temperature and the mixture was stirred for 3 h at room temperature. The reaction mixture was poured a large amount of water and the aqueous phase was extracted with ethyl acetate (20 mL*2). The organic phase was washed with brine (15 mL*3), dried over anhydrous sodium sulfate, filtered, concentrated under reduced pressure, and purified by column chromatography (mobile phase: methanol/dichloromethane, 0-10%) to give 152-a (50 mg, 75%). LC-MS (ESI): m/z 766.7 (M+H)⁺.

### Synthesis of Compound 152

Trifluoroacetic acid (3 mL) was added to a solution of 152-a (50 mg, 0.065 mmol) in dichloromethane (10 mL) at room temperature and the mixture was stirred at room temperature for 5 hours. Upon completion, the reaction mixture was concentrated at low temperature under reduced pressure. The resulting crude was purified by Prep-HPLC (acid method TFA) to give 152 (30 mg, 51%). LC-MS (ESI): m/z 666.2 (M+H)⁺; ¹H NMR (DMSO-d₆, 400 MHz): δ 11.95 (1H, s), 10.32 (1H, s), 10.27 (1H, s), 8.13-8.19 (1H, m), 7.93 (1H, s), 7.59 (1H, t, J=8.8 Hz), 7.47 (1H, s), 5.92-6.03 (1H, m), 5.57 (1H, d, J=52.8 Hz), 4.51-4.66 (2H, m), 3.67-4.21 (5H, m), 3.15 (3H, s), 2.98-3.11 (2H, m), 2.53-2.70 (2H, m), 1.60-2.38 (12H, m).

### Synthesis of Compound 153

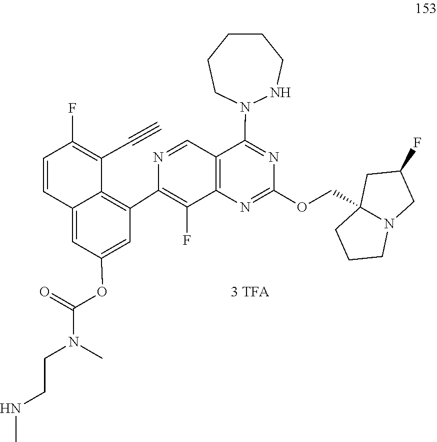 [SMILES: C#Cc1c(F)ccc2cc(OC(=O)N(C)CCNC)cc(-c3ncc4c(N5CCCCCN5)nc(OC[C@@]56CCCN5C[C@H](F)C6)nc4c3F)c12]

Referring to the synthetic route of compound 143, tert-butyl methyl (2-(methylamino)ethyl)carbamate was used instead of tert-butyl 2-(methylamino)ethyl carbamate to give compound 153 by prep-HPLC purification (TFA). LC-MS (ESI): m/z 703.1 (M+H)⁺; ¹H NMR (400 MHz, DMSO): δ 10.99 (1H, s), δ 10.32 (1H, s), δ 8.66 (1H, s), δ 8.55 (1H, s), 8.22-8.18 (1H, dd, J=9.2, 6 Hz), δ 8.03-7.99 (1H, m), 7.68-7.63(H, t, J=9.2 Hz), 7.62-7.59 (1H, m), 5.99 (1H, s), 5.64-5.50 (1H, d, J=52.4 Hz), 4.62-4.52 (2H, m), 4.25-3.52 (7H, m), 3.38-2.90 (8H, m), 2.69-2.42 (6H, m), 2.36-1.87 (7H, m), 1.79-1.57 (4H, m).

### Synthesis of Compound 154

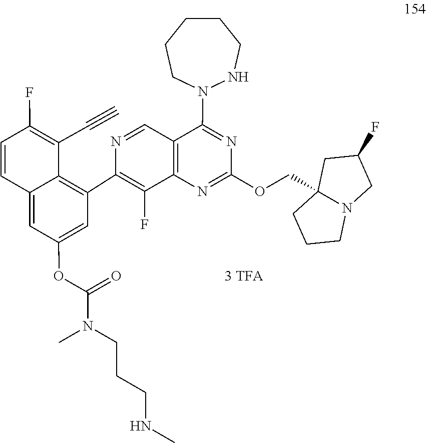 [SMILES: C#Cc1c(F)ccc2cc(OC(=O)N(C)CCCNC)cc(-c3ncc4c(N5CCCCCN5)nc(OC[C@@]56CCCN5C[C@H](F)C6)nc4c3F)c12]

Referring to the synthetic route of compound 143, tert-butyl methyl (3-(methylamino)propyl)carbamate was used instead of tert-butyl 2-(methylamino)ethyl carbamate, to obtain compound 154 by prep-HPLC purification (TFA). LC-MS (ESI): m/z717.2(M+H)⁺.

### Synthetic Route of Compound 155

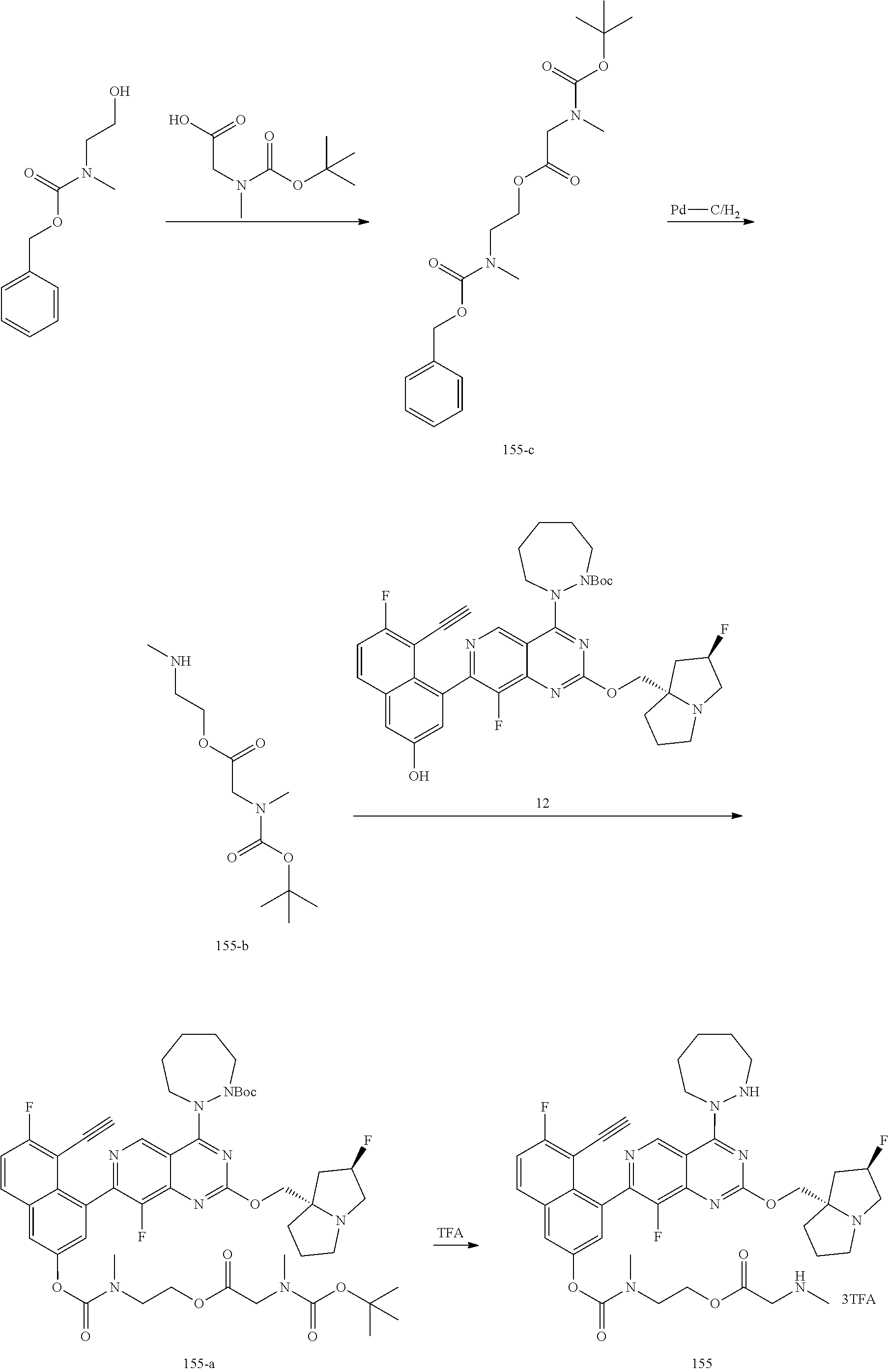

### Synthesis of Compound 155-c

N-methyl-N-Cbz-ethanolamine (812 mg, 3.88 mmol), N-Boc-N-methylglycine (500 mg, 2.46 mmol), DIPEA (1106 mg, 8.56 mmol), HATU (2170 mg, 5.71 mmol) and DMF (5 mL) were separately added to a reaction vial. The reaction mixture was stirred at room temperature under protection of nitrogen for 3 hours. Saturated aqueous sodium bicarbonate (50 mL) was added to the reaction mixture and the aqueous phase was extracted with ethyl acetate (50 mL*3). The organic phase was concentrated and purified by column chromatography (petroleum ether/ethyl acetate=100/0, 3/1) to afford compound 155-c (670 mg, 60%). LC-MS (ESI): m/z=381.2(M+H)⁺.

### Synthesis of Compound 155-b

To a reaction flask were added 155-c (670 mg, 1.76 mmol), palladium carbon (187 mg, 10%) and ethyl acetate (150 mL) at room temperature. After degassed and purged with hydrogen (1000 mL) for three times, the mixture was stirred for 3 hours at room temperature. The reaction mixture was filtered and the organic phase was concentrated to give compound 155-b (419 mg). LC-MS (ESI): m/z=247.7(M+H)⁺.

### Synthesis of Compound 155-a

To a reaction flask were added compound 12 (300 mg, 0.44 mmol), p-nitrophenyl chloroformate (105 mg, 0.52 mmol), DIPEA (169 mg, 1.31 mmol), and dichloromethane (10 mL). The mixture was stirred for 1 h in an ice-water bath under protection of nitrogen, then was added 155-b (129 mg, 0.52 mmol), warmed to room temperature for 12 hours. The reaction mixture was washed with saturated aqueous sodium bisulfate (40 mL) and the organic phase was concentrated to give the crude compound 155-a (420 mg, 100%), which was directly used in the next step. LC-MS (ESI): m/z=961.1(M+H)⁺.

### Synthesis of Compound 155

To a reaction vial were added 155-a (420 mg, 0.44 mmol), dichloromethane (3 mL), trifluoroacetic acid (2 mL), and the mixture was stirred for 3 h at room temperature under protection of nitrogen. The reaction mixture was concentrated and purified by prep-HPLC (TFA) to give compound 155 (46.2 mg) as trifluoroacetate. LC-MS (ESI): m/z=761.4(M+H)⁺.

### Synthetic Route of Compound 156

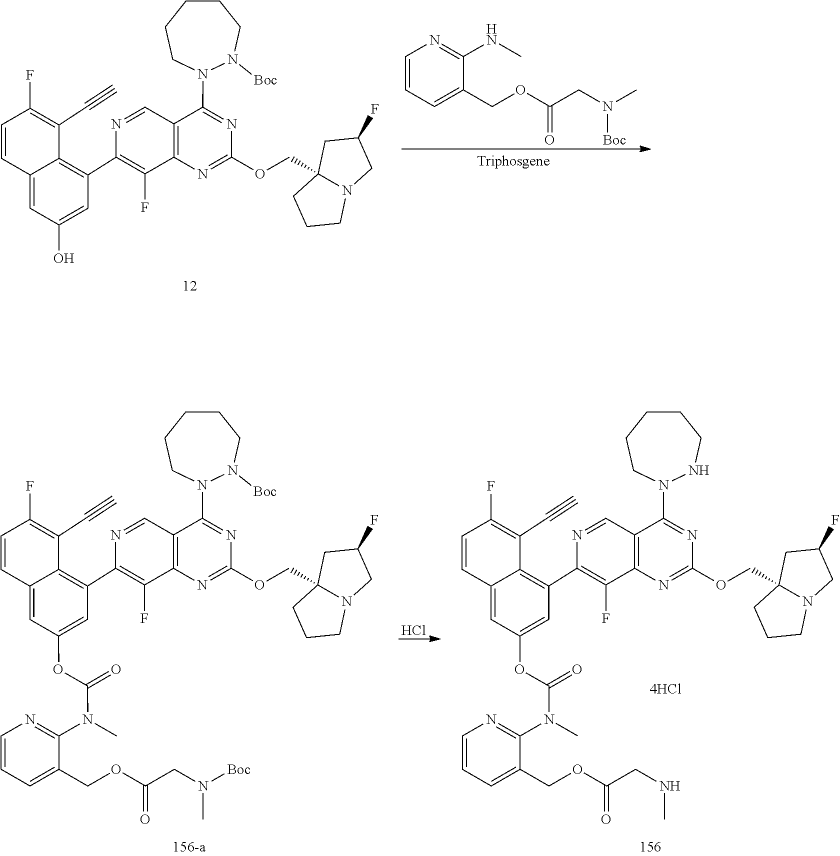

### Synthesis of Compound 156-a

Triphosgene (39 mg, 0.13 mmol) was dissolved in 2.5 mL of anhydrous dichloromethane under protection of nitrogen in an ice-water bath, followed by the dropwise addition of a solution of (2-(methylamino)pyridin-3-yl)methyl 2-((tert-butoxycarbonyl)(methyl)amino)acetate (54 mg, 0.17 mmol) and triethylamine (121 uL, 0.87 mmol) (2.5 mL) in anhydrous dichloromethane. After the dropwise addition, the reaction mixture continued stirring in an ice-water bath for 1 hour. Compound compound 12 (100 mg, 0.15 mmol) and triethylamine (40 uL, 0.29 mmol) were dissolved in 5 mL of anhydrous dichloromethane at room temperature, then was added dropwise to the above reaction mixture in an ice-water bath, followed by the addition of DMAP (53 mg, 0.44 mmol), and the reaction mixture was warmed to room temperature and continued stirring for 1 hr. The reaction mixture was diluted with 100 mL of dichloromethane, washed sequentially with 10% NaHSO₄ (100 mL), saturated sodium bicarbonate solution (100 mL), the organic phase was dried over anhydrous Na₂SO₄, filtered, and the filtrate was concentrated under reduced pressure, and the residue was purified by a flash column chromatography (DCM/MeOH=10:1) to afford compound 156-a (103 mg, 69%). LC-MS (ESI): m/z=1024.5 [M+H]⁺.

### Synthesis of Compound 156

Compound 156-a (103 mg, 0.10 mmol) was dissolved in 5 mL of ethyl acetate at room temperature. Placed in an ice-water bath, the reaction mixture was added hydrogen chloride/ethyl acetate (5 mL, 4 M) dropwise and stirred at room temperature for 1 h, then was added hydrogen chloride/methanol (2 mL, 4 M) and continued stirring for 20 min. The reaction mixture was concentrated under reduced pressure and the residue was dried under vacuum for 1 h. The residue was then suspended in a mixed solvent of acetonitrile and ethyl acetate (1:1, 10 mL) and stirred for 10 min, then was rapidly filtered and the filter cake was washed with a mixed solvent (acetonitrile/ethyl acetate=1:1, 5 mL), the solids were collected and dried under vacuum for 2 h to obtain compound 156 (66 mg). LC-MS (ESI): m/z=824.4[M+H]⁺.

### Synthetic Route of Compound 157

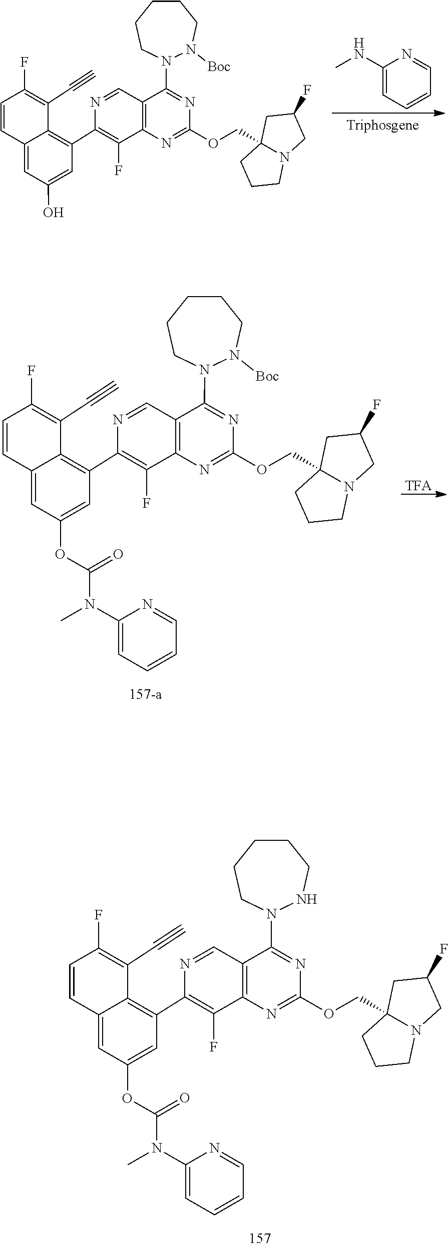

### Synthesis of Compound 157-a

To a solution of 2-methylaminopyridine (45 μL, 0.44 mmol) in DCM (10 mL) in an ice-water bath was added triethylamine (0.20 mL, 1.45 mmol) and triphosgene (129 mg, 0.44 mmol). The reaction mixture was stirred at this temperature for 30 minutes, then was added compound 12 (200 mg, 0.29 mmol) and DMAP (4 mg, 0.029 mmol) and was slowly warmed to room temperature, and stirred at this temperature for 1 hour. Upon completion, water was added to the reaction mixture and the aqueous phase was extracted with dichloromethane (15 mL*2). The combined organic phases were dried over anhydrous sodium sulfate, filtered, concentrated to dryness by rotary evaporation, and the crude was purified by a flash column chromatography to afford 157-a (200 mg, 84%). LC-MS (ESI): m/z 823.9 [M+H]⁺.

### Synthesis of Compound 157

Trifluoroacetic acid (3 mL) was added to a solution of 157-a (200 mg, 0.24 mmol) in dichloromethane (10 mL) at room temperature and the mixture was stirred for 4 h at room temperature. The reaction mixture was concentrated at low temperature, redissolved in dichloromethane, neutralized with saturated sodium bicarbonate solution, partitioned, and the organic phase was concentrated and purified by rapid column chromatography (mobile phase: methanol: dichloromethane=0-10%) to give 157 (120 mg, 68%). LC-MS (ESI): m/z 723.9 [M+H]⁺.

### Synthetic Route of Compound 158

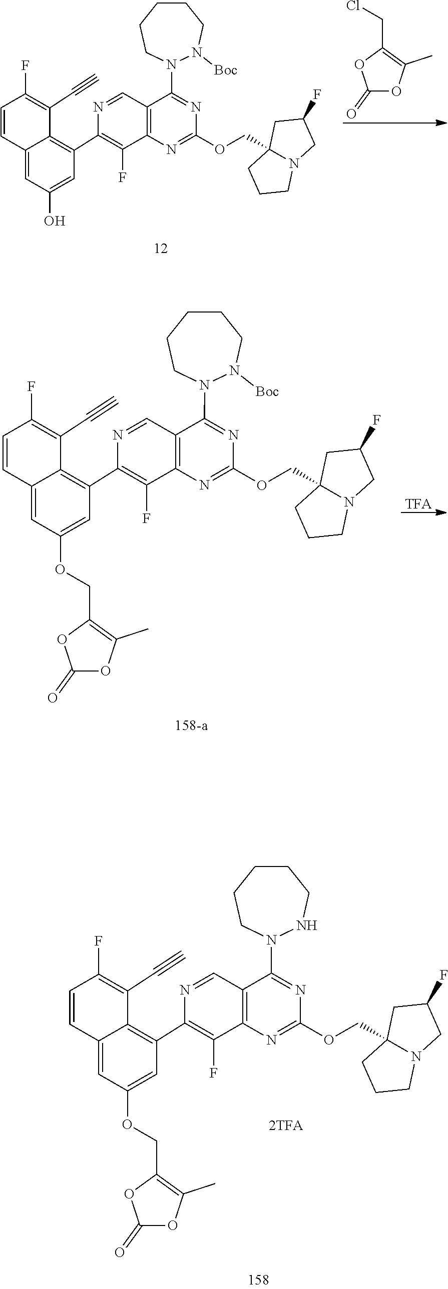

### Synthesis of Compound 158-a

To a solution of compound 12 (300 mg, 0.44 mmol) and potassium carbonate (181 mg, 1.31 mmol) in N,N-dimethylformamide (10 mL) was added 4-chloromethyl-5-methyl-1,3-dioxolen-2-one (129 mg, 0.87 mmol) and potassium iodide (36 mg, 0.22 mmol) at room temperature. The reaction mixture was stirred at this temperature overnight, then was added water and the aqueous phase was extracted with ethyl acetate (30 mL). The organic phase was washed twice with brine, dried over anhydrous sodium sulfate, filtered, concentrated to dryness by rotary evaporation, and the crude was purified by flash column chromatography (mobile phase: ethyl acetate: methanol, 0-10%) to afford 158-a (96 mg, 28%). LC-MS (ESI): m/z 801.8 (M+H)⁺.

### Synthesis of Compound 158

Trifluoroacetic acid (2 mL) was added to a solution of 158-a (96 mg, 0.12 mmol) in dichloromethane (10 mL) at room temperature and the mixture was stirred for 3 h at this temperature. Concentrated to dryness by rotary evaporation and the crude was purified by Prep-HPLC (TFA) to give 158 (27.9 mg, 25%) as TFA salt. LC-MS (ESI): m/z 701.2 (M+H)⁺.

### Synthetic Route of Compound 159

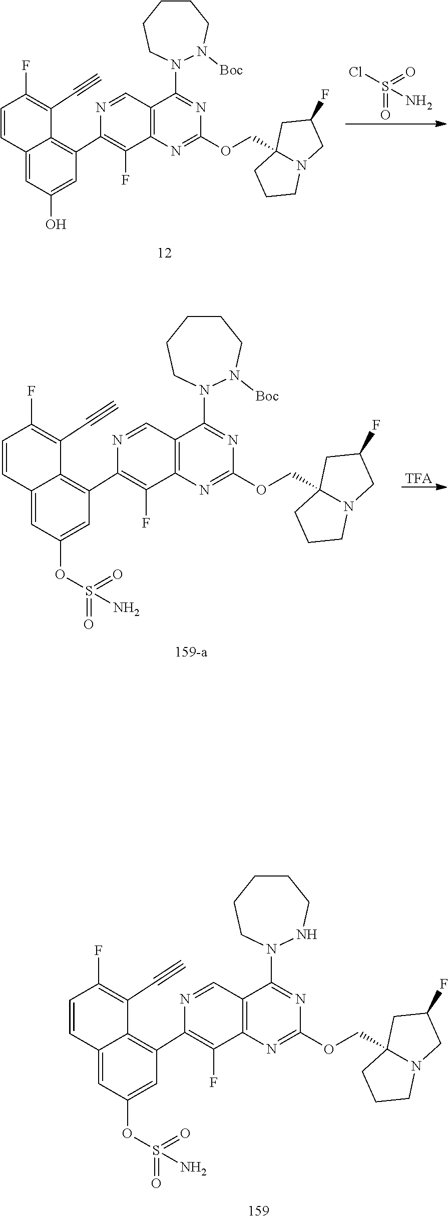

### Synthesis of Compound 159-a

Aminosulfonyl chloride (50 mg, 0.44 mmol) was added to a solution of compound 12 (150 mg, 0.22 mmol) in N,N-dimethylacetamide (8 mL) at room temperature. The reaction mixture was stirred at this temperature for 2 hours, was added water, extracted with ethyl acetate (30 mL), the organic phase was washed twice with saturated brine, dried over anhydrous sodium sulfate, filtered, and concentrated to dryness by rotary evaporation to give 159-a (130 mg, 78%). The product was used directly for the next step without purification. LC-MS (ESI): m/z 768.8 (M+H)⁺.

### Synthesis of Compound 159

Trifluoroacetic acid (2 mL) was added to a solution of 159-a (130 mg, 0.17 mmol) in dichloromethane (10 mL) at room temperature and the mixture was stirred for 2 h at this temperature.

Concentrated to dryness by rotary evaporation and the crude was purified by Prep-HPLC (basic method) to give 159 (15.2 mg, 13%). LC-MS (ESI): m/z 668.3 (M+H)⁺.

### Synthetic Route of Compound 160

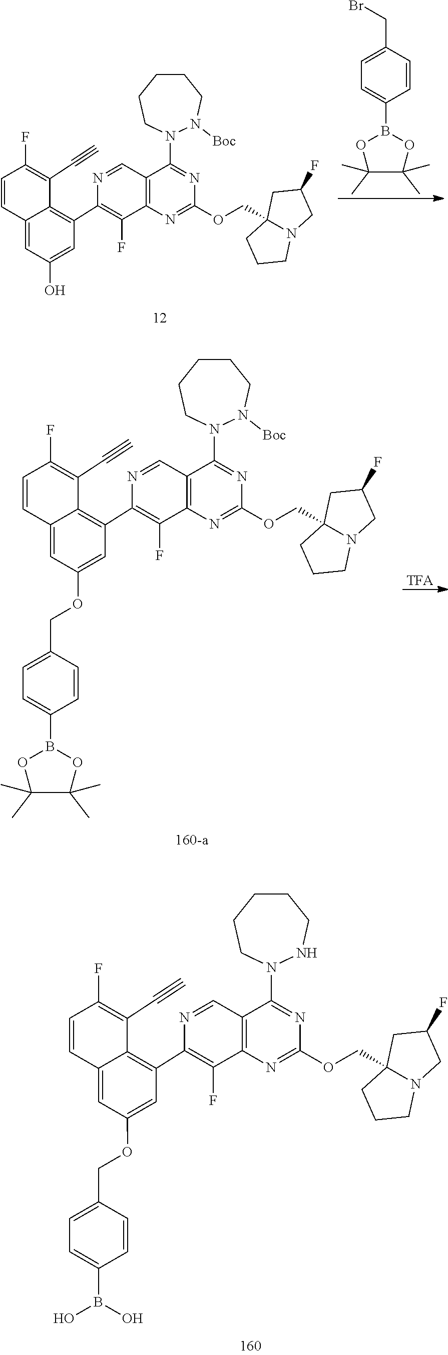

### Synthesis of Compound 160-a

Compound 12 (250 mg, 0.36 mmol), DMF (10 mL), pinacol ester of 4-bromomethylphenylboronic acid (162 mg, 0.55 mmol), potassium carbonate (100 mg, 0.73 mmol) and potassium iodide (6 mg, 0.04 mmol) were added to a reaction flask at room temperature. The reaction mixture was degassed and purged with nitrogen, and stirred for 36 hours at room temperature. Water was added to the reaction mixture and the aqueous phase was extracted with EA. The organic phases were combined, washed with water, brine, dried over anhydrous sodium sulfate, concentrated, and purified by column chromatography (mobile phase: PE/EA 10/0 to 0/10) to give compound 160-a (230 mg, 70%). LC-MS (ESI): m/z 905.5 (M+H)⁺.

### Synthesis of Compound 160

160-a (230 mg, 0.25 mmol), DCM (6 mL) was added to a reaction flask at room temperature. After degassed and purged with nitrogen, the mixture was added TFA (2 mL), then stirred for 2 hours at room temperature. The reaction mixture was concentrated at room temperature and purified by prep-HPLC (basic method) to give compound 160 (23 mg, 13%). LC-MS (ESI): m/z 723.3 (M+H)⁺.

### Synthetic Route of Compound 161

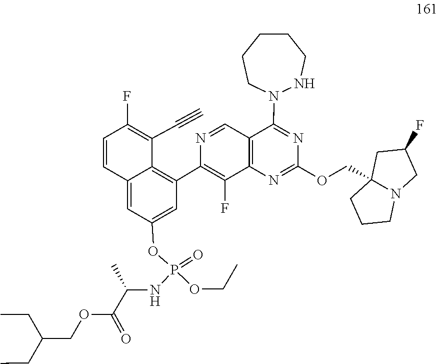 [SMILES: C#Cc1c(F)ccc2cc(OP(=O)(N[C@@H](C)C(=O)OCC(CC)CC)OCC)cc(-c3ncc4c(N5CCCCCN5)nc(OC[C@@]56CCCN5C[C@H](F)C6)nc4c3F)c12]

Referring to the synthetic route of compound 109, (S)-2-aminopropanoic acid (2-ethylbutyl) ester hydrochloride was used instead of L-alanine isopropyl ester hydrochloride to obtain compound 161.

LC-MS (ESI): m/z 852.1 (M+H)⁺; ¹H NMR (400 MHz, DMSO-d₆): δ10.27 (1H, s), 8.22-8.11 (1H, m), δ 8.02-7.90 (1H,m ), 7.70-7.58 (1H, t, J=8.8 Hz), δ 7.57-7.45 (1H,m ), δ 6.19-6.01 (1H,m ), 5.95-5.82 (1H, t, J=5.6 Hz), 5.41-5.18 (1H, d, J=54.8 Hz), 4.25-3.76 (10H, m), 3.22-2.79 (6H, m), 2.23-1.55 (11H, m), 1.34-1.06 (12H, m), 0.83-0.65 (6H, m).

### Synthetic Route of Compound 162

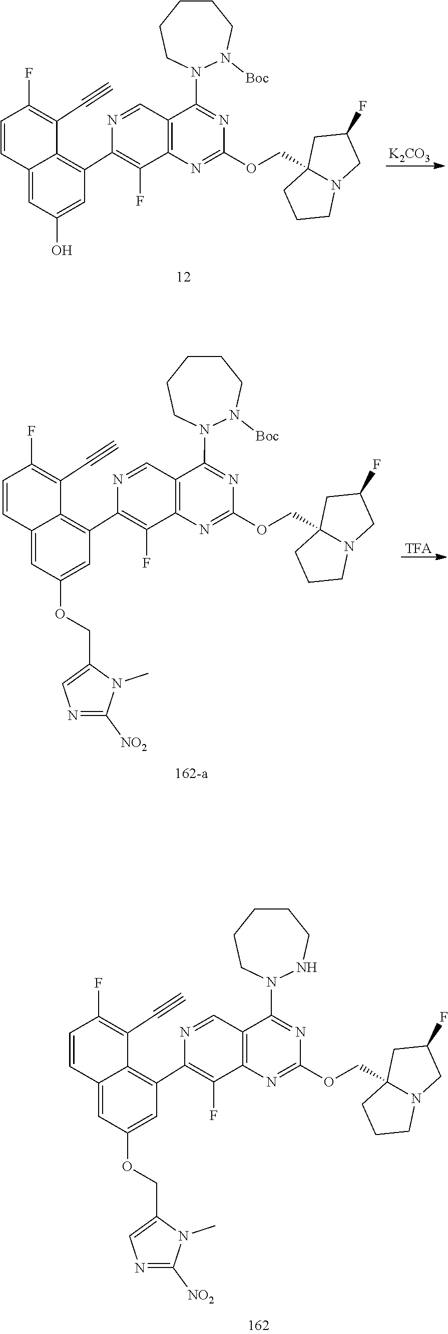

### Synthesis of Compound 162-a

To a solution of compound 12 (250 mg, 0.36 mmol) in DMF (10 mL) was added potassium carbonate (150 mg, 1.09 mmol) and potassium iodide (30 mg, 0.18 mmol) at room temperature. The reaction mixture was stirred at this temperature overnight. Upon completion, the reaction mixture was added water, extracted with ethyl acetate, the organic phase was washed twice with brine, dried over anhydrous sodium sulfate, filtered, concentrated to dryness by rotary evaporation, and the crude was purified by rapid column chromatography (mobile phase: methanol/dichloromethane, 0-10%) to give 162-a (200 mg, 67%). LC-MS (ESI): m/z 828.9 [M+H]⁺.

### Synthesis of Compound 162

Trifluoroacetic acid (3 mL) was added to a solution of 162-a (200 mg, 0.24 mmol) in dichloromethane (10 mL) at room temperature and the mixture was stirred at room temperature for 4 h. The reaction mixture was concentrated at low temperature, redissolved in dichloromethane, neutralized with saturated sodium bicarbonate solution, partitioned, and the organic phase was concentrated and purified by flash column chromatography (mobile phase: methanol: dichloromethane=0-10%) to give 162 (130 mg, 74%). LC-MS (ESI): m/z 728.8 [M+H]⁺; ¹H NMR (DMSO-d₆, 400 MHz): δ 10.26 (1H, s), 8.12-8.09 (1H, m), 7.85 (1H, d, J=2.0 Hz), 7.58 (1H, t, J=8.8 Hz), 7.44 (1H, s), 7.41 (1H, s), 5.88 (1H, t, J=6.0 Hz), 5.45 (2H, s), 5.30 (1H, d, J=54.4 Hz), 3.92-4.22 (5H, m), 4.30 (3H, s), 2.97-3.23 (5H, m), 2.80-2.91 (1H, m), 1.57-2.22 (12H, m).

### Effect Example 1 Proliferation Inhibition of Compounds on RAS Cell Lines Measured by CTG

NCI-H358 is a human non-small cell lung cancer cell line with KRAS G12C mutation; AGS is a gastric cancer cell line with KRAS G12D mutation; SW620 is a colon cancer cell with KRAS G12V mutation; NCI-H460 is a non-small cell lung cancer cell with KRAS Q61H mutation; MKN1 is a wild-type gastric cancer cell with KRAS-amplified; LS513 is a colon cancer cell with KRAS G12D mutation; LoVo is a colon cancer cell with KRAS G13D mutation; NCI-H441 is a lung cancer cell with KRAS G12V mutation, and Capan-2 is a pancreatic cancer cell with KRAS G12V mutation.

The proliferation inhibitory effect of the compounds on various mutations was evaluated by determining the proliferation inhibitory activity of the compounds on these cell lines.

The assay was conducted on either 384-well plates or 96-well plates. The procedures are as follows:

The cell suspensions were added to 384- or 96-well plates (384-well plate: 50 μL; 96-well plate: 100 μL) except for peripheral wells. The plates were placed in a carbon dioxide incubator overnight. NCI-H358, AGS, NCI-H460, MKN-1, LS513, LoVo, and NCI-H441 were placed in a 5%, 370 carbon dioxide incubator overnight, and SW620 was placed in a 0%, 370 carbon dioxide incubator overnight. The HPD300 micro-dosing instrument was used to add the corresponding concentration of compound to each well (9-10 concentration gradients of compound were obtained by 3-fold dilution). Cell plates were incubated in a carbon dioxide incubator for 5-6 days. NCI-H358, AGS, NCI-H460 and MKN-1 were incubated at 5%, 37° incubator and SW620 was incubated at 0%, 370 incubator. On the day of plate measurement, the 384- or 96-well plates were first equilibrated at room temperature for 10-30 min, then CellTiter Glo reagent (384-well plate: 25 μL; 96-well plate: 100 μL) was added to the 384- or 96-well plate, shaken for 10 min away from light, and incubated for 10 min. The plates were placed into Victor NIVO or ENVIDION reader plates, and XLFit was utilized to plot the pharmacodynamic inhibition curve and calculate the IC50 value. The activity results for representative compounds are shown in Tables 1, 2 and 3 below. “IC₅₀>10 μM” is denoted by “*”, “10 μM≥IC₅₀≥1 μM” is denoted by “**”, and “1 μM≥IC₅₀>100 nM” is denoted by “***”, “IC₅₀<100 nM” is denoted by “****”

TABLE 1
Proliferation inhibitory activity of representative compounds of the present disclosure on RAS cells

| | | | | IC₅₀ | | | | IC₅₀ |
|---|---|---|---|---|---|---|---|---|
| Compound | IC₅₀ | IC₅₀ | IC₅₀ | (NCI- | IC₅₀ | IC₅₀ | IC₅₀ | (NCI- |
| No. | (AGS ^{a}) | (H358 ^{b}) | (SW620 ^{c}) | H460 ^{d}) | (MKN1 ^{e}) | (LS513^{f}) | (LoVo^{g}) | H441^{h}) |
| 1-1 | ** | — | — | — | — | — | — | — |
| 1-2 | *** | — | — | — | — | — | — | — |
| 2-1 | **** | **** | **** | ** | **** | **** | **** | *** |
| 2-2 | *** | *** | *** | — | — | — | — | — |
| 3 | **** | **** | **** | *** | **** | — | — | — |
| 4 | **** | **** | **** | *** | **** | **** | **** | **** |
| | | | | | | 0.0036 uM | 0.0078 uM | 0.0036 uM |
| 6 | *** | *** | *** | ** | ** | — | — | — |
| 7 | **** | **** | **** | ** | *** | — | — | — |
| 8 | **** | **** | **** | *** | **** | — | — | — |
| 9 | — | — | — | — | — | **** | **** | **** |
| 10 | — | — | — | — | — | **** | *** | *** |
| 11 | — | — | — | — | — | **** | *** | *** |
| 12 | — | — | — | — | — | **** | — | — |
| 13 | — | — | — | — | — | **** | — | — |
| 14 | — | — | — | — | — | *** | — | — |
| 16 | — | — | — | — | — | **** | — | — |
| | | | | | | 0.0118 uM | | |
| 21 | — | — | — | — | — | **** | — | — |
| 22 | — | — | — | — | — | **** | — | — |
| Comparative | — | — | — | — | — | **** | **** | **** |
| compound 1′ | | | | | | 0.0063 uM | 0.0297 uM | 0.0373 uM |

Note:
^{a} incubated for 5 days;
^{b, c, d, e, f, g, h}incubated for 6 days.

TABLE 2
Proliferation inhibitory activity of representative
compounds of the present disclosure on RAS cells

| | IC₅₀ | IC₅₀ | IC₅₀ | IC₅₀ | IC₅₀ |
|---|---|---|---|---|---|
| Compound No. | (SW620^{c}) | (LS513^{f}) | (NCI-H441^{h}) | (Capan-2ⁱ) | (GP2D^{j}) |
| 23 | — | **** | **** | — | — |
| 24 | — | **** | *** | — | — |
| 25 | — | **** | — | *** | — |
| 26 | — | *** | — | ** | — |
| 27 | — | *** | — | ** | — |
| 28 | — | **** | — | *** | — |
| 29 | — | **** | — | **** | — |
| 30 | **** | **** | — | — | — |
| 32 | — | **** | — | — | — |
| 33 | — | **** | — | *** | — |
| 34 | **** | **** | — | — | — |
| 35 | — | **** | — | *** | — |
| 36 | — | **** | — | **** | — |
| 37 | **** | **** | — | — | — |
| 38 | ** | — | — | — | — |
| 39 | ** | — | — | — | — |
| 40 | — | ** | — | — | — |
| 41 | ** | — | — | — | — |
| 42 | — | *** | — | — | — |
| 43 | — | **** | — | — | — |
| 44 | — | **** | — | — | — |
| 46 | — | **** | — | — | — |
| 47 | — | **** | — | — | — |
| 48 | — | **** | — | — | — |
| 49 | **** | **** | — | — | — |
| 50 | *** | *** | — | — | — |
| 51 | — | **** | — | — | — |
| 15 | — | *** | *** | — | — |
| 17 | — | **** | *** | — | — |
| 18 | — | ** | ** | — | — |
| 19 | — | **** | ** | — | — |
| 20 | — | *** | — | — | — |
| Comparative compound 2′ | — | **** | — | — | — |
| | — | 0.0396 uM | — | — | — |
| 52 | — | *** | — | — | — |
| 53 | — | *** | — | — | — |
| 54 | — | — | — | — | *** |
| 55-2 | — | *** | — | — | — |
| 56 | — | **** | — | — | — |
| 58 | — | **** | — | — | **** |
| 60 | — | **** | — | — | **** |
| 64 | — | **** | — | — | **** |
| 66 | — | — | — | — | **** |
| 68 | — | **** | — | — | **** |
| 69 | — | **** | — | — | **** |
| 72 | — | **** | — | — | **** |
| 76 | — | — | — | — | *** |
| 81 | — | **** | — | — | *** |
| 82 | — | **** | — | — | **** |
| 85 | — | *** | — | — | *** |
| 86 | — | **** | — | — | **** |

Note:
^{c, f, h, i, j,}incubated for 6 days.

TABLE 3
Proliferation inhibitory activity of representative
compounds of the present disclosure on RAS cells

| Compound | IC₅₀ | IC₅₀ | Compound | IC₅₀ | IC₅₀ |
|---|---|---|---|---|---|
| No. | (LS513^{k}) | (GP2D^{l}) | No. | (LS513^{k}) | (GP2D^{l}) |
| 103 | **** | **** | 105 | **** | **** |
| 106 | **** | **** | 107 | *** | **** |
| 110 | **** | **** | 116 | *** | **** |
| 117 | *** | *** | 118 | *** | *** |
| 121 | *** | *** | 122 | *** | **** |
| 123 | **** | **** | 125 | *** | *** |
| 130 | — | *** | 131 | *** | *** |
| 134 | *** | ** | 137 | *** | *** |
| 138 | — | *** | 73 | *** | *** |
| 74 | **** | **** | 89 | — | *** |
| 90 | **** | **** | 91 | **** | **** |
| 92 | **** | **** | 93 | **** | **** |
| 94 | **** | **** | 95 | **** | **** |
| 96 | **** | **** | 97 | *** | *** |
| 98 | **** | **** | 99 | **** | **** |
| 101 | **** | **** | 102 | **** | **** |
| 108 | — | **** | 111 | — | **** |
| 126 | **** | **** | 133 | **** | **** |
| 145 | *** | *** | 148 | — | **** |
| 149 | **** | **** | 150 | **** | **** |
| 151 | **** | **** | 153 | **** | **** |

Note:
^{k, l,}was inclubated for 6 days.

### Effectiveness Example 2 Pharmnacokinetic Study in Mice after Intravenous and Oral Administration of the Drug

### Experimental Procedure

After intravenous and oral administration in male C57BL/6J mice (6-8 weeks), plasma samples were collected through the orbital venous plexus at 8 time points (5 min, 15 min, 30 min, 1 h, 2 h, 4 h, 7 h, 24 h), and the collected blood samples were transferred to microcentrifuge tubes containing EDTA-K2 anticoagulant, and centrifuged at 4° C., 4,000 g. After 5 min, the supernatant was taken and stored in a refrigerator at −75° C. ±15° C. LC-MS/MS was used to detect the concentrations of the compounds in the plasma at different time points, and the relevant pharmacokinetic parameters PGP-241,T1 were calculated using WinNonlin software. The experiment result is shown in Tables 4.

TABLE 4

| | | |
|---|---|---|
| | Comparative | |
| Compound | compound 1′ | Compound 4 |

PK data for intravenous administration

| | | |
|---|---|---|
| Dose (mg/kg) | 5 | 5 |
| t_{1/2} (h) | 1.02 | 2.63 |
| C₀ (ng/mL) | 3422 | 96074 |
| AUCₗₐₛₜ (h*ng/mL) | 1938 | 24264 |
| AUC_{inf} (h*ng/mL) | 1943 | 24271 |
| CL (mL/min/kg) | 42.9 | 3.43 |
| Vₛₛ (L/kg) | 1.77 | 0.205 |

PK data for oral administration

| | | |
|---|---|---|
| Dose (mg/kg) | 30 | 30 |
| t_{1/2} (h) | 1.68 | 1.89 |
| Tₘₐₓ (h) | 0.500 | 4.00 |
| Cₘₐₓ (ng/mL) | 790 | 2292 |
| AUCₗₐₛₜ (h*ng/mL) | 1085 | 10428 |
| AUC_{inf} (h*ng/mL) | 1122 | 10430 |

Note:
Intravenous solution is 5% DMSO + 10% Solutol HS15 + 85% (20% HP-β-CD in water); oral administration solution is 0.1% Tween 80 + 0.5% MC in water

Although specific embodiments of the present disclosure have been described above, it will be appreciated by those skilled in the art that these embodiments are merely illustrative and that many changes or modifications can be made to these embodiments without departing from the principles and spirit of the present disclosure. The scope of protection of the present disclosure is therefore defined by the appended claims.

1. A nitrogen-containing heterocyclic compound of formula I, a pharmaceutically acceptable salt thereof, a prodrug thereof, a stereoisomer thereof, a tautomer thereof or an isotopically labeled compound thereof: 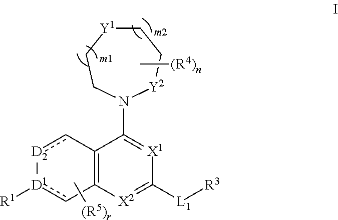
wherein [symbol] represents a single or double bond; 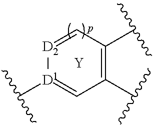
is phenyl, “5- to 7-membered heterocycloalkenyl containing 1-3 heteroatoms independently selected from O, S and N”, “5- to 7-membered heteroaryl containing 1-3 heteroatoms independently selected from O, S and N” or 5- to 7-membered cycloalkenyl; wherein D¹ is C, CH or N; D₂ is 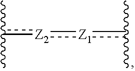
wherein Z₁ and Z₂ are independently a bond, CH, CH₂, O, S, N or NH;
r is 0, 1, 2, 3 or 4;
R⁵ is independently deuterium, halogen, C1-6 alkyl, C1-6 alkyl substituted with one or more halogen, C3-10 cycloalkyl, hydroxyl, C1-6 alkyl-O—, C1-6 alkyl-S—, amino, C1-6 alkyl-NH—, C2-6 alkenyl, nitro or C3-10 cycloalkyl-O—;
X¹ and X² are independently CR^{b} or N, and X¹ and X² are not both CR^{b}; 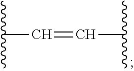 [SMILES: *C=C*]
Y¹ is O, S, NH, CH₂ or 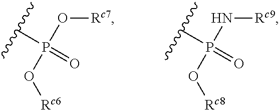
Y² is NR², 0 or S;
m1, m2 and n are independently 0, 1, 2, 3 or 4;
R^{b} is hydrogen, halogen or cyano;
R¹ is C₆₋₂₀ aryl, “5- to 12-membered heteroaryl containing 1-4 heteroatoms independently selected from O, S and N”, C₆₋₂₀ aryl substituted with one or more R^{1′}— or “5- to 12-membered heteroaryl containing 1-4 heteroatoms independently selected from O, S and N” substituted with one or more R¹⁻²; provided that when multiple substituents are present, the substituents are the same or different;
R^{1′-1} and R¹⁻² are independently deuterium, halogen, —OR^{c}, cyano, azide, —C(═O)R¹¹, —NR¹²R¹³, —C(═O)OR¹⁴, —C(═O)NR¹⁵R¹⁶, C₁₋₆ alkyl, C₂₋₆ alkenyl, C₂₋₆ alkynyl, C₃₋₁₀ cycloalkyl, —B(OR^{x}) (OR^{y}), “4- to 7-membered heterocycloalkyl containing 1 or 2 heteroatoms independently selected from O, S and N”, C₆₋₂₀ aryl, “5- to 7-membered heteroaryl containing 1 or 2 heteroatoms independently selected from O, S, and N”, C₁₋₆ alkyl substituted by one or more R¹⁻¹⁻¹, C₁₋₆ alkyl-O— substituted by one or more R¹⁻¹⁻², C₃₋₁₀ cycloalkyl substituted by one or more R¹⁻¹⁻³, “4- to 7-membered heterocyclic cycloalkyl containing 1 or 2 heteroatoms independently selected from O, S, and N” substituted by one or more R¹⁻¹⁻⁴, C₆₋₂₀ aryl substituted with one or more R^{1′-5}, or “5- to 7-membered heteroaryl containing one or two heteroatoms independently selected from O, S, and N” substituted with one or more R¹⁻¹⁻⁶; provided that when multiple substituents are present, the substituents are the same or different; or, when the number of R¹⁻¹ or R¹⁻² is more than one, two optional R¹⁻¹ or R¹⁻² are connected, together with the atoms on the ring to which they are attached, independently form 3- to 8-membered cyclic olefin;
R², R^{c}, R¹² and R¹³ are independently hydrogen, C₁₋₆ alkyl, —C(═O)R^{c1}, —C(═O)OR^{c2}, —C(═O)NR^{c3}R^{c4}, —SO₂R^{c5},
or C₁₋₆ alkyl substituted with one or more R^{c0};
R^{c1}, R^{c2}, R^{c3}, R^{c4}, R^{c5}, R^{c6}, R^{c7}, R^{c8} and R^{c9} are independently hydrogen, C₁₋₂₀ alkyl, C₃₋₁₀ cycloalkyl, “4- to 7-membered heterocycloalkyl containing 1 or 2 heteroatoms independently selected from O, S and N”, C₆₋₂₀ aryl, “5- to 7-membered heteroaryl containing 1 or 2 heteroatoms independently selected from O, S and N”, C₁₋₂₀ alkyl substituted with one or more R⁴⁻¹⁻¹, C₃₋₁₀ cycloalkyl substituted with one or more R⁴⁻¹⁻², “4- to 7-membered heterocycloalkyl containing 1 or 2 heteroatoms independently selected from O, S and N” substituted with one or more R⁴⁻¹⁻³, C₆₋₂₀ aryl substituted with one or more R⁴⁻¹⁻⁴, “5- to 7-membered heteroaryl containing 1 or 2 heteroatoms independently selected from O, S and N”, “5- to 7-membered heteroaryl containing 1 or 2 heteroatoms independently selected from O, S and N” substituted with one or more R⁴⁻¹⁻⁵, —NR^{c51}R^{c52}, —(C₁-₆ alkylene-O)ₚ— C₁-6 alkyl, 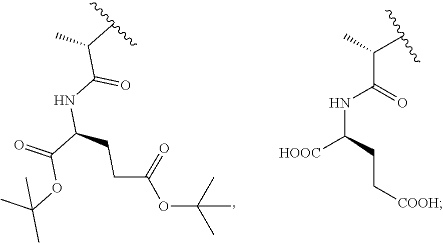 [SMILES: *[C@@H](C)C(=O)N[C@@H](CCC(=O)OC(C)(C)C)C(=O)OC(C)(C)C ; *[C@@H](C)C(=O)N[C@@H](CCC(=O)O)C(=O)O]
p is 1, 2,3 or 4;
R¹⁻¹⁻¹, R¹⁻¹⁻², R¹⁻¹⁻³, R¹⁻¹⁻⁴, R¹⁻¹⁻⁵, R¹⁻¹⁻⁶, R⁴⁻¹⁻¹, R⁴⁻¹⁻², R⁴⁻¹⁻³, R⁴⁻¹⁻⁴ and R⁴⁻¹⁻⁵ are independently cyano, halogen, hydroxyl, nitro, C₁₋₆ alkyl-O—, C₁₋₆ alkyl, C₁₋₆ alkyl substituted with one or more R³¹, C₃₋₁₀ cycloalkyl, “4- to 7-membered heterocycloalkyl containing 1 or 2 heteroatoms independently selected from O, S, and N”, C₆₋₂₀ aryl, “5- to 7-membered heteroaryl containing 1 or 2 heteroatoms independently selected from O, S, and N”, —B(OR^{x}) (OR^{y}), —C(═O)R²1, —NR²²R²³, —C(═O)OR²⁴, —OC(═O)R³², —C(═O)NR²⁵R²⁶, —COOH, —C(═O)OR²⁷ or —OC(═O)OR²⁸;
R^{c10} is —OC₁₋₆ alkyl, hydroxyl, 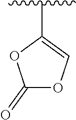 [SMILES: *c1coc(=O)o1]
substituted with C₁₋₆ alkyl, C₆₋₂₀ aryl substituted with one or more —B(OR^{x})(OR^{y}), “5- to 7-membered heteroaryl containing 1 or 2 heteroatoms independently selected from O, S, and N” substituted with one or more R^{c10-1}, 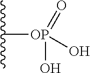
or —OC(═O)R²⁹; R^{c10-1} is independently C₁₋₆ alkyl or nitro;
or, optional R² and R^{b}, optional R² and R⁵ together with the atoms to which they are attached, independently form “5- to 12-membered heterocyclic alkenyl containing 2 to 4 heteroatoms, two of which are N, and the other heteroatoms independently selected from O, S, and N”, or “5- to 12-membered heterocyclic alkenyl containing 2 to 4 heteroatoms, two of which are N, and the other heteroatoms independently selected from O, S, and N,” substituted with one or more R^{a};
R^{x} and R^{y} are independently hydrogen, C₁₋₆ alkyl, or R^{x} and R^{y} each with the attached oxygen atom form five-membered ring substituted with one or more C₁₋₆ alkyl;
R⁴ is independently deuterium, halogen, cyano, hydroxyl, C₁₋₆ alkyl, C₁₋₆ alkyl substituted with one or more R⁴⁻¹ C₂₋₆ alkenyl, C₂₋₆ alkynyl, C₃₋₁₀ cycloalkyl, C₁₋₆ alkyl-O—, —O—C₁₋₆ alkyl substituted with one or more R⁴⁻², —S—C₁₋₆ alkyl, —S—C₁₋₆ alkyl substituted with one or more R⁴⁻³, O═, 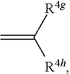
—O—C₃₋₁₀ cycloalkyl, —C(═O)R^{4a}, —NR^{4b1}R^{4b2}, —C(═O)OR^{4c} or —C(═O)NR^{4d1}R^{4d2}; or, when n is 2, 3 or 4, two optional R⁴ are connected, together with the atoms to which they are attached form 3- to 8-membered carbocyclic ring, 3- to 8-membered carbocyclic ring substituted with one or more R⁴⁻⁴, “4- to 8-membered heterocyclic ring containing 1 to 3 heteroatoms independently selected from O, S, and N”, or, “4- to 8-membered heterocyclic ring containing 1 to 3 heteroatoms independently selected from O, S, and N” substituted with one or more R⁴⁻⁵;
R⁴⁻¹, R⁴⁻², R⁴⁻³, R⁴⁻⁴, R⁴⁻⁵ and R^{a} are independently halogen, C₁₋₆ alkyl, C₂₋₆ alkenyl, cyano, hydroxyl, C₁₋₆ alkyl-O—, —NR⁴ⁱR^{4j}, —C(═O)OR^{4e} or —C(═O)NR^{4f1}R^{4f2}; or, R⁴⁻⁵ is O═;
R^{4a}, R^{4b1}, R^{4b2}, R^{4c}, R^{4d1}, R^{4d2}, R⁴, R^{4f1}, R^{4f2}, R⁴ⁱ and R^{4j} are independently hydrogen, C₁₋₆ alkyl or C₂₋₆ alkenyl;
L₁ is a bond, C₁₋₆ alkylene, —C(═O)—, —O(R^{L-1})ₙ₁—, —S(R^{L-2})ₙ₂- or —NR^{L-3} (R^{L-4})ₙ₃—; R^{L-1}, R^{L-2} and R^{L-4} are independently C₁₋₆ alkylene or C₁₋₆ alkylene substituted with one or more deuterium; R^{L-3} is hydrogen or C₁₋₆ alkyl; n1, n2 and n3 are independently 0 or 1;
R³ is C₃₋₁₂ cycloalkyl, C₃₋₁₂ cycloalkyl substituted with one or more R³⁻¹, “4- to 12-membered heterocycloalkyl containing 1-3 heteroatoms independently selected from O, S and N”, “4- to 12-membered heterocycloalkyl containing 1-3 heteroatoms independently selected from O, S and N” substituted with one or more R³⁻², C₁₋₆ alkyl, C₁₋₆ alkyl substituted with one or more R³⁻³, C₂₋₆ alkenyl substituted with one or more R³⁻⁴, —OR^{d}, —SR^{d1}, —NR^{e1}R^{e2} or —C(═O)NR^{e3}R^{e4}; provided that when multiple substituents are present, the substituents are the same or different;
R³⁻¹, R³⁻², R³⁻³ and R³⁻⁴ are independently C₁₋₆ alkyl, C₁₋₆ alkyl substituted with one or more R³⁻¹⁻¹, C₆₋₂₀ aryl substituted with one or more R³⁻¹⁻⁵, hydroxyl, azide, Ar, C₁₋₆ alkyl-O—, —CH₂—O—Ar, halogen, O═, —NR^{e5}R^{e6}, —C(═O)NR^{e7}R^{e8} or 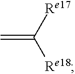
Ar is “5-12-membered heteroaryl containing 1-3 heteroatoms independently selected from O, N and S” substituted with one or more R³⁻¹⁻⁴.
or, R³⁻¹ and R³⁻² are independently -L₂-M; L₂ is -L₂₁-L₂₂-L₂₃-L₂₄-L₂₅-L₂₆; L₂₁, L₂₃ and L₂₅ are independently a bond, O, S, NH, C(O)NH or C₁₋₆ alkylene; L₂₂ and L₂₄ are independently a bond, C₃₋₁₀ cycloalkyl, “4- to 7-membered heterocycloalkyl containing 1 or 2 heteroatoms independently selected from O, S and N”, C₆₋₂₀ aryl, or, “5- to 7-membered heteroaryl containing 1 or 2 heteroatoms independently selected from O, S, and N”; L₂₆ is a bond, O, S, NH or C(O)NH;
M is, 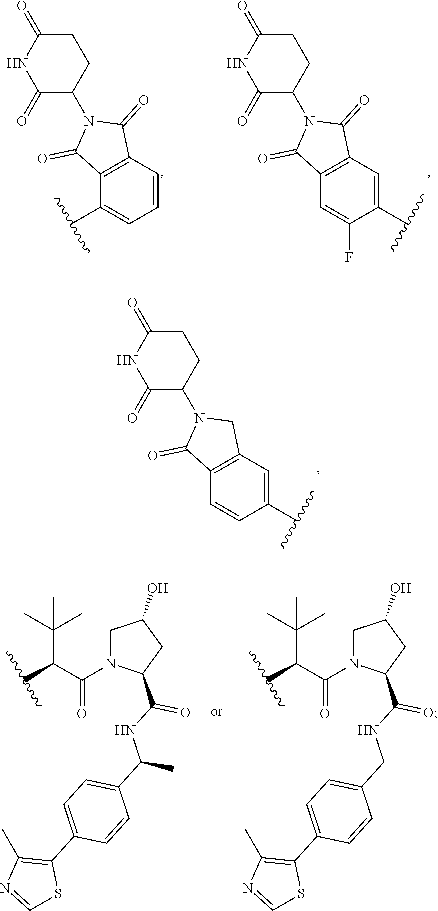 [SMILES: *c1cccc2c1C(=O)N(C1CCC(=O)NC1=O)C2=O ; *c1cc2c(cc1F)C(=O)N(C1CCC(=O)NC1=O)C2=O ; *c1ccc2c(c1)CN(C1CCC(=O)NC1=O)C2=O ; *[C@H](C(=O)N1C[C@H](O)C[C@H]1C(=O)N[C@@H](C)c1ccc(-c2scnc2C)cc1)C(C)(C)C ; *[C@H](C(=O)N1C[C@H](O)C[C@H]1C(=O)NCc1ccc(-c2scnc2C)cc1)C(C)(C)C]
R^{4g}R^{4h}, R^{e17} and R^{e18} are independently hydrogen, halogen, C₁₋₆ alkyl, C₁₋₆ alkyl substituted with one or more halogen, cyano, —C(═O)R^{e171}, —C(═O)OR^{e172} or —C(═O)NR^{e173}R^{e174}; or, R^{4g} and R^{4h}, R^{e17} and R^{e18}, together with the atom to which they are attached form 3- to 8-membered carbocyclic ring, or “4- to 8-membered heterocyclic ring containing 1 to 3 heteroatoms independently selected from O, S, and N”;
R^{d}, Rad, R^{e1}, R^{e2}, R^{e3} and R^{e4} are independently hydrogen, C₁₋₆ alkyl, C₃₋₁₀ cycloalkyl, “4- to 10-membered heterocycloalkyl containing 1-3 heteroatoms independently selected from O, S and N”, or, C₁₋₆ alkyl substituted with one or more R³⁻¹⁻²
R³⁻¹⁻¹, R³⁻¹⁻², R³⁻¹⁻⁵ and R³⁻¹⁻⁴ are independently deuterium, cyano, halogen, hydroxyl, C₁₋₆ alkyl, C₂₋₆ alkenyl, C₁₋₆ alkyl-O—, C₁₋₆ alkyl-O—substituted with one or more halogen, C₁₋₆ alkyl substituted with one or more halogen, —C(═O)R^{e9}, —NR^{e10}R^{e11}, —C(═O)OR^{e12}, —C(═O)NR^{e13}R^{e14}, —OC(═O)NR^{e15}R^{e16}, “4- to 10-membered heterocycloalkyl containing 1-3 heteroatoms independently selected from O, S and N”, or, “4- to 10-membered heterocycloalkyl containing 1-3 heteroatoms independently selected from O, S and N” substituted with one or more R³⁻¹⁻¹⁻¹; or, two optional R³⁻¹⁻⁵ are connected, together with the atoms to which they are attached form “4- to 10-membered heterocycloalkyl containing 1-3 heteroatoms independently selected from O, S and N” substituted with one or more R³⁻¹⁻¹⁻²,
R³⁻¹⁻¹⁻¹ and R³⁻¹⁻¹⁻² are independently 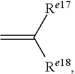
halogen, C₁₋₆ alkyl or C₆₋₂₀ aryl substituted with one or more R³⁻¹⁻¹⁻¹⁻¹; R³⁻¹⁻¹⁻¹⁻¹ is halogen or C₁₋₆ alkyl substituted with one or more halogen;
R^{e5}, R^{e6}, R^{e7}, R^{e8}, R^{e9}, R^{e1}, R^{e11}, R^{e12}, R^{e13}, R^{e14}, R^{e1}, R^{e16}, R^{e171}, R^{e172}, R^{e173} and R^{e174} are independently hydrogen or C₁₋₆ alkyl;
or, R¹² and R¹³, R¹⁵ and R¹⁶, R^{c3} and R^{c4}, R²² and R²³, R²⁵ and R²⁶, R^{4b1} and R^{4b2}, R^{4d1} and R^{4d2}, R^{4f1} and R^{4f2}, R⁴ⁱ and R^{4j}, R^{e1} and R^{e2}, R^{e3} and R^{e4}, R^{e5} and R^{e6}, R^{e7} and R^{e8}, R^{e10} and R^{e11}, R^{e13} and R^{e14}, R^{e15} and R^{e16}, R^{e173} and R^{e174} together with the atom N to which they are attached form “4- to 12-membered heterocycloalkyl containing 1-3 heteroatoms, of which one is N, the other independently selected from O, S and N”, or, “4- to 12-membered heterocycloalkyl containing 1-3 heteroatoms, of which one is N, the other independently selected from O, S and N” substituted with one or more R³⁻¹⁻³; R³⁻¹⁻³ is independently deuterium, cyano, halogen, hydroxyl, C₁₋₆ alkyl-O—, C₁₋₆ alkyl, C₁₋₆ alkyl substituted with one or more R³³, “4- to 7-membered heterocycloalkyl containing 1 or 2 heteroatoms independently selected from O, S and N” or —C(═O)OR³⁰.
R^{c5}l, R^{c52}, R¹¹, R²¹, R¹⁴, R¹⁵, R¹⁶, R²⁴, R²⁵, R²⁶, R²⁷, R²⁸ and R³⁰ are independently hydrogen or C₁₋₆ alkyl;
R²⁹ is hydrogen, C₁₋₆ alkyl or —NR^{m}Rⁿ;
R³¹ is hydrogen, C₁₋₆ alkyl, —OC₁₋₆ alkyl, —NR^{m}Rⁿ, —OC(═O)R³² or —C(═O)OC₁-₆ alkyl;
R³² and R³³ are independently —OC₁₋₆ alkyl or C₁₋₆ alkyl substituted with one or more —NR^{m}Rⁿ; R²², R²³, R^{m} and Rⁿ are independently hydrogen, C₁₋₆ alkyl or Boc.

2. The nitrogen-containing heterocyclic compound of formula I, the pharmaceutically acceptable salt thereof, a prodrug thereof, a stereoisomer thereof, a tautomer thereof or an isotopically labeled compound thereof according to claim 1, wherein, “ [symbol] ” represents a single or double bond; 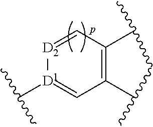
is phenyl, “5- to 7-membered heterocycloalkenyl containing 1-3 heteroatoms independently selected from O, S and N” or “5- to 7-membered heteroaryl containing 1-3 heteroatoms independently selected from O, S and N”; wherein D¹ is C, CH or N; D₂ is, 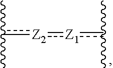
wherein Z₁ and Z₂ are independently a bond, CH, CH₂, O, S, N or NH;
r is 0, 1 or 2;
R⁵ is independently halogen, C₁₋₆ alkyl, C₁₋₆ alkyl substituted with one or more halogen, hydroxyl, C₁₋₆ alkyl-O—, C₂₋₆ alkenyl or nitro;
Y¹ is O, NH, CH₂ or 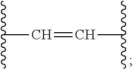 [SMILES: *C=C*]
Y² is NR² or O;
m1, m2 and n are independently 0, 1, 2, 3 or 4;
X¹ and X² are N;
R¹ is C₆₋₂₀ aryl, “5- to 12-membered heteroaryl containing 1-4 heteroatoms independently selected from O, S and N”, C₆₋₂₀ aryl substituted with one or more R¹⁻¹ or “5- to 12-membered heteroaryl containing 1-4 heteroatoms independently selected from O, S and N” substituted with one or more R¹⁻²; provided that when multiple substituents are present, the substituents are the same or different;
R¹⁻¹ and R¹⁻² are independently halogen, —OR^{c}, cyano, —NR¹²R¹³, C₁₋₆ alkyl, C₂₋₆ alkenyl, C₂₋₆ alkynyl, —B(OR^{x}) (OR^{y}) or C₁₋₆ alkyl substituted by one or more R¹⁻¹⁻¹; provided that when multiple substituents are present, the substituents are the same or different;
R², R^{c}, R¹² and R¹³ are independently hydrogen, C₁₋₆ alkyl, —C(═O)R^{c1}, —C(═O)OR^{c2}, —C(═O)NR^{c3}R^{c4}, —SO₂R^{c5}, 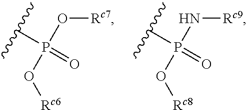
or C₁₋₆ alkyl substituted with one or more R^{c10};
R^{c1}R^{c2}, R^{c3}, R^{c4}, R^{c5}, R^{c6}, R^{c7}, R^{c8} and R^{c9} are independently hydrogen, C₁₋₂₀ alkyl, C₃₋₁₀ cycloalkyl, C₁₋₂₀ alkyl substituted with one or more R⁴⁻¹⁻¹, C₆₋₂₀ aryl substituted with one or more R⁴⁻¹⁻⁴, “5- to 7-membered heteroaryl containing 1 or 2 heteroatoms independently selected from O, S and N”, “5- to 7-membered heteroaryl containing 1 or 2 heteroatoms independently selected from O, S and N” substituted with one or more R⁴⁻¹⁻⁵, NR^{c51}R^{c52}, —(C₁-₆ alkylene-O)ₚ—C₁₋₆ alkyl, 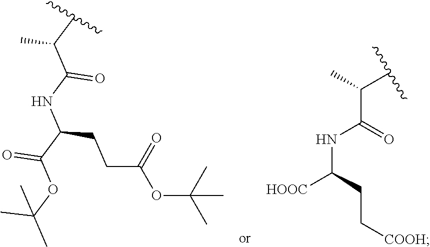 [SMILES: *[C@@H](C)C(=O)N[C@@H](CCC(=O)OC(C)(C)C)C(=O)OC(C)(C)C ; *[C@@H](C)C(=O)N[C@@H](CCC(=O)O)C(=O)O]
p is 1, 2, 3 or 4; provided that when multiple substituents are present, the substituents are the same or different;
R¹⁻¹⁻¹, R⁴⁻¹⁻¹R⁴⁻¹⁻⁴ and R⁴⁻¹⁻⁵ are independently halogen, hydroxyl, C₁₋₆ alkyl-O—, C₁₋₆ alkyl substituted with one or more R³¹, “4- to 7-membered heterocycloalkyl containing 1 or 2 heteroatoms independently selected from O, S, and N”, C₆₋₂₀ aryl, —C(═O)R²¹, —NR²²R²³, —C(═O)OR²⁴, —OC(═O)R³², —COOH, —C(═O)OR²⁷ or —OC(═O)OR²⁸;
R^{c10} is —OC₁₋₆ alkyl, hydroxyl, 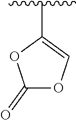 [SMILES: *c1coc(=O)o1]
substituted with C₁₋₆ alkyl, C₆₋₂₀ aryl substituted with one or more —B(OR^{x})(OR^{y}), “5- to 7-membered heteroaryl containing 1 or 2 heteroatoms independently selected from O, S, and
N” substituted with one or more R^{c10-1}, 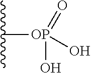
or —OC(═O)R²⁹; R^{c10-1} is independently C₁₋₆ alkyl or nitro;
or, optional R² and R⁵ together with the atoms to which they are attached, independently form “5- to 12-membered heterocyclic alkenyl containing 2 to 4 heteroatoms, two of which are N, and the other heteroatoms independently selected from O, S, and N”;
R^{x} and R^{y} are independently hydrogen or C₁₋₆ alkyl, or R^{x} and R^{y} each with the attached oxygen atom form five-membered ring substituted with one or more C₁₋₆ alkyl;
R⁴ is independently deuterium, halogen, hydroxyl, C₁-t alkyl, O═ or —C(═O)R^{4a}; or, when n is 2, 3 or 4, two optional R⁴ are connected, together with the atoms to which they are attached form “4- to 8-membered heterocyclic ring containing 1 to 3 heteroatoms independently selected from O, S, and N” substituted with one or more R⁴⁻⁵;
R⁴⁻⁵ is O═;
R^{4a} is C₁₋₆ alkyl or C₂₋₆ alkenyl;
L₁ is a bond or —O(R^{L-1})ₙ₁—; R^{L-1} is C₁₋₆ alkylene or C₁₋₆ alkylene substituted with one or more deuterium; n1 is 1;
R³ is C₃₋₁₂ cycloalkyl substituted with one or more R³⁻¹, “4- to 12-membered heterocycloalkyl containing 1-3 heteroatoms independently selected from O, S and N”, “4- to 12-membered heterocycloalkyl containing 1-3 heteroatoms independently selected from O, S and N” substituted with one or more R³⁻² or C₂₋₆ alkenyl substituted with one or more R³⁻⁴; provided that when multiple substituents are present, the substituents are the same or different;
R³⁻¹, R³⁻² and R³⁻⁴ are independently C₁₋₆ alkyl, C₁₋₆ alkyl substituted with one or more R³⁻¹⁻¹, C₆₋₂₀ aryl substituted
with one or more R³⁻¹⁻⁵, Ar, C₁₋₆ alkyl-O—, —CH₂—O—Ar, halogen or 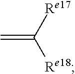
Ar is “5-12-membered heteroaryl containing 1-3 heteroatoms independently selected from O, N and S” substituted with one or more R³⁻¹⁻⁴;
R³⁻¹⁻¹, R³⁻¹⁻⁵ and R³⁻¹⁻⁴ are independently 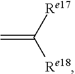
halogen, C₁₋₆ alkyl, C₂₋₆ alkenyl, C₁₋₆ alkyl-O—substituted with one or more halogen, C₁₋₆ alkyl substituted with one or more halogen, —C(═O)R^{e9}, —NR^{e10}R^{e11}, —OC(═O)NR^{e15}R^{e16}, “4- to 10-membered heterocycloalkyl containing 1-3 heteroatoms independently selected from O, S and N”, or, “4- to 10-membered heterocycloalkyl containing 1-3 heteroatoms independently selected from O, S and N” substituted with one or more R³⁻¹⁻¹⁻¹; or, two optional R³⁻¹⁻⁵ are connected, together with the atoms to which they are attached form “4- to 10-membered heterocycloalkyl containing 1-3 heteroatoms independently selected from O, S and N” substituted with one or more R³⁻¹⁻¹⁻².
R³⁻¹⁻¹⁻¹ and R³⁻¹⁻¹⁻² are independently 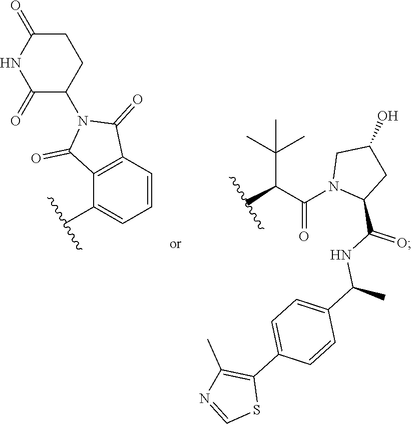 [SMILES: *c1cccc2c1C(=O)N(C1CCC(=O)NC1=O)C2=O ; *[C@H](C(=O)N1C[C@H](O)C[C@H]1C(=O)N[C@@H](C)c1ccc(-c2scnc2C)cc1)C(C)(C)C]
halogen, C₁₋₆ alkyl or C₆₋₂₀ aryl substituted with one or more R³⁻¹⁻¹⁻¹⁻¹; R³⁻¹⁻¹⁻¹⁻¹ is independently halogen or C₁₋₆ alkyl substituted with one or more halogen;
or, R³⁻¹ and R³⁻² are independently -L₂-M; L₂ is -L₂₁-L₂₂-L₂₃-L₂₄-L₂₅-L₂₆; L₂₁, L₂₃ and L₂₅ are independently a bond, O, S, NH, C(O)NH or C₁₋₆ alkylene; L₂₂ and L₂₄ are independently a bond, C₃₋₁₀ cycloalkyl, “4- to 7-membered heterocycloalkyl containing 1 or 2 heteroatoms independently selected from O, S and N”, C₆₋₂₀ aryl, or, “5- to 7-membered heteroaryl containing 1 or 2 heteroatoms independently selected from O, S, and N”; L₂₆ is a bond, O, S, NH or C(O)NH;
M is 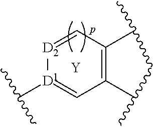
R^{e17} and R^{e18} are independently hydrogen or halogen;
R^{e9}, R^{e10}, R^{e11}, R^{e15} and R^{e16} are independently hydrogen or C₁₋₆ alkyl;
or, R^{c3} and R^{c4} together with the atom N to which they are attached form “4- to 12-membered heterocycloalkyl containing 1-3 heteroatoms, of which one is N, the other independently selected from O, S and N”, or, “4- to 10-membered heterocycloalkyl containing 1-3 heteroatoms, of which one is N, the other independently selected from O, S and N” substituted with one or more R³⁻¹⁻³; R³-¹-3 is independently C₁₋₆ alkyl substituted with one or more R^{33 “4}- to 7-membered heterocycloalkyl containing 1 or 2 heteroatoms independently selected from O, S and N” or —C(═O)OR³⁰;
R^{c51}, R^{c52}, R²⁴, R²⁷, R²⁸ and R³⁰ are independently hydrogen or C₁₋₆ alkyl;
R²⁹ is hydrogen, C₁₋₆ alkyl or —NR^{m}Rⁿ;
R³¹ is hydrogen, C₁₋₆ alkyl, —OC₁₋₆ alkyl, —NR^{m}Rⁿ, —OC(═O)R³² or —C(═O)OC₁₋₆ alkyl;
R³² and R³³ are independently-OC₁₋₆ alkyl or C₁₋₆ alkyl substituted with one or more —NR^{m}Rⁿ; R²¹, R²², R²³, R^{m} and
Rⁿ are independently hydrogen, C₁₋₆ alkyl or Boc.

3. The nitrogen-containing heterocyclic compound of formula I, the pharmaceutically acceptable salt thereof, a prodrug thereof, a stereoisomer thereof, a tautomer thereof or an isotopically labeled compound thereof according to claim 1, wherein, “ [symbol] ” represents a single or double bond;
is phenyl, “5- to 7-membered heterocycloalkenyl containing 1-3 heteroatoms independently selected from O, S and N” or “5- to 7-membered heteroaryl containing 1-3 heteroatoms independently selected from O, S and N”; wherein D¹ is C or N; D₂ is 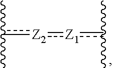
wherein Z₁ and Z₂ are independently a bond, CH, CH₂N or NH;
r is 0, 1 or 2;
R⁵ is independently halogen, C₁₋₆ alkyl, C₁₋₆ alkyl substituted with one or more halogen, hydroxyl or C₂₋₆ alkenyl;
Y¹ is O, NH, CH₂ or 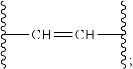 [SMILES: *C=C*]
Y² is NR² or O; R² is hydrogen, C₁₋₆ alkyl, —C(═O)R^{c1} or —C(═O)OR^{c2}; R^{c} is C₁₋₂₀ alkyl substituted with one or more R⁴⁻¹⁻¹; R^{c2} is C₁₋₆ alkyl; R⁴⁻¹⁻¹ is independently halogen;
m1 and m2 are independently 0 or 1;
n is 0, 1 or 2;
R⁴ is halogen, hydroxyl or C₁₋₆ alkyl;
X¹ and X² are N;
R¹ is C₆₋₂₀ aryl, “5- to 12-membered heteroaryl containing 1-4 heteroatoms independently selected from O, S and N”, C₆₋₂₀ aryl substituted with one or more R^{1′}—or “5- to 12-membered heteroaryl containing 1-4 heteroatoms independently selected from O, S and N” substituted with one or more R¹⁻²; provided that when multiple substituents are present, the substituents are the same or different;
R¹⁻¹ and R¹⁻² are independently halogen, —OR^{c}, cyano, —NR¹²R¹³, C₁₋₆ alkyl, C₂₋₆ alkenyl, C₂₋₆ alkynyl, —B(OR^{x}) (OR^{y}) or C₁₋₆ alkyl substituted by one or more R¹⁻¹⁻¹; provided that when multiple substituents are present, the substituents are the same or different;
R^{c} is hydrogen, —C(═O)R^{c1}, —C(═O)NR^{c3}R^{c4} or C₁₋₆ alkyl substituted with one or more R^{c10}; R^{c10} is independently —OC(═O)R²⁹; R²⁹ is —NR^{m}Rⁿ; R^{m} and Rⁿ are C₁₋₆ alkyl;
R^{c1}, R^{c3} and R^{c4} are independently hydrogen, C₁₋₂₀ alkyl, C₃₋₁₀ cycloalkyl, —(C₁₋₆ alkylene-O)ₚ—C₁₋₆ alkyl, C₁₋₂₀ alkyl substituted with one or more R⁴⁻¹⁻¹ or C₆₋₂₀ aryl substituted with one or more R⁴⁻¹⁻⁴; p is 1, 2 or 3;
R⁴⁻¹⁻¹ is independently C₁₋₆ alkyl-O—, —C(═O)R²¹, —NR²²R²³, —COOH or “4- to 7-membered heterocycloalkyl containing 1 or 2 heteroatoms independently selected from O, S, and N”; R²¹ is C₁₋₆ alkyl; R²² and R²³ are independently hydrogen, C₁₋₆ alkyl or Boc;
R⁴⁻¹⁻⁴ is halogen;
or, R^{c3} and R^{c4} together with the atom N to which they are attached form “4- to 12-membered heterocycloalkyl containing 1-3 heteroatoms, of which one is N, the other independently selected from O, S and N”, or, “4- to 12-membered heterocycloalkyl containing 1-3 heteroatoms, of which one is N, the other independently selected from O, S and N” substituted with one or more R³⁻¹⁻³; R³⁻¹⁻³ is independently C₁₋₆ alkyl substituted with one or more R³³ or “4- to 7-membered heterocycloalkyl containing 1 or 2 heteroatoms independently selected from O, S and N”;
R³³ is independently -OC₁-6 alkyl;
R¹² and R¹³ are independently hydrogen;
R^{x} and R^{y} are independently hydrogen;
R¹⁻¹⁻¹ is halogen;
L₁ is a bond or —O(R^{L-1})ₙ₁—; R^{L-1} is C₁₋₆ alkylene or C₁₋₆ alkylene substituted with one or more deuterium; n1 is 1;
R³ is C₃₋₁₂ cycloalkyl substituted with one or more R³⁻¹, “4- to 12-membered heterocycloalkyl containing 1-3 heteroatoms independently selected from O, S and N”, “4- to 12-membered heterocycloalkyl containing 1-3 heteroatoms independently selected from O, S and N” substituted with one or more R³⁻² or C₂₋₆ alkenyl substituted with one or more R³⁻⁴; provided that when multiple substituents are present, the substituents are the same or different;
R³⁻¹ is independently C₁₋₆ alkyl substituted with one or more R³⁻¹⁻¹; R³⁻¹⁻¹ is independently —NR^{e10}R^{e11}, “4- to 10-membered heterocycloalkyl containing 1-3 heteroatoms independently selected from O, S and N”, or, “4- to 10-membered heterocycloalkyl containing 1-3 heteroatoms independently selected from O, S and N” substituted with one or more R³⁻¹⁻¹⁻¹; R^{c10} and R^{c11} are independently C₁₋₆ alkyl; R³⁻¹⁻¹⁻¹ is independently 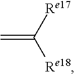
C₁₋₆ alkyl or C₆₋₂₀ aryl substituted with one or more R³⁻¹⁻¹⁻¹⁻¹; R³⁻¹⁻¹⁻¹⁻¹ is independently halogen;
R³⁻² is independently halogen, 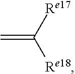
C₁₋₆ alkyl, C₁₋₆ alkyl substituted with one or more R³⁻¹⁻¹, Ar, —CH₂—O—Ar or C₆₋₂₀ aryl substituted with one or more R³⁻¹⁻⁵; Ar is “5-12-membered heteroaryl containing 1-3 heteroatoms independently selected from O, N and S” substituted with one or more R³⁻¹⁻⁴; R³⁻¹⁻¹ is halogen, C₂₋₆ alkenyl or —OC(═O)NR^{e15}R^{e16}; R^{e15} and R^{e16} are independently C₁₋₆ alkyl; R³⁻¹⁻⁵ and R³⁻¹⁻⁴ are independently C₁₋₆ alkyl, halogen, C₁₋₆ alkyl-O—substituted with one or more halogen or C₁₋₆ alkyl substituted with one or more halogen; or, two optional R³⁻¹⁻⁵ are connected, together with the atoms to which they are attached form “4- to 10-membered heterocycloalkyl containing 1-3 heteroatoms independently selected from O, S and N” substituted with one or more R³⁻¹⁻¹⁻²; R³⁻¹⁻¹⁻² is independently halogen;
or, R³⁻¹ is -L₂-M; L₂ is -L₂₁-L₂₂-L₂₃-L₂₄-L₂₅-L₂₆; L₂₁, L₂₃ and L₂₅ are independently a bond, O, S, NH, C(O)NH or C₁₋₆ alkylene; L₂₂ and L₂₄ are independently a bond, C₃₋₁₀ cycloalkyl, “4- to 7-membered heterocycloalkyl containing 1 or 2 heteroatoms independently selected from O, S and N”, C₆₋₂₀ aryl, or, “5- to 7-membered heteroaryl containing 1 or 2 heteroatoms independently selected from O, S, and N”; L₂₆ is a bond, O, S, NH or C(O)NH;
M is 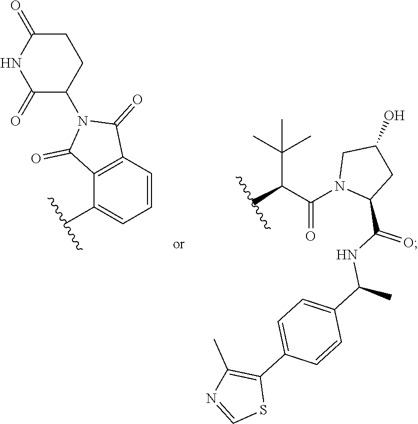 [SMILES: *c1cccc2c1C(=O)N(C1CCC(=O)NC1=O)C2=O ; *[C@H](C(=O)N1C[C@H](O)C[C@H]1C(=O)N[C@@H](C)c1ccc(-c2scnc2C)cc1)C(C)(C)C]
R³⁻⁴ is independently halogen or C₁₋₆ alkyl substituted with one or more R³⁻¹⁻¹; R³⁻¹⁻¹ is independently —NR^{e10}R^{e11} or “4- to 10-membered heterocycloalkyl containing 1-3 heteroatoms independently selected from O, S and N”;
R^{e10} and R^{e11} are independently C₁₋₆ alkyl;
R^{e17} and R^{e18} are independently hydrogen or halogen.

4. The nitrogen-containing heterocyclic compound of formula I, the pharmaceutically acceptable salt thereof, a prodrug thereof, a stereoisomer thereof, a tautomer thereof or an isotopically labeled compound thereof according to claim 1, wherein,
D¹ is C or N;
and/or, in D₂, one of Z₁ and Z₂ is CH, CH₂ or N; the other one is a bond;
and/or, r is 0, 1 or 2;
and/or, R⁵ is independently halogen, C₁₋₆ alkyl, C₁₋₆ alkyl substituted with one or more halogen, hydroxyl, C₁₋₆ alkyl-O—, C₂₋₆ alkenyl or nitro;
and/or, X¹ and X² are N;
and/or, R¹⁻¹ is independently halogen, —OR^{c}, C₁₋₆ alkyl, —NR¹²R¹³, C₂₋₆ alkynyl or C₁₋₆ alkyl substituted by one or more R¹⁻¹⁻¹; provided that when multiple substituents are present, the substituents are the same or different; R¹⁻¹⁻¹ is independently halogen;
and/or, R¹⁻² is independently halogen, cyano, C₂₋₆ alkenyl or —NR¹²R¹³;
and/or, R¹² and R¹³ are independently hydrogen, —SO₂R^{c5} or —C(═O)OR^{c2}; R^{c2} is C₁₋₆ alkyl;
and/or, R^{c} is hydrogen, —C(═O)R^{c1}, —C(═O)OR^{c2}, —C(═O)NR^{c3}R^{c4}, —SO₂R^{c5}, 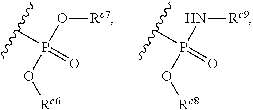
or C₁₋₆ alkyl substituted with one or more R^{c10}; R^{c10} is independently —OC₁₋₆ alkyl, 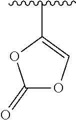 [SMILES: *c1coc(=O)o1]
substituted with C₁₋₆ alkyl, C₆₋₂₀ aryl substituted with one or more —B(OR^{x})(OR^{y}), “5- to 7-membered heteroaryl containing 1 or 2 heteroatoms independently selected from O, S, and N” substituted with one or more R¹⁰⁻¹¹, 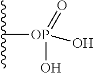
or —OC(═O)R²⁹; R^{c10-1} is independently C₁₋₆ alkyl or nitro; R^{c}, R^{c2}, R^{c3}, R^{c4}, R^{c5}, R^{c6}, R^{c7}, R^{c8} and R^{c9} are independently hydrogen, C₁₋₂₀ alkyl, C₃₋₁₀ cycloalkyl, C₁₋₂₀ alkyl substituted with one or more R⁴⁻¹⁻¹, C₆₋₂₀ aryl substituted with one or more R⁴⁻¹⁻⁴, “5- to 7-membered heteroaryl containing 1 or 2 heteroatoms independently selected from O, S and N”, “5- to 7-membered heteroaryl containing 1 or 2 heteroatoms independently selected from O, S and N” substituted with one or more R⁴⁻¹⁻⁵, NR^{c51}R^{c52}, —(C₁₋₆ alkylene-O)ₚ—C₁₋₆ alkyl, 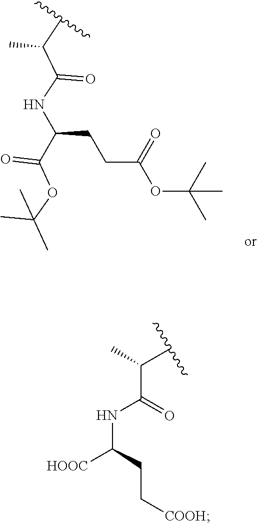 [SMILES: *[C@@H](C)C(=O)N[C@@H](CCC(=O)OC(C)(C)C)C(=O)OC(C)(C)C ; *[C@@H](C)C(=O)N[C@@H](CCC(=O)O)C(=O)O]
p is 1, 2, 3 or 4; provided that when multiple substituents are present, the substituents are the same or different; R¹⁻¹, R⁴⁻¹⁻¹, R⁴-¹-4 and R⁴⁻¹⁻⁵ are independently halogen, hydroxyl, C₁₋₆ alkyl-O—, C₁₋₆ alkyl, C₁₋₆ alkyl substituted with one or more R³¹, “4- to 7-membered heterocycloalkyl containing 1 or 2 heteroatoms independently selected from O, S, and N”, C₆₋₂₀ aryl, “5- to 7-membered heteroaryl containing 1 or 2 heteroatoms independently selected from O, S and N”, —NR²²R²³, —C(═O)OR²⁴, —OC(═O)R³², —COOH, —C(═O)OR²⁷ or —OC(═O)OR²⁸; R^{x} and R^{y} are independently hydrogen, or R^{x} and R^{y} each with the attached oxygen atom form five-membered ring substituted with one or more C₁₋₆ alkyl;
or, R^{c3} and R^{c4} together with the atom N to which they are attached form “4- to 12-membered heterocycloalkyl containing 1-3 heteroatoms, of which one is N, the other independently selected from O, S and N”, or, “4- to 12-membered heterocycloalkyl containing 1-3 heteroatoms, of which one is N, the other independently selected from O, S and N” substituted with one or more R³⁻¹⁻³; R³-¹⁻³ is independently C₁₋₆ alkyl substituted with one or more R^{33 “4}- to 7-membered heterocycloalkyl containing 1 or 2 heteroatoms independently selected from O, S and N” or —C(═O)OR³⁰; R³⁰ is hydrogen or C₁₋₆ alkyl; R³³ is independently-OC₁₋₆ alkyl or C₁₋₆ alkyl substituted with one or more —NR^{m}Rⁿ; R^{m} and Rⁿ are independently hydrogen, C₁₋₆ alkyl or Boc;
and/or, when R¹ is C₆₋₂₀ aryl substituted with one or more R¹⁻¹, the more is two or three;
and/or, Y¹ is O, NH, CH₂ or 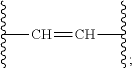 [SMILES: *C=C*]
and/or, m1, m2 and n are independently 0, 1 or 2;
and/or, Y² is O or NR²;
and/or, R² is hydrogen, C₁₋₆ alkyl, —C(═O)R^{c1} or —C(═O)OR^{c2}; R^{c1} is C₁₋₂₀ alkyl or C₁₋₂₀ alkyl substituted with one or more R⁴⁻¹⁻¹; R^{c2} is C₁₋₆ alkyl or C₁₋₆ alkyl substituted with one or more R⁴⁻¹⁻¹; R⁴⁻¹⁻¹ is independently halogen, C₆₋₂₀ aryl or —OC(═O)R²⁴; or, optional R² and R⁵ together with the atoms to which they are attached, independently form “5- to 12-membered heterocyclic alkenyl containing 2 to 4 heteroatoms, two of which are N, and the other heteroatoms independently selected from O, S, and N”;
and/or, R²⁴ is C₁₋₆ alkyl;
and/or, R⁴ is independently halogen, hydroxyl, deuterium, —C(═O)R^{4a}, O═ or C₁₋₆ alkyl; R^{4a} is C₁₋₆ alkyl or C₂₋₆ alkenyl;
and/or, L₁ is a bond or —O(R^{L-1})ₙ₁—; R^{L-1} is C₁₋₆ alkylene or C₁₋₆ alkylene substituted with one or more deuterium;
n1 is 1;
and/or, R³ is C₃₋₁₂ cycloalkyl substituted with one or more R³⁻¹, “4- to 12-membered heterocycloalkyl containing 1-3 heteroatoms independently selected from O, S and N”, “4- to 12-membered heterocycloalkyl containing 1-3 heteroatoms independently selected from O, S and N” substituted with one or more R³⁻² or C₂₋₆ alkenyl substituted with one or more R³⁻⁴; provided that when multiple substituents are present, the substituents are the same or different;
and/or, R³⁻¹ is independently C₁₋₆ alkyl substituted with one or more R³⁻¹⁻¹; R³⁻¹⁻¹ is independently —NR^{e10}R^{e11}, “4- to 10-membered heterocycloalkyl containing 1-3 heteroatoms independently selected from O, S and N”, or, “4- to 10-membered heterocycloalkyl containing 1-3 heteroatoms independently selected from O, S and N” substituted with one or more R³⁻¹⁻¹⁻¹; R^{c10} and R^{c11} are independently 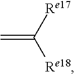
C₁₋₆ alkyl; R³⁻¹⁻¹ is independently R^{e18}, C₁₋₆ alkyl or C₆₋₂₀ aryl substituted with one or more R³⁻¹⁻¹⁻¹⁻¹; R³-¹⁻¹⁻¹⁻¹ is independently halogen or C₁₋₆ alkyl substituted with one or more halogen; R^{e17} and R^{e18} are independently hydrogen or halogen; or, R³⁻¹ is -L₂-M; L₂ is -L₂₁-L₂₂-L₂₃-L₂₄-L₂₅-L₂₆; L₂₁, L₂₃ and L₂₅ are independently a bond, O, S, NH, C(O)NH or C₁₋₆ alkylene; L₂₂ and L₂₄ are independently a bond, C₃₋₁₀ cycloalkyl, “4- to 7-membered heterocycloalkyl containing 1 or 2 heteroatoms independently selected from O, S and N”, C₆₋₂₀ aryl, or, “5- to 7-membered heteroaryl containing 1 or 2 heteroatoms independently selected from O, S, and N”; L₂₆ is a bond, O, S, NH or C(O)NH; M is 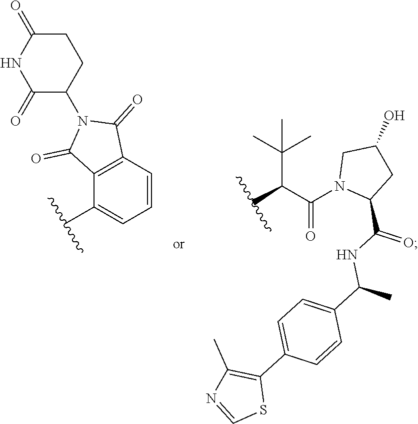 [SMILES: *c1cccc2c1C(=O)N(C1CCC(=O)NC1=O)C2=O ; *[C@H](C(=O)N1C[C@H](O)C[C@H]1C(=O)N[C@@H](C)c1ccc(-c2scnc2C)cc1)C(C)(C)C]
and/or, R³⁻² is independently halogen, C₁₋₆ alkyl, 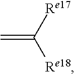
C₁₋₆ alkyl substituted with one or more R³⁻¹⁻¹ Ar, —CH₂—O—Ar, or C₆₋₂₀ aryl substituted with one or more R³⁻¹⁻⁵; Ar is “5-12-membered heteroaryl containing 1-3 heteroatoms independently selected from O, N and S” substituted with one or more R³⁻¹⁻⁴; R³⁻¹⁻¹ is halogen, C₂₋₆ alkenyl or —OC(═O)NR^{e15}R^{e16}; R^{c15} and R^{e16} are independently C₁₋₆ alkyl; R^{e17} and R^{c18} are independently hydrogen or halogen; R³⁻¹⁻⁵ and R³⁻¹⁻⁴ are independently C₁₋₆ alkyl, halogen, C₁₋₆ alkyl-O—substituted with one or more halogen or C₁₋₆ alkyl substituted with one or more halogen; or, two optional R³⁻¹⁻⁵ are connected, together with the atoms to which they are attached form “4- to 10-membered heterocycloalkyl containing 1-3 heteroatoms independently selected from O, S and N” substituted with one or more R³⁻¹⁻¹⁻²; R³⁻¹⁻¹⁻² is independently halogen;
and/or, R³⁻⁴ is independently halogen or C₁₋₆ alkyl substituted with one or more R³⁻¹⁻¹; R³⁻¹⁻¹ is independently —NR^{e10}R^{e11} or “4- to 10-membered heterocycloalkyl containing 1-3 heteroatoms independently selected from O, S and N”; R^{e10} and R^{e11} are independently C₁₋₆ alkyl.

5. The nitrogen-containing heterocyclic compound of formula I, the pharmaceutically acceptable salt thereof, a prodrug thereof, a stereoisomer thereof, a tautomer thereof or an isotopically labeled compound thereof
according to claim 1, wherein, when r is 1, 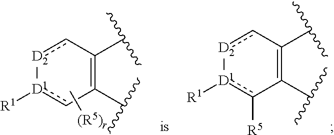
and/or, inland m2 are independently is 0 or 1;
and/or, R^{e17} and R^{e18} are independently is hydrogen or halogen;
and/or, when the definition of R⁵, R^{b}, R¹⁻¹, R¹⁻², R¹⁻¹⁻¹, R¹⁻¹⁻², R¹⁻¹⁻³, R¹⁻¹⁻⁴R¹⁻¹⁻⁵R¹⁻¹⁻⁶R⁴⁻¹⁻¹R⁴⁻¹⁻²R⁴⁻¹⁻³, R⁴⁻¹⁻⁴, R⁴⁻¹⁻⁵, R⁴, R⁴⁻¹, R⁴⁻², R⁴⁻³, R⁴⁻⁴, R⁴⁻⁵, R^{a}, R³⁻¹, R³⁻², R³⁻³, R³⁻⁴, R³⁻¹⁻¹, R³⁻¹⁻², R³⁻¹⁻³, R³⁻¹⁻⁵R³⁻¹⁻⁴, R^{4g}, R^{4h}, R^{e17}, R^{e18}, R³⁻¹⁻¹⁻¹ and R³⁻¹⁻¹⁻¹⁻¹ mentions halogen, the halogen is fluorine, chlorine, bromine or iodine;
and/or, when the definition of R¹, R¹⁻¹, R¹⁻², R, R^{c2}, R^{c3}, R^{c4}, R^{c5}, R^{c6}, R^{c7}, R^{c8}, R^{c9}, R¹⁻¹⁻¹, R¹⁻¹⁻², R¹⁻¹⁻³, R¹⁻¹⁻⁴, R¹⁻¹⁻⁵,R¹⁻¹⁻⁶, R⁴⁻¹⁻¹, R⁴⁻¹⁻², R⁴⁻¹⁻³, R⁴⁻¹⁻⁴, R⁴⁻¹⁻⁵, R^{c10}, R³⁻¹, R³⁻², R³⁻³, R³⁻⁴ and R³⁻¹⁻¹⁻¹ mentions C₆₋₂₀ aryl, the C_{6- 20} aryl is phenyl or naphthyl;
and/or, when the definition of R⁵, R¹⁻¹, R¹⁻², R², R^{c}R¹², R¹³, R^{c10}, R^{c10-1}, R¹⁻¹⁻¹, R¹⁻¹⁻²R¹⁻¹⁻³, R¹⁻¹⁻⁴, R¹⁻¹⁻⁵, R¹⁻¹⁻⁶, R⁴⁻¹⁻¹R⁴⁻¹⁻²R⁴⁻¹⁻³, R⁴⁻¹⁻⁴, R⁴⁻¹⁻⁵, R^{c51}, R^{c52}, R¹¹, R²¹, R²², R²³, R¹⁴, R²⁴, R¹⁵, R²⁵, R¹⁶, R²⁶, R²⁷, R²⁸, R²⁹, R³⁰, R³¹, R³², R³³, R^{x}, R_{y}, R⁴, R⁴⁻¹, R⁴⁻², R⁴⁻³, R⁴⁻⁴, R⁴⁻⁵, R^{a}, R^{4a}, R^{4b1}, R^{4b2}, R^{4c}, R^{4d1}, R^{4d2}, R^{4c}, R⁴¹, R^{4f2}, R⁴ⁱ, R^{4j}, R^{L-3}, R³, R³⁻¹, R³⁻², R³⁻³, R³⁻⁴, R^{4g}, R^{4h}, R^{e17}, R^{e18}, R^{d}, R^{d1}, R^{e1}, R^{e2}, R^{e3}, R^{e4}, R³⁻¹⁻¹, R³⁻¹⁻², R³⁻¹⁻⁵, R^{3-1- 4}, R³⁻¹⁻¹⁻¹, R³⁻¹⁻¹⁻¹⁻¹, R^{e5}, R^{e6}, R^{e7}, R^{e8}, R^{e9}, R^{e10}, R^{e11}, R^{e12}, R^{e13}, R^{e14}, R^{e15}, R^{e16}, R^{e171}, R^{e172}, R^{e173}, R^{e174} and R³⁻¹⁻³ mentions C₁₋₆ alkyl, the C₁₋₆ alkyl is C₁₋₄ alkyl, for example, methyl, ethyl, n-propyl, isopropyl, n-butyl, sec-butyl, isobutyl or tert-butyl;
and/or, when the definition of R^{c1}R^{c2}, R^{c3}, R^{d4}, R^{c5}, R^{c6}, R^{c7}, R^{c8} and R^{c9} independently is C₁₋₂₀ alkyl, the C₁₋₂₀ alkyl is C₁₋₁₂ alkyl, for example, methyl, ethyl, n-propyl, isopropyl, n-butyl, sec-butyl, isobutyl or tert-butyl;
and/or, when the definition of R¹⁻¹, R¹⁻² and R⁴ mentions C₂₋₆ alkynyl, the C₂₋₆ alkynyl is ethynyl, propynyl or propargyl;
and/or, when the definition of R⁵, R¹⁻¹, R¹⁻², R⁴, R⁴⁻¹, R⁴⁻², R⁴⁻³, R⁴⁻⁴, R⁴⁻⁵, R^{a}, R^{4a}, R^{4b1}, R^{4b2}, R^{4c}, R^{4d1}, R^{4d2}, R^{4e}, R^{4f1}, R^{4f2}, R⁴ⁱ, R^{4j}, R³, R³⁻¹⁻¹, R³⁻¹⁻², R³⁻¹⁻⁵ and R³⁻¹⁻⁴ mentions C₂₋₆ alkenyl, the C₂₋₆ alkenyl is vinyl, propenyl or allyl.

6. The nitrogen-containing heterocyclic compound of formula I, the pharmaceutically acceptable salt thereof, a prodrug thereof, a stereoisomer thereof, a tautomer thereof or an isotopically labeled compound thereof according to claim 1, wherein,
R⁴ is independently methyl, F, D, 0xo, 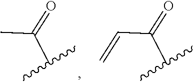
or hydroxyl;
and/or 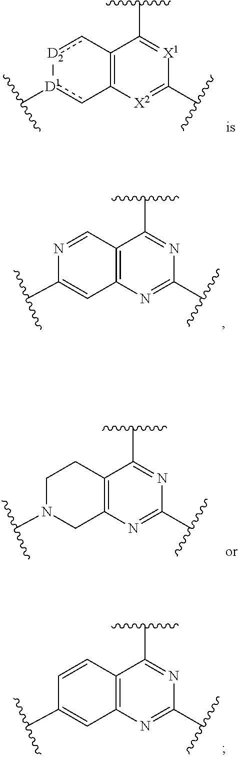
and/or, R⁵ is independently —F, —OH, —Cl, —CF₃, 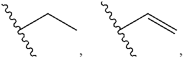
—NO₂, -OEt or —OMe, or, R² and R⁵ form 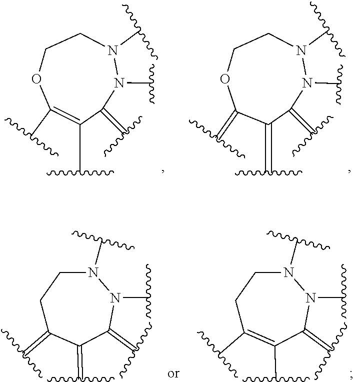 [SMILES: */C1=C(\*)C(=*)N(*)N(*)CCO1 ; *N1CCOC(=*)C(=*)C(=*)N1* ; *N1CCC(=*)C(=*)C(=*)N1* ; *C1=C(*)C(=*)N(*)N(*)CC1]
and/or R¹ is 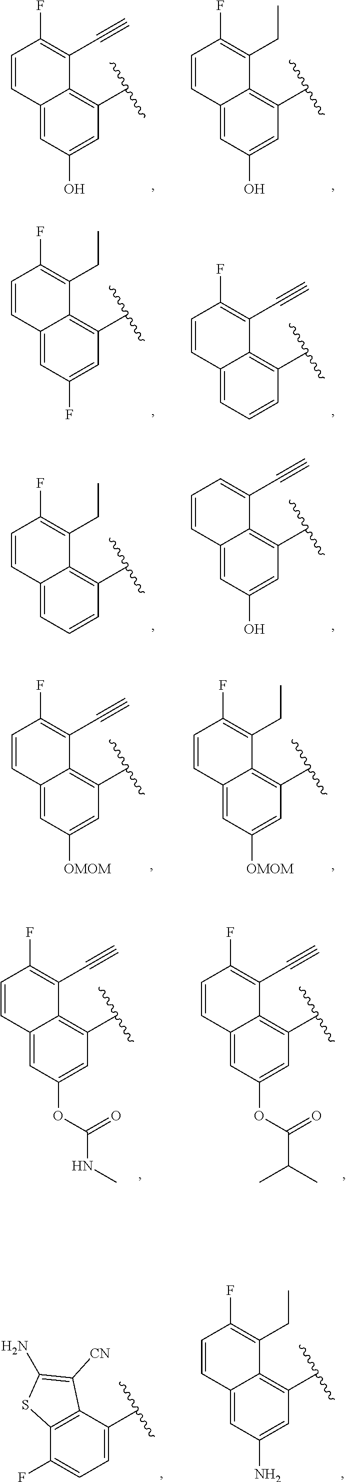 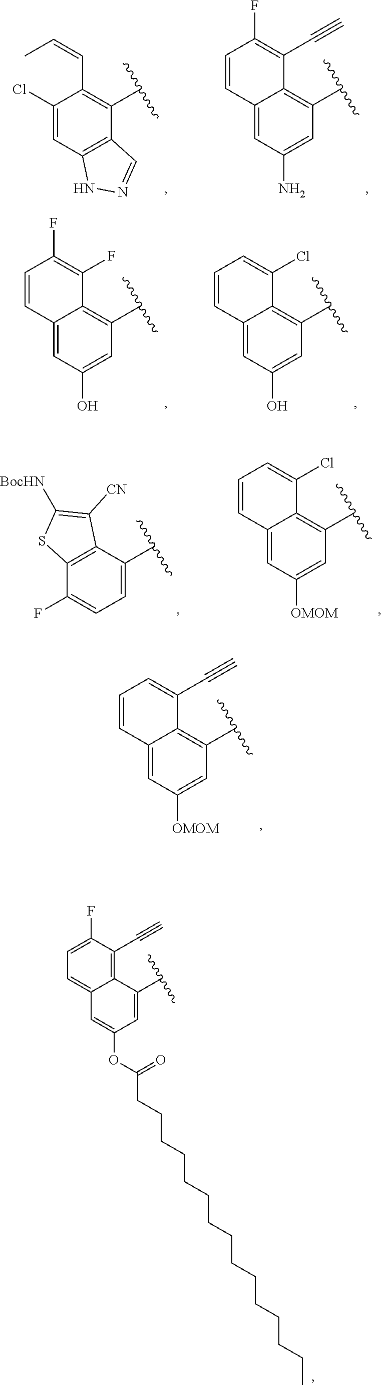 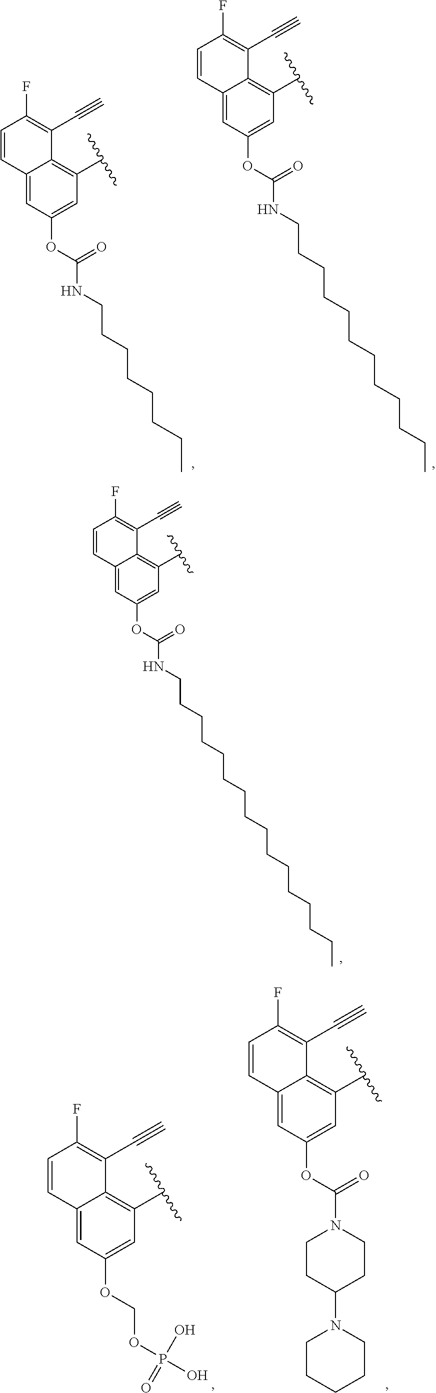 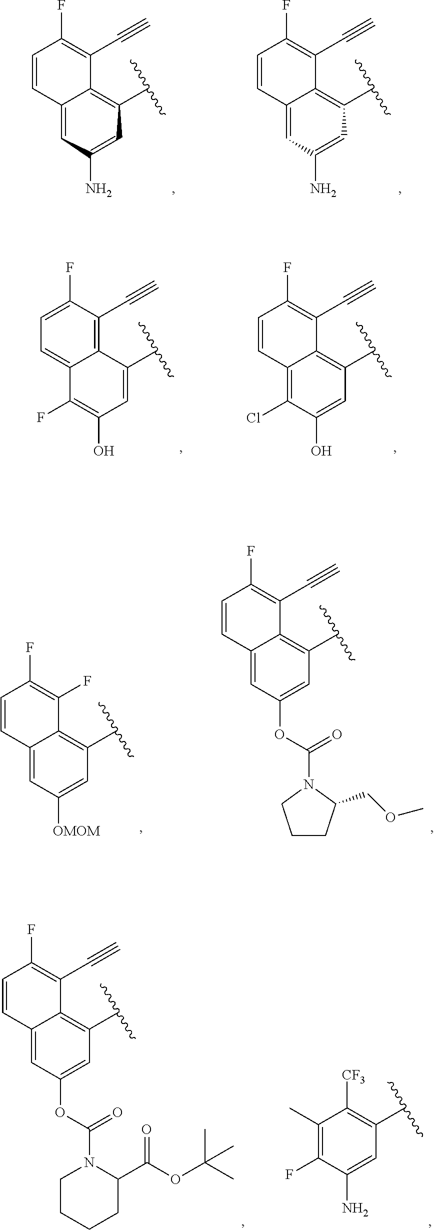 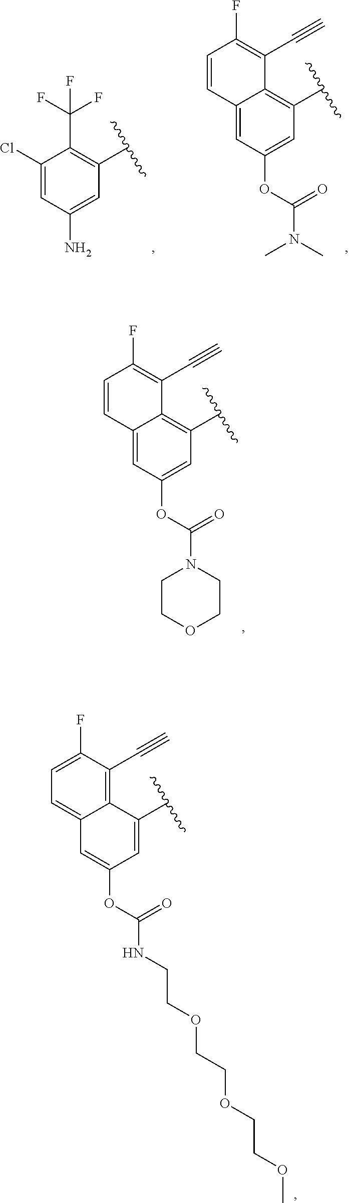 [SMILES: *c1cc(N)cc(Cl)c1C(F)(F)F ; *c1cc(OC(=O)N(C)C)cc2ccc(F)c(C#C)c12 ; *c1cc(OC(=O)N2CCOCC2)cc2ccc(F)c(C#C)c12 ; *c1cc(OC(=O)NCCOCCOCCOC)cc2ccc(F)c(C#C)c12] 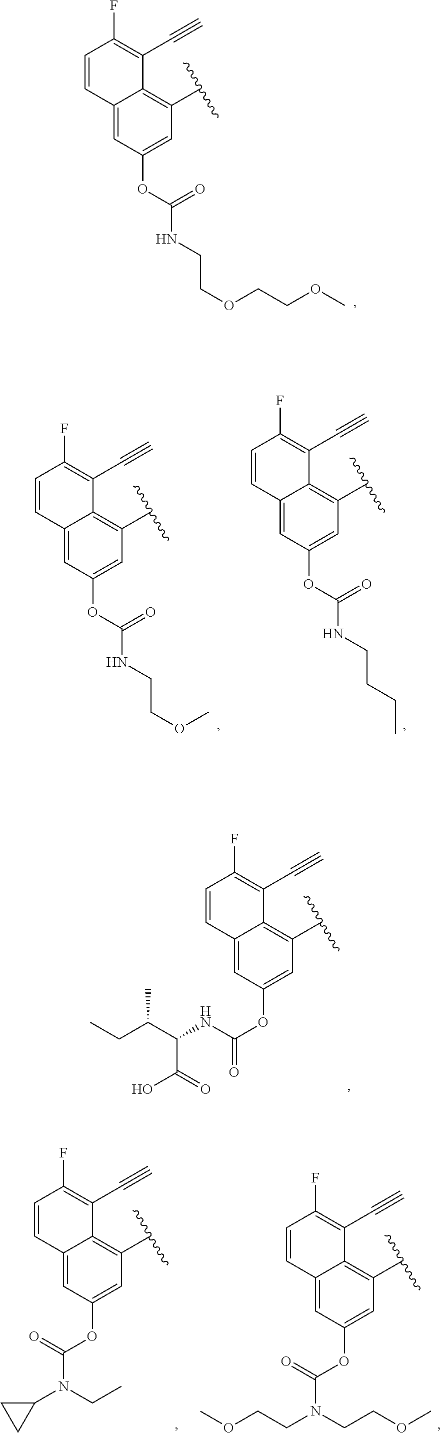 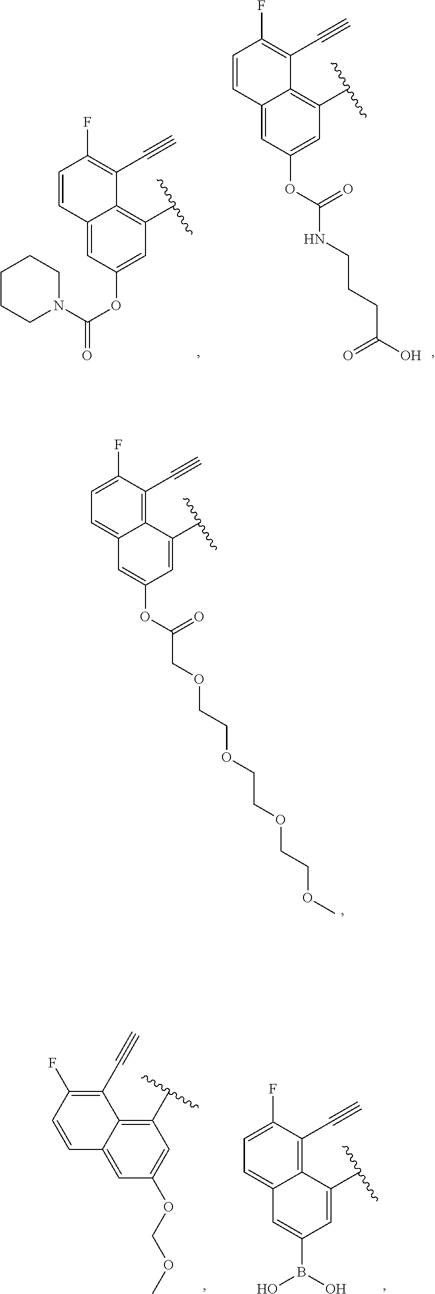 [SMILES: *c1cc(OC(=O)N2CCCCC2)cc2ccc(F)c(C#C)c12 ; *c1cc(OC(=O)NCCCC(=O)O)cc2ccc(F)c(C#C)c12 ; *c1cc(OC(=O)COCCOCCOCCOC)cc2ccc(F)c(C#C)c12 ; *c1cc(OCOC)cc2ccc(F)c(C#C)c12 ; *c1cc(B(O)O)cc2ccc(F)c(C#C)c12] 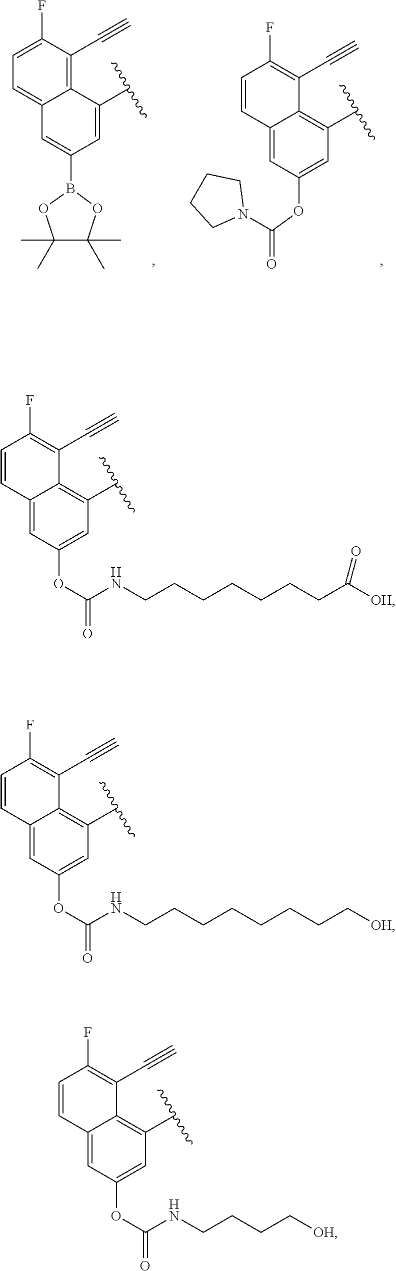 [SMILES: *c1cc(B2OC(C)(C)C(C)(C)O2)cc2ccc(F)c(C#C)c12 ; *c1cc(OC(=O)N2CCCC2)cc2ccc(F)c(C#C)c12 ; *c1cc(OC(=O)NCCCCCCCC(=O)O)cc2ccc(F)c(C#C)c12 ; *c1cc(OC(=O)NCCCCCCCCO)cc2ccc(F)c(C#C)c12 ; *c1cc(OC(=O)NCCCCO)cc2ccc(F)c(C#C)c12] 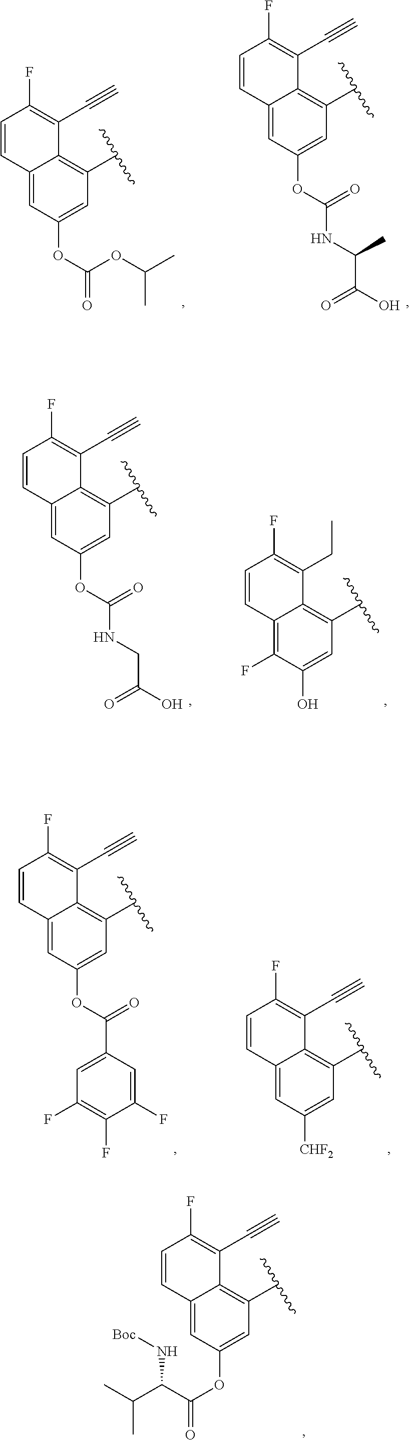 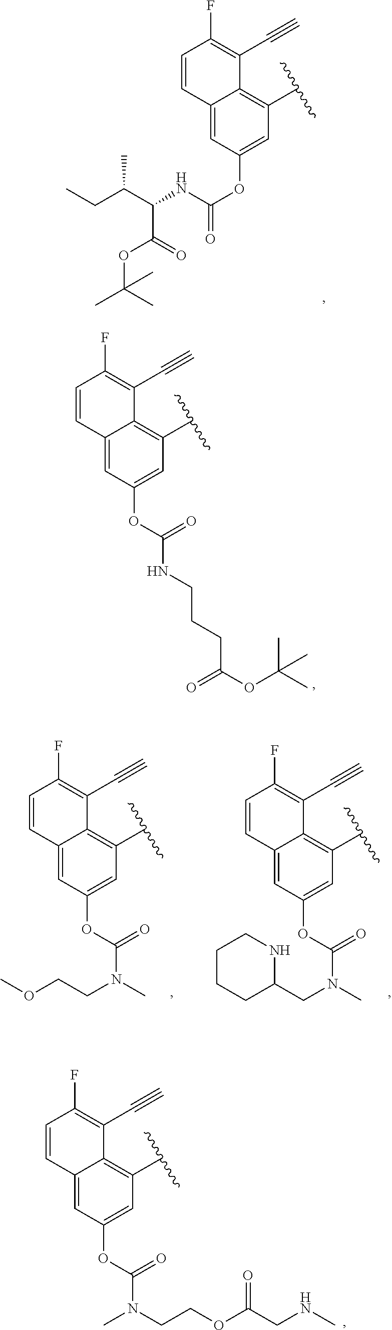 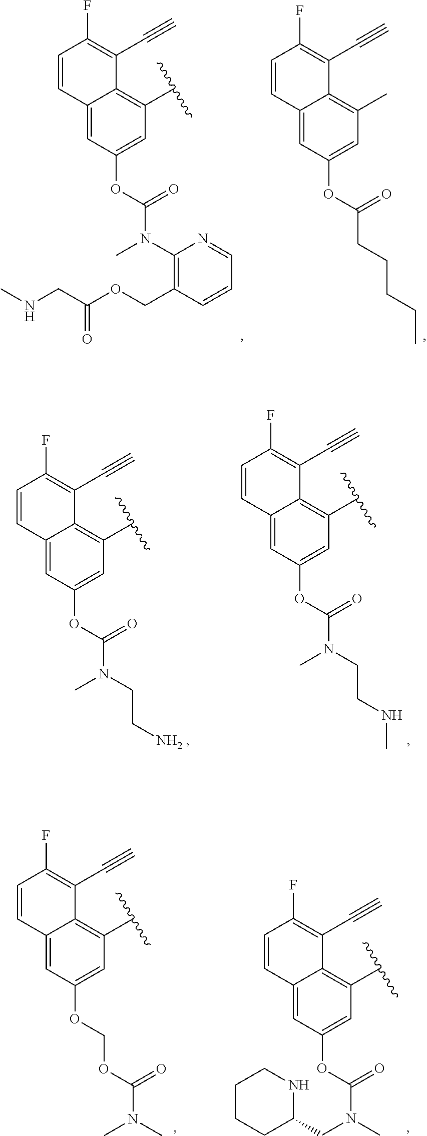 [SMILES: *c1cc(OC(=O)N(C)c2ncccc2COC(=O)CNC)cc2ccc(F)c(C#C)c12 ; C#Cc1c(F)ccc2cc(OC(=O)CCCCC)cc(C)c12 ; *c1cc(OC(=O)N(C)CCN)cc2ccc(F)c(C#C)c12 ; *c1cc(OC(=O)N(C)CCNC)cc2ccc(F)c(C#C)c12 ; *c1cc(OCOC(=O)N(C)C)cc2ccc(F)c(C#C)c12 ; *c1cc(OC(=O)N(C)C[C@@H]2CCCCN2)cc2ccc(F)c(C#C)c12] 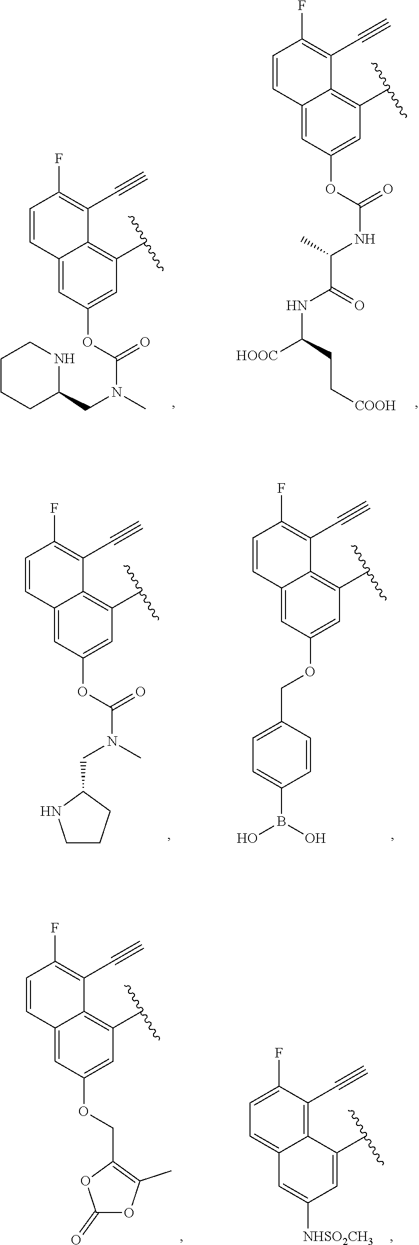 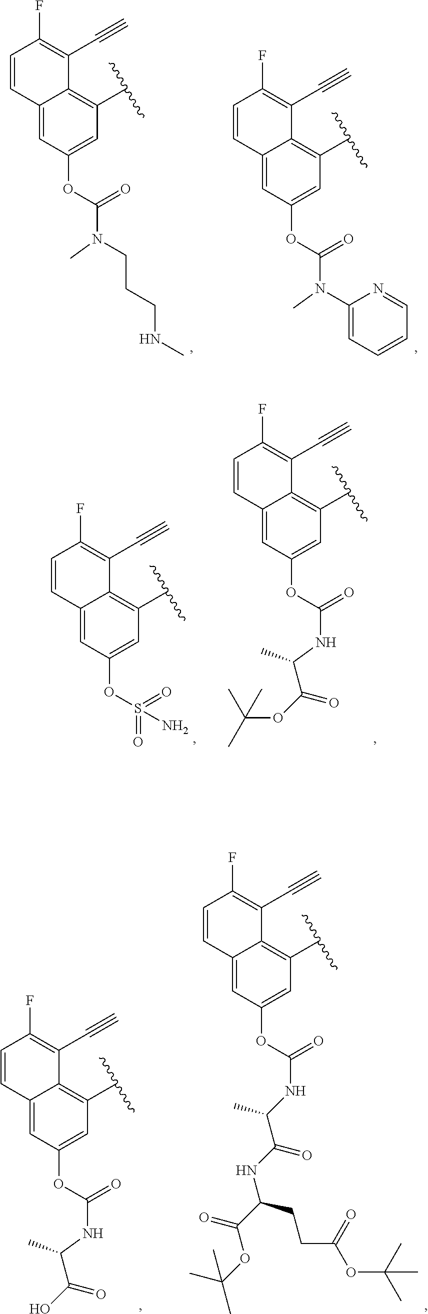 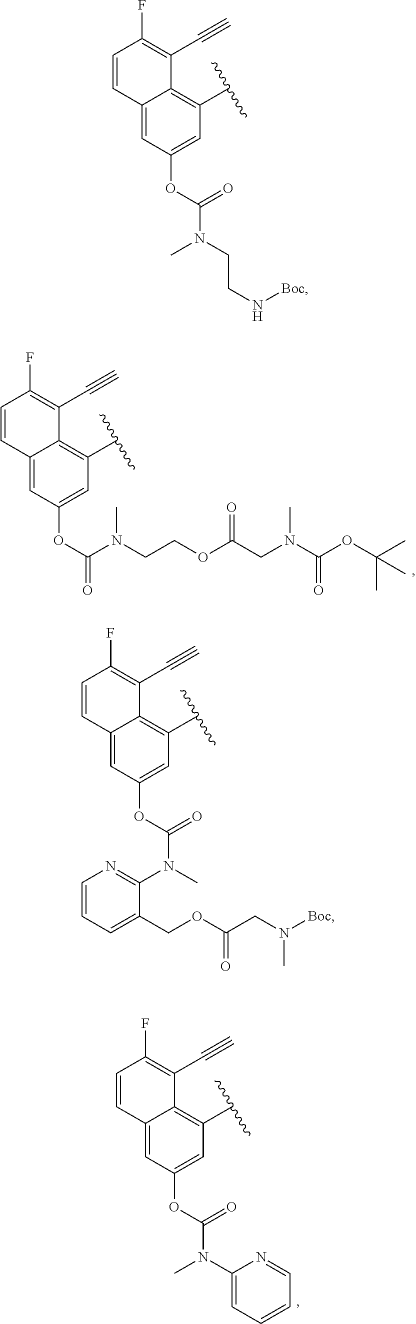 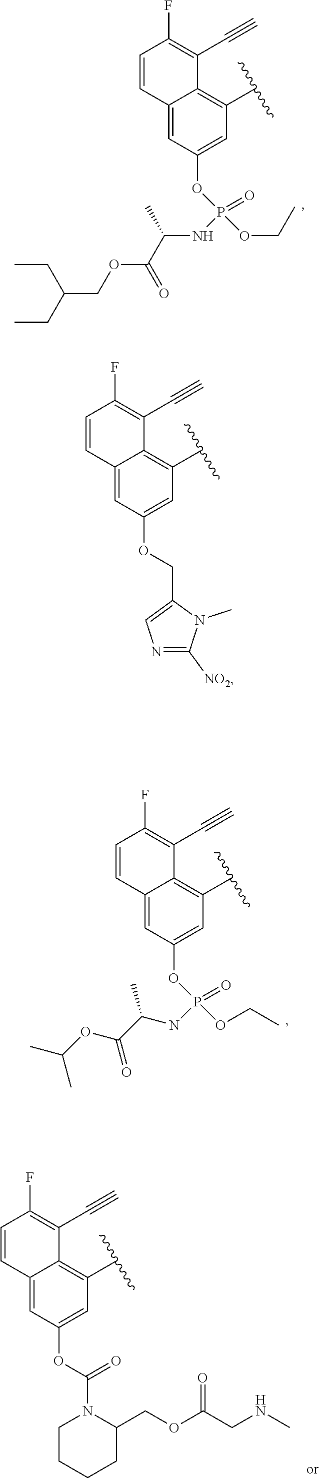 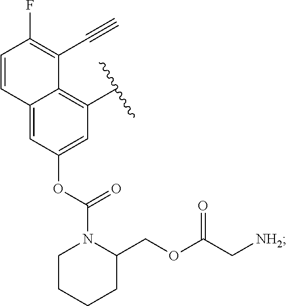
and/or, L₁ is a bond, —OCH₂—or -OCD₂-;
and/or, R³ is

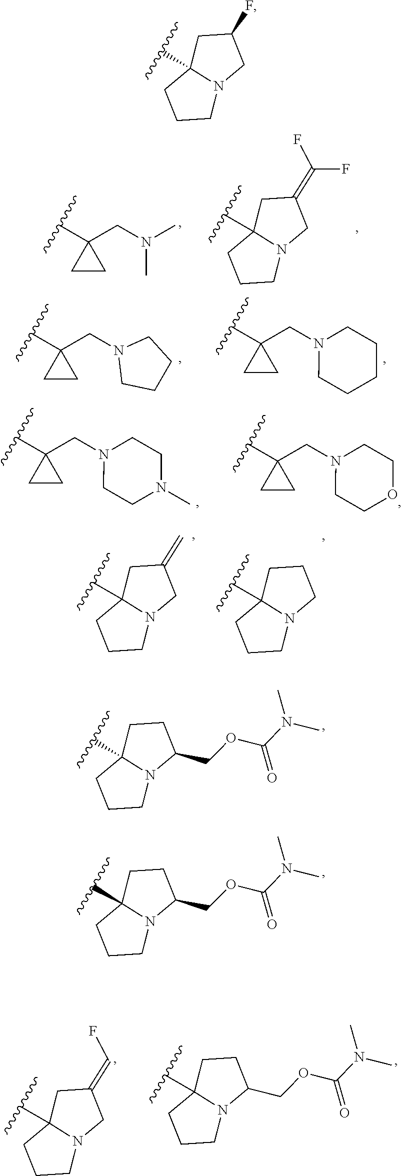

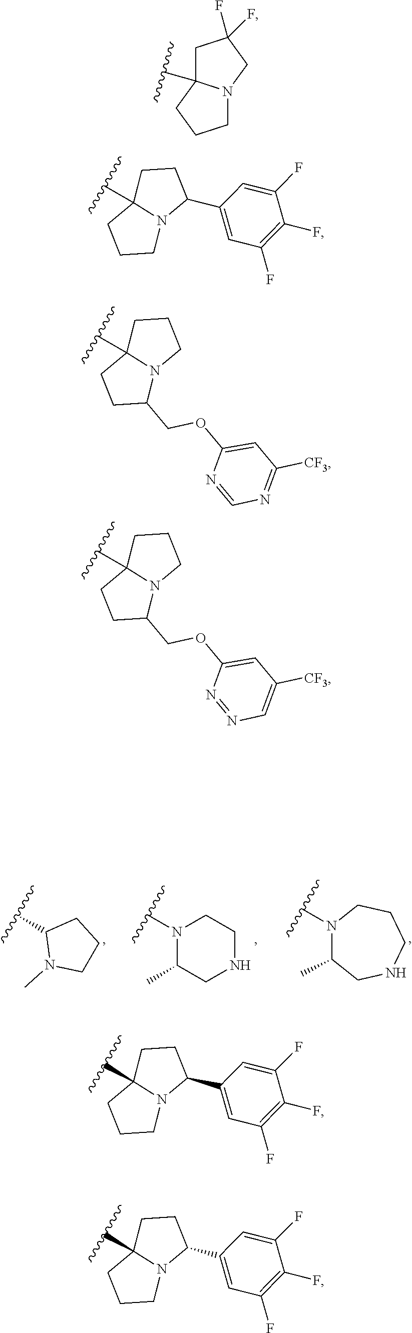

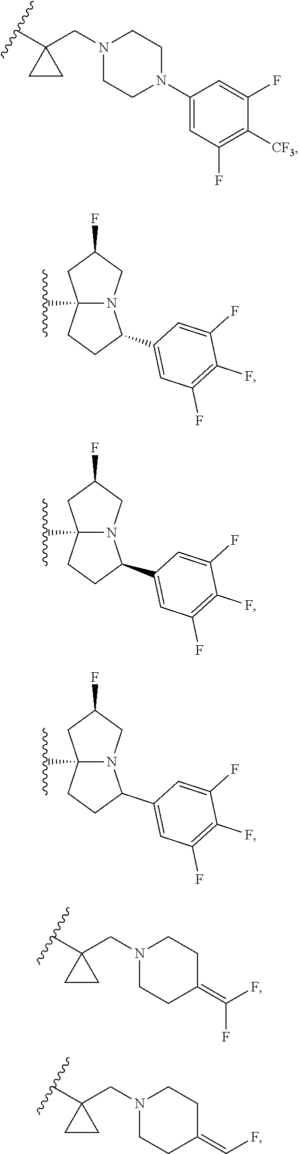 [SMILES: *C1(CN2CCN(c3cc(F)c(C(F)(F)F)c(F)c3)CC2)CC1 ; *[C@@]12CC[C@@H](c3cc(F)c(F)c(F)c3)N1C[C@H](F)C2 ; *[C@@]12CC[C@H](c3cc(F)c(F)c(F)c3)N1C[C@H](F)C2 ; *[C@@]12CCC(c3cc(F)c(F)c(F)c3)N1C[C@H](F)C2 ; *C1(CN2CCC(=C(F)F)CC2)CC1 ; *C1(CN2CCC(=CF)CC2)CC1]

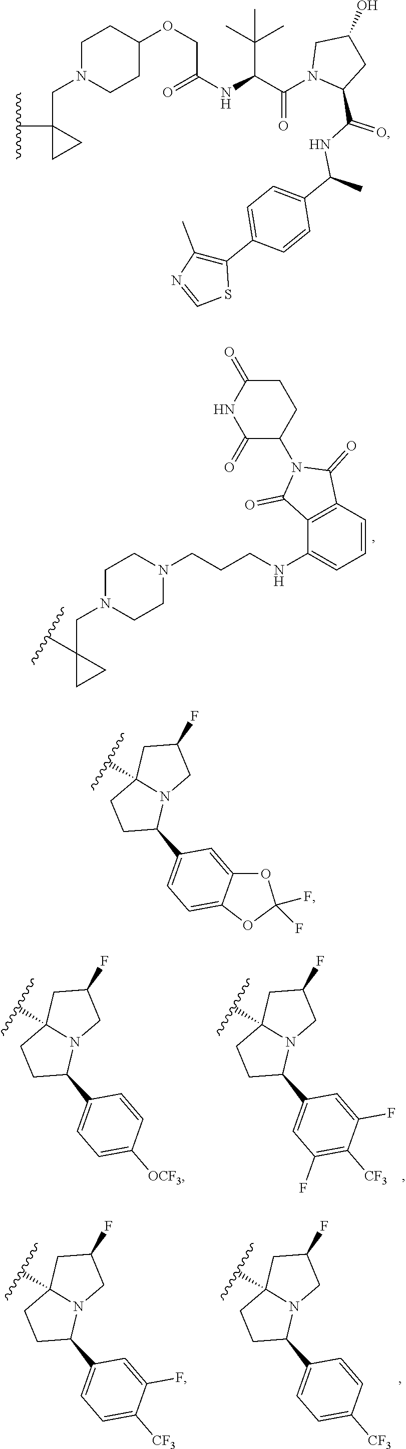

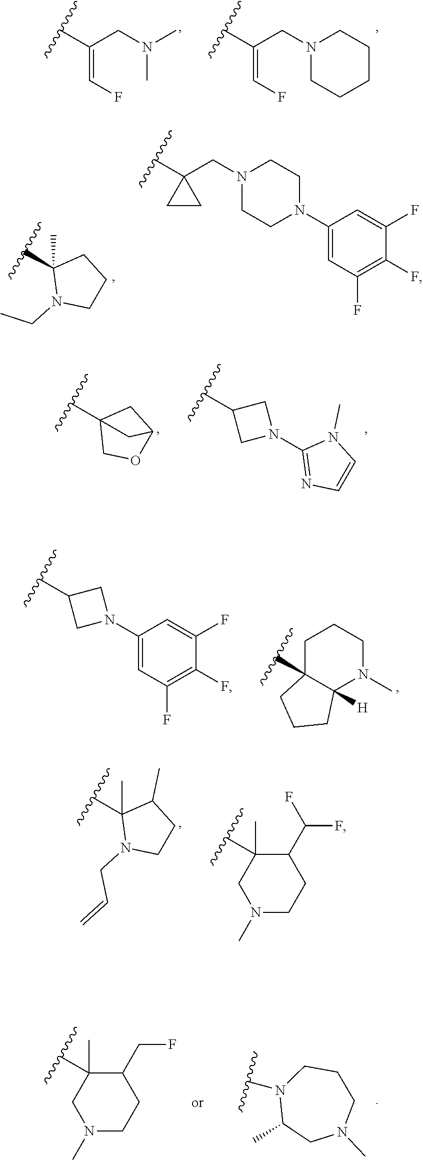

7. The nitrogen-containing heterocyclic compound of formula I, the pharmaceutically acceptable salt thereof, a prodrug thereof, a stereoisomer thereof, a tautomer thereof or an isotopically labeled compound thereof according to claim 1, wherein,

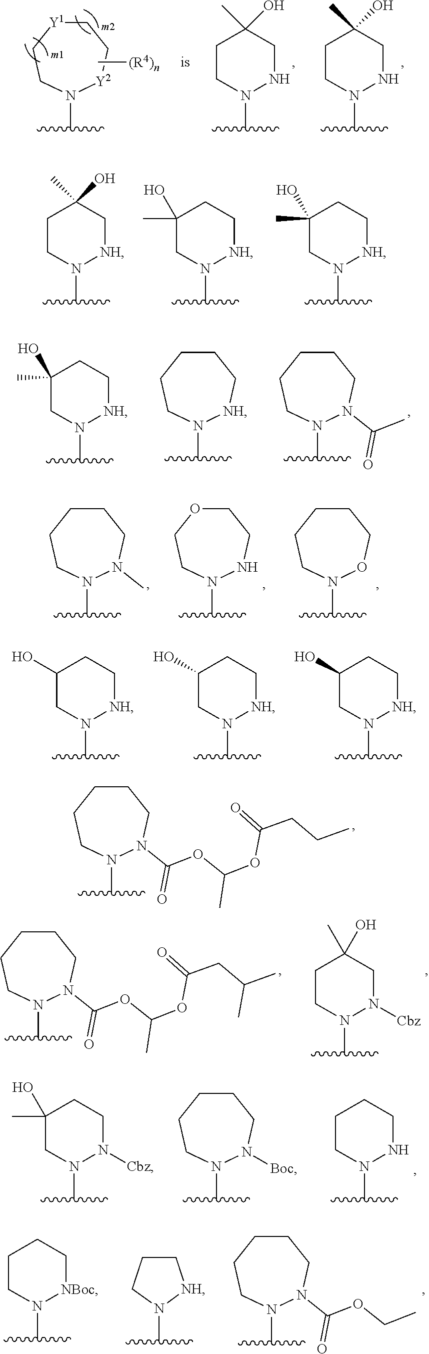

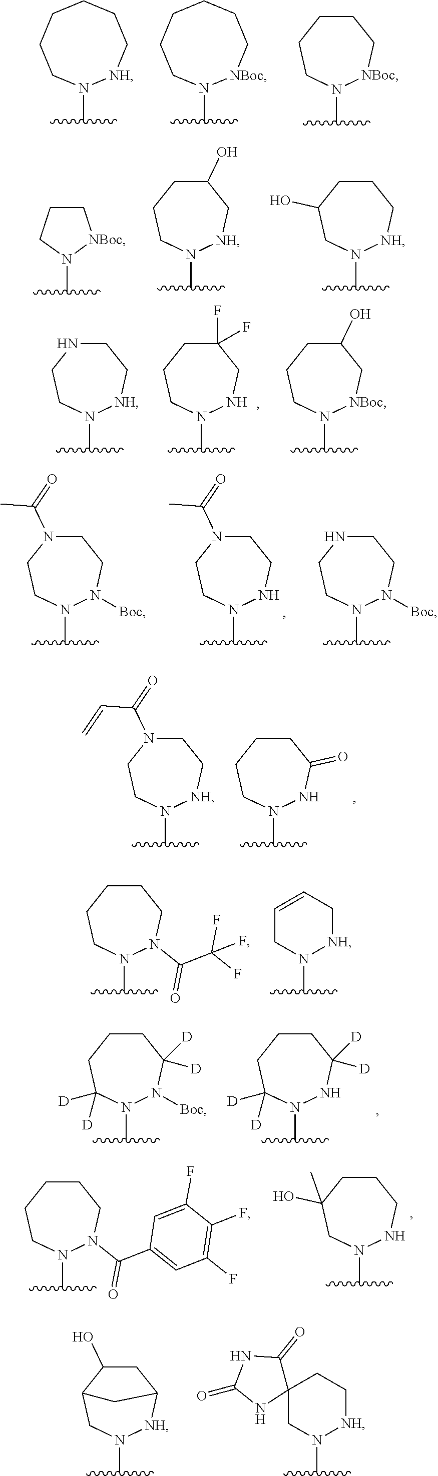

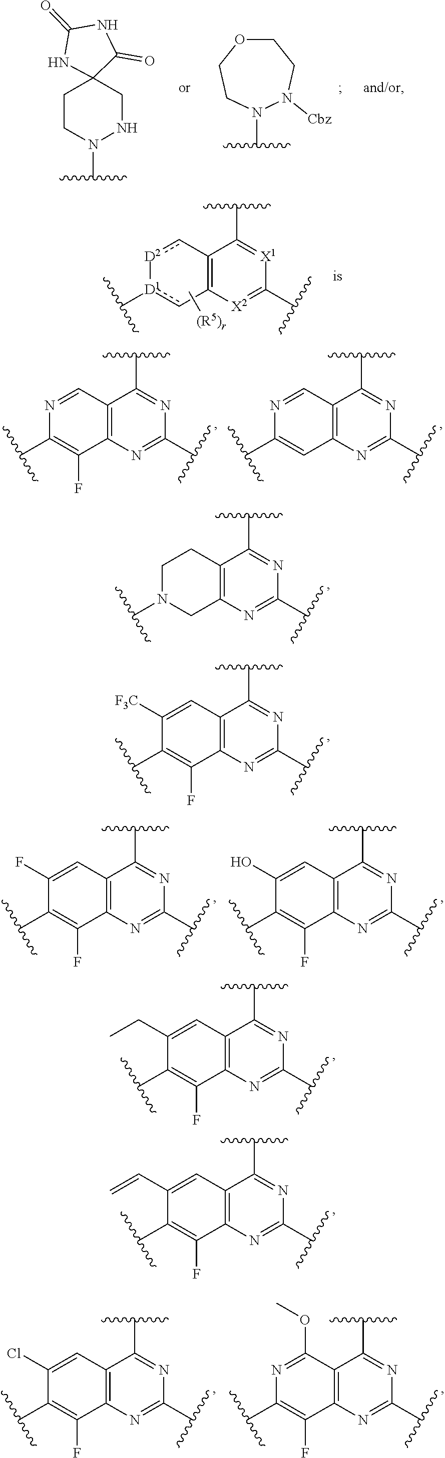

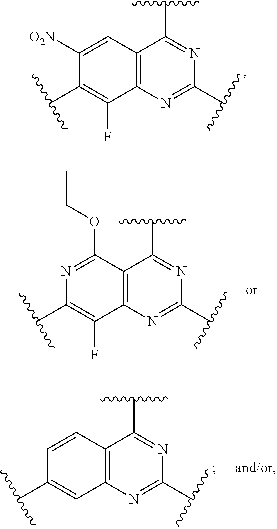 [SMILES: *c1nc(*)c2cc([N+](=O)[O-])c(*)c(F)c2n1 ; *c1nc(*)c2c(OCC)nc(*)c(F)c2n1 ; *c1ccc2c(*)nc(*)nc2c1]

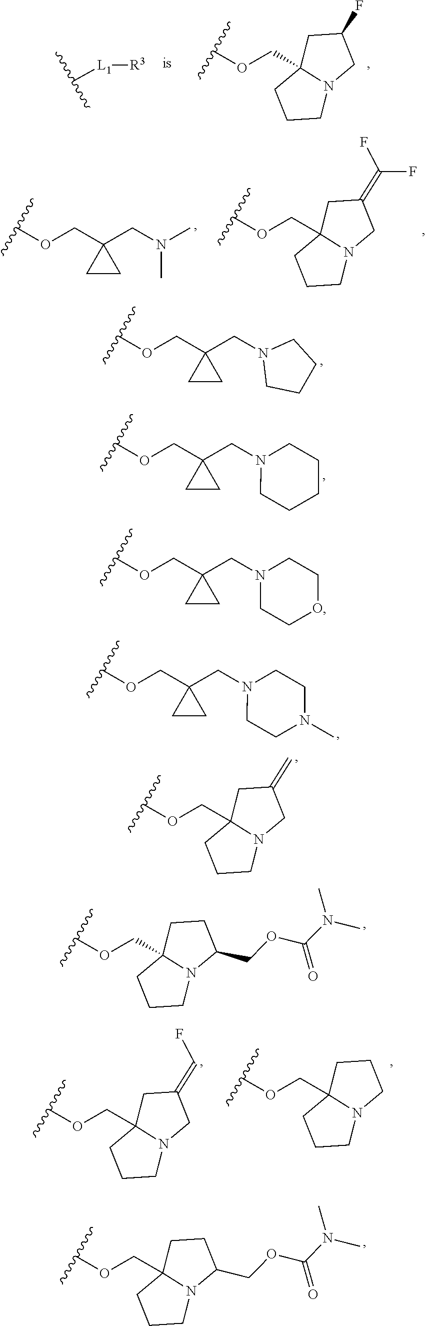

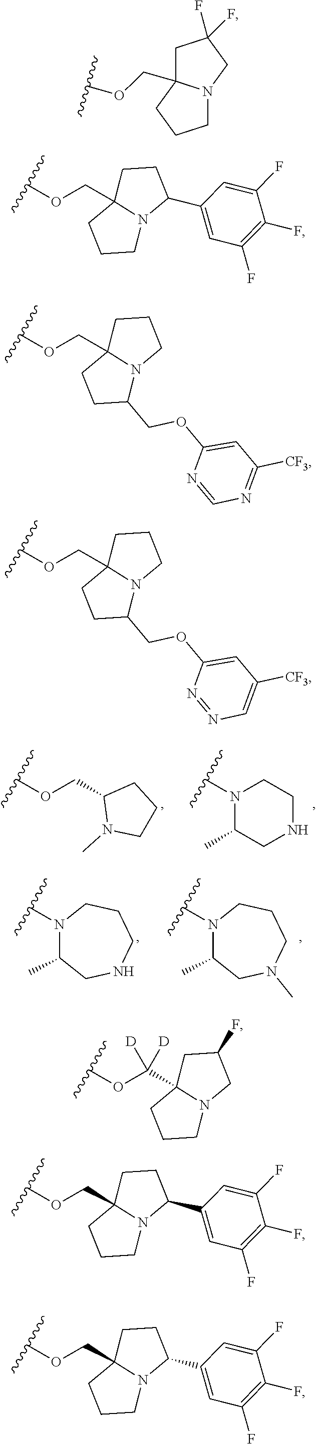

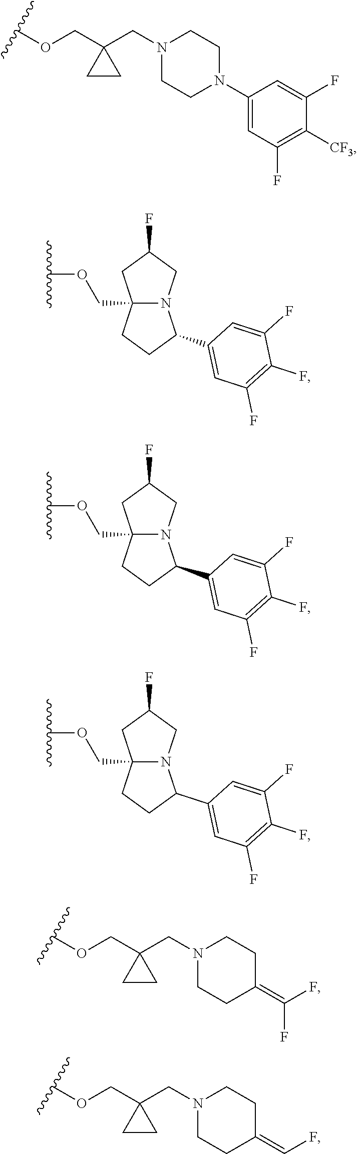 [SMILES: *OCC1(CN2CCN(c3cc(F)c(C(F)(F)F)c(F)c3)CC2)CC1 ; *OC[C@@]12CC[C@@H](c3cc(F)c(F)c(F)c3)N1C[C@H](F)C2 ; *OC[C@@]12CC[C@H](c3cc(F)c(F)c(F)c3)N1C[C@H](F)C2 ; *OC[C@@]12CCC(c3cc(F)c(F)c(F)c3)N1C[C@H](F)C2 ; *OCC1(CN2CCC(=C(F)F)CC2)CC1 ; *OCC1(CN2CCC(=CF)CC2)CC1]

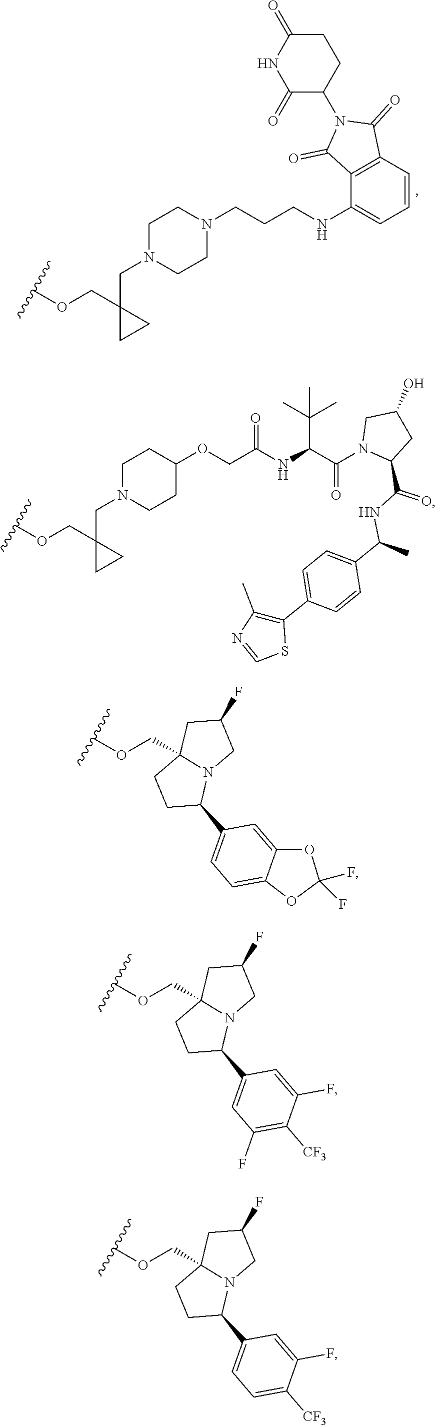

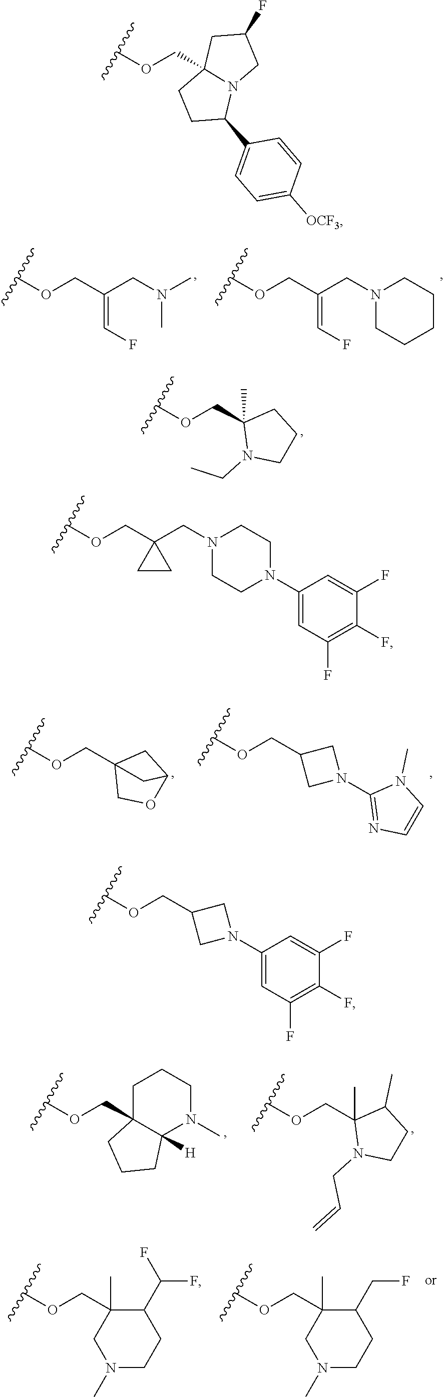

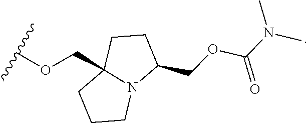 [SMILES: *OC[C@]12CCCN1[C@H](COC(=O)N(C)C)CC2]

8. The nitrogen-containing heterocyclic compound of formula I, the pharmaceutically acceptable salt thereof, a prodrug thereof, a stereoisomer thereof, a tautomer thereof or an isotopically labeled compound thereof according to claim 1, wherein,

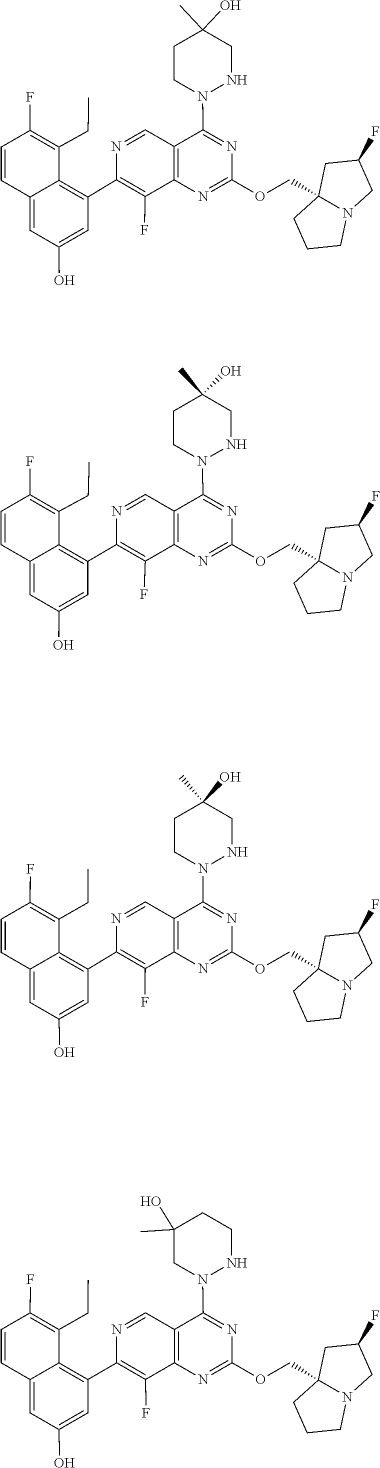 [SMILES: CCc1c(F)ccc2cc(O)cc(-c3ncc4c(N5CCC(C)(O)CN5)nc(OC[C@@]56CCCN5C[C@H](F)C6)nc4c3F)c12 ; CCc1c(F)ccc2cc(O)cc(-c3ncc4c(N5CC[C@](C)(O)CN5)nc(OC[C@@]56CCCN5C[C@H](F)C6)nc4c3F)c12 ; CCc1c(F)ccc2cc(O)cc(-c3ncc4c(N5CC[C@@](C)(O)CN5)nc(OC[C@@]56CCCN5C[C@H](F)C6)nc4c3F)c12 ; CCc1c(F)ccc2cc(O)cc(-c3ncc4c(N5CC(C)(O)CCN5)nc(OC[C@@]56CCCN5C[C@H](F)C6)nc4c3F)c12]

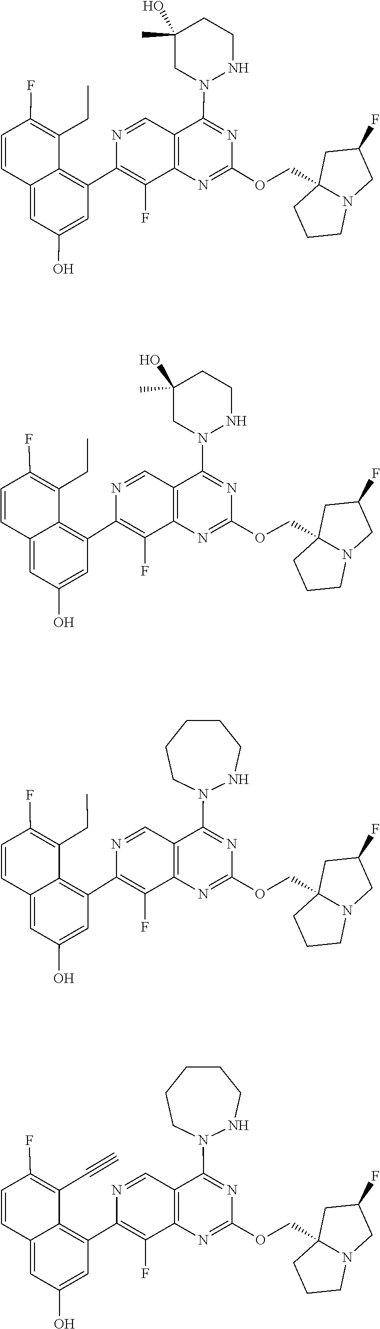 [SMILES: CCc1c(F)ccc2cc(O)cc(-c3ncc4c(N5C[C@](C)(O)CCN5)nc(OC[C@@]56CCCN5C[C@H](F)C6)nc4c3F)c12 ; CCc1c(F)ccc2cc(O)cc(-c3ncc4c(N5C[C@@](C)(O)CCN5)nc(OC[C@@]56CCCN5C[C@H](F)C6)nc4c3F)c12 ; CCc1c(F)ccc2cc(O)cc(-c3ncc4c(N5CCCCCN5)nc(OC[C@@]56CCCN5C[C@H](F)C6)nc4c3F)c12 ; C#Cc1c(F)ccc2cc(O)cc(-c3ncc4c(N5CCCCCN5)nc(OC[C@@]56CCCN5C[C@H](F)C6)nc4c3F)c12]

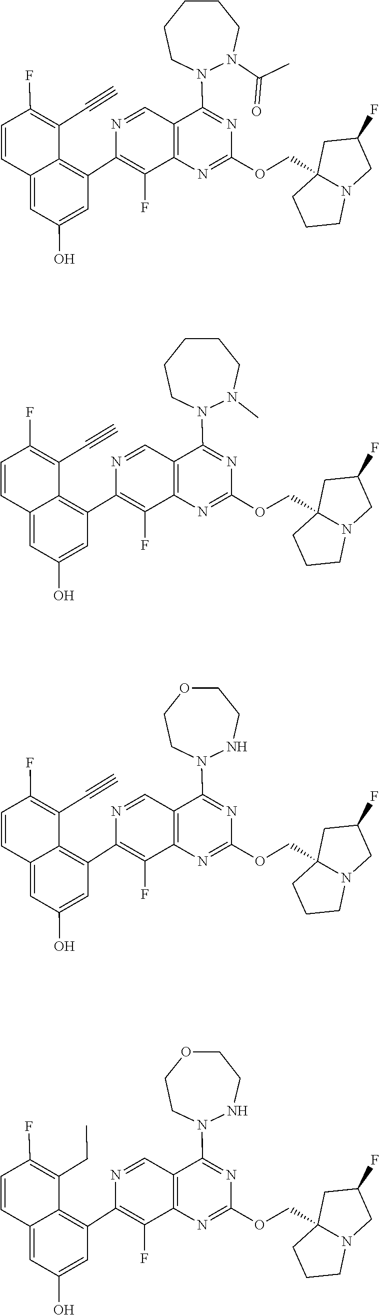 [SMILES: C#Cc1c(F)ccc2cc(O)cc(-c3ncc4c(N5CCCCCN5C(C)=O)nc(OC[C@@]56CCCN5C[C@H](F)C6)nc4c3F)c12 ; C#Cc1c(F)ccc2cc(O)cc(-c3ncc4c(N5CCCCCN5C)nc(OC[C@@]56CCCN5C[C@H](F)C6)nc4c3F)c12 ; C#Cc1c(F)ccc2cc(O)cc(-c3ncc4c(N5CCOCCN5)nc(OC[C@@]56CCCN5C[C@H](F)C6)nc4c3F)c12 ; CCc1c(F)ccc2cc(O)cc(-c3ncc4c(N5CCOCCN5)nc(OC[C@@]56CCCN5C[C@H](F)C6)nc4c3F)c12]

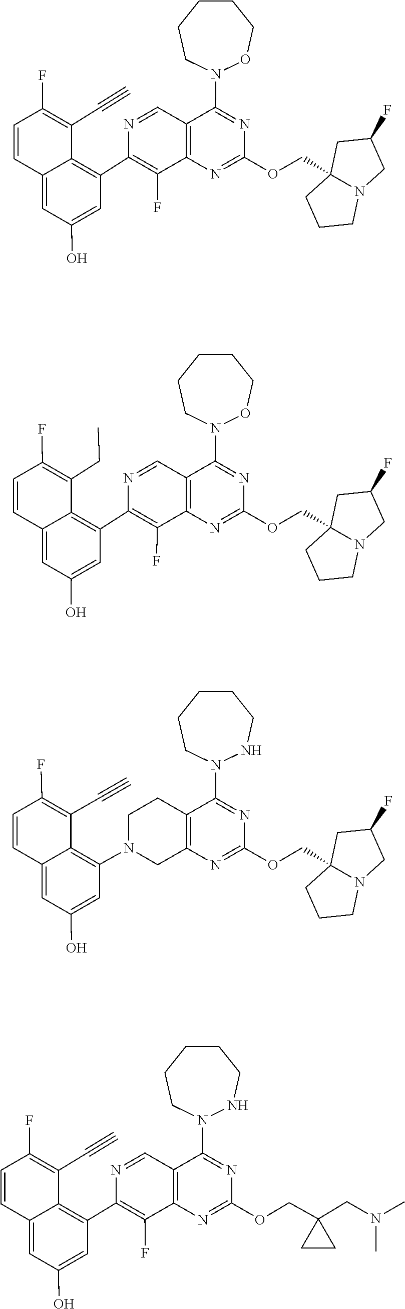 [SMILES: C#Cc1c(F)ccc2cc(O)cc(-c3ncc4c(N5CCCCCO5)nc(OC[C@@]56CCCN5C[C@H](F)C6)nc4c3F)c12 ; CCc1c(F)ccc2cc(O)cc(-c3ncc4c(N5CCCCCO5)nc(OC[C@@]56CCCN5C[C@H](F)C6)nc4c3F)c12 ; C#Cc1c(F)ccc2cc(O)cc(N3CCc4c(nc(OC[C@@]56CCCN5C[C@H](F)C6)nc4N4CCCCCN4)C3)c12 ; C#Cc1c(F)ccc2cc(O)cc(-c3ncc4c(N5CCCCCN5)nc(OCC5(CN(C)C)CC5)nc4c3F)c12]

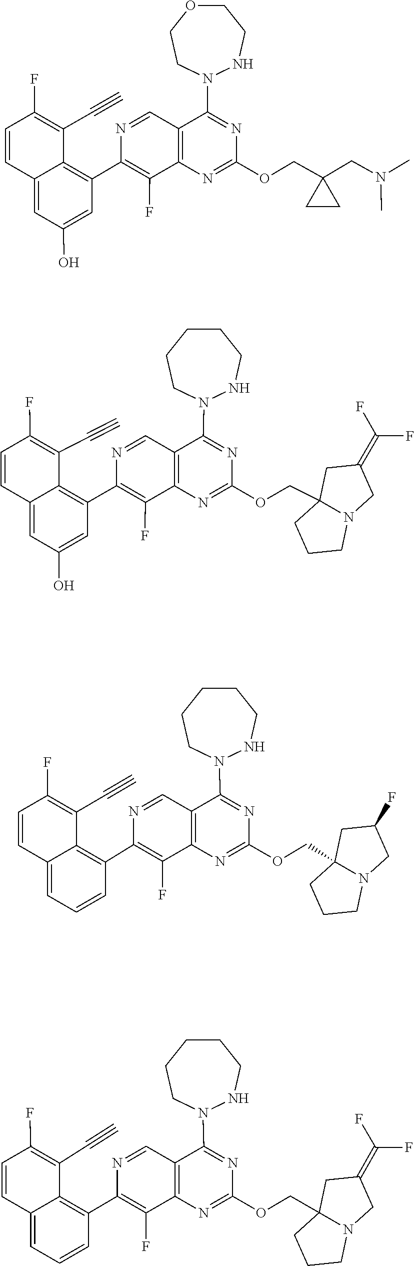 [SMILES: C#Cc1c(F)ccc2cc(O)cc(-c3ncc4c(N5CCOCCN5)nc(OCC5(CN(C)C)CC5)nc4c3F)c12 ; C#Cc1c(F)ccc2cc(O)cc(-c3ncc4c(N5CCCCCN5)nc(OCC56CCCN5CC(=C(F)F)C6)nc4c3F)c12 ; C#Cc1c(F)ccc2cccc(-c3ncc4c(N5CCCCCN5)nc(OC[C@@]56CCCN5C[C@H](F)C6)nc4c3F)c12 ; C#Cc1c(F)ccc2cccc(-c3ncc4c(N5CCCCCN5)nc(OCC56CCCN5CC(=C(F)F)C6)nc4c3F)c12]

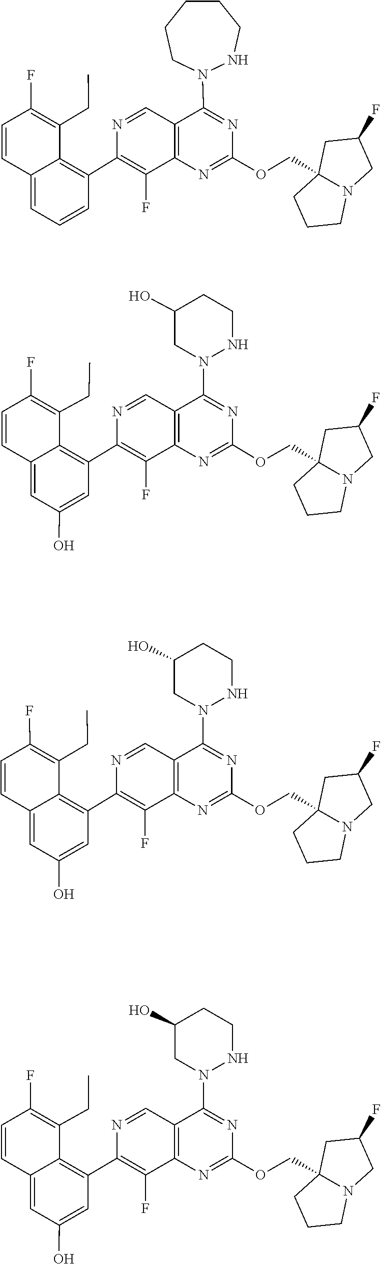 [SMILES: CCc1c(F)ccc2cccc(-c3ncc4c(N5CCCCCN5)nc(OC[C@@]56CCCN5C[C@H](F)C6)nc4c3F)c12 ; CCc1c(F)ccc2cc(O)cc(-c3ncc4c(N5CC(O)CCN5)nc(OC[C@@]56CCCN5C[C@H](F)C6)nc4c3F)c12 ; CCc1c(F)ccc2cc(O)cc(-c3ncc4c(N5C[C@H](O)CCN5)nc(OC[C@@]56CCCN5C[C@H](F)C6)nc4c3F)c12 ; CCc1c(F)ccc2cc(O)cc(-c3ncc4c(N5C[C@@H](O)CCN5)nc(OC[C@@]56CCCN5C[C@H](F)C6)nc4c3F)c12]

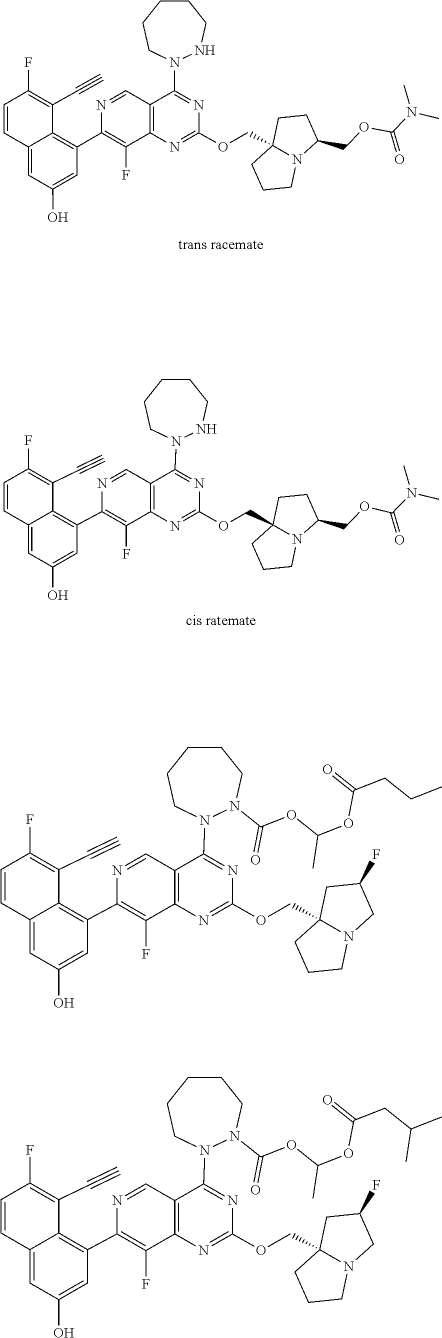 [SMILES: C#Cc1c(F)ccc2cc(O)cc(-c3ncc4c(N5CCCCCN5)nc(OC[C@@]56CCCN5[C@H](COC(=O)N(C)C)CC6)nc4c3F)c12 ; C#Cc1c(F)ccc2cc(O)cc(-c3ncc4c(N5CCCCCN5)nc(OC[C@]56CCCN5[C@H](COC(=O)N(C)C)CC6)nc4c3F)c12 ; C#Cc1c(F)ccc2cc(O)cc(-c3ncc4c(N5CCCCCN5C(=O)OC(C)OC(=O)CCC)nc(OC[C@@]56CCCN5C[C@H](F)C6)nc4c3F)c12 ; C#Cc1c(F)ccc2cc(O)cc(-c3ncc4c(N5CCCCCN5C(=O)OC(C)OC(=O)CC(C)C)nc(OC[C@@]56CCCN5C[C@H](F)C6)nc4c3F)c12]

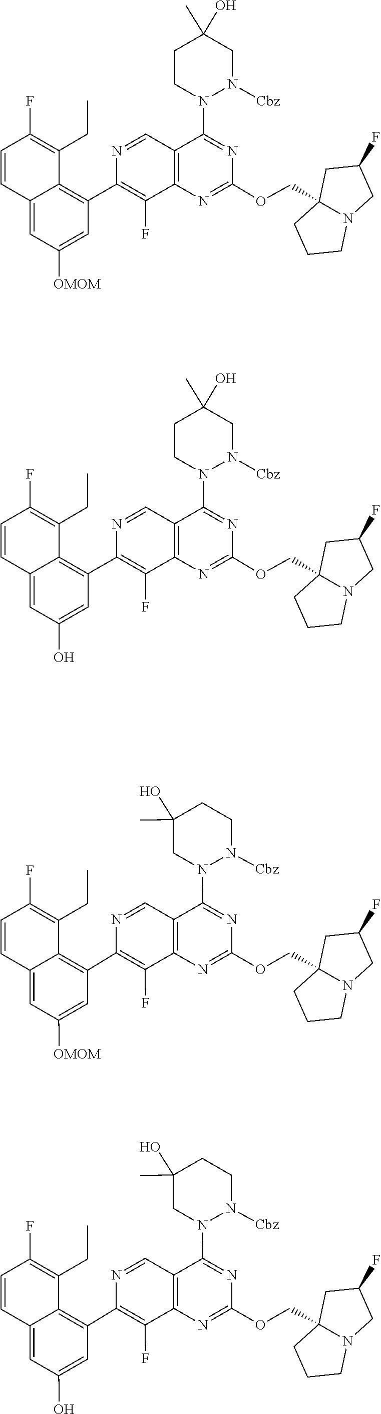

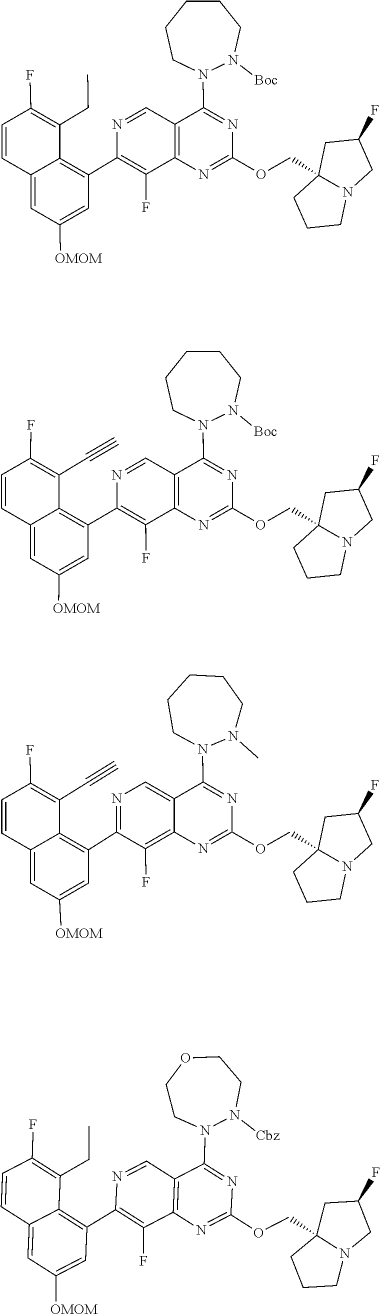

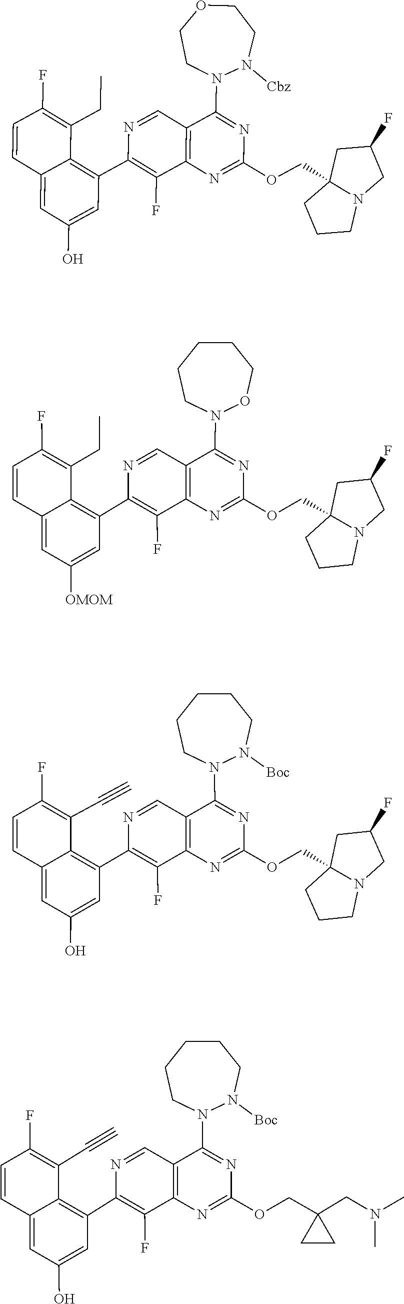

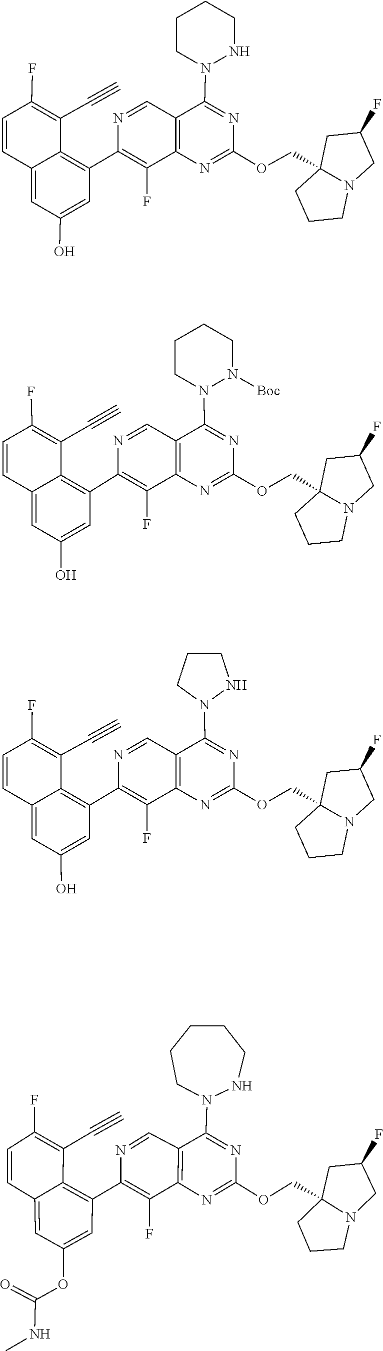 [SMILES: C#Cc1c(F)ccc2cc(O)cc(-c3ncc4c(N5CCCCN5)nc(OC[C@@]56CCCN5C[C@H](F)C6)nc4c3F)c12 ; C#Cc1c(F)ccc2cc(O)cc(-c3ncc4c(N5CCCCN5C(=O)OC(C)(C)C)nc(OC[C@@]56CCCN5C[C@H](F)C6)nc4c3F)c12 ; C#Cc1c(F)ccc2cc(O)cc(-c3ncc4c(N5CCCN5)nc(OC[C@@]56CCCN5C[C@H](F)C6)nc4c3F)c12 ; C#Cc1c(F)ccc2cc(OC(=O)NC)cc(-c3ncc4c(N5CCCCCN5)nc(OC[C@@]56CCCN5C[C@H](F)C6)nc4c3F)c12]

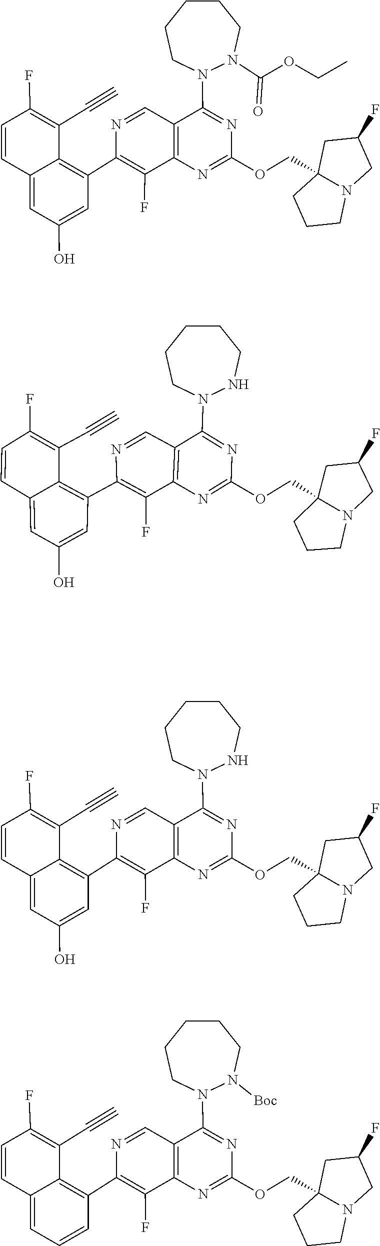 [SMILES: C#Cc1c(F)ccc2cc(O)cc(-c3ncc4c(N5CCCCCN5C(=O)OCC)nc(OC[C@@]56CCCN5C[C@H](F)C6)nc4c3F)c12 ; C#Cc1c(F)ccc2cc(O)cc(-c3ncc4c(N5CCCCCN5)nc(OC[C@@]56CCCN5C[C@H](F)C6)nc4c3F)c12 ; C#Cc1c(F)ccc2cc(O)cc(-c3ncc4c(N5CCCCCN5)nc(OC[C@@]56CCCN5C[C@H](F)C6)nc4c3F)c12 ; C#Cc1c(F)ccc2cccc(-c3ncc4c(N5CCCCCN5C(=O)OC(C)(C)C)nc(OC[C@@]56CCCN5C[C@H](F)C6)nc4c3F)c12]

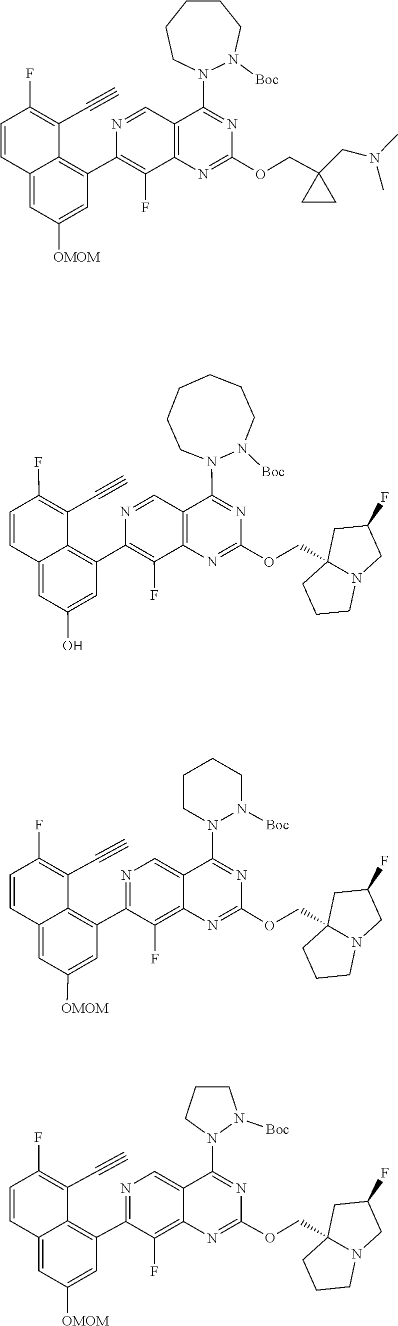

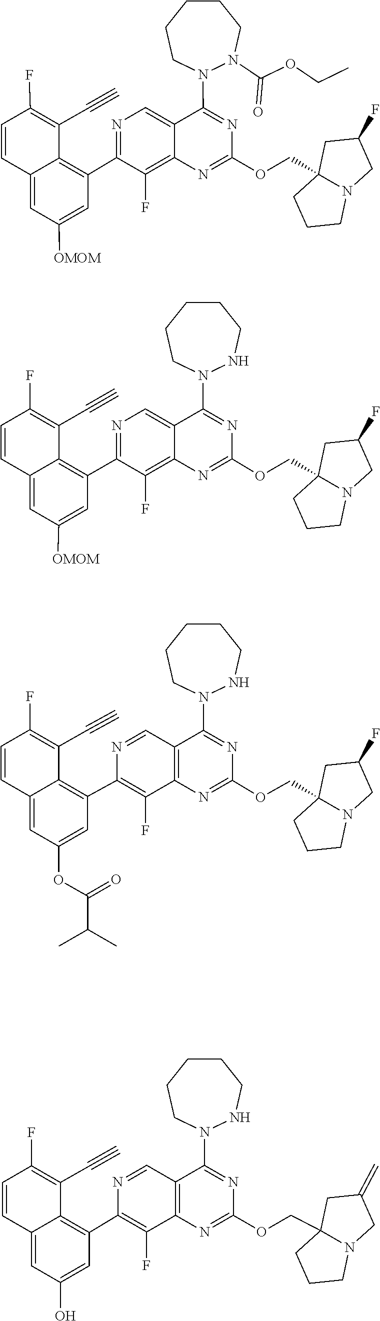

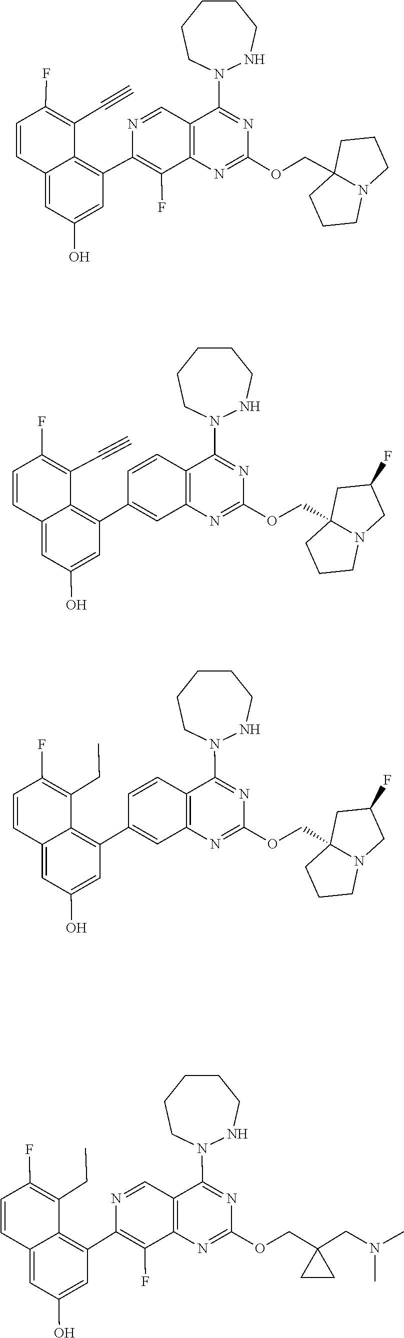 [SMILES: C#Cc1c(F)ccc2cc(O)cc(-c3ncc4c(N5CCCCCN5)nc(OCC56CCCN5CCC6)nc4c3F)c12 ; C#Cc1c(F)ccc2cc(O)cc(-c3ccc4c(N5CCCCCN5)nc(OC[C@@]56CCCN5C[C@H](F)C6)nc4c3)c12 ; CCc1c(F)ccc2cc(O)cc(-c3ccc4c(N5CCCCCN5)nc(OC[C@@]56CCCN5C[C@H](F)C6)nc4c3)c12 ; CCc1c(F)ccc2cc(O)cc(-c3ncc4c(N5CCCCCN5)nc(OCC5(CN(C)C)CC5)nc4c3F)c12]

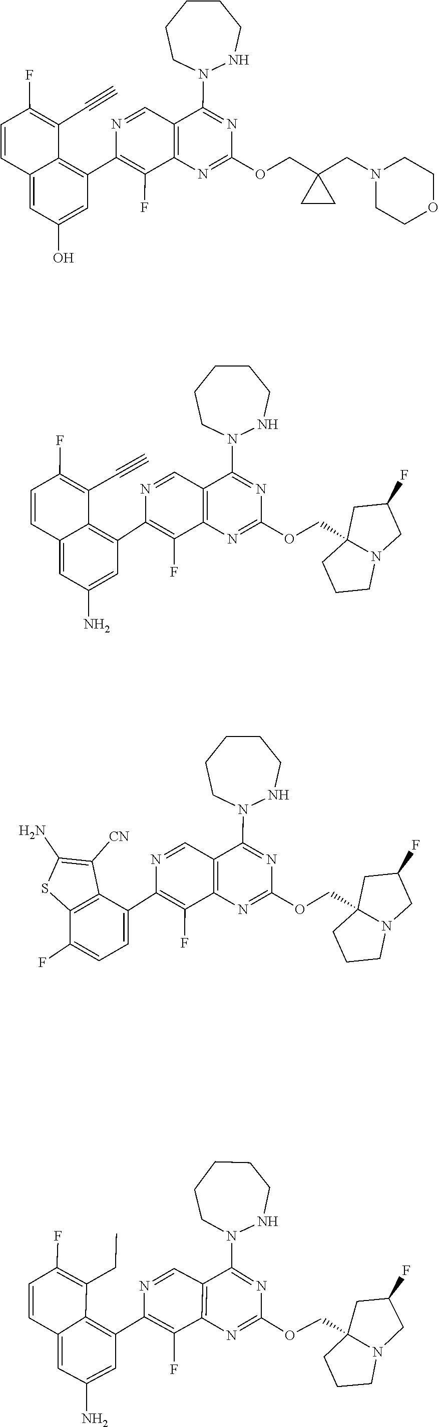

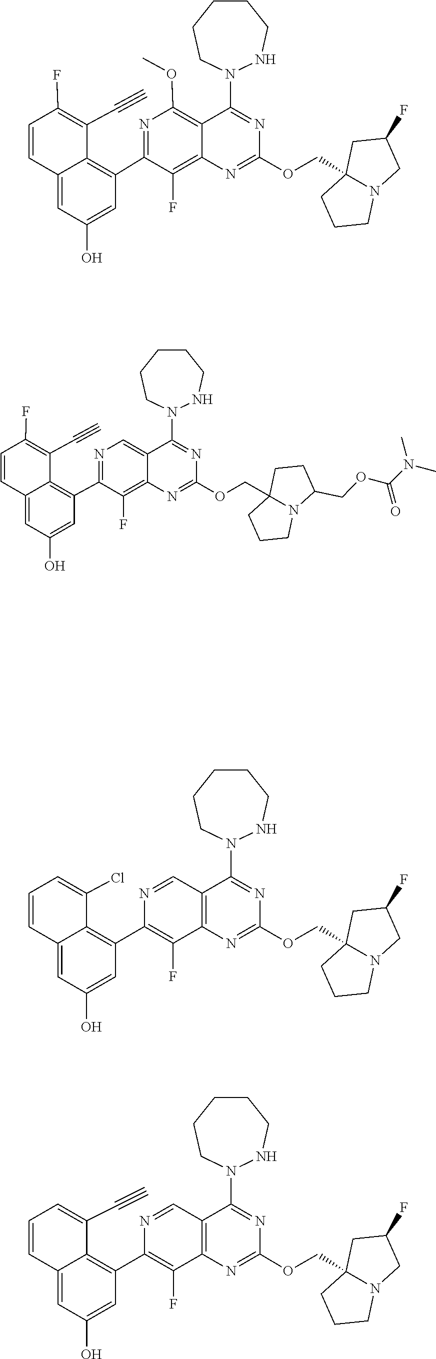 [SMILES: C#Cc1c(F)ccc2cc(O)cc(-c3nc(OC)c4c(N5CCCCCN5)nc(OC[C@@]56CCCN5C[C@H](F)C6)nc4c3F)c12 ; C#Cc1c(F)ccc2cc(O)cc(-c3ncc4c(N5CCCCCN5)nc(OCC56CCCN5C(COC(=O)N(C)C)CC6)nc4c3F)c12 ; Oc1cc(-c2ncc3c(N4CCCCCN4)nc(OC[C@@]45CCCN4C[C@H](F)C5)nc3c2F)c2c(Cl)cccc2c1 ; C#Cc1cccc2cc(O)cc(-c3ncc4c(N5CCCCCN5)nc(OC[C@@]56CCCN5C[C@H](F)C6)nc4c3F)c12]

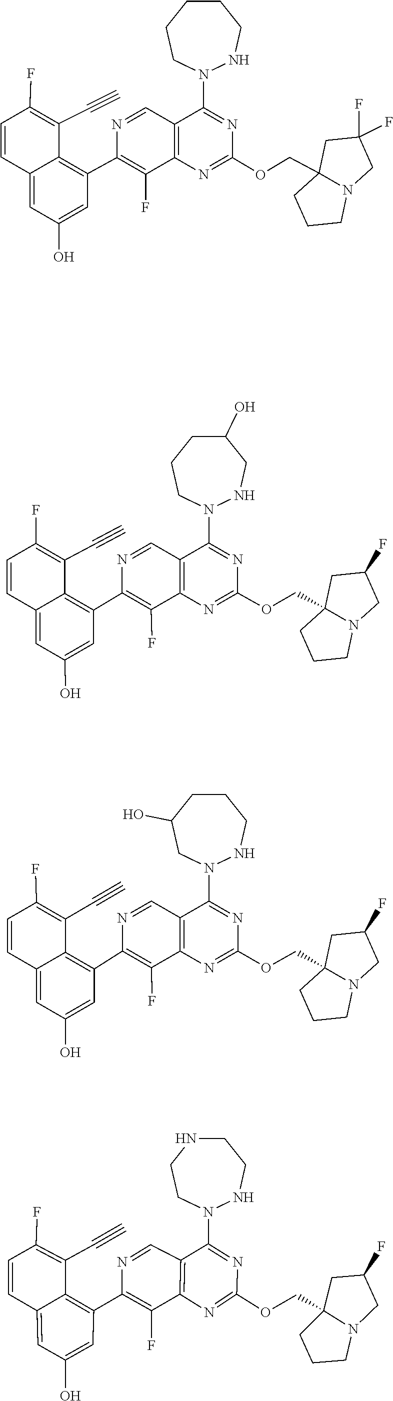 [SMILES: C#Cc1c(F)ccc2cc(O)cc(-c3ncc4c(N5CCCCCN5)nc(OCC56CCCN5CC(F)(F)C6)nc4c3F)c12 ; C#Cc1c(F)ccc2cc(O)cc(-c3ncc4c(N5CCCC(O)CN5)nc(OC[C@@]56CCCN5C[C@H](F)C6)nc4c3F)c12 ; C#Cc1c(F)ccc2cc(O)cc(-c3ncc4c(N5CC(O)CCCN5)nc(OC[C@@]56CCCN5C[C@H](F)C6)nc4c3F)c12 ; C#Cc1c(F)ccc2cc(O)cc(-c3ncc4c(N5CCNCCN5)nc(OC[C@@]56CCCN5C[C@H](F)C6)nc4c3F)c12]

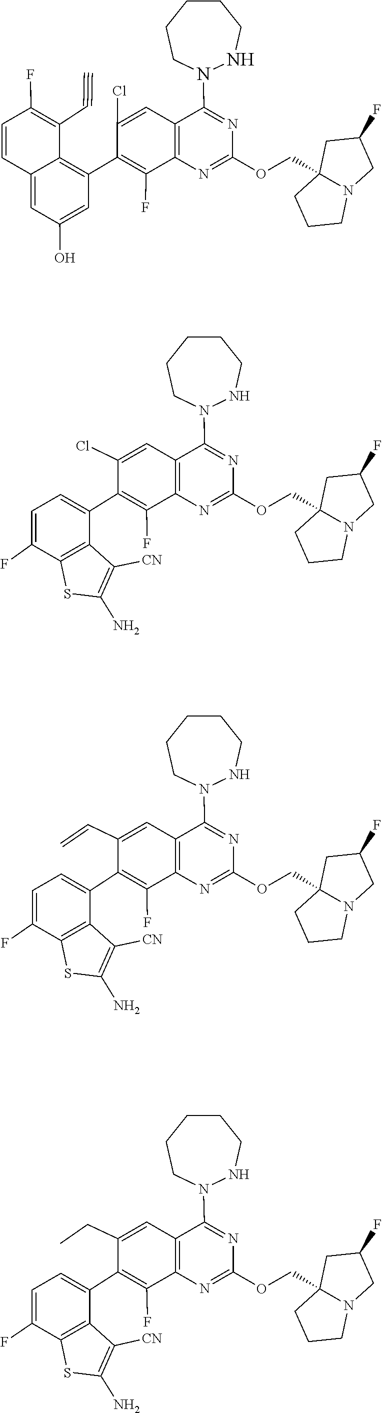 [SMILES: C#Cc1c(F)ccc2cc(O)cc(-c3c(Cl)cc4c(N5CCCCCN5)nc(OC[C@@]56CCCN5C[C@H](F)C6)nc4c3F)c12 ; N#Cc1c(N)sc2c(F)ccc(-c3c(Cl)cc4c(N5CCCCCN5)nc(OC[C@@]56CCCN5C[C@H](F)C6)nc4c3F)c12 ; C=Cc1cc2c(N3CCCCCN3)nc(OC[C@@]34CCCN3C[C@H](F)C4)nc2c(F)c1-c1ccc(F)c2sc(N)c(C#N)c12 ; CCc1cc2c(N3CCCCCN3)nc(OC[C@@]34CCCN3C[C@H](F)C4)nc2c(F)c1-c1ccc(F)c2sc(N)c(C#N)c12]

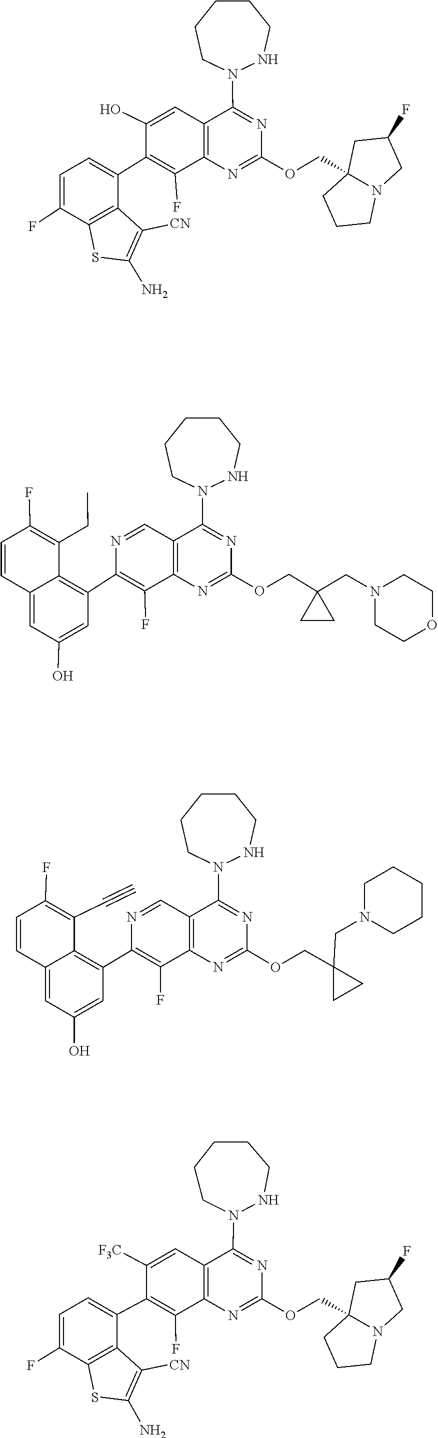 [SMILES: N#Cc1c(N)sc2c(F)ccc(-c3c(O)cc4c(N5CCCCCN5)nc(OC[C@@]56CCCN5C[C@H](F)C6)nc4c3F)c12 ; CCc1c(F)ccc2cc(O)cc(-c3ncc4c(N5CCCCCN5)nc(OCC5(CN6CCOCC6)CC5)nc4c3F)c12 ; C#Cc1c(F)ccc2cc(O)cc(-c3ncc4c(N5CCCCCN5)nc(OCC5(CN6CCCCC6)CC5)nc4c3F)c12 ; N#Cc1c(N)sc2c(F)ccc(-c3c(C(F)(F)F)cc4c(N5CCCCCN5)nc(OC[C@@]56CCCN5C[C@H](F)C6)nc4c3F)c12]

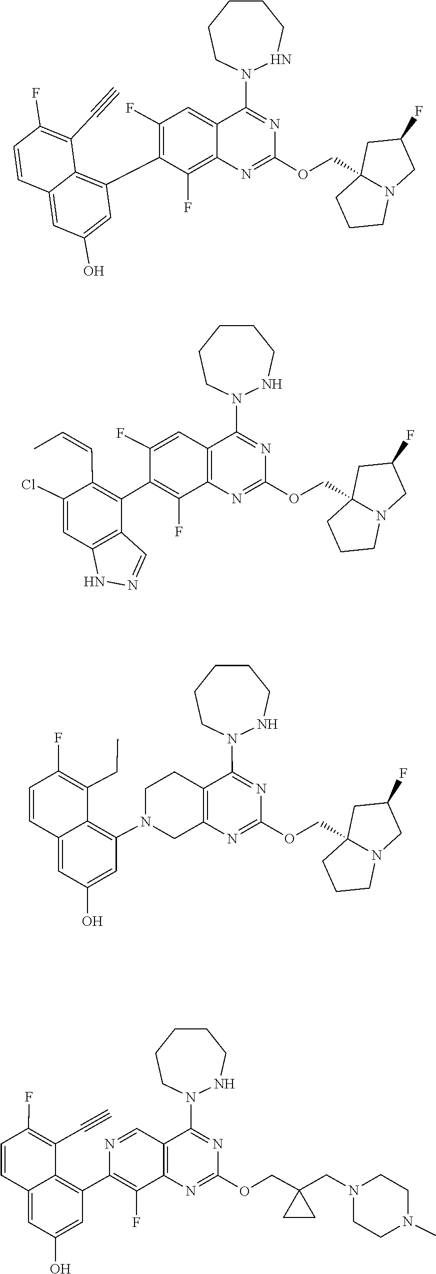 [SMILES: C#Cc1c(F)ccc2cc(O)cc(-c3c(F)cc4c(N5CCCCCN5)nc(OC[C@@]56CCCN5C[C@H](F)C6)nc4c3F)c12 ; C/C=C\c1c(Cl)cc2[nH]ncc2c1-c1c(F)cc2c(N3CCCCCN3)nc(OC[C@@]34CCCN3C[C@H](F)C4)nc2c1F ; CCc1c(F)ccc2cc(O)cc(N3CCc4c(nc(OC[C@@]56CCCN5C[C@H](F)C6)nc4N4CCCCCN4)C3)c12 ; C#Cc1c(F)ccc2cc(O)cc(-c3ncc4c(N5CCCCCN5)nc(OCC5(CN6CCN(C)CC6)CC5)nc4c3F)c12]

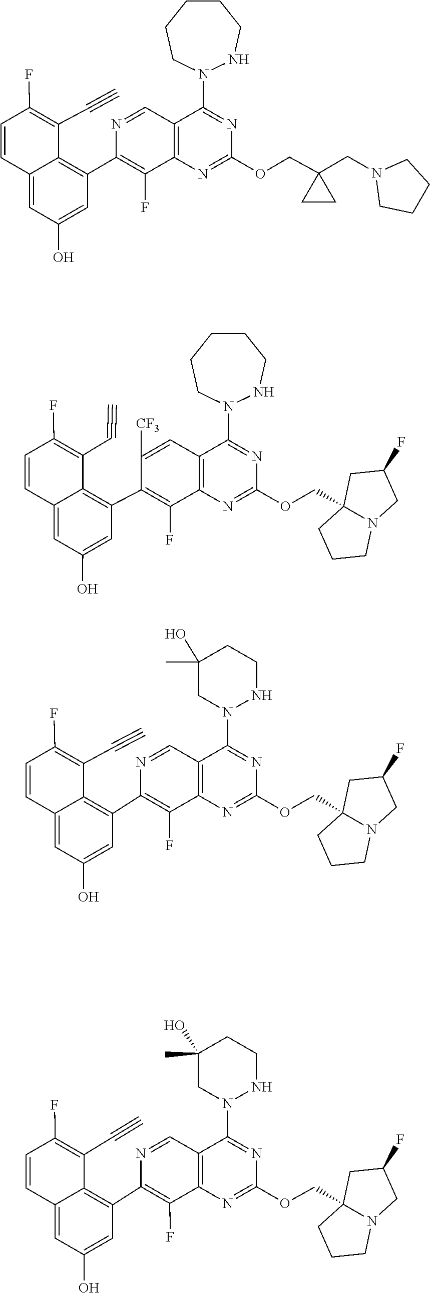 [SMILES: C#Cc1c(F)ccc2cc(O)cc(-c3ncc4c(N5CCCCCN5)nc(OCC5(CN6CCCC6)CC5)nc4c3F)c12 ; C#Cc1c(F)ccc2cc(O)cc(-c3c(C(F)(F)F)cc4c(N5CCCCCN5)nc(OC[C@@]56CCCN5C[C@H](F)C6)nc4c3F)c12 ; C#Cc1c(F)ccc2cc(O)cc(-c3ncc4c(N5CC(C)(O)CCN5)nc(OC[C@@]56CCCN5C[C@H](F)C6)nc4c3F)c12 ; C#Cc1c(F)ccc2cc(O)cc(-c3ncc4c(N5C[C@](C)(O)CCN5)nc(OC[C@@]56CCCN5C[C@H](F)C6)nc4c3F)c12]

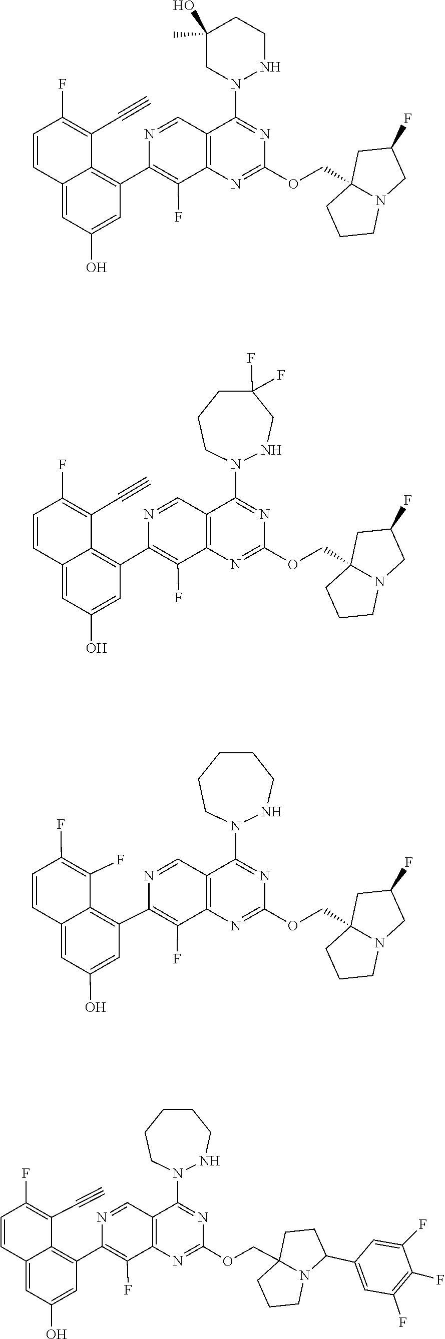 [SMILES: C#Cc1c(F)ccc2cc(O)cc(-c3ncc4c(N5C[C@@](C)(O)CCN5)nc(OC[C@@]56CCCN5C[C@H](F)C6)nc4c3F)c12 ; C#Cc1c(F)ccc2cc(O)cc(-c3ncc4c(N5CCCC(F)(F)CN5)nc(OC[C@@]56CCCN5C[C@H](F)C6)nc4c3F)c12 ; Oc1cc(-c2ncc3c(N4CCCCCN4)nc(OC[C@@]45CCCN4C[C@H](F)C5)nc3c2F)c2c(F)c(F)ccc2c1 ; C#Cc1c(F)ccc2cc(O)cc(-c3ncc4c(N5CCCCCN5)nc(OCC56CCCN5C(c5cc(F)c(F)c(F)c5)CC6)nc4c3F)c12]

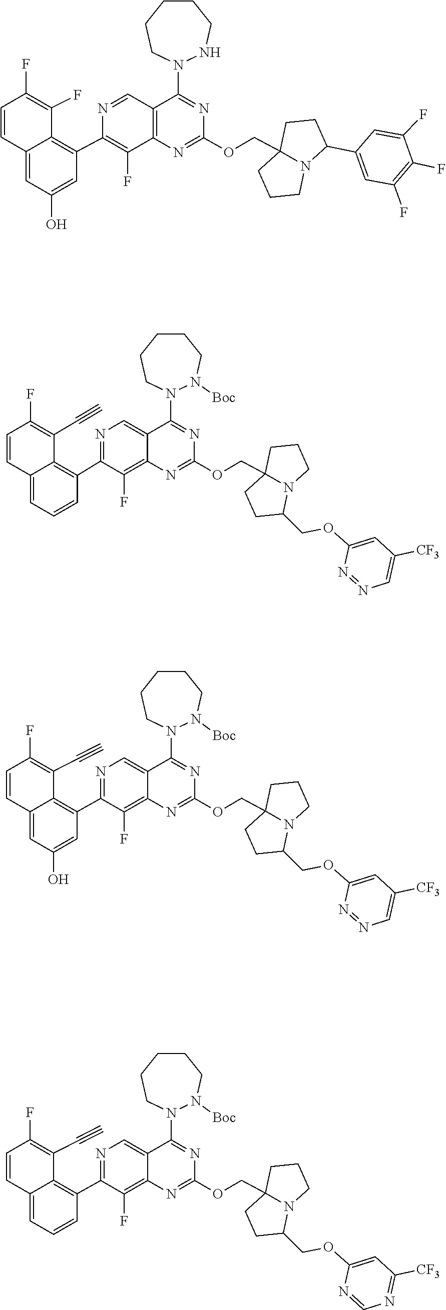 [SMILES: Oc1cc(-c2ncc3c(N4CCCCCN4)nc(OCC45CCCN4C(c4cc(F)c(F)c(F)c4)CC5)nc3c2F)c2c(F)c(F)ccc2c1 ; C#Cc1c(F)ccc2cccc(-c3ncc4c(N5CCCCCN5C(=O)OC(C)(C)C)nc(OCC56CCCN5C(COc5cc(C(F)(F)F)cnn5)CC6)nc4c3F)c12 ; C#Cc1c(F)ccc2cc(O)cc(-c3ncc4c(N5CCCCCN5C(=O)OC(C)(C)C)nc(OCC56CCCN5C(COc5cc(C(F)(F)F)cnn5)CC6)nc4c3F)c12 ; C#Cc1c(F)ccc2cccc(-c3ncc4c(N5CCCCCN5C(=O)OC(C)(C)C)nc(OCC56CCCN5C(COc5cc(C(F)(F)F)ncn5)CC6)nc4c3F)c12]

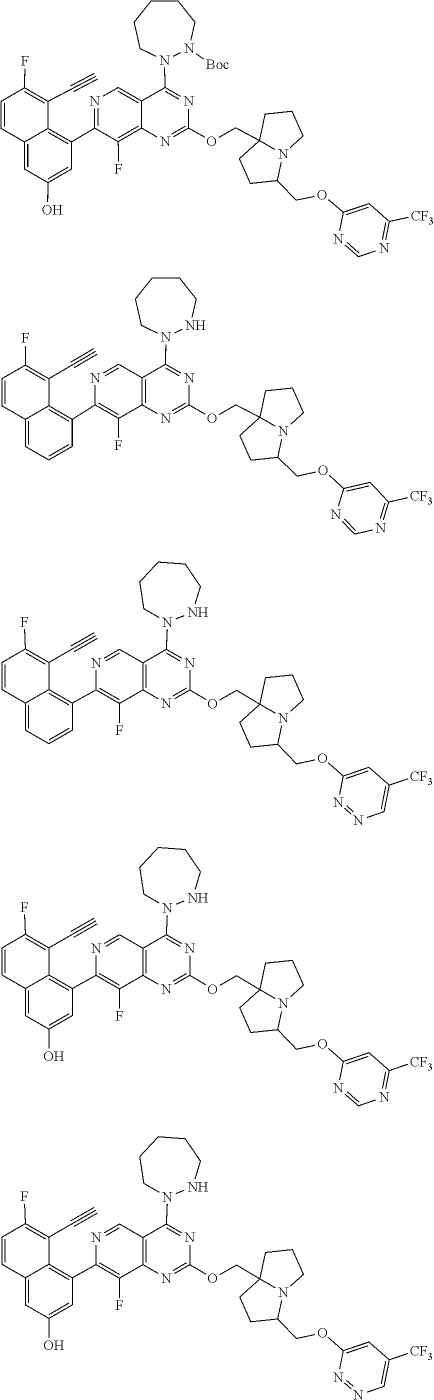 [SMILES: C#Cc1c(F)ccc2cc(O)cc(-c3ncc4c(N5CCCCCN5C(=O)OC(C)(C)C)nc(OCC56CCCN5C(COc5cc(C(F)(F)F)ncn5)CC6)nc4c3F)c12 ; C#Cc1c(F)ccc2cccc(-c3ncc4c(N5CCCCCN5)nc(OCC56CCCN5C(COc5cc(C(F)(F)F)ncn5)CC6)nc4c3F)c12 ; C#Cc1c(F)ccc2cccc(-c3ncc4c(N5CCCCCN5)nc(OCC56CCCN5C(COc5cc(C(F)(F)F)cnn5)CC6)nc4c3F)c12 ; C#Cc1c(F)ccc2cc(O)cc(-c3ncc4c(N5CCCCCN5)nc(OCC56CCCN5C(COc5cc(C(F)(F)F)ncn5)CC6)nc4c3F)c12 ; C#Cc1c(F)ccc2cc(O)cc(-c3ncc4c(N5CCCCCN5)nc(OCC56CCCN5C(COc5cc(C(F)(F)F)cnn5)CC6)nc4c3F)c12]

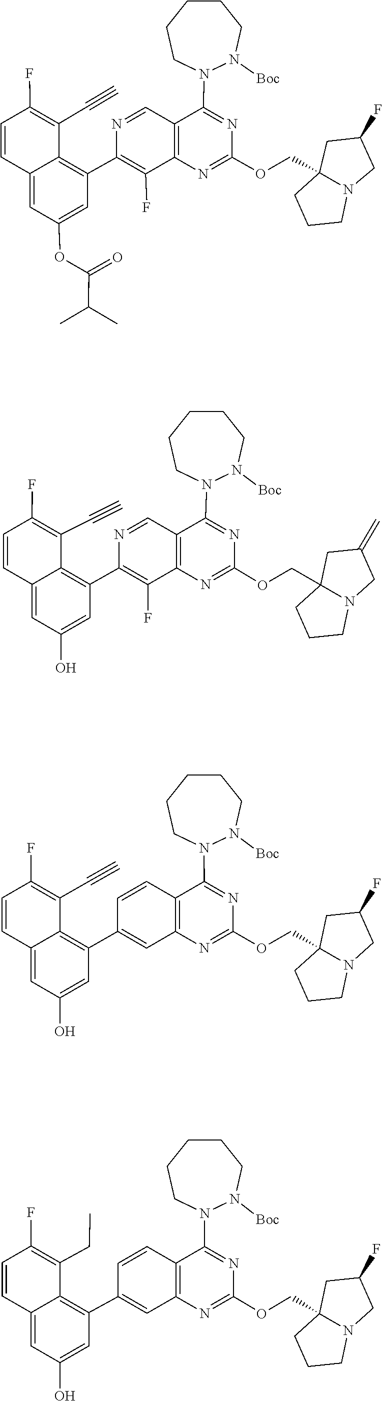 [SMILES: C#Cc1c(F)ccc2cc(OC(=O)C(C)C)cc(-c3ncc4c(N5CCCCCN5C(=O)OC(C)(C)C)nc(OC[C@@]56CCCN5C[C@H](F)C6)nc4c3F)c12 ; C#Cc1c(F)ccc2cc(O)cc(-c3ncc4c(N5CCCCCN5C(=O)OC(C)(C)C)nc(OCC56CCCN5CC(=C)C6)nc4c3F)c12 ; C#Cc1c(F)ccc2cc(O)cc(-c3ccc4c(N5CCCCCN5C(=O)OC(C)(C)C)nc(OC[C@@]56CCCN5C[C@H](F)C6)nc4c3)c12 ; CCc1c(F)ccc2cc(O)cc(-c3ccc4c(N5CCCCCN5C(=O)OC(C)(C)C)nc(OC[C@@]56CCCN5C[C@H](F)C6)nc4c3)c12]

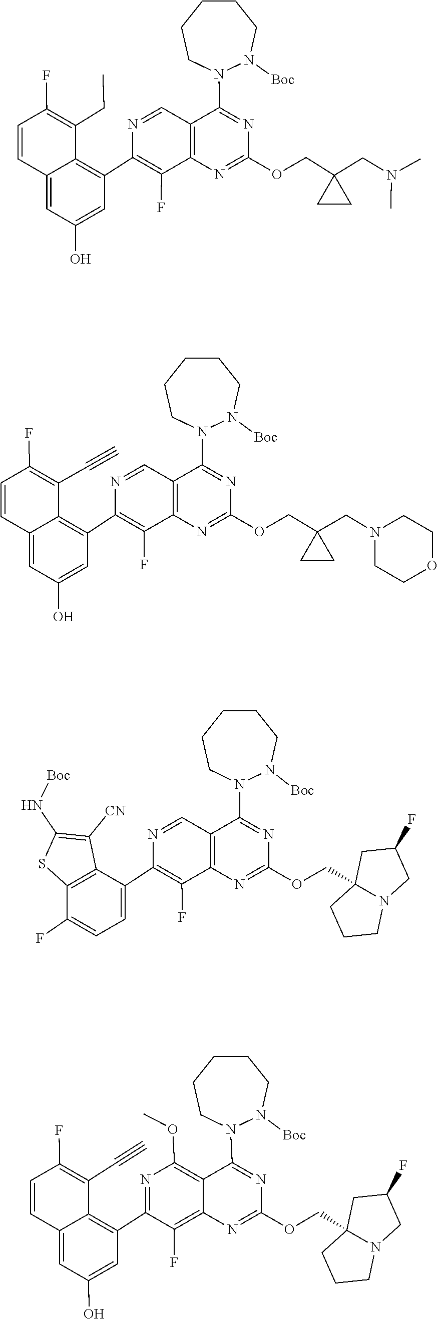 [SMILES: CCc1c(F)ccc2cc(O)cc(-c3ncc4c(N5CCCCCN5C(=O)OC(C)(C)C)nc(OCC5(CN(C)C)CC5)nc4c3F)c12 ; C#Cc1c(F)ccc2cc(O)cc(-c3ncc4c(N5CCCCCN5C(=O)OC(C)(C)C)nc(OCC5(CN6CCOCC6)CC5)nc4c3F)c12 ; CC(C)(C)OC(=O)Nc1sc2c(F)ccc(-c3ncc4c(N5CCCCCN5C(=O)OC(C)(C)C)nc(OC[C@@]56CCCN5C[C@H](F)C6)nc4c3F)c2c1C#N ; C#Cc1c(F)ccc2cc(O)cc(-c3nc(OC)c4c(N5CCCCCN5C(=O)OC(C)(C)C)nc(OC[C@@]56CCCN5C[C@H](F)C6)nc4c3F)c12]

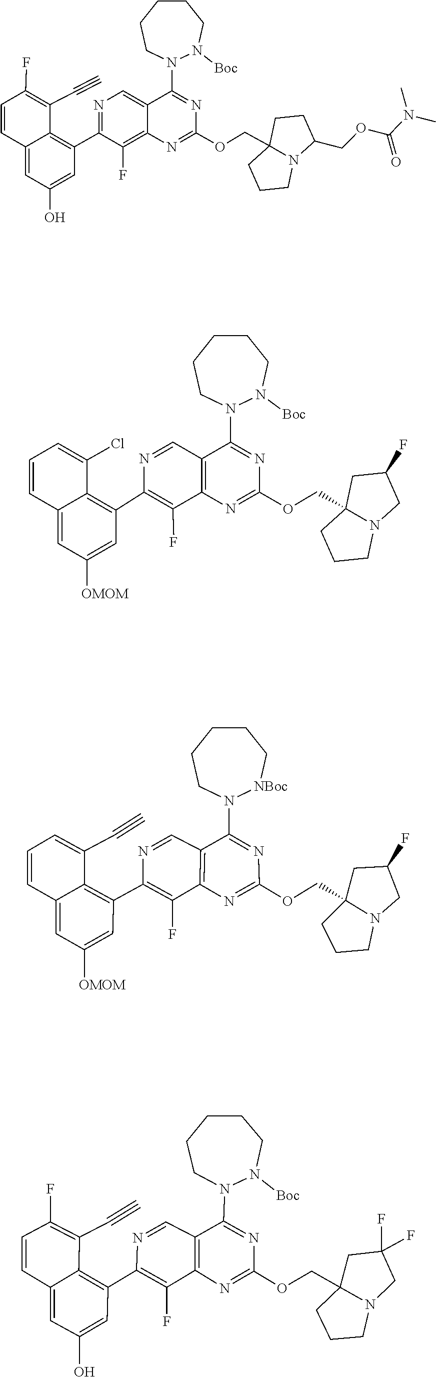

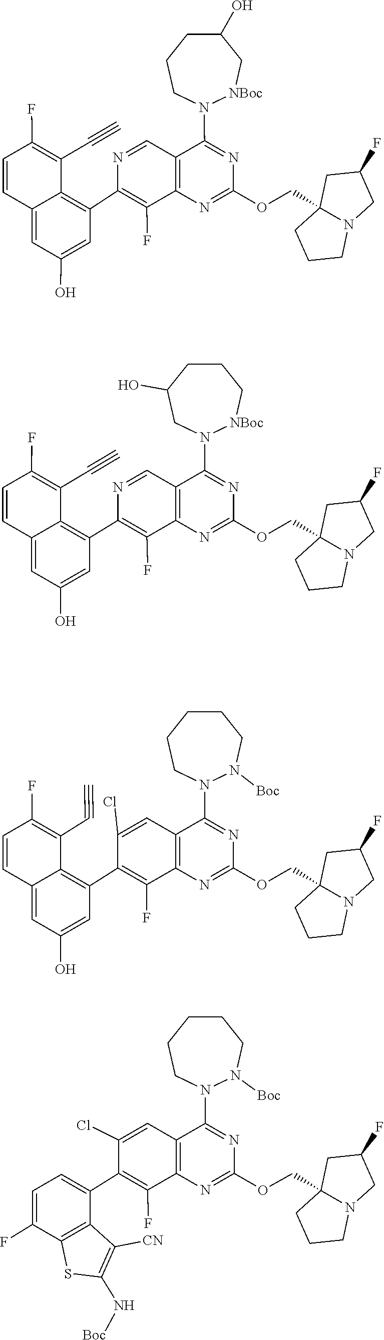 [SMILES: C#Cc1c(F)ccc2cc(O)cc(-c3ncc4c(N5CCCC(O)CN5C(=O)OC(C)(C)C)nc(OC[C@@]56CCCN5C[C@H](F)C6)nc4c3F)c12 ; C#Cc1c(F)ccc2cc(O)cc(-c3ncc4c(N5CC(O)CCCN5C(=O)OC(C)(C)C)nc(OC[C@@]56CCCN5C[C@H](F)C6)nc4c3F)c12 ; C#Cc1c(F)ccc2cc(O)cc(-c3c(Cl)cc4c(N5CCCCCN5C(=O)OC(C)(C)C)nc(OC[C@@]56CCCN5C[C@H](F)C6)nc4c3F)c12 ; CC(C)(C)OC(=O)Nc1sc2c(F)ccc(-c3c(Cl)cc4c(N5CCCCCN5C(=O)OC(C)(C)C)nc(OC[C@@]56CCCN5C[C@H](F)C6)nc4c3F)c2c1C#N]

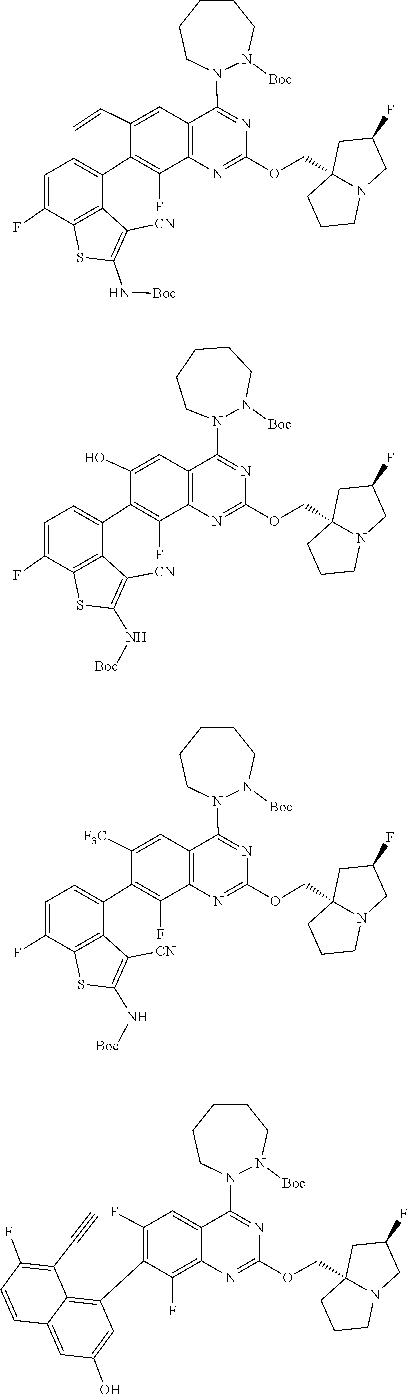 [SMILES: C=Cc1cc2c(N3CCCCCN3C(=O)OC(C)(C)C)nc(OC[C@@]34CCCN3C[C@H](F)C4)nc2c(F)c1-c1ccc(F)c2sc(NC(=O)OC(C)(C)C)c(C#N)c12 ; CC(C)(C)OC(=O)Nc1sc2c(F)ccc(-c3c(O)cc4c(N5CCCCCN5C(=O)OC(C)(C)C)nc(OC[C@@]56CCCN5C[C@H](F)C6)nc4c3F)c2c1C#N ; CC(C)(C)OC(=O)Nc1sc2c(F)ccc(-c3c(C(F)(F)F)cc4c(N5CCCCCN5C(=O)OC(C)(C)C)nc(OC[C@@]56CCCN5C[C@H](F)C6)nc4c3F)c2c1C#N ; C#Cc1c(F)ccc2cc(O)cc(-c3c(F)cc4c(N5CCCCCN5C(=O)OC(C)(C)C)nc(OC[C@@]56CCCN5C[C@H](F)C6)nc4c3F)c12]

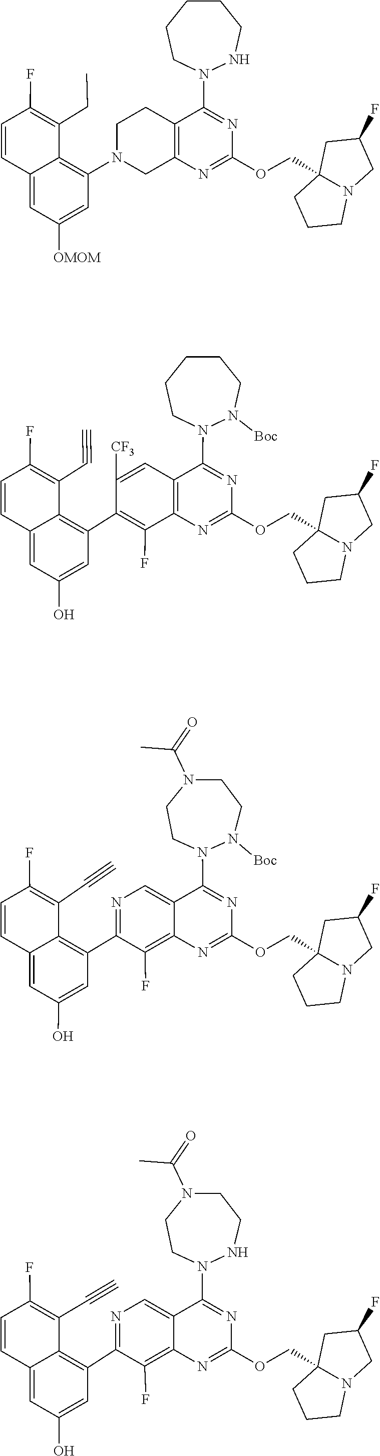

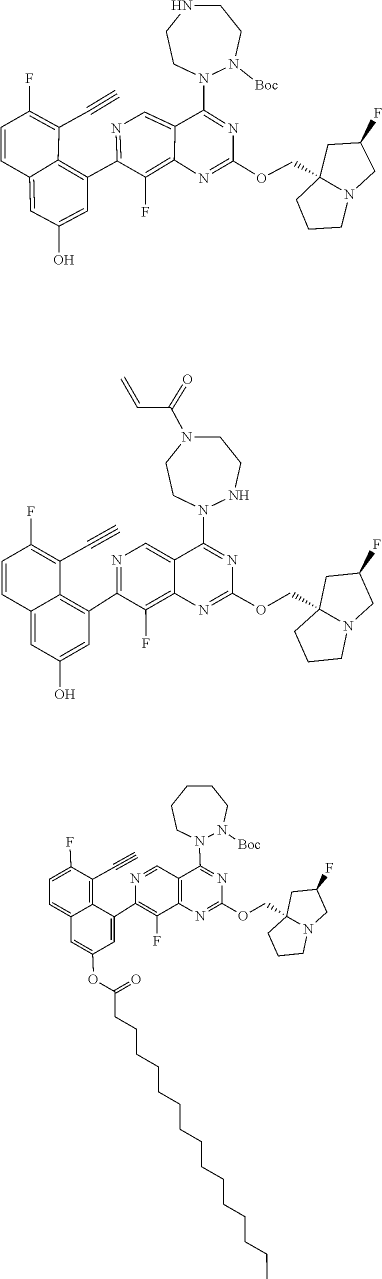 [SMILES: C#Cc1c(F)ccc2cc(O)cc(-c3ncc4c(N5CCNCCN5C(=O)OC(C)(C)C)nc(OC[C@@]56CCCN5C[C@H](F)C6)nc4c3F)c12 ; C#Cc1c(F)ccc2cc(O)cc(-c3ncc4c(N5CCN(C(=O)C=C)CCN5)nc(OC[C@@]56CCCN5C[C@H](F)C6)nc4c3F)c12 ; C#Cc1c(F)ccc2cc(OC(=O)CCCCCCCCCCCCCCC)cc(-c3ncc4c(N5CCCCCN5C(=O)OC(C)(C)C)nc(OC[C@@]56CCCN5C[C@H](F)C6)nc4c3F)c12]

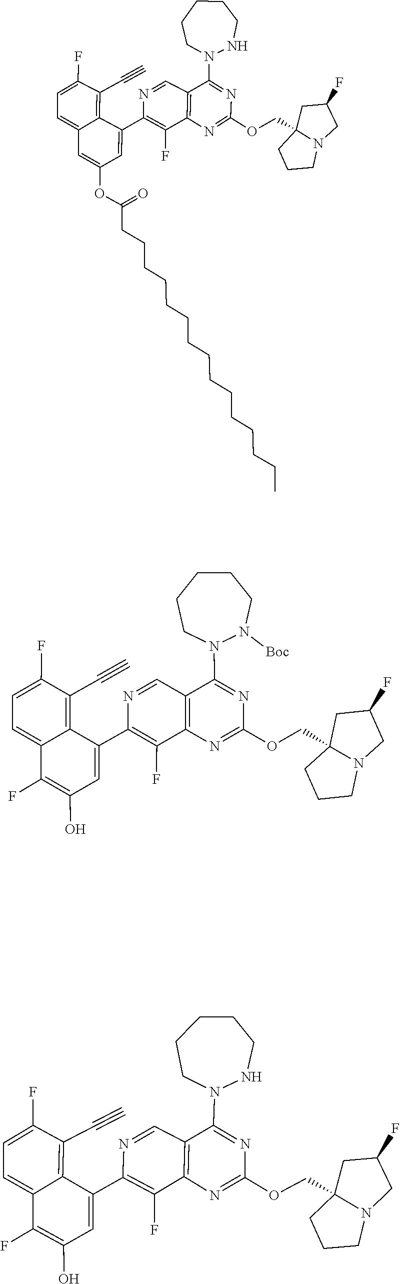 [SMILES: C#Cc1c(F)ccc2cc(OC(=O)CCCCCCCCCCCCCCC)cc(-c3ncc4c(N5CCCCCN5)nc(OC[C@@]56CCCN5C[C@H](F)C6)nc4c3F)c12 ; C#Cc1c(F)ccc2c(F)c(O)cc(-c3ncc4c(N5CCCCCN5C(=O)OC(C)(C)C)nc(OC[C@@]56CCCN5C[C@H](F)C6)nc4c3F)c12 ; C#Cc1c(F)ccc2c(F)c(O)cc(-c3ncc4c(N5CCCCCN5)nc(OC[C@@]56CCCN5C[C@H](F)C6)nc4c3F)c12]

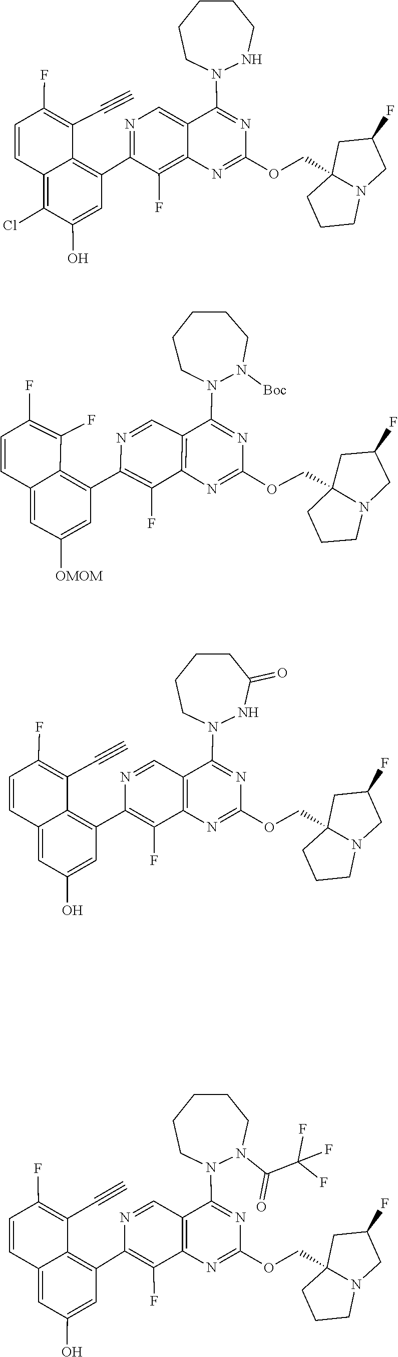

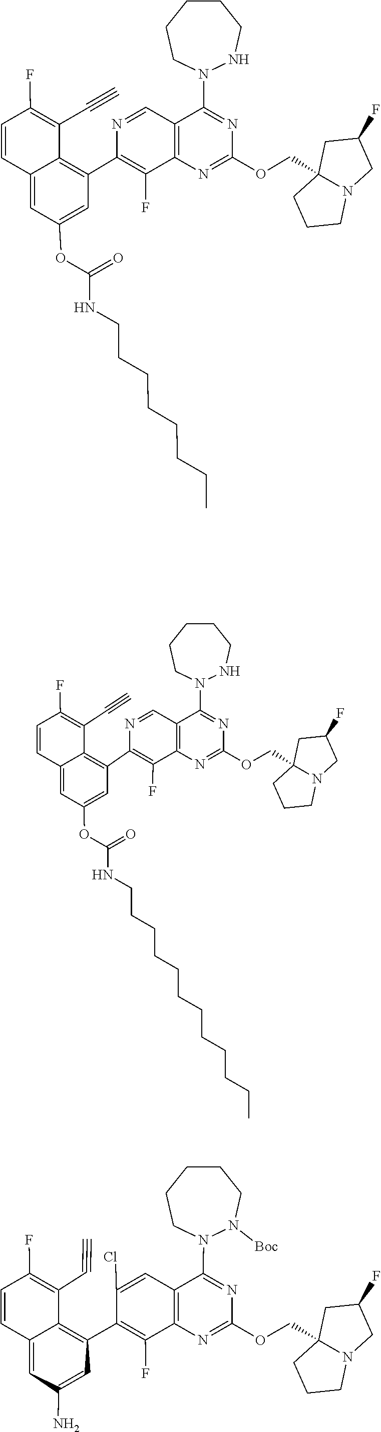

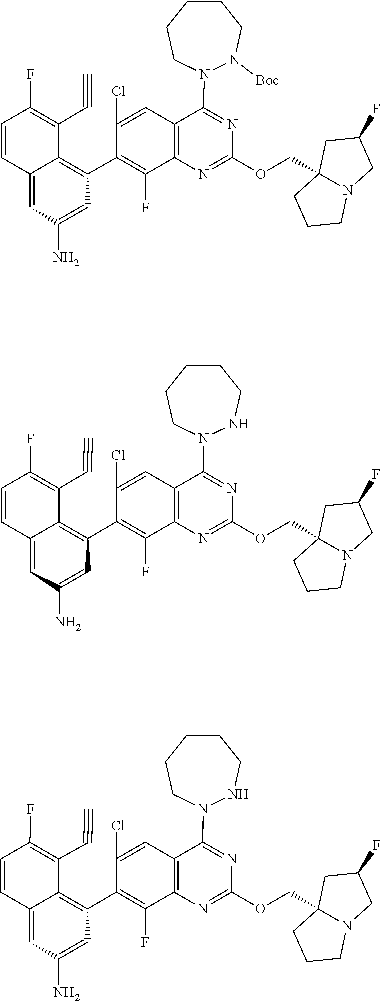

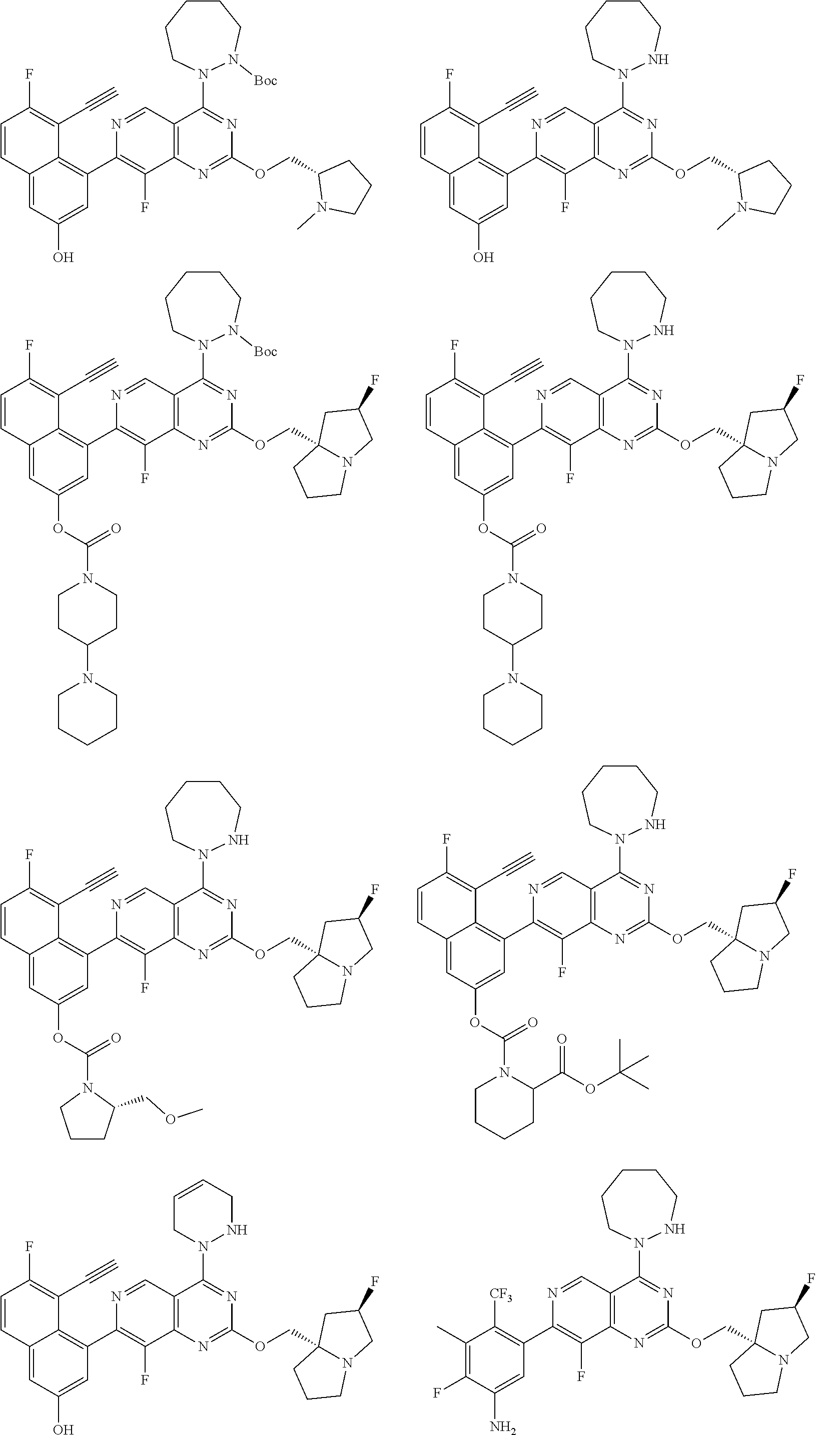

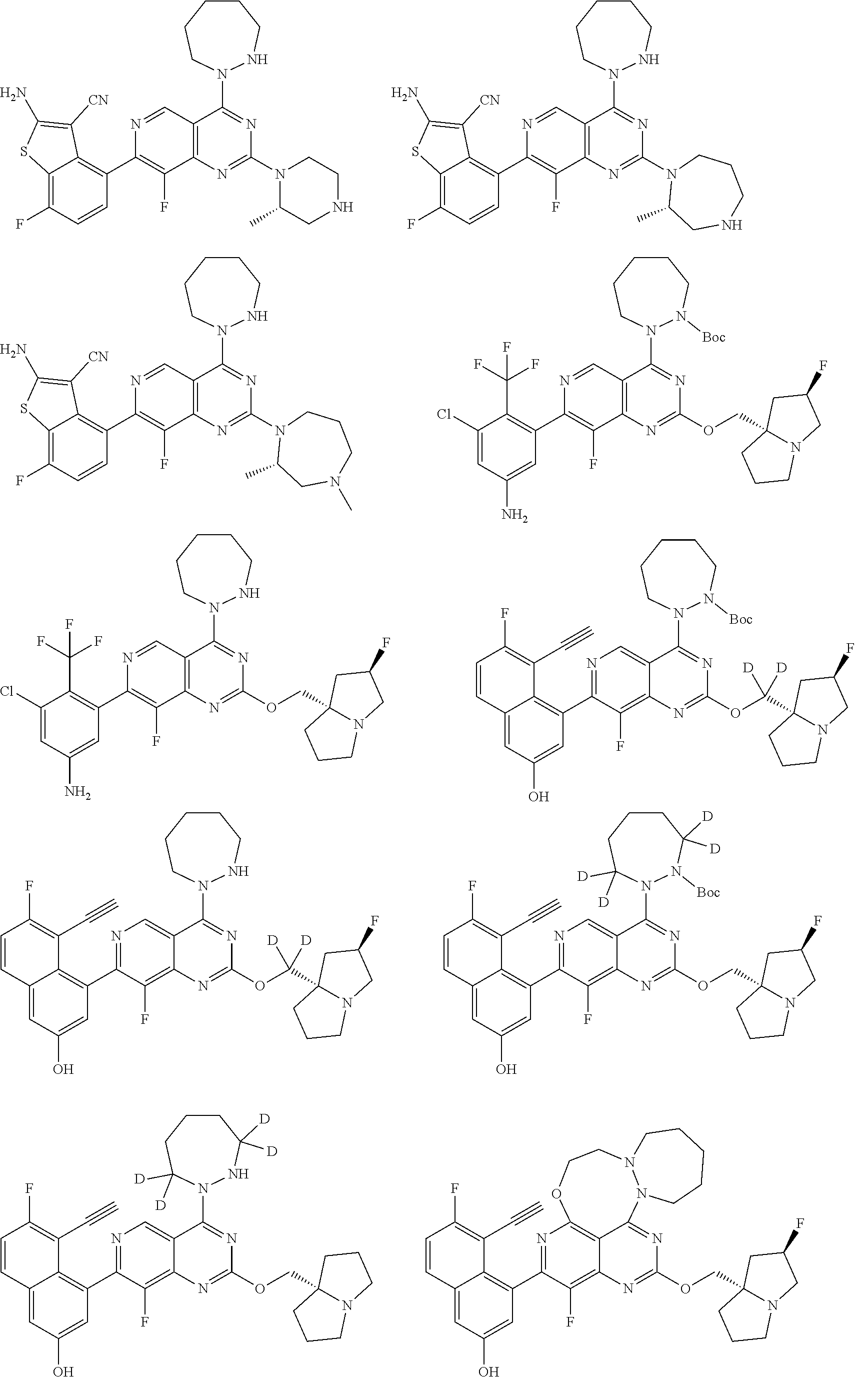

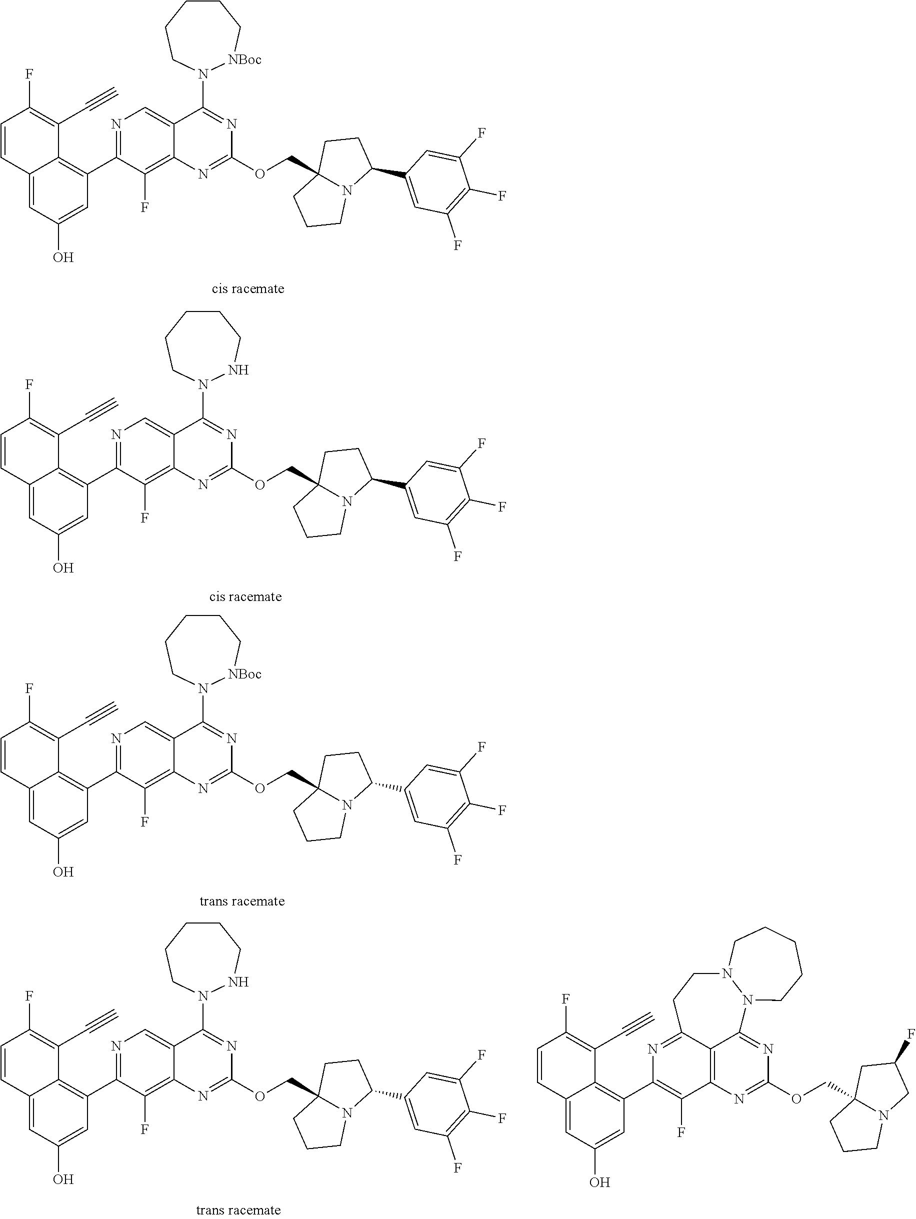

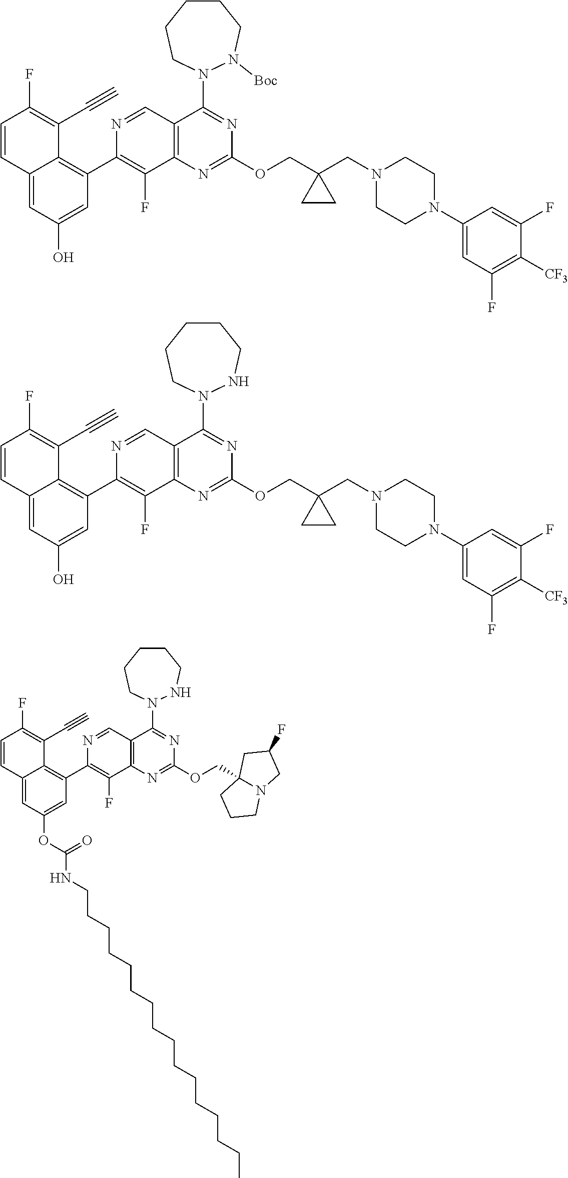

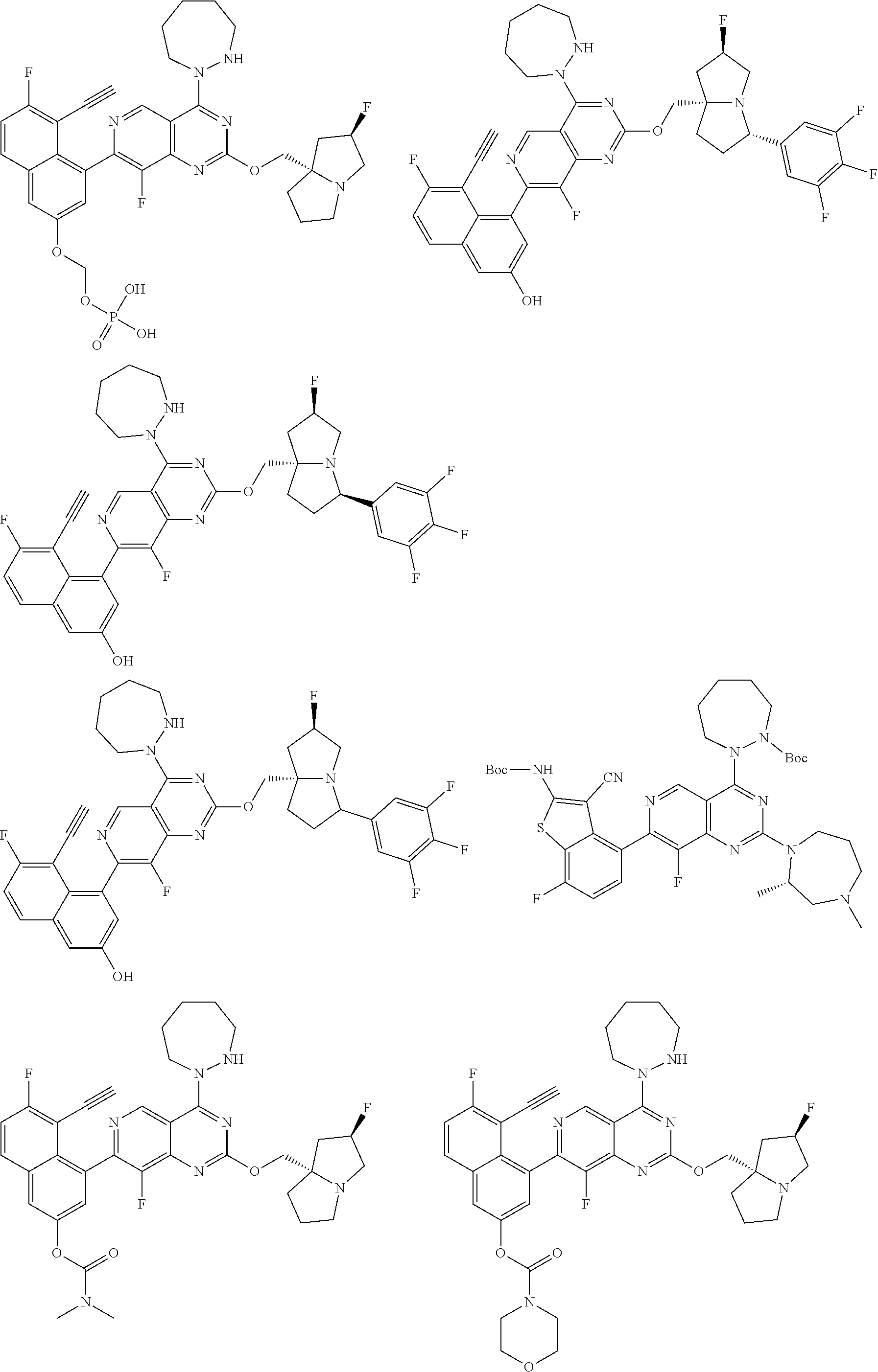

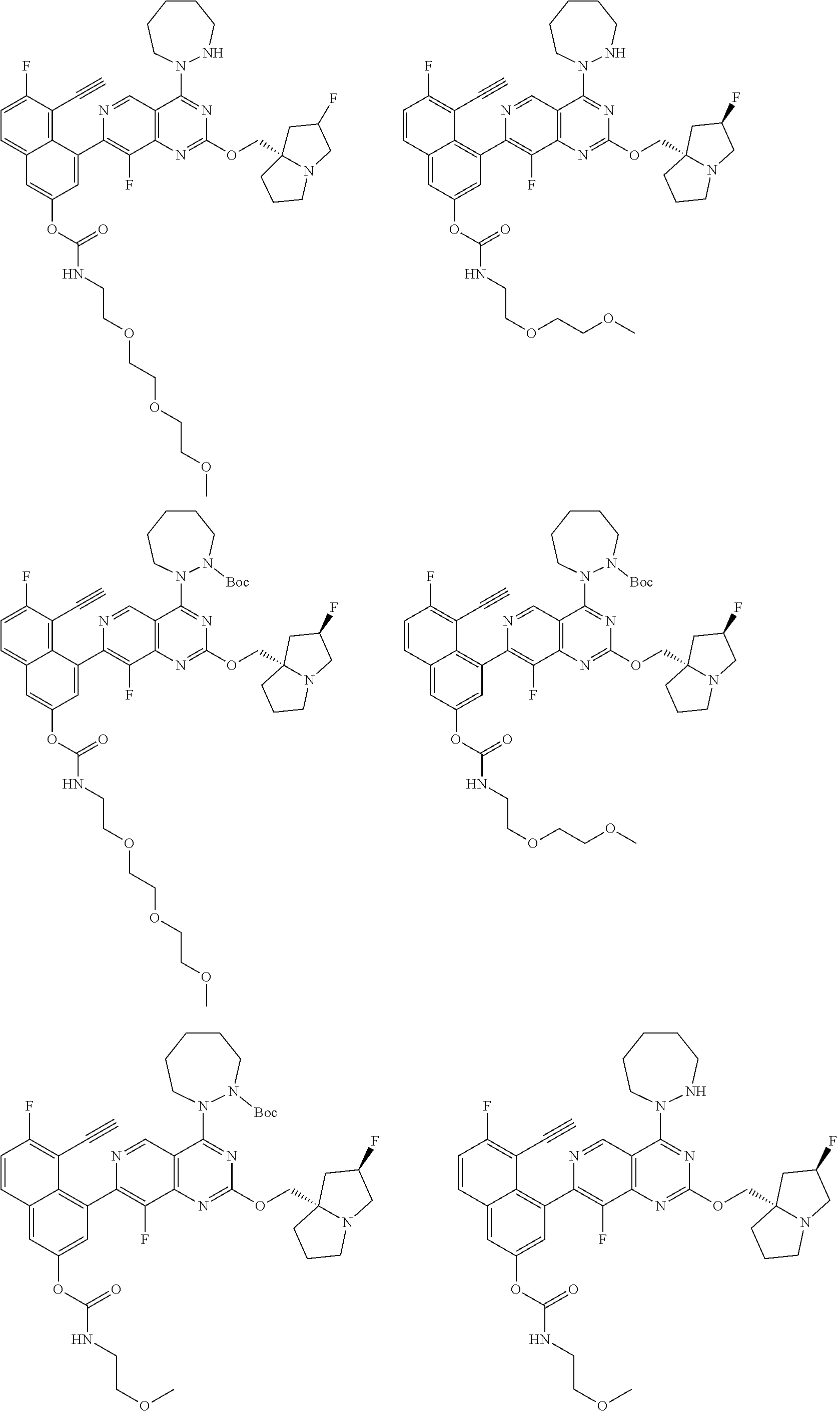

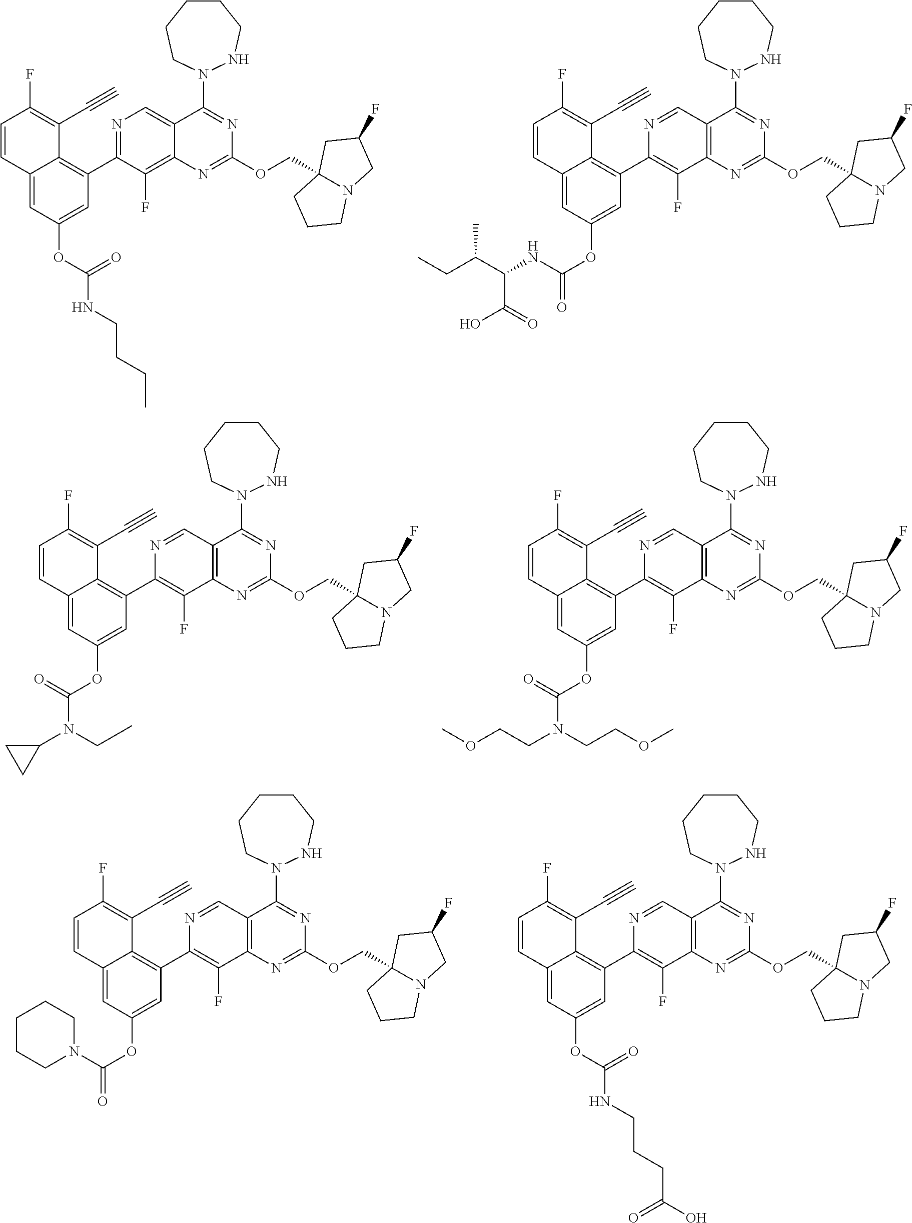

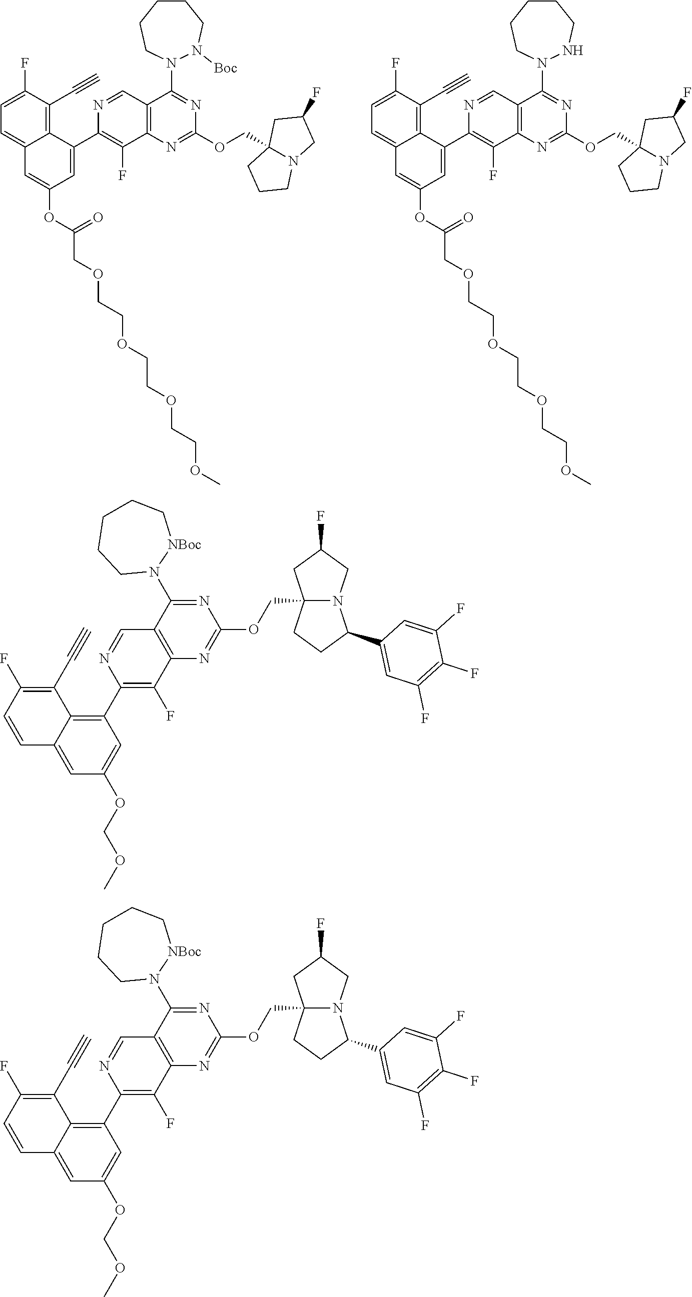

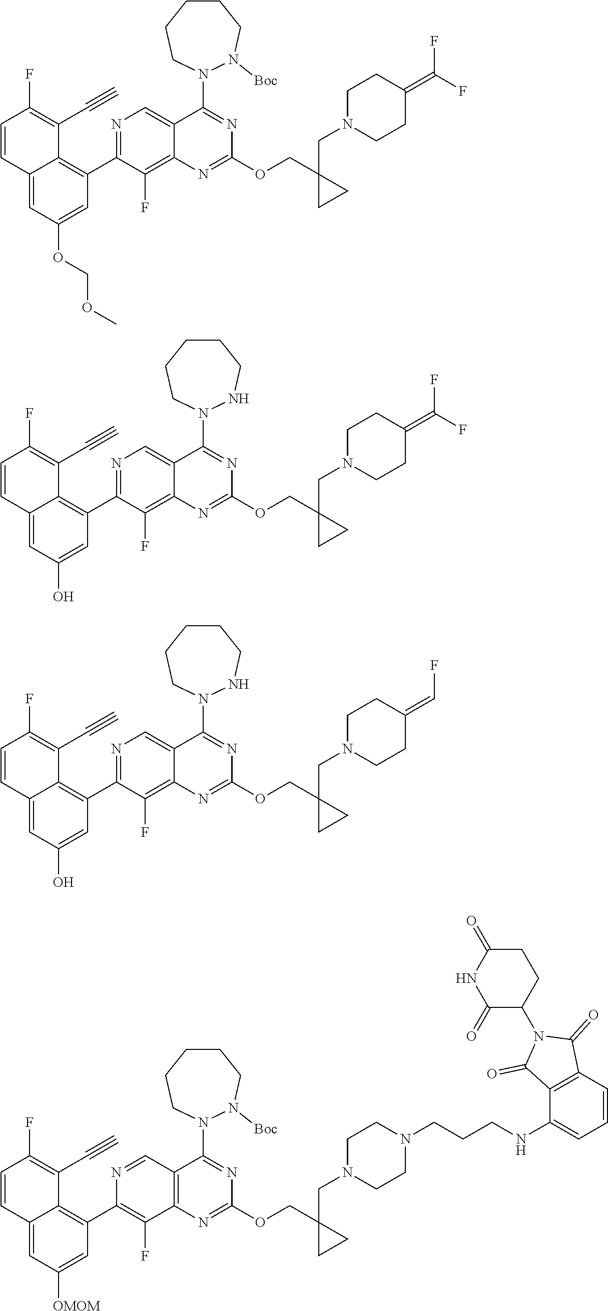

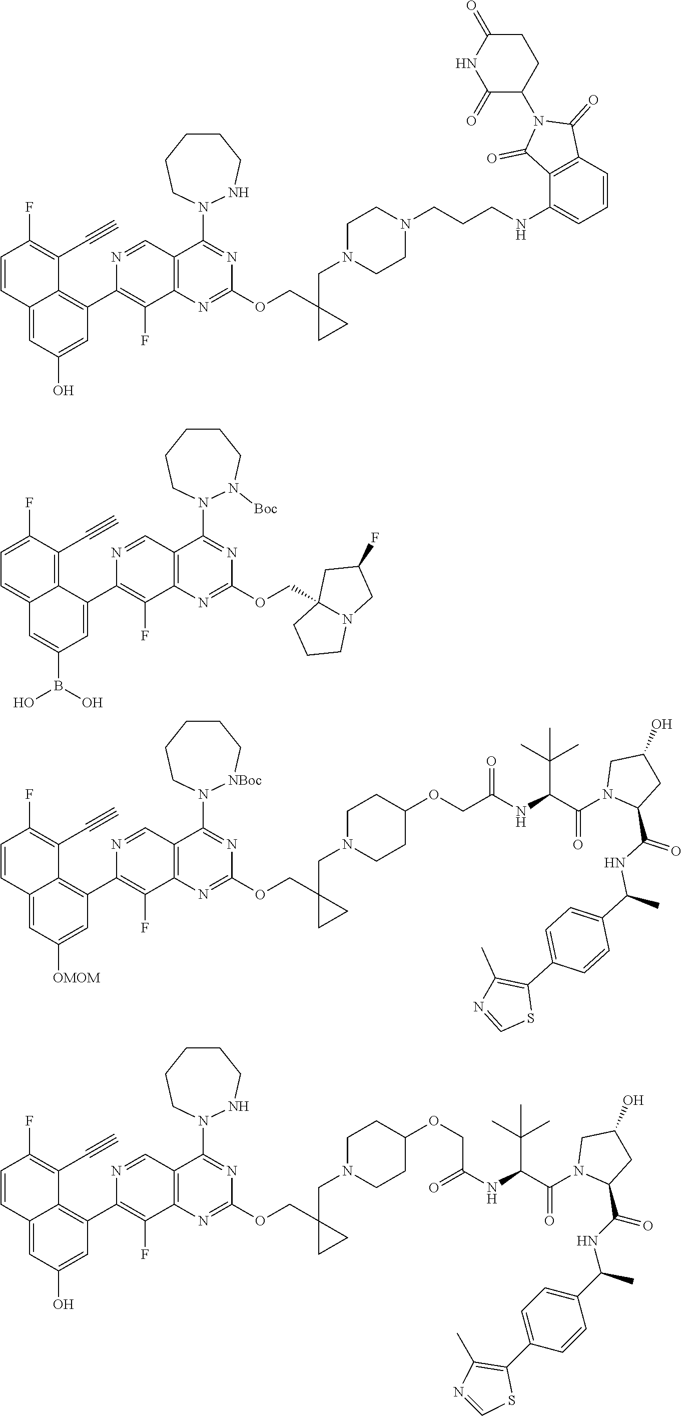

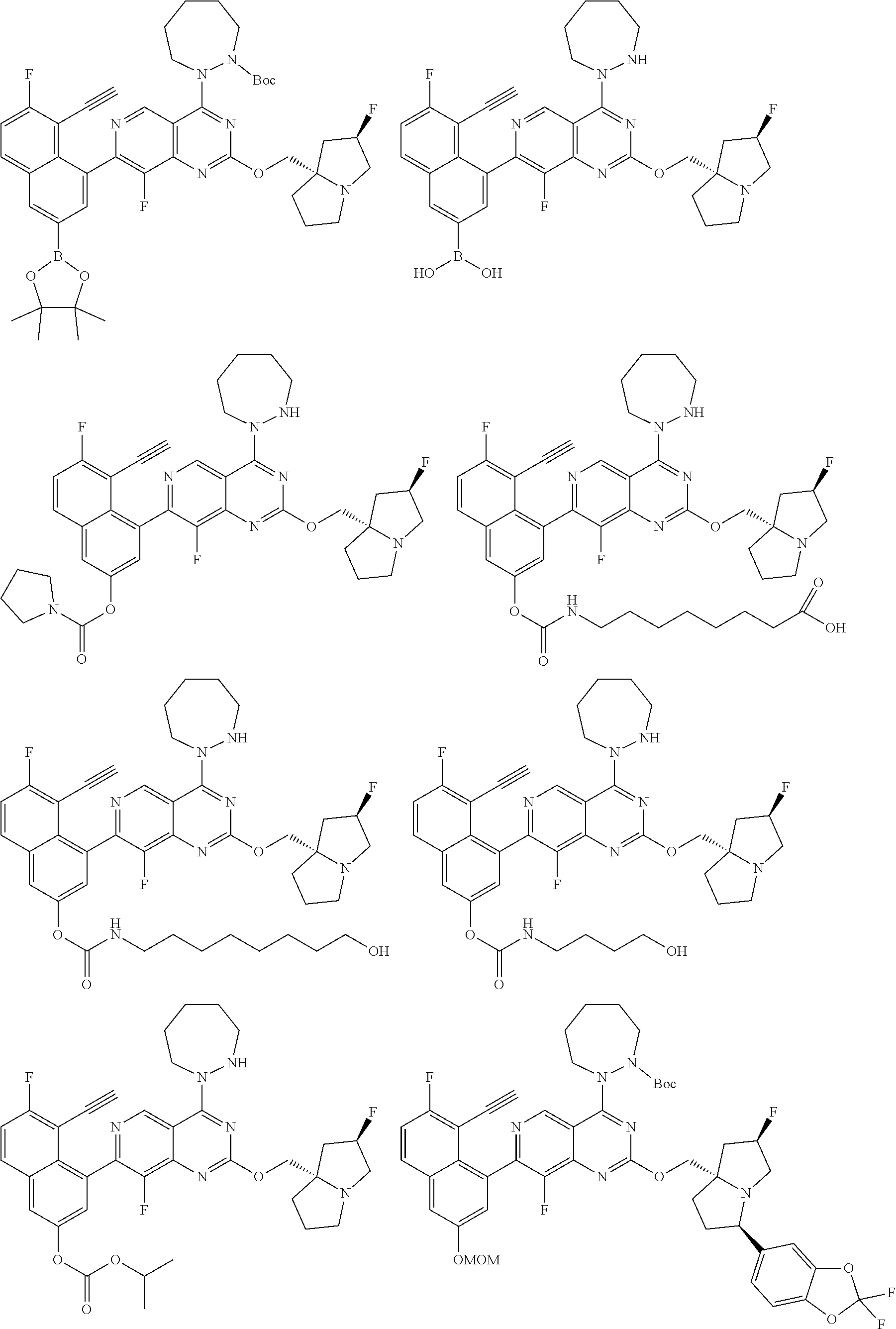

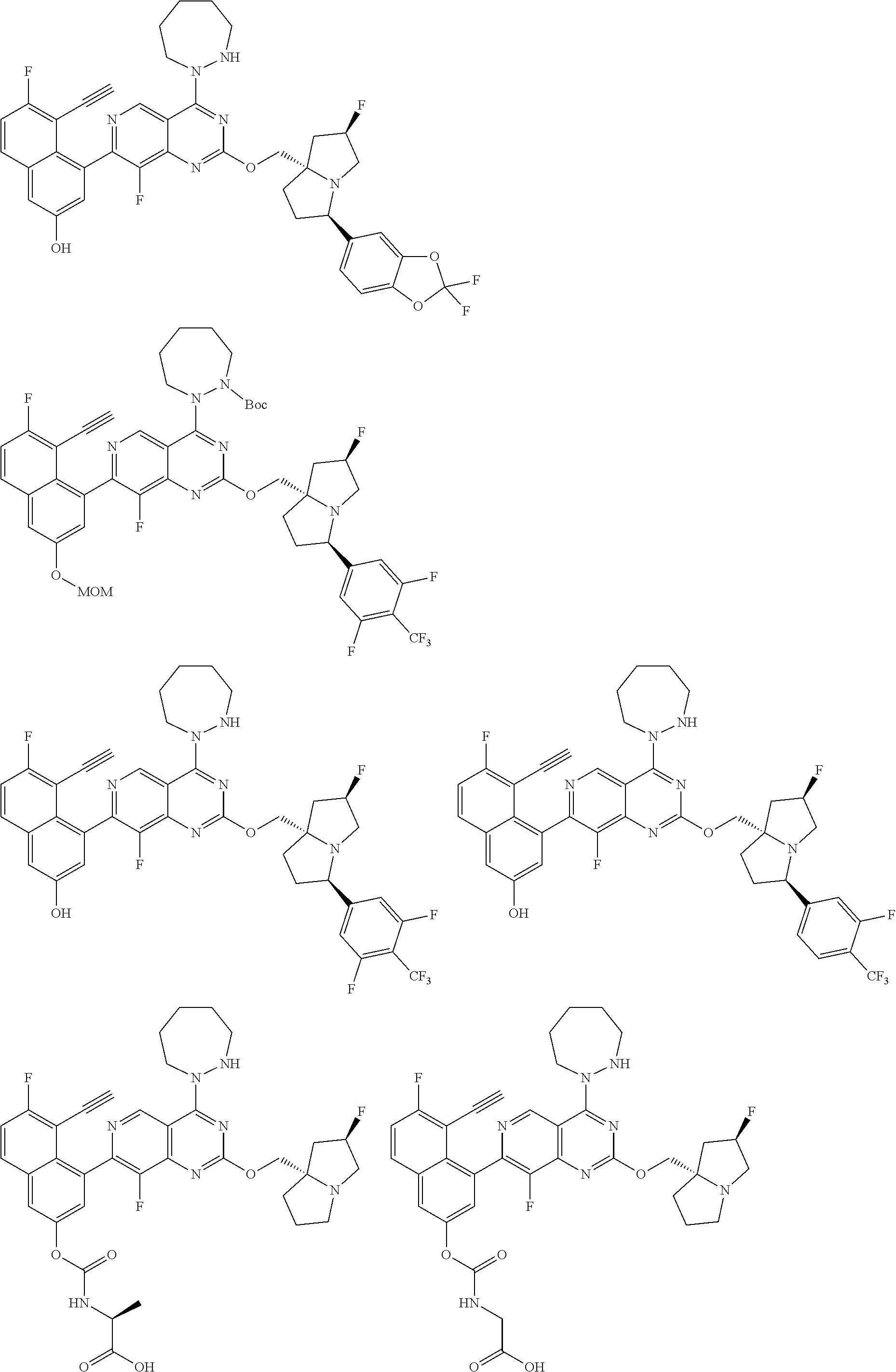

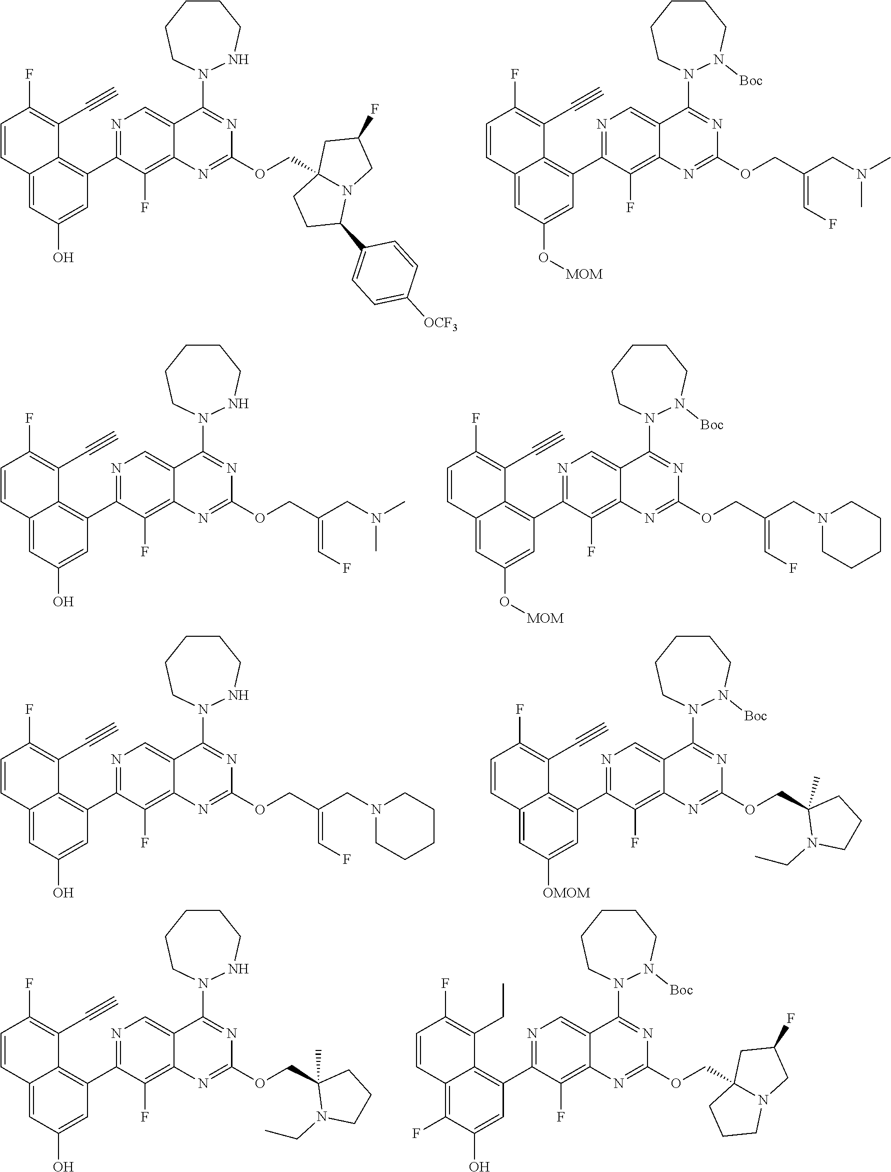

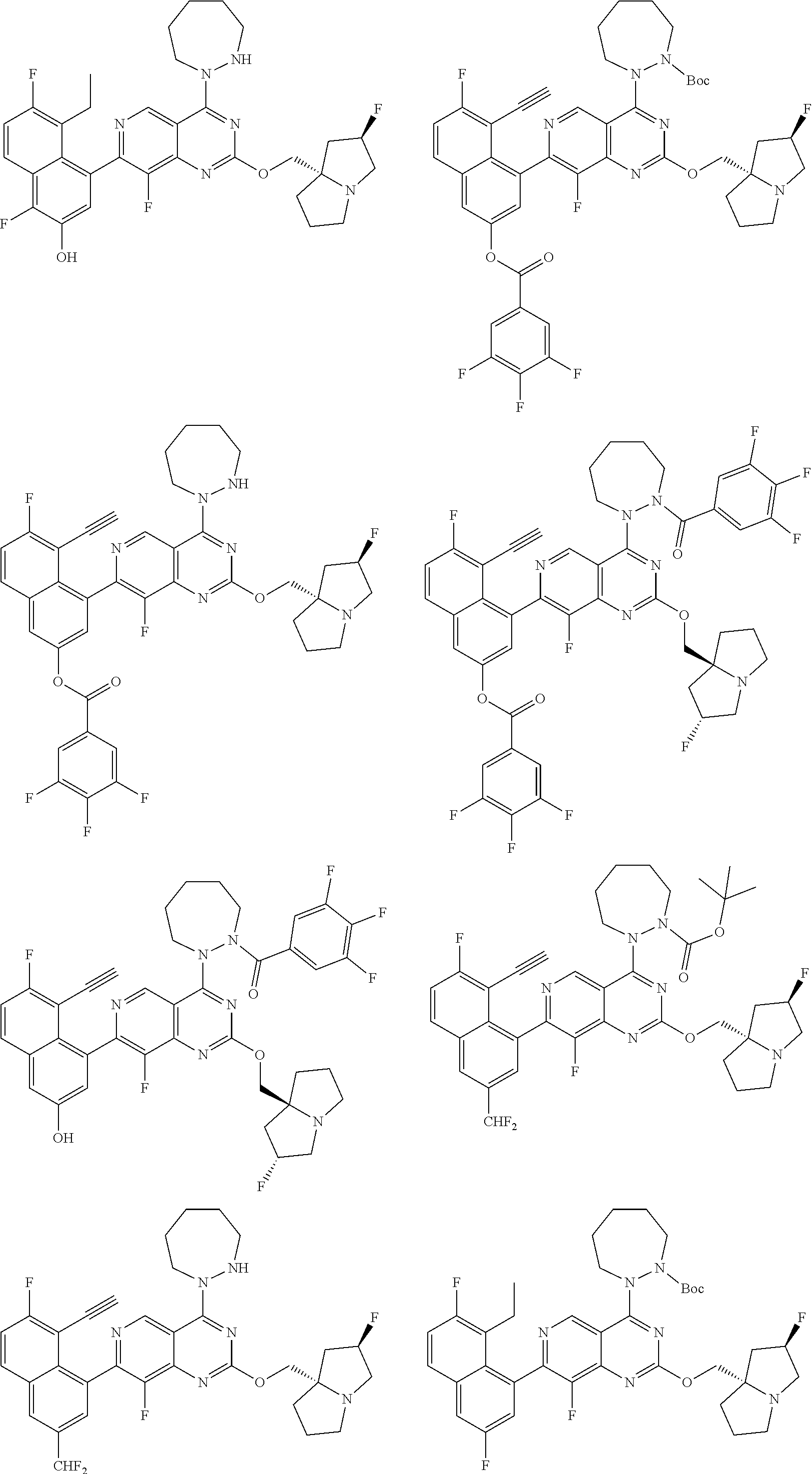

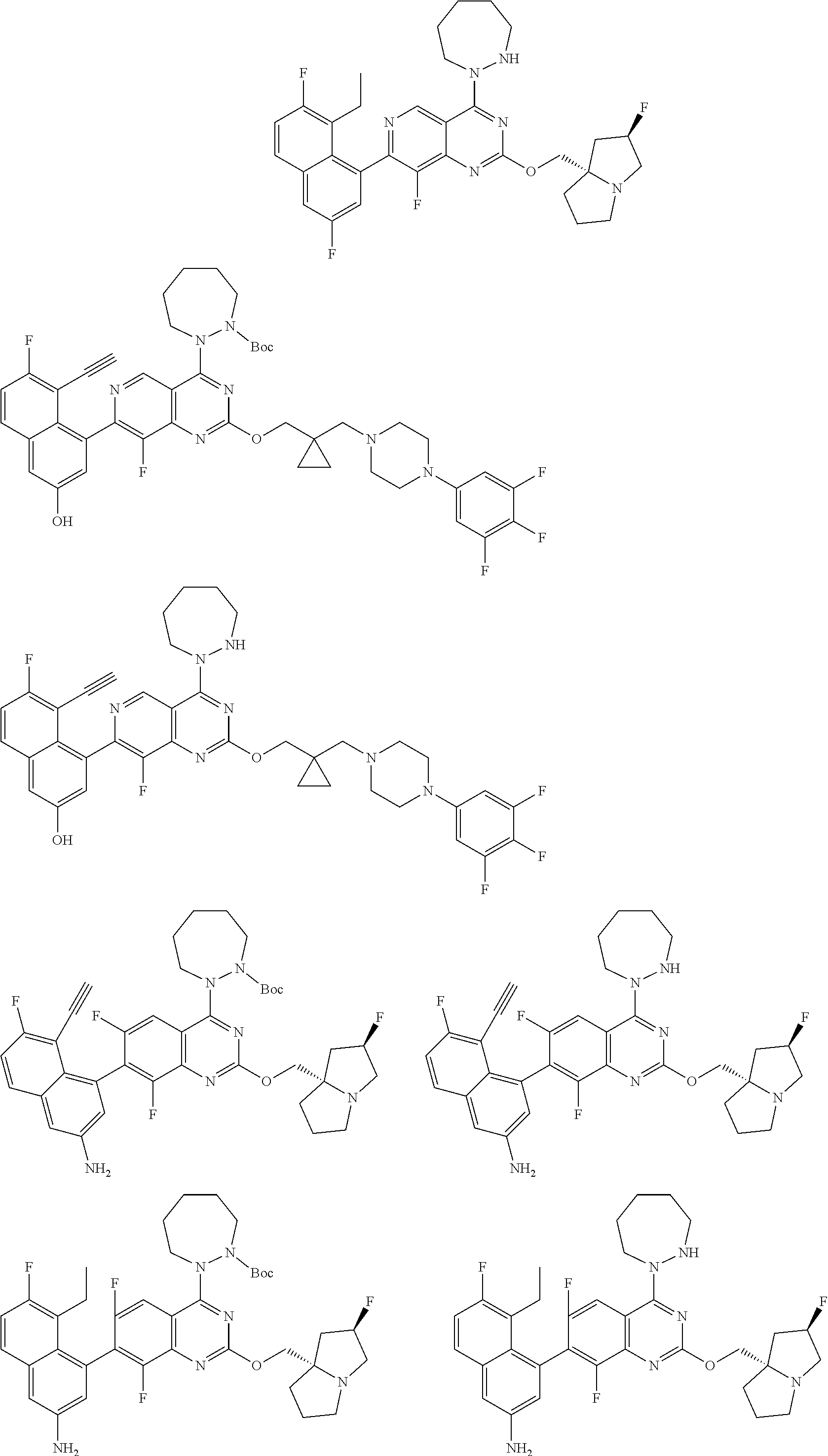

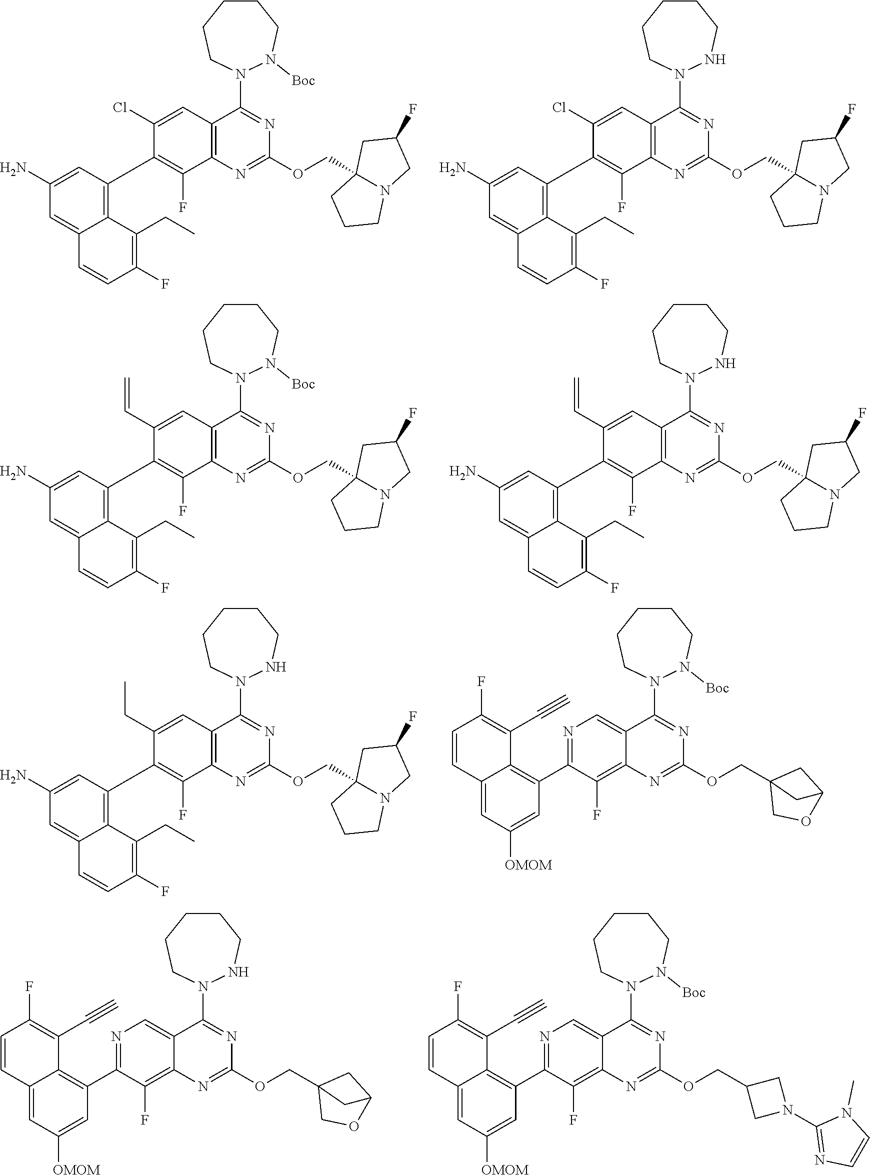

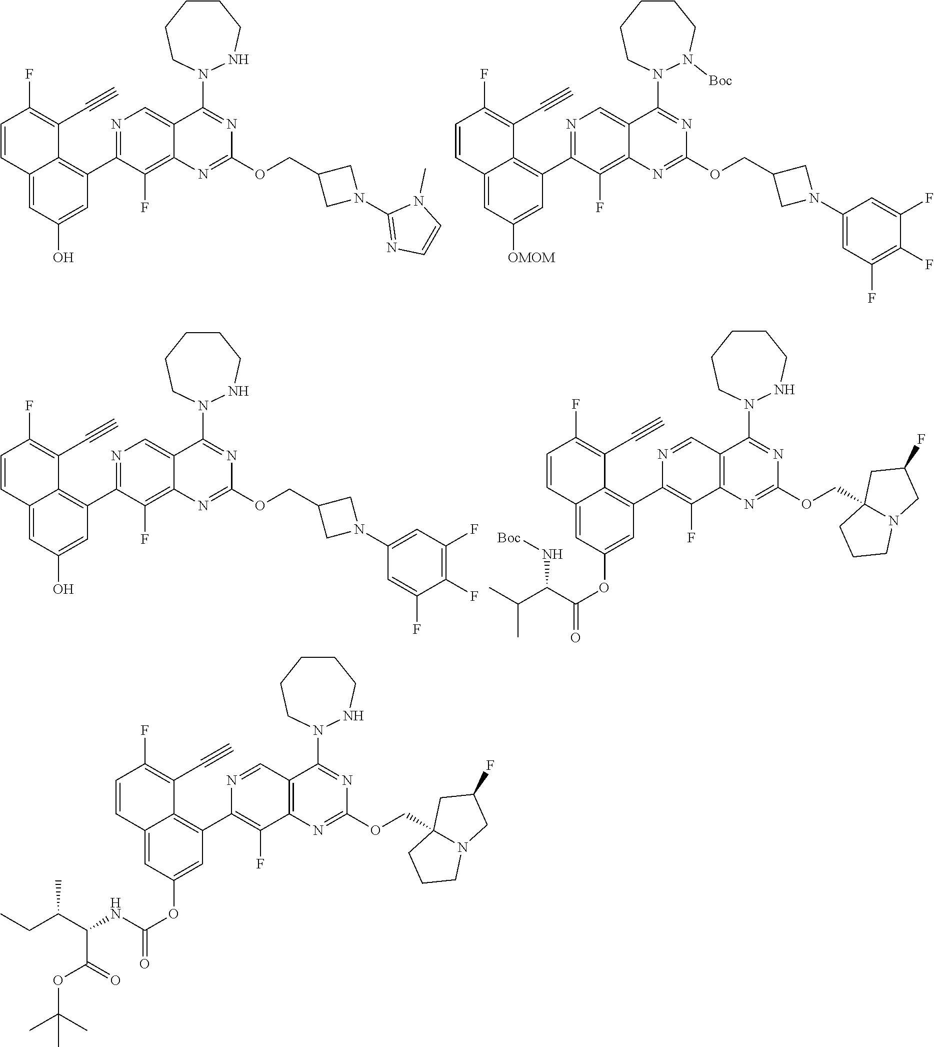

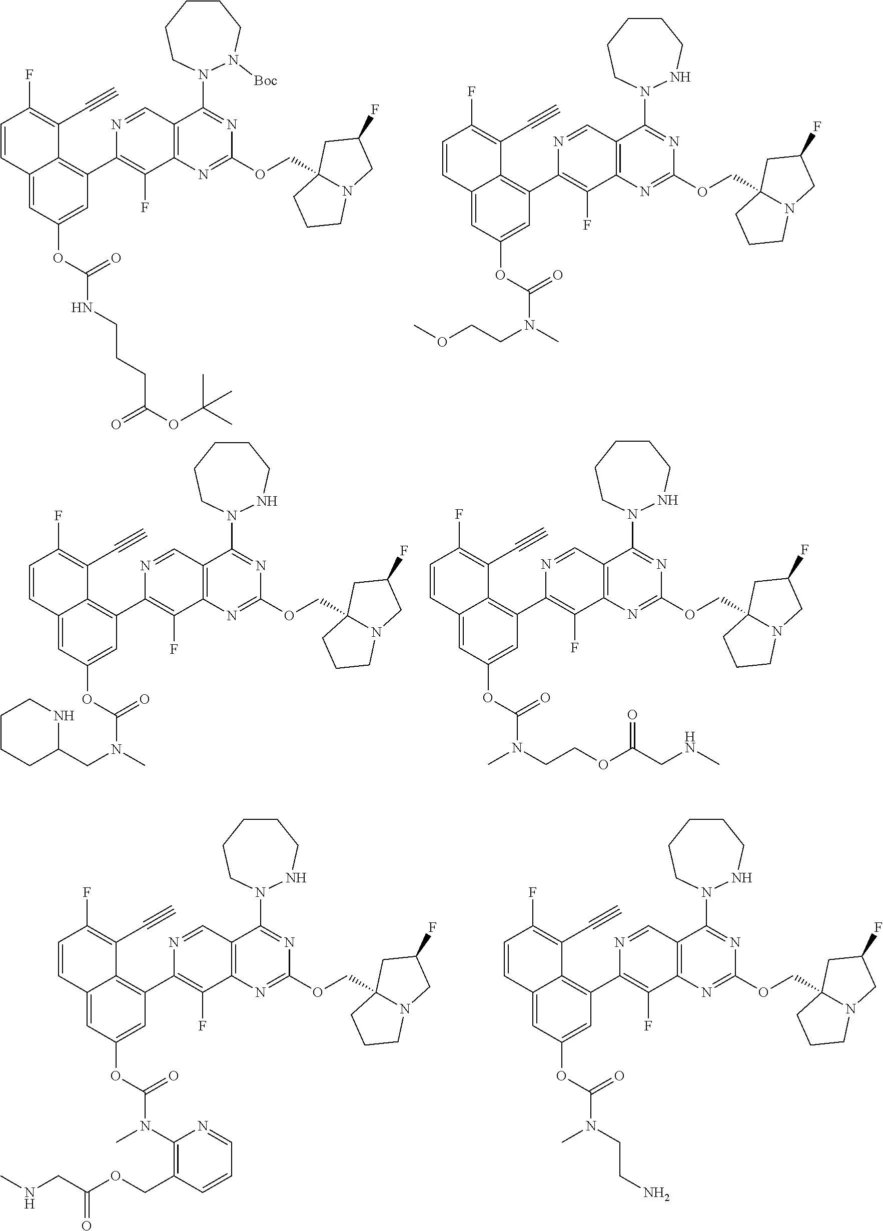

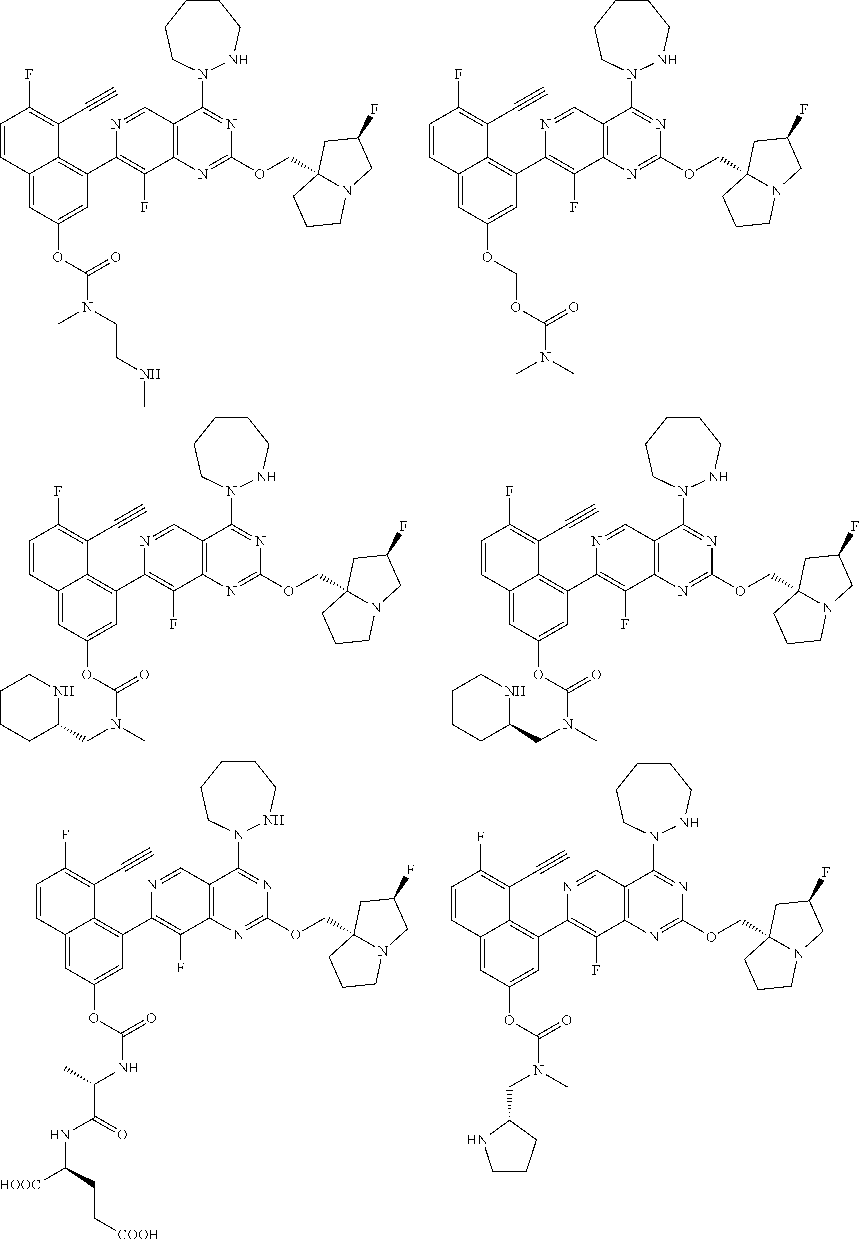

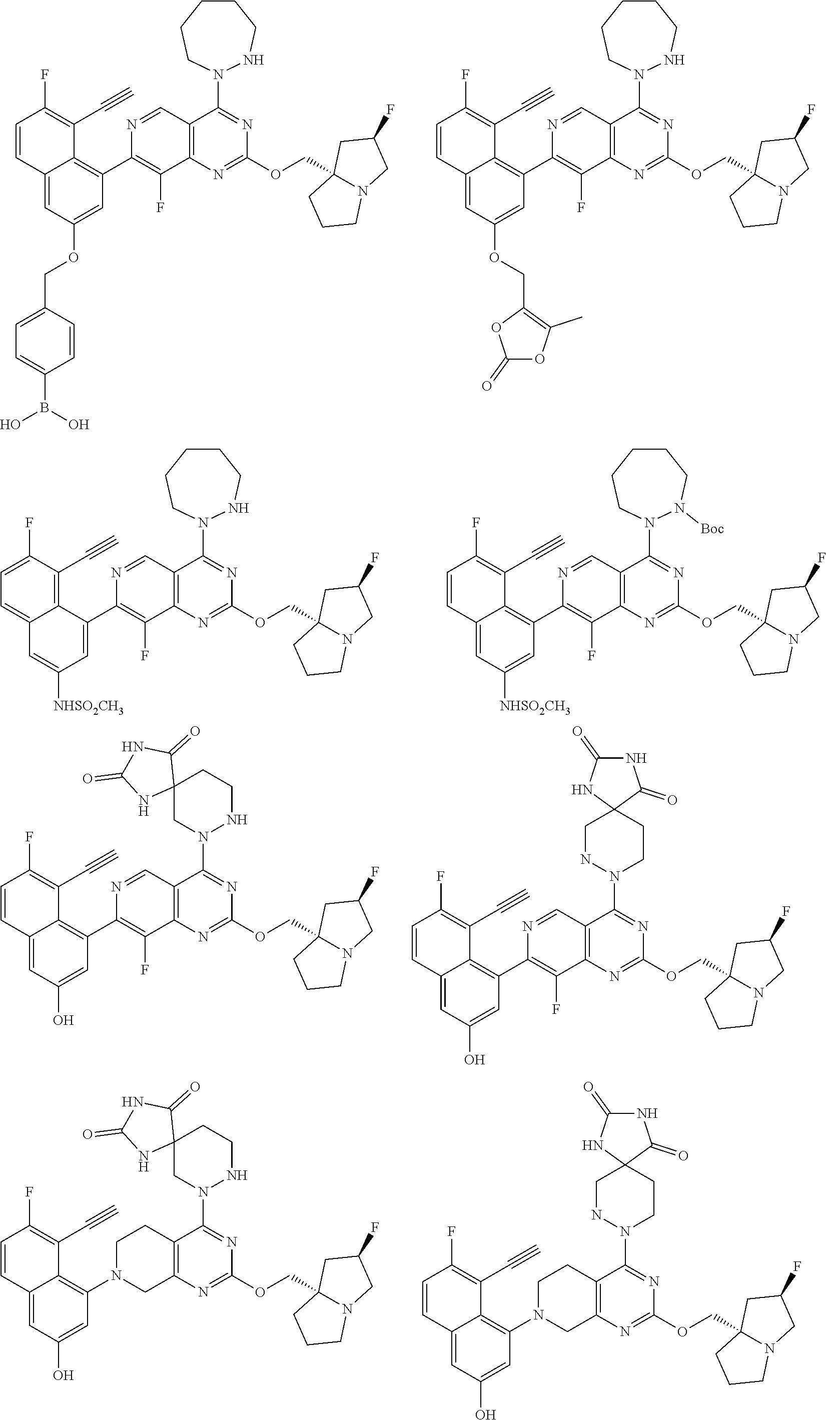

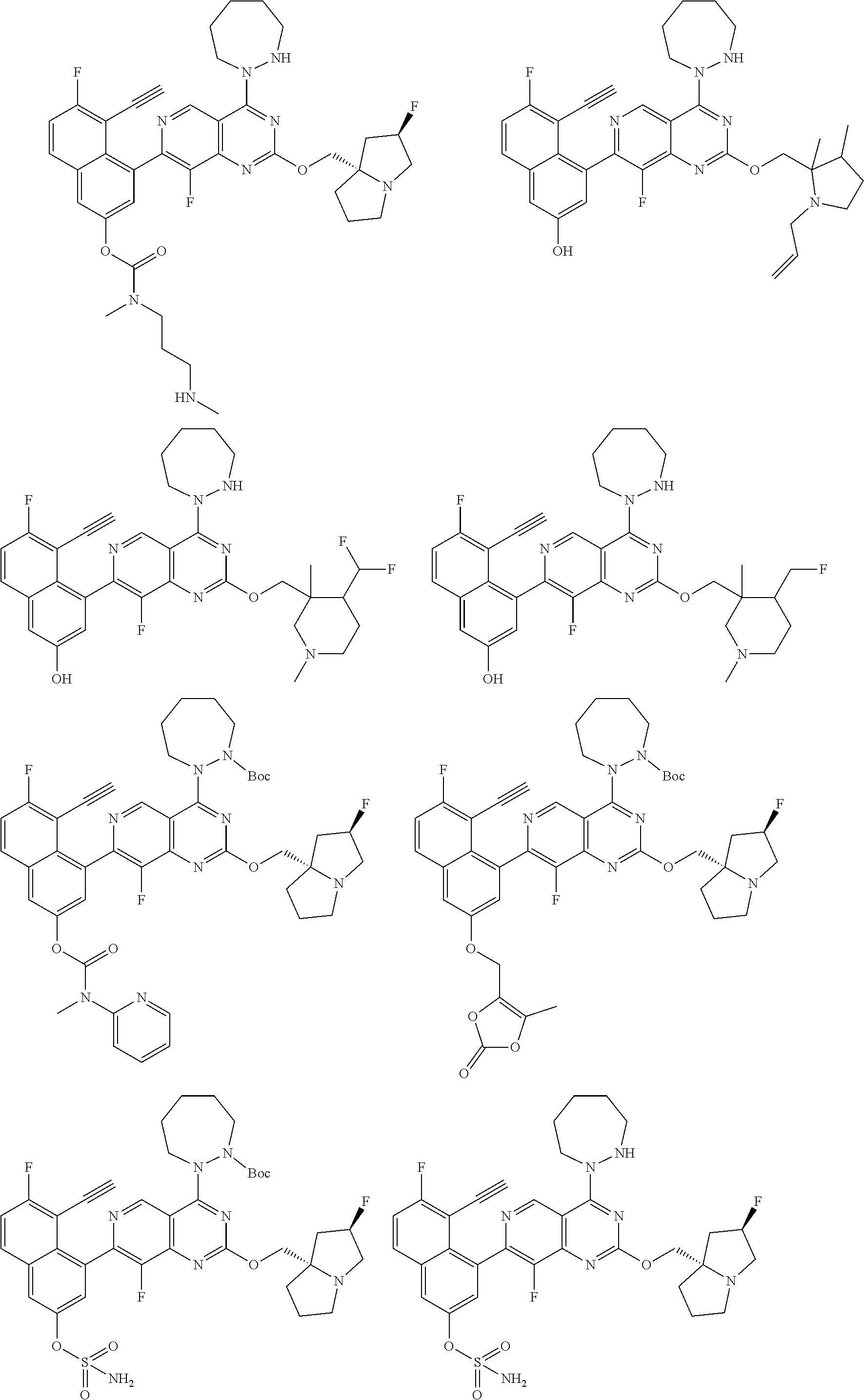

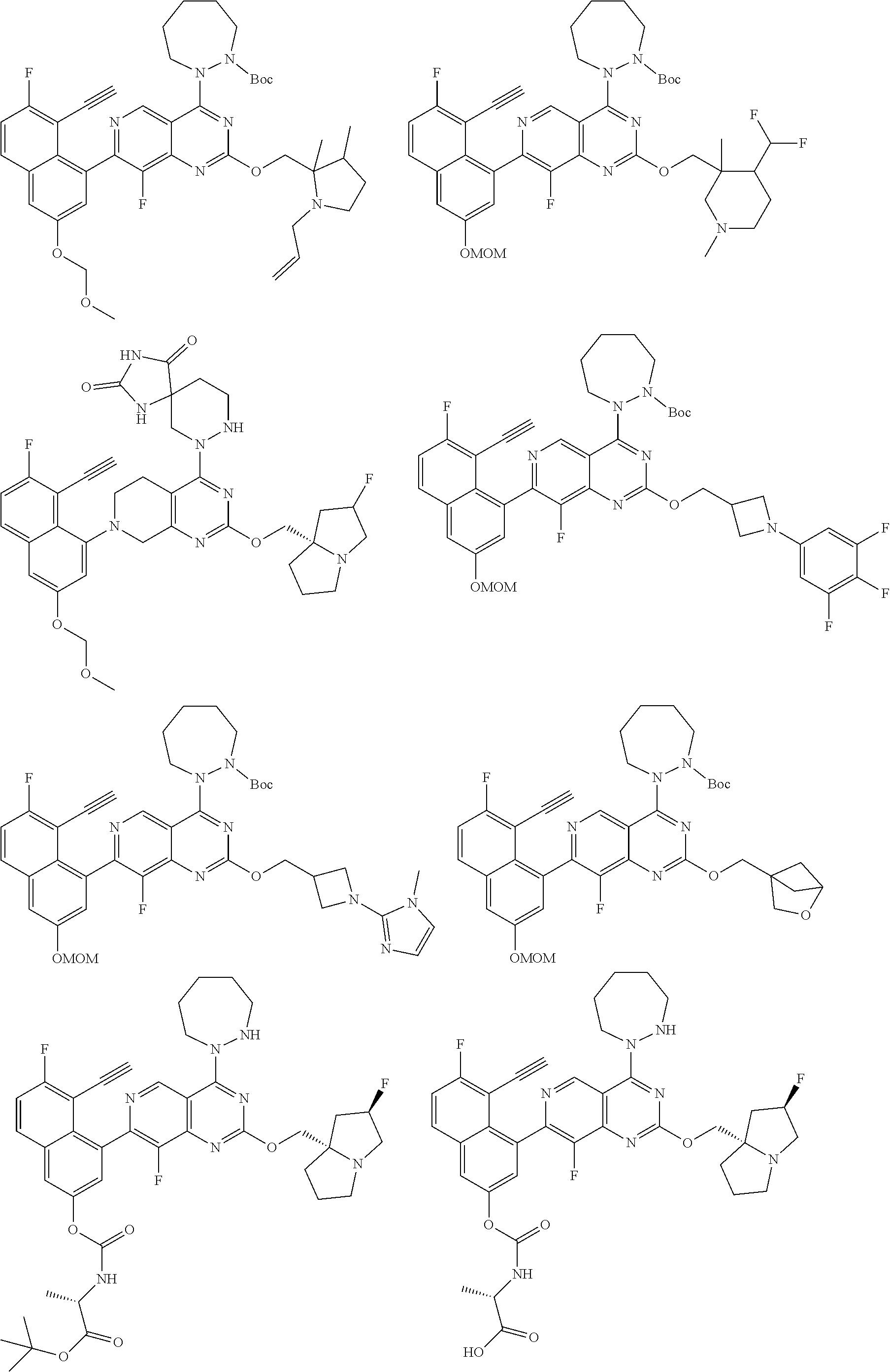

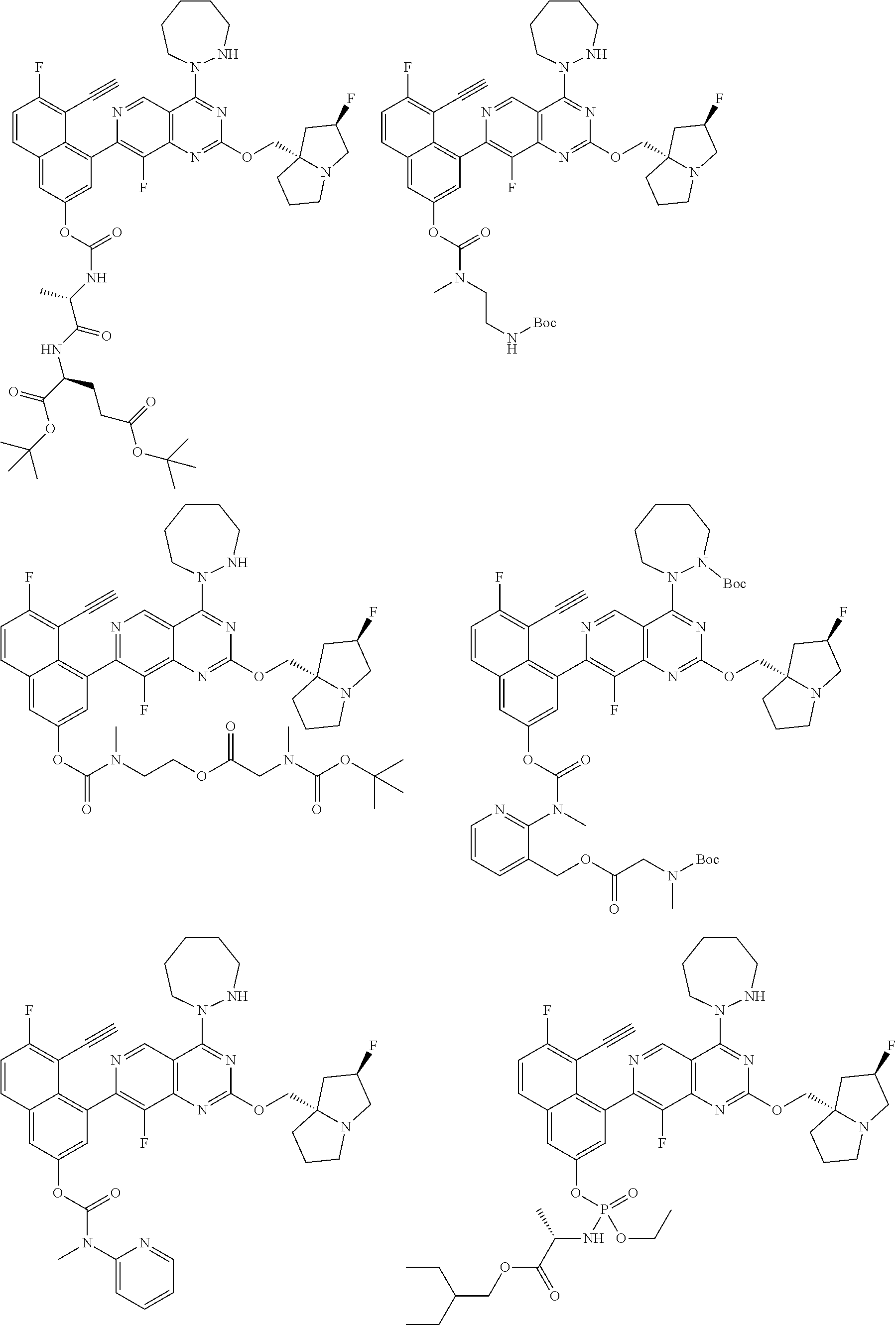

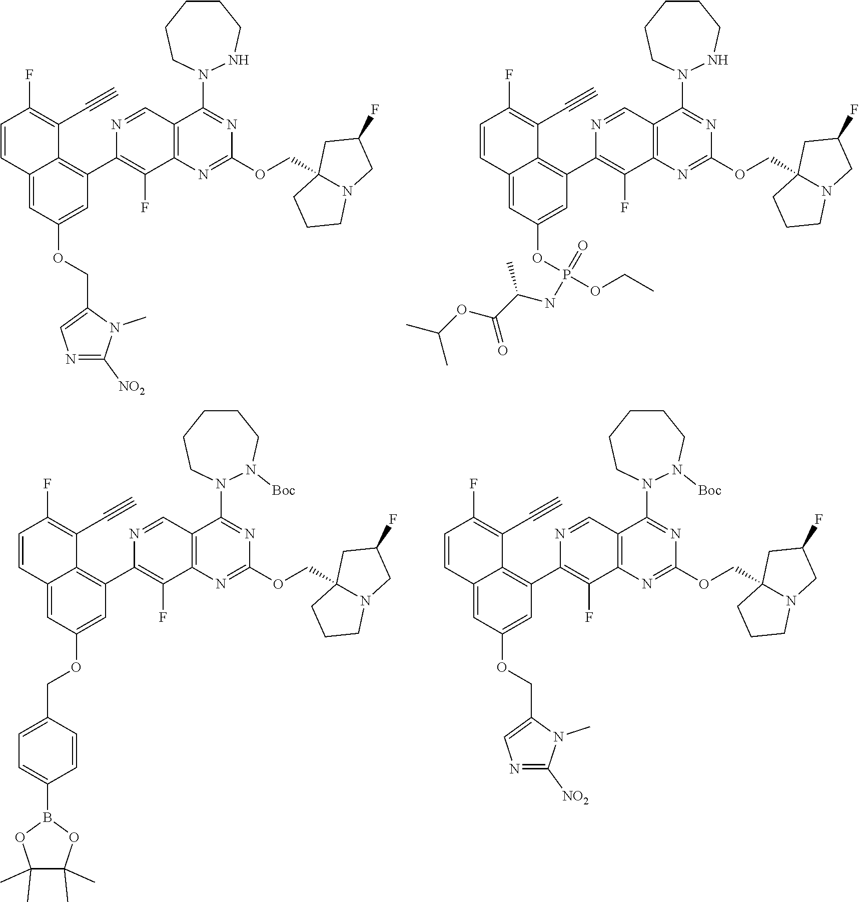

9. A compound has any one of the following structures:

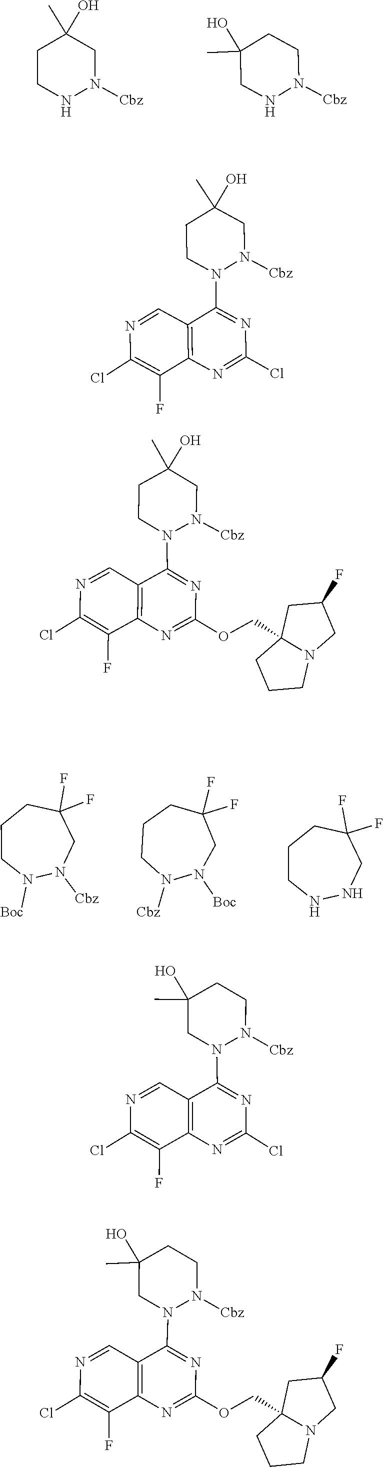

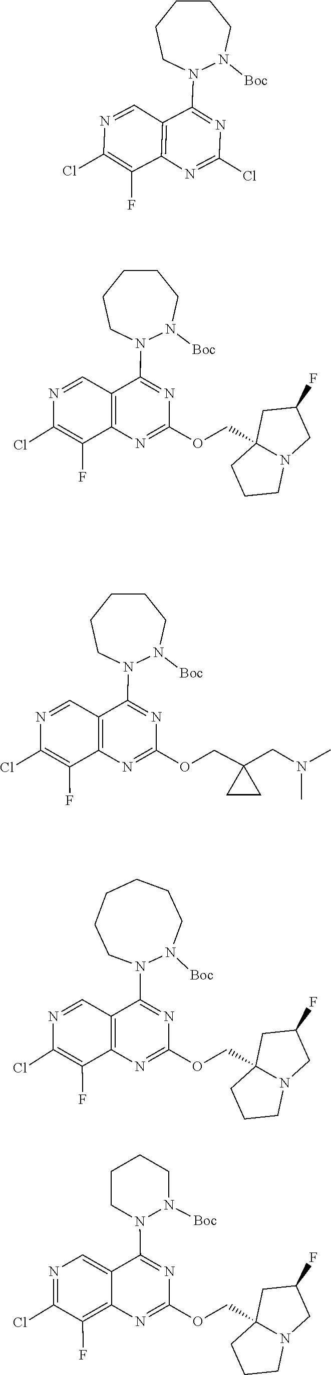 [SMILES: CC(C)(C)OC(=O)N1CCCCCN1c1nc(Cl)nc2c(F)c(Cl)ncc12 ; CC(C)(C)OC(=O)N1CCCCCN1c1nc(OC[C@@]23CCCN2C[C@H](F)C3)nc2c(F)c(Cl)ncc12 ; CN(C)CC1(COc2nc(N3CCCCCN3C(=O)OC(C)(C)C)c3cnc(Cl)c(F)c3n2)CC1 ; CC(C)(C)OC(=O)N1CCCCCCN1c1nc(OC[C@@]23CCCN2C[C@H](F)C3)nc2c(F)c(Cl)ncc12 ; CC(C)(C)OC(=O)N1CCCCN1c1nc(OC[C@@]23CCCN2C[C@H](F)C3)nc2c(F)c(Cl)ncc12]

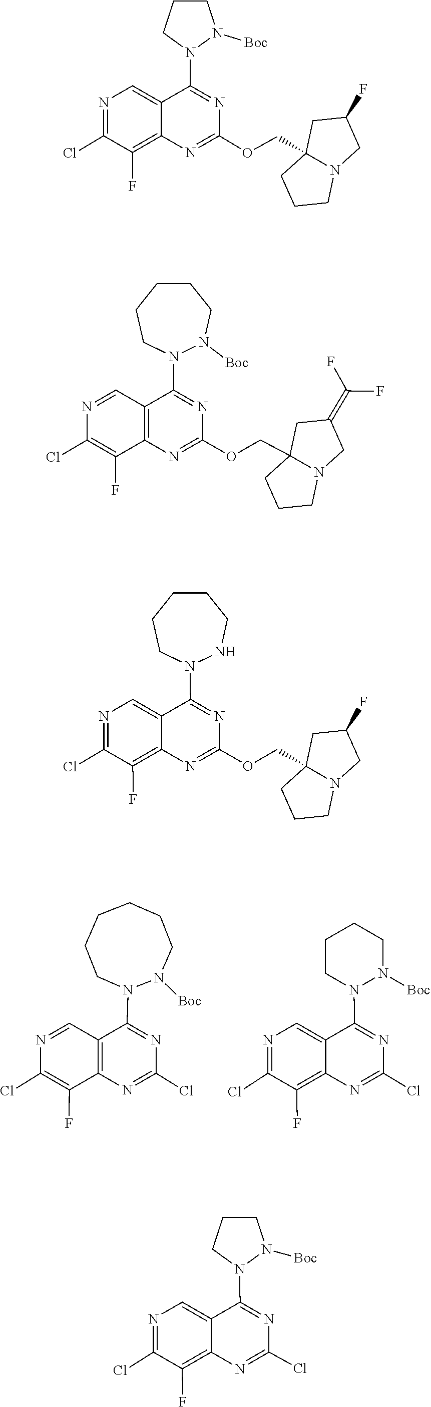 [SMILES: CC(C)(C)OC(=O)N1CCCN1c1nc(OC[C@@]23CCCN2C[C@H](F)C3)nc2c(F)c(Cl)ncc12 ; CC(C)(C)OC(=O)N1CCCCCN1c1nc(OCC23CCCN2CC(=C(F)F)C3)nc2c(F)c(Cl)ncc12 ; Fc1c(Cl)ncc2c(N3CCCCCN3)nc(OC[C@@]34CCCN3C[C@H](F)C4)nc12 ; CC(C)(C)OC(=O)N1CCCCCCN1c1nc(Cl)nc2c(F)c(Cl)ncc12 ; CC(C)(C)OC(=O)N1CCCCN1c1nc(Cl)nc2c(F)c(Cl)ncc12 ; CC(C)(C)OC(=O)N1CCCN1c1nc(Cl)nc2c(F)c(Cl)ncc12]

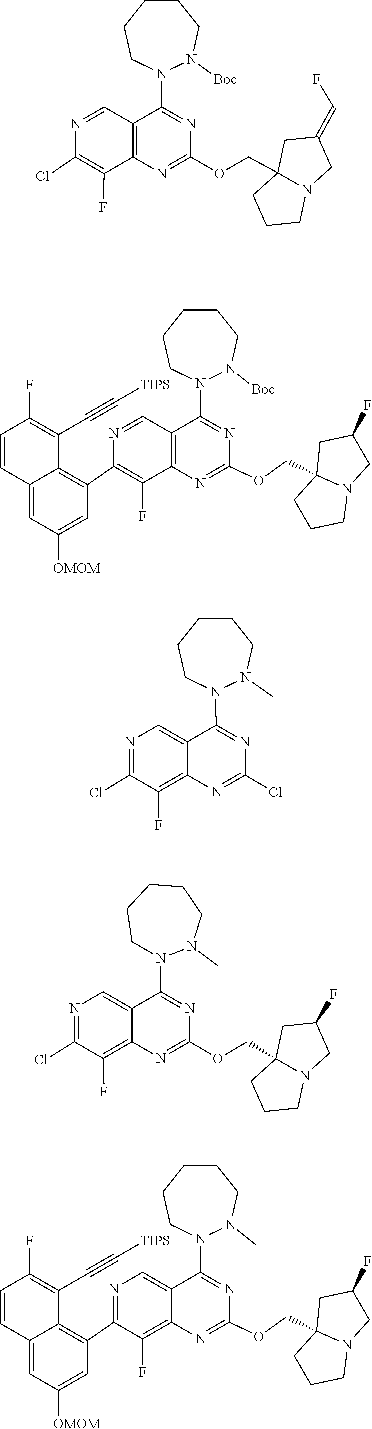

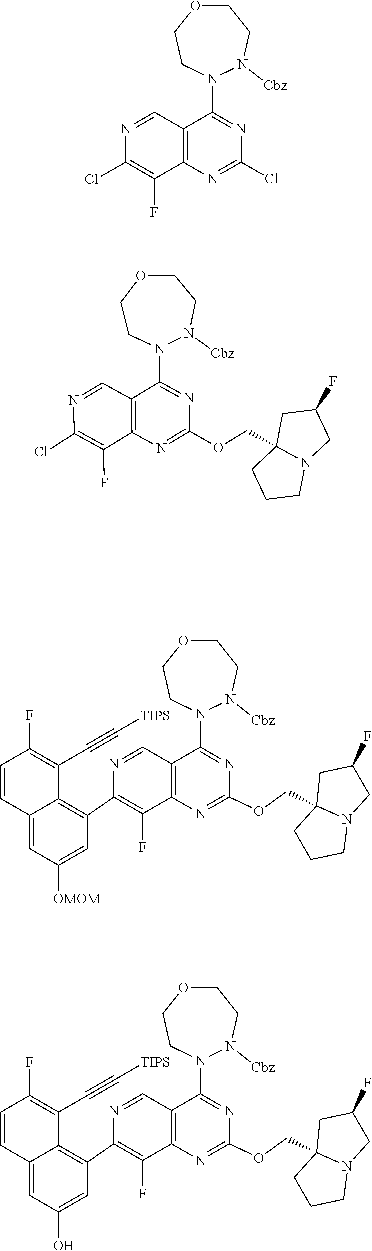

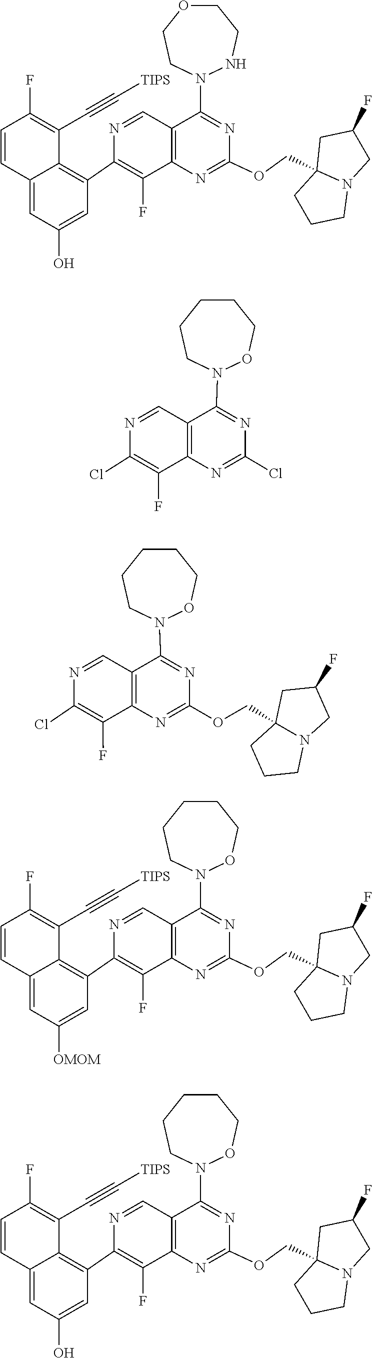

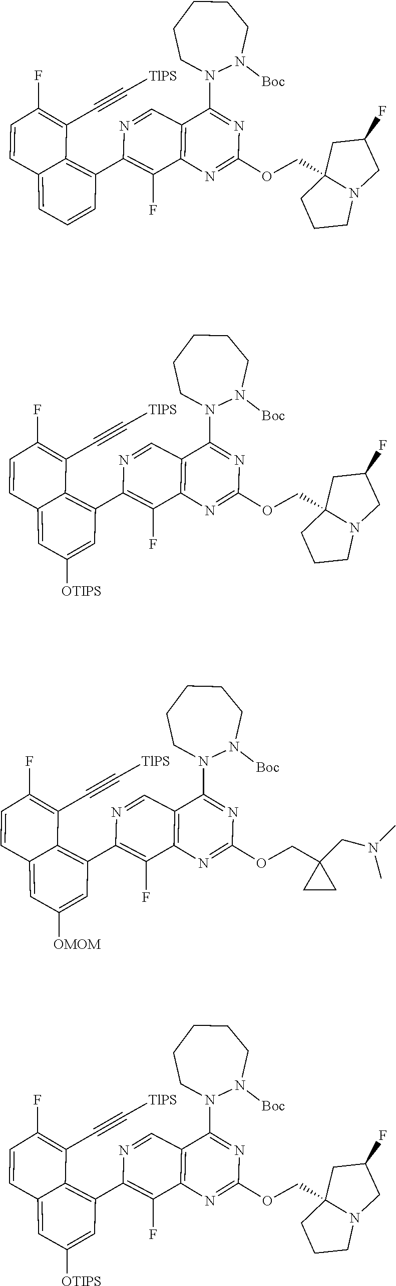

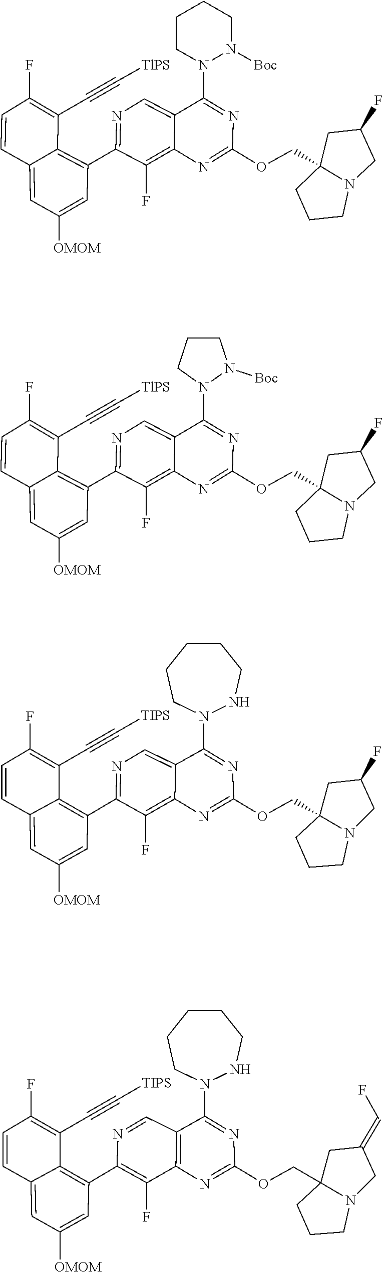

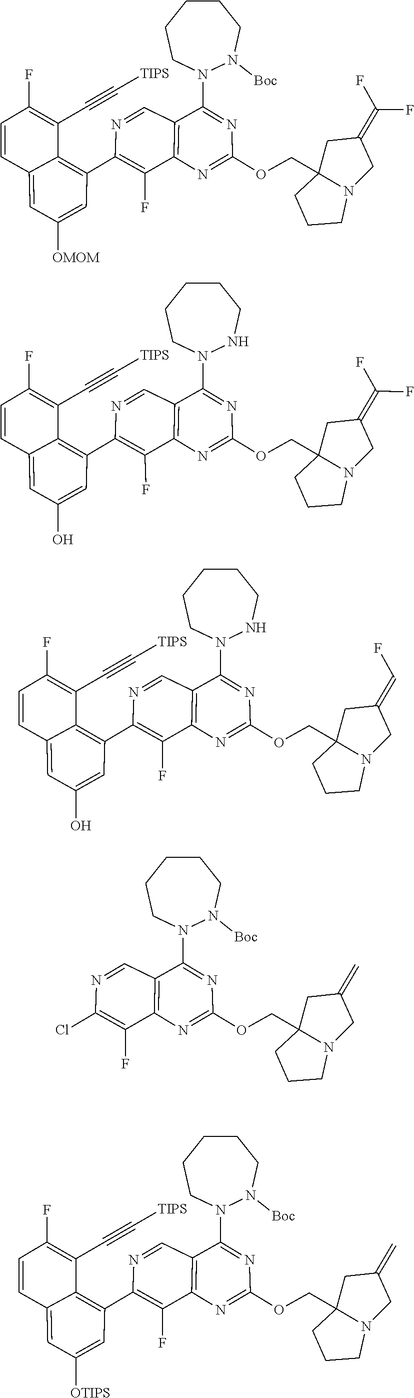

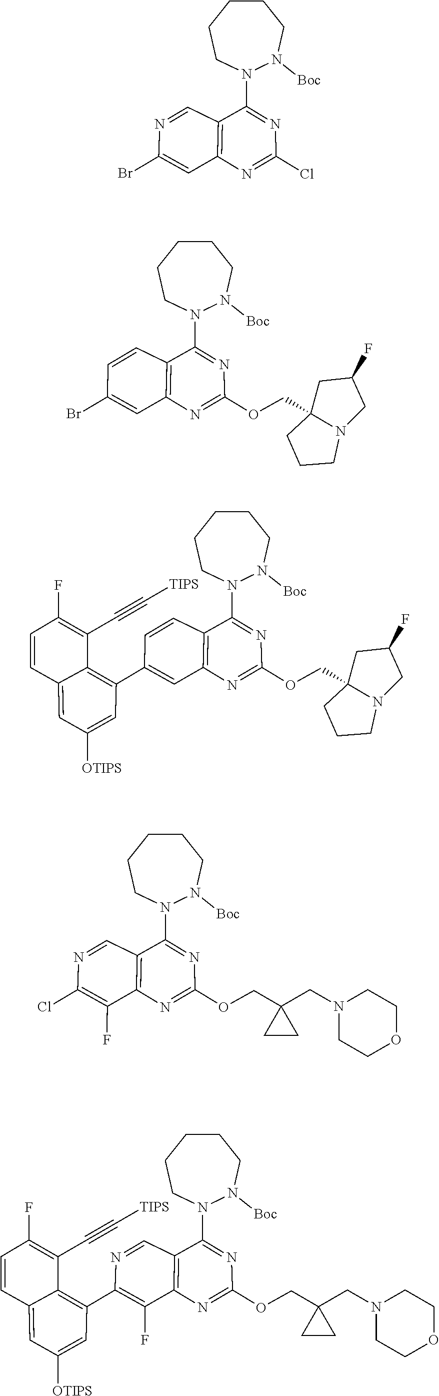

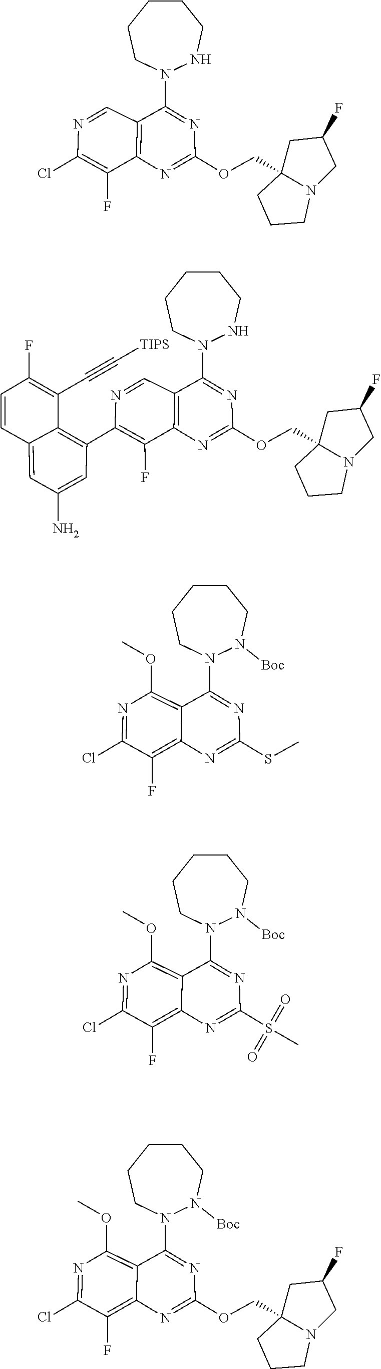

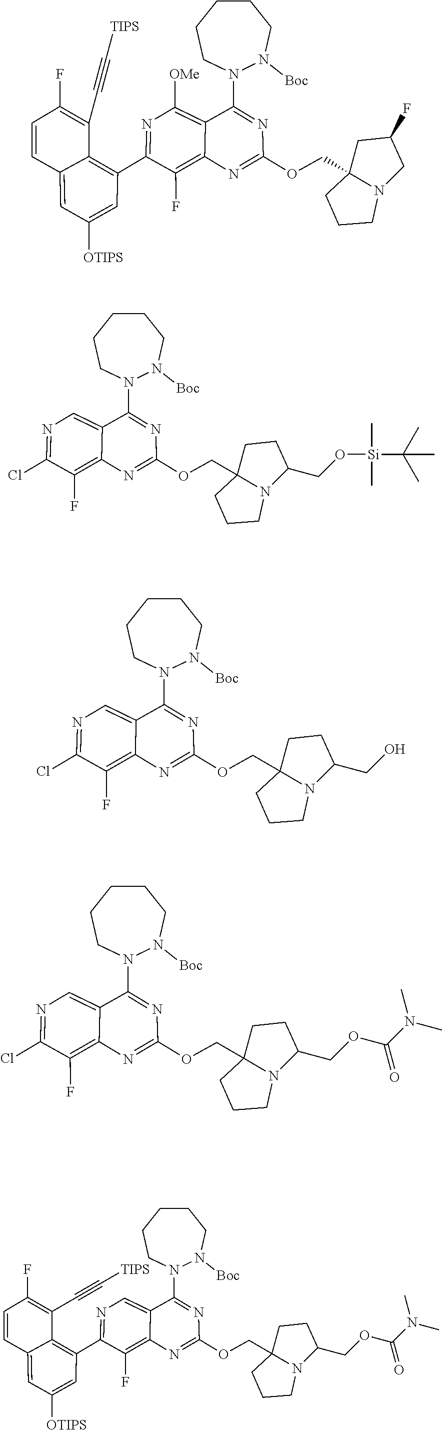

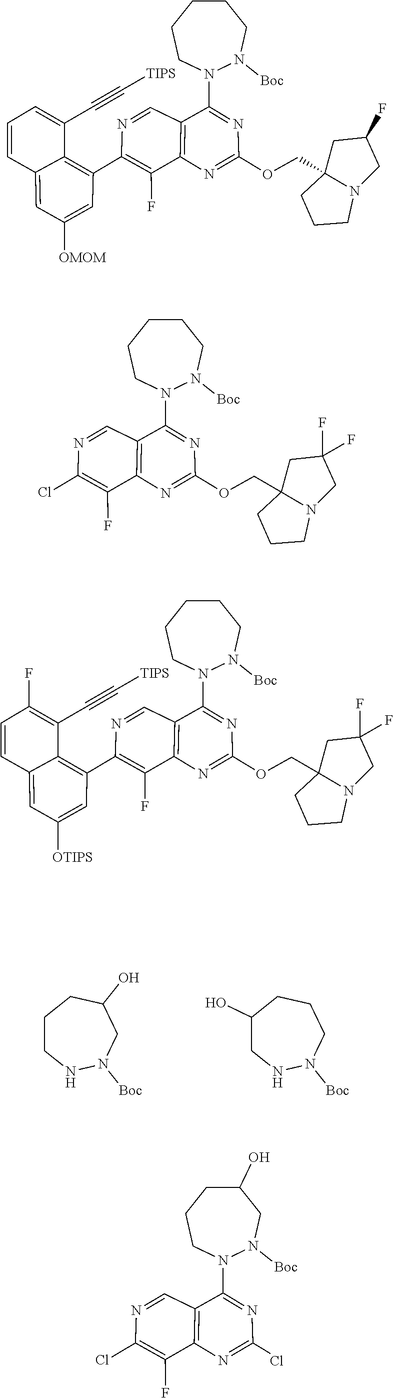

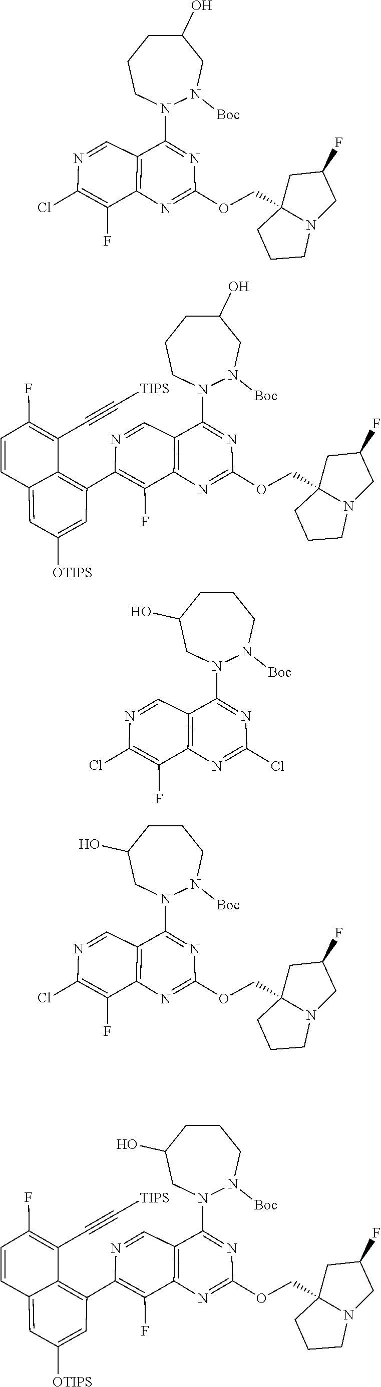

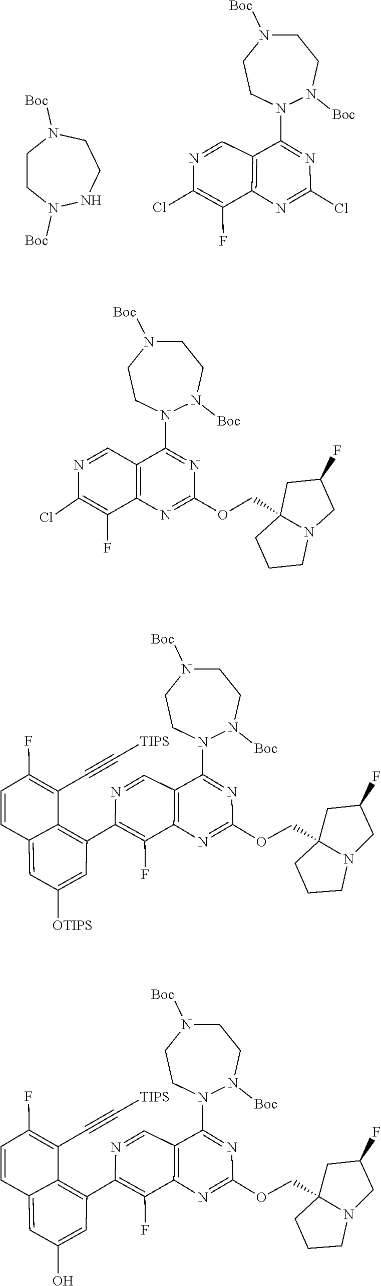

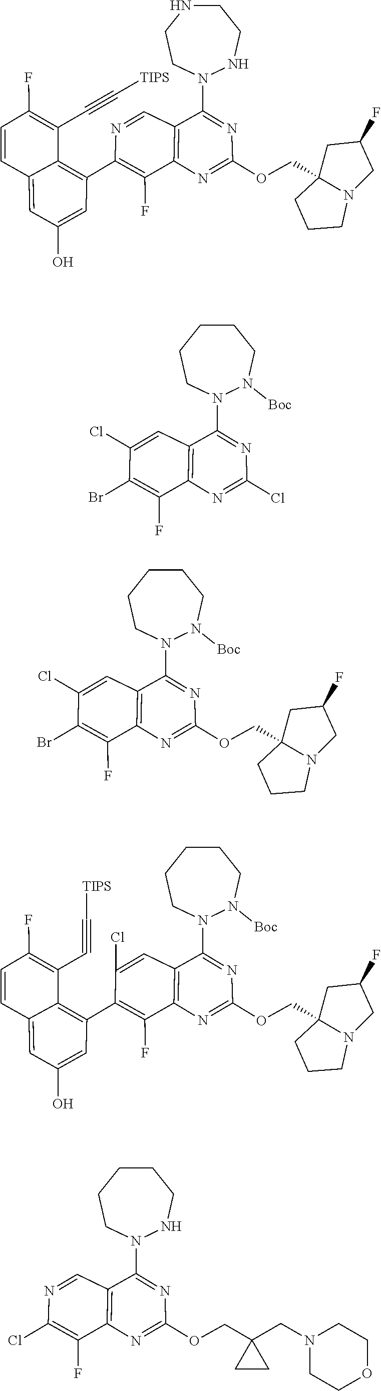

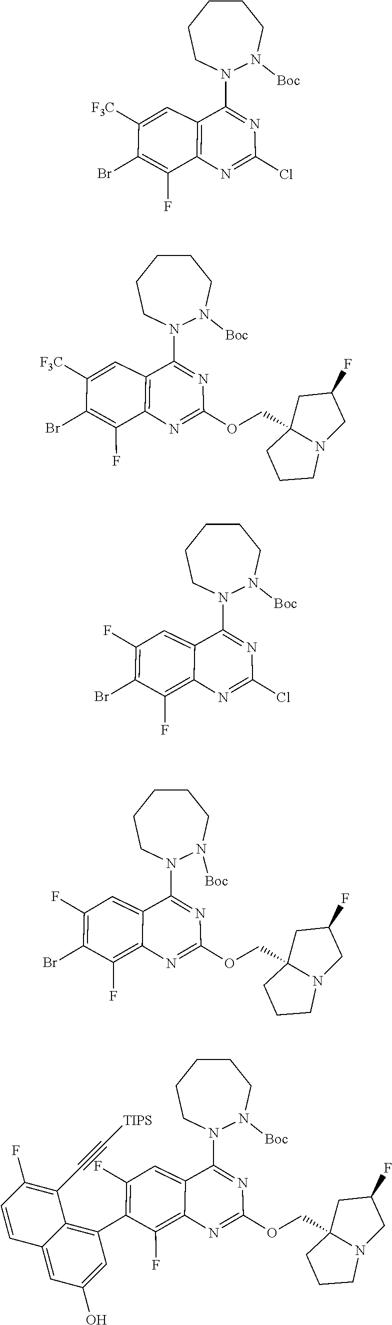

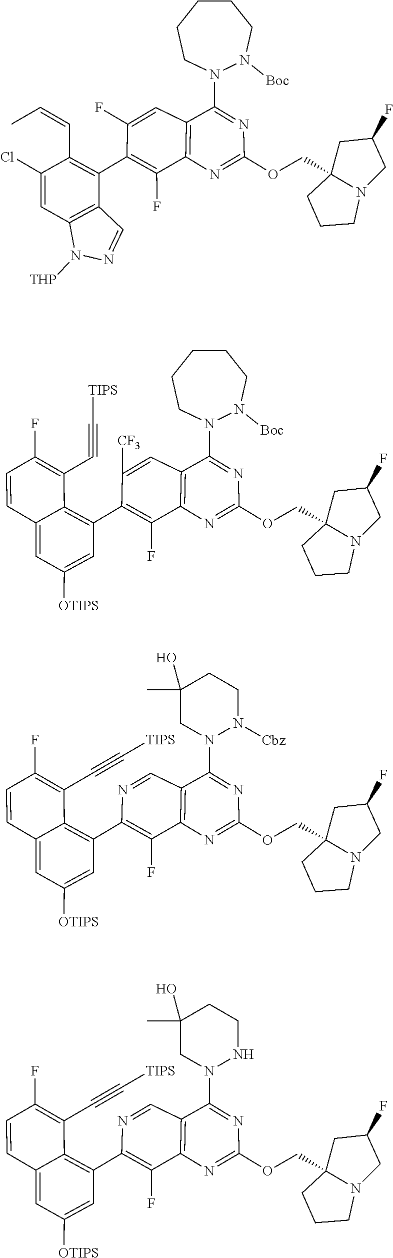

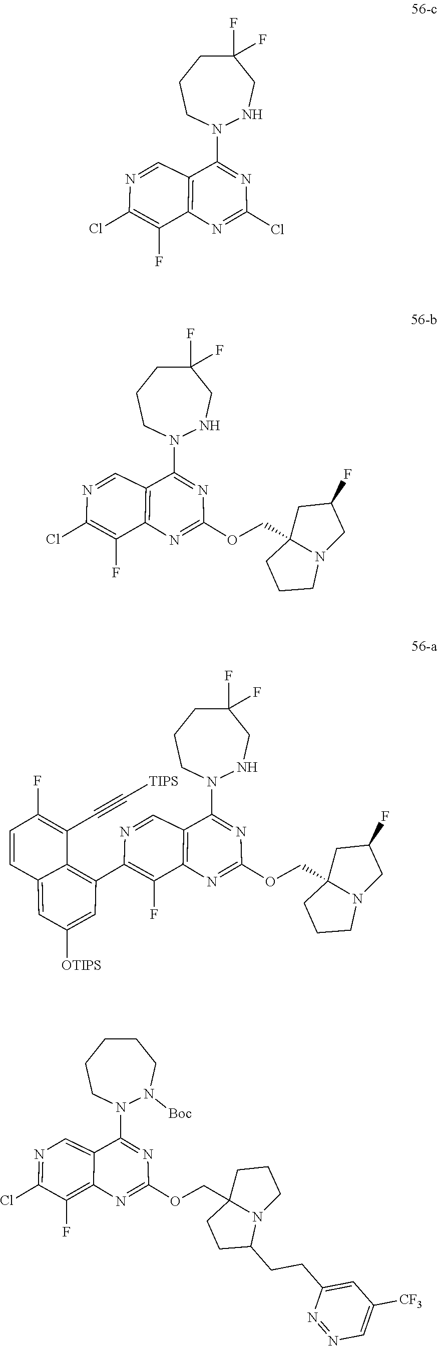

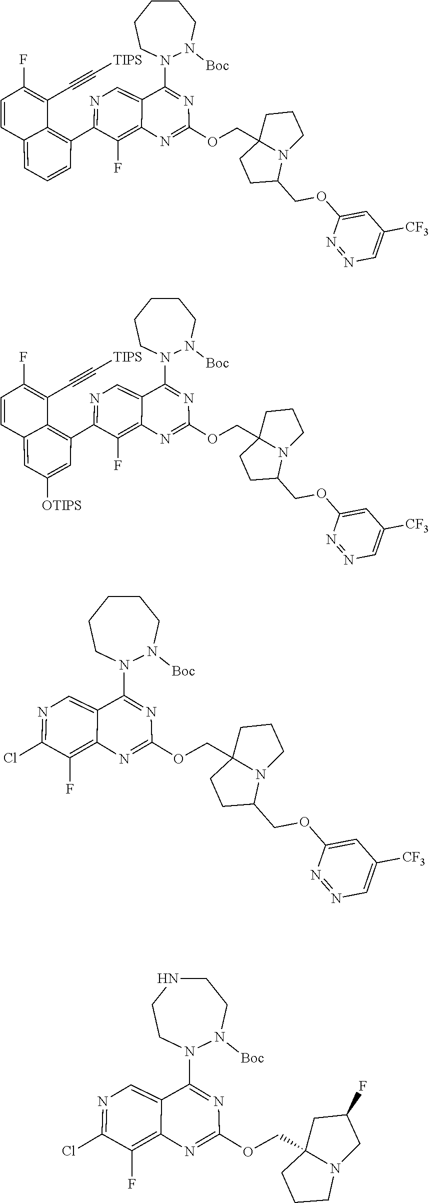

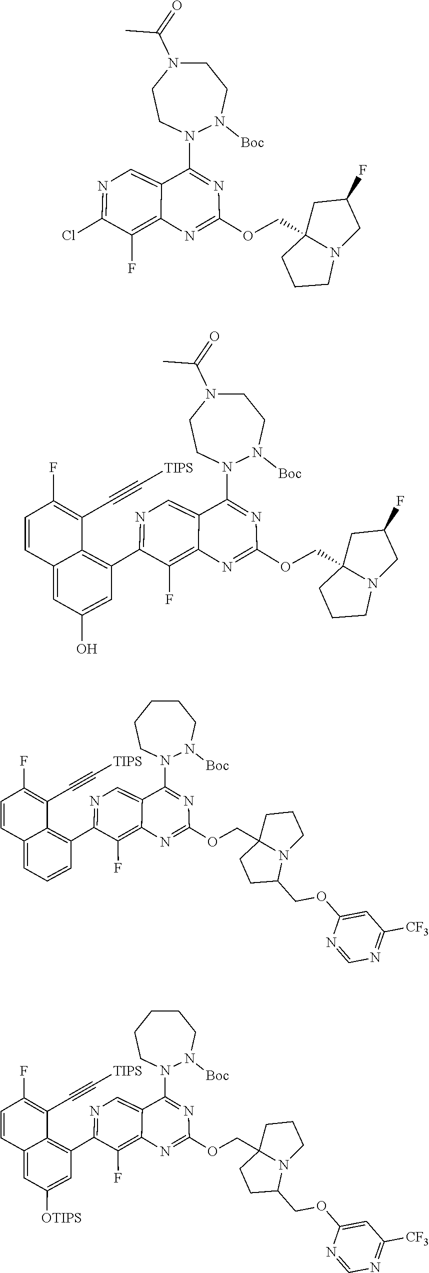

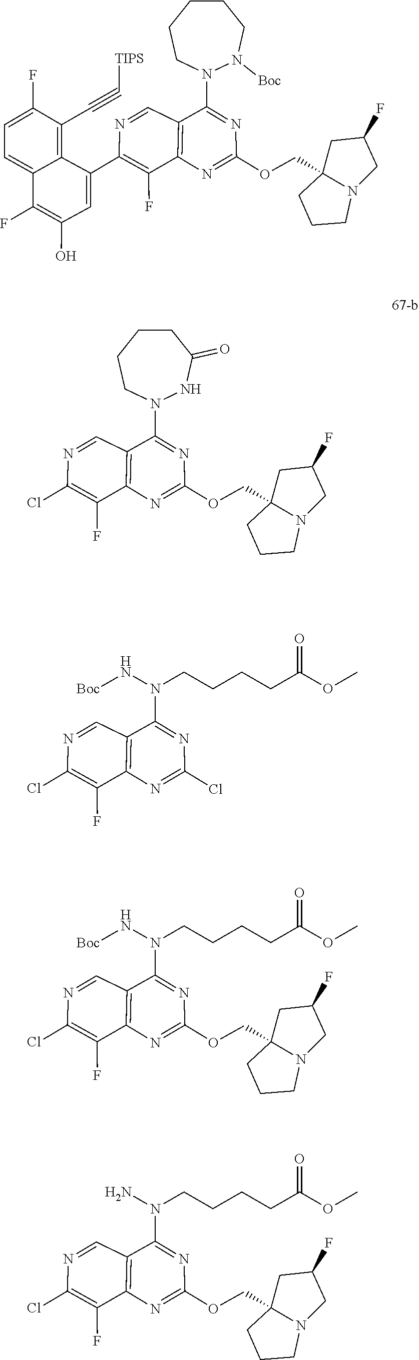

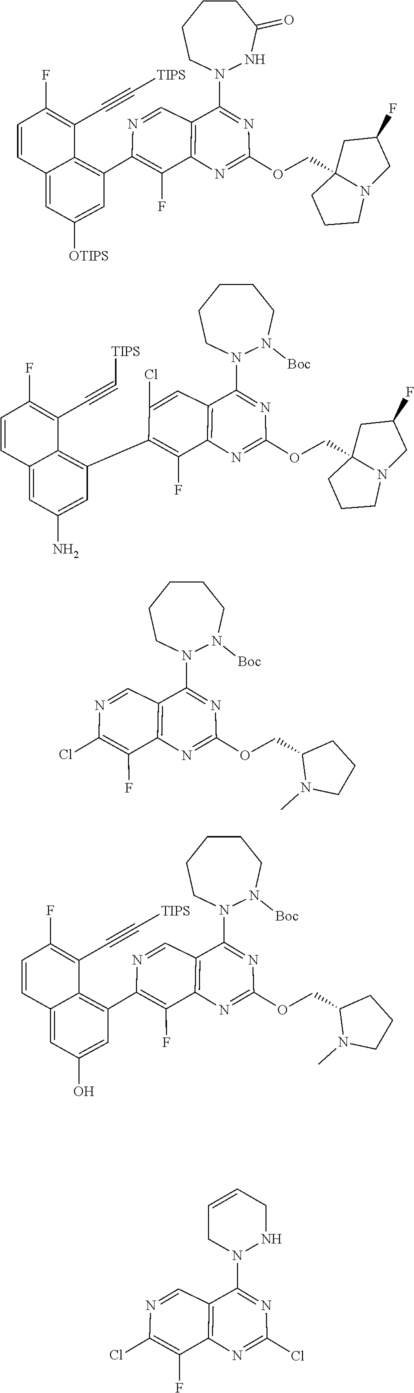

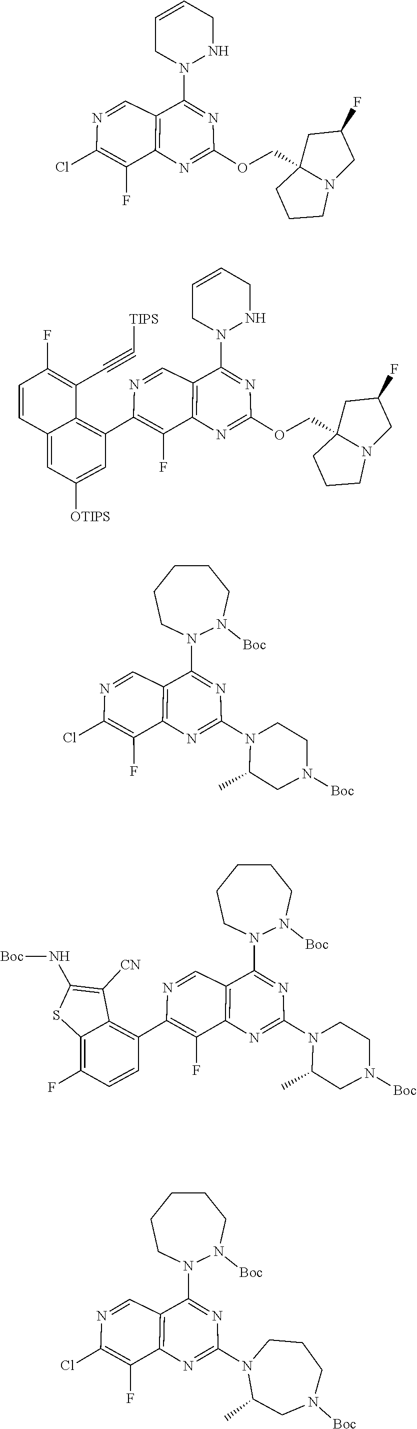

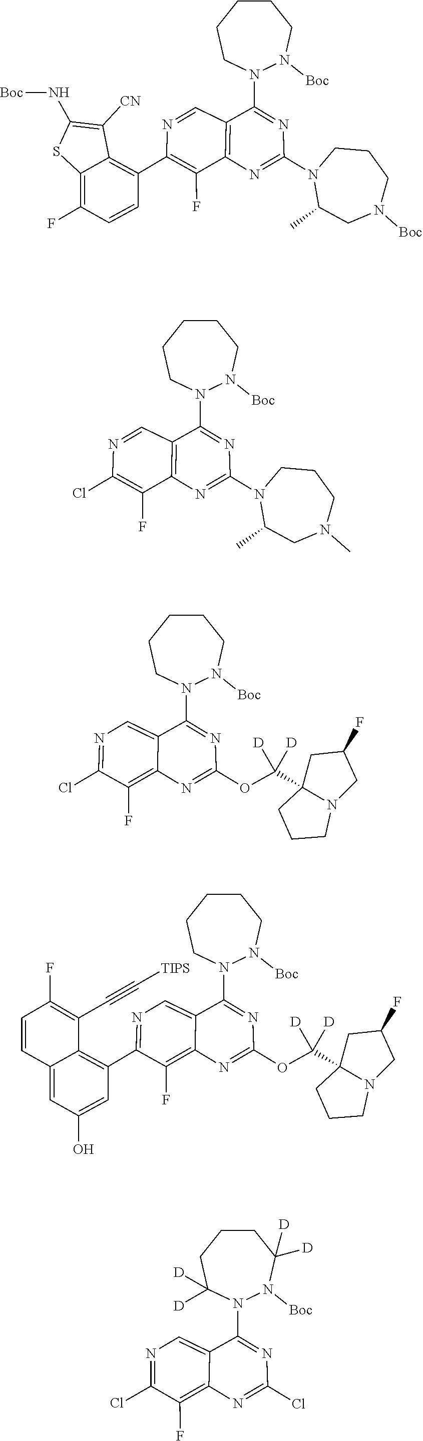

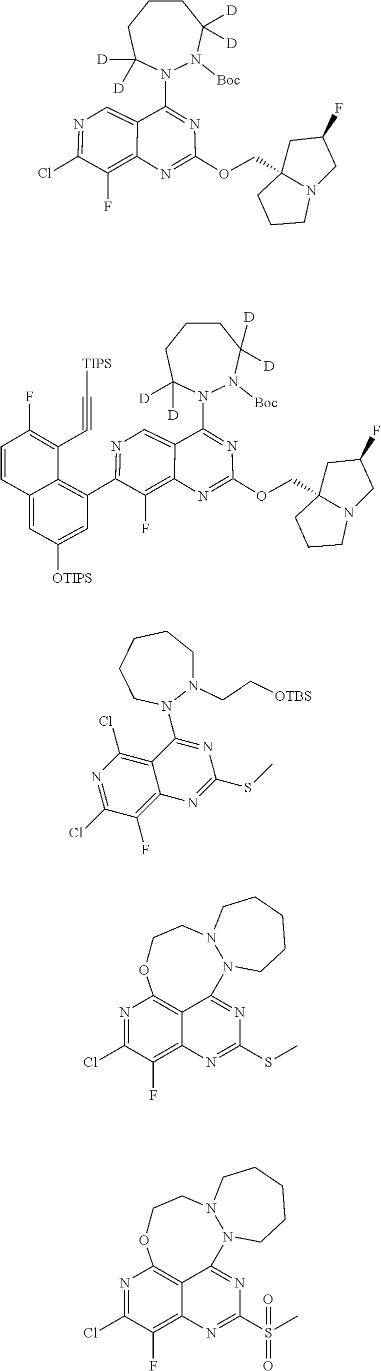

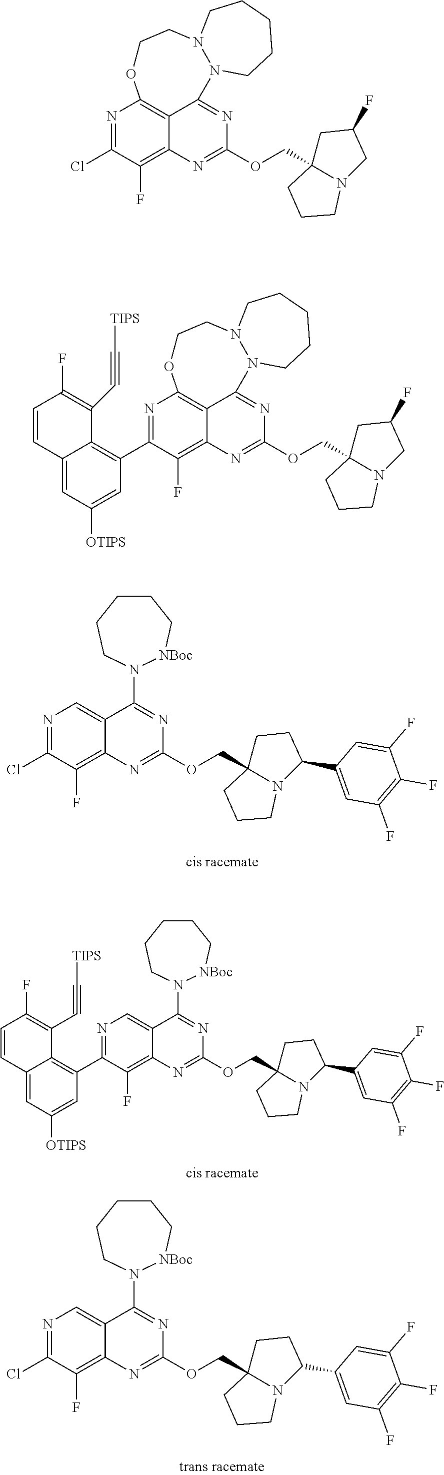

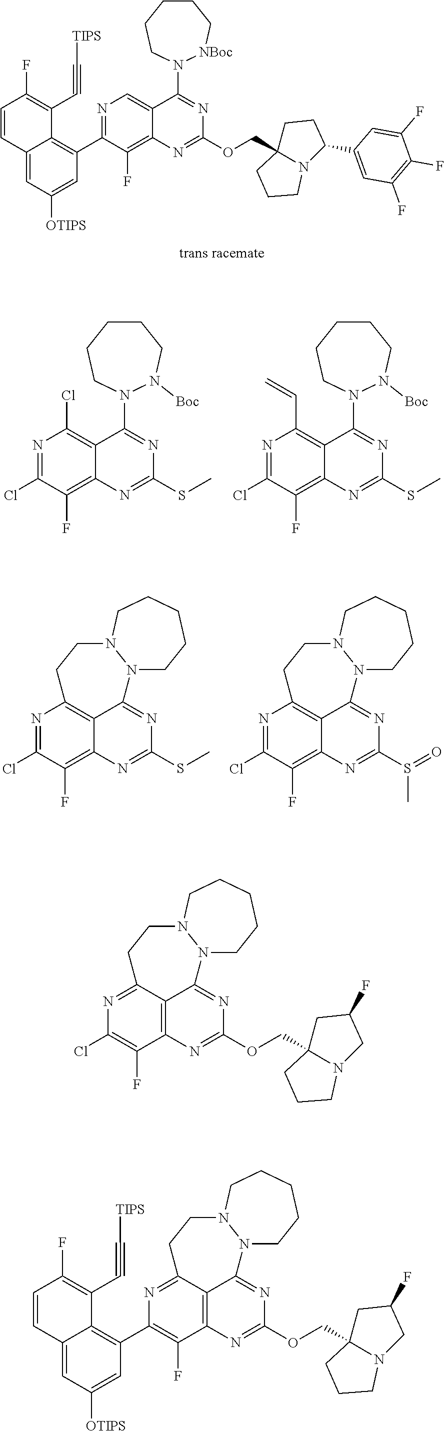

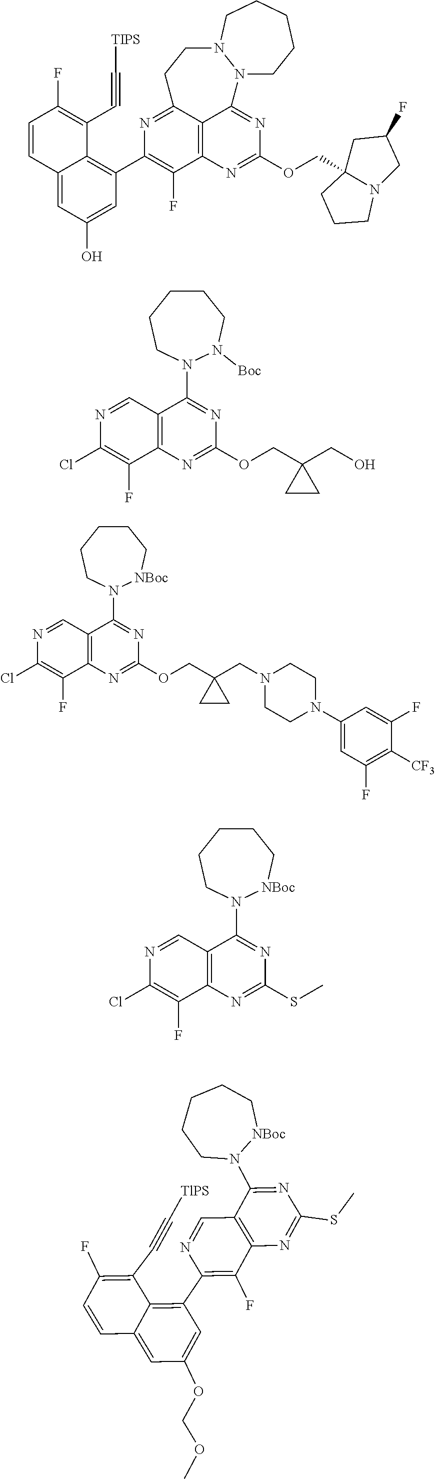

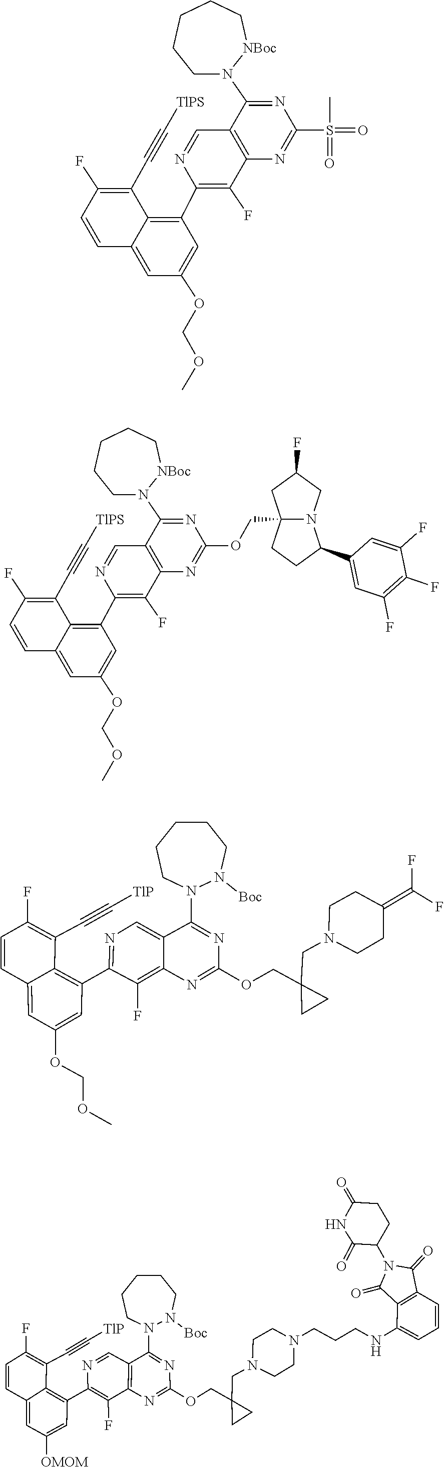

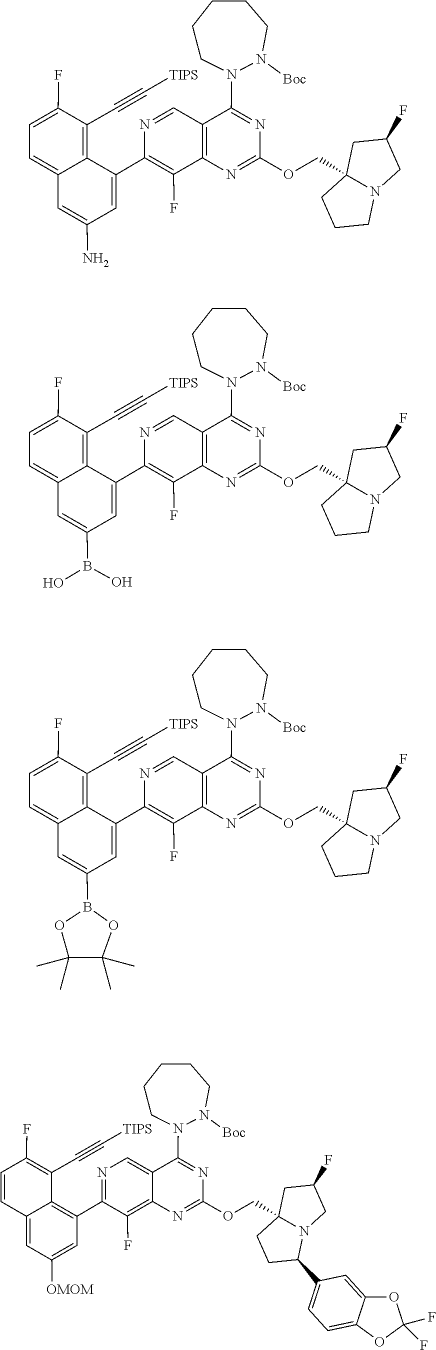

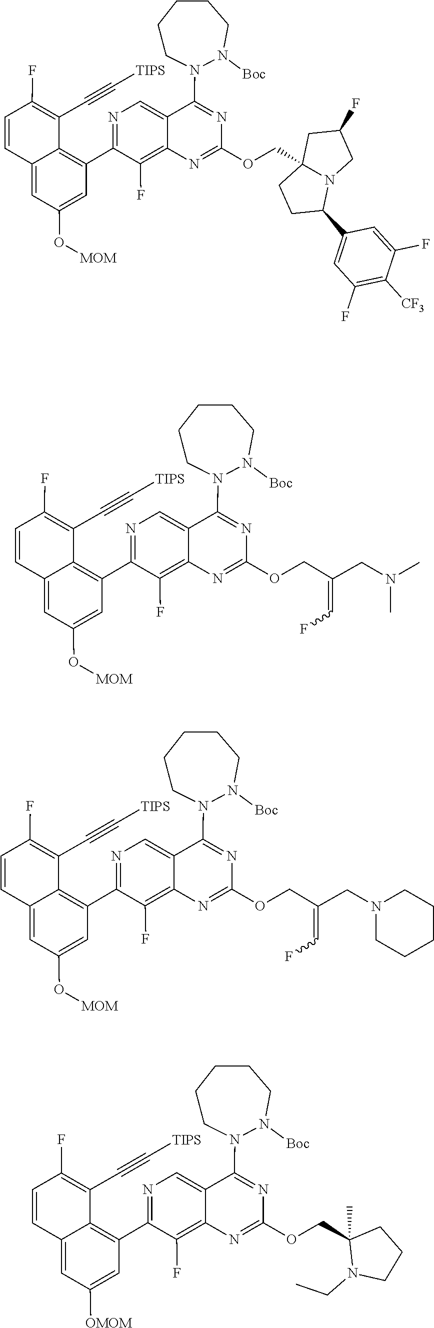

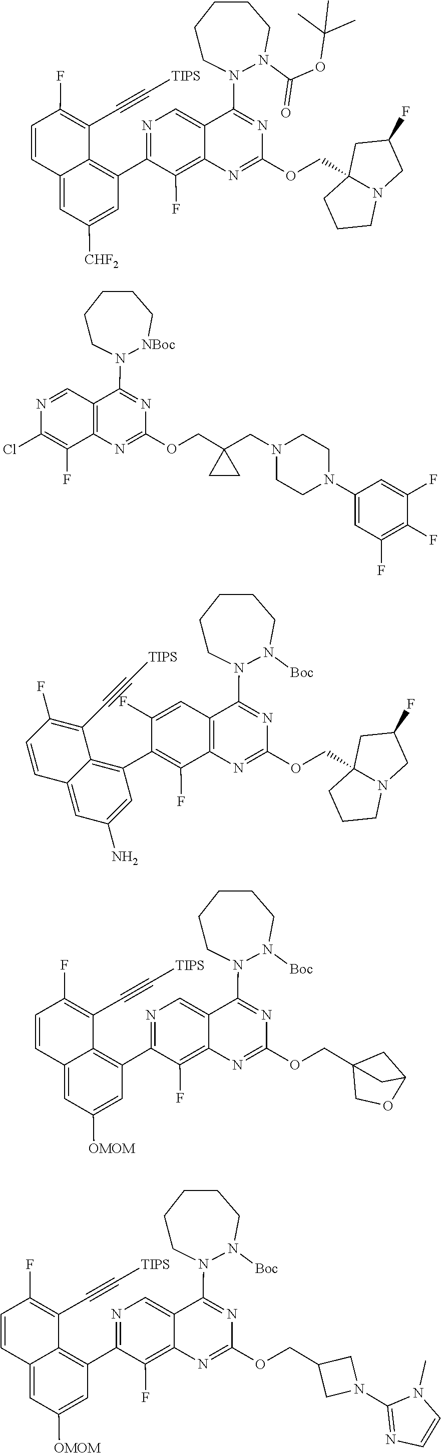

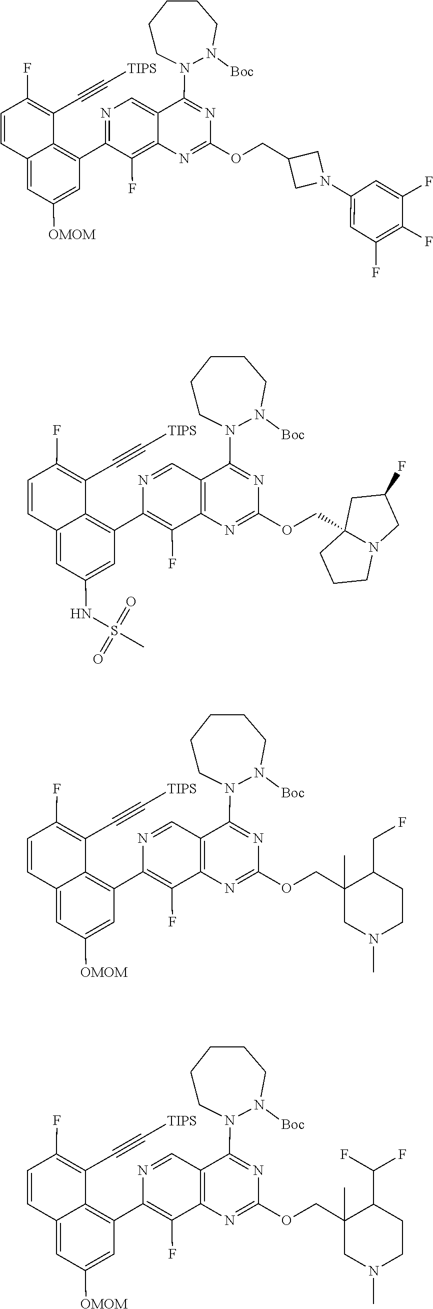

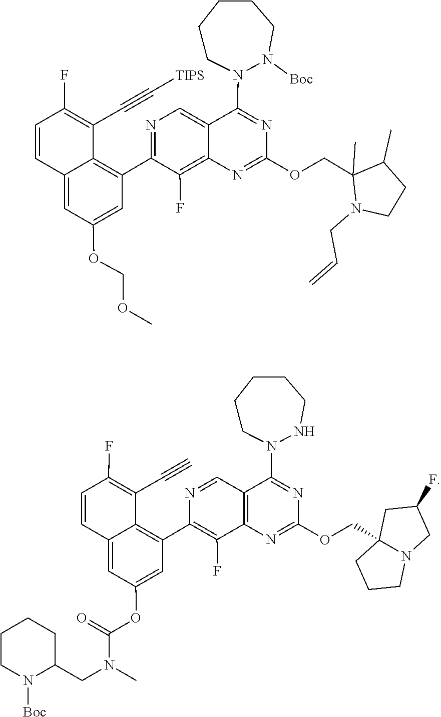

10. A pharmaceutical composition comprising a substance A and a pharmaceutically acceptable excipient, wherein the substance A is a therapeutically effective amount of the nitrogen-containing heterocyclic compound of formula I, the pharmaceutically acceptable salt thereof, the prodrug thereof, the stereoisomer thereof, the tautomer thereof or the isotopically labeled compound thereof according to claim 1.

11. A method for inhibiting RAS in a subject in need thereof, comprising: administering a therapeutically effective amount of a substance A to the subject, wherein the substance A is the compound containing structure of a nitrogen-containing heterocyclic compound of formula I, the pharmaceutically acceptable salt thereof, the prodrug thereof, the stereoisomer thereof, the tautomer thereof or the isotopically labeled compound thereof according to claim 1. The RAS is wild type RAS or mutated forms of RAS; the wild type RAS is wild-amplified KRAS; the mutated forms of RAS are a KRAS, HRAS or NRAS mutation.

12. A method for treating or preventing a RAS-related disease in a subject in need thereof, comprising: administering a therapeutically effective amount of a substance A to the subject, wherein the substance A is the compound containing structure of the nitrogen-containing heterocyclic compound of formula I, the pharmaceutically acceptable salt thereof, the prodrug thereof, the stereoisomer thereof, the tautomer thereof or the isotopically labeled compound thereof according to claim 1; the cancer is selected from the group consisting of colon cancer, appendiceal cancer, pancreatic cancer, MYH-related polyposis, hematologic cancer, breast cancer, endometrial cancer, gallbladder cancer, bile duct cancer, prostate cancer, lung cancer, brain cancer, ovarian cancer, cervical cancer, testicular cancer, kidney cancer, head or neck cancer, bone cancer, skin cancer, rectal cancer, liver cancer, esophageal cancer, stomach cancer, thyroid cancer, bladder cancer, lymphoma, leukemia and melanoma.

13. A method for treating or preventing cancer in a subject in need thereof, comprising: administering a therapeutically effective amount of a substance A to the subject, wherein the substance A is the compound containing structure of the nitrogen-containing heterocyclic compound of formula I, the pharmaceutically acceptable salt thereof, the prodrug thereof, the stereoisomer thereof, the tautomer thereof or the isotopically labeled compound thereof according to claim 1; the cancer is selected from the group consisting of colon cancer, appendiceal cancer, pancreatic cancer, MYH-related polyposis, hematologic cancer, breast cancer, endometrial cancer, gallbladder cancer, bile duct cancer, prostate cancer, lung cancer, brain cancer, ovarian cancer, cervical cancer, testicular cancer, kidney cancer, head or neck cancer, bone cancer, skin cancer, rectal cancer, liver cancer, esophageal cancer, stomach cancer, thyroid cancer, bladder cancer, lymphoma, leukemia and melanoma.
